(19) United States
(12) Patent Application Publication
(10) Pub. No.: US 2026/0199484 A1
(43) Pub. Date: Jul. 16, 2026

(54) PROTAC DEGRADERS OF MLLT1 AND/OR MLLT3

(71) Applicant: DARK BLUE THERAPEUTICS LTD, Oxford (GB)

(72) Inventors: Iain SIMPSON, Oxford (GB); Sarah MAJOR, Oxford (GB); Benjamin RAHEMTULLA, Oxford (GB); Ryan TINSON, Oxford (GB); Katherine JONES, Oxford (GB); Charlene FALLAN, Oxford (GB)

(73) Assignee: DARK BLUE THERAPEUTICS LTD, Thousand Oaks (GB)

(21) Appl. No.: 19/557,794

(22) Filed: Mar. 5, 2026

Related U.S. Application Data
(63) Continuation of application No. PCT/EP2025/067408, filed on Jun. 20, 2025.

(30) Foreign Application Priority Data
Jun. 21, 2024 (EP) 24183691.5
Jun. 21, 2024 (EP) 24183693.1

(51) Int. Cl. A61K 47/54; A61P 35/02

(52) U.S. Cl. CPC A61K 47/545; A61P 35/02

(57) ABSTRACT

The invention relates to a compound which is a Proteolysis Targeting Chimera (PROTAC) or a pharmaceutically acceptable salt thereof, wherein the PROTAC has the structure:

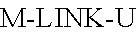

wherein U is an E3 ubiquitin ligase binding moiety, LINK is a moiety that covalently links M and U, and M is an MLLT1 and/or MLLT3 binder of formula (I):

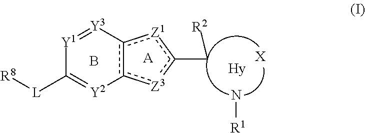

wherein:
wherein Z¹, Z², Y¹, Y², Y³, R¹, R², R⁸, X, L and Hy are as defined herein, and either R⁸ is a bond to LINK, or M is bonded to LINK via a C or N atom within group R⁸ or ring Hy such that a hydrogen atom on the C or N atom within group R⁸ or ring Hy is replaced with a bond to LINK.

The compounds are useful in the treatment of cancer.

### CROSS REFERENCE TO RELATED APPLICATIONS

This application is a continuation of International Application No. PCT/EP2025/067408, filed Jun. 20, 2025, which claims the benefit of priority to EP Patent Application No. 24183691.5, filed Jun. 21, 2024 and to EP Patent Application No. 24183693.1, filed Jun. 21, 2024.

### FIELD OF THE INVENTION

The present invention relates to compounds that find use in the treatment of cancer by inducing selective degradation of MLLT1 and/or MLLT3. The invention also provides such compounds per se, pharmaceutical compositions comprising such compounds, and methods of treating cancer by administering such compounds.

### BACKGROUND

The processes controlling gene transcription are highly regulated during development and normal homeostasis. Dysregulation of gene transcription is a common driver of cancer with somatic defects in proteins that control gene transcription a frequent occurrence (Cell, 2013, 153, 17; Cell, 2017, 168, 629). Transcriptional elongation is a central step in gene transcription, carried out by the RNA polymerase II (PolII), which itself is kept under tight control by regulatory protein complexes. During development PolII is recruited to sites of the genome proximal to the transcription start site for target genes and is kept in a paused state. Such target genes are often immediate response genes such as heat-shock genes and key developmental genes. Productive elongation is initiated by the coordinated interplay of regulatory protein complexes including the super elongation complex (SEC). The SEC includes multiple proteins with diverse functions including protein phosphorylation, histone reader, histone modification and regulatory activities. Critical to the recruitment of the SEC to target genes are the histone reader proteins MLLT1 (mixed lineage leukaemia translocated to 1, also known as eleven-nineteen leukaemia, ENL) and MLLT3 (mixed lineage leukaemia translocated to 3, also known as AF-9). MLLT1 and 3 contain essential YEATS domains that bind acetylated histones. Mutations in the YEATS domains reduce loading of Poll on to SEC target genes and suppression of gene transcription. (Cell Mol Life Sci 2018, 75, 3931; Nat Rev Mol Cell Biol 2012, 13, 543).

Gain-of-function mutations in the MLLT1 Yeats domain have been causally associated with Wilm's tumor (also known as nephroblastoma) a kidney cancer most commonly observed in children (Nature 2020, 577, 121). Additionally, it has been shown that for certain acute leukaemias MLLT1 represents a critical dependency (Nature 2017, 543, 270). Such leukaemias include mixed-lineage leukaemia (MLL) rearranged leukaemia. MLL rearrangements arise from in frame fusions of the MLL gene with more than 80 different partner genes, many of which are involved in the regulation of transcription elongation including components of the SEC (Front. Pediatr. 5:4. doi: 10.3389/fped.2017.00004). MLL rearrangements are observed in approximately 10% of all acute leukaemias including a high frequency in infantile ALL where it accounts for 70-80% of all cases (Front. Pediatr. 5:4. doi: 10.3389/fped.2017.00004). Notably it is reported that patients with MLL rearranged leukaemias have an especially poor prognosis (New Engl J Med 2016, 374, 2209). Target genes for MLL-SEC complexes include potent oncogenes such as BCL-2, Myc and CDK6 along with many other genes implicated in maintaining cancer cell self-renewal, growth and survival, such as the HOX family genes and MEIS1 (Front. Pediatr. 5:4. doi: 10.3389/fped.2017.00004). Consistent with a role in the regulation of multiple oncogenes, it has been demonstrated that the SEC can play a critical role in the transcriptional addiction of both hematopoietic and solid cancers. For example, data supports potential in non-MLL rearranged leukaemia (Cancer Disc 2022, 12, 2684) and it is reported that with some breast cancer cells, growth and survival is dependent on a SEC mediated transcription of the Myc oncogene (Cell Rep. 2021 Feb. 16; 34(7):108749. doi: 10.1016/j.celrep.2021.108749. PMID: 33596420; PMCID: PMC8006859).

Taken together the evidence supports MLLT1 and/or MLLT3 as an attractive therapeutic target across acute leukaemias and solid cancers.

Proteolysis targeting chimeras (PROTACs) have been proposed as a small molecule-based platform technology capable of inducing proteolysis of a target protein in the body. The PROTAC is a bifunctional compound in which a molecule that binds to a disease-related target protein and an E3 ubiquitin ligase binding moiety are linked by a chemical linker. Theoretically, the PROTAC compound is capable of inducing degradation of the target protein by placing the disease-related target protein near the E3 ubiquitin ligase.

Drug discovery efforts have resulted in agents that either bind the YEATS domain to block the MLLT1 and/or MLLT3 protein to histone interaction or that result in degradation of the MLLT1 and/or MLLT3 protein (using a PROTAC approach). In all cases however, the reported activity has been weak (ACS Cent Sci 2021, 7, 815; Cancer Disc 2022, 12, 2684; Angew. Chem. Int. Ed. 2018, 57, 16302). Accordingly, there remains a high need to identify potent modulators of MLLT cell function.

### SUMMARY OF THE INVENTION

The inventors have discovered a series of compounds that induce selective degradation of MLLT1 and/or MLLT3. Accordingly, the invention provides a compound which is a Proteolysis Targeting Chimera (PROTAC) or a pharmaceutically acceptable salt thereof, wherein the PROTAC has the structure:

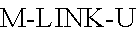

wherein U is an E3 ubiquitin ligase binding moiety, LINK is a moiety that covalently links M and U, and M is an MLLT1 and/or MLLT3 binder of formula (I):

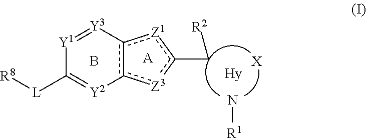

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
each R¹¹ is independently selected from H, C₁₋₄ alkyl, and C₁₋₄ cycloalkyl;
wherein M is bonded to LINK via a C or N atom within group R⁸ such that a hydrogen atom on the C or N atom within group R⁸ is replaced with a bond to LINK.

In a preferred embodiment, M is of formula (II):

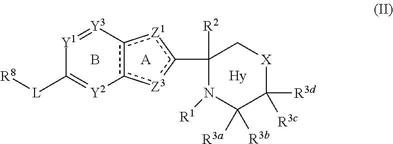

wherein:
Z¹, Z³, Y¹, Y², Y³, R¹, R², R⁸, X and L are as defined herein;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)-then neither R^{3c} nor R^{3d} are halo;
wherein at least two of R^{3a}, R^{3b}, R^{3c} and R^{3d} are H; and
wherein M is bonded to LINK via a C or N atom within group R⁸ such that a hydrogen atom on the C or N atom within group R⁸ is replaced with a bond to LINK.

In a more preferred embodiment, M is of formula (III):

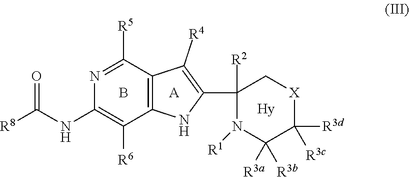

wherein:
R¹ is H or C₁₋₄ alkyl which is itself unsubstituted or substituted with one C₁₋₄ alkoxy or one, two or three halo, preferably with one C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3a} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring,
with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo, C₁₋₄ alkoxy, and C₁₋₄ alkyl which is itself unsubstituted or substituted by one, two or three halo;
R⁵ and R⁶ are independently selected from H, halo, C₁₋₄ alkoxy, and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
wherein M is bonded to LINK via a C or N atom within group R⁸ such that a hydrogen atom on the C or N atom within group R⁸ is replaced with a bond to LINK.

In a particularly preferred embodiment, M is of formula (IV):

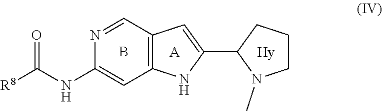

wherein R⁸ is an 8- to 10-membered heteroaryl ring, the group R⁸ being unsubstituted or substituted by one, two or three substituents independently selected from halo, C₁₋₄ alkyl and R¹⁰;
R¹⁰ is a 3-membered cycloalkyl ring;
wherein M is bonded to LINK via a C or N atom within group R⁸ such that a hydrogen atom on the C or N atom within group R⁸ is replaced with a bond to LINK. As discussed elsewhere herein, the stereochemistry of the compounds of the invention at the ring Hy is preferably such that the bond from ring Hy to the core ring A is in the “up” position and the bond from ring Hy to R² is the down position. Thus, formula (IV) preferably has the stereochemistry depicted below:

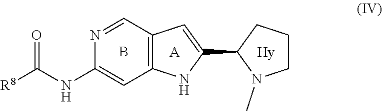

The inventors have also surprisingly found that compounds of formula (I′) are potent inhibitors of MLLT1 and MLLT3. Accordingly, the invention provides a compound which is a bicyclic compound of formula (I′) or a pharmaceutically acceptable salt thereof:

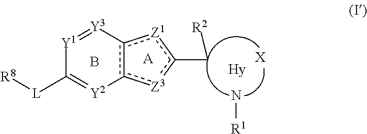

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
at least one of R⁴, R⁵ and R⁶ is not H;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
each R¹¹ is independently selected from H, C₁₋₄ alkyl, and C₁₋₄ cycloalkyl.

In a preferred embodiment, the compound is a bicyclic compound of formula (II′) or a pharmaceutically acceptable salt thereof:

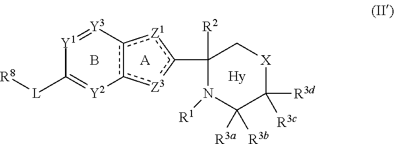

wherein:
Z¹, Z³, Y¹, Y², Y³, R¹, R², R⁸, X and L are as defined in any one of claims 1 to 18;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, with the proviso that when X is —N(R¹¹)—, O, S, —S(O)₂— or —S(O)(NR¹¹)—, then neither R^{3c} nor R^{3d} are halo;
or
R^{3a} and R^{3c} are H, and R^{3b} and R^{3d} form, together with the C atoms to which they are attached, a C₅₋₆ cycloalkyl ring;
wherein at least two of R^{3a}, R^{3b}, R^{3c} and R^{3d} are H.

In a more preferred embodiment, the compound is a bicyclic compound of formula (III′) or a pharmaceutically acceptable salt thereof:

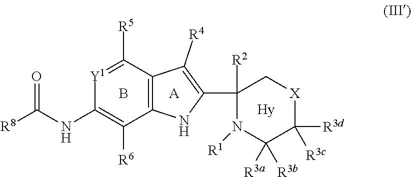

wherein:
X, Y¹, R¹, R², R⁴, R⁵, R⁶, R^{3a}, R^{3b}, R^{3c}, R^{3a} and R⁸ are as defined herein.

The present invention also provides a compound as described herein for use in a method of treating cancer in a subject in need thereof. Also provided is a method for treating cancer in a subject, which method comprises administering to said subject an effective amount of a compound as described herein. Further provided is the use of a compound as described herein in the manufacture of a medicament for use in treating cancer in a subject.

### BRIEF DESCRIPTION OF THE DRAWINGS

FIG. 1: Graph showing reduction in tumour growth in in vivo mouse model of human AML, following oral administration of the compound of Example 1, following the procedure described in Example 112.

### DETAILED DESCRIPTION OF THE INVENTION

### Definitions

As used herein, a C₁₋₄ alkyl group is a linear or branched alkyl group containing from 1 to 4 carbon atoms. A C₁₋₄ alkyl group is often a C₁₋₃ alkyl group. Examples of C₁₋₄ alkyl groups include methyl, ethyl, n-propyl, iso-propyl, n-butyl, sec-butyl, and tert-butyl. A C₁₋₃ alkyl group is typically a C₁₋₂ alkyl group. A C₁₋₂ alkyl group is methyl or ethyl, typically methyl. For the avoidance of doubt, where multiple alkyl groups are present, the alkyl groups may be the same or different.

As used herein, a C₁₋₄ alkoxy group is typically a said C₁₋₄ alkyl group which is joined to the rest of the molecule via an oxygen atom. Typically, a C₁₋₄ alkoxy group is a C₁₋₃ alkoxy group. Examples of C₁₋₄ alkoxy groups include methoxy, ethoxy, propoxy and butoxy. Typically, a C₁₋₃ alkoxy group is a C₁₋₂ alkoxy group such as a methoxy or ethoxy group. For the avoidance of doubt, where two alkoxy groups are present, the alkoxy groups may be the same or different.

As used herein, a C₁₋₃₀ alkylene group is a linear or branched divalent alkyl group that contains from 1 to 30 carbon atoms. A C₁₋₃₀ alkylene group is sometimes a C₁₋₂₀ alkylene group, and often a C₁₋₁₀ alkylene group. C₁₋₃₀ alkylene groups, C₁₋₂₀ alkylene groups and C₁₋₁₀ alkylene groups are preferably linear.

A C₁₋₃₀ alkylene group is sometimes a C₁₋₆ alkylene group, typically a C₁₋₄ alkylene group or a C₁₋₃ alkylene group. Examples of C₁₋₄ alkylene groups include methylene, ethylene, n-propylene, iso-propylene, n-butylene, sec-butylene, and tert-butylene. A C₁₋₃ alkylene group is typically a C₁₋₂ alkylene group. A C₁₋₂ alkylene group is methylene or ethylene, typically methylene. For the avoidance of doubt, where multiple alkylene groups are present, the alkylene groups may be the same or different.

As used herein, a C₂₋₆ alkenylene group is a linear or branched divalent alkenyl group containing from 2 to 6 carbon atoms and having one or more, e.g. one or two, typically one double bonds. Typically, a C₂₋₆ alkenylene group is a C₂₋₄ alkenylene group. Examples of C₂₋₄ alkenylene groups include divalent ethenylene, propenylene and butenylene. For the avoidance of doubt, where multiple alkenylene groups are present, the alkenylene groups may be the same or different.

An alkyl, alkoxy, alkylene or alkenylene group as used herein may be unsubstituted or substituted. Unless otherwise stated, substituted alkyl, alkoxy, alkylene or alkenylene groups typically carry one or more, e.g. one, two or three e.g. one, or two, e.g. one substituent selected from halo, OH, and unsubstituted C₁₋₄ alkoxy. Preferred substituents are halo and C₁₋₄ alkoxy unless otherwise stated. The substituents on a substituted alkyl, alkoxy, alkylene or alkenylene group are typically themselves unsubstituted. Where more than one substituent is present, these may be the same or different.

As used herein, a halo typically refers to chlorine, fluorine, bromine or iodine, preferably chlorine, bromine or fluorine, more preferably chorine or fluorine, most preferably fluorine unless otherwise stated.

A C₃₋₈ cycloalkyl ring is a cyclic hydrocarbon containing from 3 to 8 carbon atoms. A cycloalkyl ring may be saturated or partially unsaturated, but is typically saturated. A C₃₋₈ cycloalkyl ring is typically a C₃₋₆ cycloalkyl ring. A partially unsaturated cycloalkyl ring is a cyclic hydrocarbon containing 1 or 2, e.g. 1 double bond. C₃₋₆ cycloalkyl and C₅₋₆ cycloalkyl rings may also be referred to herein as 3- to 6-membered cycloalkyl rings and 5- to 6-membered cycloalkyl rings respectively.

A C₃₋₆ cycloalkyl ring may be a saturated C₃₋₆ cycloalkyl ring. A C₃₋₆ cycloalkyl ring may be a C₅₋₆ cycloalkyl ring, in particular a saturated C₅₋₆ cycloalkyl ring. Examples of C₃₋₆ cycloalkyl rings are cyclopropyl, cyclobutyl cyclopentyl and cyclohexyl groups.

A C₃₋₈ cycloalkyl ring may be a C₇₋₈ cycloalkyl ring, in particular a saturated C₇₋₈ cycloalkyl ring. Examples of C₇₋₈ cycloalkyl rings are cycloheptanyl, cyclooctanyl, bicyclo[2.2.1]heptanyl and bicyclo[2.2.2]octanyl groups.

A 5- to 10-membered heterocyclyl ring is a cyclic group containing from 5 to 10 atoms selected from C, O, N and S in the ring, including at least one heteroatom, and typically one or two heteroatoms unless otherwise stated. The heteroatom or heteroatoms are typically selected from O, N, and S, most typically from S and N, especially N. For example, where the heterocyclyl ring is denoted a nitrogen-containing heterocyclyl group, it contains one nitrogen atom and optionally a further heteroatom selected from O, N and S. A heterocyclyl ring may be saturated or partially unsaturated, but is typically saturated. A 5- to 10-membered partially unsaturated heterocyclyl ring is a cyclic group containing from 5 to 10 atoms selected from C, O, N and S in the ring and containing 1 or 2, e.g. 1 double bond. Typically, in the compounds described herein, a heterocyclyl ring is saturated unless otherwise specified.

A 5- to 10-membered heterocyclyl ring is typically a 4- to 7-membered heterocyclyl ring or a 5- to 6-membered ring. A 4- to 7-membered heterocyclyl ring is typically a monocyclic ring and may be a monocyclic 5- or 6-membered heterocyclyl ring. Alternatively, a 5- to 10-membered heterocyclyl ring may be a 9- or 10-membered fused bicyclic heterocyclyl ring (i.e. a fused heterobicyclic group). In some compounds described herein, a 5- to 10-membered heterocyclyl group is a 4- to 7-membered nitrogen-containing heterocyclyl ring which is unsubstituted or is substituted as described herein. Preferred 4- to 7-membered nitrogen-containing heterocyclyl rings include morpholine, pyrrolidine, piperidine and piperazine.

Examples of 5- and 6-membered saturated heterocyclyl rings include piperazine, piperidine, morpholine, diazinane and pyrrolidine. Diazinane is typically 1,4-diazinane.

Examples of 9- and 10-membered bicyclic heterocyclyl rings include azaspiro[4.4]nonane and octahydro-1H-indole. Preferably, the bicyclic heterocyclyl ring comprises 1, 2 or 3, preferably 1 or 2 nitrogen atoms.

For the avoidance of doubt, references to a heterocyclyl ring also include fused polycyclic ring systems, including for instance fused bicyclic systems in which a heterocyclyl ring is fused to an aryl group. When the heterocyclyl ring is such a fused heterocyclyl group, preferred examples are fused ring systems wherein a 5- to 6-membered heterocyclyl group is fused to a phenyl group.

As used herein, a 6- to 10-membered aryl ring is a substituted or unsubstituted, monocyclic or fused polycyclic aromatic group containing from 6 to 10 carbon atoms in the ring portion. Examples include monocyclic groups such as phenyl and fused bicyclic groups such as naphthyl and indenyl. Phenyl (benzene) is preferred.

As used herein, a 5- to 10-membered heteroaryl ring is a substituted or unsubstituted monocyclic or fused polycyclic aromatic group containing from 5 to 10 atoms in the ring portion, including at least one heteroatom, for example 1, 2 or 3 heteroatoms, typically selected from O, S and N. A heteroaryl ring is typically a 5- or 6-membered heteroaryl ring or an 8- to 10-membered heteroaryl ring. Preferably, the heteroaryl ring comprises 1, 2 or 3, preferably 1 or 2 nitrogen atoms.

Examples of 5- and 6-membered heteroaryl rings include thiazole, pyrazole, pyrimidine, triazole, 1,2,4-oxadiazole and pyrazine.

Examples of 8-, 9- and 10-membered heteroaryl rings include indazole, thieno[2,3-c]pyrazole, imidazo[4,5-b]pyridine, pyrazolo[3,4-b]pyridine, furo[2,3-c]pyridine, indole, benzoxazole, benzothiazole, [1,2,4]triazolo[4,3-a]pyridine, thieno[2,3-d]pyrimidine, 1,2,3-benzotriazole, imidazo[1,5-a]pyridine, imidazo[1,2-a]pyrazine, oxazolo[5,4-b]pyridine, quinoline, naphthyridine, isoquinoline, quinazoline, and quinoxaline. 8-, 9- and 10-membered heteroaryl rings as used herein are typically fused bicyclic groups.

For the avoidance of doubt, references to a heteroaryl ring also include fused polycyclic ring systems, including for instance fused bicyclic systems in which a heteroaryl ring is fused to an aryl group. When the heteroaryl ring is such a fused heteroaryl group, preferred examples are fused ring systems wherein a 5- to 6-membered heteroaryl group is fused to a phenyl group. Indazole is preferred.

As used herein, a fused bicyclic group is a group comprising two cyclic moieties sharing a common bond between two atoms.

A cycloalkyl, heterocyclyl, aryl or heteroaryl ring may be unsubstituted or substituted as described herein unless otherwise stated. For example, a cycloalkyl, heterocyclyl, aryl or heteroaryl ring may be unsubstituted or substituted with 1, 2 or 3, typically 1 or 2 such as e.g. 1 substituent. Suitable substituents include halo, C₁₋₄alkoxy, R¹⁰, —(C₁₋₄ alkylene)-R¹⁰, ═O, —CN, and C₁₋₄ alkyl, wherein R¹⁰ is as defined herein). The substituents on a substituted cycloalkyl, heterocyclyl, aryl or heteroaryl ring are typically themselves unsubstituted, unless otherwise stated.

The compounds described herein comprise at least one heterocyclyl ring comprising at least one nitrogen atom. Said nitrogen atom(s) are independently selected from secondary, tertiary and quaternary nitrogen atom(s). A quaternary nitrogen atom is present when the compound comprises a quaternised derivative of one or more monocyclic groups or fused bicyclic groups. As used herein, a quaternised derivative of a moiety such as a cyclic moiety is formed by bonding an additional alkyl group to a nitrogen atom in the moiety such that the valency of the said nitrogen atom increases from 3 to 4 and the nitrogen atom is positively charged.

The compounds described herein comprise a heterocyclyl ring identified as ring Hy, which is a 4- to 7-membered heterocyclyl ring. Ring Hy contains X and at least one N atom in the ring portion, wherein X is a bond, —N(R¹¹)—, O, S, —S(O)₂—, —S(O)(NR¹¹)—, or —C(R¹¹)₂—, and R¹¹ is as defined herein. Ring Hy is linked to ring A (as identified in formula (I) above) via a C atom within ring Hy, said C atom also being linked to R² which is as defined herein. A N atom within ring Hy is substituted by R¹ which is as defined herein.

The rest of ring Hy is unsubstituted or substituted by one or two R³. Each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring, as defined herein. In option (ii), the skilled person would understand that the C atom to which the two R³ are attached is a spiro atom (i.e. the common atom that connects the two rings of a spiro compound).

As used herein, the terms “monovalent” or “monovalent moiety” are used to describe a chemical group obtainable by removing a hydrogen atom from the corresponding compound. As used herein, the terms “divalent” or “divalent moiety” are used to describe a chemical group obtainable by removing a hydrogen atom from the corresponding monovalent moiety. Thus, as used herein, the terms “divalent” and “divalent moiety” are used to describe a chemical group obtainable by removing two hydrogen atoms from the corresponding compound.

It is to be understood that each individual atom present in the formulae depicted herein may be present in the form of any of its naturally occurring isotopes, with the most abundant isotope(s) being preferred. Thus, by way of example, each individual hydrogen atom present in the formulae depicted herein may be present as a 1H, 2H (deuterium) or 3H (tritium) atom, preferably 1H. Similarly, by way of example, each individual carbon atom present in the formulae depicted herein may be present as a 12C, 13C or 14C atom, preferably 12C.

In the compounds of the invention, the stereochemistry is not limited. In particular, where moiety M of formula (I) and/or moiety U contains one or more chiral centre, the compounds may be used in enantiomerically or diasteriomerically pure form, or in the form of a mixture of isomers. Further, for the avoidance of doubt, the compounds of the invention may be used in any tautomeric form. Typically, the agent or substance described herein contains at least 50%, preferably at least 60, 75%, 90% or 95% of a compound which is enantiomerically or diasteriomerically pure. Thus, the compound is preferably substantially optically pure.

The moiety M of formula (I) typically contains at least one chiral centre at the carbon atom of ring Hy which is linked to R² and to ring A. Moiety M may be provided in the form of the R-enantiomer at said carbon atom, in the form of the S-enantiomer at said carbon atom, or in the form of a mixture of the two enantiomers. The substance or agent described herein may contain at least 50%, preferably at least 60, 75%, 90% or 95% of a compound which is enantiomerically or diasteriomerically pure at moiety M. A pure enantiomeric form of either the R- or the S-enantiomer may be preferred. In some embodiments, the R-enantiomer at said carbon atom is preferred, and in particular when R² is H then the R-enantiomer at said carbon atom is preferred. The preferred stereochemistry at said carbon atom is also depicted by the following illustrative structure of formula (I) where the bond from ring Hy to the core ring A is in the “up” position and the bond from ring Hy to R² is the down position:

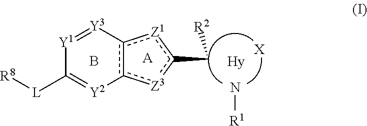

This stereochemistry at the carbon atom of ring Hy which is linked to R² and to ring A, where the bond from ring Hy to the core ring A is in the “up” position and the bond from ring Hy to R² is the down position (as depicted above), is also preferred for the moiety M of formulae (II), (III), (IV), and for the compounds of formulae (I′), (II′) and (III′).

As discussed further herein, where the compounds of the invention contain a chiral centre, and in particular where moiety M and/or moiety U contains a chiral centre, the individual stereoisomers may be obtained by chiral synthesis, or by separation of stereoisomers. Stereoisomers may be separated, for example, using chiral chromatography. The individual stereoisomers may be identified using the IUPAC labels R and S as appropriate. Alternatively, the individual stereoisomers may be identified by the order in which they elute, for example the first, second, third or fourth stereoisomer respectively, as obtained by separation using chiral chromatography. Where two or more stereocentres are present in a compound or moiety, a combination of IUPAC nomenclature for stereocentres where absolute stereochemistry has been defined, and rate of elution, may be used.

For example, where two or more individual stereoisomers are unidentified by IUPAC nomenclature, these stereoisomers may be identified as the first- and second-eluting isomers, as obtained by separation using chiral chromatography. Therefore, the stereoisomers at a chiral carbon atom of ring Hy which is linked to R² and to ring A may be defined as the first-eluting and second-eluting stereoisomers, as obtained by separation using chiral chromatography. Chiral chromatography is typically reverse phase HPLC or SFC.

The compounds of the present invention may exist in unsolvated as well as solvated forms with pharmaceutically acceptable solvents such as water, ethanol, and the like, and it is intended that the invention embrace both solvated and unsolvated forms.

A compound of the present invention can be converted into a pharmaceutically acceptable salt thereof, and a salt can be converted into the free compound, by conventional methods. For instance, a compound of the present invention can be contacted with a pharmaceutically acceptable acid to form a pharmaceutically acceptable salt. A pharmaceutically acceptable salt is a salt with a pharmaceutically acceptable acid or base.

Pharmaceutically acceptable acids include both inorganic acids such as hydrochloric, sulphuric, phosphoric, diphosphoric, hydrobromic or nitric acid and organic acids such as oxalic, citric, fumaric, maleic, malic, ascorbic, succinic, tartaric, benzoic, acetic, methanesulphonic, ethanesulphonic, benzenesulphonic orp-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines. Hydrochloride salts and acetate salts are preferred, in particular hydrochloride salts.

### Proteolysis Targeting Chimera (PROTAC)s

The compounds of the invention comprise a binder of MLLT1 and/or MLLT3 (M) that is covalently attached via a linker moiety (LINK) to an E3 ubiquitin ligase binding moiety (U). The inventors have surprisingly discovered that such compounds may have substantially enhanced efficacy compared with a corresponding MLLT1 and/or MLLT3 inhibitor that is not attached to an E3 ubiquitin ligase binding moiety. For example, the antiproliferative effects of the compounds may be substantially enhanced by covalent attachment to the E3 ubiquitin ligase binding moiety. Such compounds may induce selective degradation of MLLT1 and/or MLLT3. For instance, such compounds may induce selective degradation of MLLT1 and/or MLLT3 without inducing degradation of other proteins, such as FLT3 (fms like tyrosine kinase 3). Such compounds may also not inhibit FLT3.

Thus, the invention provides a compound which is a Proteolysis Targeting Chimera (PROTAC) or a pharmaceutically acceptable salt thereof, wherein the PROTAC has the structure:

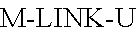

wherein U is an E3 ubiquitin ligase binding moiety, LINK is a moiety that covalently links M and U, and M is an MLLT1 and/or MLLT3 binder of formula (I) as defined herein.

In the compounds of the present invention, the MLLT1 and/or MLLT3 binder is covalently attached to the E3 ubiquitin ligase binding moiety via the moiety labelled LINK. LINK is either a single bond or a chemical group that covalently attaches the MLLT1 and/or MLLT3 binder to the E3 ubiquitin ligase binding moiety.

The MLLT1 and/or MLLT3 binder is attached to LINK via a C or N atom within group R⁸ or ring Hy (as described herein) such that a hydrogen atom on the C or N atom within group R⁸ or ring Hy is replaced with a bond to LINK. A hydrogen atom on the C or N atom within group R⁸ or ring Hy is replaced by either a direct single bond to the E3 ubiquitin ligase binding moiety or by a chemical group that covalently attaches the MLLT1 and/or MLLT3 binder to the E3 ubiquitin ligase binding moiety. It is preferred for the MLLT1 and/or MLLT3 binder to be attached to LINK via a C or N atom within group R⁸.

The description herein discusses the substitution of group R⁸ and ring Hy by various chemical groups aside from LINK. For completeness, the skilled person would readily appreciate that where group R⁸ and/or ring Hy are defined as being unsubstituted, this is intended as a reference to the absence of substituents other than a bond to LINK. Therefore, such a definition retains the option that group R⁸ or ring Hy is attached to LINK via a C or N atom within group R⁸ or ring Hy such that a hydrogen atom on the C or N atom within group R⁸ or ring Hy is replaced with a bond to LINK. Furthermore, the skilled person would readily appreciate that where group R⁸ and/or ring Hy are defined as being substituted by one or more chemical groups (suitable such chemical groups are defined herein), this is intended as a reference to substituents other than a bond to LINK. Therefore, such a definition includes group R⁸ or ring Hy being attached to LINK via a C or N atom within group R⁸ or ring Hy such that a hydrogen atom on the C or N atom within group R⁸ or ring Hy is replaced with a bond to LINK. Therefore, where group R⁸ and/or ring Hy are defined as being unsubstituted or substituted by one or more chemical groups (suitable such chemical groups are defined herein), then one of group R⁸ or ring Hy may also be attached to LINK via a C or N atom within group R⁸ or ring Hy such that a hydrogen atom on the C or N atom within group R⁸ or ring Hy is replaced with a bond to LINK. A bond to LINK may optionally be positioned on a carbon atom which carries a further substituent (where chemically possible). Typically, a carbon or nitrogen atom which is bonded to LINK does not carry a further substituent.

For the avoidance of doubt, where group R⁸ and/or ring Hy are defined as being substituted by a hydrogen atom (e.g. where R¹, R², R¹¹, R^{3a}, R^{3b}, R^{3c} and R^{3d} are defined as being a hydrogen atom), then a reference herein to a hydrogen atom within group R⁸ or ring Hy being replaced with a bond to LINK, includes the option that said hydrogen atom (e.g. a hydrogen atom at R¹, R², R¹¹, R^{3a}, R^{3b}, R^{3c} and R^{3d}) is replaced with a bond to LINK. Therefore, where R¹, R², R¹¹, R^{3a}, R^{3b}, R^{3c} and R^{3d} are defined as being a hydrogen atom, then said hydrogen atom may be replaced with a bond to LINK.

For the avoidance of doubt, where ring Hy or group R⁸ is substituted, a C or N atom which is bonded to LINK may be within the aryl, heteroaryl, heterocyclyl, or cycloalkyl ring of ring Hy or group R⁸, or it may be a C or N atom of a substituent on ring Hy or group R⁸. Preferably, a C or N atom which is bonded to LINK is a C or N atom on the heterocyclyl ring of ring Hy (i.e. directly to the ring and not via a substituent), a C or N atom of the heteroaryl ring of group R⁸, or the heteroaryl, cycloalkyl, heterocyclyl or phenyl ring of R¹⁰ in the case that a group R¹⁰ is present as, or as part of, a substituent on R⁸.

As the skilled person would readily appreciate, methods for preparation of compounds (in which two discrete chemical entities contribute discrete biological functions to the overall compound) are very well known in the art. A hugely diverse range of techniques for covalently attaching the respective chemical entities, via a vast number of chemical linker moieties, is well established. Such is the ubiquity of these methodologies, that standard text books devoted entirely to this topic have long been available. One such textbook is “Bioconjugate Techniques” (Greg T. Hermanson, Academic Press Inc., 1996), the content of which is herein incorporated by reference in its entirety.

MLLT1 and/or MLLT3 Binder Group M

In the compounds of the invention, the MLLT1 and/or MLLT3 binder M is a monovalent moiety of formula (I) as defined herein.

In the description that follows, the skilled person will readily understand that M is a monovalent moiety that is bonded to LINK via a C or N atom within group R⁸ or ring Hy (as described herein) such that a hydrogen atom on the C or N atom within group R⁸ or ring Hy is replaced with a bond to LINK. Thus, in the description that follows, any of the hydrogen atoms on any of the C or N atoms within group R⁸ or ring Hy may be removed to form the point of attachment of the monovalent moiety M to LINK.

Typically, ring Hy is a 5- to 6-membered heterocyclyl ring containing X and at least one N atom, for instance one or two N atoms. X is typically a bond, —N(R¹¹)—, O or —C(R¹)₂—. Preferably, X is a bond, —N(Me)-, O or —CH₂—, most preferably X is a bond. In particular, when ring Hy is a 4- or 5-membered heterocyclyl ring, X is most preferably a bond. For example, ring Hy may be selected from a pyrrolidinyl, piperidinyl, morpholinyl or diazinanyl ring. Preferably, ring Hy is a 5-membered heterocyclyl ring containing X and at least one N atom, for instance one N atom. For example, ring Hy may be a pyrrolidinyl ring.

Ring Hy is linked to ring A via a C atom within ring Hy, said C atom also being linked to R². A N atom within ring Hy is substituted by R¹. Typically, the N atom of ring Hy which is substituted by R¹ is adjacent to the C atom of ring Hy that is bonded to ring A.

A C or N atom within ring Hy may be bonded to LINK by a hydrogen atom on that C or N atom being replaced by a bond to LINK. Typically, where ring Hy is bonded to LINK, this is via a C atom on Hy. Preferably, the bond to LINK is via replacement of a hydrogen atom on a C or N within group R⁸, i.e. the bond to LINK is preferably not via ring Hy.

Typically, R¹ is H, C₁₋₄ cycloalkyl, or C₁₋₄ alkyl which is itself unsubstituted or substituted with one C₁₋₄ alkoxy or one, two or three halo, preferably with one C₁₋₄ alkoxy, for example R¹ may be H or C₁₋₂ alkyl which is unsubstituted or substituted with one C₁₋₂ alkoxy or one, two or three halo, preferably with one C₁₋₂ alkoxy, or R¹ may be H or C₁₋₂ alkyl which is unsubstituted or substituted with one C₁₋₂ alkoxy. Preferably, R¹ is H, methyl, ethyl or methoxyethyl, more preferably methyl or ethyl. Most preferably, R¹ is methyl.

R² is H or methyl. Preferably, R² is H.

Typically, each R¹¹ is independently selected from H and methyl. When X is —N(R¹¹)— or —S(O)(NR¹¹)—, R¹¹ is preferably methyl. When X is —C(R¹¹)₂—, optionally one R¹¹ is replaced with a single bond to LINK and the other R¹¹ is H. When X is —C(R¹¹)₂—, each R¹¹ is preferably H.

Aside from groups R¹ and R², ring Hy may further be unsubstituted or substituted by one or two R³. Typically, each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring. More typically, each R³ is independently selected from methyl, ethyl, t-butyl methoxy and fluoro, or (i) two R³ linked to adjacent C atoms in ring Hy form, together with the C atoms to which they are attached, a cyclohexyl ring, or (ii) two R³ linked to the same C atom in ring Hy form, together with the C atom to which they are attached, a cyclopentyl ring. Preferably, ring Hy is substituted by no R³ groups.

In another typical embodiment, each R³ is independently selected from methyl, ethyl, t-butyl, methoxy and fluoro, or (i) two R³ linked to adjacent C atoms in ring Hy form, together with the C atoms to which they are attached, a cyclohexyl ring, or (ii) two R³ linked to the same C atom in ring Hy form, together with the C atom to which they are attached, a cyclopropyl ring or a cyclopentyl ring. Preferably, ring Hy is substituted by two R³ groups which are both C₁₋₄ alkyl, for instance both being methyl, or one R³ group which is C₁₋₄ alkyl, for instance methyl, ethyl or t-butyl.

Typically, therefore, ring Hy is a 5- to 6-membered heterocyclyl ring containing X and at least one N atom, for example a pyrrolidinyl, piperidinyl, morpholinyl or diazinanyl ring; the N atom of ring Hy which is substituted by R¹ is adjacent to the C atom of ring Hy that is bonded to ring A; R¹ is H or C₁₋₂ alkyl which is unsubstituted or substituted with one C₁₋₂ alkoxy or one, two or three halo, preferably with one C₁₋₂ alkoxy; R² is H or methyl; X is a bond, —N(R¹¹)—, O or —C(R¹¹)₂—; R¹¹ is H or methyl; ring Hy is further unsubstituted or substituted by one or two R³; and each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring.

Preferably, ring Hy is a pyrrolidinyl ring; the N atom of ring Hy which is substituted by R¹ is adjacent to the C atom of ring Hy that is bonded to ring A; R¹ is methyl or ethyl, more preferably methyl; R² is H; and ring Hy is substituted by no R³ groups, i.e. ring Hy is not further substituted and carries substituent R¹ only. In this embodiment, ring Hy may be referred to as being an unsubstituted pyrrolidine ring, wherein the skilled person would understand that ring Hy still carries substituent R¹. Alternatively, ring Hy is a pyrrolidinyl ring; the N atom of ring Hy which is substituted by R¹ is adjacent to the C atom of ring Hy that is bonded to ring A; R¹ is H, methyl, ethyl or methoxyethyl, more preferably methyl; R² is H; and ring Hy is further substituted by two R³ groups which are both C₁₋₄ alkyl, for instance both being methyl, or one R³ group which is C₁₋₄ alkyl, for instance methyl, ethyl or t-butyl.

Typically, no more than two of Y¹, Y² and Y³ are N. Preferably Y¹ is N, Y² is —C(R⁶)—, and Y³ is —C(R⁵)—.

Typically, Z¹ is —C(R⁴)—, Z³ is —N(H)—, and Y¹ is N. Thus, the bicyclic structure formed by rings A and B has the structure:

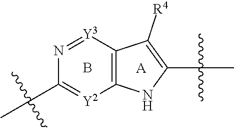

In one preferred embodiment, Z¹ is —C(R⁴)—, Z³ is —N(H)—, Y¹ is N, Y² is —C(R⁶)—, and Y³ is —C(R⁵)—, such that the bicyclic structure formed by rings A and B has the structure:

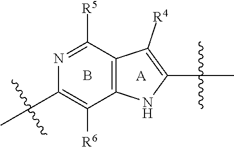

Typically, R⁴, R⁵ and R⁶ are independently selected from H, CN, halo, C₁₋₄ alkoxy, and C₁₋₄ alkyl which is itself unsubstituted or substituted by one, two or three halo, with the proviso that R⁵ and R⁶ are not CN. Typically, R⁴, R⁵ and R⁶ are independently selected from H, CN, halo (e.g. fluoro), methoxy, and methyl, with the proviso that R⁵ and R⁶ are not CN. Preferably, either R⁴, R⁵ and R⁶ are H, or one of R⁴, R⁵ and R⁶ is selected from halo, CN, C₁₋₄ alkoxy, and C₁₋₄ alkyl which is itself unsubstituted or substituted by one, two or three halo, and the rest are H, with the proviso that R¹ and R⁶ are not CN. More preferably, either R⁴, R⁵ and R⁶ are H, or one of R⁴, R¹ and R⁶ is selected from halo (e.g. fluoro), CN, methoxy, and methyl, and the rest are H, with the proviso that R¹ and R⁶ are not CN. More preferably, R⁴, R¹ and R⁶ are H.

In one preferred embodiment, R¹ and R⁶ are as defined herein and R⁴ is selected from H, halo, CN, C₁₋₄ alkoxy, and C₁₋₄ alkyl which is itself unsubstituted or substituted by one, two or three halo. More preferably, R⁵ and R⁶ are as defined herein and R⁴ is selected from H, halo, CN, C₁₋₄ alkyl and CF₃. Most preferably, R⁵ and R⁶ are as defined herein and R⁴ is selected from H, F, Cl, CN, Me and CF₃.

In one preferred embodiment, R⁵ is selected from H, cyclopropyl, C₁₋₄ alkoxy, and C₁₋₄ alkyl, for example from H, C₁₋₄ alkoxy, and C₁₋₄ alkyl. More preferably, R⁵ is selected from H, cyclopropyl, methoxy and methyl, for example H, methoxy and methyl.

In one preferred embodiment, R⁶ is H.

Typically, therefore, in formula (I):
R⁴ is selected from H, halo, CN, C₁₋₄ alkoxy, and C₁₋₄ alkyl which is itself unsubstituted or substituted by one, two or three halo, and R⁵ and R⁶ when present are H; or
R⁵ is selected from H, cyclopropyl, C₁₋₄ alkoxy, and C₁₋₄ alkyl, typically from H, C₁₋₄ alkoxy, and C₁₋₄ alkyl, and R⁴ and R⁶ when present are H; or
R⁴, R⁵ and R⁶ are H.

Preferably, in formula (I):
R⁴ is selected from H, F, Cl, Me, CN and CF₃, and R⁵ and R⁶ when present are H;
R⁵ is selected from H, cyclopropyl, methoxy and methyl, typically from H, methoxy and methyl, and R⁴ and R⁶ when present are H; or
R⁴, R⁵ and R⁶ are H.

In one alternative preferred embodiment, R⁵ and R⁶ are as defined herein and R⁴ is selected from halo, CN, C₁₋₄ alkoxy, and C₁₋₄ alkyl which is itself unsubstituted or substituted by one, two or three halo. More preferably, R⁵ and R⁶ are as defined herein and R⁴ is selected from halo, CN, C₁₋₄ alkyl and CF₃. More preferably, R⁵ and R⁶ are as defined herein and R⁴ is selected from halo, and CF₃. Most preferably, R⁵ and R⁶ are as defined herein and R⁴ is selected from F, Cl, and CF₃.

L is —C(O)N(H)—, wherein the C atom of L is bonded to R⁸, and the N atom of L is bonded to ring B.

R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹. R⁹ and R¹⁰ are as defined herein.

Typically, R⁸ is selected from indazolyl, thieno[2,3-c]pyrazolyl, imidazo[4,5-b]pyridinyl, pyrazolo[3,4-b]pyridinyl, furo[2,3-c]pyridinyl, indolyl, benzoxazolyl, benzothiazolyl, [1,2,4]triazolo[4,3-a]pyridinyl, thieno[2,3-d]pyrimidinyl, 1,2,3-benzotriazolyl, imidazo[1,5-a]pyridinyl, imidazo[1,2-a]pyrazinyl, oxazolo[5,4-b]pyridinyl, quinolinyl, naphthyridinyl, isoquinolinyl, quinazolinyl, and quinoxalinyl. Preferably, R⁸ is a 9- to 10-membered heteroaryl ring containing one, two or three N atoms. More preferably, R⁸ is an indazolyl group.

In some preferred embodiments, R⁸ is a group selected from the following structures. For the avoidance of doubt, the following structures may be unsubstituted or substituted by one, two or three substituents as defined herein.

### [See Below]

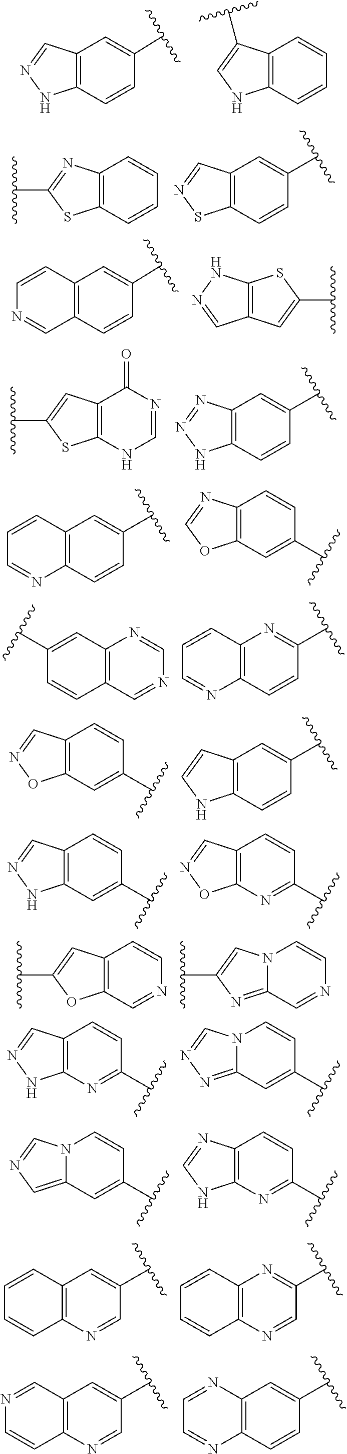

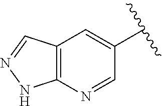 [SMILES: *c1cnc2[nH]ncc2c1]

A C or N atom within group R⁸ may be bonded to LINK by a hydrogen atom on that C or N atom being replaced by a bond to LINK. Typically, where group R⁸ is bonded to LINK, this is via a C atom on R⁸. Preferably, a C or N atom within group R⁸ is bonded to LINK such that a hydrogen atom on that C or N atom is replaced by a bond to LINK. Preferably, the C or N atom that is bonded to LINK is a C or N atom of the heteroaryl ring of R⁸ or a C or N atom of the heteroaryl, cycloalkyl, heterocyclyl or phenyl ring of R¹⁰. More preferably, the C or N atom that is bonded to LINK is a C or N atom of the heteroaryl ring of R.

In addition to any bond to LINK (where present) R⁸ is unsubstituted or substituted by one, two or three substituents, for instance one or two substituents. Typically, each substituent is independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy. More typically, each substituent is independently selected from fluoro, chloro, methoxy, R¹⁰, —(CH₂)—R¹⁰, ═O, —CN, and C₁₋₂ alkyl which is itself unsubstituted or substituted by one or two groups selected from selected from halo (e.g. fluoro) and methoxy. Preferably, each substituent is independently selected from fluoro, R¹⁰ and methyl. Most preferably, each substituent is independently selected from fluoro and methyl. R¹⁰ is as defined herein.

Typically, R¹⁰ is a group selected from a 5- to 6-membered heteroaryl ring, a 3- to 6-membered cycloalkyl ring, a 4- to 5-membered heterocyclyl ring, and a phenyl ring. The cycloalkyl, heteroaryl and heterocyclyl rings are as defined herein. Preferably, R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl.

Typically, in addition to any bond to LINK (where present), R¹⁰ is unsubstituted or substituted by one or two substituents independently selected from fluoro, methyl and methoxy. Preferably, R¹⁰ is unsubstituted.

Typically, therefore, R⁸ is an 8- to 10-membered heteroaryl ring; the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁. 2 alkoxy; wherein R¹⁰ is a group selected from a 5- to 6-membered heteroaryl ring, a 3- to 6-membered cycloalkyl ring, a 4- to 5-membered heterocyclyl ring, and a phenyl ring; and R¹⁰ is unsubstituted or substituted by one or two substituents independently selected from fluoro, methyl and methoxy. Typically, where a C or N atom within group R⁸ is bonded to LINK, the C or N atom that is bonded to LINK is a C or N atom of the heteroaryl ring of R⁸ or a C or N atom of the heteroaryl, cycloalkyl, heterocyclyl or phenyl ring of R¹⁰.

Preferably, R⁸ is an 8- to 10-membered heteroaryl ring containing one, two or three N atoms, for example an indazolyl group; the group R⁸ is unsubstituted or substituted by one or two substituents, each independently selected from fluoro, R¹⁰ and methyl; R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl; and R¹⁰ is unsubstituted.

Preferably, a C or N atom within group R⁸ is bonded to LINK, wherein the C or N atom that is bonded to LINK is a C or N atom of the heteroaryl ring of R.

In a typical embodiment of formula (I), the bicyclic structure formed by rings A and B has the structure:

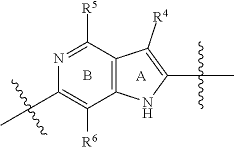

and:
ring Hy is a 5- to 6-membered heterocyclyl ring containing X and at least one N atom, for example a pyrrolidinyl, piperidinyl, morpholinyl or diazinanyl ring; wherein the N atom of ring Hy which is substituted by R¹ is adjacent to the C atom of ring Hy that is bonded to ring A;
R¹ is H or C₁₋₂ alkyl which is unsubstituted or substituted with one C₁₋₂ alkoxy or one, two or three halo, preferably with one C₁₋₂ alkoxy;
R² is H or methyl;
X is a bond, —N(Me)-, O or —CH₂—;
ring Hy is further unsubstituted or substituted by one or two R³; wherein each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring;
R⁴ is selected from H, halo (e.g. fluoro or chloro), methoxy, methyl and trifluoromethyl;
R⁵ and R⁶ are independently selected from H, halo (e.g. fluoro or chloro), methoxy, methyl and trifluoromethyl;
the C atom of L is bonded to R⁸, and the N atom of L is bonded to ring B;
R⁸ is an 8- to 10-membered heteroaryl ring; wherein the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from halo and C₁₋₂ alkoxy; and
R¹⁰ is a group selected from a 5- to 6-membered heteroaryl ring, a 3- to 6-membered cycloalkyl ring, a 4- to 5-membered heterocyclyl ring, and a phenyl ring; wherein R¹⁰ is unsubstituted or substituted by one or two substituents independently selected from fluoro, methyl and methoxy;
wherein M is bonded to LINK via a C or N atom within group R⁸ or ring Hy such that a hydrogen atom on the C or N atom within group R⁸ or ring Hy is replaced with a bond to LINK.

In a preferred embodiment of formula (I), the bicyclic structure formed by rings A and B has the structure:

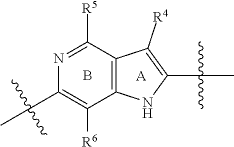

and:
ring Hy is a pyrrolidinyl ring; wherein the N atom of ring Hy which is substituted by R¹ is adjacent to the C atom of ring Hy that is bonded to ring A; R^{1′} is H, methyl, ethyl or methoxyethyl, more preferably methyl; R² is H; and ring Hy is substituted by no R³ groups; either R⁴, R¹ and R⁶ are H, or one of R⁴, R⁵ and R⁶ is selected from halo (e.g. fluoro or chloro), CN, methoxy, methyl and trifluoromethyl, and the rest are H, with the proviso that R¹ and R⁶ are not CN;
the C atom of L is bonded to R⁸, and the N atom of L is bonded to ring B;
R⁸ is an 8- to 10-membered heteroaryl ring containing one, two or three N atoms, for example an indazolyl group; the group R⁸ is unsubstituted or substituted by one or two substituents, each independently selected from fluoro, R¹⁰ and methyl; R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl; and R¹⁰ is unsubstituted;
wherein M is bonded to LINK via a C or N atom within group R⁸ or ring Hy such that a hydrogen atom on the C or N atom within group R⁸ or ring Hy is replaced with a bond to LINK.

In a particular embodiment of the compounds of the invention, the MLLT1 and/or MLLT3 binder group M is a monovalent moiety of formula (II):

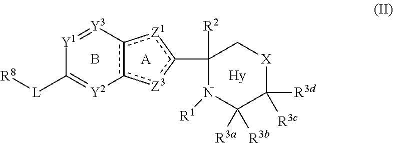

wherein:
Z¹, Z³, Y¹, Y², Y³, R¹, R², R⁸, X and L are as defined herein;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)-, O, S, —S(O)₂— or —S(O)(NR¹¹)-then neither R^{3c} nor R^{3d} are halo;
wherein at least two of R^{3a}, R^{3b}, R^{3c} and R^{3d} are H; and
wherein M is bonded to LINK via a C or N atom within group R⁸ or ring Hy such that a hydrogen atom on the C or N atom within group R⁸ or ring Hy is replaced with a bond to LINK.

Typically, in formula (II): R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is —N(R¹¹)—, O, S, —S(O)₂— or —S(O)(NR¹¹)—, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring); wherein at least two of R^{3a}, R^{3b}R^{3c} and R^{3d} are H. In an alternative typical embodiment: R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₃₋₆ cycloalkyl ring (in particular a cyclopropyl or cyclopentyl ring), with the proviso that when X is NR¹¹, O, or S, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring); wherein at least two of R^{3a}, R^{3b}, R^{3c} and R^{3d} are H.

Optionally, one of R^{3a}, R^{3b}, R^{3c} and R^{3d} is replaced with a single bond to LINK.

Preferably, in formula (II), R^{3a}, R^{3b}, R^{3c} and R^{3d} are all H, or one of R^{3a}, R^{3b}, R^{3c} and R^{3d} is methyl, ethyl or t-butyl and the rest are H, or R^{3c} and R^{3d} are methyl and the rest are H. More preferably, R^{3a}, R^{3b}, R^{3c} and R^{3d} are all H.

In a typical embodiment of formula (II), the bicyclic structure formed by rings A and B has the structure:

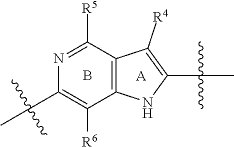

and:
R¹ is H or C₁₋₂ alkyl which is unsubstituted or substituted with one C₁₋₂ alkoxy or one, two or three halo, preferably with one C₁₋₂ alkoxy;
R² is H or methyl;
X is a bond, —N(Me)-, O or —CH₂—;
R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is NR¹¹ or O, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring);
wherein at least two of R^{3a}, R^{3b}, R^{3c} and R^{3d} are H;
R⁴ is selected from H, CN, halo (e.g. fluoro or chloro), methoxy, methyl and trifluoromethyl;
R⁵ and R⁶ are independently selected from H, halo (e.g. fluoro or chloro), methoxy, methyl and trifluoromethyl;
the C atom of L is bonded to R⁸, and the N atom of L is bonded to ring B;
R⁸ is an 8- to 10-membered heteroaryl ring; wherein the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy; and
R¹⁰ is a group selected from a 5- to 6-membered heteroaryl ring, a 3- to 6-membered cycloalkyl ring, a 4- to 5-membered heterocyclyl ring, and a phenyl ring; wherein R¹⁰ is unsubstituted or substituted by one or two substituents independently selected from fluoro, methyl and methoxy;
wherein M is bonded to LINK via a C or N atom within group R⁸ or ring Hy such that a hydrogen atom on the C or N atom within group R⁸ or ring Hy is replaced with a bond to LINK.

In a preferred embodiment of formula (II), the bicyclic structure formed by rings A and B has the structure:

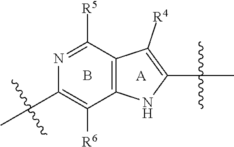

and:
X is absent;
R¹ is H, methyl, ethyl or methoxyethyl, more preferably methyl;
R² is H;
R^{3a}, R^{3b}, R^{3c} and R^{3d} are all H;
either R⁴, R⁵, and R⁶, are H, or one of R⁴, R⁵, and R⁶ is selected from fluoro, CN, methoxy, methyl and trifluoromethyl, and the rest are H, with the proviso that R⁵ and R⁶ are not H;
the C atom of L is bonded to R⁸, and the N atom of L is bonded to ring B;
R⁸ is an 8- to 10-membered heteroaryl ring containing one, two or three N atoms, for example an indazolyl group; the group R⁸ is unsubstituted or substituted by one or two substituents, each independently selected from fluoro, R¹⁰ and methyl; R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl; and R¹⁰ is unsubstituted; and
wherein M is bonded to LINK via a C or N atom within group R⁸ or ring Hy such that a hydrogen atom on the C or N atom within group R⁸ or ring Hy is replaced with a bond to LINK.

In a particularly preferred embodiment of the compounds of the invention, the MLLT1 and/or MLLT3 binder M is a monovalent moiety of formula (III):

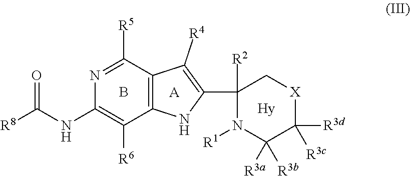

wherein R¹, R², R^{3a}, R^{3b}, R^{3c}, R^{3d}, X, R⁴, R⁵, R⁶ and R⁸ are as defined herein.

Typically, in the compounds of formula (III):
R¹ is H or C₁₋₄ alkyl which is itself unsubstituted or substituted with C₁₋₄alkoxy;
R² is H or methyl;
R^{3a} and R^{3b} are independently selected from H, C₁₋₄alkyl, and C₁₋₄alkoxy, or R^{3a} and R^{3b} form, together with the C atom to which they are attached, a C₃₋₆ cycloalkyl ring,
R^{3c} and R^{3d} are independently selected from H, C₁₋₄alkyl, halo and C₁₋₄alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring, with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo C₁₋₄ alkoxy, and C₁₋₄ alkyl which is itself unsubstituted or substituted by one, two or three halo;
R⁵ and R⁶ are independently selected from H, halo C₁₋₄ alkoxy, and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy; and
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
wherein M is bonded to LINK via a C or N atom within group R⁸ or ring Hy (as described herein) such that a hydrogen atom on the C or N atom within group R⁸ or ring Hy is replaced with a bond to LINK.

Typically, in formula (III), R¹ is H, or C₁₋₄ alkyl which is itself unsubstituted or substituted with one C₁₋₄ alkoxy or one, two or three halo, preferably with one C₁₋₄ alkoxy, for example R¹ may be H or C₁₋₂ alkyl which is unsubstituted or substituted with one C₁₋₂ alkoxy or one, two or three halo, preferably with one C₁₋₂ alkoxy. Preferably, R¹ is H, methyl, ethyl or methoxyethyl. Most preferably, R¹ is methyl.

In formula (III), R² is H or methyl. Preferably, R² is H.

Typically, in formula (III), X is a bond, —N(R¹¹)-, O or —C(R¹¹)₂—. Preferably, X is a bond, —N(Me)-, O or —CH₂—, most preferably X is a bond.

Typically, in formula (III), each R¹¹ is independently selected from H and methyl. When X is —N(R¹¹)—, R¹¹ is preferably methyl. When X is —C(R¹¹)₂—, one R¹¹ is optionally replaced with a single bond to LINK and the other R¹¹ is H. When X is —C(R¹¹)₂—, each R¹¹ is preferably H.

Typically, in formula (III), R^{3a} and R^{3b} are independently selected from H, C₁₋₄ alkyl, halo and C₁₋₄ alkoxy, in particular from H and methyl; R^{3c} and R^{3d} are independently selected from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is NR¹¹ or O, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring); wherein at least two of R^{3a}, R^{3b}, R^{3c} and R^{3d} are H.

Preferably, in formula (III), R^{3a}, R^{3b}, R^{3c} and R^{3d} are all H. Alternatively, one of R^{3a}, R^{3b}, R^{3c} and R^{3d} is methyl, ethyl or t-butyl, and the rest are H, or R^{3c} and R^{3d} are methyl and the rest are H.

In all embodiments of formula (III) described herein, when X is —N(R¹¹)— or O then neither R^{3c} nor R^{3d} are halo (e.g. fluoro).

Typically, in formula (III), R⁴ is selected from H, CN, halo, C₁₋₄ alkoxy, and C₁₋₄ alkyl which is itself unsubstituted or substituted by one, two or three halo. Preferably, R⁴ is selected from H, CN, F, Cl, Me and CF₃.

Typically, in formula (III), R⁵ is selected from H and C₁₋₄ alkyl. Preferably, R⁵ is selected from H and methyl, more preferably H.

Typically, in formula (III), R⁶ is H.

Typically, therefore, in formula (III):
R⁴ is selected from H, CN, halo, C₁₋₄ alkoxy, and C₁₋₄ alkyl which is itself unsubstituted or substituted by one, two or three halo, and R⁵ and R⁶ are H; or
R⁵ is selected from H and C₁₋₄ alkyl, and R⁴ and R⁶ when present are H; or
R⁴, R⁵, and R⁶ when present are H.

Preferably, in in formula (III):
R⁴ is selected from selected from H, CN, F, Cl, Me and CF₃, and R⁵ and R⁶ are H; or
R⁵ is selected from H and methyl, and R⁴ and R⁶ when present are H; or
R⁴, R⁵, and R⁶ when present are H.

In one alternative preferred embodiment, R⁵ and R⁶ are as defined herein and R⁴ is selected from halo, CN, C₁₋₄ alkoxy, and C₁₋₄ alkyl which is itself unsubstituted or substituted by one, two or three halo. More preferably, R⁵ and R⁶ are as defined herein and R⁴ is selected from halo, CN, C₁₋₄ alkyl and CF₃. More preferably, R⁵ and R⁶ are as defined herein and R⁴ is selected from halo, and CF₃. Most preferably, R¹ and R⁶ are as defined herein and R⁴ is selected from F, Cl, and CF₃.

In one preferred embodiment of formula (III), R⁸ is a group selected from indazolyl, thieno[2,3-c]pyrazolyl, imidazo[4,5-b]pyridinyl, pyrazolo[3,4-b]pyridinyl, furo[2,3-c]pyridinyl, indolyl, benzoxazolyl, benzothiazolyl, [1,2,4]triazolo[4,3-a]pyridinyl, thieno[2,3-d]pyrimidinyl, 1,2,3-benzotriazolyl, imidazo[1,5-a]pyridinyl, imidazo[1,2-a]pyrazinyl, oxazolo[5,4-b]pyridinyl, quinolinyl, naphthyridinyl, isoquinolinyl, quinazolinyl, and quinoxalinyl. Preferably, BW is an 8 to 10-membered heteroaryl ring containing one, two or three N atoms. Most preferably, R⁸ is an indazolyl group.

Typically, in formula (III), R⁸ is a group selected from the following structures. For the avoidance of doubt, the following structures may be unsubstituted or substituted by one, two or three substituents as defined herein.

### [See Below]

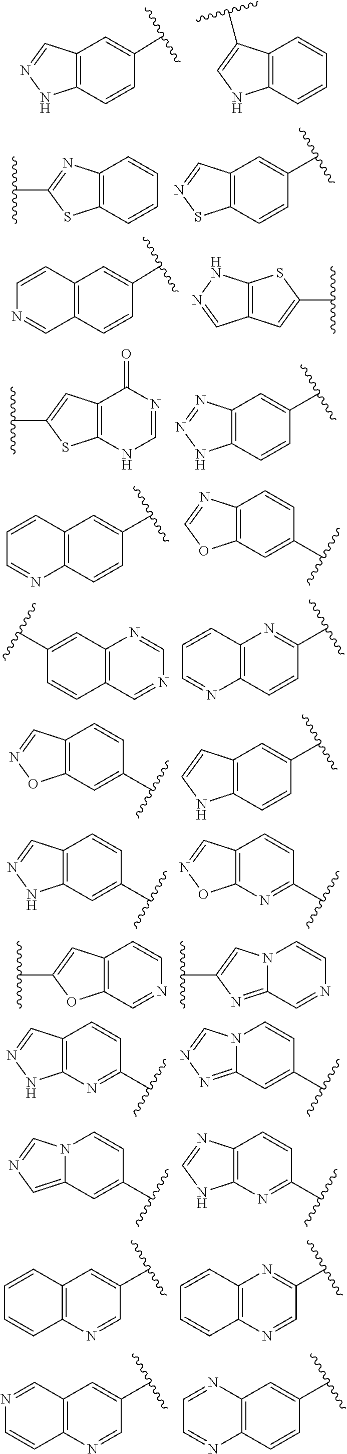

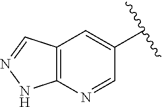 [SMILES: *c1cnc2[nH]ncc2c1]

Preferably, R⁸ is an indazolyl group.

Typically, in formula (III), R⁸ is unsubstituted or substituted by one, two or three substituents, for instance one or two substituents, independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy. More typically, each substituent is independently selected from fluoro, chloro, methoxy, R¹⁰, —(CH₂)—R¹⁰, ═O, —CN, and C₁₋₂ alkyl which is itself unsubstituted or substituted by one or two groups selected from selected from halo (e.g. fluoro) and methoxy.

Preferably, each substituent is independently selected from fluoro, R¹⁰ and methyl. Most preferably, each substituent is independently selected from fluoro and methyl. R¹⁰ is as defined herein.

Typically, in formula (III), R¹⁰ is a group selected from a 5- to 6-membered heteroaryl ring, a 3 to 6-membered cycloalkyl ring, a 4- to 5-membered heterocyclyl ring, and a phenyl ring. The aryl, heteroaryl and heterocyclyl rings are as defined herein. Preferably, R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl.

Typically, in in formula (III), R¹⁰ is unsubstituted or substituted by one or two substituents independently selected from fluoro, methyl and methoxy. Preferably, R¹⁰ is unsubstituted.

In typical embodiments of formula (III):
R¹ is H or C₁₋₂ alkyl which is unsubstituted or substituted with one C₁₋₂ alkoxy or one, two or three halo, preferably with one C₁₋₂ alkoxy;
R² is H or methyl;
X is a bond, —N(Me)-O or —C(H)₂—;
R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is NR¹¹ or O, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring);
wherein at least two of R^{3a}, R^{3b}, R^{3c} and R^{3d} are H;
one of the following options applies: R⁴ is selected from H, CN, halo, C₁₋₄ alkoxy, and C₁₋₄ alkyl which is itself unsubstituted or substituted by one, two or three halo, and R⁵ and R⁶ are H; or R⁵ is selected from H and C₁₋₄ alkyl, and R⁴ and R when present are H; or
R⁴, R⁵, and R when present are H;
the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy; and
R¹⁰ is a group selected from a 5- to 6-membered heteroaryl ring, a 3- to 6-membered cycloalkyl ring, a 4- to 5-membered heterocyclyl ring, and a phenyl ring; wherein R¹⁰ is unsubstituted or substituted by one or two substituents independently selected from fluoro, methyl and methoxy; and R⁸ is a group selected from the following structures:

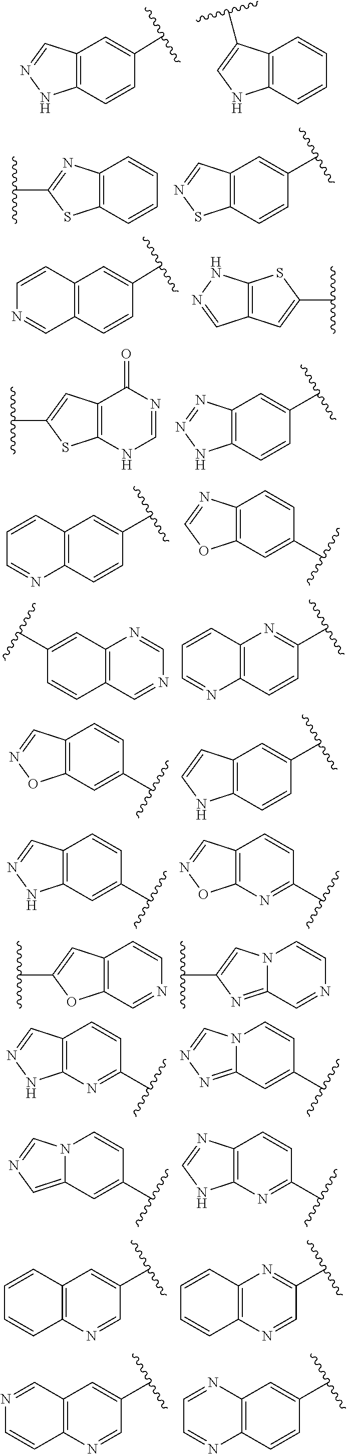

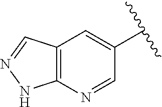

wherein M is bonded to LINK via a C or N atom within group R⁸ or ring Hy such that a hydrogen atom on the C or N atom within group R⁸ or ring Hy is replaced with a bond to LINK.

In preferred embodiments of formula (III),
X is absent;
R¹ is H, methyl, ethyl or methoxyethyl, more preferably methyl;
R² is H;
R^{3a}, R^{3b}, R^{3c} and R^{3d} are all H;
R⁵ and R⁶ are H, and R⁴ is selected from H, CN, F, Cl, Me and CF₃, preferably H or Cl, more preferably H;
R⁸ is an 8- to 10-membered heteroaryl ring containing one, two or three N atoms, for example an indazolyl group; the group R⁸ is unsubstituted or substituted by one or two substituents, each independently selected from fluoro, R¹⁰ and methyl;
R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, preferably pyrazolyl; and R¹⁰ is unsubstituted;
wherein M is bonded to LINK via a C or N atom within group R⁸ or ring Hy such that a hydrogen atom on the C or N atom within group R⁸ or ring Hy is replaced with a bond to LINK.

In a most preferred embodiment of the compounds of the invention, the MLLT1 and/or MLLT3 binder M is a monovalent moiety of formula (IV):

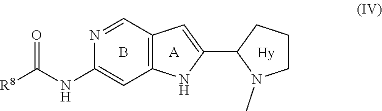

wherein R⁸ is as defined herein. In particular in formula (IV), the group R⁸ may be unsubstituted or substituted by one, two or three substituents independently selected from R¹⁰, halo and C₁₋₄ alkyl;
wherein M is bonded to LINK via a C or N atom within group R⁸ such that a hydrogen atom on the C or N atom within group R⁸ is replaced with a bond to LINK.

Typically, in formula (IV), R⁸ is a group selected from indazolyl, thieno[2,3-c]pyrazolyl, imidazo[4,5-b]pyridinyl, pyrazolo[3,4-b]pyridinyl, furo[2,3-c]pyridinyl, indolyl, benzoxazolyl, benzothiazolyl, [1,2,4]triazolo[4,3-a]pyridinyl, thieno[2,3-d]pyrimidinyl, 1,2,3-benzotriazolyl, imidazo[1,5-a]pyridinyl, imidazo[1,2-a]pyrazinyl, oxazolo[5,4-b]pyridinyl, quinolinyl, naphthyridinyl, isoquinolinyl, quinazolinyl, and quinoxalinyl.

More typically, in formula (IV), R⁸ is a group selected from the following structures. For the avoidance of doubt, the following structures may be unsubstituted or substituted by one, two or three substituents as defined herein.

### [See Below]

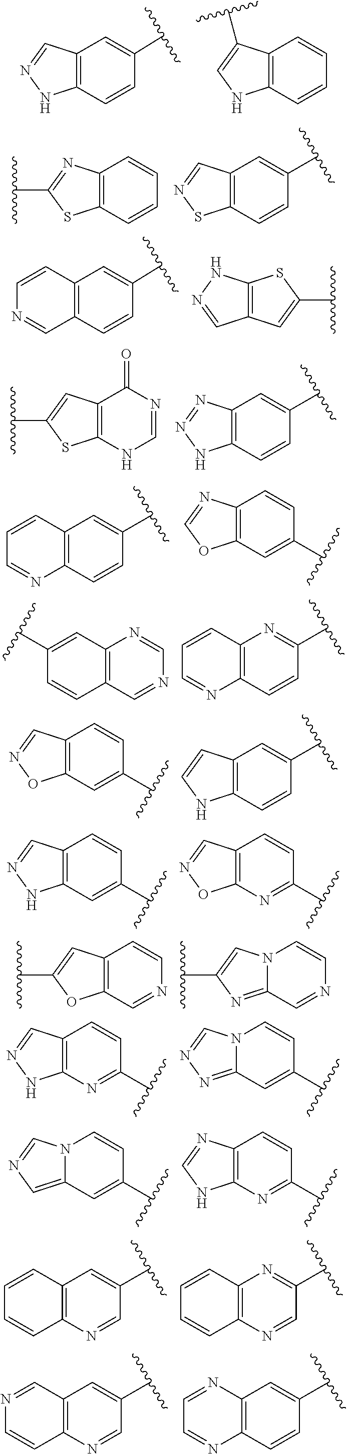

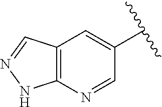 [SMILES: *c1cnc2[nH]ncc2c1]

Preferably, R⁸ is an indazolyl group.

Typically, in formula (IV), R⁸ is unsubstituted or substituted by one, two or three substituents, for instance one or two substituents, independently selected from halo and C₁₋₄ alkyl. Preferably, each substituent is independently selected from fluoro and methyl.

In a particularly preferred embodiment, in formula (IV), R⁸ is selected from one of the following structures:

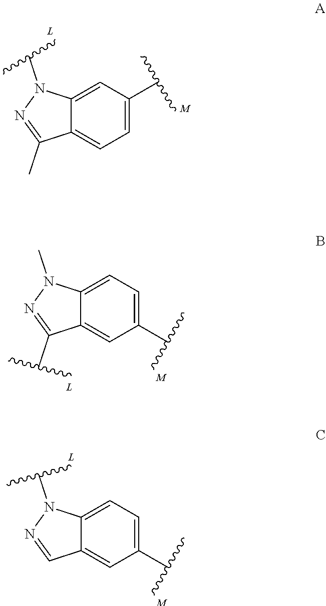

wherein:

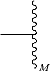

represents the point of attachment of R⁸ to the rest of moiety M; and

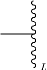

represents the point of attachment of R⁸ to LINK.

For the avoidance of doubt, the group R⁸ is further unsubstituted or substituted as described above. Preferably, the group R⁸ is further unsubstituted or substituted by one or two substituents independently selected from fluorine and methyl, for instance fluorine. For example, the group R⁸ may be selected from one of the following structures:

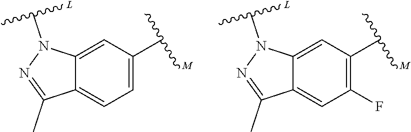

wherein:

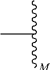

represents the point of attachment of R⁸ to the rest of moiety M; and

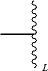

represents the point of attachment of R⁸ to LINK.

For the avoidance of doubt, R⁸ may be selected from any one of the above structures defined herein (including structures A, B and C) in any of formulae (I), (II) and (III).

In another preferred embodiment of the compounds of the invention, the MLLT1 and/or MLLT3 binder M is a monovalent moiety:

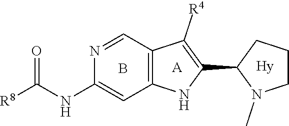

wherein R and R are as defined herein.

In some of these preferred embodiments, R⁴ is selected from H and halo (such as fluoro or chloro). In one such embodiment, R⁴ is H. In another such embodiment, R⁴ is halo (such as fluoro or chloro).

### E3 Ubiquitin Ligase Binding Moieties

The presence of the E3 ubiquitin ligase binding moiety U in the compound of the invention means that the compound is a so-called “PROTAC”.

The term PROTAC is an acronym for proteolysis targeting chimera. In general, PROTACs are, as is known in the art, heterobifunctional molecules that comprise two active moieties attached covalently by a linker group. In a PROTAC, the first active moiety (the MLLT1 and/or MLLT3 binder M in the compound of the present invention) binds to a target protein that is intended for degradation (target proteins for the compound of the present invention are MLLT1 and/or MLLT3). The second active moiety (U in the compound of the present invention) is capable of binding to an E3 ubiquitin ligase, thereby inducing selective intracellular proteolysis. Recruitment of the E3 ligase to the target protein results in ubiquitination and subsequent degradation of the target protein by the proteasome.

E3 ubiquitin ligase moities are known in the art. Substantially any such moiety can be used. The sole limitation on the moiety is that it be capable of binding to an E3 ubiquitin ligase. Those skilled in the art would appreciate that entirely routine laboratory methods can be used to determine whether a given substance binds to an E3 ubiquitin ligase (including but not limited to any of those disclosed specifically herein). Thus, those skilled in the art would have no difficulty in identifying E3 ubiquitin ligase moieties, nor in establishing whether any existing chemical moiety falls within the bounds of this definition. In certain embodiments, the moiety shows activity or binds to the E3 ubiquitin ligase with an IC₅₀ of less than about 200 mM. The IC₅₀ can be determined according to any method known in the art, e.g., a fluorescent polarization assay.

Merely by way of example of the extensive disclosure in the field concerning PROTACs, and hence E3 ubiquitin ligase binding moieties, reference can be made to Gu et al. (BioEssays 2018, 40, 1700247), Sun et al. (Signal Transduction and Targeted Therapy (2019) 4:64), WO 2020/041331, and WO 2019/140003, the contents of all of which are herein incorporated by reference in their entireties. Any of the numerous E3 ubiquitin ligase binding moieties disclosed in these documents can be used as an E3 ubiquitin ligase binding moiety in the compounds of the present invention. For the avoidance of doubt, Gu et al. refer to such moieties as ligands to recruit E3 ubiquitin ligase, Sun et al. refer to such moieties as E3 ubiquitin ligase (E3) recruiting ligands, in WO 2020/041331 such moieties are referred to as a “ULM” or (small molecule) E3 ubiquitin ligase binding moiety (that binds an E3 ubiquitin ligase) (and noting that the term “ULM” includes each of “ILM”, “CLM”, “VLM” and “MLM”, any of which can be used in the present compounds), and in WO 2019/140003 such moieties (labelled “B” in WO 2019/140003's formula (I)) are referred to as a ubiquitin ligase ligand/binder.

Further examples of documents disclosing suitable moieties that can be used in the compounds of the present invention are WO2013/106643, US2016/0045607, WO2014187777, US20140356322 and U.S. Pat. No. 9,249,153, US2016/0058872, US2015/0291562 and Winter et al (Science, Jun. 19, 2015, p. 1376), the contents of all of which are herein incorporated by reference in their entireties. Thalidomide, lenalidomide, pomalidomide and analogs thereof are still further examples of suitable moieties.

Examples of E3 ubiquitin ligases include von Hippel-Lindau (VHL) and cereblon (CRBN).

Preferably the E3 ubiquitin ligase binding moiety (U) is capable of binding to CRBN or VHL. Such moieties are referred to herein as CRBN or VHL E3 ubiquitin ligase binding moieties.

In one embodiment, U is a CRBN E3 ubiquitin ligase binding moiety. In another embodiment, U is a VHL E3 ubiquitin ligase binding moiety.

In some embodiments, the E3 ubiquitin ligase binding moiety (U) is:
(a) a CRBN E3 ubiquitin ligase binding moiety of formula (U1):

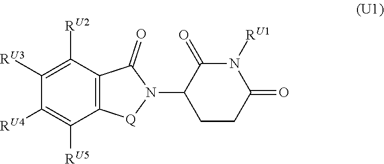

wherein:
R^{U1} is H;
Q is selected from —CH(R^{U6})—, —N(R^{U6})—, —O—, —C(O)—, —NH—CH(R^{U6})—
N═C(R^{U6})—, or —N═N—;
R^{U6} is H or C₁₋₄alkyl;
R^{U2} and R^{U5} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

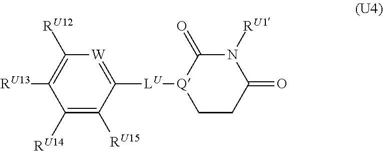

wherein:
R^{U1′} is H;
W is N or CR^{U16};
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U12}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

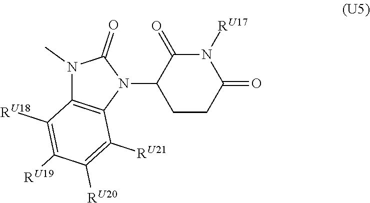

wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

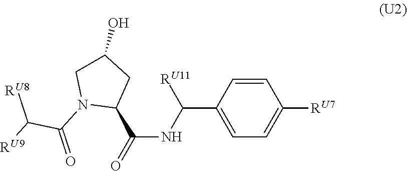

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

For the avoidance of doubt, in formula (U1), when Q is —NH—CH(R^{U6})— or —N═C(R^{U6})—, the N atom of Q is bonded to the phenyl ring in U, and the C atom of Q (not including any C atom that may be in R^{U6}) is bonded to the N atom in U that is adjacent to group Q.

In some embodiments, the E3 ubiquitin ligase binding moiety (U) is preferably a CRBN E3 ubiquitin ligase binding moiety of formula (U1).

Typically, in formula (U1), Q is selected from —CH(R^{U6})— and —C(O)—. Preferably, Q is selected from —CH₂— and —C(O)—. Most preferably, Q is —CH₂—.

Typically, in formula (U1), R^{U6} is H or methyl. Preferably, R^{U6} is H.

Typically, in formula (U1), R^{U2} and R^{U}s are each independently selected from H, fluoro, methyl, NH₂, NO₂, OH, COOH, CN, CF₃, and a single bond to LINK. Preferably, R^{U2} and R^{U5} are each independently selected from H and a single bond to LINK. Most preferably, R^{U2} is H, and R^{U5} is a single bond to LINK.

Typically, in formula (U1), R^{U3} and R^{U4} are each independently selected from H, fluoro, methyl, NH₂, NO₂, OH, COOH, CN, CF₃ and a single bond to LINK. Preferably, R^{U3} and R^{U4} are each independently selected from H and a single bond to LINK.

Typically, in formula (U1), two or three of R^{U2}, R^{U3}, R^{U4} and R^{U5} are H. Preferably, three of R^{U2}, R^{U3}, R^{U4} and R^{U5} are H. More preferably, R^{U2} and R^{U3} are H, one of R^{U4} and R^{U5} is a single bond to LINK, and the other of R^{U4} and R^{U5} is H. Most preferably, R^{U2}, R^{U3} and R^{U4} are H, and R^{U5} is a single bond to LINK.

Typically, therefore, in formula (U1):
Q is selected from —CH(R^{U6})— and —C(O)—;
R^{U6} is H or methyl;
R^{U2} and R^{U5} are each independently selected from H, fluoro, methyl, NH₂, NO₂, OH, COOH, CN, CF₃, and a single bond to LINK;
R^{U3} and R^{U4} are each independently selected from H, fluoro, methyl, NH₂, NO₂, OH, COOH, CN, CF₃ and a single bond to LINK; and
two or three of R^{U2}, R^{U3}, R^{U4} and R^{U5} are H.

Preferably, in formula (U1):
Q is selected from —CH₂— and —C(O)—;
R^{U6} is H;
one of R^{U2}, R^{U3}, R^{U4} and R^{U5} is a single bond to LINK, and the rest are H, for instance wherein R^{U2} and R^{U3} are H, one of R^{U4} and R^{U5} is a single bond to LINK, and the other of R^{U4} and R^{U5} is H.

Most preferably, in formula (U1):
Q is —CH₂—;
R^{U6} is H;
R^{U2}, R^{U3} and R^{U4} are H, and R^{U5} is a single bond to LINK.

Typically, in formula (U4), Q′ is N and L^{U} is a single bond, or Q′ is CH and L^{U} is —C(O)N(H)—. Preferably, Q′ is N and L^{U} is a single bond.

Typically, in formula (U4), when L^{U} is —C(O)N(H)—, then the C atom of L^{U} is bonded to phenyl, and the N atom of L^{U} is bonded to Q′.

Typically, in formula (U4), R^{U12}, R^{U13} and R^{U14} are each independently selected from H, fluoro, methyl, NH₂, NO₂, OH, COOH, CN, CF₃, and a single bond to LINK (wherein one and only one of R^{U12}, R^{U13} and R^{U14} is a single bond to LINK). Preferably, one of R^{U12}, R^{U13} and R^{U14} is a single bond to LINK, one of R^{U12}, R^{U13} and R^{U14} is H and the other of R^{U12}, R^{U13} and R^{U14} is selected from H, fluoro, methyl, NH₂, NO₂, OH, COOH, CN, CF₃, preferably from H, fluoro and methyl. More preferably, one of R^{U12}, R^{U13} and R^{U14} is a single bond to LINK and the other two are H, for instance wherein R^{U12} and R^{U14} are H, and R^{U13} is a single bond to LINK.

Typically, in formula (U4), R^{U15} and R^{U16} (when present) are each independently selected from H, fluoro, methyl, NH₂, NO₂, OH, COOH, CN and CF₃. Preferably, R^{U15} and R^{U16} (when present) are each independently selected from H, F and Me.

In one embodiment of formula (U4), R^{U15} and R^{U16} (when present) are H, and two of R^{U12}, R^{U13} and R^{U14} are H. Preferably, R^{U13}, R^{U14} and R^{U15} are H, R^{U12} is a single bond to LINK, and R^{U16} (when present) is selected from H, F and Me.

Typically, therefore, in formula (U4):
Q′ is N and L^{U} is a single bond, or Q′ is CH and L^{U} is —C(O)N(H)—;
R^{U12}, R^{U13} and R^{U14} are each independently selected from H, fluoro, methyl, NH₂, NO₂, OH, COOH, CN, CF₃, and a single bond to LINK; and
R^{U15} and R^{U16} (when present) are each independently selected from H, fluoro, methyl, NH₂, NO₂, OH, COOH, CN and CF₃;
wherein one and only one of R^{U12}, R^{U13} and R^{U14} is a single bond to LINK.

Preferably, in formula (U4):
Q′ is N and L^{U} is a single bond;
one of R^{U12}, R^{U13} and R^{U14} is a single bond to LINK and the other two are H, for instance wherein R^{U12} and R^{U14} are H, and R^{U13} is a single bond to LINK, or R^{U13}, R^{U14} and R^{U15} are H, R^{U12} is a single bond to LINK, and R^{U16} (when present) is selected from H, F and Me.

Typically, in formula (U5), R^{U18}, R^{U19}, R^{U20} and R^{U21} are each independently selected from H, fluoro, methyl, NH₂, NO₂, OH, COOH, CN, CF₃, and a single bond to LINK (wherein one and only one of R^{U18}, R^{U19}, R^{U20} and R^{U21} is a single bond to LINK). Preferably, one of R^{U18}, R^{U19}, R^{U20} and R^{U21} is a single bond to LINK, one or two, preferably two of R^{U18}, R^{U19}, R^{U20} and R^{U21} are H and the other one or two, preferably one of R^{U18}, R^{U19}, R^{U20} and R^{U21} are selected from H, fluoro, methyl, NH₂, NO₂, OH, COOH, CN, CF₃, preferably from H, fluoro and methyl. More preferably, one of R^{U18}, R^{U19}, R^{U20} and R^{U21} is a single bond to LINK and the other three are H, for instance wherein R^{U19}, R^{U20} and R^{U21} are H, and R^{U18} is a single bond to LINK.

Typically, in formula (U2), R^{U7} is a 5- to 6-membered heteroaryl ring. Preferably, R^{U7} is a 5-membered heteroaryl ring containing one S atom and optionally one N atom.

Typically, in formula (U2), R^{U7} is unsubstituted or substituted by C₁₋₄ alkyl. Preferably, R^{U7} is unsubstituted or substituted by methyl.

Typically, in formula (U2), R^{U11} is H or methyl. Preferably, R^{U11} is H.

Typically, in formula (U2), R^{U8} is C₁₋₄ alkyl or phenyl. Preferably, R^{U5} is t-butyl or phenyl, more preferably t-butyl.

Typically, therefore, in formula (U2), R^{U7} is a 5- to 6-membered heteroaryl ring, the group R^{U7} being unsubstituted or substituted by C₁₋₄ alkyl, R^{U11} is H or methyl, and R^{U5} is C₁₋₄ alkyl or phenyl.

Preferably, in formula (U2), R^{U7} is a 5-membered heteroaryl ring containing one S atom and optionally one N atom, the group R^{U7} being unsubstituted or substituted by methyl, R^{U11} is H, and R^{U8} is t-butyl or phenyl, more preferably t-butyl.

In a preferred embodiment, the E3 ubiquitin ligase binding moiety (U) is a VHL E3 ubiquitin ligase binding moiety of formula (U3):

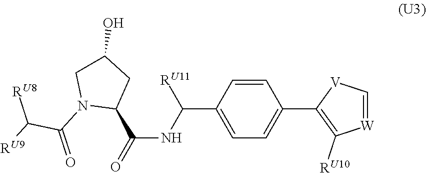

wherein:
V is Sor O;
W is N or CH;
R^{U10} is H or C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

Typically, in formula (U3), V is S.

Typically, in formula (U3), W is N or CH. Preferably, W is N.

Typically, in formula (U3), R^{U10} is H or methyl. Preferably, R^{U10} is methyl.

Typically, in formula (U3), R^{U11} is H or methyl. Preferably, R^{U11} is H.

Typically, in formula (U3), R^{U8} is C₁₋₄ alkyl or phenyl. Preferably, R^{U8} is t-butyl or phenyl, more preferably t-butyl.

Typically, therefore, in formula (U3), V is S, W is N or CH, R^{U10} is H or methyl, R^{U11} is H or methyl, and R^{U8} is C₁₋₄ alkyl or phenyl. Preferably, V is S, W is N, R^{U10} is methyl, R^{U11} is H, R^{U8} is t-butyl or phenyl, more preferably t-butyl.

The moieties U of formulae (U1), (U4) when Q′ is CH and (U5) typically contain at least one chiral centre at the carbon atoms marked with an asterisk as depicted below:

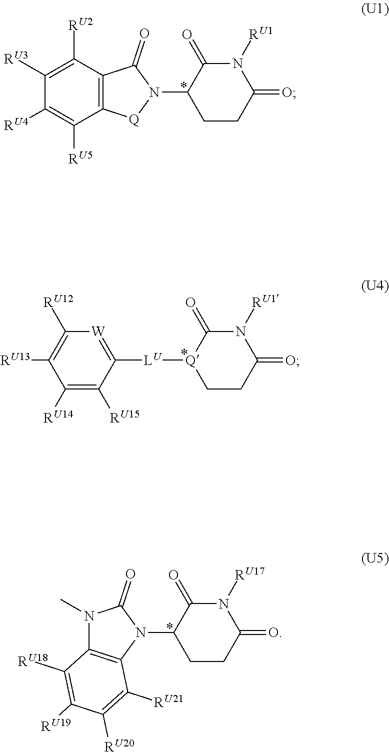

Moiety U may therefore be provided in the form of the R-enantiomer at said carbon atom, in the form of the S-enantiomer at said carbon atom, or in the form of a mixture (for example a 1:1 mixture) of the two enantiomers. A pure enantiomeric form of either the R- or the S-enantiomer may be preferred. In some embodiments, the S-enantiomer at said carbon atom is preferred. In other embodiments, the R-enantiomer at said carbon atom is preferred. The substance or agent described herein may contain at least 50%, preferably at least 60%, 75%, 90% or 95% of a compound which is enantiomerically or diasteriomerically pure at moiety U. Unless otherwise stated, the moiety U may typically be provided in the form of a 1:1 mixture of the two enantiomers.

In some embodiments, the S-enantiomer at said carbon atom is preferred. The preferred stereochemistry at said carbon atom is also depicted by the following illustrative structure of formula (U1):

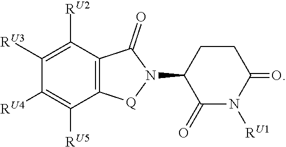

where LINK is attached to moiety U at the R^{U5} position.

In some preferred embodiments, the E3 ubiquitin ligase binding moiety (U) has the following structure:

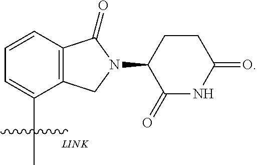 [SMILES: *c1cccc2c1CN([C@H]1CCC(=O)NC1=O)C2=O]

### Linkers

As discussed herein, in the compounds of the invention, the binder of MLLT1 and/or MLLT3 (M) is covalently attached to an E3 ubiquitin ligase binding moiety (U). This covalent attachment is labelled LINK and may be a direct single bond or a divalent chemical linker group that forms covalent bonds both to the binder of MLLT1 and/or MLLT3 (M), and to the E3 ubiquitin ligase binding moiety (U). Typically, LINK is a divalent chemical linker group that forms covalent bonds both to the binder of MLLT1 and/or MLLT3 (M), and to the E3 ubiquitin ligase binding moiety (U).

There is no particular limitation on the nature of LINK in the compounds of the present invention (beyond that the respective active moieties, e.g., U and M, are able to exert their desired function and LINK is capable of covalently attaching them together). Those skilled in the art would recognise that chemical linker groups are routinely used in the construction of bifunctional molecules and would be able routinely to provide appropriate chemical linker groups for attaching particular U and M moieties together. Typically, a chemical linker group for use in the present invention is an organic group.

When LINK is a divalent chemical linker group, it may be represented by formula (L):

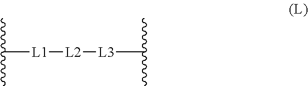

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one or two C₁₋₄ alkyl;
L2 is represented by the formula -(L4)ₘ-;
each L4 is independently a unit of formula:

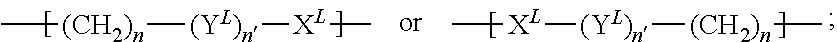

each X^{L} is independently selected from a single bond, —N(R′)—, —O—, —C(O)—, —S—, —SO—, —SO₂—, —C(R″)═C(R″)— and —C≡C—;
each Y^{L} is independently selected from a divalent 3- to 6-membered cycloalkyl ring, a divalent 4- to 7-membered heterocyclyl ring, or one of the following structures:

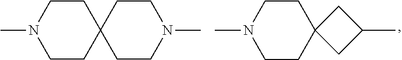

the cycloalkyl and heterocyclyl rings being unsubstituted or substituted by one or two C₁₋₄ alkyl;
n is selected from 0 to 4;
n′ is selected from 0 or 1;
n+n′>1;
m is selected from 1 to 30;
each R′ is independently selected from H and C₁₋₄ alkyl; and
each R″ is independently selected from H and halo.

When m is not 1 (i.e. when there are two or more L4 groups present), then the X^{L} group of any L4 group is not directly bonded to the X^{L} group of any other L4 group that is present.

When the phenylene, cycloalkyl or heterocyclyl rings referred to herein are substituted, they are preferably substituted by one methyl group.

In a preferred embodiment, L1 and L3 are each independently selected from a single bond, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one methyl group.

Typically, in formula (L), L1 is selected from a single bond, branched or straight-chain —(C₁₋₆ alkylene)-, —C(O)N(R′)—, —N(R′)C(O)—, —O—, —S(O₂)N(R′)—, —N(R′)S(O₂)—, and a divalent 4- to 7-membered heterocyclyl ring which is unsubstituted or substituted by one or two C₁₋₄ alkyl. When present, the divalent 4- to 7-membered heterocyclyl ring of L1 typically contains at least one N atom, for instance one or two N atoms. Preferably, when L1 is a divalent 4- to 7-membered heterocyclyl ring, it is a divalent 5- to 6-membered heterocyclyl ring, more preferably a saturated divalent 5- to 6-membered heterocyclyl ring, for instance a divalent moiety of piperidine or diazinane. Preferably, in formula (L), L1 is selected from a single bond, branched or straight-chain —(C₁₋₆ alkylene)-, —C(O)N(H)—, —N(H)C(O)—, and a divalent moiety of piperidine or diazinane which is unsubstituted or substituted by one methyl group. For instance, L1 may be a single bond.

Typically, in formula (L), L3 is selected from a single bond, —N(R′)—, —C(O)N(R′)—, —O—, —N(R′)C(O)—, —S(O₂)N(R′)—, —N(R′)S(O₂)—, ethynylene, —C═C═C—, phenylene and a divalent 4- to 7-membered heterocyclyl ring, the phenylene and heterocyclyl rings being unsubstituted or substituted by one or two C₁₋₄ alkyl. When present, the divalent 4- to 7-membered heterocyclyl ring of L3 typically contains at least one N atom, for instance one or two N atoms. Preferably, when L3 is a divalent 4- to 7-membered heterocyclyl ring, it is a divalent 5- to 6-membered heterocyclyl ring, more preferably a saturated divalent 5- to 6-membered heterocyclyl ring, preferably a divalent moiety of piperidine or diazinane. Preferably, in formula (L), L3 is selected from a single bond, —C(O)N(H)—, —O—, —N(H)C(O)—, ethynylene, —C═C═C—, phenylene and a divalent moiety of piperidine or diazinane, the phenylene and divalent moiety of piperidine or diazinane being unsubstituted or substituted by one methyl group. Most preferably, L3 is selected from a single bond, ethynylene and a divalent moiety of piperidine or diazinane.

L2 is represented by the formula -(L4)ₘ-. In formula (L), each L4 is independently a unit of formula

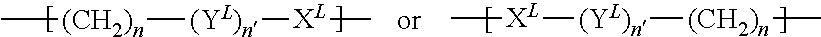

as described above.

Typically, each X^{L} is independently selected from a single bond, —O—, —S—, —C(R″)═C(R″)— and —C≡C—, for example a single bond, —O— and —S—. Preferably, each X^{L} is independently selected from a single bond and —O—.

Typically, each Y^{L} is independently selected from a divalent moiety of piperidine or diazinane, or one of the following structures:

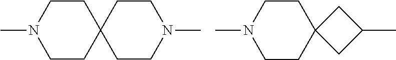

the divalent moiety of piperidine or diazinane being unsubstituted or substituted by one methyl group.

In formula (L), n is selected from 0 to 4; n′ is selected from 0 or 1; and n+n′>1.

In one embodiment, each L4 is the same. Alternatively, L2 may comprise two or more blocks, wherein in each block, each L4 is the same. For instance, L2 may be represented by the formula -(L4′)ₚ-(L4″)_{q}-(L4′″)ᵣ-, wherein L4′, L4″ and L4′″ are selected from those moieties described above for L4, and wherein each L4′ is the same, each L4″ is the same and each L4′″ is the same. Preferably, L4′, L4″ and L4′″ are selected from —CH₂—, —CH₂CH₂O—, —OCH₂CH₂—, —CH₂CH₂S— and —SCH₂CH₂—. More preferably, L4′, L4″ and L4′″ are selected from —CH₂—, —CH₂CH₂O— and —OCH₂CH₂—. p, q and r are integers of from 0 to 30, wherein p+q+r=m.

In one embodiment, each L4 unit is the same. For instance, each L4 in formula (L) may be —CH₂—, each L4 in formula (L) may be —CH₂CH₂O—, each L4 in formula (L) may be —OCH₂CH₂—, each L4 in formula (L) may be —CH₂CH₂S—, or each L4 in formula (L) may be —SCH₂CH₂—. More preferably, each L4 in formula (L) is —CH₂—, each L4 in formula (L) is —CH₂CH₂O— or each L4 in formula (L) is —OCH₂CH₂—. Most preferably, each L4 in formula (L) is —CH₂—.

Typically, in formula (L), m is selected from 1 to 10. For instance, m may be selected from 1 to 8, 1 to 6, 1 to 5, 1 to 4, 1 to 3, 1 to 2, or 1.

Typically, in formula (L), each R′ is independently selected from H and methyl. Preferably, each R′ is H.

Typically, in formula (L), each R¹¹ is independently selected from H, fluoro and chloro. Preferably, each R¹¹ is H and chloro, for example H.

When L2 comprises one or more divalent 4- to 7-membered heterocyclyl rings, typically L2 comprises only one or two, e.g. one divalent 4- to 7-membered heterocyclyl rings. Typically, therefore, L2 comprises none, one or two divalent 4- to 7-membered heterocyclyl rings, for example none or one divalent 4- to 7-membered heterocyclyl rings. When L2 comprises one or more divalent 4- to 7-membered heterocyclyl rings, then in one embodiment L1 and L3 do not contain a divalent 4- to 7-membered heterocyclyl ring.

Typically, therefore, in formula (L):
L1 is selected from a single bond, branched or straight-chain —(C₁₋₆ alkylene)-, —C(O)N(R′)—, —N(R′)C(O)—, —O—, —S(O₂)N(R′)—, —N(R′)S(O₂)—, and a divalent 4- to 7-membered heterocyclyl ring which is unsubstituted or substituted by one or two C₁₋₄ alkyl, the divalent 4- to 7-membered heterocyclyl ring typically containing at least one N atom, for instance one or two N atoms;
L3 is selected from a single bond, —N(R′)—, —C(O)N(R′)—, —O—, —N(R′)C(O)—, —S(O₂)N(R′)—, —N(R′)S(O₂)—, ethynylene, —C═C═C—, phenylene and a divalent 4- to 7-membered heterocyclyl ring, the phenylene and heterocyclyl rings being unsubstituted or substituted by one or two C₁₋₄ alkyl, the divalent 4- to 7-membered heterocyclyl ring typically containing at least one N atom, for instance one or two N atoms;
each X^{L} is independently selected from a single bond, —O—, —S—, —C(R″)═C(R″)— and —C≡C—, for example a single bond, —O— and —S—;
each Y^{L} is independently selected from a divalent moiety of piperidine or diazinane, or one of the following structures:

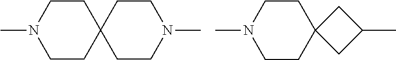

the divalent moiety of piperidine or diazinane being unsubstituted or substituted by one methyl group;
n is selected from 0 to 4; n′ is selected from 0 or 1; and n+n′>1;
m is selected from 1 to 10, preferably from 1 to 4 (for example 1, 2 or 3);
each R′ is independently selected from H and methyl; and
each R″ is independently selected from H, fluro and chloro, for instance H and chloro.

Preferably, in formula (L):
L1 is selected from a single bond, branched or straight-chain —(C₁₋₆ alkylene)-, —C(O)N(H)—, —N(H)C(O)—, and a divalent moiety of piperidine or diazinane which is unsubstituted or substituted by one methyl group;
L3 is selected from a single bond, —C(O)N(H)—, —O—, —N(H)C(O)—, ethynylene, —C═C═C—, phenylene and a divalent moiety of piperidine or diazinane, the phenylene and divalent moiety of piperidine or diazinane being unsubstituted or substituted by one methyl group;
each X^{L} is independently selected from a single bond and —O—;
each Y^{L} is independently selected from a divalent moiety of piperidine or diazinane, or one of the following structures:

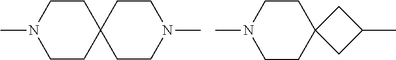

the divalent moiety of piperidine or diazinane being unsubstituted or substituted by one methyl group;
n is selected from 0 to 4; n′ is selected from 0 or 1; and n+n′≥1;
m is selected from 1 to 4 (for example 1, 2 or 3);
each R′ is H; and
each R″ is H or chloro, for instance H.

In a typical embodiment, L2 is —(C₁₋₁₀ alkylene)- or —(C₁₋₁₀ alkylene)-C(O)—, preferably —(C₁₋₁₀ alkylene)-. In this embodiment, L1 is preferably selected from a single bond, —C(O)N(H)—, —N(H)C(O)—, —O—, and a divalent moiety of piperidine or diazinane. In this embodiment, L3 is preferably selected from a single bond, —C(O)N(H)—, —O—, —N(H)C(O)—, ethynylene and a divalent moiety of piperidine or diazinane. More preferably, L1 and L3 are selected from the following options:
L1 is —C(O)N(H)— or —N(H)C(O)—, and L3 is —C(O)N(H)— or —N(H)C(O)—;
L1 is a divalent moiety of piperidine and L3 is a divalent moiety of diazinane;
L1 is —C(O)N(H)— or —N(H)C(O)—, and L3 is a divalent moiety of diazinane;
L1 is a single bond and L3 is a divalent moiety of diazinane;
L1 is a single bond and L3 is a divalent moiety of piperidine;
L1 is a single bond and L3 is —O—;
L1 is a single bond and L3 is ethynylene;
L1 is a single bond and L3 is a single bond; and
L1 is —C(O)N(H)— or —N(H)C(O)—, and L3 is —O—.

In particularly preferred embodiments:
L1 is a divalent moiety of piperidine, L2 is —(C₁₋₁₀ alkylene)- and L3 is a divalent moiety of diazinane;
L1 is a single bond, L2 is —(C₁₋₁₀ alkylene)- and L3 is —O—;
L1 is a single bond, L2 is —(C₁₋₁₀ alkylene)- and L3 is a divalent moiety of piperidine or diazinane;
L1 is a single bond, L2 is —(C₁₋₁₀ alkylene)- and L3 is ethynylene;
L1 is a divalent moiety of piperidine, L2 is —(C₁₋₁₀ alkylene)- and L3 is ethynylene;
L1 is a divalent moiety of diazinane, L2 is —(C₁₋₁₀ alkylene)- and L3 is ethynylene;
L1 is a divalent moiety of diazinane, L2 is —(C₁₋₁₀ alkylene)- and L3 is a divalent moiety of piperidine;
L1 is a single bond, L2 is a divalent moiety of piperidine and L3 is phenylene;
L1 is a divalent moiety of piperidine or diazinane, L2 is —(C₁₋₁₀ alkylene)- and L3 is phenylene;
L1 is —O—, L2 is —(C₁₋₁₀ alkylene)- and L3 is ethynylene;
L1 is a divalent moiety of piperidine, L2 is —(C₁₋₁₀ alkylene)-O— and L3 is a divalent moiety of piperidine.

In one preferred embodiment, LINK has the following structure:

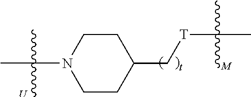

wherein T is —O— or —(CH₂)—, and t is selected from 1 to 10, preferably 1 to 5, more preferably 2.

In another preferred embodiment, L1 is a single bond, L2 is —(C₁₋₁₀ alkylene)- and L3 is ethynylene or a divalent moiety of piperidine (for instance ethynylene), preferably wherein L2 is —(C₃₋₈ alkylene)-, more preferably wherein L2 is —(C₇ alkylene)-.

Alternatively, when LINK is a divalent chemical linker group, it may be represented by formula (L′):

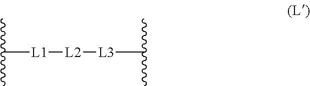

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynyl, —(C₂₋₆ alkenylene)-, a divalent 3- to 6-membered cycloalkyl ring and a divalent 4- to 7-membered heterocyclyl ring;
L2 is represented by the formula -(L4)ₘ-;
each L4 is independently selected from a divalent 3- to 6-membered cycloalkyl ring, a divalent 4- to 7-membered heterocyclyl ring and a unit of formula:

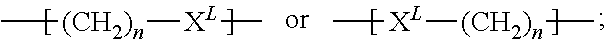

each X^{L} is independently selected from a single bond, —N(R′)—, —O—, —C(O)—, —S—, —SO—, —SO₂—, —C(R″)═C(R″)— and —C(H)—C(H)—;
n is selected from 1 to 4;
m is selected from 1 to 30;
each R′ is independently selected from H and C₁₋₄ alkyl; and
each R″ is independently selected from H and halo.

In one embodiment of formula (L′), L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, —(C₂₋₆ alkenylene)-, ethynyl, a divalent 3- to 6-membered cycloalkyl ring and a divalent 4- to 7-membered heterocyclyl ring;
L2 is represented by the formula -(L4)ₘ-; and
each L4 is independently selected from a divalent 3- to 6-membered cycloalkyl ring, a divalent 4- to 7-membered heterocyclyl ring and a unit of formula:

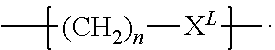

When m is not 1 (i.e. when there are two or more L4 groups present), then the X^{L} group of any L4 group is not directly bonded to the X^{L} group of any other L4 group that is present.

Typically, in formula (L′), L1 is selected from a single bond, branched or straight-chain —(C₁₋₆ alkylene)-, —C(O)N(R′)—, —N(R′)C(O)—, —O—, —S(O₂)N(R′)—, —N(R′)S(O₂)—, and a divalent 4- to 7-membered heterocyclyl ring. When present, the divalent 4- to 7-membered heterocyclyl ring of L1 typically contains at least one N atom, for instance one or two N atoms. Preferably, when L1 is a divalent 4- to 7-membered heterocyclyl ring, it is a divalent 5- to 6-membered heterocyclyl ring, more preferably a saturated divalent 5- to 6-membered heterocyclyl ring, for instance a divalent moiety of piperidine or diazinane. Preferably, in formula (L), L1 is selected from a single bond, branched or straight-chain —(C₁₋₆ alkylene)-, —C(O)N(H)—, —N(H)C(O)—, and a divalent moiety of piperidine or diazinane.

Typically, in formula (L′), L3 is selected from a single bond, —N(R′)—, —C(O)N(R′)—, —O—, —N(R′)C(O)—, —S(O₂)N(R′)—, —N(R′)S(O₂)—, ethynyl and a divalent 4- to 7-membered heterocyclyl ring. When present, the divalent 4- to 7-membered heterocyclyl ring of L3 typically contains at least one N atom, for instance one or two N atoms. Preferably, when L3 is a divalent 4- to 7-membered heterocyclyl ring, it is a divalent 5- to 6-membered heterocyclyl ring, more preferably a saturated divalent 5- to 6-membered heterocyclyl ring, for instance a divalent moiety of piperidine or diazinane, preferably 1,4-diazinane. Preferably, in formula (L), L3 is selected from a single bond, —N(H)—, —C(O)N(H)—, —O—, —N(H)C(O)—, ethynyl and a divalent moiety of piperidine or diazinane.

L2 is represented by the formula -(L4)ₘ-. Typically, in formula (L′), each L4 is independently selected from a divalent 4- to 7-membered heterocyclyl ring and a unit of formula

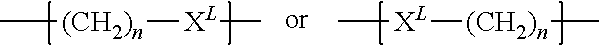

as described above. Typically, each X^{L} is independently selected from a single bond, —O—, —S—, —C(R″)═C(R″)— and —C(H)≡C(H)—, for example a single bond, —O— and —S—, and n is selected from 1 or 2. In one embodiment, each L4 is the same. Alternatively, L2 may comprise two or more blocks, wherein in each block, each L4 is the same. For instance, L2 may be represented by the formula -(L4′)ₚ-(L⁴″)_{q}-(L4′″)ᵣ-, wherein L4′, L4″ and L4′″ are selected from those moieties described above for L4, and wherein each L4′ is the same, each L4″ is the same and each L4′″ is the same. Preferably, L4′, L4″ and L4′″ are selected from —CH₂—, —CH₂CH₂O—, —OCH₂CH₂—, —CH₂CH₂S— and —SCH₂CH₂—. More preferably, L4′, L4″ and L4′″ are selected from —CH₂—, —CH₂CH₂O— and —OCH₂CH₂—. p, q and r are integers of from 0 to 30, wherein p+q+r=m.

In one embodiment of formula (L′), each L4 unit is the same. For instance, each L4 in formula (L′) may be —CH₂—, each L4 in formula (L′) may be —CH₂CH₂O—, each L4 in formula (L′) may be —OCH₂CH₂—, each L4 in formula (L′) may be —CH₂CH₂S—, or each L4 in formula (L′) may be —SCH₂CH₂—. More preferably, each L4 in formula (L′) is —CH₂—, each L4 in formula (L′) is —CH₂CH₂O— or each L4 in formula (L′) is —OCH₂CH₂—. Most preferably, each L4 in formula (L′) is —CH₂—.

Typically, in formula (L′), m is selected from 1 to 10.

Typically, in formula (L′), each R′ is independently selected from H and methyl. Preferably, each R′ is H.

Typically, in formula (L′), each R¹¹ is independently selected from H, fluoro and chloro. Preferably, each R¹¹ is H and chloro, for example H.

When one or more L4 is a divalent 4- to 7-membered heterocyclyl ring, typically only one or two, e.g. one L4 is a divalent 4- to 7-membered heterocyclyl ring. Typically, therefore, none, one or two L4 is a divalent 4- to 7-membered heterocyclyl ring, for example none or one L4 is a divalent 4- to 7-membered heterocyclyl ring.

Typically, therefore, in formula (L′):
L1 is selected from a single bond, branched or straight-chain —(C₁₋₆ alkylene)-, —C(O)N(R′)—, —N(R′)C(O)—, —O—, —S(O₂)N(R′)—, —N(R′)S(O₂)—, and a divalent 4- to 7-membered heterocyclyl ring typically containing at least one N atom, for instance one or two N atoms; L3 is selected from a single bond, —N(R′)—, —C(O)N(R′)—, —O—, —N(R′)C(O)—, —S(O₂)N(R′)—, —N(R′)S(O₂)—, ethynyl and a divalent 4- to 7-membered heterocyclyl ring typically containing at least one N atom, for instance one or two N atoms;
each L4 is independently selected from —CH₂—, —CH₂CH₂O—, —OCH₂CH₂—, —CH₂CH₂S—, —SCH₂CH₂—, and a divalent 4- to 7-membered heterocyclyl ring, wherein when one or more L4 is a divalent 4- to 7-membered heterocyclyl ring, only one or two, e.g. one L4 is a divalent 4- to 7-membered heterocyclyl ring;
m is selected from 1 to 10;
each R′ is independently selected from H and methyl; and
each R″ is independently selected from H, fluro and chloro, for instance H and chloro.

Preferably, in formula (L′):
L1 is selected from a single bond, branched or straight-chain —(C₁₋₆ alkylene)-, —C(O)N(H)—, —N(H)C(O)—, and a divalent moiety of piperidine or diazinane;
L3 is selected from a single bond, —N(H)—, —C(O)N(H)—, —O—, —N(H)C(O)—, ethynyl and a divalent moiety of piperidine or diazinane;
each L4 in formula (L) is —CH₂—, each L4 in formula (L) is —CH₂CH₂O—, each L4 in formula (L) is —OCH₂CH₂—, each L4 in formula (L) is —CH₂CH₂S—, or each L4 in formula (L) is —SCH₂CH₂—, with each L4 preferably being —CH₂—;
m is selected from 1 to 10;
each R′ is H; and
each R″ is H or chloro, for instance H.

In a typical embodiment of formula (L′), L2 is —(C₁₋₁₀ alkylene)- or —(C₁₋₁₀ alkylene)-C(O)—, preferably —(C₁₋₁₀ alkylene)-. In this embodiment, L1 is preferably selected from a single bond, —C(O)N(H)—, —N(H)C(O)—, and a divalent moiety of piperidine or diazinane. In this embodiment, L3 is preferably selected from a single bond, —N(H)—, —C(O)N(H)—, —O—, —N(H)C(O)—, ethynyl and a divalent moiety of piperidine or diazinane. More preferably, L1 and L3 are selected from the following options:
L1 is a single bond and L3 is —N(H)—;
L1 is a divalent moiety of piperidine and L3 is —N(H)—;
L1 is —C(O)N(H)— or —N(H)C(O)—, and L3 is —C(O)N(H)— or —N(H)C(O)—;
L1 is a divalent moiety of piperidine and L3 is a divalent moiety of diazinane;
L1 is —C(O)N(H)— or —N(H)C(O)—, and L3 is a divalent moiety of diazinane;
L1 is a single bond and L3 is a divalent moiety of diazinane;
L1 is a single bond and L3 is a divalent moiety of piperidine;
L1 is a single bond and L3 is —O—;
L1 is a single bond and L3 is ethynyl;
L1 is a single bond and L3 is a single bond; and
L1 is —C(O)N(H)— or —N(H)C(O)—, and L3 is —O—.

For the avoidance of doubt, the skilled person would readily appreciate that each of the definitions of moieties U, M and LINK provided herein may be taken together in any combination to arrive at a definition of the compound of the present invention. As examples that are in no way limiting, definitions of moieties U, M and LINK labelled “typically” may be combined to arrive at a definition of a typical compound of the present invention, and definitions of moieties U, M and LINK labelled “preferably” may be combined to arrive at a definition of a preferred compound of the present invention. However, the skilled person would appreciate that a definition labelled “typically” of one moiety may be combined with a definition labelled “preferably” of another moiety to arrive at a definition of a compound of the present invention.

In preferred compounds of the present invention, M is of formula (III):

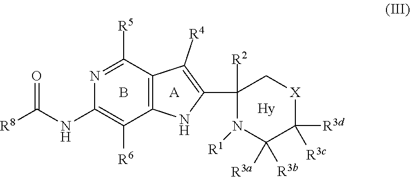

wherein R¹, R², R^{3a}, R^{3b}, R^{3c}, R^{3a}, X, R⁴, R⁵, R⁶ and R⁸ are as defined herein.

Typically, in such preferred compounds:
R¹ is H or C₁₋₄ alkyl which is itself unsubstituted or substituted with C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3a} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring, with the proviso that when X is O or —N(Me)-, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo C₁₋₄ alkoxy, and C₁₋₄ alkyl which is itself unsubstituted or substituted by one, two or three halo;
R⁵ and R⁶ are independently selected from H, halo C₁₋₄ alkoxy, and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy; and
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
wherein M is bonded to LINK via a C or N atom within group R⁸ or ring Hy (as described herein) such that a hydrogen atom on the C or N atom within group R⁸ or ring Hy is replaced with a bond to LINK; and
LINK is (a) a single bond, or (b) a chemical linker group represented by formula (L):

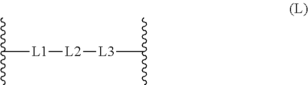

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one or two C₁₋₄ alkyl;
L2 is represented by the formula -(L4)ₘ-;
each L4 is independently a unit of formula:

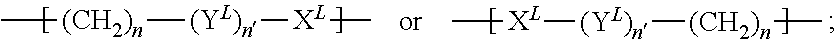

each X^{L} is independently selected from a single bond, —N(R′)—, —O—, —C(O)—, —S—, —SO—, —SO₂—, —C(R″)═C(R″)— and —C≡C—;
each Y^{L} is independently selected from a divalent 3- to 6-membered cycloalkyl ring, a divalent 4- to 7-membered heterocyclyl ring, or one of the following structures:

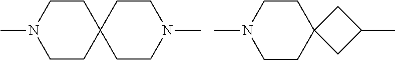

the cycloalkyl and heterocyclyl rings being unsubstituted or substituted by one or two C₁₋₄ alkyl;
n is selected from 0 to 4;
n′ is selected from 0 or 1;
n+n′>1;
m is selected from 1 to 10, preferably from 1 to 4 (for example 1, 2 or 3);
each R′ is independently selected from H and C₁₋₄ alkyl;
each R″ is independently selected from H and halo; U is:
(a) a CRBN E3 ubiquitin ligase binding moiety of formula (U1):

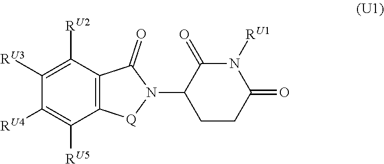

wherein:
R^{U1} is H;
Q is selected from —CH(R^{U6})—, —N(R^{U6})—, —O—, —C(O)—, —NH—CH(R^{U6})—
—N═C(R^{U6})—, or —N═N—;
R^{U6} is H or C₁₋₄alkyl;
R^{U2} and R^{U5} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

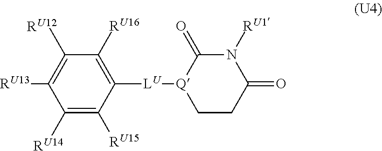

wherein:
R^{U1′} is H;
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U12}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

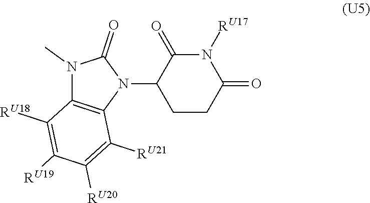

wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

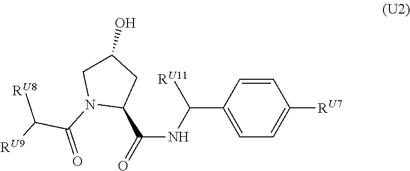

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

Alternatively, in such preferred compounds:
R¹ is H or C₁₋₄ alkyl which is itself unsubstituted or substituted with C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3a} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring, with the proviso that when X is O or —N(Me)-, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo C₁₋₄ alkoxy, and C₁₋₄ alkyl which is itself unsubstituted or substituted by one, two or three halo;
R⁵ and R⁶ are independently selected from H, halo C₁₋₄ alkoxy, and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy; and
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
wherein M is bonded to LINK via a C or N atom within group R⁸ or ring Hy (as described herein) such that a hydrogen atom on the C or N atom within group R⁸ or ring Hy is replaced with a bond to LINK; and
LINK is (a) a single bond, or (b) a chemical linker group represented by formula (L′):

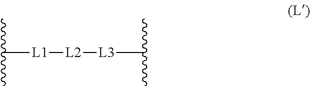

wherein:
LINK is attached to M via L1;
LINK is attached to U via L3;
L1 and L3 are each independently selected from a single bond, branched or straight-chain —(C₁₋₆ alkylene)-, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynyl, —(C₂₋₆ alkenylene)-, a divalent 3- to 6-membered cycloalkyl ring and a divalent 4- to 7-membered heterocyclyl ring;
L2 is represented by the formula -(L4)ₘ-;
each L4 is independently selected from a divalent 3- to 6-membered cycloalkyl ring, a divalent 4- to 7-membered heterocyclyl ring and a unit of formula:

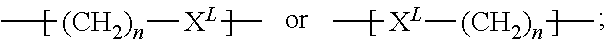

each X^{L} is independently selected from a single bond, —N(R′)—, —O—, —C(O)—, —S—, —SO—, —SO₂—, —C(R″)═C(R″)— and —C(H)—C(H)—;
n is selected from 1 to 4;
m is selected from 1 to 30;
each R′ is independently selected from H and C₁₋₄ alkyl;
each R″ is independently selected from H, fluoro and chloro; and U is:
(a) a CRBN E3 ubiquitin ligase binding moiety of formula (U1):

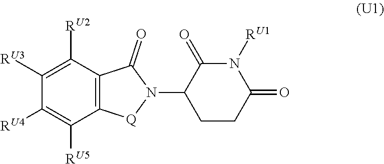

wherein:
R^{U1} is H;
Q is selected from —CH(R^{U6})—, —N(R^{U6})—, —O—, —C(O)—, —NH—CH(R^{U6})—N═C(R^{U6})—, or —N═N—;
R^{U6} is H or C₁₋₄alkyl;
R^{U2} and R^{U5} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

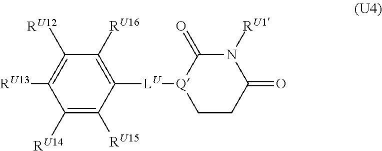

wherein:
R^{U1} is H;
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U12}, R^{U13} and R^{U14} is a single bond to LINK; R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

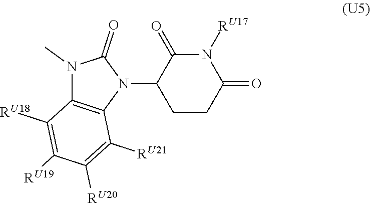

wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

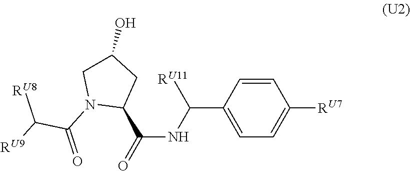

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

In one preferred aspect of these embodiments, M is of formula (IV):

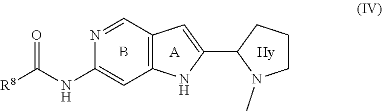

wherein R⁸ is as described herein, the group R⁸ being unsubstituted or substituted by one, two or three substituents independently selected from halo and C₁₋₄alkyl;
wherein M is bonded to LINK via a C or N atom within group R⁸ such that a hydrogen atom on the C or N atom within group R⁸ is replaced with a bond to LINK.

In this embodiment, R⁸ is preferably selected from one of the following structures:

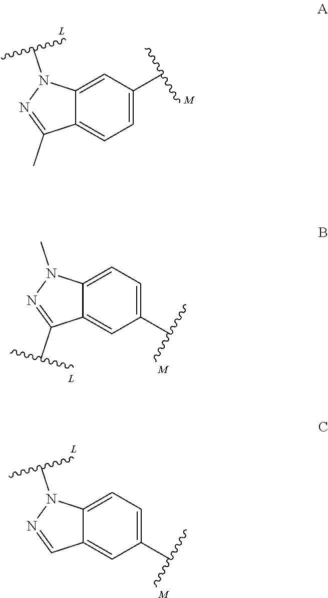

wherein:

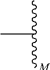

represents the point of attachment of R⁸ to the rest of moiety M;

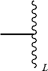

represents the point of attachment of R⁸ to LINK; and
the group R⁸ is further unsubstituted or substituted by one or two substituents independently selected from fluorine and methyl. For example, the group R⁸ may be selected from one of the following structures:

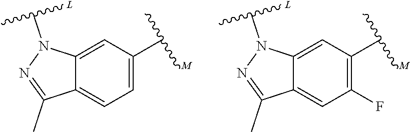

wherein:

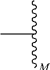

represents the point of attachment of R⁸ to the rest of moiety M; and

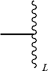

represents the point of attachment of R⁸ to LINK.

In this embodiment, L2 is preferably selected from —(CH₂)ₘ—, —(CH₂CH₂O)ₘ—, —(OCH₂CH₂)ₘ—, —(CH₂CH₂S)ₘ—, —(SCH₂CH₂)ₘ—, and a divalent 4- to 7-membered heterocyclyl ring.

In this embodiment, preferably L1 and L3 are each independently selected from a single bond, branched or straight-chain —(C₁₋₆ alkylene)-, —N(R′)—, —C(O)N(R′)—, —N(R′)C(O)—, —O—, ethynyl and a divalent 4- to 7-membered heterocyclyl ring. Preferably in this embodiment, R′ is H and the divalent 4- to 7-membered heterocyclyl ring is piperidine or diazinane. More preferably, L1 is a single bond, L2 is —(C₁₋₁₀ alkylene)- and L3 is ethynylene or a divalent moiety of piperidine (for instance ethynylene), preferably wherein L2 is —(C₃₋₈ alkylene)-, more preferably wherein L2 is —(C₇ alkylene)-.

In this embodiment, U is preferably (i) of formula (U1) wherein preferably Q is selected from —CH₂— and —C(O)—, R^{U6} is H, one of R^{U2}, R^{U3}, R^{U4} and R^{U5} is a single bond to LINK, and the rest are H, for instance wherein R^{U2} and R^{U3} are H, one of R^{U4} and R^{U5} is a single bond to LINK, and the other of R^{U4} and R^{U5} is H, or (ii) of formula (U3) wherein preferably V is S, W is N, R^{U10} is methyl, R^{U11} is H, and R^{U8} is t-butyl or phenyl, more preferably t-butyl. Most preferably, in formula (U1): Q is —CH₂—; R^{U6} is H; and R^{U2}, R^{U3} and R^{U4} are H, and R^{U5} is a single bond to LINK.

Particularly preferred compounds of the invention may be selected from:
1-(9-(2-(2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
rac-1-(8-{[2-(2,6-dioxopiperidin-3-yl)-1,3-dioxoisoindol-4-yl]amino}octyl)-3-methyl-N-[2-(1-methylpyrrolidin-2-yl)-1H-pyrrolo[3,2-c]pyridin-6-yl]indazole-6-carboxamide;
1-(8-{[2-(2,6-dioxopiperidin-3-yl)-1,3-dioxoisoindol-4-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-4-yl]amino}octyl)-3-methyl-N-{2-[(2S)-1-methylpyrrolidin-2-yl]-1H-pyrrolo[3,2-c]pyridin-6-yl}indazole-6-carboxamide;
2-(8-((2-(2,6-dioxopiperidin-3-yl)-1,3-dioxoisoindolin-4-yl)amino)octyl)-3-methyl-N-(2-((R)-1-methylpyrrolidin-2-yl)-1H-pyrrolo[3,2-c]pyridin-6-yl)-2H-indazole-6-carboxamide;
3-[4-({4-[2-(2,6-dioxopiperidin-3-yl)-6-fluoro-1,3-dioxoisoindol-5-yl]piperazin-1-yl}methyl)piperidin-1-yl]-1-methyl-N-{2-[(2R)-1-methylpyrrolidin-2-yl]-1H-pyrrolo[3,2-c]pyridin-6-yl}indazole-5-carboxamide;
1-[1-(3-{[2-(2,6-dioxopiperidin-3-yl)-1,3-dioxoisoindol-4-yl]amino}propyl)piperidin-4-yl]-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-5-yl]oxy}octyl)-3-methyl-N-{2-[(2R)-1-methylpyrrolidin-2-yl]-1H-pyrrolo[3,2-c]pyridin-6-yl}indazole-6-carboxamide;
1-{8-[3-(2,4-dioxo-1,3-diazinan-1-yl)-2-methylphenoxy]octyl}-3-methyl-N-{2-[(2R)-1-methylpyrrolidin-2-yl]-1H-pyrrolo[3,2-c]pyridin-6-yl}indazole-6-carboxamide;
1-{6-[3-(2,4-dioxo-1,3-diazinan-1-yl)-2-methylphenoxy]hexyl}-3-methyl-N-{2-[(2R)-1-methylpyrrolidin-2-yl]-1H-pyrrolo[3,2-c]pyridin-6-yl}indazole-6-carboxamide;
1-{7-[1-(2,6-dioxopiperidin-3-yl)-3-methyl-2-oxo-1,3-benzodiazol-4-yl]hept-6-yn-1-yl}-3-methyl-N-{2-[(2R)-1-methylpyrrolidin-2-yl]-1H-pyrrolo[3,2-c]pyridin-6-yl}indazole-6-carboxamide;
1-(8-((2-(2,6-dioxopiperidin-3-yl)-1,3-dioxoisoindolin-4-yl)oxy)octyl)-3-methyl-N-(2-((R)-1-methylpyrrolidin-2-yl)-1H-pyrrolo[3,2-c]pyridin-6-yl)-1H-indazole-6-carboxamide;
1-(3-((2-(2,6-dioxopiperidin-3-yl)-1,3-dioxoisoindolin-4-yl)amino)propyl)-3-methyl-N-(2-((R)-1-methylpyrrolidin-2-yl)-1H-pyrrolo[3,2-c]pyridin-6-yl)-1H-indazole-6-carboxamide;
1-(8-{[2-(2,6-dioxopiperidin-3-yl)-1,3-dioxoisoindol-5-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-5-yl]amino}octyl)-3-methyl-N-{2-[(2S)-1-methylpyrrolidin-2-yl]-1H-pyrrolo[3,2-c]pyridin-6-yl}indazole-6-carboxamide;
1-(8-{[2-(2,6-dioxopiperidin-3-yl)-1-oxo-3H-isoindol-4-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1-oxo-3H-isoindol-4-yl]amino}octyl)-3-methyl-N-{2-[(2S)-1-methylpyrrolidin-2-yl]-1H-pyrrolo[3,2-c]pyridin-6-yl}indazole-6-carboxamide;
N-(3-chloro-2-((R)-1-methylpyrrolidin-2-yl)-1H-pyrrolo[3,2-c]pyridin-6-yl)-1-(8-((2-(2,6-dioxopiperidin-3-yl)-1,3-dioxoisoindolin-4-yl)amino)octyl)-3-methyl-1H-indazole-6-carboxamide;
1-(4-{4-[2-(2,6-dioxopiperidin-3-yl)-1,3-dioxoisoindol-5-yl]piperazin-1-yl}butyl)-3-methyl-N-{2-[(2R)-1-methylpyrrolidin-2-yl]-1H-pyrrolo[3,2-c]pyridin-6-yl}indazole-6-carboxamide;
1-(4-{4-[2-(2,6-dioxopiperidin-3-yl)-1,3-dioxoisoindol-5-yl]piperazin-1-yl}butyl)-3-methyl-N-{2-[(2S)-1-methylpyrrolidin-2-yl]-1H-pyrrolo[3,2-c]pyridin-6-yl}indazole-6-carboxamide;
1-(4-((2-(2,6-dioxopiperidin-3-yl)-1,3-dioxoisoindolin-4-yl)amino)butyl)-3-methyl-N-(2-((R)-1-methylpyrrolidin-2-yl)-1H-pyrrolo[3,2-c]pyridin-6-yl)-1H-indazole-6-carboxamide;
1-(6-{[2-(2,6-dioxopiperidin-3-yl)-1,3-dioxoisoindol-4-yl]amino}hexyl)-3-methyl-N-{2-[(2R)-1-methylpyrrolidin-2-yl]-1H-pyrrolo[3,2-c]pyridin-6-yl}indazole-6-carboxamide;
1-(5-(4-(2-(2,6-dioxopiperidin-3-yl)-6-fluoro-1,3-dioxoisoindolin-5-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide;
1-(2-(2-(4-(2-(2,6-dioxopiperidin-3-yl)-6-fluoro-1,3-dioxoisoindolin-5-yl)piperazin-1-yl)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(2-(2-(2-((2-(2,6-dioxopiperidin-3-yl)-1,3-dioxoisoindolin-4-yl)amino)ethoxy)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(2-(2-((2-(2,6-dioxopiperidin-3-yl)-1,3-dioxoisoindolin-4yl)amino)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(3-(1-(2-(2,6-dioxopiperidin-3-yl)-1,3-dioxoisoindolin-4-yl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide;
1-(3-(1-(4-((2,6-dioxopiperidin-3-yl)carbamoyl)-2-fluorophenyl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide;
1-(5-(1-(2-(2,6-dioxopiperidin-3-yl)-1,3-dioxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide;
1-(7-(2-(2,6-dioxopiperidin-3-yl)-1-oxoisoindolin-4-yl) hept-6-yn-1-yl)-3-methyl-N-(2-((R)-1-methylpyrrolidin-2-yl)-1H-pyrrolo [3,2-c]pyridin-6-yl)-1H-indazole-6-carboxamide;
1-(3-((3-(2-(2,6-dioxopiperidin-3-yl)-1-oxoisoindolin-4-yl)prop-2-yn-1-yl)oxy)propyl)-3-methyl-N-(2-((R)-1-methylpyrrolidin-2-yl)-1H-pyrrolo[3,2-c]pyridin-6-yl)-1H-indazole-6-carboxamide;
1-(5-(2-(2,6-dioxopiperidin-3-yl)-1-oxoisoindolin-4-yl)pent-4-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(2-(2,6-dioxopiperidin-3-yl)-1,3-dioxoisoindolin-4-yl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(3-((2,6-dioxopiperidin-3-yl)carbamoyl)-2-fluorophenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(3-((2,6-dioxopiperidin-3-yl)carbamoyl)-2-methylphenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(8-((2-(2,6-dioxopiperidin-3-yl)-1,3-dioxoisoindolin-4-yl)amino)octyl)-N-(2-((R)-1-methylpyrrolidin-2-yl)-1H-pyrrolo[3,2-c]pyridin-6-yl)-1H-indazole-5-carboxamide;
1-(8-((2-(2,6-dioxopiperidin-3-yl)-1,3-dioxoisoindolin-4-yl)amino)octyl)-N-(2-((S)-1-methylpyrrolidin-2-yl)-1H-pyrrolo[3,2-c]pyridin-6-yl)-1H-indazole-5-carboxamide;
1-(9-(2-(2,6-dioxopiperidin-3-yl)-1-oxoisoindolin-4-yl)non-8-yn-1-yl)-N-(2-((R)-1-methylpyrrolidin-2-yl)-1H-pyrrolo[3,2-c]pyridin-6-yl)-1H-indazole-5-carboxamide;
1-(9-(1-(2,6-dioxopiperidin-3-yl)-3-methyl-2-oxo-2,3-dihydro-1H-benzo[d]imidazol-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-((E)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-((Z)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-(9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-(9-(6-(((S)-2,6-dioxopiperidin-3-yl)carbamoyl)pyridin-3-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(6-(((S)-2,6-dioxopiperidin-3-yl)carbamoyl)pyridin-2-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(((S)-2,6-dioxopiperidin-3-yl)carbamoyl)pyridin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(2-((R)-1-(methyl-d3)pyrrolidin-2-yl)-1H-pyrrolo[3,2-c]pyridin-6-yl)-1H-indazole-6-carboxamide;
1-(8-(2-(2,6-dioxopiperidin-3-yl)-1-oxoisoindolin-4-yl)oct-7-yn-1-yl)-3-methyl-N-(2-((R)-1-methylpyrrolidin-2-yl)-1H-pyrrolo[3,2-c]pyridin-6-yl)-1H-indazole-6-carboxamide;
1-(6-(2-(2,6-dioxopiperidin-3-yl)-1-oxoisoindolin-4-yl)hex-5-yn-1-yl)-3-methyl-N-(2-((R)-1-methylpyrrolidin-2-yl)-1H-pyrrolo[3,2-c]pyridin-6-yl)-1H-indazole-6-carboxamide;
1-(5-(4-(2-(2,6-dioxopiperidin-3-yl)-1,3-dioxoisoindolin-4-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-N-(2-((R)-1-ethylpyrrolidin-2-yl)-1H-pyrrolo[3,2-c]pyridin-6-yl)-3-methyl-1H-indazole-6-carboxamide;
1-(5-((3-(2-(2,6-dioxopiperidin-3-yl)-1-oxoisoindolin-4-yl)prop-2-yn-1-yl)oxy)pentyl)-3-methyl-N-(2-((R)-1-methylpyrrolidin-2-yl)-1H-pyrrolo[3,2-c]pyridin-6-yl)-1H-indazole-6-carboxamide;
1-(2-((6-(2-(2,6-dioxopiperidin-3-yl)-1-oxoisoindolin-4-yl)hex-5-yn-1-yl)oxy)ethyl)-3-methyl-N-(2-((R)-1-methylpyrrolidin-2-yl)-1H-pyrrolo[3,2-c]pyridin-6-yl)-1H-indazole-6-carboxamide;
1-(9-(3-(((S)-2,6-dioxopiperidin-3-yl)carbamoyl)-2-fluorophenyl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-7-fluoro-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
N-(3-chloro-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide;
1-(10-(2-(2,6-dioxopiperidin-3-yl)-1-oxoisoindolin-4-yl) dec-9-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrol[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(2-((R)-5-methyl-5-azaspiro[2.4]heptan-6-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(3-methyl-2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
3-cyclopropyl-1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrolo [3,2-c]pyridin-6-yl)-1H-indazole-6-carboxamide;
1-(9-(2-(2,6-dioxopiperidin-3-yl)-6-fluoro-1-oxoisoindolin-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(3-(2,6-dioxopiperidin-3-yl)-2-fluorophenyl) non-8-yn-1-yl)-3-methyl-N-(2-((R)-1-methylpyrrolidin-2-yl)-1H-pyrrolo [3,2-c]pyridin-6-yl)-1H-indazole-6-carboxamide;
1-(9-(6-(2,6-dioxopiperidin-3-yl) pyridin-2-yl) non-8-yn-1-yl)-3-methyl-N-(2-((R)-1-methylpyrrolidin-2-yl)-1H-pyrrolo [3,2-c]pyridin-6-yl)-1H-indazole-6-carboxamide;
(R)-1-(9-(3-(2,4-dioxotetrahydropyrimidin-1 (2H)-yl)-2-fluorophenyl) non-8-yn-1-yl)-3-methyl-N-(2-(1-methylpyrrolidin-2-yl)-1H-pyrrolo [3,2-c]pyridin-6-yl)-1H-indazole-6-carboxamide;
(R)-1-(9-(6-(2,4-dioxotetrahydropyrimidin-1 (2H)-yl)pyridin-2-yl)non-8-yn-1-yl)-3-methyl-N-(2-(1-methylpyrrolidin-2-yl)-1H-pyrrolo[3,2-c]pyridin-6-yl)-1H-indazole-6-carboxamide;
(R)-1-(9-(3-(2,4-dioxotetrahydropyrimidin-1 (2H)-yl)-5-fluoro-1-methyl-1H-indazol-6-yl)non-8-yn-1-yl)-3-methyl-N-(2-(1-methylpyrrolidin-2-yl)-1H-pyrrolo[3,2-c]pyridin-6-yl)-1H-indazole-6-carboxamide;
N-(3-cyano-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide;
N-(3-chloro-2-((R)-1-methylpyrrolidin-2-yl)-1H-pyrrolo[3,2-c]pyridin-6-yl)-1-(9-(2-(2,6-dioxopiperidin-3-yl)-7-fluoro-1-oxoisoindolin-4-yl)non-8-yn-1-yl)-3-methyl-1H-indazole-6-carboxamide;
1-(1-(3-(2-(2,6-dioxopiperidin-3-yl)-1-oxoisoindolin-4-yl)prop-2-yn-1-yl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-6-fluoro-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-1,3-dioxoisoindolin-4-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(3-(2-(2,6-dioxopiperidin-3-yl)-1-oxoisoindolin-4-yl)prop-2-yn-1-yl)-3-methyl-N-(2-((R)-1-methylpyrrolidin-2-yl)-1H-pyrrolo[3,2-c]pyridin-6-yl)-1H-indazole-6-carboxamide;
1-(6-(1-(6-(((S)-2,6-dioxopiperidin-3-yl)carbamoyl)pyridin-2-yl)piperidin-4-yl)hexyl)-3-methyl-N-(2-((R)-1-methylpyrrolidin-2-yl)-1H-pyrrolo[3,2-c]pyridin-6-yl)-1H-indazole-6-carboxamide;
1-(4-(5-(2-(2,6-dioxopiperidin-3-yl)-1-oxoisoindolin-4-yl) pent-4-yn-1-yl) cyclohexyl)-3-methyl-N-(2-((R)-1-methylpyrrolidin-2-yl)-1H-pyrrolo[3,2-c]pyridin-6-yl)-1H-indazole-6-carboxamide;
1-(9-(2-((rel-S)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-((rel-R)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
N-(3-chloro-2-((R)-1-methylpyrrolidin-2-yl)-1H-pyrrolo[3,2-c]pyridin-6-yl)-1-(9-(2-((rel-S)-2,6-dioxopiperidin-3-yl)-1-oxoisoindolin-4-yl)non-8-yn-1-yl)-3-methyl-1H-indazole-6-carboxamide;
N-(3-chloro-2-((R)-1-methylpyrrolidin-2-yl)-1H-pyrrolo[3,2-c]pyridin-6-yl)-1-(9-(2-((rel-R)-2,6-dioxopiperidin-3-yl)-1-oxoisoindolin-4-yl)non-8-yn-1-yl)-3-methyl-1H-indazole-6-carboxamide;
1-(9-(2-((rel-S)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
1-(9-(2-((rel-R)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
1-((1-(2-(2,6-dioxopiperidin-3-yl)-1,3-dioxoisoindolin-5-yl)piperidin-4-yl) methyl)-3-methyl-N-(2-((R)-1-methylpyrrolidin-2-yl)-1H-pyrrolo[3,2-c]pyridin-6-yl)-1H-indazole-6-carboxamide;
1-(5-(1-(2-(2,6-dioxopiperidin-3-yl)-1-oxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide;
1-((1-(2-(2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide;
1-((1-(2-((rel-S)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide;
1-((1-(2-((rel-R)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide; and
1-(5-(1-(2-(2,6-dioxopiperidin-3-yl)-1,3-dioxoisoindolin-5-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide;
   and the pharmaceutically acceptable salts thereof.

### MLLT1 and or MLLT3 Inhibitors

In a separate aspect of the present invention, compounds of formula (I′) are provided that are potent inhibitors of MLLT1 and/or MLLT3. Accordingly, the invention provides a compound which is a bicyclic compound of formula (I′) or a pharmaceutically acceptable salt thereof:

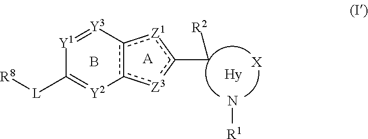

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —
C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
at least one of R⁴, R⁵ and R⁶ is not H;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
each R¹¹ is independently selected from H, C₁₋₄ alkyl, and C₁₋₄ cycloalkyl.

Typically, in formula (I′), ring Hy is a 5- to 6-membered heterocyclyl ring containing X and at least one N atom, for instance one or two N atoms. X is typically a bond, —N(R¹¹)—, O or —C(R¹¹)₂—. Preferably, X is a bond, —N(Me)-, O or —CH₂—, most preferably X is a bond. In particular, when ring Hy is a 4- or 5-membered heterocyclyl ring, X is most preferably a bond. For example, ring Hy may be selected from a pyrrolidinyl, piperidinyl, morpholinyl or diazinanyl ring. Preferably, ring Hy is a 5-membered heterocyclyl ring containing X and at least one N atom, for instance one N atom. For example, ring Hy may be a pyrrolidinyl ring.

Ring Hy is linked to ring A via a C atom within ring Hy, said C atom also being linked to R². A N atom within ring Hy is substituted by R¹. Typically, the N atom of ring Hy which is substituted by R¹ is adjacent to the C atom of ring Hy that is bonded to ring A.

Typically, in formula (I′), R¹ is H, C₁₋₄ cycloalkyl, or C₁₋₄ alkyl which is itself unsubstituted or substituted with one C₁₋₄ alkoxy or one, two or three halo, preferably with one C₁₋₄ alkoxy, for example R¹ may be H or C₁₋₂alkyl which is unsubstituted or substituted with one C₁₋₂alkoxy or one, two or three halo, preferably with one C₁₋₂ alkoxy, or R¹ may be H or C₁₋₂ alkyl which is unsubstituted or substituted with one C₁₋₂ alkoxy. Preferably, R¹ is H, methyl, ethyl or methoxyethyl, more preferably methyl or ethyl. Most preferably, R¹ is methyl.

In formula (I′), R² is H or methyl. Preferably, R² is H.

Typically, in formula (I′), each R¹¹ is independently selected from H and methyl. When X is —N(R¹¹)— or —S(O)(NR¹¹)—, R¹¹ is preferably methyl. When X is —C(R¹¹)₂—, each R¹¹ is preferably H.

Aside from groups R¹ and R², ring Hy may further be unsubstituted or substituted by one or two R³. Typically, each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring. More typically, each R³ is independently selected from methyl, ethyl, t-butyl, methoxy and fluoro, or (i) two R³ linked to adjacent C atoms in ring Hy form, together with the C atoms to which they are attached, a cyclohexyl ring, or (ii) two R³ linked to the same C atom in ring Hy form, together with the C atom to which they are attached, a cyclopropyl or cyclopentyl ring. Preferably, ring Hy is substituted by no R³ groups.

In another typical embodiment, each R³ is independently selected from methyl, ethyl, t-butyl, methoxy and fluoro, or (i) two R³ linked to adjacent C atoms in ring Hy form, together with the C atoms to which they are attached, a cyclohexyl ring, or (ii) two R³ linked to the same C atom in ring Hy form, together with the C atom to which they are attached, a cyclopropyl ring or a cyclopentyl ring. Preferably, ring Hy is substituted by two R³ groups which are both C₁₋₄ alkyl, for instance both being methyl, or one R³ group which is C₁₋₄ alkyl, for instance methyl, ethyl or t-butyl.

Typically, therefore, in formula (I′), ring Hy is a 5- to 6-membered heterocyclyl ring containing X and at least one N atom, for example a pyrrolidinyl, piperidinyl, morpholinyl or diazinanyl ring; the N atom of ring Hy which is substituted by R¹ is adjacent to the C atom of ring Hy that is bonded to ring A; R¹ is H or C₁₋₂ alkyl which is unsubstituted or substituted with one C₁₋₂ alkoxy or one, two or three halo, preferably with one C₁₋₂ alkoxy; R² is H or methyl; X is a bond, —N(R¹¹)—, O or —C(R¹¹)₂—; R¹¹ is H or methyl; ring Hy is further unsubstituted or substituted by one or two R³; and each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring.

Preferably, in formula (I′), ring Hy is a pyrrolidinyl ring; the N atom of ring Hy which is substituted by R¹ is adjacent to the C atom of ring Hy that is bonded to ring A; R¹ is methyl or ethyl, more preferably methyl; R² is H; and ring Hy is substituted by no R³ groups, i.e. ring Hy is not further substituted and carries substituent R¹ only. Alternatively, in formula (I), ring Hy is a pyrrolidinyl ring; the N atom of ring Hy which is substituted by R¹ is adjacent to the C atom of ring Hy that is bonded to ring A; R¹ is H, methyl, ethyl or methoxyethyl, more preferably methyl; R² is H; and ring Hy is further substituted by two R³ groups which are both C₁₋₄ alkyl, for instance both being methyl, or one R³ group which is C₁₋₄ alkyl, for instance methyl, ethyl or t-butyl.

Typically, in formula (I′), no more than two of Y¹, Y² and Y³ are N. Preferably, no more than one of Y¹, Y² and Y³ are N. Most preferably, Y¹ is N, Y² is —C(R⁶)—, and Y³ is —C(R⁵)—.

Typically, Z¹ is —C(R⁴)—, Z³ is —N(H)—, and Y¹ is N. Thus, the bicyclic structure formed by rings A and B has the structure:

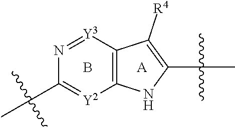

In one preferred embodiment, Z¹ is —C(R⁴)—, Z³ is —N(H)—, Y¹ is N, Y² is —C(R⁶)—, and Y³ is —C(R⁵)—, such that the bicyclic structure formed by rings A and B has the structure:

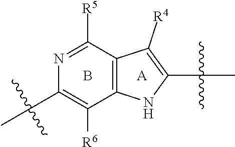

Typically, in formula (I′), one of R⁴, R⁵ and R⁶ is selected from halo, CN, C₁₋₄ alkoxy, and C₁₋₄ alkyl which is itself unsubstituted or substituted by one, two or three halo, and the rest are H, with the proviso that R⁵ and R⁶ are not CN. Preferably, one of R⁴, R⁵ and R⁶ is selected from halo (e.g. fluoro or chloro), CN, methoxy, methyl and trifluoromethyl, and the rest are H, with the proviso that R⁵ and R⁶ are not CN.

In one preferred embodiment, R⁵ and R⁶ are as defined herein and R⁴ is selected from halo, CN, C₁₋₄ alkoxy, and C₁₋₄ alkyl which is itself unsubstituted or substituted by one, two or three halo. More preferably, R⁵ and R⁶ are as defined herein and R⁴ is selected from halo, CN, C₁₋₄ alkyl and CF₃. More preferably, R⁵ and R⁶ are as defined herein and R⁴ is selected from halo, and CF₃. Most preferably, R⁵ and R⁶ are as defined herein and R⁴ is selected from F, Cl, and CF₃.

In one preferred embodiment, R⁵ is selected from H, cyclopropyl, C₁₋₄ alkoxy, and C₁₋₄ alkyl, for example from H, C₁₋₄ alkoxy, and C₁₋₄ alkyl. More preferably, R⁵ is selected from H, cyclopropyl, methoxy and methyl, for example H, methoxy and methyl. In one particularly preferred embodiment, R⁵ is selected from cyclopropyl and C₁₋₄ alkyl, for example from C₁₋₄ alkyl. More preferably, R⁵ is selected from cyclopropyl and methyl, for example methyl.

In one preferred embodiment, R⁶ is H.

Typically, therefore, in formula (I′):
R⁴ is selected from halo, CN, C₁₋₄ alkoxy, and C₁₋₄ alkyl which is itself unsubstituted or substituted by one, two or three halo, and R⁵ and R⁶ when present are H; or
R⁵ is selected from cyclopropyl, C₁₋₄ alkoxy, and C₁₋₄ alkyl, typically from C₁₋₄ alkyl, and R⁴ and R⁶ when present are H.

Preferably, in formula (I′):
R⁴ is selected from F, Cl, and CF₃, and R⁵ and R⁶ when present are H;
R⁵ is selected from cyclopropyl and methyl, typically from methyl, and R⁴ and R⁶ when present are H.

In formula (I′), L is —C(O)N(H)—, wherein the C atom of L is bonded to R⁸, and the N atom of L is bonded to ring B.

Typically, in formula (I′), R⁸ may be selected from indazolyl, thieno[2,3-c]pyrazolyl, imidazo[4,5-b]pyridinyl, pyrazolo[3,4-b]pyridinyl, furo[2,3-c]pyridinyl, indolyl, benzoxazolyl, benzothiazolyl, [1,2,4]triazolo[4,3-a]pyridinyl, thieno[2,3-d]pyrimidinyl, 1,2,3-benzotriazolyl, imidazo[1,5-a]pyridinyl, imidazo[1,2-a]pyrazinyl, oxazolo[5,4-b]pyridinyl, quinolinyl, naphthyridinyl, isoquinolinyl, quinazolinyl, and quinoxalinyl. Preferably, R⁸ is an 8- to 10-membered heteroaryl ring preferably containing one, two or three N atoms. More preferably, R⁸ is an indazolyl group.

In some preferred embodiments, R⁸ is a group selected from the following structures. For the avoidance of doubt, the following structures may be unsubstituted or substituted by one, two or three substituents as defined herein.

### [See Below]

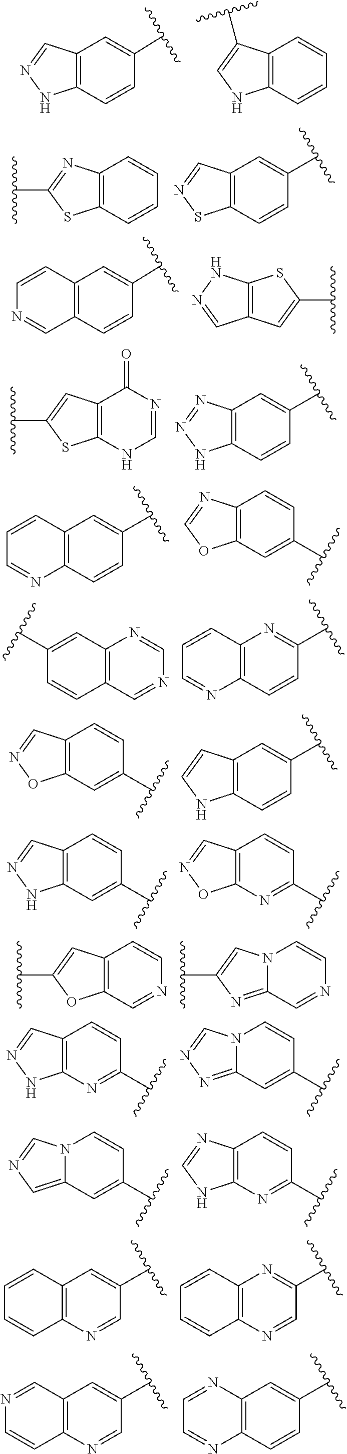

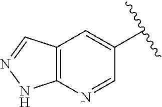 [SMILES: *c1cnc2[nH]ncc2c1]

In formula (I′), R⁸ is unsubstituted or substituted by one, two or three substituents, for instance one or two substituents. Typically, each substituent is independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂alkoxy. More typically, each substituent is independently selected from fluoro, chloro, methoxy, R¹⁰, —(CH₂)—R¹⁰, ═O, —CN, and C₁₋₂ alkyl which is itself unsubstituted or substituted by one or two groups selected from selected from halo (e.g. fluoro) and methoxy. Preferably, each substituent is independently selected from chloro, fluoro, R¹⁰ and methyl. Most preferably, each substituent is independently selected from fluoro and methyl, for instance methyl. R¹⁰ is as defined herein.

Typically, in formula (I′), R¹⁰ is a group selected from a 5- to 6-membered heteroaryl ring, a 3 to 6-membered cycloalkyl ring, a 4- to 5-membered heterocyclyl ring, and a phenyl ring. The cycloalkyl, heteroaryl and heterocyclyl rings are as defined herein. Preferably, R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably cyclopropyl and pyrazolyl.

Typically, in formula (I′), R¹⁰ is unsubstituted or substituted by one or two substituents independently selected from fluoro, methyl and methoxy. Preferably, R¹⁰ is unsubstituted.

Typically, therefore, in formula (I′), R⁸ is an 8- to 10-membered heteroaryl ring; the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy; wherein R¹⁰ is a group selected from a 5- to 6-membered heteroaryl ring, a 3- to 6-membered cycloalkyl ring, a 4- to 5-membered heterocyclyl ring, and a phenyl ring; and R¹⁰ is unsubstituted or substituted by one or two substituents independently selected from fluoro, methyl and methoxy.

Preferably, in formula (I′), R⁸ is an 8- to 10-membered heteroaryl ring containing one, two or three N atoms, for example an indazolyl group; the group R⁸ is unsubstituted or substituted by one or two substituents, each independently selected from fluoro, R¹⁰ and methyl; R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl; and R¹⁰ is unsubstituted.

In a typical embodiment of formula (I′), the bicyclic structure formed by rings A and B has the structure:

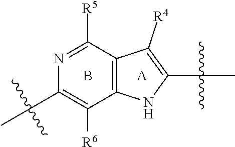

and:
ring Hy is a 5- to 6-membered heterocyclyl ring containing X and at least one N atom, for example a pyrrolidinyl, piperidinyl, morpholinyl or diazinanyl ring; wherein the N atom of ring Hy which is substituted by R¹ is adjacent to the C atom of ring Hy that is bonded to ring A;
R¹ is H or C₁₋₂ alkyl which is unsubstituted or substituted with one C₁₋₂ alkoxy or one, two or three halo, preferably with one C₁₋₂ alkoxy;
R² is H or methyl;
X is a bond, —N(Me)-, O or —CH₂—;
ring Hy is further unsubstituted or substituted by one or two R³; wherein each R³ is independently selected from C₁₋₄ alkyl, C₁₋₄ alkoxy and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring;
R⁴ is selected from CN, halo, C₁₋₄ alkoxy, and C₁₋₄ alkyl which is itself unsubstituted or substituted by one, two or three halo, and R⁵ and R⁶ when present are H;
the C atom of L is bonded to R⁸, and the N atom of L is bonded to ring B;
R⁸ is an 8- to 10-membered heteroaryl ring; wherein the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from halo and C₁₋₂ alkoxy; and
R¹⁰ is a group selected from a 5- to 6-membered heteroaryl ring, a 3- to 6-membered cycloalkyl ring, a 4- to 5-membered heterocyclyl ring, and a phenyl ring; wherein R¹⁰ is unsubstituted or substituted by one or two substituents independently selected from fluoro, methyl and methoxy.

In a preferred embodiment of formula (I′), the bicyclic structure formed by rings A and B has the structure:

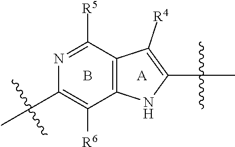

and:
ring Hy is a pyrrolidinyl ring; wherein the N atom of ring Hy which is substituted by R¹ is adjacent to the C atom of ring Hy that is bonded to ring A; R^{1′} is H, methyl, ethyl or methoxyethyl, more preferably methyl; R² is H; and ring Hy is substituted by no R³ groups; R⁴ is selected from CN, F, Cl, Me and CF₃, and R⁵ and R⁶ when present are H;
the C atom of L is bonded to R⁸, and the N atom of L is bonded to ring B;
R⁸ is an 8- to 10-membered heteroaryl ring containing one, two or three N atoms, for example an indazolyl group; the group R⁸ is unsubstituted or substituted by one or two substituents, each independently selected from fluoro, R¹⁰ and methyl; R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl; and R¹⁰ is unsubstituted.

In a particular embodiment, the compound of the invention is a bicyclic compound of formula (II′) or a pharmaceutically acceptable salt thereof:

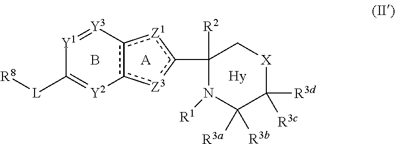

wherein:
Z¹, Z³, Y¹, Y², Y³, R¹, R², R⁸, X and L are as defined in any one of the preceding claims;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)-then neither R^{3c} nor R^{3d} are halo;
wherein at least two of R^{3a}, R^{3b}, R^{3c} and R^{3d} are H.

Typically, in formula (II′): R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is —N(R¹¹)—, O, S, —S(O)₂— or —S(O)(NR¹¹)—, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring); wherein at least two of R^{3a}, R^{3b}, R^{3c} and R^{3d} are H.

Preferably, in formula (II′), R^{3a}, R^{3b}, R^{3c} and R^{3d} are all H.

In a typical embodiment of formula (II′), the bicyclic structure formed by rings A and B has the structure:

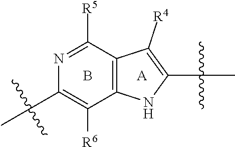

and:
R¹ is H or C₁₋₂ alkyl which is unsubstituted or substituted with one C₁₋₂ alkoxy or one, two or three halo, preferably with one C₁₋₂ alkoxy;
R² is H or methyl;
X is a bond, —N(Me)-, O or —CH₂—;
R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is NR¹¹ or O, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring);
wherein at least two of R^{3a}, R^{3b}, R^{3c} and R^{3d} are H; R⁴ is selected from CN, halo, C₁₋₄ alkoxy, and C₁₋₄ alkyl which is itself unsubstituted or substituted by one, two or three halo, and R⁵ and R⁶ when present are H;
the C atom of L is bonded to R⁸, and the N atom of L is bonded to ring B;
R⁸ is an 8- to 10-membered heteroaryl ring; wherein the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy; and
R¹⁰ is a group selected from a 5- to 6-membered heteroaryl ring, a 3- to 6-membered cycloalkyl ring, a 4- to 5-membered heterocyclyl ring, and a phenyl ring; wherein R¹⁰ is unsubstituted or substituted by one or two substituents independently selected from fluoro, methyl and methoxy.

In a preferred embodiment of formula (II′), the bicyclic structure formed by rings A and B has the structure:

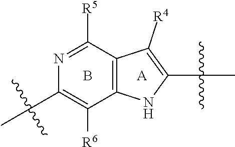

and:
X is absent;
R¹ is H, methyl, ethyl or methoxyethyl, more preferably methyl;
R² is H;
R^{3a}, R^{3b}, R^{3c} and R^{3d} are all H;
R⁴ is selected from CN, F, Cl, Me and CF₃, and R⁵ and R⁶ when present are H;
the C atom of L is bonded to R⁸, and the N atom of L is bonded to ring B; R⁸ is an 8- to 10-membered heteroaryl ring containing one, two or three N atoms, for example an indazolyl group; the group R⁸ is unsubstituted or substituted by one or two substituents, each independently selected from fluoro, R¹⁰ and methyl; R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl; and R¹⁰ is unsubstituted.

In a particularly preferred embodiment, the compound of the invention is a bicyclic compound of formula (III′) or a pharmaceutically acceptable salt thereof:

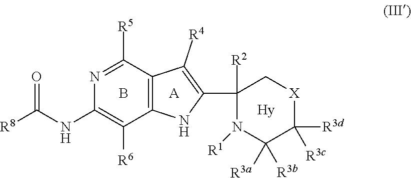

wherein R¹, R², R^{3a}, R^{3b}, R^{3c}, R^{3d}, X, R⁴, R⁵, R⁶ and R⁸ are as defined herein.

Typically, in the compounds of formula (III′):
R¹ is H or C₁₋₄ alkyl which is itself unsubstituted or substituted with C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring, with the proviso that when X is O or —N(Me)-, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from CN, halo C₁₋₄ alkoxy, and C₁₋₄ alkyl, and R⁵ and R⁶ are H;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy; and
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo.

Typically, in formula (III′), R¹ is H, or C₁₋₄ alkyl which is itself unsubstituted or substituted with one C₁₋₄ alkoxy or one, two or three halo, preferably with one C₁₋₄ alkoxy, for example R¹ may be H or C₁₋₂ alkyl which is unsubstituted or substituted with one C₁₋₂ alkoxy or one, two or three halo, preferably with one C₁₋₂ alkoxy. Preferably, R¹ is H, methyl, ethyl or methoxyethyl. Most preferably, R¹ is methyl.

In formula (III′), R² is H or methyl. Preferably, R² is H.

Typically, in formula (III′), X is a bond, —N(R¹¹)—, O or —C(R¹¹)₂—. Preferably, X is a bond, —N(Me)-, O or —CH₂—, most preferably X is a bond.

Typically, in formula (III′), each R¹¹ is independently selected from H and methyl. When X is —N(R¹¹)— or —S(O)(NR¹¹)—, R¹¹ is preferably methyl. When X is —C(R¹)₂—, each R¹¹ is preferably H.

Typically, in formula (III′), R^{3a} and R^{3b} are independently selected from H, C₁₋₄ alkyl, halo and C₁₋₄ alkoxy, in particular from H and methyl; R^{3c} and R^{3d} are independently selected from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is NR¹¹, 0, or S, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring); wherein at least two of R^{3a}, R^{3b}, R^{3c} and R^{3d} are H.

Preferably, in formula (III′), R^{3a}, R^{3b}, R^{3c} and R^{3d} are all H.

Typically, in formula (III′), R⁴ is selected from halo, CN, C₁₋₄ alkoxy, and C₁₋₄ alkyl which is itself unsubstituted or substituted by one, two or three halo. Preferably, R⁴ is selected from CN, F, Cl, Me and CF₃.

Typically, in formula (III′), R⁵ is selected from H and C₁₋₄ alkyl. Preferably, R⁵ is selected from H and methyl, more preferably H.

Typically, in formula (III′), R⁶ is H.

Typically, therefore, in formula (III′):
R⁴ is selected from halo, CN, C₁₋₄ alkoxy, and C₁₋₄ alkyl which is itself unsubstituted or substituted by one, two or three halo, and R⁵ and R⁶ are H; or
R⁵ is selected from C₁₋₄ alkyl, and R⁴ and R⁶ when present are H.

Preferably, in formula (III′):
R⁴ is selected from selected from F, Cl, Me and CF₃, and R¹ and R⁶ are H; or
R⁵ is selected from methyl, and R⁴ and R⁶ when present are H.

In one preferred embodiment of formula (III′), R⁸ is selected from indazolyl, thieno[2,3-c]pyrazolyl, imidazo[4,5-b]pyridinyl, pyrazolo[3,4-b]pyridinyl, furo[2,3-c]pyridinyl, indolyl, benzoxazolyl, benzothiazolyl, [1,2,4]triazolo[4,3-a]pyridinyl, thieno[2,3-d]pyrimidinyl, 1,2,3-benzotriazolyl, imidazo[1,5-a]pyridinyl, imidazo[1,2-a]pyrazinyl, oxazolo[5,4-b]pyridinyl, quinolinyl, naphthyridinyl, isoquinolinyl, quinazolinyl, and quinoxalinyl. Preferably, R is an 8 to 10-membered heteroaryl ring containing one, two or three N atoms. Most preferably, R⁸ is an indazolyl group.

Typically, in formula (III′), R⁸ is a group selected from the following structures. For the avoidance of doubt, the following structures may be unsubstituted or substituted by one, two or three substituents as defined herein.

### [See Below]

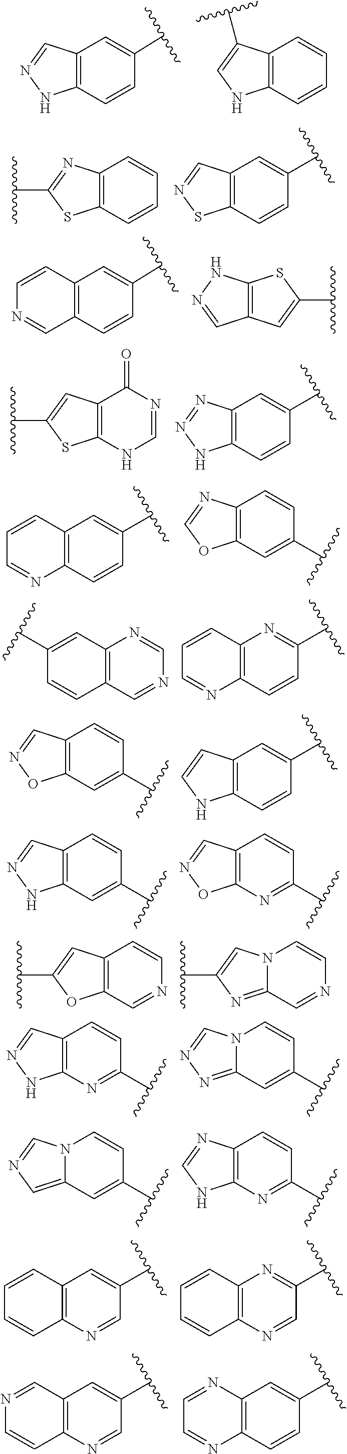

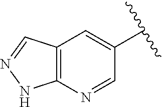 [SMILES: *c1cnc2[nH]ncc2c1]

Preferably, R⁸ is an indazolyl group.

Typically, in formula (III′), R⁸ is unsubstituted or substituted by one, two or three substituents, for instance one or two substituents, independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy. More typically, each substituent is independently selected from fluoro, chloro, methoxy, R¹⁰, —(CH₂)—R¹⁰, ═O, —CN, and C₁₋₂ alkyl which is itself unsubstituted or substituted by one or two groups selected from selected from halo (e.g. fluoro) and methoxy.

Preferably, each substituent is independently selected from fluoro, R¹⁰ and methyl. Most preferably, each substituent is independently selected from fluoro and methyl. R¹⁰ is as defined herein.

Typically, in formula (III′), R¹⁰ is a group selected from a 5- to 6-membered heteroaryl ring, a 3- to 6-membered cycloalkyl ring, a 4- to 5-membered heterocyclyl ring, and a phenyl ring. The aryl, heteroaryl and heterocyclyl rings are as defined herein. Preferably, R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl.

Typically, in in formula (III′), R¹⁰ is unsubstituted or substituted by one or two substituents independently selected from fluoro, methyl and methoxy. Preferably, R¹⁰ is unsubstituted.

In typical embodiments of formula (III′):
R¹ is H or C₁₋₂ alkyl which is unsubstituted or substituted with one C₁₋₂ alkoxy or one, two or three halo, preferably with one C₁₋₂ alkoxy;
R² is H or methyl;
X is a bond, —N(Me)-O or —C(H)₂—;
R^{3a} and R^{3b} are independently selected from H and methyl; R^{3c} and R^{3d} are independently selected from H, C₁₋₄ alkyl, halo and C₁₋₄ alkoxy, in particular from H, methyl, ethyl, fluoro, methoxy and t-butyl, or R^{3c} and R^{3d} form, together with the C atom to which they are attached, a C₅₋₆ cycloalkyl ring (in particular a cyclopentyl ring), with the proviso that when X is NR¹¹ or O, then neither R^{3c} nor R^{3d} are fluoro; or R^{3a} and R^{3c} are H, and R^{3b} and R^{3d} form, together with the C atoms to which they are attached, a C₅₋₆ cycloalkyl ring (in particular a cyclohexyl ring);
wherein at least two of R^{3a}, R^{3b}, R^{3c} and R^{3d} are H;
one of the following options applies: R⁴ is selected from halo, CN, C₁₋₄ alkoxy, and C₁₋₄ alkyl which is itself unsubstituted or substituted by one, two or three halo, and R⁵ and R⁶ are H; or R⁵ is selected from C₁₋₄ alkyl, and R⁴ and R⁶ when present are H;
the group R⁸ is unsubstituted or substituted by one, two or three substituents, for instance one or two substituents, each independently selected from halo, C₁₋₄ alkoxy, R¹⁰, —(CH₂)—R¹⁰, ═O, —CN, and C₁₋₄ alkyl which is itself unsubstituted or substituted by one or two groups selected from selected from halo and C₁₋₂ alkoxy; and
R¹⁰ is a group selected from a 5- to 6-membered heteroaryl ring, a 3- to 6-membered cycloalkyl ring, a 4- to 5-membered heterocyclyl ring, and a phenyl ring; wherein R¹⁰ is unsubstituted or substituted by one or two substituents independently selected from fluoro, methyl and methoxy; and R⁸ is a group selected from the following structures:

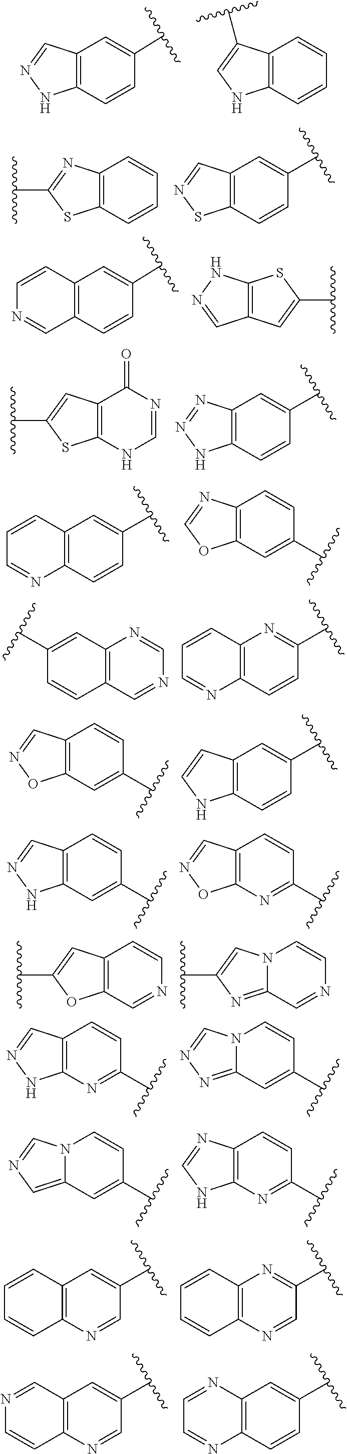

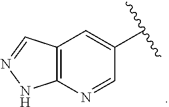 [SMILES: *c1cnc2[nH]ncc2c1]

In preferred embodiments of formula (III′),
X is absent;
R¹ is H, methyl, ethyl or methoxyethyl, more preferably methyl;
R² is H;
R^{3a}, R^{3b}, R^{3c} and R^{3d} are all H;
R⁴ is selected from F, Cl, Me and CF₃;
R⁸ is an 8- to 10-membered heteroaryl ring containing one, two or three N atoms, for example an indazolyl group; the group R⁸ is unsubstituted or substituted by one or two substituents, each independently selected from fluoro, R¹⁰ and methyl;
R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, preferably pyrazolyl; and R¹⁰ is unsubstituted.

In a most preferred embodiment, the compound of the invention is a bicyclic compound of formula (IV′) or a pharmaceutically acceptable salt thereof:

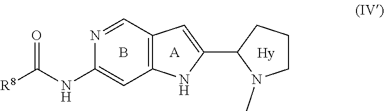

wherein R⁸ is an 8- to 10-membered heteroaryl ring, the group R⁸ being unsubstituted or substituted by one, two or three substituents independently selected from halo and C₁₋₄ alkyl.

Typically, in formula (IV′), R⁸ may be selected from thiindazolyl, thieno[2,3-c]pyrazolyl, imidazo[4,5-b]pyridinyl, pyrazolo[3,4-b]pyridinyl, furo[2,3-c]pyridinyl, indolyl, benzoxazolyl, benzothiazolyl, [1,2,4]triazolo[4,3-a]pyridinyl, thieno[2,3-d]pyrimidinyl, 1,2,3-benzotriazolyl, imidazo[1,5-a]pyridinyl, imidazo[1,2-a]pyrazinyl, oxazolo[5,4-b]pyridinyl, quinolinyl, naphthyridinyl, isoquinolinyl, quinazolinyl, and quinoxalinyl.

More typically, in formula (IV′), R⁸ is a group selected from the following structures. For the avoidance of doubt, the following structures may be unsubstituted or substituted by one, two or three substituents as defined herein.

### [See Below]

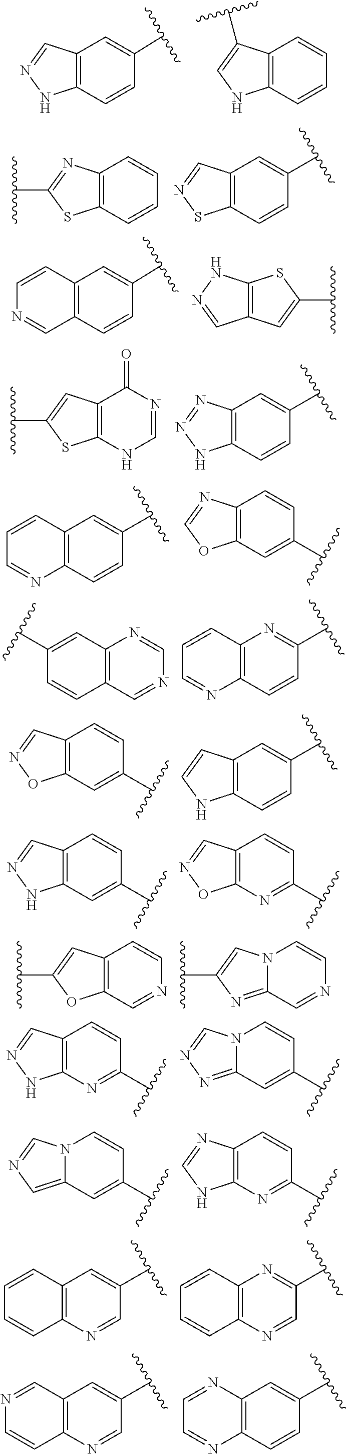

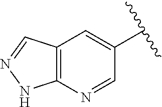 [SMILES: *c1cnc2[nH]ncc2c1]

Preferably, R⁸ is an indazolyl group.

Typically, in formula (IV′), R⁸ is unsubstituted or substituted by one, two or three substituents, for instance one or two substituents, independently selected from halo and C₁₋₄ alkyl. Preferably, each substituent is independently selected from fluoro and methyl.

In a particularly preferred embodiment, in formula (IV′), R⁸ is selected from one of the following structures:

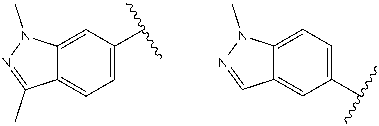 [SMILES: *c1ccc2c(C)nn(C)c2c1 ; *c1ccc2c(cnn2C)c1]

For the avoidance of doubt, R⁸ may be selected from one of the two structures above in any of formulae (I′), (II′) and (III′).

In one aspect, the following MLLT1 and/or MLLT3 inhibitors are provided:
N-{3-chloro-2-[(2R)-1-methylpyrrolidin-2-yl]-1H-pyrrolo[3,2-c]pyridin-6-yl}-1,3-dimethylindazole-6-carboxamide;
N-{3-chloro-2-[(2S)-1-methylpyrrolidin-2-yl]-1H-pyrrolo[3,2-c]pyridin-6-yl}-1,3-dimethylindazole-6-carboxamide;
(R)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide;
(S)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide;
N-{3-fluoro-2-[(rel-2S)-1-methylpyrrolidin-2-yl]-1H-pyrrolo[3,2-c]pyridin-6-yl}-1-methylindazole-5-carboxamide;
N-{3-fluoro-2-[(rel-2R)-1-methylpyrrolidin-2-yl]-1H-pyrrolo[3,2-c]pyridin-6-yl}-1-methylindazole-5-carboxamide;
N-{3-fluoro-2-[(rel-2R)-1-methylpyrrolidin-2-yl]-1H-pyrrolo[3,2-c]pyridin-6-yl}-1,3-dimethylindazole-6-carboxamide;
N-{3-fluoro-2-[(rel-2S)-1-methylpyrrolidin-2-yl]-1H-pyrrolo[3,2-c]pyridin-6-yl}-1,3-dimethylindazole-6-carboxamide;
1,3-dimethyl-N-{3-methyl-2-[(rel-2R)-1-methylpyrrolidin-2-yl]-1H-pyrrolo[3,2-c]pyridin-6-yl}indazole-6-carboxamide;
1,3-dimethyl-N-{3-methyl-2-[(rel-2S)-1-methylpyrrolidin-2-yl]-1H-pyrrolo[3,2-c]pyridin-6-yl}indazole-6-carboxamide;
1,3-dimethyl-N-{4-methyl-2-[(rel-2R)-1-methylpyrrolidin-2-yl]-1H-pyrrolo[3,2-c]pyridin-6-yl}indazole-6-carboxamide;
1,3-dimethyl-N-{4-methyl-2-[(rel-2S)-1-methylpyrrolidin-2-yl]-1H-pyrrolo[3,2-c]pyridin-6-yl}indazole-6-carboxamide; and
(R)—N-(3-cyano-2-(1-methylpyrrolidin-2-yl)-1H-pyrrolo[3,2-c]pyridin-6-yl)-1,3-dimethyl-1H-indazole-6-carboxamide;
   and the pharmaceutically acceptable salts thereof.

### Therapeutic Efficacy

The compounds of the present invention are therapeutically useful. The present invention therefore provides compounds as described herein, for use in medicine. The present invention provides compounds as described herein, for use in treating the human or animal body. For the avoidance of doubt, the agent may comprise a compound of the invention in the form of a solvate. Also provided is a pharmaceutical composition comprising a compound of the invention together with a pharmaceutically acceptable carrier or diluent. Typically, the composition contains up to 85 wt % of a compound of the invention. More typically, it contains up to 50 wt % of a compound of the invention. Preferred pharmaceutical compositions are sterile and pyrogen free. Further, when the pharmaceutical compositions provided by the invention contain a compound of the invention which is optically active, the compound of the invention is typically a substantially pure optical isomer.

The composition of the invention may be provided as a kit comprising instructions to enable the kit to be used in the methods described herein or details regarding which subjects the method may be used for.

As explained above, the compounds of the invention are useful in treating or preventing various disorders. Disorders for treatment using the compounds of the invention may include cancer. In particular, the compounds of the invention are useful for inducing selective degradation of MLLT1 and/or MLLT3, or inhibiting MLLT1 and/or MLLT3.

Cancer, e.g. acute lymphoblastic leukemia, acute myeloid leukemia, adrenocortical carcinoma, aids-related lymphoma, primary CNS lymphoma, anal cancer, astrocytomas, brain cancer, basal cell carcinoma, bile duct cancer, bladder cancer, bone cancer (e.g. ewing sarcoma, osteosarcoma and malignant fibrous histiocytoma), breast cancer, bronchial tumors, medulloblastoma and other CNS embryonal tumors, cervical cancer, chronic lymphocytic leukemia, chronic myelogenous leukemia, chronic myeloproliferative neoplasms, colorectal cancer, craniopharyngioma, endometrial cancer, ependymoma, esophageal cancer, esthesioneuroblastoma, ewing sarcoma, extragonadal germ cell tumor, intraocular melanoma, retinoblastoma, fallopian tube cancer, gallbladder cancer, gastric cancer, gastrointestinal carcinoid tumor, gastrointestinal stromal tumors (gist), germ cell tumors, extragonadal germ cell tumors, ovarian germ cell tumors, testicular cancer, gestational trophoblastic disease, hairy cell leukemia, hepatocellular cancer, histiocytosis, langerhans cell, hodgkin lymphoma, hypopharyngeal cancer, intraocular melanoma, islet cell tumors, pancreatic neuroendocrine tumors, kaposi sarcoma, kidney (renal cell) cancer, langerhans cell histiocytosis, laryngeal cancer, leukemia, liver cancer, lung cancer (non-small cell, small cell, pleuropulmonary blastoma, and tracheobronchial tumor), lymphoma, malignant fibrous histiocytoma of bone and osteosarcoma, merkel cell carcinoma, mesothelioma, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, neuroblastoma, non-hodgkin lymphoma, oropharyngeal cancer, osteosarcoma and undifferentiated pleomorphic sarcoma, pancreatic cancer, pancreatic neuroendocrine tumors (islet cell tumors), papillomatosis, paraganglioma, parathyroid cancer, penile cancer, pharyngeal cancer, pheochromocytoma, pituitary tumor, prostate cancer, rectal cancer, retinoblastoma, rhabdomyosarcoma, t-cell lymphoma, testicular cancer, throat cancer, thymoma and thymic carcinoma, thyroid cancer, tracheobronchial tumors and the like is particularly suitable to being treated with the compounds of the invention provided herein.

Typically, the compounds of the invention are for use in treating cancers which are transcriptionally addicted cancers, such as breast cancer, acute leukemia, chronic leukemia, prostate cancer, bladder cancer, cholangiocarcinoma, colon adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, pheochromocytoma and paraganglioma, rectum adenocarcinoma, stomach adenocarcinoma, uterine corpus endometrial carcinoma, cervical squamous cell carcinoma and endocervical adenocarcinoma, lung squamous cell carcinoma and sarcoma (Cell Rep, 2021, 16, 1087).

Preferably, the compounds of the invention are for use in treating acute myeloid leukaemia (AML) or acute lymphoblastic leukaemia (ALL), such as mixed-lineage leukaemia (MLL) rearranged acute leukemia (AML and ALL), NPM1 mutant acute leukemia, RUNX1-fusion acute leukemia, E2A-fusion acute leukemia, PML-fusion acute leukemia or NUP98-fusion acute leukemia.

The compounds of the invention may be used as standalone therapeutic agents. Alternatively, they may be used in combination with other active agents such as chemotherapeutic agents, targeted precision medicines or immune oncology agents. For example, they may be used in combination with a menin inhibitor (for example revumenib, ziftomenib, bleximenib), Bcl2 targeted drug (for instance venetoclax), FLT3 inhibitor (for instance sorafenib, quizartinib or gilteritinib), IDH inhibitor (for instance enasidenib), EGFR inhibitor (for instance erlotinib, gefitinib, lapatinib or osimertinib), CDK4/6 inhibitor (for instance abemaciclib, palbociclib or ribociclib), a chemotherapy (for instance cytarabine, daunorubicin, azacitidine, cedazuridine, all trans retinoic acid, doxorubicin, gemcitabine, cisplatin or paclitaxel) or an immune checkpoint inhibitor (for instance pembrolizumab, nivolumab, durvalumab or cemiplimab).

When used to treat a cancer, the compounds of the invention may be used in alleviating, ameliorating or preventing aggravation of the symptoms of the cancer. Typically, treating a cancer may comprise reducing progression of the cancer, e.g. increasing progression free survival (PFS) and/or increasing survival e.g. increasing overall survival (OS). Treating a cancer may comprise preventing or inhibiting growth of a tumour associated with the cancer. Treating a cancer may comprise preventing metastasis of the cancer. Preferably, treating a cancer may comprise reducing the size of a tumour associated with the cancer. As such, the treatment may cause tumour regression in the cancer. Treating a cancer may comprise reducing the number of tumours or lesions present in the patient. When the treatment reduces the size of a tumour associated with the cancer, the size of the tumour is typically reduced from base line by at least 10%. Base line is the size of the tumour at the date treatment with the compound is first started. The size of the tumour is typically as measured in accordance with version 1.1 of the RECIST criteria (for instance as described in Eisenhauer et al, European Journal of Cancer 45 (2009) 228-247).

The response to the treatment with the compound may be complete response, partial response or stable disease, in accordance with version 1.1 of the RECIST criteria. Preferably, the response is partial response or complete response. The treatment may achieve progression free survival for at least 60 days, at least 120 days or at least 180 days.

The reduction in tumour size may be greater 20%, greater than 30% or greater than 50% reduction relative to base line. The reduction in tumour size may be observed after 30 days of treatment or after 60 days of treatment.

In haematological cancers, treating a cancer preferentially may lead to complete remission (CR) of the cancer or it may lead to complete remission with incomplete haematological recovery (CRh) or complete remission but with incomplete platelet recovery (CRp). Additionally, treatment may lead to an increase in duration of remission (DoR), an increase in minimal residual disease (MRD), or an increase in relapse free survival (RFS) (Blood 2007 109 1815 and Leukemia Res 2018, 68, 32).

As explained here, the compounds of the invention are useful in treating or preventing various disorders. The present invention therefore provides a compound of the invention for use in medicine. The invention also provides the use of a compound of the invention in the manufacture of a medicament. The invention also provides compositions and products comprising the compounds of the invention. Such compositions and products are also useful in treating or preventing disorders. The present invention therefore provides a composition or product as defined herein for use in medicine. The invention also provides the use of a composition or product of the invention in the manufacture of a medicament. Also provided is a method of treating a subject in need of such treatment, said method comprising administering to the subject a compound of the invention. In some embodiments the subject suffers from or is at risk of suffering from one of the disorders disclosed herein.

In one aspect, the subject is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject can be any animal that is capable of being infected by a bacterium.

A subject is typically a human patient. The patient may be male or female. The age of the patient is typically at least 18 years, for instance from 30 to 70 years or from 40 to 60 years. Alternatively, the patient may be a child, for instance the patient may be under 18 years of age, or under 12 years of age. In one embodiment, the patient is a child under ten years of age or an infant under two years of age. In one embodiment the invention provides a compound as defined herein for use in treating cancer in paediatric patients.

A compound or composition of the invention can be administered to the subject in order to treat one or more symptoms of the disorder. In this embodiment, the subject is typically symptomatic. A therapeutically effective amount of the agent or formulation is administered to such a subject. A therapeutically effective amount is an amount effective to ameliorate one or more symptoms of the disorder.

The compound or composition of the invention may be administered in a variety of dosage forms. Thus, it can be administered orally, for example as tablets, troches, lozenges, aqueous or oily suspensions, dispersible powders or granules. The compound or composition of the invention may also be administered parenterally, whether subcutaneously, intravenously, intramuscularly, intrasternally, transdermally or by infusion techniques. The compound or composition may also be administered as a suppository. Preferably, the compound, composition or combination may be administered orally.

The compound or composition of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

A therapeutically effective amount of the compound or composition of the invention is administered to a subject. The dose may be determined according to various parameters, especially according to the compound used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific compound, the age, weight and conditions of the subject to be treated, the type and severity of the disease and the frequency and route of administration. Preferably, daily dosage levels are from 5 mg to 2 g.

When the compound or composition of the invention is administered to a subject in combination with another active agent, the dose of the other active agent can be determined as described above. The dose may be determined according to various parameters, especially according to the agent used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific agent, the age, weight and conditions of the subject to be treated, the type and severity of the disease and the frequency and route of administration. Preferably, daily dosage levels are from 5 mg to 2 g.

### Synthesis

Compounds of the invention can be prepared by the synthetic methods described in the Examples that follow, or by analogy with such methods using appropriate starting materials and methodologies familiar to the skilled chemist.

The following examples illustrate the invention. They do not however limit the invention in any way. In this regard, it is important to understand that the particular assay used in the Examples section is designed only to provide an indication of biological activity. There are many assays available to determine biological activity, and a negative result in any one particular assay is therefore not determinative. The invention is defined according to the claims.

### EXAMPLES

In the examples which follow below, compounds may be produced in racemic form. Compounds in racemic form are indicated by use of the term rac-in advance of the IUPAC name. In some instances, as indicated, enantiomers and/or diastereomers are separated. In general, the enantiomers and/or diastereomers of the invention can be separated using chiral chromatography. The separated enantiomers and/or diastereomers may be identified in the order in which they elute, for example the first, second, third or fourth eluting isomer respectively, as obtained by separation using chiral chromatography. The isomers may alternatively or additionally be identified using IUPAC naming conventions. Thus, where enantiomers and/or diastereomers have been assigned an absolute stereochemistry, this is indicated as R or S stereochemistry, as appropriate, and by use of a wedge-shaped bond (e.g. [symbol] ) in the chemical formula.

Where enantiomers have been separated but have undefined absolute stereochemistry the term “rel-R” or “rel-S” has been used. In these examples it is understood that the absolute configuration is unknown and the “rel-R” and “rel-S” labels are used merely to distinguish the two enantiomers from one another. The absolute stereochemistry of an enantiomer labelled as “rel-R” may be either R or S. In either case, the corresponding “rel-S” enantiomer has the opposite configuration to the “rel-R” enantiomer. In these examples the use of a rectangular bond (e.g. [symbol] ) has been used to define the “rel” configuration. This labelling convention also applies where diastereomers have been separated and the absolute configuration of a first chiral centre is known but the absolute configuration of a second chiral centre is unknown. In these examples, the known absolute configuration of the first chiral centre is indicated as R or S stereochemistry, as appropriate, and by use of a wedge-shaped bond (e.g. [symbol] ) in the chemical formula, while the unknown absolute configuration of the second chiral centre is indicated as “rel-R” or “rel-S”, and by use of a rectangular bond (e.g. [symbol] ) in the chemical formula.

Alternatively, where diastereomers have been separated and the absolute configuration of both chiral centres is unknown they may be assigned a relative stereochemistry, which denotes the stereochemistry of a first chiral centre with respect to a second chiral centre. Where a relative stereochemistry has been assigned, this is indicated by the term “rel” in the IUPAC name, and by use of a rectangular-shaped bond (e.g. [symbol] ). Where diastereomers have been separated and the relative and absolute configuration is unknown, the compounds have been labelled by the order in which they elute, for example the first, second, third or fourth eluting isomer respectively, as obtained by separation using chiral chromatography.

The moieties U of formulae (U1), (U4) when Q′ is CH and (U5) typically contain at least one chiral centre at the carbon atoms marked with an asterisk as depicted below:

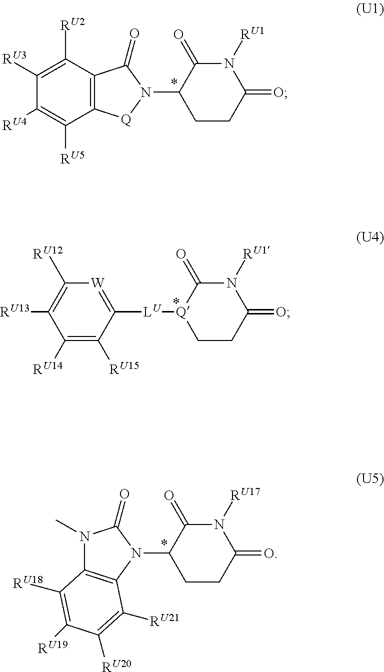

Specifically in relation to the examples that follow, unless otherwise stated, it is understood that the moiety U is typically in the form of a 1:1 mixture of the R- and S-enantiomers at said carbon atom, indicated specifically in the examples that follow by the use of a simple line-shaped bond (e.g. [symbol] ). In certain examples, the enantiomers or diastereomers have been separated such that the compounds are in enantiomerically or diastereomerically pure form. In these examples, when the stereochemistry at the chiral carbon atom in moiety U is known, the known absolute configuration is indicated as R or S stereochemistry, as appropriate, and by use of a wedge-shaped bond (e.g. [symbol] ). When the stereochemistry at the chiral carbon atom in moiety U is unknown, the unknown absolute configuration is indicated as “rel-R” or “rel-S”, and by use of a rectangular bond (e.g. [symbol] ).

Temperatures are given in degrees Celsius (° C.). The reactants used in the examples below may be obtained from commercial sources or they may be prepared from commercially available starting materials as described herein or by methods known in the art. All the compounds of the invention are synthesized according to the Examples described herein. The progress of the reactions described herein were followed as appropriate by e.g. LC, GC or TLC, and as the skilled person will readily realize, reaction times and temperatures may be adjusted accordingly.

### NMR Spectroscopy:

Solids/oils were solubilized in DMSO-d₆ or MeOH-d₄, vortexed vigorously until the solution was clear and transferred to an NMR tube for data acquisition.

Liquid-state NMR experiments were recorded using:
600 MHz (14.1 Tesla) Bruker Avance III NMR spectrometer (600 MHz for ¹H, 151 MHz for ¹³C) using a triple-resonance ¹H, ¹⁵N, ¹³C CP-TCI 5 mm cryoprobe (Bruker Biospin, Germany)
500 MHz (11.75 Tesla) Bruker Avance I NMR spectrometer (500 MHz for ¹H, 125 MHz for ¹³C) using a Dual Resonance BBI 5 mm probe (Bruker Biospin, Germany)
400 MHz (9.4 Tesla) Bruker Avance NEO NMR spectrometer (400 MHz for ¹H, 100 MHz for ¹³C) using a SEI 5 mm probe (Bruker Biospin, Germany)
400 MHz (9.4 Tesla) Bruker Avance NEO NMR spectrometer using a PI HR-BBO400S1-BBF/H/D-5.0-Z SP probe (Bruker Biospin, Germany)
300 MHz Bruker AVANCE III HD NMR spectrometer using a PA BBO 300S1 BBF—H-D-05 Z probe (Bruker Biospin, Germany)
300 MHz Bruker Avance NEO NMR spectrometer using a PA BBO BBF—H-D-05 Z (Bruker Biospin, Germany)

All the experiments used for the resonance assignment procedure and the elucidation of the products structure (1D ¹H, 2D ¹H-¹H-COSY, 2D ¹H-¹H-ROESY, 2D ¹H-¹³C-HSQC, 2D ¹H-¹³C-HMBC) were recorded at 300 K. ¹H chemical shifts are reported in δ ppm as s (singlet), d (doublet), t (triplet), q (quartet), dd (double doublet), m (multiplet) or br s (broad singlet).

### UPLC-MS Chromatography:

UPLC-MS chromatography analysis were recorded using the following apparatus using:
Waters UPLC: Acquity, UV: Acquity PDA, MS: Qda, ELSD
Waters UPLC: Acquity, UV: Acquity TUV, MS: Qda
Waters UPLC: Acquity, UV: Acquity PDA, MS: Qda

The apparatus was tested using a CSH C18 Waters column (50×2.1 mm), 1.7 μm. It used a combination of the following eluents: H₂O+0.05% TFA (v/v) (solvent A) and MeCN+0.035% TFA (v/v) (solvent B) and a positive electrospray ES+ as ionization mode. The UV detection was set at 220 and 254 nm. The methods used were the following:
Polar method (1.7 min run): gradient t=0.2% B, t=0.5 min 2% B, t=1.5 min 98% B, t=1.52 min 2% B, t=1.7 min 2% B
Polar method (3.5 min run): gradient t=0.2% B, t=1.0 min 2% B, t=2.4 min 98% B, t=3.00 min 98% B, t=3.03 min 2% B, t=3.5 min 2% B
Normal method (1.7 min run): gradient t=0.2% B, t=1.0 min 98% B, t=1.5 min 98% B, t=1.52 min 2% B, t=1.7 min 2% B
Normal method (3.5 min run): gradient t=0.2% B, t=2.4 min 98% B, t=3.0 min 98% B, t=3.03 min 2% B, t=3.5 min 2% B

Where it is observed mass spectra analysis is reported as [M+H]⁺ for the molecular ion; Someone skilled in the art will also appreciate that isotope patterns may be reported where evident, for example [M+H+2]⁺ represents the higher mass isotopic peak of Br if it is present in the molecule; Additionally, some fragmentation ions may be included in the analysis, for example [M+H-tBu]⁺, [M+H-100]⁺, [M+H-Boc]⁺ and [M-Cl+MeOH]⁺.

### Preparative HPLC

Where Example purification has been performed by preparative HPLC (chiral or achiral) the conditions are included in the reaction Step where the purification was performed or one of the general Methods below was used:

### Method A:

Gilson system used for preparative HPLC purification purposes. The system is equipped with a 333 pump A and a 334 pump B, Gilson Verity 1741 detector and GX-271 liquid handler fraction collector. The reverse phase purification was carried out by using a preparative column X-BRIDGE C18 (250×30 mm), 5 μM. Gradient elution was done using 10 mmol ammonium bicarbonate in water (as Phase A) and 100% Acetonitrile (as Phase B) with a gradient Program (B %): 10% B at 0 min hold until 3 min, 35% B at 10.0 min, 65% B at 35 min, 99% B at 35.1 min hold till 40.0 min, 10% B at 40.1 min hold until 45 min. Flow rate: 25 mL/min.

### Method B:

Gilson system used for preparative HPLC purification purposes. The system is equipped with a 333 pump A and a 334 pump B, Gilson Verity 1741 detector and GX-271 liquid handler Fraction collector. The reverse phase purification is carried out by using preparative column YMC-ACTUS C18 (250*20 mm) 5 μM. Gradient elution is done with 0.1% Formic acid in water (as Phase A) and 100% Acetonitrile (Phase B) with a gradient Program (B %): 5% B at 0 min hold till 5 min, 15% B at 200.0 min, 35% B at 35 min, 99% B at 35.1 min hold till 40.0 min, 5% B at 40.1 min hold till 45 min. Flow rate: 18 mL/min.

### Abbreviations

In addition to the definitions above, the following abbreviations are used in the Example experimental procedures below. If an abbreviation used herein is not defined, it has its generally accepted meaning:
Ac Acetyl
AcOH Acetic acid
AIBN 2,2′-Azobis(2-methylpropionitrile)
Boc tert-butyloxycarbonyl
Boc₂O Di-tertbutyl decarbonate
BrettPhos 2-(Dicyclohexylphosphino)3,6-dimethoxy-2′,4′,6′-triisopropyl-1,1′-biphenyl
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
CBr₄ tetrabromomethane
Cs₂CO₃ Cesium carbonate
CuI Copper (I) iodide
DBU 1,8-Diazabicyclo[5.4.0]undec-7-ene
DCM Dichloromethane
DHP 3,4-Dihydro-2H-pyran
DIAD Diisopropyl azodicarboxylate
DIPEA N,N-Diisopropylethylamine
Dioxane 1,4-dioxane
DMAP 4-Dimethylaminopyridine
DMEDA N,N′-dimethylethylenediamine
DMF Dimethylformamide
DMSO Dimethylsulfoxide
DMSO-d₆ Hexadeuterodimethylsulfoxide
ee Enantiomeric excess
Et Ethyl
EtOAc Ethyl acetate
Et₃N Triethylamine
EtOH Ethanol
Et₂O Diethylether
EPhos Pd G4 Methanesulfonato{Dicyclohexyl[3-(1-methylethoxy)-2′,4′,6′-tris(1-methylethyl)[1,1′-biphenyl]-2-yl]phosphine}(2′-methylamino-1,1′-biphenyl-2-yl)palladium(II)
FeCl₃ Iron(III) chloride
GC Gas chromatography
GPhos (3-(tert-Butoxy)-2′,6′-diisopropyl-6-methoxy-[1,1′-biphenyl]-2-yl)dicyclohexylphosphane
Gphos Pd G₆ TES GPhos OAc precatalyst TES
h hour
H₂O water
HATU 1-[Bis(dimethylamino)methylene]-1H-1,2,3-triazolo[4,5-b]pyridinium 3-oxid hexafluorophosphate
HCl Hydrochloric acid
Het Heteroaromatic
HPLC High performance liquid chromatography
IPA Isopropyl amine
K₂CO₃ Potassium carbonate
KI Potassium iodide
LC Liquid chromatography
LDA Lithium diisopropylamide
LiBH₄ Lithium borohydride
LiHMDS Lithium bis(trimethylsilyl)amide
LiOH Lithium hydroxide
min Minutes
Me Methyl
MeCN Acetonitrile
MeOH Methanol
MeTHF 2-Methyltetrahydrofuran
MgSO₄ Magnesium sulfate
MS Mass spectrometry
MsCl Methanesulfonyl chloride
MsOH Methanesulfonic acid
MTBE Methyl tert-butyl ether
MW Microwave
N₂ Nitrogen
NaH Sodium hydride
NaOCN Sodium cyanate
NaOH Sodium hydroxide
Na₂SO₄ Sodium sulfate
NaBH₃CN Sodium cyanoborohydride
NBS N-bromosuccinimide
NCS N-chlorosuccinimide
NH₄Cl Ammonium chloride
NIS N-iodosuccinimide
NMP N-methyl-2-pyrrolidone
Pd/C palladium on carbon
Pd(dppf)Cl₂ [1,1′-Bis(diphenylphosphino)ferrocene]dichloropalladium(II)
Pd(PPh₃)₂Cl₂ Bis(triphenylphosphine)palladium(II) dichloride
PE Petroleum ether
PPh₃ Triphenylphosphine
PMB-Cl 4-methoxybenzyl chloride
rt room temperature (18 to 22° C.)
Selectfluor 1-(Chloromethyl)-4-fluoro-1,4-diazabicyclo[2.2.2]octane-1,4-diium ditetrafluoroborate
SEM-Cl 2-(Trimethylsilyl)ethoxymethyl chloride
TBS-Cl tert-Butyldimethylsilyl chloride
tBuOK Potassium tert-butoxide
TFA Trifluoroacetic acid
TfOH Trifluoromethanesulfonic acid
TLC Thin layer chromatography
THF Tetrahydrofuran
TsCl p-Toluenesulfonyl chloride
TsOH p-Toluenesulfonic acid monohydrate
UPLC Ultra Performance Liquid Chromatography
Xantphos 4,5-Bis(diphenylphosphino)-9,9-dimethylxanthene

### Example 1 was Synthesised Following Scheme 1

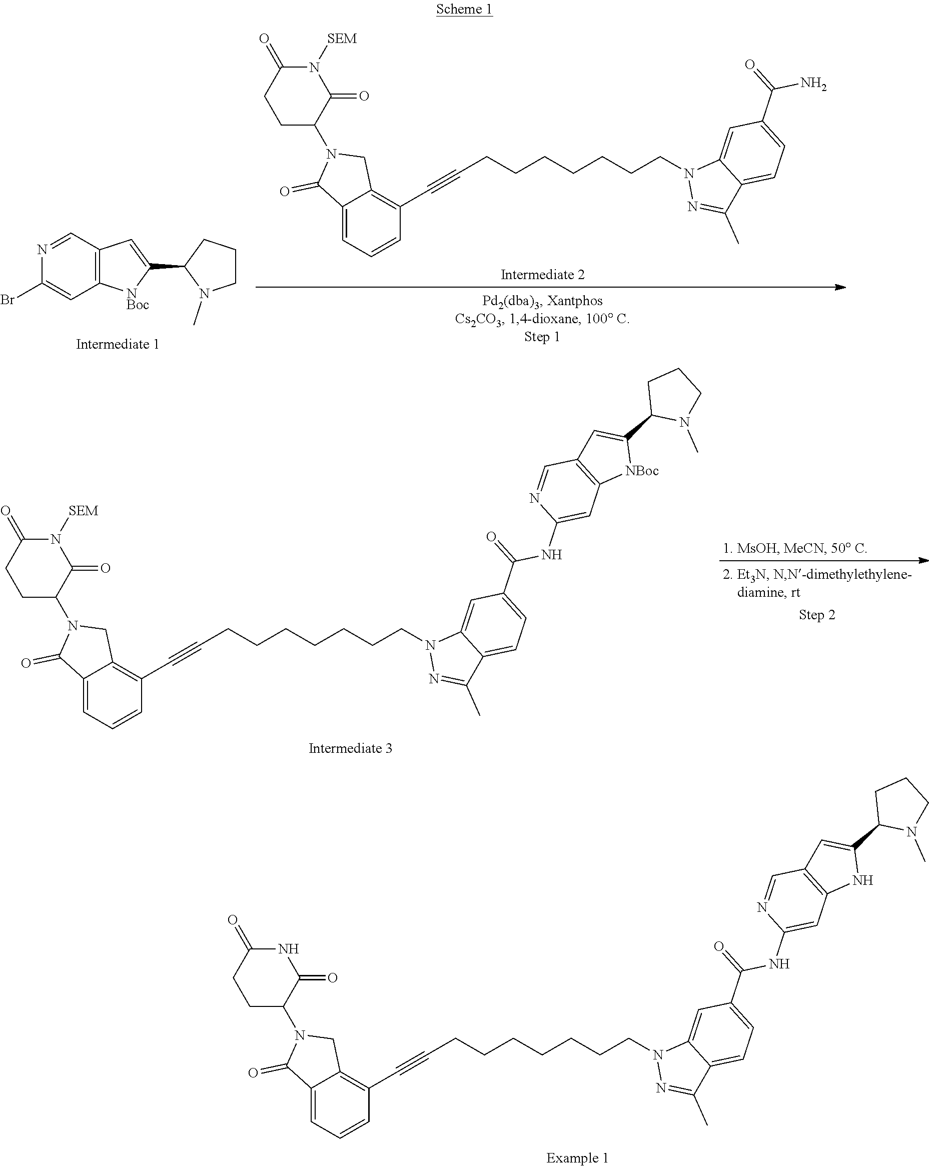 [SMILES: CN1CCC[C@@H]1c1cc2cnc(Br)cc2n1C(=O)OC(C)(C)C ; Cc1nn(CCCCCCCC#Cc2cccc3c2CN(C2CCC(=O)N(COCC[Si](C)(C)C)C2=O)C3=O)c2cc(C(N)=O)ccc12 ; Cc1nn(CCCCCCCC#Cc2cccc3c2CN(C2CCC(=O)N(COCC[Si](C)(C)C)C2=O)C3=O)c2cc(C(=O)Nc3cc4c(cn3)cc([C@H]3CCCN3C)n4C(=O)OC(C)(C)C)ccc12 ; Cc1nn(CCCCCCCC#Cc2cccc3c2CN(C2CCC(=O)NC2=O)C3=O)c2cc(C(=O)Nc3cc4[nH]c([C@H]5CCCN5C)cc4cn3)ccc12]

### Step 1

### Intermediate 3: tert-butyl-6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 2, 0.8 g, 1 mmol) in 1,4-dioxane (20 mL) was added tert-butyl-(R)-6-bromo-2-(1-methylpyrrolidin-2-yl)-1H-pyrrolo[3,2-c]pyridine-1-carboxylate (Intermediate 1, 1.00 g, 2.63 mmol) followed by cesium carbonate (2.5 g, 7.7 mmol). The reaction mixture was purged with argon for 15 min, then Pd₂(dba)₃ (0.15 g, 0.16 mmol) and Xantphos (0.3 g, 0.5 mmol) were added. The reaction mixture was further purged with argon for 10 min and then heated at 100° C. for 2 h. The reaction mixture was filtered through celite and washed with ethyl acetate (50 mL). The filtrate was concentrated in vacuo and the resultant crude material was purified by combi-flash column chromatography, by eluting with 100% ethyl acetate in heptane, to afford tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((R)-1-methylpyrrolidin-2-yl)-1H-pyrrolo [3,2-c]pyridine-1-carboxylate (Intermediate 3, 1.00 g, 39%) as a yellow solid. ¹H NMR (400 MHz, DMSO-d₆) δ ppm −0.05 (s, 9H), 0.76-0.84 (m, 2H), 1.25-1.45 (m, 7H), 1.59-1.66 (m, 2H), 1.70 (s, 9H), 1.73-1.80 (m, 2H), 1.82-1.92 (m, 3H), 2.04 (s, 3H), 2.31-2.34 (m, 1H), 2.36 (s, 3H), 2.42-2.47 (m, 3H), 2.72-2.81 (m, 2H), 2.98-3.17 (m, 3H), 3.46-3.55 (m, 1H), 3.88-3.96 (m, 1H), 4.21-4.26 (m, 1H), 4.35-4.47 (m, 3H), 4.99-5.10 (m, 2H), 5.24-5.28 (m, 1H), 6.76 (s, 1H), 7.47-7.52 (m, 1H), 7.58-7.62 (m, 1H), 7.68-7.73 (m, 2H), 7.76-7.81 (m, 1H), 8.42 (s, 1H), 8.60 (s, 1H), 8.97 (s, 1H), 10.74 (s, 1H). Mass spec: m/z: 969.2 [M+H]⁺.

### Step 2

### Example 1: 1-(9-(2-(2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl-6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 3, 5.00 g, 5.16 mmol) in acetonitrile (50 mL) was added methanesulfonic acid (3.35 mL, 51.59 mmol) and the reaction mixture was then heated at 50° C. for 2 h. After cooling to room temperature, N,N′-dimethylethylenediamine (2.78 mL, 25.79 mmol) followed by triethylamine (14.4 mL, 103.2 mmol) were added and the reaction was stirred at room temperature for 3 h. The reaction mixture was quenched with water (20 mL), the resultant precipitate was collected by filtration and dried in vacuo to afford the crude compound. The crude material was purified by preparative HPLC (Method A) to afford 1-(9-(2-(2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 1, 2.00 g, 52%) as an off-white solid. ¹H NMR (400 MHz, DMSO-d₆) δ ppm 1.22-1.46 (m, 7H), 1.50-1.59 (m, 2H), 1.76-1.94 (m, 5H), 1.96-2.03 (m, 1H), 2.12-2.15 (m, 1H), 2.17 (s, 3H), 2.24-2.30 (m, 1H), 2.41-2.44 (m, 1H), 2.45 (s, 3H), 2.53-2.62 (m, 2H), 2.85-2.95 (m, 1H), 3.13-3.17 (m, 1H), 3.34-3.36 (m, 1H), 4.24-4.33 (m, 1H), 4.37-4.47 (m, 3H), 5.10-5.15 (m, 1H), 6.40 (s, 1H), 7.45-7.52 (m, 1H), 7.58-7.60 (m, 1H), 7.67-7.80 (m, 3H), 8.25 (s, 1H), 8.43 (s, 1H), 8.52 (s, 1H), 10.52 (s, 1H), 11.00 (s, 1H), 11.38 (s, 1H). Mass spec: m/z: 739.3 [M+H]⁺.

### Intermediate 1 was Synthesised Following Scheme 2

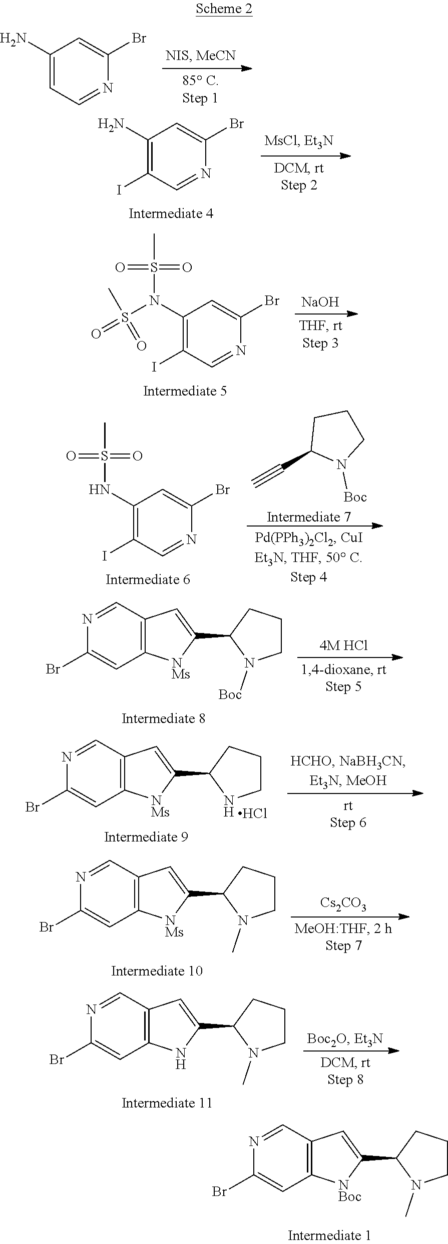

### Step 1

### Intermediate 4: 2-bromo-5-iodopyridin-4-amine

To solution of 2-bromopyridin-4-amine (CAS No: 7598-35-8, 200 g, 1156.0 mmol) in acetonitrile (1 L) was added N-iodosuccinimide (297.23 g, 1294.7 mmol) and the reaction mixture was heated at 80° C. for 16 h. The reaction mixture was then cooled to room temperature and concentrated in vacuo. The obtained residue was diluted with aqueous saturated sodium thiosulphate solution (2 L) and extracted with ethyl acetate (3×2 L). The combined organic layers were dried over anhydrous Na₂SO₄ and concentrated in vacuo to obtain the crude compound. The crude material was purified by combi-flash chromatography, by eluting with 5% ethyl acetate in heptane, to afford 2-bromo-5-iodo-pyridin-4-amine (Intermediate 4, 115 g, 33%) as a yellow solid. ¹H NMR (400 MHz, DMSO-d₆) δ ppm 6.48 (br s, 2H), 6.77 (s, 1H), 8.16 (s, 1H). Mass spec: m/z: 300.8 [M+H]⁺.

### Step 2

### Intermediate 5: N-(2-bromo-5-iodopyridin-4-yl)-N-(methylsulfonyl)methanesulfonamide

To a cooled (0° C.) solution of 2-bromo-5-iodo-pyridin-4-amine (Intermediate 4, 100 g, 334.55 mmol) in dichloromethane (2.50 g, 29 mmol) was added triethylamine (234 mL, 1673 mmol) followed by methanesulfonyl chloride (106 mL, 1338.2 mmol) in dichloromethane (300 mL). After the addition was complete, the reaction mixture was stirred at room temperature for 3h. The reaction mixture was quenched with aqueous saturated NaHCO₃ solution (2 L) and extracted with dichloromethane (2×2 L). The combined organic layers were dried over anhydrous Na₂SO₄ and concentrated in vacuo to afford N-(2-bromo-5-iodopyridin-4-yl)-N-(methylsulfonyl)methanesulfonamide (Intermediate 5, 150 g, 98%) as a yellow liquid, which was used for next step without further purification. ¹H NMR (400 MHz, DMSO-d₆) δ ppm 3.65 (s, 6H), 8.10 (s, 1H), 8.92 (s, 1H). Mass spec: m/z: 456 [M+H]⁺.

### Step 3

### Intermediate 6: N-(2-bromo-5-iodopyridin-4-yl)methanesulfonamide

To a solution of N-(2-bromo-5-iodopyridin-4-yl)-N-(methylsulfonyl)methanesulfonamide (Intermediate 5, 150 g, 329.6 mmol) in tetrahydrofuran (2 L) and water (2 L) was added sodium hydroxide (79.9 g, 1978 mmol) and the reaction mixture was stirred at room temperature for 3 h. The reaction mixture was poured into water (500 mL), extracted with ethyl acetate (3×1000 mL) and acidified with citric acid solution (1 L) to pH ~4. The resultant precipitate was collected by filtration, washed with water (100 mL) and dried in vacuo to afford N-(2-bromo-5-iodopyridin-4-yl) methanesulfonamide (Intermediate 6, 75 g, 60%) as a yellow solid, which was used for next step without further purification. ¹H NMR (400 MHz, DMSO-d₆) δ ppm 3.28 (s, 3H), 7.54 (s, 1H), 8.64 (s, 1H), 9.51 (s, 1H). Mass spec: m/z: 378.7 [M+H]⁺.

### Step 4

### Intermediate 8: tert-butyl (R)-2-(6-bromo-1-(methylsulfonyl)-1H-pyrrolo[3,2-c]pyridin-2-yl)pyrrolidine-1-carboxylate

To a solution of N-(2-bromo-5-iodopyridin-4-yl)methanesulfonamide (Intermediate 6, 50.0 g, 132.63 mmol) and tert-butyl (R)-2-ethynylpyrrolidine-1-carboxylate (Intermediate 7, 25.89 g, 132.63 mmol) in THF (500 mL) was added DIPEA (138 g, 1061.0 mmol). The reaction mixture was purged with argon gas for 15 min then PdCl₂(PPh₃)₂ (9.60 g, 13.263 mmol) and copper(I) iodide (2.55 g, 13.26 mmol) were added. The reaction mixture was further purged with argon gas for 10 min and then heated at 60° C. for 3 h. The reaction mixture was diluted with water (500 mL) and extracted with ethyl acetate (3×300 mL). The combined organic layers were dried over anhydrous Na₂SO₄ and concentrated in vacuo to obtain crude compound. The crude material was purified by combi-flash chromatography, by eluting with 30% ethyl acetate in heptane, to afford tert-butyl (R)-2-(6-bromo-1-(methylsulfonyl)-1H-pyrrolo[3,2-c]pyridin-2-yl)pyrrolidine-1-carboxylate (Intermediate 8, 38.0 g, 62%) as a yellow solid. ¹H NMR (400 MHz, DMSO-d₆) δ ppm 1.40 (s, 9H), 1.85-1.91 (m, 4H), 3.18-3.21 (m, 1H), 3.37-3.41 (m, 1H), 3.62 (s, 3H), 5.23-5.31 (m, 1H), 6.72 (s, 1H), 7.97 (s, 1H), 8.69 (s, 1H). Mass spec: m/z: 445.9 [M+H]⁺.

### Step 5

### Intermediate 9: (R)-6-bromo-1-(methylsulfonyl)-2-(pyrrolidin-2-yl)-1H-pyrrolo[3,2-c]pyridine hydrochloride

To a cooled (0° C.) solution of tert-butyl-(R)-2-(6-bromo-1-(methylsulfonyl)-1H-pyrrolo[3,2-c]pyridin-2-yl)pyrrolidine-1-carboxylate (Intermediate 8, 200 g, 450.1 mmol) in 1,4-dioxane (1 L) was added 4M HCl in 1,4-dioxane (2 L) and the reaction mixture was stirred at room temperature for 6 h. The reaction mixture was concentrated in vacuo to afford (R)-6-bromo-1-(methylsulfonyl)-2-(pyrrolidin-2-yl)-1H-pyrrolo[3,2-c]pyridine hydrochloride (Intermediate 9, 160 g) as a yellow solid, which was used for the next step without further purification. ¹H NMR (400 MHz, DMSO-d₆) δ ppm 1.87-2.20 (m, 2H), 2.33-2.46 (m, 2H), 3.15-3.34 (m, 2H), 3.75 (s, 3H), 5.12-5.17 (m, 1H), 7.38 (s, 1H), 7.98 (s, 1H), 8.78 (s, 1H), 9.45 (s, 2H). Mass spec: m/z: 345.8 [M+H]⁺.

### Step 6

### Intermediate 10: (R)-6-bromo-2-(1-methylpyrrolidin-2-yl)-1-(methylsulfonyl)-1H-pyrrolo [3,2-c]pyridine

To a cooled (0° C.) solution of (R)-6-bromo-1-(methylsulfonyl)-2-(pyrrolidin-2-yl)-1H-pyrrolo[3,2-c]pyridine hydrochloride (Intermediate 9, 90.0 g, 236.4 mmol) in methanol (900 mL) was added triethylamine (192 g, 1891 mmol) followed by formaldehyde (60.83 g, 709.2 mmol). The reaction mixture was stirred for 5 min and then sodium cyanoborohydride (34.96 g, 472.8 mmol) was added at 0° C. The reaction mixture was then heated at 60° C. for 16 h. The reaction mixture was diluted with water (500 mL) and extracted with dichloromethane (3×500 mL). The combined organic layers were dried over anhydrous Na₂SO₄ and concentrated in vacuo to the obtain crude compound. The crude material was purified by combi-flash chromatography by eluting with 60% ethyl acetate in heptane, to afford (R)-6-bromo-2-(1-methylpyrrolidin-2-yl)-1-(methylsulfonyl)-1H-pyrrolo[3,2-c]pyridine (Intermediate 10, 60.0 g, 71%) as a yellow solid. ¹H NMR (400 MHz, DMSO-d₆) δ ppm 1.70-1.78 (m, 3H), 2.33 (s, 3H), 2.36-2.40 (m, 2H), 3.01-3.22 (m, 1H), 3.56 (s, 3H), 3.76-3.81 (m, 1H), 6.89 (s, 1H), 7.97 (s, 1H), 8.68 (s, 1H). Mass spec: m/z: 359.8 [M+H]⁺.

### Step 7

### Intermediate 11: (R)-6-bromo-2-(1-methylpyrrolidin-2-yl)-1H-pyrrolo[3,2-c]pyridine

To a solution of (R)-6-bromo-2-(1-methylpyrrolidin-2-yl)-1-(methylsulfonyl)-1H-pyrrolo[3,2-c]pyridine (Intermediate 10, 110 g, 307.1 mmol) in methanol (600 mL) was added tetrahydrofuran (600 mL) and the mixture cooled to 0° C. Cesium carbonate (200 g, 614.2 mmol) was added and the reaction mixture was stirred at room temperature for 4 h. The reaction mixture was quenched with water (2 L) and extracted with ethyl acetate (2×1 L). The combined organic layers were dried over anhydrous Na₂SO₄ and concentrated in vacuo to afford (R)-6-bromo-2-(1-methylpyrrolidin-2-yl)-1H-pyrrolo[3,2-c]pyridine (Intermediate 11, 60.0 g, 70%) as a yellow solid, which was used for the next step without further purification. ¹H NMR (400 MHz, DMSO-d₆) δ ppm 1.75-1.91 (m, 3H), 2.14 (s, 3H), 2.21-2.29 (m, 1H), 3.10-3.15 (m, 1H), 3.33-3.36 (m, 2H), 6.45 (s, 1H), 7.43 (s, 1H), 8.48 (s, 1H), 11.57 (br s, 1H). Mass spec: m/z: 279.9 [M+H]⁺.

### Step 8

### Intermediate 1: tert-butyl (R)-6-bromo-2-(1-methylpyrrolidin-2-yl)-1H-pyrrolo[3,2-c]pyridine-1-carboxylate

To a solution of (R)-6-bromo-2-(1-methylpyrrolidin-2-yl)-1H-pyrrolo[3,2-c]pyridine (Intermediate 11, 60.0 g, 214.16 mmol) in dichloromethane (600 mL) was added triethylamine (65.3 g, 642.49 mmol) followed by di-tert-butyldicarbonate (94.42 g, 428.33 mmol) and 4-(dimethylamino)pyridine (5.28 g, 42.833 mmol) and the reaction mixture was stirred at room temperature for 3 h. The reaction mixture was diluted with water (500 mL) and extracted with diethyl ether (3×500 mL). The combined organic layers were dried over anhydrous Na₂SO₄ and concentrated in vacuo to obtain the crude compound. The crude material was purified by combi-flash chromatography, by eluting with 45% ethyl acetate in heptane, followed by SFC purification (Waters SFC Prep 150 Mgm equipped with 2489 PDA detector; Column: Chiralpak IC (30×250 mm, 5 μM); Solvent A) CO₂ 70 g/min; B) 0.1% Isopropyl amine in IPA and MeCN (50:50) 25 mL/min) to afford tert-butyl-(R)-6-bromo-2-(1-methylpyrrolidin-2-yl)-1H-pyrrolo[3,2-c]pyridine-1-carboxylate (Intermediate 1, 43 g, 53%) as an off white solid.

Enantiomeric excess (ee) 98.4%. ¹H NMR (400 MHz, DMSO-d₆) δ ppm 1.65 (s, 9H), 1.71-1.75 (m, 3H), 2.30-2.33 (m, 1H), 2.34 (s, 3H), 2.37-2.40 (m, 1H), 3.11-3.14 (m, 1H), 3.87-3.91 (m, 1H), 6.80 (s, 1H), 8.05 (s, 1H), 8.61 (s, 1H). Mass spec: m/z: 379.9 [M+H]⁺.

### Intermediate 1 can Also be Synthesized Following Scheme 3

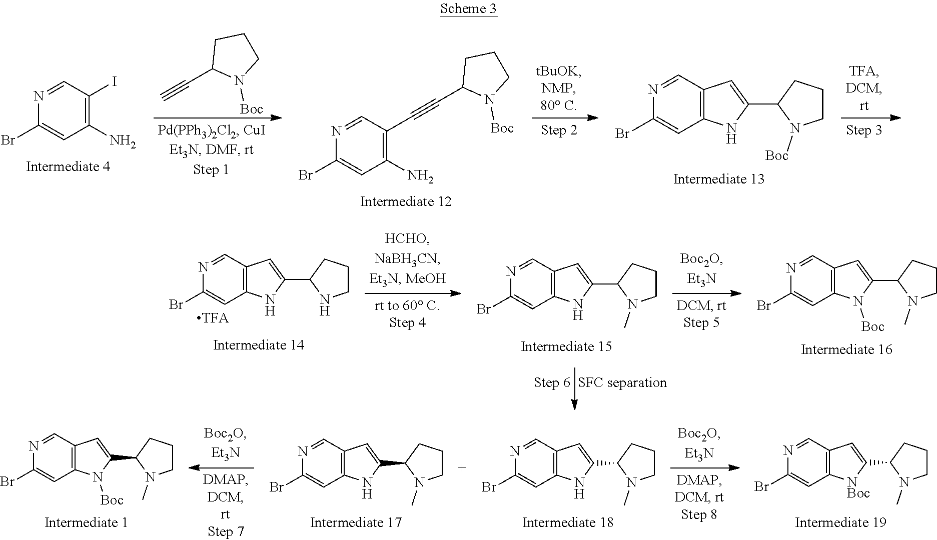

### Step 1

### Intermediate 12: rac-tert-butyl 2-[2-(4-amino-6-bromopyridin-3-yl)ethynyl]pyrrolidine-1-carboxylate

A solution of 2-bromo-5-iodopyridin-4-amine (Intermediate 4: 17 g, 54.2 mmol), rac-tert-butyl 2-ethynylpyrrolidine-1-carboxylate (CAS: 316141-37-4, 13.4 g, 65.04 mmol), Pd(PPh₃)₂Cl₂ (3.81 g, 5.42 mmol), CuI (1.03 g, 5.42 mmol) and Et₃N (16.4 g, 162.6 mmol) in DMF (400 mL) was stirred for 2 h at rt under nitrogen atmosphere. The reaction mixture was quenched with water and the resulting solution was extracted three times with EtOAc. The organic layers were combined, washed with brine, dried over anhydrous Na₂SO₄, filtered, and concentrated under reduced pressure. The crude material was purified by flash chromatography on silica gel using petroleum ether/EtOAc (7/3) as eluent to afford rac-tert-butyl 2-[2-(4-amino-6-bromopyridin-3-yl)ethynyl]pyrrolidine-1-carboxylate (Intermediate 12: 18 g, 83%) as a yellow oil. Mass spec: m/z 366 [M+H]⁺.

### Step 2

### Intermediate 13: rac-tert-butyl 2-{6-bromo-1H-pyrrolo[3,2-c]pyridin-2-yl}pyrrolidine-1-carboxylate

To a solution of rac-tert-butyl 2-[2-(4-amino-6-bromopyridin-3-yl)ethynyl]pyrrolidine-1-carboxylate (Intermediate 12: 8 g, 20.8 mmol) in NMP (40 mL) was added a solution of tBuOK (65.53 mL, 65.53 mmol, 1M in THF). The reaction mixture was stirred for 4 h at 80° C. The reaction mixture was quenched with water and the resulting solution was extracted three times with EtOAc. The organic layers were combined, washed with brine, dried over anhydrous Na₂SO₄, filtered, and concentrated under reduced pressure. The crude material was purified by flash chromatography on silica gel using DCM/MeOH (95/5) as eluent to afford rac-tert-butyl 2-{6-bromo-1H-pyrrolo[3,2-c]pyridin-2-yl}pyrrolidine-1-carboxylate (Intermediate 13: 6 g, 71%) as a yellow oil. Mass spec: m/z 366 [M+H]⁺.

### Step 3

### Intermediate 14: rac-2-{6-bromo-1H-pyrrolo[3,2-c]pyridin-2-yl}pyrrolidine trifluoroacetate

To a solution of rac-tert-butyl 2-{6-bromo-1H-pyrrolo[3,2-c]pyridin-2-yl}pyrrolidine-1-carboxylate (Intermediate 13: 16 g, 41.5 mmol) in DCM (300 mL) was added TFA (30 mL). The reaction mixture was stirred for 2 h at rt. The solution was concentrated under reduced pressure to afford rac-2-{6-bromo-1H-pyrrolo[3,2-c]pyridin-2-yl}pyrrolidine trifluoroacetate (Intermediate 14: 10 g, 77%) as a yellow oil which was used in Step 5 without further purification. Mass spec: m/z 266 [M+H]⁺.

### Step 4

### Intermediate 15: rac-6-bromo-2-(1-methylpyrrolidin-2-yl)-1H-pyrrolo[3,2-c]pyridine

To a solution of rac-6-bromo-2-(pyrrolidin-2-yl)-1H-pyrrolo[3,2-c]pyridine trifluoroacetate (Intermediate 14: 10 g, 35.8 mmol) in MeOH (300 mL) were added Et₃N (10.9 g, 108 mmol) and paraformaldehyde (5.37 g, 179 mmol). The reaction mixture was stirred for 1 h at rt. NaBH₃CN (6.77 g, 107.4 mmol) was added and the reaction mixture was stirred overnight at 60° C. The reaction mixture was quenched with water and the resulting solution was extracted three times with EtOAc. The organic layers were combined, washed with brine, dried over anhydrous Na₂SO₄, filtered, and concentrated under reduced pressure. The residue was chromatographed by reverse-phase flash chromatography (C18 aq) using MeCN/H₂O (45/55) as eluent to afford rac-6-bromo-2-(1-methylpyrrolidin-2-yl)-1H-pyrrolo[3,2-c]pyridine (Intermediate 15: 8 g, 69%) as a yellow oil. Mass spec: m/z 280 [M+H]⁺.

### Step 5

### Intermediate 16: rac-tert-butyl 6-bromo-2-(1-methylpyrrolidin-2-yl)-1H-pyrrolo[3,2-c]pyridine-1-carboxylate

To a solution of rac-6-bromo-2-(1-methylpyrrolidin-2-yl)-1H-pyrrolo[3,2-c]pyridine (Intermediate 15: 88 g, 25.8 mmol) in DCM (200 mL) were added Boc₂O (8.44 g, 38.7 mmol), Et₃N (7.82 g, 77.4 mmol) and DMAP (310 mg, 2.58 mmol). The reaction mixture was stirred overnight at rt and then quenched with water. The resulting solution was extracted three times with EtOAc and the organic layers were combined, washed with brine, dried over anhydrous Na₂SO₄, filtered, and concentrated under reduced pressure. The crude material was purified by flash chromatography on silica gel using EtOAc/petroleum ether (3/2) as eluent to afford rac-tert-butyl 6-bromo-2-(1-methylpyrrolidin-2-yl)-1H-pyrrolo[3,2-c]pyridine-1-carboxylate (Intermediate 16: 8.1 g, 71%) as a yellow oil. Mass spec: m/z 380 [M+H]⁺.

### Step 6

### Intermediate 17: (2R)-2-{6-bromo-1H-pyrrolo[3,2-c]pyridin-2-yl}-1-methylpyrrolidine and Intermediate 18: (2S)-2-{6-bromo-1H-pyrrolo[3,2-c]pyridin-2-yl}-1-methylpyrrolidine

rac-6-bromo-2-(1-methylpyrrolidin-2-yl)-1H-pyrrolo[3,2-c]pyridine (Intermediate 15, 9.00 g, 30.5 mmol) was separated by SFC (Column: CHIRALPAK IH 3*25 cm, 5 m; Mobile Phase A: CO₂, Mobile Phase B: MeOH (+1%-2M NH₃ in MeOH); Flow rate: 85 mL/min; Gradient: isocratic 20% B) to afford (2R)-2-{6-bromo-1H-pyrrolo[3,2-c]pyridin-2-yl}-1-methylpyrrolidine (Intermediate 17, 1st eluting peak, 3.5 g, 37%) as a white solid and (2S)-2-{6-bromo-1H-pyrrolo[3,2-c]pyridin-2-yl}-1-methylpyrrolidine (Intermediate 18, 2nd eluting peak, 4.0 g, 40%) as a white solid.

### Intermediate 17: (2R)-2-{6-bromo-1H-pyrrolo[3,2-c]pyridin-2-yl}-1-methylpyrrolidine

1^{st} eluting peak from SFC purification. Retention time: 4.35 mins.

Enantiomeric excess (ee) 99.8%.

¹H NMR (400 MHz, DMSO-d6) δ ppm 11.56 (s, 1H), 8.49 (s, 1H), 7.43 (s, 1H), 6.45 (s, 1H), 3.34 (t, J=9.0 Hz, 1H), 3.13 (t, J=7.8 Hz, 1H), 2.24-2.30 (m, 1H), 2.15 (s, 4H), 1.87-1.90 (m, 1H), 1.75-1.84 (m, 2H).

### Intermediate 18: (2S)-2-{6-bromo-1H-pyrrolo[3,2-c]pyridin-2-yl}-1-methylpyrrolidine

2nd eluting peak from SFC purification. Retention time: 4.87 mins.

Enantiomeric excess (ee) 99.8%. ¹H NMR (400 MHz, DMSO-d6) δ ppm 11.56 (s, 1H), 8.49 (s, 1H), 7.43 (s, 1H), 6.45 (s, 1H), 3.35 (t, J=11.4 Hz, 1H), 3.14 (t, J=8.0 Hz, 1H), 2.26 (t, J=8.6 Hz, 1H), 2.15 (s, 4H), 1.88 (d, J=8.0 Hz, 1H), 1.80 (d, J=8.0 Hz, 2H).

### Step 7

### Intermediate 1: tert-butyl (R)-6-bromo-2-(1-methylpyrrolidin-2-yl)-1H-pyrrolo[3,2-c]pyridine-1-carboxylate

A 500 mL round-bottom flask was charged with (2R)-2-{6-bromo-1H-pyrrolo[3,2-c]pyridin-2-yl}-1-methylpyrrolidine (Intermediate 17, 500 mg, 1.68 mmol), DCM (10 mL), Et₃N (541 mg, 5.35 mmol), DMAP (21.8 mg, 0.178 mmol), (Boc)₂O (778 mg, 3.57 mmol) and the reaction was stirred overnight at room temperature. The reaction was quenched with water (200 mL). The resulting solution was extracted with ethyl acetate (3×200 mL) and the organic layers were combined, washed with brine (2×300 mL), dried over anhydrous sodium sulfate, filtered, and concentrated under reduced pressure. The residue was chromatographed on a silica gel column, eluting with EtOAc/Petroleum ether (35/65) to afford tert-butyl (R)-6-bromo-2-(1-methylpyrrolidin-2-yl)-1H-pyrrolo[3,2-c]pyridine-1-carboxylate (Intermediate 1, 600 mg, 92%) as an off-white solid. ¹H NMR (400 MHz, DMSO-d6) δ ppm 1.65 (s, 9H), 1.71-1.75 (m, 3H), 2.30-2.33 (m, 1H), 2.34 (s, 3H), 2.37-2.40 (m, 1H), 3.11-3.14 (m, 1H), 3.87-3.91 (m, 1H), 6.80 (s, 1H), 8.05 (s, 1H), 8.61 (s, 1H). Mass spec: m/z: 379.9 [M+H]⁺. Mass spec: m/z: 380.0 [M+H]⁺.

### Step 8

### Intermediate 19: tert-butyl (S)-6-bromo-2-(1-methylpyrrolidin-2-yl)-1H-pyrrolo[3,2-c]pyridine-1-carboxylate

Intermediate 19 was prepared in an analogous manner to Intermediate 1 using (2S)-2-{6-bromo-1H-pyrrolo[3,2-c]pyridin-2-yl}-1-methylpyrrolidine (Intermediate 18) and exhibited the following data: ¹H NMR (400 MHz, DMSO-d6) δ ppm 1.65 (s, 9H), 1.71-1.75 (m, 3H), 2.30-2.33 (m, 1H), 2.34 (s, 3H), 2.37-2.40 (m, 1H), 3.11-3.14 (m, 1H), 3.87-3.91 (m, 1H), 6.80 (s, 1H), 8.05 (s, 1H), 8.61 (s, 1H). Mass spec: m/z: 379.9 [M+H]⁺. Mass spec: m/z: 380.0 [M+H]⁺.

### Intermediate 7: tert-butyl (R)-2-ethynylpyrrolidine-1-carboxylate

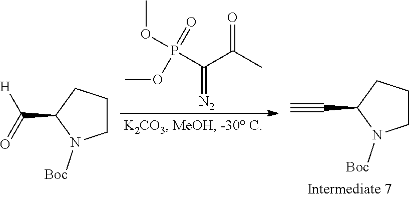

To a cooled (−30° C.) solution of tert-butyl-(R)-2-formylpyrrolidine-1-carboxylate (CAS No: 73365-02-3, 50.0 g, 250.94 mmol) in methanol (500 mL) was added potassium carbonate (51.94 g, 376.41 mmol). Dimethyl(1-diazo-2-oxopropyl)phosphonate (CAS No: 90965-06-3, 57.85 g, 301.13 mmol) was then added dropwise over 20 min and the reaction mixture was stirred at −30° C. for 3 h. The reaction mixture was diluted with water (1000 mL) and extracted with diethyl ether (3×500 mL). The combined organic layers were dried over anhydrous Na₂SO₄ and concentrated in vacuo to obtain the crude compound. The crude material was purified by combi-flash chromatography, by eluting with 10% ethyl acetate in heptane, to afford tert-butyl (R)-2-ethynylpyrrolidine-1-carboxylate (Intermediate 7, 35.0 g, 71%) as a yellow liquid. ¹H NMR (400 MHz, DMSO-d₆) δ ppm 1.40 (s, 9H), 1.81-1.89 (m, 3H), 1.99-2.12 (m, 1H), 3.08-3.25 (m, 2H), 3.26-3.35 (m, 1H), 4.32-4.38 (m, 1H).

### Intermediate 2 was Synthesized Following Scheme 4

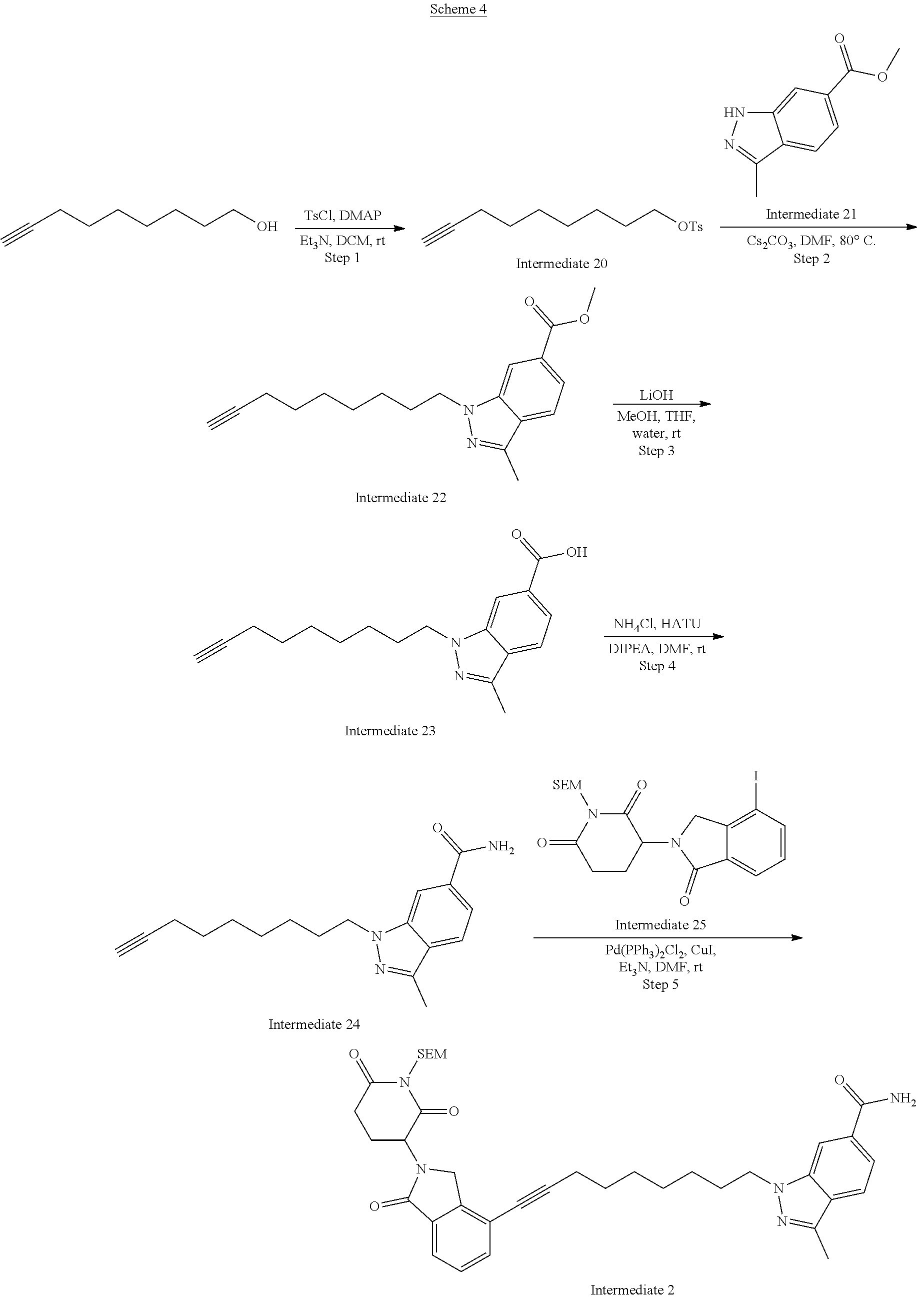

### Step 1

### Intermediate 20: non-8-yn-1-yl 4-methylbenzenesulfonate

To a cooled (0° C.) solution of non-8-yn-1-ol (CAS No: 10160-28-8, 25.0 g, 178.29 mmol) in dichloromethane (250 mL) was added trimethylamine (74.6 mL, 534.87 mmol) followed by 4-dimethylaminopyridine (2.30 g, 17.83 mmol), then p-toluene sulfonyl chloride (1.50 g, 7.9 mmol) and the reaction mixture was stirred at room temperature for 16 h. The reaction mixture was then diluted with water (250 mL) and extracted with dichloromethane (3×250 mL). The combined organic layers were dried over anhydrous Na₂SO₄, and concentrated in vacuo. The crude material was purified by combi-flash chromatography, by eluting with 55% ethyl acetate in heptane, to afford non-8-yn-1-yl 4-methylbenzenesulfonate (Intermediate 20, 34 g, 65%) as a colorless oil. ¹H NMR (400 MHz, DMSO-d₆) δ ppm 1.12-1.30 (m, 6H), 1.32-1.41 (m, 2H), 1.50-1.57 (m, 2H), 2.08-2.13 (m, 2H), 2.42 (s, 3H), 2.71 (s, 1H), 3.98-4.02 (m, 2H), 7.48 (d, J=7.88 Hz, 2H), 7.78 (d, J=8.29 Hz, 2H).

### Step 2

### Intermediate 22: methyl 3-methyl-1-(non-8-yn-1-yl)-1H-indazole-6-carboxylate

To a solution of non-8-yn-1-yl 4-methylbenzenesulfonate (Intermediate 20, 17.03 g, 57.83 mmol) in dimethylformamide (100 mL) was added cesium carbonate (51.4 g, 157.73 mmol) and methyl 3-methyl-1H-indazole-6-carboxylate (Intermediate 21, 10.0 g, 52.57 mmol) at room temperature. The reaction mixture was heated at 80° C. for 2 h then diluted with water (500 mL) and extracted with ethyl acetate (3×200 mL). The combined organic layers were washed with brine solution (2×200 mL), dried over Na₂SO₄, and concentrated in vacuo. The crude material was purified by combi-flash chromatography, by eluting with 30% ethyl acetate in heptane, to afford methyl 3-methyl-1-(non-8-yn-1-yl)-1H-indazole-6-carboxylate (Intermediate 22, 12 g, 67%) as an off-white solid. ¹H NMR (400 MHz, DMSO-d₆) δ ppm 1.18-1.32 (m, 6H), 1.34-1.43 (m, 2H), 1.76-1.83 (m, 2H), 2.09-2.13 (m, 2H), 2.51 (s, 3H), 2.70 (s, 1H), 3.90 (s, 3H), 4.40 (t, J=7.02 Hz, 2H), 7.65 (d, J=7.89 Hz, 1H), 7.81 (d, J=8.33 Hz, 1H), 8.23 (s, 1H). Mass spec: m/z 313.0 [M+H]⁺.

### Step 3

### Intermediate 23: 3-methyl-1-(non-8-yn-1-yl)-1H-indazole-6-carboxylic acid

To a solution of methyl 3-methyl-1-(non-8-yn-1-yl)-1H-indazole-6-carboxylate (Intermediate 22, 21 g, 50.3 mmol) in methanol (100 mL) and tetrahydrofuran (100 mL) and was added lithium hydroxide (4 g, 200 mmol) in water (50 mL) and the reaction mixture was stirred at room temperature for 16 h. The reaction mixture was then concentrated in vacuo. The crude residue was taken up in water (30 mL) and acidified with 0.5 N HCl solution to pH~4. The resultant precipitate was collected by filtration and dried in vacuo to afford 3-methyl-1-(non-8-yn-1-yl)-1H-indazole-6-carboxylic acid (Intermediate 23, 19.0 g, 94%) as a white solid, which was used for the next step without further purification. ¹H NMR (400 MHz, DMSO-d₆) δ ppm 1.26-1.38 (m, 8H), 1.75-1.78 (m, 2H), 2.08-2.11 (m, 2H), 2.52 (s, 3H), 2.70 (s, 1H), 4.33-4.37 (m, 2H), 7.63-7.72 (m, 2H), 8.13 (s, 1H). Mass spec m/z 299 [M+H]⁺.

### Step 4

### Intermediate 24: 3-methyl-1-(non-8-yn-1-yl)-1H-indazole-6-carboxamide

To a solution of 3-methyl-1-(non-8-yn-1-yl)-1H-indazole-6-carboxylic acid (Intermediate 23, 18.0 g, 60.32 mmol) in dimethylformamide (180 mL) was added HATU (35.47 g, 90.48 mmol) and DIPEA (31.6 mL, 181.0 mmol) at room temperature. After stirring for 10 min, ammonium chloride (16.2 g, 301.6 mmol) was added and the reaction mixture was stirred at room temperature for a further 16 h. The reaction mixture was then poured into ice cold water (200 mL), the resultant precipitate was collected by filtration and dried in vacuo to afford 3-methyl-1-(non-8-yn-1-yl)-1H-indazole-6-carboxamide (Intermediate 24, 17.0 g, 95%) as a white solid, which was used for the next step without further purification. ¹H NMR (400 MHz, DMSO-d₆) δ ppm 1.19-1.33 (m, 6H), 1.35-1.44 (m, 2H), 1.75-1.87 (m, 2H), 2.09-2.13 (m, 2H), 2.51 (s, 3H), 2.71 (s, 1H), 4.31-4.35 (m, 2H), 7.42 (br s, 1H), 7.59 (d, J=8.29 Hz, 1H), 7.73 (d, J=8.29 Hz, 1H), 8.04 (br s, 1H), 8.12 (s, 1H). Mass spec m/z 296.0 [M−H]—.

### Step 5

### Intermediate 2: 1-(9-(2-(2,6-dioxo-1-((2-(trimethylsilyl) ethoxy) methyl) piperidin-3-yl)-1-oxoisoindolin-4-yl) non-8-yn-1-yl)-3-methyl-1H-indazole-6-carboxamide

To solution of 3-methyl-1-(non-8-yn-1-yl)-1H-indazole-6-carboxamide (Intermediate 24, 10.0 g, 33.62 mmol) and 3-(4-iodo-1-oxoisoindolin-2-yl)-1-((2-(trimethylsilyl)ethoxy)methyl)piperidine-2,6-dione (Intermediate 25, 16.83 g, 33.62 mmol) in dimethylformamide (100 mL) was added triethylamine (170 mL, 1210 mmol). The reaction mixture was purged with argon gas for 15 min then PdCl₂(PPh₃)₂ (2.38 g, 3.36 mmol) and copper (I) iodide (0.65 g, 3.36 mmol) were added. The reaction mixture was purged with argon gas for another 10 min and then stirred at room temperature for 2 h. The reaction mixture was concentrated in vacuo to obtain the crude compound which was purified by combi-flash chromatography, by eluting with 80% ethyl acetate in heptane, to afford 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 2, 16.0 g, 71%) as a yellow solid. ¹H NMR (400 MHz, DMSO-d₆) δ ppm −0.04 (s, 9H), 0.79-0.86 (m, 2H), 1.24-1.41 (m, 6H), 1.49-1.56 (m, 2H), 1.78-1.85 (m, 2H), 2.01-2.09 (m, 1H), 2.42-2.45 (m, 3H), 2.50 (s, 3H), 2.73-2.76 (m, 2H), 3.01-3.11 (m, 1H), 3.48-3.55 (m, 2H), 4.23-4.27 (m, 1H), 4.30-4.37 (m, 2H), 4.43-4.47 (m, 1H), 5.00-5.08 (m, 2H), 7.41 (br s, 1H), 7.49-7.54 (m, 1H), 7.58-7.63 (m, 2H), 7.69-7.75 (m, 2H), 8.02 (br s, 1H), 8.12 (s, 1H). Mass spec m/z 670.1 [M+H]⁺.

### Intermediate 25 was Synthesised Following Scheme 5

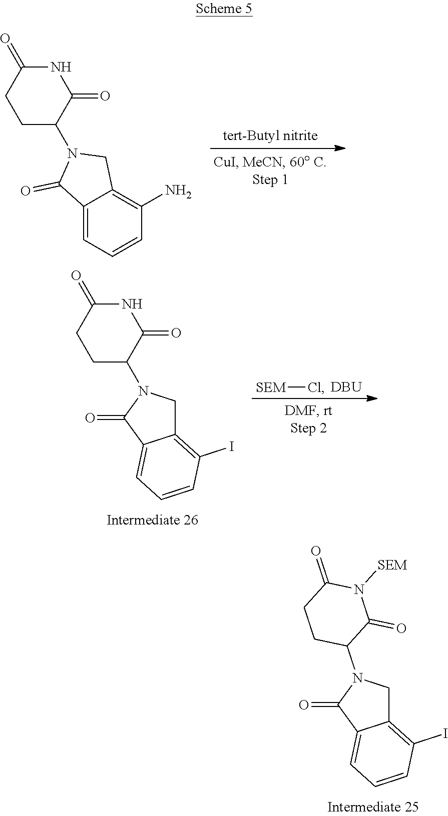

### Step 1

### Intermediate 26: 3-(4-iodo-1-oxoisoindolin-2-yl)piperidine-2,6-dione

To a solution of 3-(4-amino-1-oxoisoindolin-2-yl)piperidine-2,6-dione (CAS No 191732-72-6, 25.0 g, 96.41 mmol) in acetonitrile (300 mL) was added copper(J) iodide (28.10 g, 144.6 mmol). The reaction mixture was purged with argon gas for 20 min then tert-butyl nitrite (16.57 g, 144.6 mmol) was added and the reaction mixture was heated at 60° C. for 12 h. The reaction mixture was poured then into water (300 mL), a solid precipitate formed, which was filtered and dried under vacuum to afford 3-(4-iodo-1-oxoisoindolin-2-yl) piperidine-2,6-dione (Intermediate 26, 45.2 g) as an off white solid, which was used for the next step without further purification. ¹H NMR (400 MHz, DMSO-d6) δ ppm 1.98-2.04 (m, 2H), 2.82-2.98 (m, 2H), 4.11-4.32 (m, 2H), 5.13 (m, 1H), 7.31-7.42 (m, 1H), 7.71-7.80 (m, 1H), 7.98-8.06 (m, 1H), 10.99 (br s, 1H). Mass spec: m/z [M+H]⁺ 371.0.

### Step 2

### Intermediate 25: 3-(4-iodo-1-oxoisoindolin-2-yl)-1-((2-(trimethylsilyl) ethoxy)methyl) piperidine-2,6-dione

To solution of 3-(4-iodo-1-oxoisoindolin-2-yl)piperidine-2,6-dione (Intermediate 24, 30.0 g, 81.06 mmol) in dimethylformamide (250 mL) was added DBU (44.07 g, 283.7 mmol) at 0° C., then 2-(trimethylsilyl)ethoxymethyl chloride (52 mL, 283.7 mmol) was added and the reaction mixture was stirred at room temperature for 16 h under a nitrogen atmosphere. The reaction mixture was quenched with water (700 mL), diluted with ethyl acetate (500 mL) and filtered through a celite bed. The organic layer was separated, washed with water (500 mL), dried over anhydrous Na₂SO₄ and concentrated in vacuo. The crude material was purified by combi-flash chromatography, eluting with 50% ethyl acetate in heptane to afford 3-(4-iodo-1-oxoisoindolin-2-yl)-1-((2-(trimethylsilyl) ethoxy) methyl) piperidine-2,6-dione (Intermediate 25, 8.56 g, 21%) as an off-white solid. ¹H NMR (400 MHz, DMSO-d6) δ ppm −0.02 (s, 9H), 0.78-0.90 (m, 2H), 2.03-2.11 (m, 1H), 2.76-2.82 (m, 1H), 3.02-3.12 (m, 1H), 3.48-3.60 (m, 3H), 4.08-4.13 (m, 1H), 4.28-4.33 (m, 1H), 5.02-5.10 (m, 2H), 5.26-5.31 (m, 1H), 7.31-7.37 (m, 1H), 7.79 (d, J=7.46 Hz, 1H), 8.05 (d, J=7.46 Hz, 1H). Mass spec: m/z [M−H]—498.8.

### Intermediate 21 was Synthesized Following Scheme 6

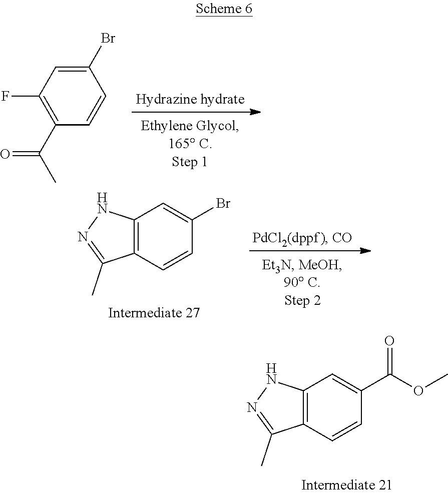

### Step 1

### Intermediate 27: 6-bromo-3-methyl-1H-indazole

To a solution of 1-(4-bromo-2-fluorophenyl)ethan-1-one (CAS No: 625446-22-2, 25.0 g, 115.19 mmol) in ethylene glycol (160.0 mL, 2850 mmol) was added hydrazine hydrate solution (9.18 mL, 287.97 mmol) at room temperature and the reaction mixture was heated at 165° C. for 16h. The reaction mixture was quenched with water (250 mL) resulting in a precipitate which was filtered, washed with water (3×150 mL) and pentane (3×60 mL) and dried under vacuum to afford 6-bromo-3-methyl-1H-indazole (Intermediate 27, 22 g, 90%) as an off-white solid, which was used for the next step without further purification. ¹H NMR (400 MHz, DMSO-d₆) δ ppm 2.47 (s, 3H), 7.19 (d, J=8.80 Hz, 1H), 7.62-7.68 (m, 2H), 12.73 (br s, 1H). Mass spec: m/z: 213.0 [M+H+2]⁺. Step 2

### Intermediate 21: methyl 3-methyl-1H-indazole-6-carboxylate

To a solution of 6-bromo-3-methyl-1H-indazole (Intermediate 27, 15.00 g, 71.07 mmol) in methanol (200 mL) was added triethylamine (29.9 mL, 213.2 mmol) followed by [1,1′-bis(diphenylphosphino)ferrocene]dichloropalladium(II) (5.25 g, 7.11 mmol) at room temperature and the reaction mixture was heated at 90° C. for 16 h under a CO atmosphere (100 psi). The reaction mixture was filtered through celite and concentrated in vacuo to obtain crude compound. The crude material was purified by combi-flash column chromatography by eluting with 25% EtOAc in heptane to afford methyl 3-methyl-1H-indazole-6-carboxylate (Intermediate 21, 10.5 g, 78%) as an off white solid. ¹H NMR (400 MHz, DMSO-d₆) δ ppm 2.52 (s, 3H), 3.88 (s, 3H), 7.64 (d, J=8.32 Hz, 1H), 7.81 (d, J=8.32 Hz, 1H), 8.08 (s, 1H), 13.00 (br s, 1H). Mass spec: m/z: 190.8 [M+H]⁺.

### Example 2 was Synthesized Following Scheme 7

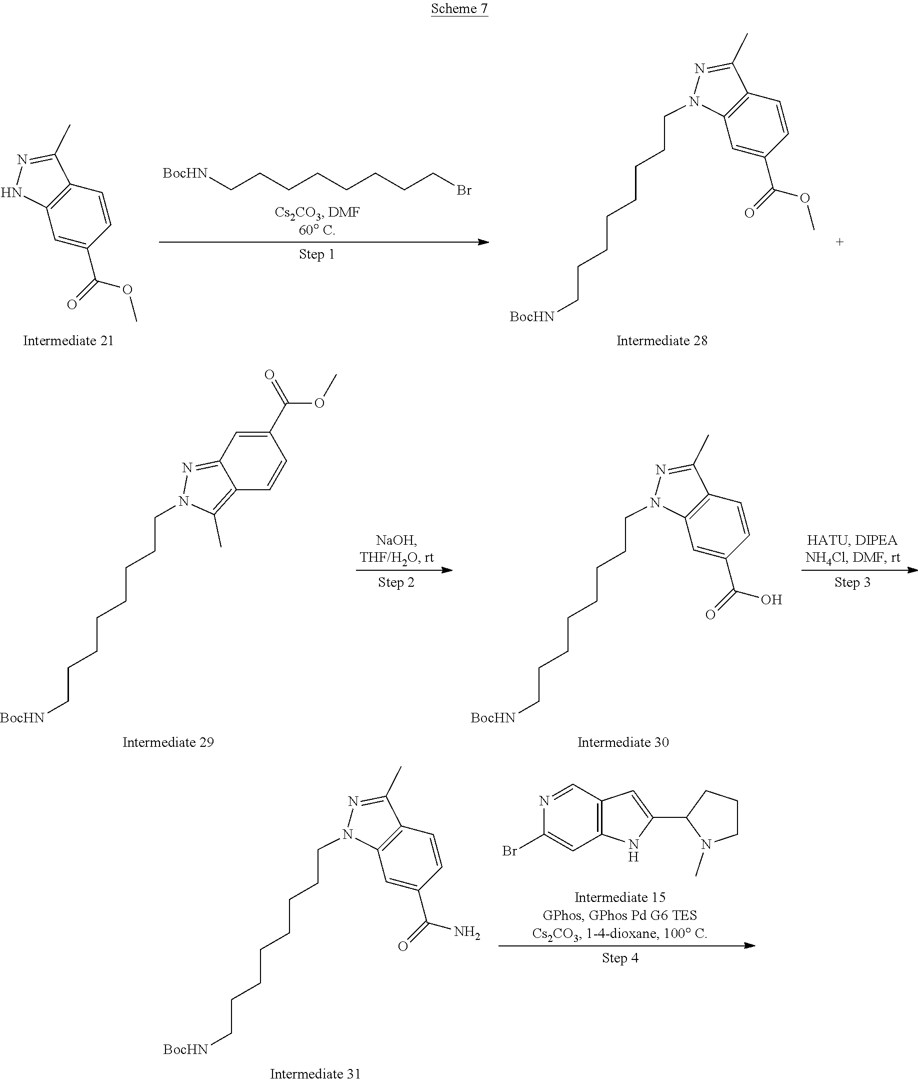

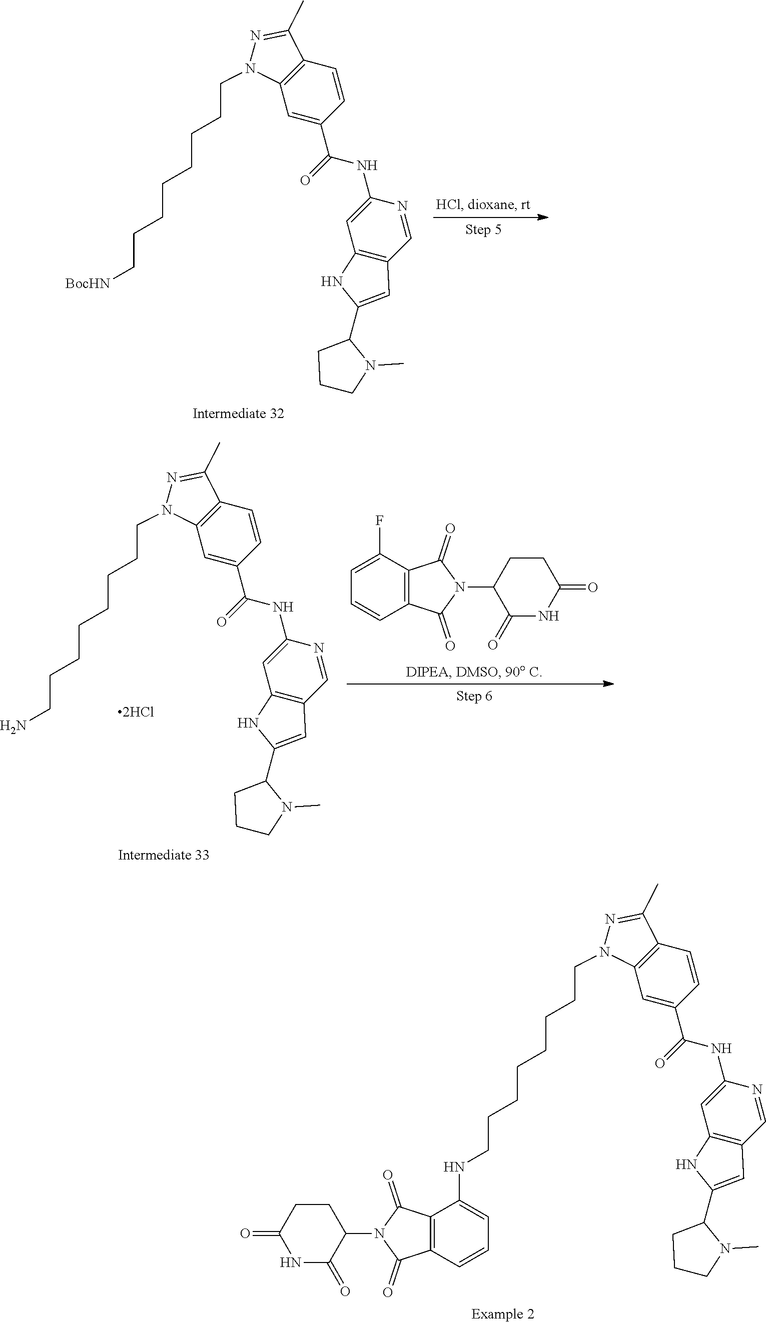 [SMILES: Cc1nn(CCCCCCCCNC(=O)OC(C)(C)C)c2cc(C(=O)Nc3cc4[nH]c(C5CCCN5C)cc4cn3)ccc12 ; Cc1nn(CCCCCCCCN)c2cc(C(=O)Nc3cc4[nH]c(C5CCCN5C)cc4cn3)ccc12 ; O=C1CCC(N2C(=O)c3cccc(F)c3C2=O)C(=O)N1 ; Cc1nn(CCCCCCCCNc2cccc3c2C(=O)N(C2CCC(=O)NC2=O)C3=O)c2cc(C(=O)Nc3cc4[nH]c(C5CCCN5C)cc4cn3)ccc12]

### Step 1

### Intermediate 28: methyl 1-{8-[(tert-butoxycarbonyl)amino]octyl}-3-methylindazole-6-carboxylate and Intermediate 29: methyl 2-(8-((tert-butoxycarbonyl)amino)octyl)-3-methyl-2H-indazole-6-carboxylate

To a solution of methyl 3-methyl-1H-indazole-6-carboxylate (Intermediate 21, 12.0 g, 56.8 mmol) in DMF (150 mL) was added tert-butyl (8-bromooctyl)carbamate (21.0 g, 68.2 mmol) and Cs₂CO₃ (55.5 g, 170 mmol). and the mixture was stirred at 60° C. overnight. Water (200 mL) was added and the resulting solution was extracted with ethyl acetate (3×400 mL) and the organic layers were combined, washed with brine (1000 mL), dried over anhydrous sodium sulfate, filtered and concentrated under reduced pressure. The residue was chromatographed on a silica gel column, eluting with ethyl acetate/petroleum ether (65/35), to afford methyl 1-(8-((tert-butoxycarbonyl)amino)octyl)-3-methyl-1H-indazole-6-carboxylate (Intermediate 28, 20.0 g, 76%) as a yellow oil and methyl 2-(8-((tert-butoxycarbonyl)amino)octyl)-3-methyl-2H-indazole-6-carboxylate (Intermediate 29, 7.0 g, 27%) as a yellow oil.

Intermediate 28 exhibited: ¹H NMR (400 MHz, DMSO-d6) δ ppm 8.20 (s, 1H), 7.78 (d, J=8.4 Hz, 1H), 7.64 (d, J=8.4 Hz, 1H), 6.72 (br, 1H), 4.37 (t, J=6.8 Hz, 2H), 3.90 (s, 3H), 2.85-2.87 (m, 2H), 1.78-1.79 (m, 2H), 1.30-1.35 (m, 13H), 1.17-1.22 (m, 9H). Mass spec: m/z: 418.0 [M+H]⁺.

Intermediate 29 exhibited: ¹H NMR (400 MHz, DMSO-d6) δ ppm 8.21 (s, 1H), 7.78 (d, J=8.8 Hz, 1H), 7.49 (d, J=8.8 Hz, 1H), 6.74 (t, J=5.2 Hz, 1H), 4.38 (t, J=7.2 Hz, 2H), 3.87 (s, 3H), 2.87-2.88 (m, 2H), 2.65 (s, 3H), 1.86 (br, 2H), 1.32-1.36 (m, 10H), 1.22-1.28 (m, 9H).

Mass spec: m/z: 418.0 [M+H]⁺.

### Step 2

### Intermediate 30: 1-{8-[(tert-butoxycarbonyl)amino]octyl}-3-methylindazole-6-carboxylic acid

To a solution of methyl 1-{8-[(tert-butoxycarbonyl)amino]octyl}-3-methylindazole-6-carboxylate (Intermediate 28: 500 mg, 1.08 mmol) in H₂O (30 mL)/THF (30 mL) was added NaOH (3.00 g, 75.0 mmol). The reaction mixture was stirred at rt for 2h, followed by addition of water. The solution was adjusted to pH=6 with a solution of HCl (12N in H₂O). The mixture was extracted three times with EtOAc and the combined organic layers were washed with brine, dried over anhydrous Na₂SO₄, filtered, and concentrated under reduced pressure to afford 1-{8-[(tert-butoxycarbonyl)amino]octyl}-3-methylindazole-6-carboxylic acid (Intermediate 30: 451 mg, 91%) which was used in Step 3 without further purification. Mass spec: m/z: 404 [M+H]⁺.

### Step 3

### Intermediate 31: tert-butyl N-[8-(6-carbamoyl-3-methylindazol-1-yl)octyl]carbamate

To a solution of 1-{8-[(tert-butoxycarbonyl)amino]octyl}-3-methylindazole-6-carboxylic acid (Intermediate 30: 451 mg, 1.13 mmol) in DMF (15 mL) were added NH₄Cl (265 mg, 5.00 mmol), DIPEA (961 mg, 7.43 mmol) and HATU (612 mg, 1.61 mmol). The reaction mixture was stirred at rt overnight and quenched with water. The mixture was extracted three times with EtOAc and the combined organic layers were washed with brine, dried over anhydrous Na₂SO₄, filtered, and concentrated under reduced pressure to afford tert-butyl N-[8-(6-carbamoyl-3-methylindazol-1-yl)octyl]carbamate (Intermediate 31: 265 mg, 65%) as a yellow solid which was used in Step 4 without further purification. Mass spec: m/z: 403 [M+H]⁺.

### Step 4

### Intermediate 32: rac-tert-butyl N-[8-(3-methyl-6-{[2-(1-methylpyrrolidin-2-yl)-1H-pyrrolo[3,2-c]pyridin-6-yl]carbamoyl}indazol-1-yl)octyl]carbamate

To a solution of tert-butyl N-[8-(6-carbamoyl-3-methylindazol-1-yl)octyl]carbamate (Intermediate 31: 230 mg, 0.496 mmol) in 1,4-dioxane (8 mL) were added rac-2-{6-bromo-1H-pyrrolo[3,2-c]pyridin-2-yl}-1-methylpyrrolidine (Intermediate 15: 160 mg, 0.513 mmol), Cs₂CO₃ (558 mg, 1.71 mmol), GPhos Pd G6 TES (54.0 mg, 0.0570 mmol) and GPhos (30.7 mg, 0.0570 mmol). The reaction mixture was stirred at 100° C. for 3 h under nitrogen atmosphere and then quenched with water. The mixture was extracted three times with EtOAc, the combined organic layers were washed with brine, dried over anhydrous Na₂SO₄, filtered, and concentrated under reduced pressure. The crude material was purified by flash chromatography on silica gel using DCM/MeOH (85/15) as eluent to afford rac-tert-butyl N-[8-(3-methyl-6-{[2-(1-methylpyrrolidin-2-yl)-1H-pyrrolo[3,2-c]pyridin-6-yl]carbamoyl}indazol-1-yl)octyl]carbamate (Intermediate 32: 230 mg, 77%) as a yellow solid. Mass spec: m/z: 602 [M+H]⁺.

### Step 5

### Intermediate 33: rac-1-(8-aminooctyl)-3-methyl-N-[2-(1-methylpyrrolidin-2-yl)-1H-pyrrolo[3,2-c]pyridin-6-yl]indazole-6-carboxamide dihydrochloride

To a solution of rac-tert-butyl N-[8-(3-methyl-6-{[2-(1-methylpyrrolidin-2-yl)-1H-pyrrolo[3,2-c]pyridin-6-yl]carbamoyl}indazol-1-yl)octyl]carbamate (Intermediate 32: 160 mg, 0.256 mmol) in 1,4-dioxane (2 mL) was added a solution of HCl (1 mL, 12N in H₂O). The reaction mixture was stirred at rt for 2 h and concentrated under reduced pressure to afford rac-1-(8-aminooctyl)-3-methyl-N-[2-(1-methylpyrrolidin-2-yl)-1H-pyrrolo[3,2-c]pyridin-6-yl]indazole-6-carboxamide dihydrochloride (Intermediate 33: 130 mg, 96%) as a yellow solid. Mass spec: m/z: 502 [M+H]⁺.

### Step 6

### Example 2: rac-1-(8-{[2-(2,6-dioxopiperidin-3-yl)-1,3-dioxoisoindol-4-yl]amino}octyl)-3-methyl-N-[2-(1-methylpyrrolidin-2-yl)-1H-pyrrolo[3,2-c]pyridin-6-yl]indazole-6-carboxamide

To a solution of rac-1-(8-aminooctyl)-3-methyl-N-[2-(1-methylpyrrolidin-2-yl)-1H-pyrrolo[3,2-c]pyridin-6-yl]indazole-6-carboxamide hydrochloride (Intermediate 33: 130 mg, 0.247 mmol) in DMF (5 mL) were added 2-(2,6-dioxopiperidin-3-yl)-4-fluoroisoindole-1,3-dione (CAS: 835616-60-9, 71.6 mg, 0.260 mmol) and K₂CO₃ (143 mg, 1.04 mmol). The reaction mixture was stirred at 90° C. overnight and then quenched with water. The mixture was extracted three times with EtOAc, the combined organic layers were washed with brine, dried over anhydrous Na₂SO₄, filtered, and concentrated under reduced pressure. The crude material was purified by flash chromatography on silica gel using DCM/MeOH (95/5) as eluent, followed by a second purification using preparative HPLC (column: Kinetex EVO C18 21.2×250 mm, 5.0 um; Eluent: H₂O (10 mmol/L NH₄HCO₃)/MeCN from 49/51 to 19/81; Flow rate: 25 mL/min) to afford rac-1-(8-{[2-(2,6-dioxopiperidin-3-yl)-1,3-dioxoisoindol-4-yl]amino}octyl)-3-methyl-N-[2-(1-methylpyrrolidin-2-yl)-1H-pyrrolo[3,2-c]pyridin-6-yl]indazole-6-carboxamide (Example 2: 9.6 mg, 5%) as a yellow solid. Mass spec: m/z: 758 [M+H]⁺; H NMR (300 MHz, MeOH-d₄) δ ppm 8.52 (s, 1H), 8.20 (d, J=6.6 Hz, 2H), 7.81-7.84 (m, 1H), 7.70-7.74 (m, 1H), 7.44-7.49 (m, 1H), 6.93-6.98 (m, 2H), 6.51 (s, 1H), 4.40-4.44 (m, 1H), 3.20-3.64 (m, 5H), 2.70-2.85 (m, 3H), 2.58 (s, 3H), 2.35-2.38 (m, 1H), 2.29 (s, 3H), 1.89-2.09 (m, 6H), 1.55-1.57 (m, 2H), 1.28-1.32 (m, 9H).

### Example 3 was Synthesised Following Scheme 8

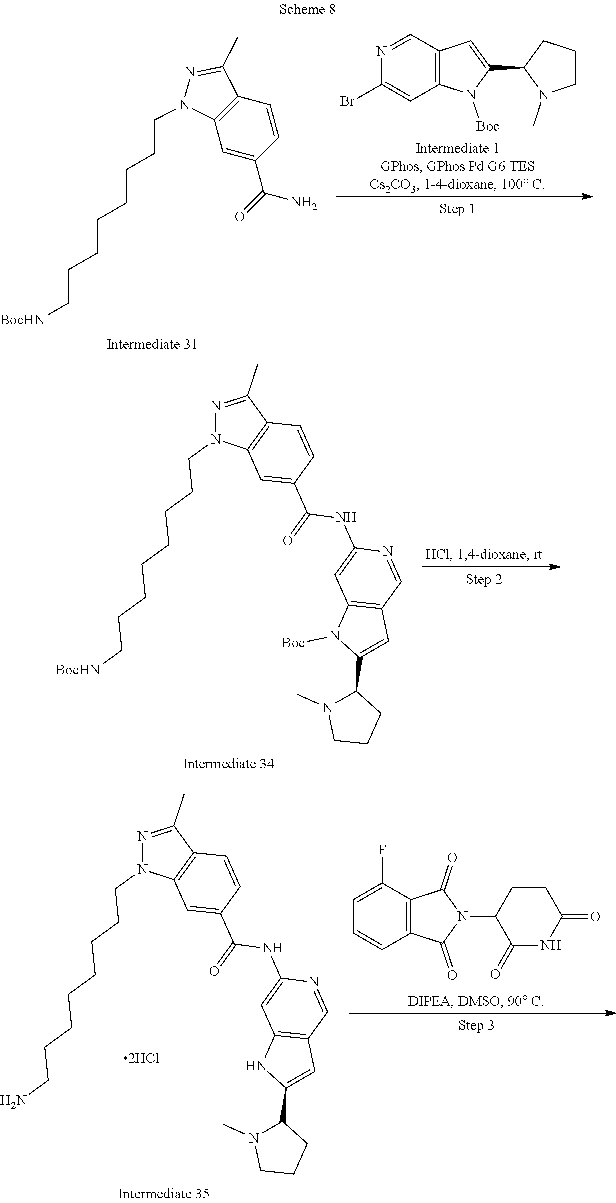

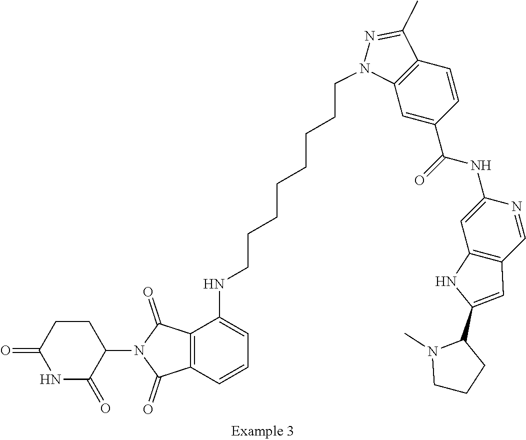

### Step 1

### Intermediate 34: tert-butyl 6-(1-{8-[(tert-butoxycarbonyl)amino]octyl}-3-methylindazole-6-amido)-2-[(2R)-1-methylpyrrolidin-2-yl]pyrrolo[3,2-c]pyridine-1-carboxylate

To a solution of tert-butyl N-[8-(6-carbamoyl-3-methylindazol-1-yl)octyl]carbamate (Intermediate 31: 200 mg, 0.621 mmol) in 1,4-dioxane (6.00 ml) were added tert-butyl 6-bromo-2-[(2R)-1-methylpyrrolidin-2-yl]pyrrolo[3,2-c]pyridine-1-carboxylate (Intermediate 1: 212 mg, 0.559 mmol), Cs₂CO₃ (1.01 g, 3.11 mmol) followed by Gphos (31.9 mg, 0.106 mmol) and Gphos Pd G6 TES (43.3 mg, 0.154 mmol). The reaction mixture was stirred at 100° C. for 3 h under nitrogen atmosphere and quenched with water. The mixture was extracted three times with EtOAc, the combined organic layers were washed with brine, dried over anhydrous Na₂SO₄, filtered, and concentrated under reduced pressure. The crude material was purified by flash chromatography on silica gel using DCM/MeOH (95/5) as eluent to afford tert-butyl 6-(1-{8-[(tert-butoxycarbonyl)amino]octyl}-3-methylindazole-6-amido)-2-[(2R)-1-methylpyrrolidin-2-yl]pyrrolo[3,2-c]pyridine-1-carboxylate (Intermediate 34: 250 mg, 75%) as an off-white solid. Mass spec: m/z: 602 [M+H-Boc]+.

### Step 2

### Intermediate 35: 1-(8-aminooctyl)-3-methyl-N-{2-[(2R)-1-methylpyrrolidin-2-yl]-1H-pyrrolo[3,2-c]pyridin-6-yl}indazole-6-carboxamide dihydrochloride

To a solution of tert-butyl 6-(1-{8-[(tert-butoxycarbonyl)amino]octyl}-3-methylindazole-6-amido)-2-[(2R)-1-methylpyrrolidin-2-yl]pyrrolo[3,2-c]pyridine-1-carboxylate (Intermediate 34: 250 mg, 0.299 mmol) in 1,4-dioxane (3 mL) was added a solution of HCl (1.5 mL, 12N in H₂O). The reaction mixture was stirred at rt for 2 h and concentrated under reduced pressure to afford 1-(8-aminooctyl)-3-methyl-N-{2-[(2R)-1-methylpyrrolidin-2-yl]-1H-pyrrolo[3,2-c]pyridin-6-yl}indazole-6-carboxamide dihydrochloride (Intermediate 35: 170 mg, 95%) as an off-white solid. Mass spec: m/z: 502 [M+H]⁺.

### Step 4

### Example 3: 1-(8-{[2-(2,6-dioxopiperidin-3-yl)-1,3-dioxoisoindol-4-yl]amino}octyl)-3-methyl-N-{2-[(2R)-1-methylpyrrolidin-2-yl]-1H-pyrrolo[3,2-c]pyridin-6-yl}indazole-6-carboxamide

To a solution of 1-(8-aminooctyl)-3-methyl-N-{2-[(2R)-1-methylpyrrolidin-2-yl]-1H-pyrrolo[3,2-c]pyridin-6-yl}indazole-6-carboxamide dihydrochloride (Intermediate 35: 150 mg, 0.299 mmol) in DMSO (1 mL) were added 2-(2,6-dioxopiperidin-3-yl)-4-fluoroisoindole-1,3-dione (CAS: 835616-60-9, 165 mg, 0.598 mmol) and DIPEA (231 mg, 1.79 mmol). The reaction mixture was stirred at 90° C. overnight and concentrated under reduced pressure. The crude material was purified by preparative HPLC (Column: XBridge OBD, 30×150 mm, 5 μm; Eluent: H₂O (10 mmol/L NH₄HCO₃)/MeCN from 1/1 to 20/80; Flow rate: 60 mL/min) to afford 1-(8-{[2-(2,6-dioxopiperidin-3-yl)-1,3-dioxoisoindol-4-yl]amino}octyl)-3-methyl-N-{2-[(2R)-1-methylpyrrolidin-2-yl]-1H-pyrrolo[3,2-c]pyridin-6-yl}indazole-6-carboxamide (Example 3: 45.1 mg, 20%) as a yellow solid. ¹H NMR (400 MHz, DMSO-d₆) δ ppm 11.43 (s, 1H), 11.01-11.09 (m, 1H), 10.61 (s, 1H), 8.51 (s, 1H), 8.47 (s, 1H), 8.28 (s, 1H), 7.73-7.82 (m, 2H), 7.53-7.59 (m, 1H), 6.92-7.06 (m, 2H), 6.53-6.59 (m, 1H), 6.41 (s, 1H), 5.02-5.09 (m, 1H), 4.36-4.58 (m, 2H), 3.32 (s, 3H), 3.13-3.17 (m, 1H), 2.84-2.88 (m, 1H), 2.67-2.72 (m, 4H), 2.24-2.28 (m, 1H), 2.14-2.17 (m, 4H), 1.99-2.03 (m, 1H), 1.74-1.90 (m, 5H), 1.53-1.62 (m, 2H), 1.21-1.36 (m, 9H). Mass spec: m/z: 758 [M+H]⁺.

### Example 4: 1-(8-{[2-(2,6-dioxopiperidin-3-yl)-1,3-dioxoisoindol-4-yl]amino}octyl)-3-methyl-N-{2-[(2S)-1-methylpyrrolidin-2-yl]-1H-pyrrolo[3,2-c]pyridin-6-yl}indazole-6-carboxamide

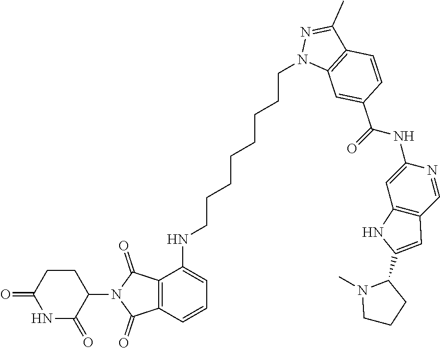 [SMILES: Cc1nn(CCCCCCCCNc2cccc3c2C(=O)N(C2CCC(=O)NC2=O)C3=O)c2cc(C(=O)Nc3cc4[nH]c([C@@H]5CCCN5C)cc4cn3)ccc12]

Following an analogous procedure to Scheme 8, Step 3, using 1-(8-aminooctyl)-3-methyl-N-{2-[(2S)-1-methylpyrrolidin-2-yl]-1H-pyrrolo[3,2-c]pyridin-6-yl}indazole-6-carboxamide dihydrochloride (Intermediate 36: 50 mg, 0.119 mmol), after purification by reverse-phase flash chromatography (C18) using MeCN/H₂O (80/20) as eluent, afforded 1-(8-{[2-(2,6-dioxopiperidin-3-yl)-1,3-dioxoisoindol-4-yl]amino}octyl)-3-methyl-N-{2-[(2S)-1-methylpyrrolidin-2-yl]-1H-pyrrolo[3,2-c]pyridin-6-yl}indazole-6-carboxamide (Example 4: 26.4 mg, 35%) as a yellow solid. Mass spec: m/z: 758 [M+H]⁺. H NMR (400 MHz, DMSO-d₆) δ ppm 11.41 (s, 1H), 11.09 (s, 1H), 10.56 (s, 1H), 8.53 (s, 1H), 8.44 (s, 1H), 8.26 (s, 1H), 7.72-7.79 (m, 2H), 7.54-7.58 (m, 1H), 6.99-7.05 (m, 2H), 6.43-6.53 (m, 2H), 5.02-5.06 (m, 1H), 4.38-4.41 (m, 2H), 3.17 (s, 3H), 2.84-2.87 (m, 1H), 2.51-2.60 (m, 5H), 2.01-2.31 (m, 6H), 1.89-2.02 (m, 5H), 1.57-1.61 (m, 2H), 1.87-1.91 (m, 9H).

### Intermediate 36: 1-(8-aminooctyl)-3-methyl-N-{2-[(2S)-1-methylpyrrolidin-2-yl]-1H-pyrrolo[3,2-c]pyridin-6-yl}indazole-6-carboxamide dihydrochloride

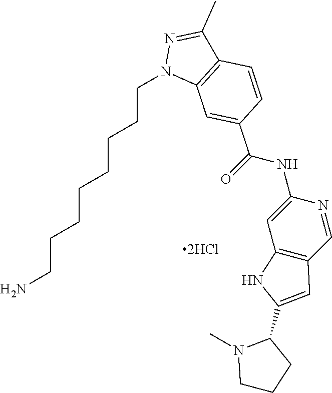 [SMILES: Cc1nn(CCCCCCCCN)c2cc(C(=O)Nc3cc4[nH]c([C@@H]5CCCN5C)cc4cn3)ccc12]

Following an analogous procedure to Scheme 8, Step 2, using tert-butyl 6-(1-{8-[(tert-butoxycarbonyl)amino]octyl}-3-methylindazole-6-amido)-2-[(2S)-1-methylpyrrolidin-2-yl]pyrrolo[3,2-c]pyridine-1-carboxylate (Intermediate 37, 120 mg, 0.141 mmol), afforded 1-(8-aminooctyl)-3-methyl-N-{2-[(2S)-1-methylpyrrolidin-2-yl]-1H-pyrrolo[3,2-c]pyridin-6-yl}indazole-6-carboxamide dihydrochloride (Intermediate 36: 50 mg, 90%) as an off-white solid. Mass spec: m/z: 502 [M+H]⁺.

### Intermediate 37: tert-butyl 6-(1-{8-[(tert-butoxycarbonyl)amino]octyl}-3-methylindazole-6-amido)-2-[(2S)-1-methylpyrrolidin-2-yl]pyrrolo[3,2-c]pyridine-1-carboxylate

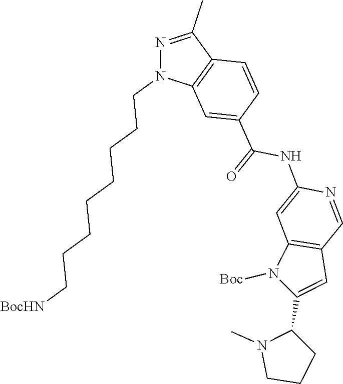 [SMILES: Cc1nn(CCCCCCCCNC(=O)OC(C)(C)C)c2cc(C(=O)Nc3cc4c(cn3)cc([C@@H]3CCCN3C)n4C(=O)OC(C)(C)C)ccc12]

Following an analogous procedure to Scheme 8, Step 1, using tert-butyl N-[8-(6-carbamoyl-3-methylindazol-1-yl)octyl]carbamate (Intermediate 31: 190 mg, 0.472 mmol) and tert-butyl 6-bromo-2-[(2S)-1-methylpyrrolidin-2-yl]pyrrolo[3,2-c]pyridine-1-carboxylate (Intermediate 19: 162 mg, 0.424 mmol), afforded tert-butyl 6-(1-{8-[(tert-butoxycarbonyl)amino]octyl}-3-methylindazole-6-amido)-2-[(2S)-1-methylpyrrolidin-2-yl]pyrrolo[3,2-c]pyridine-1-carboxylate (Intermediate 37: 120 mg, 25%) as an off-white solid. Mass spec: m/z: 602 [M+H-Boc]+.

### Example 5 was Synthesised Following Scheme 9

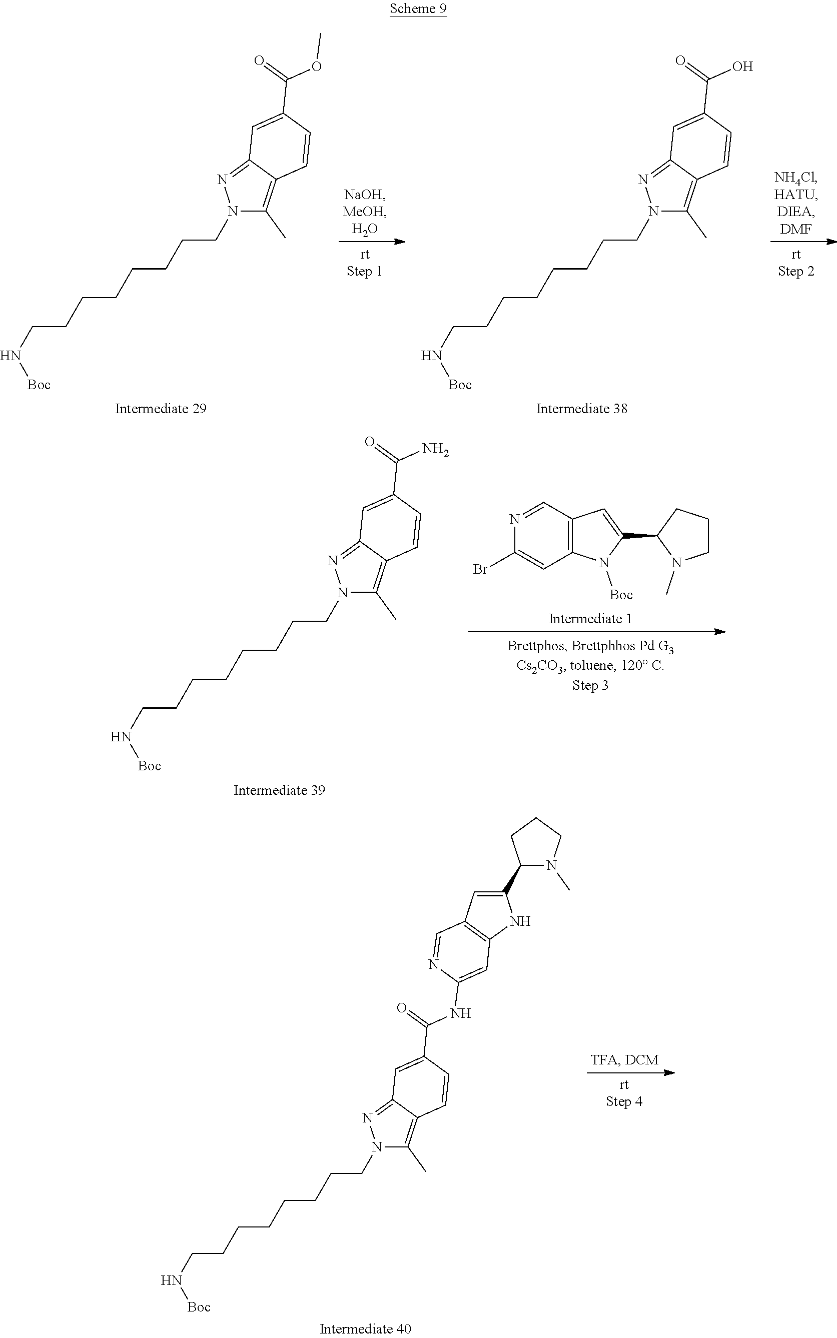 [SMILES: COC(=O)c1ccc2c(C)n(CCCCCCCCNC(=O)OC(C)(C)C)nc2c1 ; Cc1c2ccc(C(=O)O)cc2nn1CCCCCCCCNC(=O)OC(C)(C)C ; Cc1c2ccc(C(N)=O)cc2nn1CCCCCCCCNC(=O)OC(C)(C)C ; CN1CCC[C@@H]1c1cc2cnc(Br)cc2n1C(=O)OC(C)(C)C ; Cc1c2ccc(C(=O)Nc3cc4[nH]c([C@H]5CCCN5C)cc4cn3)cc2nn1CCCCCCCCNC(=O)OC(C)(C)C]

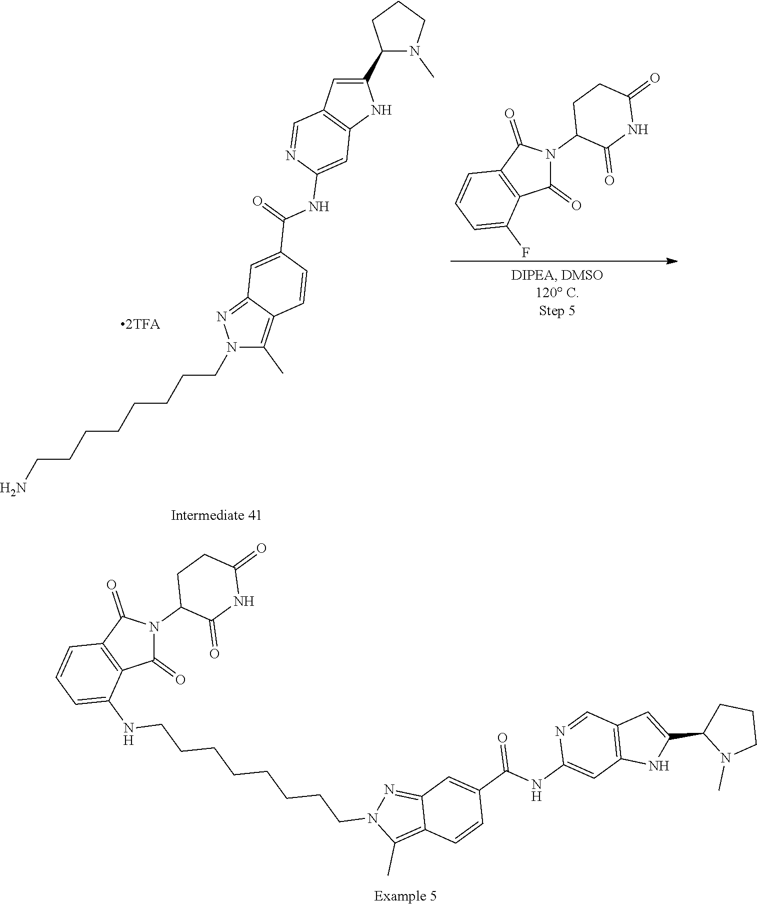 [SMILES: Cc1c2ccc(C(=O)Nc3cc4[nH]c([C@H]5CCCN5C)cc4cn3)cc2nn1CCCCCCCCN ; O=C1CCC(N2C(=O)c3cccc(F)c3C2=O)C(=O)N1 ; Cc1c2ccc(C(=O)Nc3cc4[nH]c([C@H]5CCCN5C)cc4cn3)cc2nn1CCCCCCCCNc1cccc2c1C(=O)N(C1CCC(=O)NC1=O)C2=O]

### Step 1

### Intermediate 38: 2-(8-((tert-butoxycarbonyl)amino)octyl)-3-methyl-2H-indazole-6-carboxylic acid

To a solution of methyl 2-(8-((tert-butoxycarbonyl)amino)octyl)-3-methyl-2H-indazole-6-carboxylate (Intermediate 29, 500 mg, 1.14 mmol, 95%) in MeOH (10 mL) was added NaOH (800 mg, 21.1 mmol) in H₂O (10 mL) and the mixture was stirred at room temperature for 3h. The pH of the mixture was adjusted to 6 by the addition of HCl (1M). The resulting solution was extracted with ethyl acetate (3×50 mL) and the organic layers were combined, washed with brine (200 mL), dried over anhydrous sodium sulfate, filtered and concentrated under reduced pressure to afford 2-(8-((tert-butoxycarbonyl)amino)octyl)-3-methyl-2H-indazole-6-carboxylic acid (Intermediate 38, 480 mg, 99%) as a yellow solid, which was used in the next step without further purification. Mass spec: m/z 404.3 [M+H]⁺.

### Step 2

### Intermediate 39: tert-butyl (8-(6-carbamoyl-3-methyl-2H-indazol-2-yl)octyl)carbamate

To a solution of 2-(8-((tert-butoxycarbonyl)amino)octyl)-3-methyl-2H-indazole-6-carboxylic acid (Intermediate 38, 250 mg, 0.589 mmol) in DMF (5 mL) was added NH₄Cl (126 mg, 2.36 mmol), HATU (336 mg, 0.883 mmol) and DIPEA (456 mg, 3.53 mmol) and the mixture was stirred at room temperature for 3h. Water (10 mL) was added and the resulting solution was extracted with ethyl acetate (3×20 mL). The organic layers were combined, washed with brine (100 mL), dried over anhydrous sodium sulfate, filtered and concentrated under reduced pressure. The residue was chromatographed on a silica gel column, eluting with methanol/dichloromethane (5/95), to afford tert-butyl (8-(6-carbamoyl-3-methyl-2H-indazol-2-yl)octyl)carbamate (Intermediate 39, 240 mg, 98%) as yellow oil. Mass spec: m/z 403.2 [M+H]⁺.

### Step 3

### Intermediate 40: tert-butyl (R)-(8-(3-methyl-6-((2-(1-methylpyrrolidin-2-yl)-1H-pyrrolo[3,2-c]pyridin-6-yl)carbamoyl)-2H-indazol-2-yl)octyl)carbamate

To a solution of tert-butyl (8-(6-carbamoyl-3-methyl-2H-indazol-2-yl)octyl)carbamate (Intermediate 39, 200 mg, 0.482 mmol) in toluene (10 mL) was added tert-butyl 6-bromo-2-[(2R)-1-methylpyrrolidin-2-yl]pyrrolo[3,2-c]pyridine-1-carboxylate (Intermediate 1, 183 mg, 0.482 mmol), Brettphos (155 mg, 0.289 mmol), Brettphos Pd G₃ (131 mg, 0.145 mmol) and Cs₂CO₃ (314 mg, 0.964 mmol) and the mixture was stirred at 120° C. for overnight under nitrogen. The reaction was then concentrated under reduced pressure and the resulting residue was chromatographed on a silica gel column, eluting with MeOH/DCM (4/96), to afford tert-butyl (R)-(8-(3-methyl-6-((2-(1-methylpyrrolidin-2-yl)-1H-pyrrolo[3,2-c]pyridin-6-yl)carbamoyl)-2H-indazol-2-yl)octyl)carbamate (Intermediate 40, 150 mg, 41%) as yellow oil. Mass spec: m/z: 702.4 [M+H]⁺.

### Step 4

### Intermediate 41: (R)-2-(8-aminooctyl)-3-methyl-N-(2-(1-methylpyrrolidin-2-yl)-1H-pyrrolo[3,2-c]pyridin-6-yl)-2H-indazole-6-carboxamide ditrifluoroacetate

To a solution of tert-butyl (R)-(8-(3-methyl-6-((2-(1-methylpyrrolidin-2-yl)-1H-pyrrolo[3,2-c]pyridin-6-yl)carbamoyl)-2H-indazol-2-yl)octyl)carbamate (Intermediate 40, 150 mg, 0.197 mmol) in DCM (2 mL) was added TFA (1 mL) and the mixture was stirred at room temperature for 1 h. The volatiles were removed under reduced pressure to afford (R)-2-(8-aminooctyl)-3-methyl-N-(2-(1-methylpyrrolidin-2-yl)-1H-pyrrolo[3,2-c]pyridin-6-yl)-2H-indazole-6-carboxamide ditrifluoroacetate (Intermediate 41, 100 mg, 97%) as yellow oil, which was used in the next step without further purification. Mass spec: m/z: 502.3 [M+H]⁺.

### Step 5

### Example 5: 2-(8-((2-(2,6-dioxopiperidin-3-yl)-1,3-dioxoisoindolin-4-yl)amino)octyl)-3-methyl-N-(2-((R)-1-methylpyrrolidin-2-yl)-1H-pyrrolo[3,2-c]pyridin-6-yl)-2H-indazole-6-carboxamide

To a solution of(R)-2-(8-aminooctyl)-3-methyl-N-(2-(1-methylpyrrolidin-2-yl)-1H-pyrrolo[3,2-c]pyridin-6-yl)-2H-indazole-6-carboxamide ditrifluoroacetate (Intermediate 41, 100 mg, 0.191 mmol) in DMSO (4 mL) was added 2-(2,6-dioxopiperidin-3-yl)-4-fluoroisoindole-1,3-dione (CAS No: 835616-60-9, 79.3 mg, 0.286 mmol) and DIPEA (148 mg, 1.15 mmol) and the reaction mixture was stirred at 120° C. for overnight. Water (5 mL) was added the resulting crude was purified by preparative HPLC (Column: WelFlash C18, Regular C18 20-40 m, 120 g; Mobile Phase A: Water (with 10 mmol/L NH₄HCO₃+0.05% NH₃·H₂O), Mobile Phase B: MeCN; Flow rate: 60 mL/min; Gradient: 35% B to 65% B in 30 min) to afford 2-(8-((2-(2,6-dioxopiperidin-3-yl)-1,3-dioxoisoindolin-4-yl)amino)octyl)-3-methyl-N-(2-((R)-1-methylpyrrolidin-2-yl)-1H-pyrrolo[3,2-c]pyridin-6-yl)-2H-indazole-6-carboxamide (Example 5, 15.2 mg, 10%) as a yellow solid. ¹H NMR (400 MH, DMSO-d6) δ ppm 11.37 (s, 1H), 11.10 (s, 1H), 10.43 (s, 1H), 8.51 (s, 1H), 8.29 (s, 1H), 8.20 (s, 1H), 7.75 (d, J=8.8 Hz, 1H), 7.58 (t, J=8.0 Hz, 2H), 7.09 (d, J=8.8 Hz, 1H), 7.02 (d, J=6.8 Hz, 1H), 6.52 (t, J=5.2 Hz, 1H), 6.40 (s, 1H), 5.03-5.07 (m, 1H), 4.39 (t, J=7.2 Hz, 2H), 3.24-3.32 (m, 2H), 3.15 (t, J=7.6 Hz, 1H), 2.87-2.93 (m, 1H), 2.83-2.84 (m, 3H), 2.57-2.65 (m, 1H), 2.26-2.34 (m, 1H), 2.12-2.17 (m, 4H), 1.99-2.11 (m, 2H), 1.78-1.97 (m, 6H), 1.51-1.59 (m, 2H), 1.29-1.39 (m, 8H). Mass spec: m/z: 758.5 [M+H]⁺.

### Example 6 were Synthesized Following Scheme 10

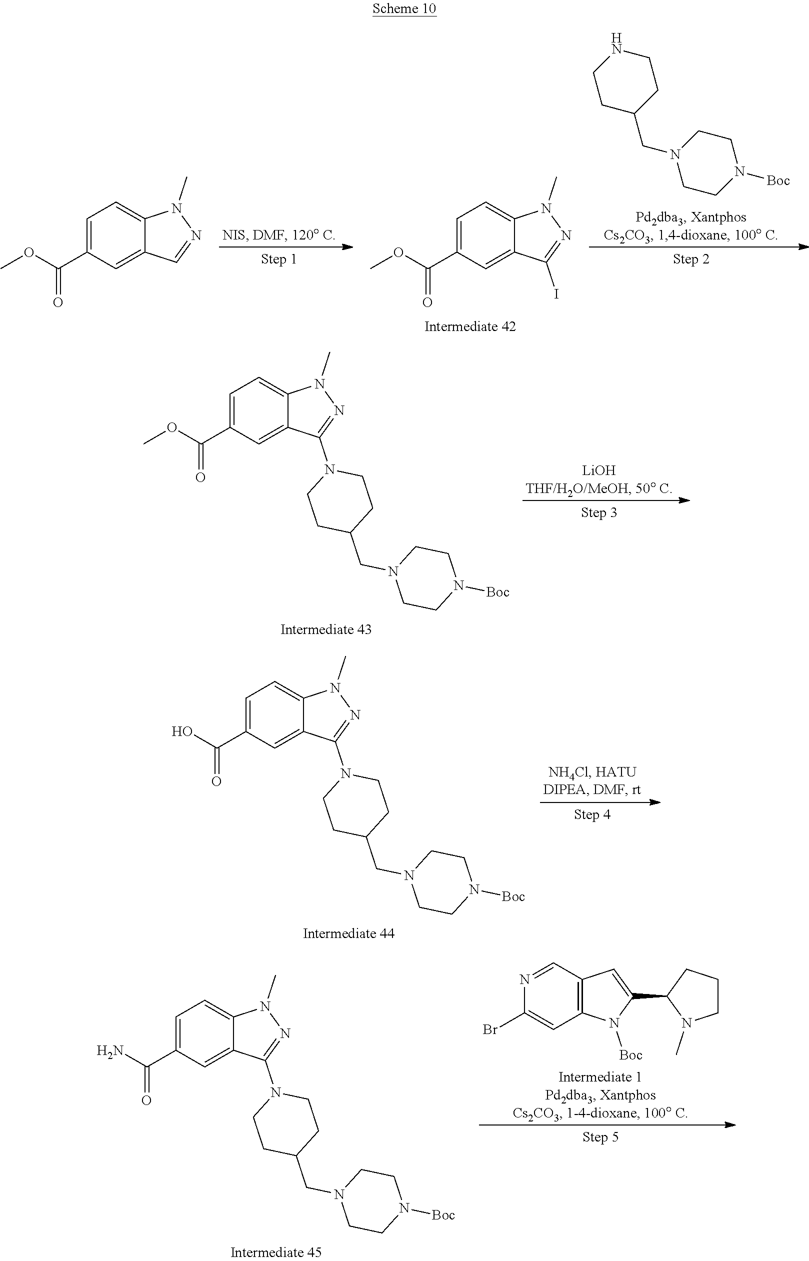

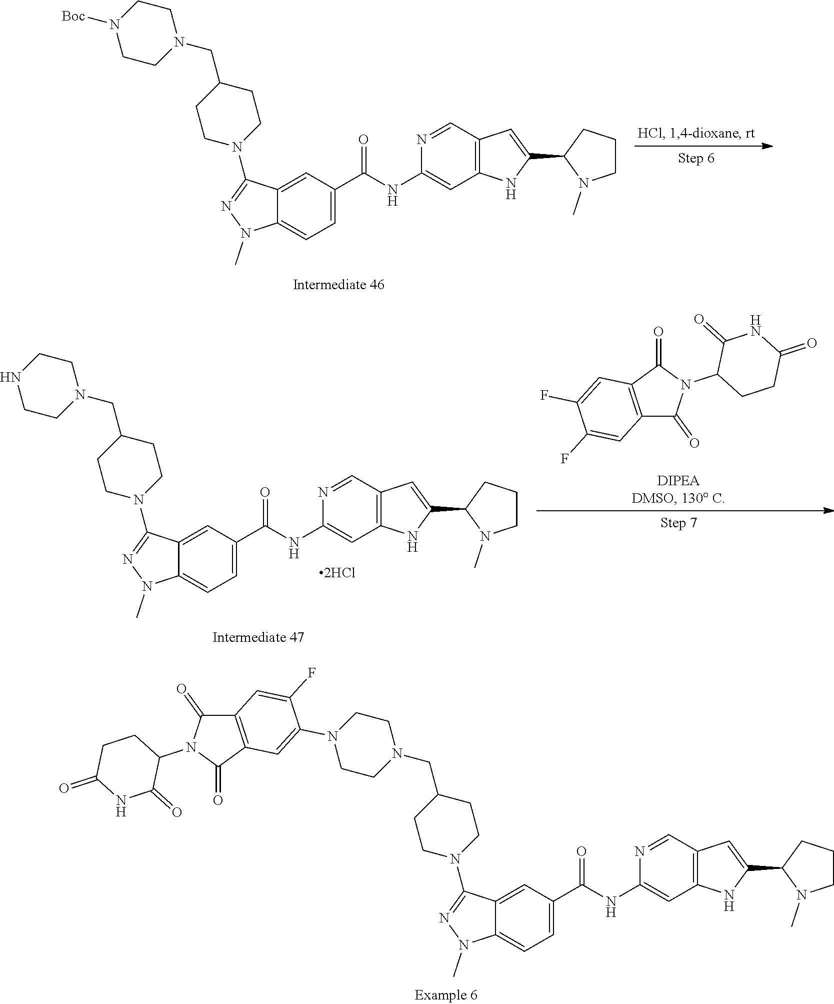 [SMILES: CN1CCC[C@@H]1c1cc2cnc(NC(=O)c3ccc4c(c3)c(N3CCC(CN5CCN(C(=O)OC(C)(C)C)CC5)CC3)nn4C)cc2[nH]1 ; CN1CCC[C@@H]1c1cc2cnc(NC(=O)c3ccc4c(c3)c(N3CCC(CN5CCNCC5)CC3)nn4C)cc2[nH]1 ; O=C1CCC(N2C(=O)c3cc(F)c(F)cc3C2=O)C(=O)N1 ; CN1CCC[C@@H]1c1cc2cnc(NC(=O)c3ccc4c(c3)c(N3CCC(CN5CCN(c6cc7c(cc6F)C(=O)N(C6CCC(=O)NC6=O)C7=O)CC5)CC3)nn4C)cc2[nH]1]

### Step 1

### Intermediate 42: methyl 3-iodo-1-methylindazole-5-carboxylate

To a solution of methyl 1-methylindazole-5-carboxylate (CAS: 1092351-82-0, 5.00 g, 25.5 mmol) in DMF (80 mL) was added NIS (13.2 g, 58.6 mmol). The reaction mixture was stirred at 120° C. overnight and then quenched with water. The mixture was extracted three times with EtOAc, the combined organic layers were washed with brine, dried over anhydrous Na₂SO₄, filtered, and concentrated under reduced pressure. The crude material was purified by flash chromatography on silica gel using petroleum ether/EtOAc (8/2) as eluent to afford methyl 3-iodo-1-methylindazole-5-carboxylate (Intermediate 42, 5 g, 25%) as a yellow solid. Mass spec: m/z: 317 [M+H]⁺.

### Step 2

### Intermediate 43: methyl 3-(4-{[4-(tert-butoxycarbonyl)piperazin-1-yl]methyl}piperidin-1-yl)-1-methylindazole-5-carboxylate

To a solution of methyl 3-iodo-1-methylindazole-5-carboxylate (Intermediate 42, 5.00 g, 6.33 mmol) and tert-butyl 4-(piperidin-4-ylmethyl)piperazine-1-carboxylate (CAS: 1211568-27-2, 1.80 g, 6.33 mmol) in 1,4-dioxane (80 mL) were added Xantphos (0.730 g, 1.26 mmol), Cs₂CO₃ (6.18 g, 19.0 mmol) and Pd₂dba₃ (0.580 g, 0.633 mmol). The reaction mixture was stirred at 100° C. overnight under nitrogen atmosphere. The reaction mixture was quenched with water and extracted three times with EtOAc. The combined organic extracts were washed with brine, dried over anhydrous Na₂SO₄, filtered, and concentrated under vacuum. The crude material was purified by flash chromatography on silica gel using petroleum ether/EtOAc (1/2) as eluent to afford methyl 3-(4-{[4-(tert-butoxycarbonyl)piperazin-1-yl]methyl}piperidin-1-yl)-1-methylindazole-5-carboxylate (Intermediate 43, 1.70 g, 44%) as a yellow solid. Mass spec: m/z: 472 [M+H]⁺.

### Step 3

### Intermediate 44: 3-(4-{[4-(tert-butoxycarbonyl)piperazin-1-yl]methyl}piperidin-1-yl)-1-methylindazole-5-carboxylic acid

To a solution of methyl 3-(4-{[4-(tert-butoxycarbonyl)piperazin-1-yl]methyl}piperidin-1-yl)-1-methylindazole-5-carboxylate (Intermediate 43, 1.70 g, 2.81 mmol) in THF (30 mL)/MeOH (10 mL)/H₂O (8 mL) was added LiOH (0.67 g, 28.1 mmol). The reaction mixture was stirred at 50° C. for 3 h. The reaction mixture was acidified to pH=5 with an aqueous solution HCl 4M, then concentrated under reduced pressure. The crude material was chromatographed by reverse-phase flash chromatography (C18) using MeCN/H₂O (72/28) as eluent to afford 3-(4-{[4-(tert-butoxycarbonyl)piperazin-1-yl]methyl}piperidin-1-yl)-1-methylindazole-5-carboxylic acid (Intermediate 44, 1.10 g, 77%) as a yellow solid. Mass spec: m/z: 458 [M+H]⁺.

### Step 4

### Intermediate 45: tert-butyl 4-{[1-(5-carbamoyl-1-methylindazol-3-yl)piperidin-4-yl]methyl}piperazine-1-carboxylate

A solution of 3-(4-{[4-(tert-butoxycarbonyl)piperazin-1-yl]methyl}piperidin-1-yl)-1-methylindazole-5-carboxylic acid (Intermediate 44, 1.00 g, 1.97 mmol), DIPEA (1.27 g, 9.83 mmol) and HATU (0.820 g, 2.16 mmol) in DMF (15 mL) was stirred at rt for 1 h. Then a solution of NH₄Cl (0.320 g, 5.90 mmol) in H₂O (1 mL) was added dropwise and the mixture was stirred for another 1 h. The reaction mixture was quenched with water and concentrated under reduced pressure. The crude material was chromatographed by reverse-phase flash chromatography (C18) using MeCN/H₂O (75/25) as eluent to afford tert-butyl 4-{[1-(5-carbamoyl-1-methylindazol-3-yl)piperidin-4-yl]methyl}piperazine-1-carboxylate (Intermediate 45, 900 mg, 98%) as a yellow solid. Mass spec: m/z: 457 [M+H]⁺.

### Step 5

### Intermediate 46: tert-butyl 4-({1-[1-methyl-5-({2-[(2R)-1-methylpyrrolidin-2-yl]-1H-pyrrolo[3,2-c]pyridin-6-yl}carbamoyl)indazol-3-yl]piperidin-4-yl}methyl)piperazine-1-carboxylate

To a solution of tert-butyl 4-{[1-(5-carbamoyl-1-methylindazol-3-yl)piperidin-4-yl]methyl}piperazine-1-carboxylate (Intermediate 45, 200 mg, 0.430 mmol) and tert-butyl 6-bromo-2-[(2R)-1-methylpyrrolidin-2-yl]pyrrolo[3,2-c]pyridine-1-carboxylate (Intermediate 1, 212 mg, 0.558 mmol) in 1,4-dioxane (6 mL) were added Cs₂CO₃ (420 mg, 1.29 mmol), Xantphos (99.3 mg, 0.172 mmol) and Pd₂dba₃ (39.3 mg, 0.043 mmol). The reaction mixture was stirred at 100° C. overnight under nitrogen atmosphere, then concentrated under reduced pressure. The residue was chromatographed by reverse-phase flash chromatography (C18) using MeCN/H₂O (28/72) as eluent to afford tert-butyl 4-({1-[1-methyl-5-({2-[(2R)-1-methylpyrrolidin-2-yl]-1H-pyrrolo[3,2-c]pyridin-6-yl}carbamoyl)indazol-3-yl]piperidin-4-yl}methyl)piperazine-1-carboxylate (Intermediate 46: 180 mg, 56%) as a yellow solid. Mass spec: m/z: 656 [M+H]⁺.

### Step 6

### Intermediate 47: 1-methyl-N-{2-[(2R)-1-methylpyrrolidin-2-yl]-1H-pyrrolo[3,2-c]pyridin-6-yl}-3-[4-(piperazin-1-ylmethyl)piperidin-1-yl]indazole-5-carboxamide dihydrochloride

To a stirred solution of tert-butyl 4-{[1-(5-{[1-(tert-butoxycarbonyl)-2-[(2R)-1-methylpyrrolidin-2-yl]pyrrolo[3,2-c]pyridin-6-yl]carbamoyl}-1-methylindazol-3-yl)piperidin-4-yl]methyl}piperazine-1-carboxylate (Intermediate 46, 180 mg, 0.207 mmol) in 1,4-dioxane (5 mL) was added a solution of HCl (2 mL, 12N in H₂O). The reaction mixture was stirred at rt for 2 h, then concentrated under reduced pressure to afford 1-methyl-N-{2-[(2R)-1-methylpyrrolidin-2-yl]-1H-pyrrolo[3,2-c]pyridin-6-yl}-3-[4-(piperazin-1-ylmethyl)piperidin-1-yl]indazole-5-carboxamide dihydrochloride (120 mg, 95%) as a yellow solid. Mass spec: m/z: 556 [M+H]⁺.

### Step 7

### Example 6: 3-[4-({4-[2-(2,6-dioxopiperidin-3-yl)-6-fluoro-1,3-dioxoisoindol-5-yl]piperazin-1-yl}methyl)piperidin-1-yl]-1-methyl-N-{2-[(2R)-1-methylpyrrolidin-2-yl]-1H-pyrrolo[3,2-c]pyridin-6-yl}indazole-5-carboxamide

A solution of 1-methyl-N-{2-[(2R)-1-methylpyrrolidin-2-yl]-1H-pyrrolo[3,2-c]pyridin-6-yl}-3-[4-(piperazin-1-ylmethyl)piperidin-1-yl]indazole-5-carboxamide dihydrochloride (Intermediate 47, 80.0 mg, 0.131 mmol) in DMSO (3 mL) were added 2-(2,6-dioxopiperidin-3-yl)-5,6-difluoroisoindole-1,3-dione (CAS: 1496997-41-1, 46.2 mg, 0.157 mmol) and DIPEA (169 mg, 1.31 mmol). The reaction mixture was stirred at 130° C. for 2h, quenched with water and concentrated under reduced pressure. The residue was purified by preparative HPLC (Column: XBridge OBD, 30×150 mm, 5 μm; Eluent: Water (10 mmol/L NH₄HCO₃+0.05% NH₃H₂O)/MeCN from 72/28 to 42/58; Flow rate: 60 mL/min) to afford 3-[4-({4-[2-(2,6-dioxopiperidin-3-yl)-6-fluoro-1,3-dioxoisoindol-5-yl]piperazin-1-yl}methyl)piperidin-1-yl]-1-methyl-N-{2-[(2R)-1-methylpyrrolidin-2-yl]-1H-pyrrolo[3,2-c]pyridin-6-yl}indazole-5-carboxamide (Example 6, 20.5 mg, 18%) as a yellow solid. Mass spec: m/z: 830 [M+H]; ¹H NMR (400 MHz, DMSO-d₆) δ ppm 11.36 (s, 1H), 11.10 (s, 1H), 10.59 (s, 1H), 8.52 (d, J=6.0 Hz, 2H), 8.20 (s, 1H), 8.02 (d, J=9.6 Hz, 1H), 7.72-7.75 (m, 1H), 7.45-7.52 (m, 2H), 6.39 (s, 1H), 5.08-5.13 (m, 1H), 4.00-4.03 (m, 2H), 3.87 (s, 3H), 3.27-3.31 (m, 5H), 3.14-3.16 (m, 1H), 2.84-2.87 (m, 3H), 2.67 (s, 1H), 2.50-2.57 (m, 5H), 2.30-2.32 (m, 3H), 2.14-2.28 (m, 4H), 2.02-2.04 (m, 1H), 1.90-2.04 (m, 4H), 1.81-1.88 (m, 2H), 1.32-1.46 (m, 2H).

### Example 7 was Synthesized Following Scheme 11

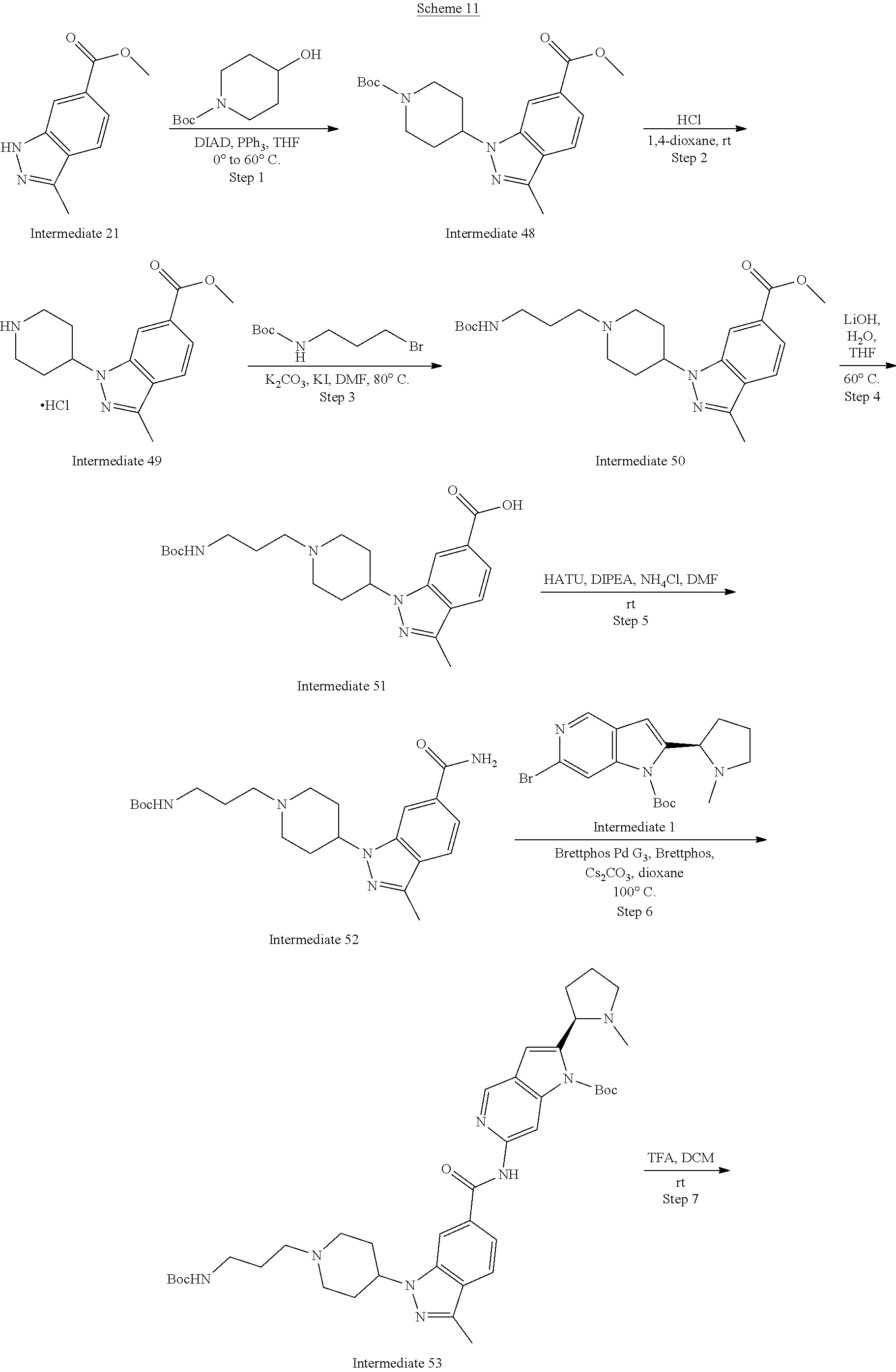

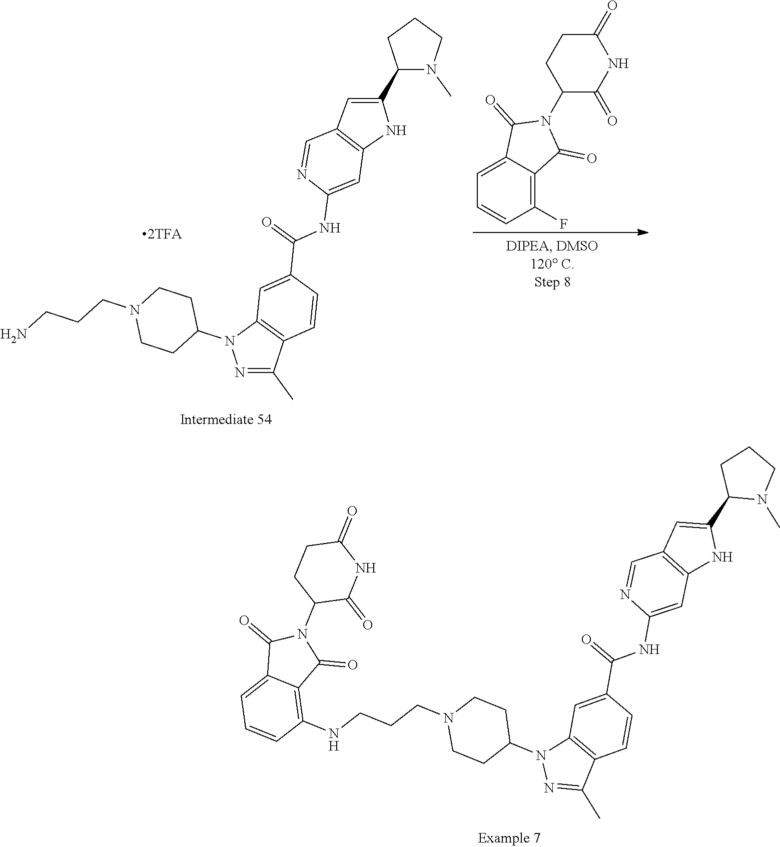

### Step 1

### Intermediate 48: methyl-1-(1-(tert-butoxycarbonyl)piperidin-4-yl)-3-methyl-1H-indazole-6-carboxylate

To a cooled (0° C.) solution of methyl 3-methyl-1H-indazole-6-carboxylate (Intermediate 21, 400 mg, 2.10 mmol), PPh₃ (1.10 g, 4.21 mmol) and tert-butyl 4-hydroxypiperidine-1-carboxylate (CAS No: 109384-19-2, 846 mg, 4.21 mmol) in tetrahydrofuran (10 mL) was added DIAD (850 mg, 4.21 mmol) dropwise and the resulting mixture was stirred overnight at 60° C. Water (300 mL) was then added and the mixture extracted with ethyl acetate (3×300 mL). The combined organic layers were washed with brine, dried over anhydrous Na₂SO₄, filtered and concentrated in vacuo. The crude material was purified by combi-flash column chromatography, by eluting with 14% ethyl acetate in heptane, then further purified by reverse-phase C18 combi-flash column chromatography, by eluting with 78% acetonitrile in water, to afford methyl 1-(1-(tert-butoxycarbonyl)piperidin-4-yl)-3-methyl-1H-indazole-6-carboxylate (Intermediate 48, 290 mg, 35%) as a colorless oil. Mass spec: m/z: 374.0 [M+H]⁺.

### Step 2

### Intermediate 49: methyl-3-methyl-1-(piperidin-4-yl)-1H-indazole-6-carboxylate hydrochloride

A solution of methyl-1-[1-(tert-butoxycarbonyl)piperidin-4-yl]-3-methylindazole-6-carboxylate (Intermediate 48, 290 mg, 0.787 mmol) in HCl (4M in 1,4-dioxane) was stirred at room temperature for 2 h. The resulting mixture was concentrated in vacuo to afford methyl 3-methyl-1-(piperidin-4-yl)-1H-indazole-6-carboxylate hydrochloride (Intermediate 49, 200 mg) as a colorless oil, which was used in the next step without further purification. Mass spec: m/z: 274.0 [M+H]⁺.

### Step 3

### Intermediate 50: methyl-1-(1-{3-[(tert-butoxycarbonyl)amino]propyl}piperidin-4-yl)-3-methylindazole-6-carboxylate

To a solution of methyl-3-methyl-1-(piperidin-4-yl)indazole-6-carboxylate hydrochloride (Intermediate 49, 200 mg, 0.732 mmol) and tert-butyl N-(3-bromopropyl)carbamate (CAS No: 83948-53-2, 261 mg, 1.10 mmol) in dimethylformamide (6 mL) was added potassium carbonate (303 mg, 2.19 mmol) and potassium iodide (24.3 mg, 0.146 mmol) and the reaction mixture was stirred at 80° C. for 2h. Water (200 mL) was added and the aqueous phase extracted with ethyl acetate (3×200 mL). The combined organic layers were washed with brine, dried over anhydrous Na₂SO₄, filtered, and concentrated in vacuo. The crude material was purified by combi-flash column chromatography, by eluting with 4% methanol in dichloromethane, to afford methyl-1-(1-{3-[(tert-butoxycarbonyl)amino]propyl}piperidin-4-yl)-3-methylindazole-6-carboxylate (Intermediate 50, 170 mg, 48%) as a colorless oil. Mass spec: m/z: 431.0 [M+H]⁺.

### Step 4

### Intermediate 51: 1-(1-(3-((tert-butoxycarbonyl)amino)propyl)piperidin-4-yl)-3-methyl-1H-indazole-6-carboxylic acid

To a solution of methyl-1-(1-{3-[(tert-butoxycarbonyl)amino]propyl}piperidin-4-yl)-3-methylindazole-6-carboxylate (Intermediate 50, 170 mg, 0.395 mmol) in THF (4 mL) and H₂O (1 mL) was added LiOH (47.4 mg, 1.97 mmol) and the reaction mixture was stirred at 60° C. for 2 h. The pH of the solution was adjusted to pH 5 by the dropwise addition of dilute aqueous HCl (1M) and the resulting mixture was extracted with CH₃Cl/i-PrOH (3:1, 3×200 mL). The combined organic layers were washed with brine, dried over anhydrous Na₂SO₄, filtered and concentrated in vacuo to afford 1-(1-(3-((tert-butoxycarbonyl)amino)propyl)piperidin-4-yl)-3-methyl-1H-indazole-6-carboxylic acid (Intermediate 51, 150 mg, 88%) as a brown solid, which was used in the next step without further purification. Mass spec: m/z: 417.0 [M+H]⁺.

### Step 5

### Intermediate 52: tert-butyl-(3-(4-(6-carbamoyl-3-methyl-1H-indazol-1-yl)piperidin-1-yl)propyl)carbamate

To a solution of 1-(1-{3-[(tert-butoxycarbonyl)amino]propyl}piperidin-4-yl)-3-methylindazole-6-carboxylic acid (Intermediate 51, 150 mg, 0.360 mmol) in DMF (4 mL) was added NH₄Cl (57.8 mg, 1.08 mmol), HATU (205 mg, 0.540 mmol) and DIPEA (233 mg, 1.80 mmol) and the reaction mixture was stirred at room temperature for 2 h. The reaction mixture was purified directly by reverse-phase C18 combi-flash column chromatography, by eluting with 70% acetonitrile in water, to afford tert-butyl-(3-(4-(6-carbamoyl-3-methyl-1H-indazol-1-yl)piperidin-1-yl)propyl)carbamate (Intermediate 52, 80 mg, 52%) as a brown oil. Mass spec: m/z: 416.0 [M+H]⁺.

### Step 6

### Intermediate 53: tert-butyl-(R)-6-(1-(1-(3-((tert-butoxycarbonyl)amino)propyl)piperidin-4-yl)-3-methyl-1H-indazole-6-carboxamido)-2-(1-methylpyrrolidin-2-yl)-1H-pyrrolo[3,2-c]pyridine-1-carboxylate

To a solution of tert-butyl-N-{3-[4-(6-carbamoyl-3-methylindazol-1-yl)piperidin-1-yl]propyl}carbamate (Intermediate 52, 80 mg, 0.193 mmol) and tert-butyl-6-bromo-2-[(2R)-1-methylpyrrolidin-2-yl]pyrrolo[3,2-c]pyridine-1-carboxylate (Intermediate 1, 73.2 mg, 0.193 mmol) in 1,4-dioxane (3 mL) was added BrettPhos Pd G3 (52.4 mg, 0.058 mmol), BrettPhos (62.0 mg, 0.116 mmol) and Cs₂CO₃ (125 mg, 0.386 mmol) and the reaction mixture was stirred at 100° C. for 4 h. The reaction mixture was concentrated in vacuo and the resulting residue was purified by preparative TLC, by eluting with 10% methanol in dichloromethane, to afford tert-butyl-(R)-6-(1-(1-(3-((tert-butoxycarbonyl)amino)propyl)piperidin-4-yl)-3-methyl-1H-indazole-6-carboxamido)-2-(1-methylpyrrolidin-2-yl)-1H-pyrrolo[3,2-c]pyridine-1-carboxylate (Intermediate 53, 80 mg, 57%) as a yellow oil. Mass spec: m/z: 715.0 [M+H]⁺.

### Step 7

### Intermediate 54: 1-[1-(3-aminopropyl)piperidin-4-yl]-3-methyl-N-{2-[(2R)-1-methylpyrrolidin-2-yl]-1H-pyrrolo[3,2-c]pyridin-6-yl}indazole-6-carboxamide ditrifluoroacetate

To a solution of tert-butyl 6-[1-(1-{3-[(tert-butoxycarbonyl)amino]propyl}piperidin-4-yl)-3-methylindazole-6-amido]-2-[(2R)-1-methylpyrrolidin-2-yl]pyrrolo[3,2-c]pyridine-1-carboxylate (Intermediate 53, 75 mg, 0.105 mmol) in dichloromethane (5 mL) was added TFA (1 mL) and the reaction mixture was stirred at room temperature for 2 h. The resulting mixture was concentrated in vacuo to afford 1-[1-(3-aminopropyl)piperidin-4-yl]-3-methyl-N-{2-[(2R)-1-methylpyrrolidin-2-yl]-1H-pyrrolo[3,2-c]pyridin-6-yl}indazole-6-carboxamide ditrifluoroacetate (Intermediate 54, 55 mg, 67%) as a brown oil, which was used in the next step without further purification. Mass spec: m/z: 515.0 [M+H]⁺.

### Step 8

### Example 7: 1-[1-(3-{[2-(2,6-dioxopiperidin-3-yl)-1,3-dioxoisoindol-4-yl]amino}propyl)piperidin-4-yl]-3-methyl-N-{2-[(2R)-1-methylpyrrolidin-2-yl]-1H-pyrrolo[3,2-c]pyridin-6-yl}indazole-6-carboxamide

To a solution of 1-[1-(3-aminopropyl)piperidin-4-yl]-3-methyl-N-{2-[(2R)-1-methylpyrrolidin-2-yl]-1H-pyrrolo[3,2-c]pyridin-6-yl}indazole-6-carboxamide ditrifluoroacetate (Intermediate 54, 55 mg, 0.107 mmol) and 2-(2,6-dioxopiperidin-3-yl)-4-fluoroisoindole-1,3-dione (CAS No: 835616-60-9, 44.3 mg, 0.161 mmol) in DMSO (3 mL) was added DIPEA (82.9 mg, 0.642 mmol) and the reaction mixture was stirred at 120° C. for 2 h. The reaction mixture was then purified directly by preparative HPLC (Column: XBridge Prep OBD C18 Column, 30*150 mm, 5 μm; Mobile Phase A: Water (10 mmol/L NH₄HCO₃), Mobile Phase B: MeCN; Flow rate: 60 mL/min; Gradient: 35% B to 65% B in 10 min), to afford 1-[1-(3-{[2-(2,6-dioxopiperidin-3-yl)-1,3-dioxoisoindol-4-yl]amino}propyl)piperidin-4-yl]-3-methyl-N-{2-[(2R)-1-methylpyrrolidin-2-yl]-1H-pyrrolo[3,2-c]pyridin-6-yl}indazole-6-carboxamide (Example 7, 11.7 mg, 14%) as a yellow solid. ¹H NMR (400 MHz, MeOH-d4) δ ppm 8.54 (s, 1H), 8.24-8.28 (m, 2H), 7.84 (d, J=8.4 Hz, 1H), 7.74 (d, J=8.4 Hz, 1H), 7.59 (t, J=8.4 Hz, 1H), 7.14 (d, J=8.8 Hz, 1H), 7.06 (d, J=7.2 Hz, 1H), 6.54 (s, 1H), 5.05-5.09 (m, 1H), 4.60-4.79 (m, 4H), 3.47-3.49 (m, 3H), 3.17-3.25 (m, 3H), 2.85-2.87 (m, 1H), 2.76-2.77 (m, 2H), 2.74-2.75 (m, 1H), 2.68-2.72 (m, 5H), 2.32-2.60 (m, 9H), 2.05-2.21 (m, 5H), 1.89-1.99 (m, 3H). Mass spec: m/z: 771.5 [M+H]⁺.

### Example 8 was Synthesized Following Scheme 12

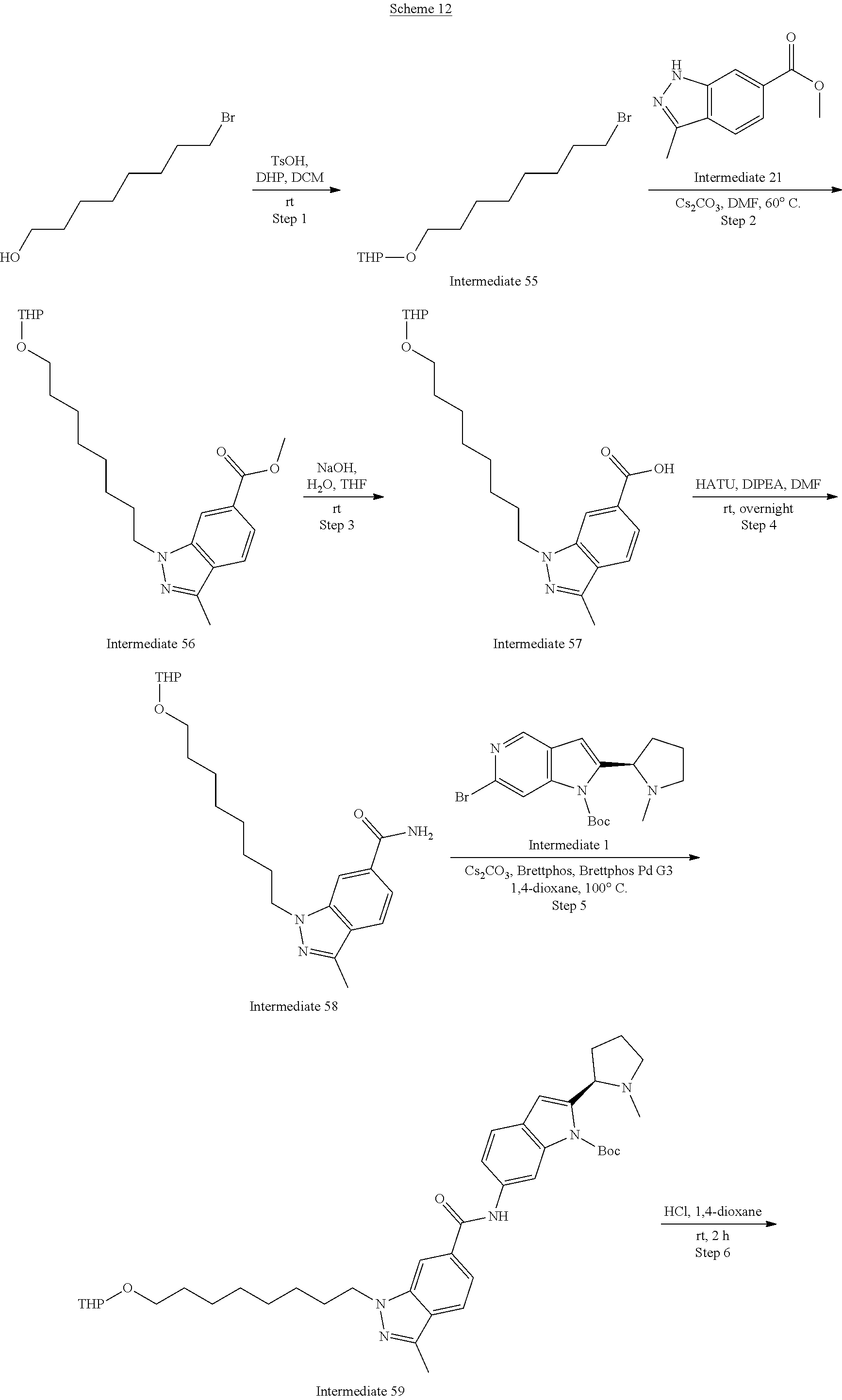

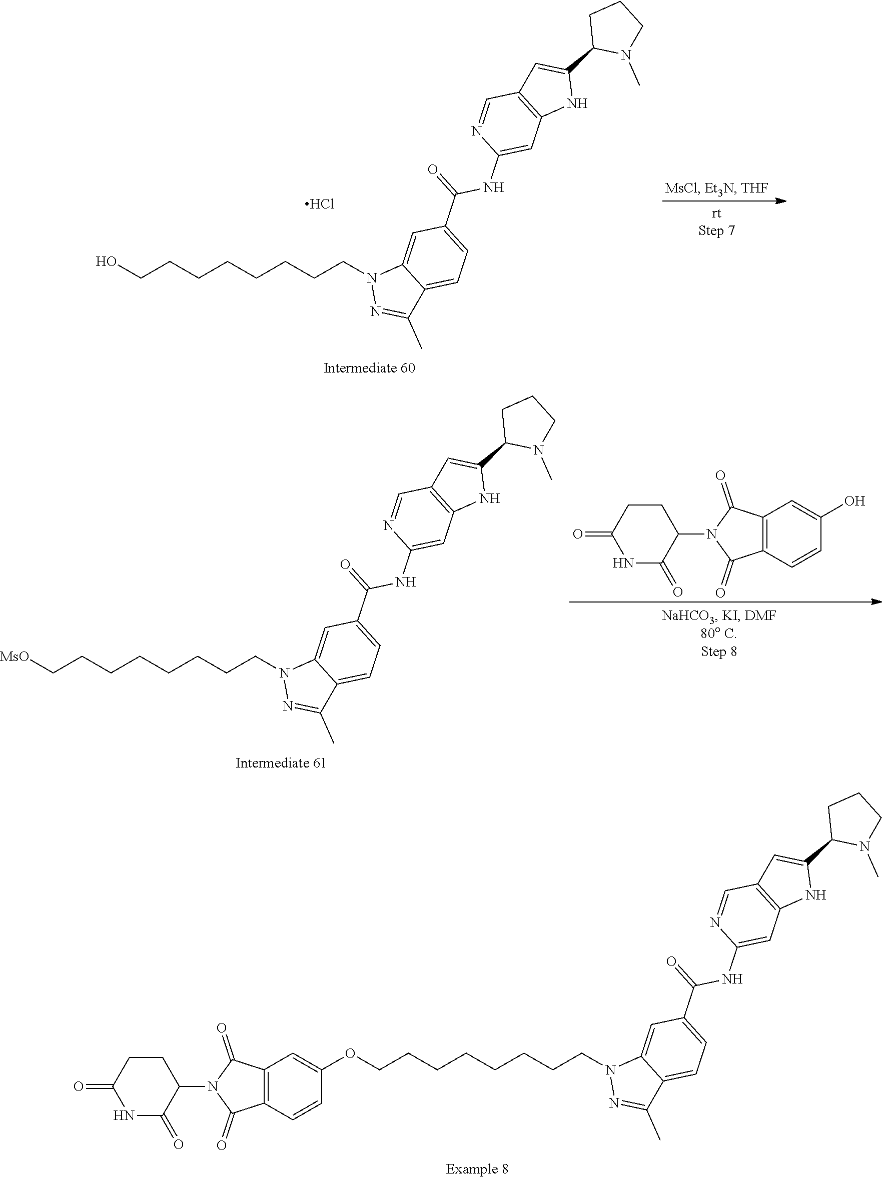 [SMILES: Cc1nn(CCCCCCCCO)c2cc(C(=O)Nc3cc4[nH]c([C@H]5CCCN5C)cc4cn3)ccc12 ; Cl ; Cc1nn(CCCCCCCCOS(C)(=O)=O)c2cc(C(=O)Nc3cc4[nH]c([C@H]5CCCN5C)cc4cn3)ccc12 ; O=C1CCC(N2C(=O)c3ccc(O)cc3C2=O)C(=O)N1 ; Cc1nn(CCCCCCCCOc2ccc3c(c2)C(=O)N(C2CCC(=O)NC2=O)C3=O)c2cc(C(=O)Nc3cc4[nH]c([C@H]5CCCN5C)cc4cn3)ccc12]

### Step 1

### Intermediate 55: 2-[(8-bromooctyl)oxy]oxane

To a solution of 8-bromooctan-1-ol (CAS No: 50816-19-8, 4.00 g, 19.1 mmol) and DHP (4.83 g, 57.3 mmol) in DCM (30.0 mL) was added TsOH (99.6 mg, 1.92 mmol) at 0° C. The reaction was stirred at room temperature overnight and quenched with water (300 ml). The resulting solution was extracted with EtOAc (3×100 mL) and the organic layers were combined, washed with brine (3×50 mL), dried over anhydrous sodium sulfate, filtered and concentrated under reduced pressure to afford 2-[(8-bromooctyl)oxy]oxane (Intermediate 55, 4.70 g, 80%) as a yellow oil. ¹H NMR (400 MHz, DMSO-d6) δ ppm 4.53-4.54 (m, 1H), 3.70-3.76 (m, 1H), 3.58-3.63 (m, 1H), 3.51-3.54 (m, 2H), 3.40-3.44 (m, 1H), 3.24-3.30 (m, 1H), 1.76-1.81 (m, 2H), 1.70-1.73 (m, 1H), 1.57-1.68 (m, 3H), 1.32-1.47 (m, 7H), 1.29 (br, 5H).

### Step 2

### Intermediate 56: methyl 3-methyl-1-[8-(oxan-2-yloxy)octyl]indazole-6-carboxylate

To a solution of methyl 3-methyl-1H-indazole-6-carboxylate (Intermediate 21, 1.00 g, 5.26 mmol) in DCM (10.0 mL) was added 2-[(8-bromooctyl)oxy]oxane (Intermediate 55, 4.63 g, 15.8 mmol) and Cs₂CO₃ (3.43 g, 10.5 mmol) and the reaction stirred at 60° C. overnight. Water (200 mL) was added and the resulting solution was extracted with EtOAc (3×300 mL) and the organic layers were combined, washed with brine (3×300 mL), dried over anhydrous sodium sulfate, filtered and concentrated under reduced pressure. The residue was chromatographed on a silica gel column, eluting with EtOAc/petroleum ether (11/89) to afford methyl 3-methyl-1-[8-(oxan-2-yloxy)octyl]indazole-6-carboxylate (Intermediate 56, 1.80 g, 54.4%) as an oil. Mass spec: m/z: 403.0 [M+H]⁺.

### Step 3

### Intermediate 57: 3-methyl-1-[8-(oxan-2-yloxy)octyl]indazole-6-carboxylic acid

To a solution of methyl 3-methyl-1-[8-(oxan-2-yloxy)octyl]indazole-6-carboxylate (Intermediate 56, 800 mg, 1.99 mmol) in THF (10.0 mL) was added 2M NaOH solution (5 mL, 10 mmol) and the reaction was stirred at room temperature for 2 h. The pH was adjusted to 6 by the addition of aqueous HCl (12M). The resulting mixture was extracted with EtOAc (3×100 mL) and the organic layers were combined, washed with brine (100 mL), dried over anhydrous sodium sulfate, filtered and concentrated under reduced pressure to afford 3-methyl-1-[8-(oxan-2-yloxy)octyl]indazole-6-carboxylic acid (Intermediate 57, 400 mg, 81.2%) as a yellow oil, which was used in the next step without further purification. Mass spec: m/z: 389.0 [M+H]⁺.

### Step 4

### Intermediate 58: 3-methyl-1-[8-(oxan-2-yloxy)octyl]indazole-6-carboxamide

To a solution of 3-methyl-1-[8-(oxan-2-yloxy)octyl]indazole-6-carboxylic acid (Intermediate 57, 400 mg, 1.03 mmol) in DMF (10.0 mL) was added DIPEA (798 mg, 6.18 mmol), HATU (391 mg, 1.03 mmol) and NH₄Cl (220 mg, 4.12 mmol) and the reaction was stirred overnight at room temperature. Water (10 mL) was added and the mixture was extracted with EtOAc (3×100 mL) and the organic layers were combined, washed with brine (100 mL), dried over anhydrous sodium sulfate, filtered and concentrated under reduced pressure. The residue was chromatographed on a silica gel column, eluting with ethyl acetate/petroleum ether (60/40), to afford 3-methyl-1-[8-(oxan-2-yloxy)octyl]indazole-6-carboxamide (Intermediate 58, 340 mg, 85%) as a white solid. m/z: 388.0 [M+H]⁺.

### Intermediate 59: tert-butyl-6-(3-methyl-1-(8-((tetrahydro-2H-pyran-2-yl)oxy)octyl)-1H-indazole-6-carboxamido)-2-((R)-1-methylpyrrolidin-2-yl)-1H-pyrrolo[3,2-c]pyridine-1-carboxylate

To a solution of 3-methyl-1-[8-(oxan-2-yloxy)octyl]indazole-6-carboxamide (Intermediate 58, 200 mg, 0.516 mmol) and tert-butyl-6-bromo-2-[(2R)-1-methylpyrrolidin-2-yl]pyrrolo[3,2-c]pyridine-1-carboxylate (Intermediate 1, 177 mg, 0.464 mmol) in 1,4-dioxane (10.0 mL) was added Brettphos (166 mg, 0.310 mmol), Brettphos Pd G3 (173 mg, 0.323 mmol) and Cs₂CO₃ (336 mg, 1.03 mmol) and the reaction was stirred overnight at 100° C. under nitrogen. Water (50 mL) was added, the mixture was extracted with EtOAc (3×100 mL) and the organic layers were combined, washed with brine (3×50 mL), dried over anhydrous sodium sulfate, filtered and concentrated under reduced pressure. The residue was chromatographed on a silica gel column, eluting with EtOAc/petroleum ether (30/70), to afford tert-butyl 6-(3-methyl-1-(8-((tetrahydro-2H-pyran-2-yl)oxy)octyl)-1H-indazole-6-carboxamido)-2-((R)-1-methylpyrrolidin-2-yl)-1H-pyrrolo[3,2-c]pyridine-1-carboxylate (Intermediate 59, 270 mg, 49%) as an off-white solid. Mass spec: m/z: 687.0 [M+H]⁺.

### Step 6

### Intermediate 60: 1-(8-hydroxyoctyl)-3-methyl-N-{2-[(2R)-1-methylpyrrolidin-2-yl]-1H-pyrrolo[3,2-c]pyridin-6-yl}indazole-6-carboxamide hydrochloride

To a solution of tert-butyl 6-{3-methyl-1-[8-(oxan-2-yloxy)octyl]indazole-6-amido}-2-[(2R)-1-methylpyrrolidin-2-yl]pyrrolo[3,2-c]pyridine-1-carboxylate (Intermediate 59, 270 mg, 0.393 mmol) in 1,4-dioxane (10.0 mL) was added conc. aqueous HCl (12M, 2.00 mL) and the reaction was stirred at room temperature for 2 h. The reaction was concentrated under reduced pressure to afford 1-(8-hydroxyoctyl)-3-methyl-N-{2-[(2R)-1-methylpyrrolidin-2-yl]-1H-pyrrolo[3,2-c]pyridin-6-yl}indazole-6-carboxamide hydrochloride (Intermediate 60, 120 mg) as an off-white solid, which was used in the next step without further purification. Mass spec: m/z: 503.0 [M+H]⁺.

### Step 7

### Intermediate 61: 8-[3-methyl-6-({2-[(2R)-1-methylpyrrolidin-2-yl]-1H-pyrrolo[3,2-c]pyridin-6-yl}carbamoyl)indazol-1-yl]octyl methanesulfonate

To a solution of 1-(8-hydroxyoctyl)-3-methyl-N-{2-[(2R)-1-methylpyrrolidin-2-yl]-1H-pyrrolo[3,2-c]pyridin-6-yl}indazole-6-carboxamide hydrochloride (Intermediate 60, 120 mg, 0.398 mmol) and Et₃N (313 mg, 1.91 mmol) in THF (10.0 mL) was added MsCl (82 mg, 0.716 mmol) at 0° C. and the reaction was stirred at room temperature for 2h. Water (20 mL) was added and the resulting mixture was extracted with EtOAc (3×100 mL) and the organic layers were combined, washed with brine (3×100 mL), dried over anhydrous sodium sulfate, filtered and concentrated under reduced pressure to afford 8-[3-methyl-6-({2-[(2R)-1-methylpyrrolidin-2-yl]-1H-pyrrolo[3,2-c]pyridin-6-yl}carbamoyl)indazol-1-yl]octyl methanesulfonate (Intermediate 61, 110 mg, 53%) as an off-white solid, which was used in the next step without further purification. Mass spec: m/z: 581.0 [M+H]⁺.

### Step 8

### Example 8: 1-(8-{[2-(2,6-dioxopiperidin-3-yl)-1,3-dioxoisoindol-5-yl]oxy}octyl)-3-methyl-N-{2-[(2R)-1-methylpyrrolidin-2-yl]-1H-pyrrolo[3,2-c]pyridin-6-yl}indazole-6-carboxamide

To a solution of 8-[3-methyl-6-({2-[(2R)-1-methylpyrrolidin-2-yl]-1H-pyrrolo[3,2-c]pyridin-6-yl}carbamoyl)indazol-1-yl]octyl methanesulfonate (Intermediate 61, 60.0 mg, 0.103 mmol) in DMF (3 mL) was added NaHCO₃ (17.4 mg, 0.206 mmol), KI (17.2 mg, 0.103 mmol) and 2-(2,6-dioxopiperidin-3-yl)-5-hydroxyisoindole-1,3-dione (CAS No: 64567-60-8, 28.3 mg, 0.103 mmol) and the reaction was stirred at 80° C. overnight. The crude reaction was then purified by preparative HPLC (Column: XBridge Prep OBD C18 Column, 30*150 mm, 5 m; Mobile Phase A: Water (with 10 mmol/L NH₄HCO₃), Mobile Phase B: MeCN; Flow rate: 60 mL/min; Gradient: 46% B to 76% B in 10 min) to afford 1-(8-{[2-(2,6-dioxopiperidin-3-yl)-1,3-dioxoisoindol-5-yl]oxy}octyl)-3-methyl-N-{2-[(2R)-1-methylpyrrolidin-2-yl]-1H-pyrrolo[3,2-c]pyridin-6-yl}indazole-6-carboxamide (Example 8, 4.3 mg, 8.5%) as a white solid. ¹H NMR (400 MHz, DMSO-d6) δ ppm 11.40 (s, 1H), 11.10 (s, 1H), 10.56 (s, 1H), 8.52 (s, 1H), 8.45 (s, 1H), 8.25 (s, 1H), 7.73-7.80 (m, 3H), 7.39 (s, 1H), 7.30 (d, J=8.4 Hz, 1H), 6.41 (s, 1H), 5.11 (t, J=12.8 Hz, 1H), 4.41 (s, 2H), 4.12 (s, 2H), 3.15-3.35 (m, 2H), 2.86-2.89 (m, 2H), 2.51-2.62 (m, 2H), 2.26-2.28 (m, 1H), 2.17 (s, 4H), 2.06-2.09 (m, 1H), 1.72-1.87 (m, 8H), 1.18-1.49 (m, 9H). Mass spec: m/z: 759.5 [M+H]⁺.

### Example 9 was Synthesized Following Scheme 13

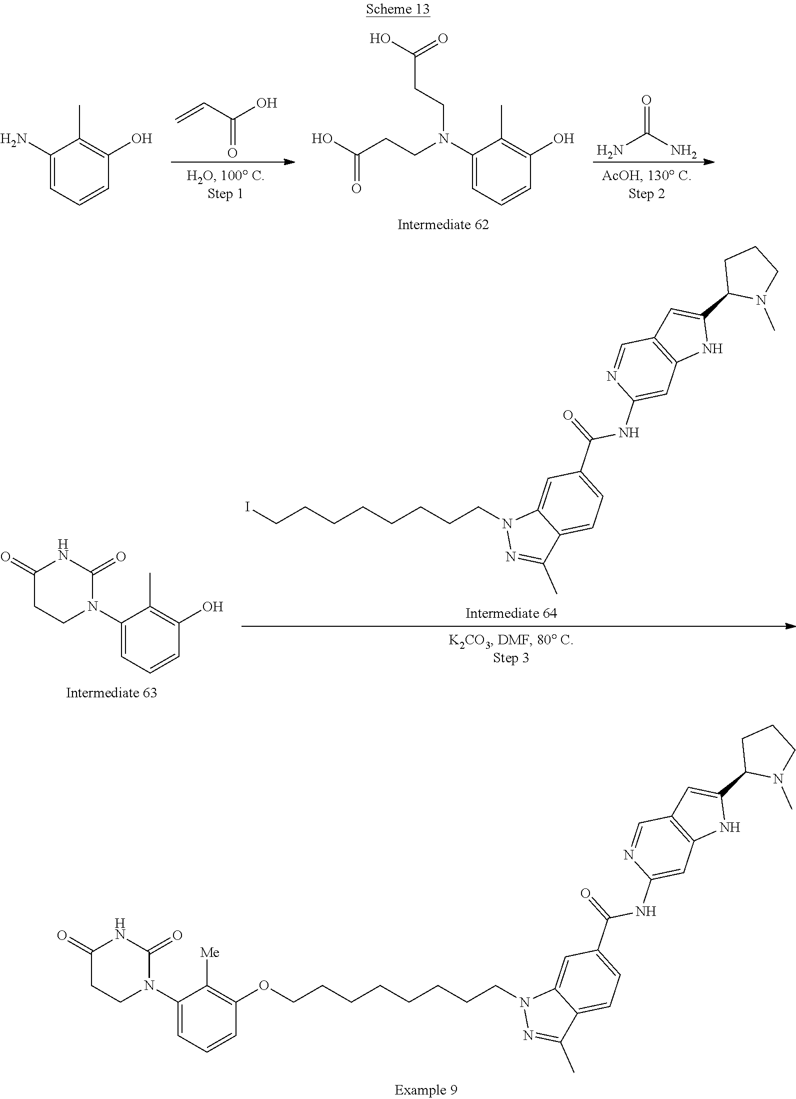

### Step 1

### Intermediate 62: 3-[(2-carboxyethyl)(3-hydroxy-2-methylphenyl)amino]propanoic acid

To a solution of 3-amino-2-methylphenol (CAS No: 53222-92-7, 2.00 g, 16.2 mmol) in H₂O (20.0 mL) was added acrylic acid (3.51 g, 48.7 mmol) and the reaction was stirred overnight at 100° C. The mixture was then chromatographed directly on a C18 silica column, eluting with water, to afford 3-[(2-carboxyethyl)(3-hydroxy-2-methylphenyl)amino]propanoic acid (Intermediate 62, 3.00 g, 58%) as a yellow oil. Mass spec: m/z: 268.0 [M+H]⁺.

### Step 2

### Intermediate 63: 1-(3-hydroxy-2-methylphenyl)-1,3-diazinane-2,4-dione

To a solution of 3-[(2-carboxyethyl)(3-hydroxy-2-methylphenyl)amino]propanoic acid (Intermediate 62, 1.70 g, 6.36 mmol) in acetic acid (12.8 mL) was added urea (573 mg, 9.54 mmol) and the reaction was stirred overnight at 130° C. The mixture was allowed to cool to room temperature, water (50 mL) was added, resulting in a formation of a precipitate. The precipitate was filtered, washed with 0.1% aqueous HCl (3×100 mL) and dried under vacuum to afford 1-(3-hydroxy-2-methylphenyl)-1,3-diazinane-2,4-dione (Intermediate 63, 500 mg, 42%) as an off-white solid. Mass spec: m/z: 221.0 [M+H]⁺.

### Step 3

### Example 9: 1-{8-[3-(2,4-dioxo-1,3-diazinan-1-yl)-2-methylphenoxy]octyl}-3-methyl-N-{2-[(2R)-1-methylpyrrolidin-2-yl]-1H-pyrrolo[3,2-c]pyridin-6-yl}indazole-6-carboxamide

To a solution of 1-(3-hydroxy-2-methylphenyl)-1,3-diazinane-2,4-dione (Intermediate 63, 27.7 mg, 0.125 mmol) and 1-(8-iodooctyl)-3-methyl-N-{2-[(2R)-1-methylpyrrolidin-2-yl]-1H-pyrrolo[3,2-c]pyridin-6-yl}indazole-6-carboxamide (Intermediate 64, 70.0 mg, 0.114 mmol) in DMF (3.50 mL) was added K₂CO₃ (63.2 mg, 0.456 mmol) and the reaction was stirred at 80° C. for 2 h. The resulting solution was purified directly by preparative HPLC (Column: XBridge Prep Phenyl OBD C18 Column, 30*150 mm, 5 μm; Mobile Phase A: Water (10 mmol/L NH₄HCO₃), Mobile Phase B: MeCN; Flow rate: 60 mL/min; Gradient: 40% B to 70% B in 10 min) to afford 1-{8-[3-(2,4-dioxo-1,3-diazinan-1-yl)-2-methylphenoxy]octyl}-3-methyl-N-{2-[(2R)-1-methylpyrrolidin-2-yl]-1H-pyrrolo[3,2-c]pyridin-6-yl}indazole-6-carboxamide (Example 9, 1.7 mg, 2%) as a white solid. ¹H NMR 6 ppm 11.42 (s, 1H), 10.54 (s, 1H), 10.32 (s, 1H), 8.52 (s, 1H), 8.44 (s, 1H), 8.25 (s, 1H), 7.72-7.80 (m, 2H), 7.14 (t, J=8.8 Hz, 1H), 6.82-6.87 (m, 2H), 6.41 (s, 1H), 4.41-4.45 (m, 3H), 3.93 (t, J=6.4 Hz, 2H), 3.70-3.76 (m, 2H), 3.15-3.16 (m, 2H), 2.18-2.33 (m, 4H), 1.96 (s, 4H), 1.82-1.88 (m, 5H), 1.71-1.72 (m, 3H), 1.32-1.36 (m, 3H), 1.24-1.26 (m, 7H), 1.19-1.21 (m, 1H). Mass spec: m/z: 705.5 [M+H]⁺.

### Example 10 was Synthesized Following Scheme 14

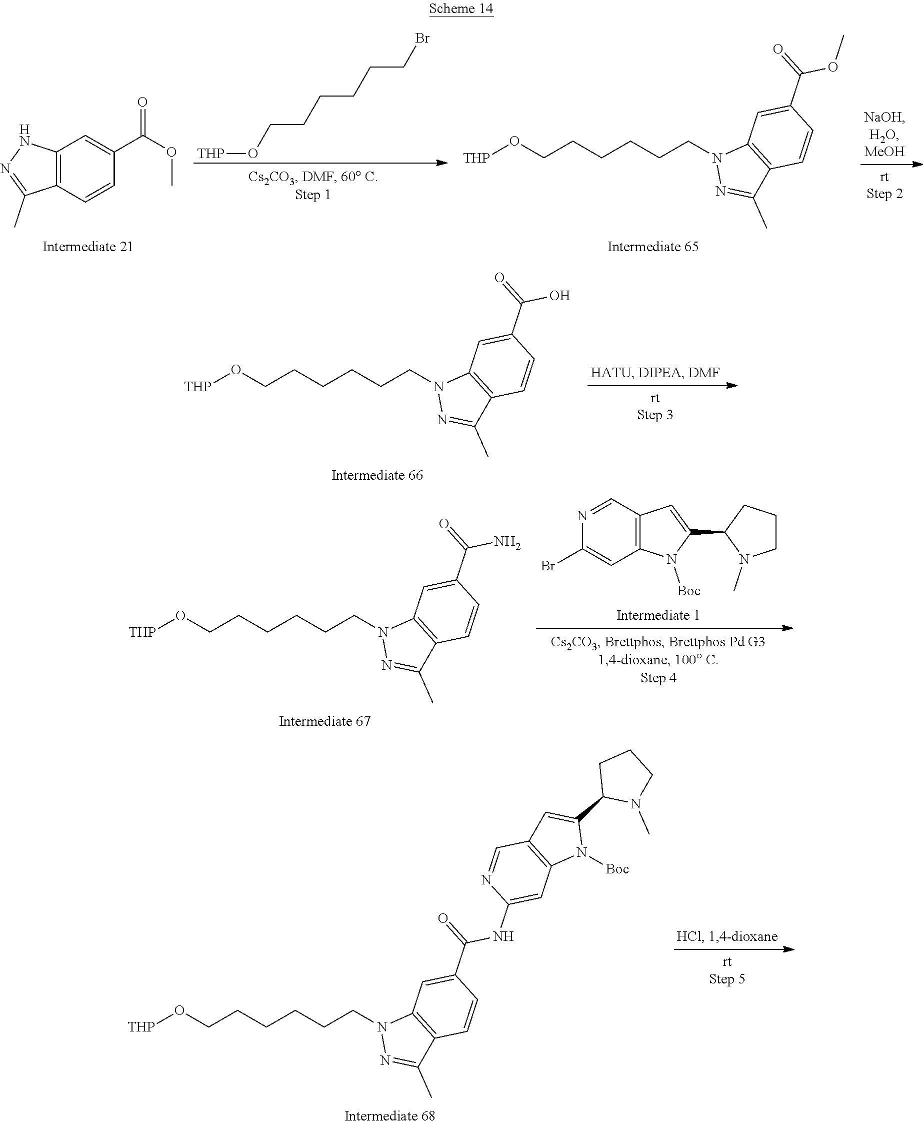

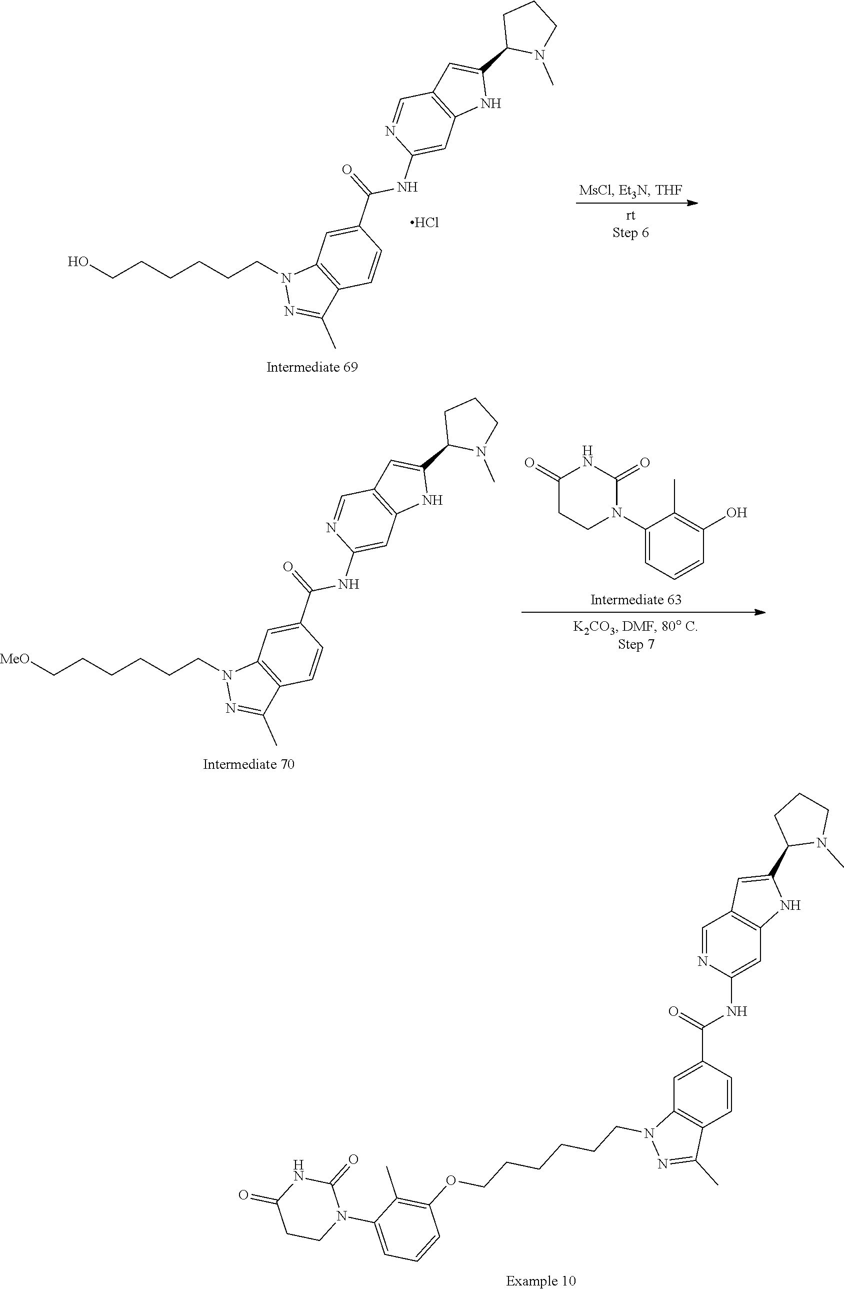

### Step 1

### Intermediate 65: methyl 3-methyl-1-[6-(oxan-2-yloxy)hexyl]indazole-6-carboxylate

To a solution of methyl 3-methyl-1H-indazole-6-carboxylate (Intermediate 21, 1.00 g, 5.26 mmol) in DMF (20.0 mL) was added 2-(6-Bromohexyloxy)tetrahydro-2H-pyran (CAS No: 53963-10-3, 2.09 g, 7.89 mmol) and Cs₂CO₃ (3.43 g, 10.5 mmol) and the reaction was stirred at 60° C. for 2h. Water (50 mL) was added and the aqueous phase was extracted with EtOAc (3×200 mL). The combined organic phases were washed with brine (3×200 mL), dried over anhydrous sodium sulfate, filtered and concentrated under reduced pressure. The resulting residue was chromatographed on a silica gel column, eluting with EtOAc/petroleum ether (20/80), to afford methyl 3-methyl-1-[6-(oxan-2-yloxy)hexyl]indazole-6-carboxylate (Intermediate 65, 1.30 g, 61%) as a yellow oil. Mass spec: m/z: 375.0 [M+H]⁺.

### Step 2

### Intermediate 66: 3-methyl-1-[6-(oxan-2-yloxy)hexyl]indazole-6-carboxylic acid

To a solution of methyl 3-methyl-1-[6-(oxan-2-yloxy)hexyl]indazole-6-carboxylate (Intermediate 65, 1.30 g, 3.47 mmol) in MeOH (25 mL) was added NaOH (4M in H₂O, 5 mL) and the reaction was stirred at room temperature for 2h. Water (50 mL) was added and the pH the solution was adjusted to pH 6 by dropwise addition of concentrated aqueous HCl. The mixture was then extracted with EtOAc (3×200 mL), washed with brine (3×200 mL), dried over anhydrous sodium sulfate and concentrated under reduced pressure to afford 3-methyl-1-[6-(oxan-2-yloxy)hexyl]indazole-6-carboxylic acid (Intermediate 66, 1.10 g, 87%) as a colourless oil. Mass spec: m/z: 361.0 [M+H]⁺.

### Step 3

### Intermediate 67: 3-methyl-1-[6-(oxan-2-yloxy)hexyl]indazole-6-carboxamide

To a solution of 3-methyl-1-[6-(oxan-2-yloxy)hexyl]indazole-6-carboxylic acid (Intermediate 66, 800 mg, 2.22 mmol) in DMF (10 mL) was added DIPEA (1.72 g, 13.3 mmol), HATU (844 mg, 2.22 mmol) and NH₄Cl (475 mg, 8.88 mmol) and the reaction was stirred at room temperature for 4h. Water (20 mL) was added and the aqueous phase extracted with EtOAc (3×100 mL). The combined organic phases were washed with brine (3×100 mL), dried over anhydrous sodium sulfate, filtered and concentrated under reduced pressure. The resulting residue was chromatographed on a silica gel column, eluting with EtOAc/petroleum ether (65/35) to afford 3-methyl-1-[6-(oxan-2-yloxy)hexyl]indazole-6-carboxamide (Intermediate 67, 700 mg, 99%) as a colorless oil. Mass spec: m/z: 360.0 [M+H]⁺.

### Step 4

### Intermediate 68: tert-butyl 6-{3-methyl-1-[6-(oxan-2-yloxy)hexyl]indazole-6-amido}-2-[(2R)-1-methylpyrrolidin-2-yl]pyrrolo[3,2-c]pyridine-1-carboxylate

To a solution of 3-methyl-1-[6-(oxan-2-yloxy)hexyl]indazole-6-carboxamide (Intermediate 67, 700 mg, 1.95 mmol) in 1,4-dioxane (10.0 ml) was added tert-butyl 6-bromo-2-[(2R)-1-methylpyrrolidin-2-yl]pyrrolo[3,2-c]pyridine-1-carboxylate (Intermediate 1, 0.665 g, 1.75 mmol), Brettphos (0.630 g, 1.168 mmol), Brettphos Pd G3 (0.532 g, 0.585 mmol) and Cs₂CO₃ (1.27 g, 3.89 mmol) and the reaction mixture was stirred at 100° C. for 4h, under N₂. The volatiles were then removed under reduced pressure and the resulting residue was purified on a C18 silica column, eluting with MeCN, to afford tert-butyl 6-{3-methyl-1-[6-(oxan-2-yloxy)hexyl]indazole-6-amido}-2-[(2R)-1-methylpyrrolidin-2-yl]pyrrolo[3,2-c]pyridine-1-carboxylate (Intermediate 68, 500 mg, 37%) as a yellow solid. Mass spec: m/z: 659.0 [M+H]⁺.

### Step 5

### Intermediate 69: 1-(6-hydroxyhexyl)-3-methyl-N-{2-[(2R)-1-methylpyrrolidin-2-yl]-1H-pyrrolo[3,2-c]pyridin-6-yl}indazole-6-carboxamide hydrochloride

To a solution of tert-butyl 6-{3-methyl-1-[6-(oxan-2-yloxy)hexyl]indazole-6-amido}-2-[(2R)-1-methylpyrrolidin-2-yl]pyrrolo[3,2-c]pyridine-1-carboxylate (Intermediate 68, 500 mg, 0.759 mmol) in 1,4-dioxane (16.6 mL) was added concentrated aqueous HCl (8.3 mL) and the reaction was stirred at room temperature for 2 h. The volatiles were then removed under reduced pressure to afford 1-(6-hydroxyhexyl)-3-methyl-N-{2-[(2R)-1-methylpyrrolidin-2-yl]-1H-pyrrolo[3,2-c]pyridin-6-yl}indazole-6-carboxamide hydrochloride (Intermediate 69, 330 mg, 97%) as a yellow oil. Mass spec: m/z: 475.0 [M+H]⁺.

### Step 6

### Intermediate 70: 6-[3-methyl-6-({2-[(2R)-1-methylpyrrolidin-2-yl]-1H-pyrrolo[3,2-c]pyridin-6-yl}carbamoyl)indazol-1-yl]hexyl methanesulfonate

To a solution of 1-(6-hydroxyhexyl)-3-methyl-N-{2-[(2R)-1-methylpyrrolidin-2-yl]-1H-pyrrolo[3,2-c]pyridin-6-yl}indazole-6-carboxamide hydrochloride (Intermediate 69, 150 mg, 0.316 mmol) and Et₃N (320 mg, 3.16 mmol) in THF (4.50 mL) was added MsCl (109 mg, 0.948 mmol), at 0° C., and the reaction was stirred at room temperature for 2h. Water (10 mL) was added and the resulting aqueous phase was extracted with EtOAc (3×50 mL). The organic layers were combined, washed with brine (3×50 mL), dried over anhydrous sodium sulfate, filtered and concentrated under reduced pressure to afford 6-[3-methyl-6-({2-[(2R)-1-methylpyrrolidin-2-yl]-1H-pyrrolo[3,2-c]pyridin-6-yl}carbamoyl)indazol-1-yl]hexyl methanesulfonate (Intermediate 70, 170 mg, 81%) as an off-white solid, which was used in the next step without further purification. Mass spec: m/z: 553.0 [M+H]⁺.

### Step 7

### Example 10: 1-{6-[3-(2,4-dioxo-1,3-diazinan-1-yl)-2-methylphenoxy]hexyl}-3-methyl-N-{2-[(2R)-1-methylpyrrolidin-2-yl]-1H-pyrrolo[3,2-c]pyridin-6-yl}indazole-6-carboxamide

To a solution of 1-(6-hydroxyhexyl)-3-methyl-N-{2-[(2R)-1-methylpyrrolidin-2-yl]-1H-pyrrolo[3,2-c]pyridin-6-yl}indazole-6-carboxamide (Intermediate 70, 100 mg, 0.211 mmol) in DMF (5.00 mL) was added 1-(3-hydroxy-2-methylphenyl)-1,3-diazinane-2,4-dione (Intermediate 63, 51.0 mg, 0.232 mmol) and K₂CO₃ (116 mg, 0.844 mmol) and the reaction was stirred at 80° C. for 2 h. The resulting mixture was purified directly by preparative HPLC (Column: Xselect CSH Prep C18 Column, 30*150 mm, 5 μm; Mobile Phase A: Water (with 0.1% formic acid), Mobile Phase B: MeCN; Flow rate: 60 mL/min; Gradient: 5% B to 35% B in 10 min) to afford 1-{6-[3-(2,4-dioxo-1,3-diazinan-1-yl)-2-methylphenoxy]hexyl}-3-methyl-N-{2-[(2R)-1-methylpyrrolidin-2-yl]-1H-pyrrolo[3,2-c]pyridin-6-yl}indazole-6-carboxamide (Example 10, 4.5 mg, 4.3%) as a white solid. ¹H NMR (400 MHz, DMSO-d6) δ ppm 11.43 (s, 1H), 10.54 (s, 1H), 10.31 (s, 1H), 8.53 (s, 1H), 8.45 (s, 1H), 8.25 (s, 1H), 7.73-7.80 (m, 2H), 7.15 (t, J=8.0 Hz, 1H), 6.83-6.89 (m, 2H), 6.41 (s, 1H), 4.43 (t, J=6.8 Hz, 2H), 3.95 (t, J=6.0 Hz, 2H), 3.68-3.71 (m, 3H), 3.13-3.16 (m, 2H), 2.71-2.75 (m, 3H), 2.27-2.34 (m, 2H), 2.15-2.18 (m, 3H), 1.93 (s, 3H), 1.72-1.91 (m, 7H), 1.49-1.52 (m, 2H), 1.37-1.41 (m, 2H), 0.94-0.96 (m, 1H). Mass spec: m/z: 677.50 [M+H]⁺.

### Example 11 was Synthesized Following Scheme 15

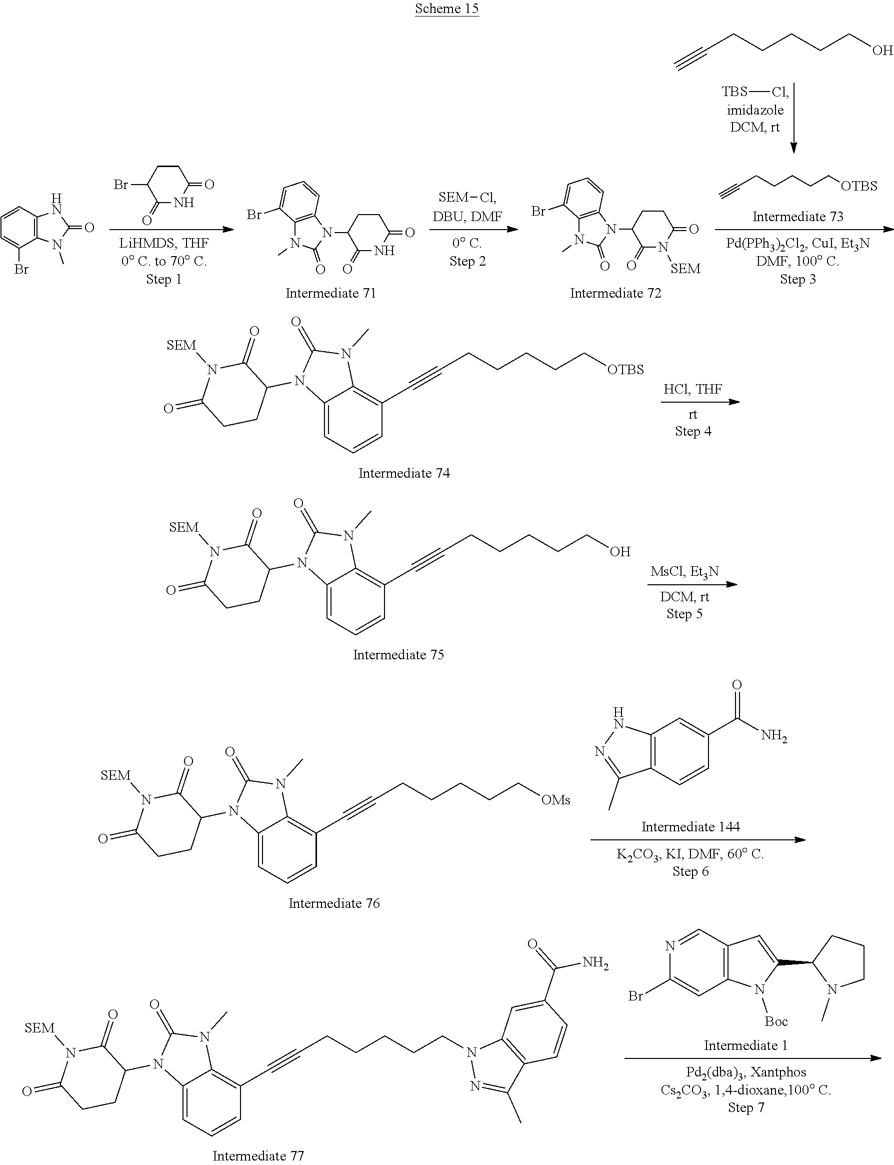

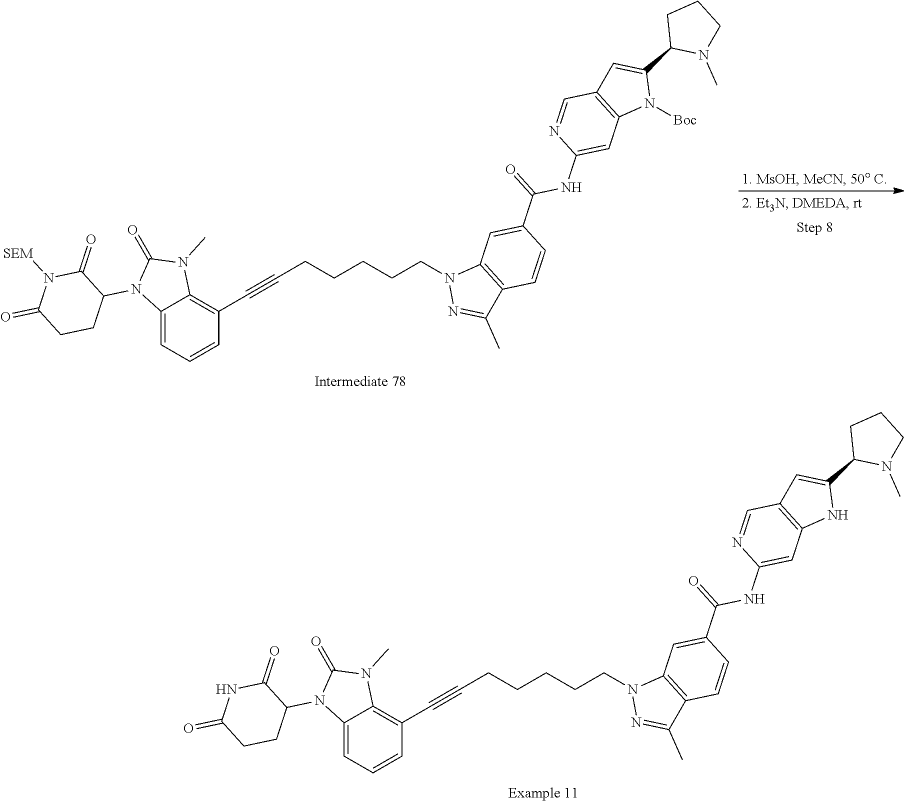 [SMILES: Cc1nn(CCCCCC#Cc2cccc3c2n(C)c(=O)n3C2CCC(=O)N(COCC[Si](C)(C)C)C2=O)c2cc(C(=O)Nc3cc4c(cn3)cc([C@H]3CCCN3C)n4C(=O)OC(C)(C)C)ccc12 ; Cc1nn(CCCCCC#Cc2cccc3c2n(C)c(=O)n3C2CCC(=O)NC2=O)c2cc(C(=O)Nc3cc4[nH]c([C@H]5CCCN5C)cc4cn3)ccc12]

### Step 1

### Intermediate 71: 3-(4-bromo-3-methyl-2-oxo-1,3-benzodiazol-1-yl)piperidine-2,6-dione

To a solution of 7-bromo-1-methyl-3H-1,3-benzodiazol-2-one (CAS No: 913297-44-6, 2.00 g, 8.72 mmol) in THF (20 mL) was added LiHMDS (26.2 mL, 26.2 mmol, 1M in THF) dropwise at 0° C. under N₂. After 15 min, 3-bromopiperidine-2,6-dione (CAS No: 62595-74-8, 2.51 g, 13.1 mmol) in THF (5 mL) was added dropwise at 0° C. and the reaction was stirred at 70° C. for 4 h under N₂. The reaction mixture was then cooled to 0° C. and pH of the mixture was adjusted to pH 2-3 with dilute aqueous HCl (1M) resulting in a precipitate. The precipitate was collected by filtration to afford 3-(4-bromo-3-methyl-2-oxo-1,3-benzodiazol-1-yl)piperidine-2,6-dione (Intermediate 71, 2.00 g, 34%) as an off white solid. Mass spec: m/z: 338.0 [M+H]⁺.

### Step 2

### Intermediate 72: 3-(4-bromo-3-methyl-2-oxo-1,3-benzodiazol-1-yl)-1-{[2-(trimethylsilyl)ethoxy]methyl}piperidine-2,6-dione

To a stirred solution of 3-(4-bromo-3-methyl-2-oxo-1,3-benzodiazol-1-yl)piperidine-2,6-dione (Intermediate 71, 1.9 g, 2.81 mmol) and DBU (0.86 g, 5.62 mmol) in DMF (40 mL) was added SEM-Cl (0.94 g, 5.62 mmol) at 0° C. The resulting mixture was allowed to warm to room temperature and stirred overnight. The mixture was quenched with water (100 mL) and extracted with EtOAc (3×200 mL). The combined organic layers were washed with brine, dried over anhydrous Na₂SO, filtered and concentrated under reduced pressure. The residue was chromatographed on a silica gel column, eluting with EtOAc/petroleum ether (35/65), to afford 3-(4-bromo-3-methyl-2-oxo-1,3-benzodiazol-1-yl)-1-{[2-(trimethylsilyl)ethoxy]methyl}piperidine-2,6-dione (Intermediate 72, 1.5 g, 91%) as a an oil. Mass spec: m/z: 468.1 [M+H]⁺.

### Step 3

### Intermediate 74: 3-(4-{7-[(tert-butyldimethylsilyl)oxy]hept-1-yn-1-yl}-3-methyl-2-oxo-1,3-benzodiazol-1-yl)-1-{[2-(trimethylsilyl)ethoxy]methyl}piperidine-2,6-dione

To a stirred solution of hept-6-yn-1-ol (CAS No: 63478-76-2, 500 mg, 4.413 mmol) in DCM (10 mL) was added imidazole (360 mg, 5.296 mmol) and TBS-Cl (798 mg, 5.296 mmol) and the reaction mixture was stirred overnight at room temperature. The resulting mixture was filtered and concentrated under reduced pressure to afford tert-butyl(hept-6-yn-1-yloxy)dimethylsilane (Intermediate 73, 1.00 g, 91%) as a colorless liquid, which was used in the stage without further purification. A 50 mL round-bottom flask was charged with 3-(4-bromo-3-methyl-2-oxo-1,3-benzodiazol-1-yl)-1-{[2-(trimethylsilyl)ethoxy]methyl}piperidine-2,6-dione (Intermediate 72, 700 mg, 1.196 mmol), tert-butyl(hept-6-yn-1-yloxy)dimethylsilane (541 mg, 2.39 mmol), Pd(PPh₃)₂Cl₂ (134 mg, 0.191 mmol), CuI (45.5 mg, 0.239 mmol), triethylamine (3 mL, 21.58 mmol) in DMF (10 mL) under N₂ and the reaction was stirred overnight at 100° C. The mixture was quenched with water (100 mL) and extracted with EtOAc (3×100 mL). The combined organic layers were washed with brine, dried over anhydrous Na₂SO₄, filtered, and concentrated under reduced pressure. The residue was chromatographed on a silica gel column, eluting with petroleum ether/EtOAc (38/72) to afford 3-(4-{7-[(tert-butyldimethylsilyl)oxy]hept-1-yn-1-yl}-3-methyl-2-oxo-1,3-benzodiazol-1-yl)-1-{[2-(trimethylsilyl)ethoxy]methyl}piperidine-2,6-dione (Intermediate 74, 900 mg, 86%) as a yellow oil. Mass spec: m/z: 614.0 [M+H]⁺.

### Step 4

### Intermediate 75: 3-[4-(7-hydroxyhept-1-yn-1-yl)-3-methyl-2-oxo-1,3-benzodiazol-1-yl]-1-{[2-(trimethylsilyl)ethoxy]methyl}piperidine-2,6-dione

To a stirred solution of 3-(4-{7-[(tert-butyldimethylsilyl)oxy]hept-1-yn-1-yl}-3-methyl-2-oxo-1,3-benzodiazol-1-yl)-1-{[2-(trimethylsilyl)ethoxy]methyl}piperidine-2,6-dione (Intermediate 74, 870 mg, 0.992 mmol) in THF (15 mL) was added HCl (4M in 1,4-dioxane, 3 mL) at room temperature and the reaction stirred for 0.5h. The mixture was quenched with water (100 mL) and extracted with EtOAc (3×100 mL). The combined organic layers were washed with brine, dried over anhydrous Na₂SO₄, filtered and concentrated under reduced pressure. The residue was chromatographed on a silica gel column, eluting with DCM/MeOH (96/4), to afford 3-[4-(7-hydroxyhept-1-yn-1-yl)-3-methyl-2-oxo-1,3-benzodiazol-1-yl]-1-{[2-(trimethylsilyl)ethoxy]methyl}piperidine-2,6-dione (Intermediate 75, 450 mg, 73%) as a yellow oil. Mass spec: m/z: 500.0 [M+H]⁺.

### Step 5

### Intermediate 76: 7-[1-(2,6-dioxo-1-{[2-(trimethylsilyl)ethoxy]methyl}piperidin-3-yl)-3-methyl-2-oxo-1,3-benzodiazol-4-yl]hept-6-yn-1-yl methanesulfonate

To a solution of 3-[4-(7-hydroxyhept-1-yn-1-yl)-3-methyl-2-oxo-1,3-benzodiazol-1-yl]-1-{[2-(trimethylsilyl)ethoxy]methyl}piperidine-2,6-dione (Intermediate 75, 450 mg, 0.720 mmol) in DCM (6 mL) was added triethylamine (365 mg, 3.60 mmol) and MsCl (165 mg, 1.44 mmol) and the reaction mixture was stirred at room temperature for 1 h. MeOH (10 mL) was added and the volatiles were removed under reduced pressure. The residue was then chromatographed on a silica gel column, eluting with EtOAc/petroleum ether (60/40), to afford 7-[1-(2,6-dioxo-1-{[2-(trimethylsilyl)ethoxy]methyl}piperidin-3-yl)-3-methyl-2-oxo-1,3-benzodiazol-4-yl]hept-6-yn-1-yl methanesulfonate (Intermediate 76, 390 mg, 84%) as a yellow oil. Mass spec: m/z: 578.0 [M+H]⁺.

### Step 6

### Intermediate 77: 1-{7-[1-(2,6-dioxo-1-{[2-(trimethylsilyl)ethoxy]methyl}piperidin-3-yl)-3-methyl-2-oxo-1,3-benzodiazol-4-yl]hept-6-yn-1-yl}-3-methylindazole-6-carboxamide

A 10 mL round-bottom flask was charged with 7-[1-(2,6-dioxo-1-{[2-(trimethylsilyl)ethoxy]methyl}piperidin-3-yl)-3-methyl-2-oxo-1,3-benzodiazol-4-yl]hept-6-yn-1-yl methanesulfonate (Intermediate 76, 370 mg, 0.576 mmol), 3-methyl-1H-indazole-6-carboxamide (Intermediate 144, 121 mg, 0.691 mmol), K₂CO₃ (159 mg, 1.152 mmol), KI (95.6 mg, 0.576 mmol) in DMF (4 mL) and the reaction mixture was stirred at 60° C. for 4 h, under N₂. The crude reaction mixture was purified on a C18 silica column, eluting with MeCN/H₂O (1-2 drops formic acid) (45/55), to afford 1-{7-[1-(2,6-dioxo-1-{[2-(trimethylsilyl)ethoxy]methyl}piperidin-3-yl)-3-methyl-2-oxo-1,3-benzodiazol-4-yl]hept-6-yn-1-yl}-3-methylindazole-6-carboxamide (Intermediate 77, 80 mg, 17% d) as a yellow solid. Mass spec: m/z 657.0 [M+H]⁺.

### Step 7

### Intermediate 78: tert-butyl 6-(1-{7-[1-(2,6-dioxo-1-{[2-(trimethylsilyl)ethoxy]methyl}piperidin-3-yl)-3-methyl-2-oxo-1,3-benzodiazol-4-yl]hept-6-yn-1-yl}-3-methylindazole-6-amido)-2-[(2R)-1-methylpyrrolidin-2-yl]pyrrolo[3,2-c]pyridine-1-carboxylate

A 5 mL round-bottom flask was charged with 1-{7-[1-(2,6-dioxo-1-{[2-(trimethylsilyl)ethoxy]methyl}piperidin-3-yl)-3-methyl-2-oxo-1,3-benzodiazol-4-yl]hept-6-yn-1-yl}-3-methylindazole-6-carboxamide (Intermediate 77, 80.0 mg, 0.097 mmol), tert-butyl 6-bromo-2-[(2R)-1-methylpyrrolidin-2-yl]pyrrolo[3,2-c]pyridine-1-carboxylate (Intermediate 1, 40.7 mg, 0.107 mmol), Pd₂(dba)₃ (8.92 mg, 0.010 mmol), Xantphos (11.3 mg, 0.019 mmol), Cs₂CO₃ (63.5 mg, 0.194 mmol) in 1,4-dioxane (3 mL), under N₂ and the mixture was stirred at 60° C. for 3 h. The volatiles were removed under reduced pressure and the residue was purified by preparative TLC, eluting with DCM/MeOH (95/5), to afford tert-butyl 6-(1-{7-[1-(2,6-dioxo-1-{[2-(trimethylsilyl)ethoxy]methyl}piperidin-3-yl)-3-methyl-2-oxo-1,3-benzodiazol-4-yl]hept-6-yn-1-yl}-3-methylindazole-6-amido)-2-[(2R)-1-methylpyrrolidin-2-yl]pyrrolo[3,2-c]pyridine-1-carboxylate (Intermediate 78, 38 mg, 34%) as a yellow solid. Mass spec: m/z 956.0 [M+H]⁺.

### Step 8

### Example 11: 1-{7-[1-(2,6-dioxopiperidin-3-yl)-3-methyl-2-oxo-1,3-benzodiazol-4-yl]hept-6-yn-1-yl}-3-methyl-N-{2-[(2R)-1-methylpyrrolidin-2-yl]-1H-pyrrolo[3,2-c]pyridin-6-yl}indazole-6-carboxamide

To a solution of tert-butyl 6-(1-{7-[1-(2,6-dioxo-1-{[2-(trimethylsilyl)ethoxy]methyl}piperidin-3-yl)-3-methyl-2-oxo-1,3-benzodiazol-4-yl]hept-6-yn-1-yl}-3-methylindazole-6-amido)-2-[(2R)-1-methylpyrrolidin-2-yl]pyrrolo[3,2-c]pyridine-1-carboxylate (Intermediate 78, 38 mg, 0.033 mmol) in DCM (1 mL) was added MsOH (41.7 mg, 0.429 mmol) and the reaction mixture was stirred at room temperature for 2h. Triethylamine (39.5 mg, 0.390 mmol) and N,N′-dimethylethylenediamine (5.89 mg, 0.066 mmol) was then added and the reaction stirred for a further 2 h at room temperature. The reaction mixture was then purified by preparative TLC, eluting with DCM/MeOH (90/10), to afford crude product. The crude product was further purified by preparative HPLC (Column: XBridge Prep Shield RP C18 Column, 19*250 mm, 5 μm; Mobile Phase A: Water (0.1% FA), Mobile Phase B: MeOH; Flow rate: 25 mL/min; Gradient: 25% B to 55% B in 10 min), to afford 1-{7-[1-(2,6-dioxopiperidin-3-yl)-3-methyl-2-oxo-1,3-benzodiazol-4-yl]hept-6-yn-1-yl}-3-methyl-N-{2-[(2R)-1-methylpyrrolidin-2-yl]-1H-pyrrolo[3,2-c]pyridin-6-yl}indazole-6-carboxamide (Example 11, 2.1 mg, 8.2%) as a white solid. ¹H NMR (400 MHz, MeOH-d4) δ ppm 8.63 (s, 1H), 8.28 (s, 1H), 8.19 (s, 1H), 7.68 (d, J=8.0 Hz, 1H), 7.57 (d, J=8.4 Hz, 1H), 7.04 (d, J=7.6 Hz, 1H), 6.92-6.94 (m, 2H), 6.75 (s, 1H), 5.36-5.40 (m, 1H), 4.47-4.87 (m, 2H), 4.09-4.12 (m, 1H), 3.50-3.56 (m, 1H), 3.35-3.36 (m, 3H), 3.31-3.32 (m, 3H), 2.91-2.94 (m, 2H), 2.79-2.82 (m, 2H), 2.62 (s, 3H). 2.48-2.52 (m, 1H), 2.43-2.46 (m, 2H), 2.29-2.35 (m, 1H), 2.16-2.19 (m, 3H), 2.00-2.02 (m, 2H), 1.61-1.66 (m, 2H), 1.42-1.48 (m, 2H). Mass spec: m/z 726.4 [M+H]⁺.

### Example 12 was Synthesis Following Scheme 16

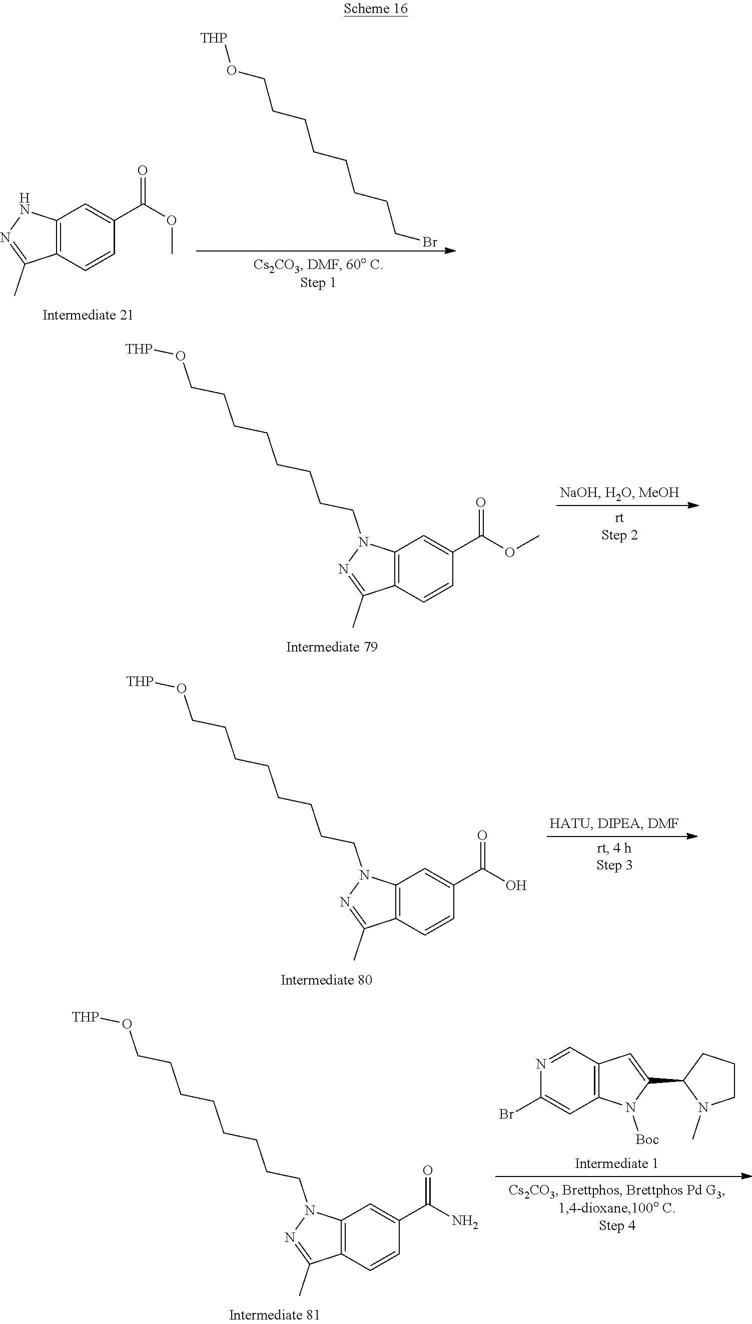

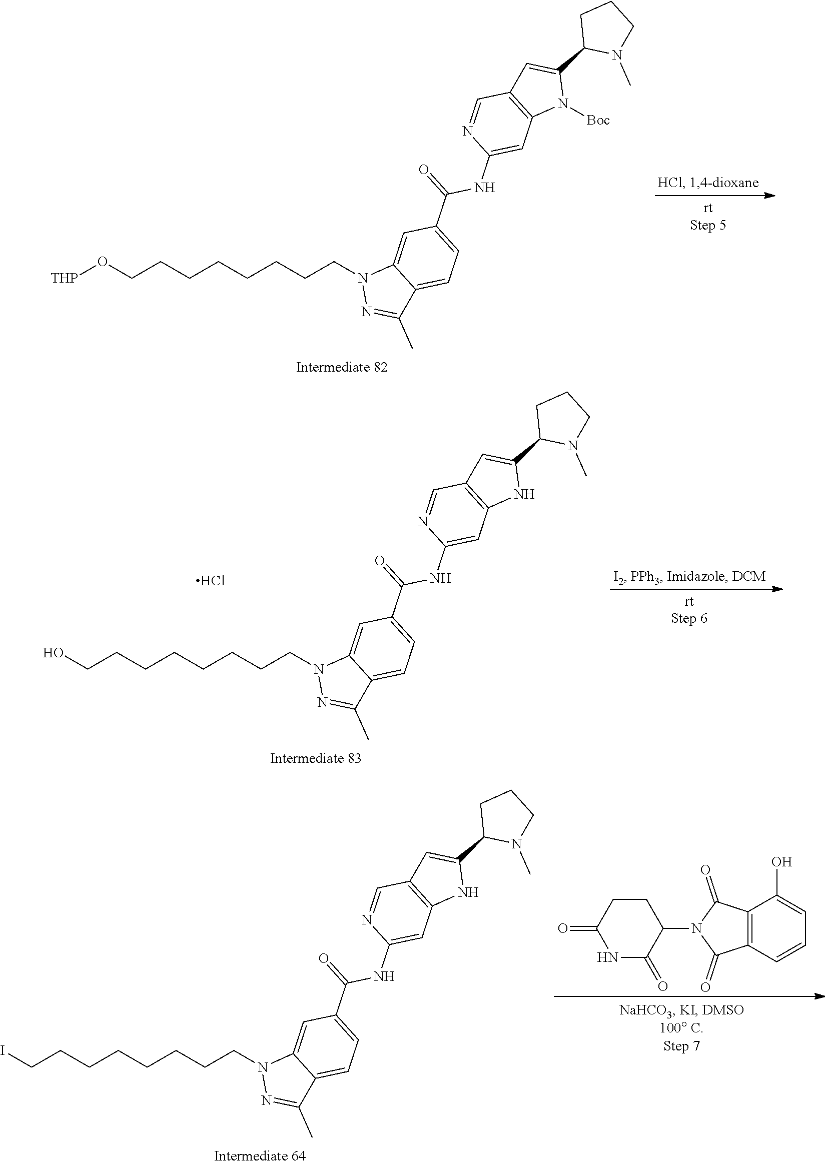

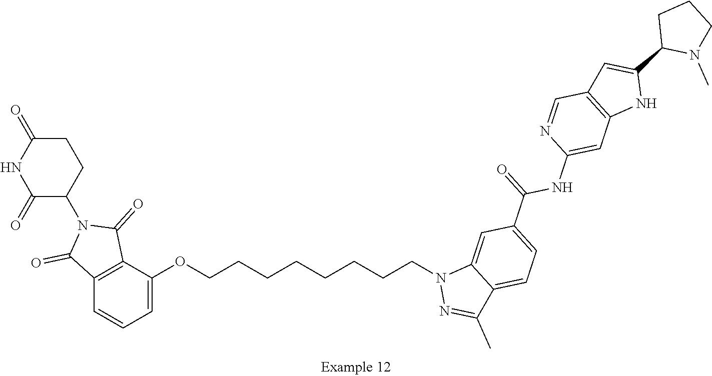 [SMILES: Cc1nn(CCCCCCCCOc2cccc3c2C(=O)N(C2CCC(=O)NC2=O)C3=O)c2cc(C(=O)Nc3cc4[nH]c([C@H]5CCCN5C)cc4cn3)ccc12]

### Step 1

### Intermediate 79: methyl 3-methyl-1-(8-((tetrahydro-2H-pyran-2-yl)oxy)octyl)-1H-indazole-6-carboxylate

To a solution of methyl 3-methyl-1H-indazole-6-carboxylate (Intermediate 21, 2.00 g, 9.46 mmol) in DMF (20 mL) was added 2-((8-bromooctyl)oxy)tetrahydro-2H-pyran (CAS No: 50816-20-1, 8.33 g, 28.4 mmol) and Cs₂CO₃ (6.17 g, 18.9 mmol) and the mixture was stirred at 60° C. for 2h. Water (20 mL) was added and the aqueous phase was extracted with EtOAc (3×50 mL). The organic layers were combined, washed with brine (150 mL), dried over anhydrous sodium sulfate, filtered and concentrated under reduced pressure. The residue was chromatographed on a silica gel column, eluting with EtOAc/petroleum ether (1/9), to afford methyl 3-methyl-1-(8-((tetrahydro-2H-pyran-2-yl)oxy)octyl)-1H-indazole-6-carboxylate (Intermediate 79, 3.50 g 83%) as a yellow oil. Mass spec: m/z 403.0 [M+H]⁺.

### Step 2

### Intermediate 80: 3-methyl-1-(8-((tetrahydro-2H-pyran-2-yl)oxy)octyl)-1H-indazole-6-carboxylic acid

To a solution of methyl 3-methyl-1-(8-((tetrahydro-2H-pyran-2-yl)oxy)octyl)-1H-indazole-6-carboxylate (Intermediate 79, 3.50 g, 7.83 mmol) in MeOH (40 mL) and H₂O (27 mL), was added NaOH (12.5 g, 313 mmol) and the mixture was stirred at room temperature for 2 h. The pH value of the mixture was adjusted to pH 6 by the dropwise addition of dilute aqueous HCl (1M). The mixture was then extracted with CHCl₃/iPrOH (3/1, 3×100 mL) and the organic layers were combined, washed with brine (300 mL), dried over anhydrous sodium sulfate, filtered and concentrated under reduced pressure to afford 3-methyl-1-(8-((tetrahydro-2H-pyran-2-yl)oxy)octyl)-1H-indazole-6-carboxylic acid (Intermediate 80, 3.00 g 84%) as a yellow oil, which was used in the next step without further purification. Mass spec: m/z 389.0 [M+H]⁺.

### Step 3

### Intermediate 81: 3-methyl-1-(8-((tetrahydro-2H-pyran-2-yl)oxy)octyl)-1H-indazole-6-carboxamide

To a solution of 3-methyl-1-(8-((tetrahydro-2H-pyran-2-yl)oxy)octyl)-1H-indazole-6-carboxylic acid (Intermediate 80, 3.00 g, 6.56 mmol) in DMF (40 mL) was added HATU (3.74 g, 9.85 mmol), DIPEA (5.09 g, 39.4 mmol) and NH₄Cl (1.40 g, 26.3 mmol) and the reaction mixture was stirred at room temperature for 4h. Water (50 mL) was added and the mixture was extracted with EtOAc (3×100 mL) and the organic layers were combined, washed with brine (300 mL), dried over anhydrous sodium sulfate, filtered and concentrated under reduced pressure. The residue was chromatographed on a silica gel column, eluting with MeOH/DCM (5/95) to afford 3-methyl-1-(8-((tetrahydro-2H-pyran-2-yl)oxy)octyl)-1H-indazole-6-carboxamide (Intermediate 81, 3.00 g 94%) as a yellow oil. Mass spec: m/z 388.0 [M+H]⁺.

### Step 4

### Intermediate 82: tert-butyl 6-(3-methyl-1-(8-((tetrahydro-2H-pyran-2-yl)oxy)octyl)-1H-indazole-6-carboxamido)-2-((R)-1-methylpyrrolidin-2-yl)-1H-pyrrolo[3,2-c]pyridine-1-carboxylate

To a solution of 3-methyl-1-(8-((tetrahydro-2H-pyran-2-yl)oxy)octyl)-1H-indazole-6-carboxamide (Intermediate 81, 1.00 g, 2.32 mmol) in 1,4-dioxane (50 mL) was added tert-butyl (R)-6-bromo-2-(1-methylpyrrolidin-2-yl)-1H-pyrrolo[3,2-c]pyridine-1-carboxylate (Intermediate 1, 1.28 g, 3.02 mmol), Brettphos (748 mg, 1.39 mmol), Brettphos Pd G₃ (632 mg, 0.697 mmol) and Cs₂CO₃ (1.51 g, 4.64 mmol) an the reaction was stirred at 100° C. for 2 days. The resulting crude was then chromatographed on a silica gel column, eluting with MeOH/DCM (5/95), to afford tert-butyl 6-(3-methyl-1-(8-((tetrahydro-2H-pyran-2-yl)oxy)octyl)-1H-indazole-6-carboxamido)-2-((R)-1-methylpyrrolidin-2-yl)-1H-pyrrolo[3,2-c]pyridine-1-carboxylate (Intermediate 82, 900 mg 50.77%) as a yellow oil. Mass spec: m/z 687.0 [M+H]⁺.

### Step 5

### Intermediate 83: (R)-1-(8-hydroxyoctyl)-3-methyl-N-(2-(1-methylpyrrolidin-2-yl)-1H-pyrrolo[3,2-c]pyridin-6-yl)-1H-indazole-6-carboxamide hydrochloride

To a solution of tert-butyl 6-(3-methyl-1-(8-((tetrahydro-2H-pyran-2-yl)oxy)octyl)-1H-indazole-6-carboxamido)-2-((R)-1-methylpyrrolidin-2-yl)-1H-pyrrolo[3,2-c]pyridine-1-carboxylate (Intermediate 82, 850 mg, 1.11 mmol) in 1,4-dioxane (15 mL) was added concentrated HCl (2 mL) and the mixture was stirred at room temperature for 2 h. The volatiles were then removed under reduced pressure to afford (R)-1-(8-hydroxyoctyl)-3-methyl-N-(2-(1-methylpyrrolidin-2-yl)-1H-pyrrolo[3,2-c]pyridin-6-yl)-1H-indazole-6-carboxamide hydrochloride (Intermediate 83, 550 mg) as a yellow solid, which was used in the next step without further purification. Mass spec: m/z: 503.0 [M+H]⁺.

### Step 6

### Intermediate 64: (R)-1-(8-iodooctyl)-3-methyl-N-(2-(1-methylpyrrolidin-2-yl)-1H-pyrrolo[3,2-c]pyridin-6-yl)-1H-indazole-6-carboxamide

To a solution of (R)-1-(8-hydroxyoctyl)-3-methyl-N-(2-(1-methylpyrrolidin-2-yl)-1H-pyrrolo[3,2-c]pyridin-6-yl)-1H-indazole-6-carboxamide hydrochloride (Intermediate 83, 450 mg, 0.806 mmol) in DCM (10 mL) was added PPh₃ (634 mg, 2.42 mmol) and imidazole (494 mg, 7.25 mmol), under N₂·I₂ (675 mg, 2.66 mmol) was then added and the mixture was stirred at room temperature for 3h. Water (10 mL) was added and the mixture was extracted with EtOAc (3×20 mL). The organic layers were combined, washed with brine (50 mL), dried over anhydrous sodium sulfate, filtered and concentrated under reduced pressure. The residue was chromatographed on a silica gel column, eluting with EtOAc/petroleum ether (3/7), to afford (R)-1-(8-iodooctyl)-3-methyl-N-(2-(1-methylpyrrolidin-2-yl)-1H-pyrrolo[3,2-c]pyridin-6-yl)-1H-indazole-6-carboxamide (Intermediate 64, 280 mg 51.06%) as a yellow solid. Mass spec: m/z 613.0 [M+H]⁺.

### Step 7

### Example 12: 1-(8-((2-(2,6-dioxopiperidin-3-yl)-1,3-dioxoisoindolin-4-yl)oxy)octyl)-3-methyl-N-(2-((R)-1-methylpyrrolidin-2-yl)-1H-pyrrolo[3,2-c]pyridin-6-yl)-1H-indazole-6-carboxamide

To a solution of(R)-1-(8-iodooctyl)-3-methyl-N-(2-(1-methylpyrrolidin-2-yl)-1H-pyrrolo[3,2-c]pyridin-6-yl)-1H-indazole-6-carboxamide (Intermediate 64, 80.0 mg, 0.118 mmol) in DMSO (2 mL) was added 2-(2,6-dioxopiperidin-3-yl)-4-hydroxyisoindoline-1,3-dione (CAS No: 5054-59-1, 32.2 mg, 0.118 mmol), NaHCO₃ (29.6 mg, 0.354 mmol) and KI (19.5 mg, 0.118 mmol) and the reaction was stirred overnight at 100° C. The resulting crude was chromatographed on a C18 silica column, eluting with water/MeCN (5/5) to afford crude product (40 mg). This was further purified by preparative HPLC (Column: YMC-Actus Triart C18 ExRs, 30*150 mm, 5 μm; Mobile Phase A: Water (10 mmol/L NH₄HCO₃), Mobile Phase B: MeCN; Flow rate: 60 mL/min; Gradient: 38% B to 68% B in 7 min, 68% B to 81% B in 10 min) to afford 1-(8-((2-(2,6-dioxopiperidin-3-yl)-1,3-dioxoisoindolin-4-yl)oxy)octyl)-3-methyl-N-(2-((R)-1-methylpyrrolidin-2-yl)-1H-pyrrolo[3,2-c]pyridin-6-yl)-1H-indazole-6-carboxamide (Example 12, 7.00 mg 7.8%) as a white solid. ¹H NMR (400 MHz, DMSO-d6) δ ppm as 11.38 (s, 1H), 11.10 (s, 1H), 10.54 (s, 1H), 8.52 (s, 1H), 8.44 (s, 1H), 8.25 (s, 1H), 7.72-7.80 (m, 3H), 7.41-7.48 (m, 2H), 6.41-6.53 (m, 1H), 5.05-5.09 (m, 1H), 4.41 (t, J=6.8 Hz, 2H), 4.16 (t, J=6.4 Hz, 2H), 3.16-3.17 (m, 1H), 2.85-2.95 (m, 1H), 2.55-2.65 (m, 2H), 2.45-2.55 (m, 2H), 2.20-2.25 (m, 1H), 2.15-2.20 (m, 4H), 1.65-2.01 (m, 10H) 1.40-1.55 (m, 2H), 1.25-1.30 (m, 6H). Mass spec: m/z 759.5 [M+H]⁺.

### Example 13 was Synthesised Following Scheme 17

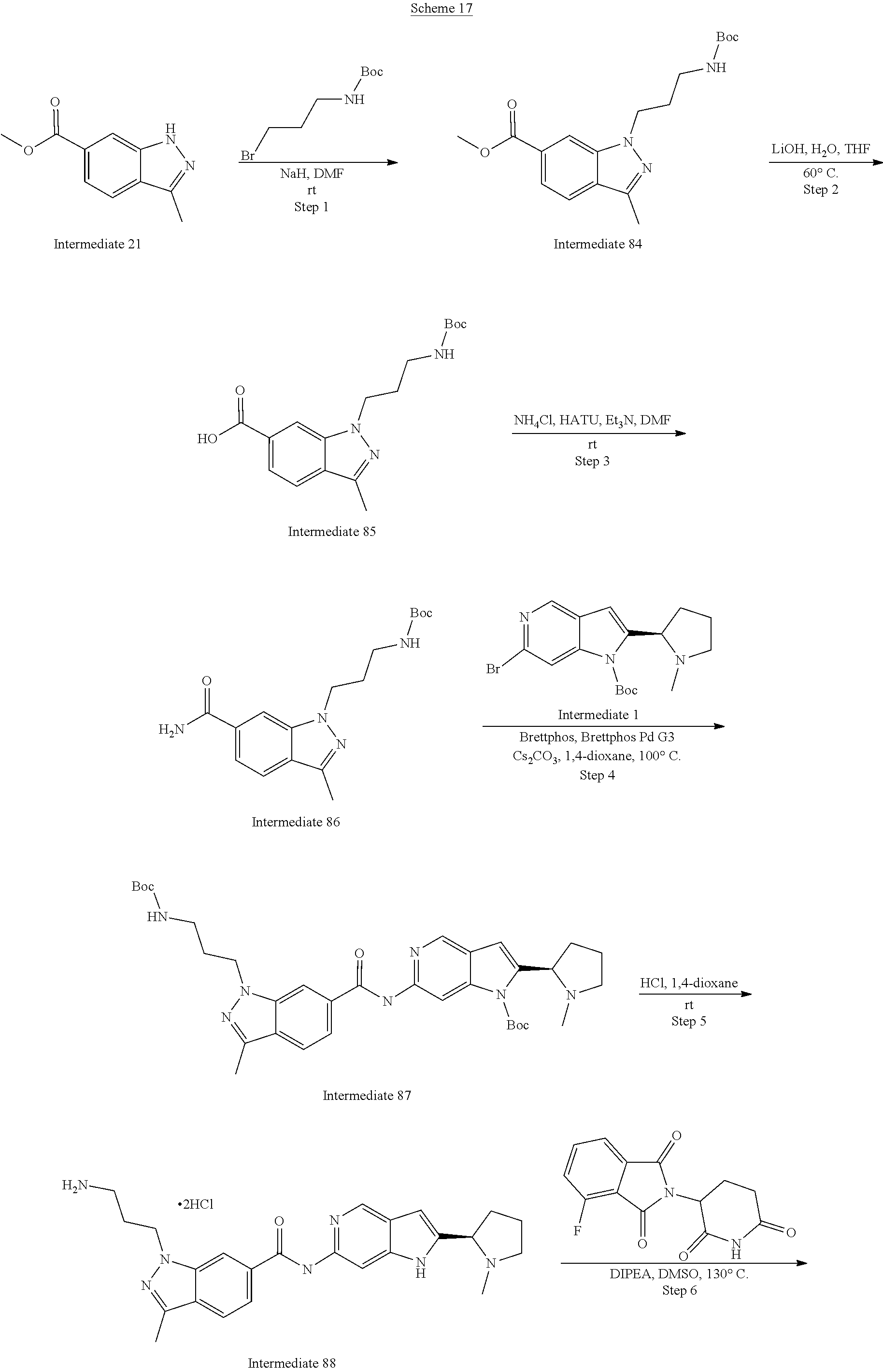

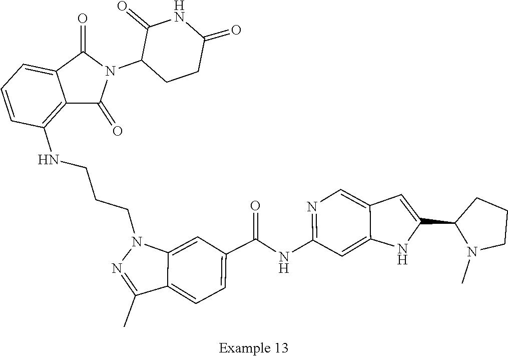 [SMILES: Cc1nn(CCCNc2cccc3c2C(=O)N(C2CCC(=O)NC2=O)C3=O)c2cc(C(=O)Nc3cc4[nH]c([C@H]5CCCN5C)cc4cn3)ccc12]

### Step 1

### Intermediate 84: methyl 1-(3-((tert-butoxycarbonyl)amino)propyl)-3-methyl-1H-indazole-6-carboxylate

To a solution of methyl 3-methyl-1H-indazole-6-carboxylate (Intermediate 21, 500 mg, 2.50 mmol) in DMF (8 mL) was added NaH (60% in mineral oil, 150 mg, 3.75 mmol) at 0° C. The reaction was then stirred for 30 min at 0° C. tert-Butyl (3-bromopropyl)carbamate (CAS No: 83948-53-2, 751 mg, 3.00 mmol) was then added and the reaction was stirred at room temperature for 2h. Water (20 mL) was added and the aqueous phase was extracted with EtOAc (2×50 mL). The organic layers were combined, washed with brine, dried over anhydrous sodium sulfate, filtered and concentrated under reduced pressure. The residue was then chromatographed on a silica gel column, eluting with MeOH/DCM (2/98), to afford methyl 1-(3-((tert-butoxycarbonyl)amino)propyl)-3-methyl-1H-indazole-6-carboxylate (Intermediate 84, 270 mg, 28%) as a colorless oil. Mass spec: m/z: 348.2 [M+H]⁺.

### Step 2

### Intermediate 85: 1-(3-((tert-butoxycarbonyl)amino)propyl)-3-methyl-1H-indazole-6-carboxylic acid

To a stirred solution of methyl 1-(3-((tert-butoxycarbonyl)amino)propyl)-3-methyl-1H-indazole-6-carboxylate (Intermediate 84, 454 mg, 1.18 mmol) in THF (8 mL) and water (2 mL) was added LiOH (141 mg, 5.88 mmol) and the resulting mixture was stirred at 60° C. for 2h. The pH value of the solution was adjusted to pH ~5 by dropwise addition of dilute aqueous HCl (1M) and the resulting mixture was extracted with EtOAc (2×50 mL). The combined organic layers were washed with brine and dried over anhydrous Na₂SO₄ and then concentrated under reduced pressure to afford 1-(3-((tert-butoxycarbonyl)amino)propyl)-3-methyl-1H-indazole-6-carboxylic acid (Intermediate 85, 230 mg, 52%) as a white solid. Mass spec: m/z: 334.1 [M+H]⁺.

### Step 3

### Intermediate 86: tert-butyl (3-(6-carbamoyl-3-methyl-1H-indazol-1-yl)propyl)carbamate

A 100 mL round-bottom flask was charged with 1-(3-((tert-butoxycarbonyl)amino)propyl)-3-methyl-1H-indazole-6-carboxylic acid (Intermediate 85, 332 mg, 0.896 mmol), NH₄Cl (240 mg, 4.48 mmol), DMF (6 mL), HATU (511 mg, 1.34 mmol), DIPEA (348 mg, 2.69 mmol) and the reaction was stirred overnight at room temperature. The mixture was chromatographed on a C18 silica column, eluting with MeCN/water (with 0.5% NH₄HCO₃) (3/7) to afford tert-butyl (3-(6-carbamoyl-3-methyl-1H-indazol-1-yl)propyl)carbamate (Intermediate 86, 280 mg, 89%) as a white solid. Mass spec: m/z: 333.2[M+H]⁺.

### Step 4

### Intermediate 87: tert-butyl (R)-6-(1-(3-((tert-butoxycarbonyl)amino)propyl)-3-methyl-1H-indazole-6-carboxamido)-2-(1-methylpyrrolidin-2-yl)-1H-pyrrolo[3,2-c]pyridine-1-carboxylate

A 100 mL round-bottom flask was charged with tert-butyl (3-(6-carbamoyl-3-methyl-1H-indazol-1-yl)propyl)carbamate (Intermediate 86, 120 mg, 0.343 mmol), tert-butyl (R)-6-bromo-2-(1-methylpyrrolidin-2-yl)-1H-pyrrolo[3,2-c]pyridine-1-carboxylate (Intermediate 1, 159 mg, 0.377 mmol), Brettphos (55.2 mg, 0.103 mmol), Brettphos Pd G3 (187 mg, 0.206 mmol), Cs₂CO₃ (559 mg, 1.72 mmol) in 1,4-dioxane (8 mL) and the reaction was stirred at 100° C. overnight, under N₂. Water (10 mL) was added and the aqueous phase was extracted with EtOAc (2×50 mL). The combined organic layers were washed with brine, dried over anhydrous Na₂SO₄, and concentrated under reduced pressure. The resulting residue was chromatographed on a silica gel column, eluting with MeOH/DCM (1/9), to afford tert-butyl (R)-6-(1-(3-((tert-butoxycarbonyl)amino)propyl)-3-methyl-1H-indazole-6-carboxamido)-2-(1-methylpyrrolidin-2-yl)-1H-pyrrolo[3,2-c]pyridine-1-carboxylate (Intermediate 87, 70 mg, 29.1%) as a light yellow oil. Mass spec: m/z: 632.4 [M+H]⁺.

### Step 5

### Intermediate 88: (R)-1-(3-aminopropyl)-3-methyl-N-(2-(1-methylpyrrolidin-2-yl)-1H-pyrrolo[3,2-c]pyridin-6-yl)-1H-indazole-6-carboxamide dihydrochloride

To a solution of tert-butyl (R)-6-(1-(3-((tert-butoxycarbonyl)amino)propyl)-3-methyl-1H-indazole-6-carboxamido)-2-(1-methylpyrrolidin-2-yl)-1H-pyrrolo[3,2-c]pyridine-1-carboxylate (Intermediate 87, 70 mg, 0.125 mmol) in 1,4-dioxane (5 mL) was added concentrated aqueous HCl (1 mL) and the reaction was stirred for 1 h at room temperature. The volatiles were then removed under reduced pressure to afford (R)-1-(3-aminopropyl)-3-methyl-N-(2-(1-methylpyrrolidin-2-yl)-1H-pyrrolo[3,2-c]pyridin-6-yl)-1H-indazole-6-carboxamide dihydrochloride (Intermediate 88, 70 mg, 97%) as an off-white solid, which was used in the next step without further purification. Mass spec: m/z: 433.2 [M+H]⁺.

### Step 6

### Example 13: 1-(3-((2-(2,6-dioxopiperidin-3-yl)-1,3-dioxoisoindolin-4-yl)amino)propyl)-3-methyl-N-(2-((R)-1-methylpyrrolidin-2-yl)-1H-pyrrolo[3,2-c]pyridin-6-yl)-1H-indazole-6-carboxamide

To a solution of (R)-1-(3-aminopropyl)-3-methyl-N-(2-(1-methylpyrrolidin-2-yl)-1H-pyrrolo[3,2-c]pyridin-6-yl)-1H-indazole-6-carboxamide dihydrochloride (Intermediate 88, 70 mg, 0.122 mmol), 2-(2,6-dioxopiperidin-3-yl)-4-fluoroisoindoline-1,3-dione (CAS No: 835616-60-9, 67.2 mg, 0.244 mmol) in DMSO (4 mL) was added DIPEA (94.3 mg, 0.732 mmol) and the reaction was stirred at 130° C. for 1 h. The reaction mixture was then purified by a C18 silica column, eluting with MeCN/water (3/2), to afford a crude product. This crude product was further purified by preparative HPLC (Column: XBridge Prep OBD C18 Column, 30*150 mm, 5 μm; Mobile Phase A: Water (with 10 mmol/L NH₄HCO₃), Mobile Phase B: MeCN; Flow rate: 60 mL/min; Gradient: 29% B to 59% B in 10 min) to afford 1-(3-((2-(2,6-dioxopiperidin-3-yl)-1,3-dioxoisoindolin-4-yl)amino)propyl)-3-methyl-N-(2-((R)-1-methylpyrrolidin-2-yl)-1H-pyrrolo[3,2-c]pyridin-6-yl)-1H-indazole-6-carboxamide (Example 13, 5.2 mg, 6.1%) as a yellow solid. ¹H NMR (400 MHz, MeOH-d4) δ ppm 8.54 (s, 1H), 8.20 (s, 1H), 8.14 (s, 1H), 7.82 (d, J=8.4 Hz, 1H), 7.67 (d, J=8.4 Hz, 1H), 7.41 (t, J=7.2 Hz, 1H), 6.92 (d, J=7.2 Hz, 1H), 6.82 (d, J=8.8 Hz, 1H), 6.55 (s, 1H), 4.60-4.63 (m, 2H), 3.37-3.50 (m, 1H), 3.28-3.33 (m, 4H), 2.59-2.64 (m, 6H), 2.33-2.46 (m, 7H), 2.05-2.08 (m, 2H), 1.97-2.01 (m, 2H). Mass spec: m/z: 688.6 [M+H]⁺.

### Example 14: 1-(8-{[2-(2,6-dioxopiperidin-3-yl)-1,3-dioxoisoindol-5-yl]amino}octyl)-3-methyl-N-{2-[(2R)-1-methylpyrrolidin-2-yl]-1H-pyrrolo[3,2-c]pyridin-6-yl}indazole-6-carboxamide

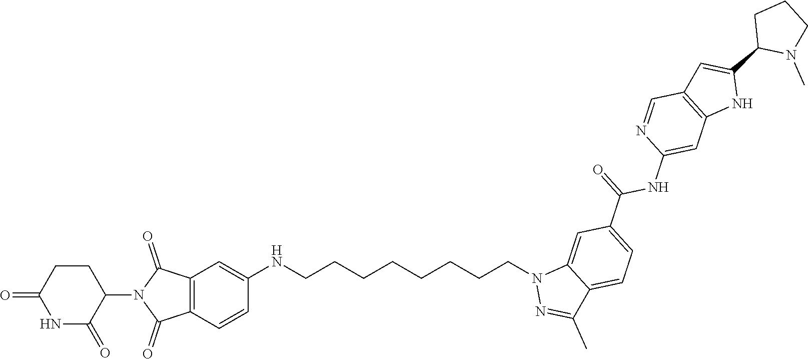 [SMILES: Cc1nn(CCCCCCCCNc2ccc3c(c2)C(=O)N(C2CCC(=O)NC2=O)C3=O)c2cc(C(=O)Nc3cc4[nH]c([C@H]5CCCN5C)cc4cn3)ccc12]

To a solution of 1-(8-aminooctyl)-3-methyl-N-{2-[(2R)-1-methylpyrrolidin-2-yl]-1H-pyrrolo[3,2-c]pyridin-6-yl}indazole-6-carboxamide dihydrochloride (Intermediate 35, 100 mg, 0.179 mmol) and 2-(2,6-dioxopiperidin-3-yl)-5-fluoroisoindole-1,3-dione (CAS No: 835616-61-0, 71.6 mg, 0.259 mmol) in DMSO (3 mL) was added DIPEA (258 mg, 1.99 mmol) and the reaction was stirred at 120° C. for 4h. the reaction mixture was then purified by preparative HPLC (Column: WelFlash C18, Regular C18 20-40m, 120 g; Mobile Phase A: Water (with 10 mmol/L NH₄HCO₃+0.05% NH₃·H₂O), Mobile Phase B: MeCN; Flow rate: 60 mL/min mL/min; Gradient: 41% B to 71% B in 30 min) to afford 1-(8-{[2-(2,6-dioxopiperidin-3-yl)-1,3-dioxoisoindol-5-yl]amino}octyl)-3-methyl-N-{2-[(2R)-1-methylpyrrolidin-2-yl]-1H-pyrrolo[3,2-c]pyridin-6-yl}indazole-6-carboxamide (Example 14, 56.6 mg, 41%) as a yellow solid. (400 MHz, DMSO-d6) δ ppm 11.40 (s, 1H), 11.05 (br, 1H), 10.55 (s, 1H), 8.53 (s, 1H), 8.44 (s, 1H), 8.26 (s, 1H), 7.73-7.80 (m, 2H), 7.54 (d, J=8.4 Hz, 1H), 7.05-7.08 (m, 1H), 6.92 (s, 1H), 6.80-6.82 (m, 1H), 6.41 (s, 1H), 5.00-5.05 (m, 1H), 4.39-4.42 (m, 2H), 3.08-3.17 (m, 3H), 2.80-2.95 (m, 1H), 2.50-2.60 (m, 5H), 2.26-2.28 (m, 1H), 2.08-2.18 (m, 4H), 1.83-2.01 (m, 6H), 1.52-1.55 (m, 2H), 1.20-1.40 (m, 9H). Mass spec: m/z: 758.5 [M+H]⁺.

### Example 15: 1-(8-{[2-(2,6-dioxopiperidin-3-yl)-1,3-dioxoisoindol-5-yl]amino}octyl)-3-methyl-N-{2-[(2S)-1-methylpyrrolidin-2-yl]-1H-pyrrolo[3,2-c]pyridin-6-yl}indazole-6-carboxamide

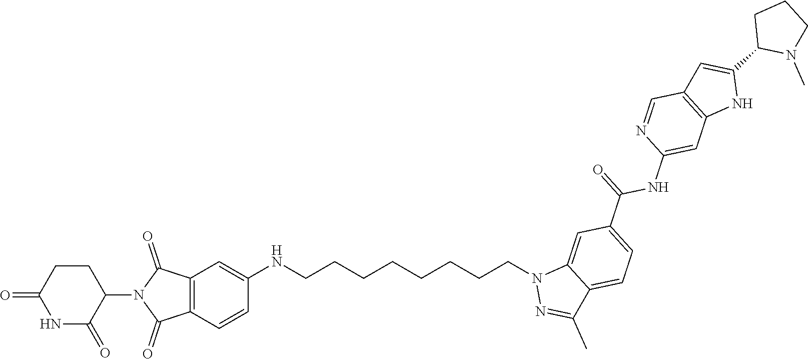 [SMILES: Cc1nn(CCCCCCCCNc2ccc3c(c2)C(=O)N(C2CCC(=O)NC2=O)C3=O)c2cc(C(=O)Nc3cc4[nH]c([C@@H]5CCCN5C)cc4cn3)ccc12]

Example 15 was made in an analogous manner to Example 14 using 1-(8-aminooctyl)-3-methyl-N-{2-[(2S)-1-methylpyrrolidin-2-yl]-1H-pyrrolo[3,2-c]pyridin-6-yl}indazole-6-carboxamide dihydrochloride (Intermediate 35) and 2-(2,6-dioxopiperidin-3-yl)-5-fluoroisoindole-1,3-dione (CAS No: 835616-61-0), to afford 1-(8-{[2-(2,6-dioxopiperidin-3-yl)-1,3-dioxoisoindol-5-yl]amino}octyl)-3-methyl-N-{2-[(2S)-1-methylpyrrolidin-2-yl]-1H-pyrrolo[3,2-c]pyridin-6-yl}indazole-6-carboxamide (Example 15). ¹H NMR (400 MHz, MeOH-d4) δ ppm 8.57 (s, 1H), 8.21-8.25 (m, 2H), 7.86 (d, J=8.4 Hz, 1H), 7.75 (d, J=8.4 Hz, 1H), 7.51 (d, J=8.8 Hz, 1H), 6.93 (s, 1H), 6.75-6.78 (m, 1H), 6.59 (s, 1H), 5.02-5.07 (m, 1H), 4.44-4.48 (m, 2H), 3.62-3.66 (m, 1H), 3.12-3.15 (m, 2H), 2.82-2.86 (m, 1H), 2.67-2.73 (m, 2H), 2.61 (s, 3H), 2.55-2.57 (m, 1H), 2.36-2.43 (m, 4H), 2.11-2.16 (m, 3H), 1.94-2.01 (m, 3H), 1.61-1.68 (m, 2H), 1.42-1.53 (m, 9H). Mass spec: m/z: 758.65 [M+H]⁺.

### Example 16: 1-(8-{[2-(2,6-dioxopiperidin-3-yl)-1-oxo-3H-isoindol-4-yl]amino}octyl)-3-methyl-N-{2-[(2R)-1-methylpyrrolidin-2-yl]-1H-pyrrolo[3,2-c]pyridin-6-yl}indazole-6-carboxamide

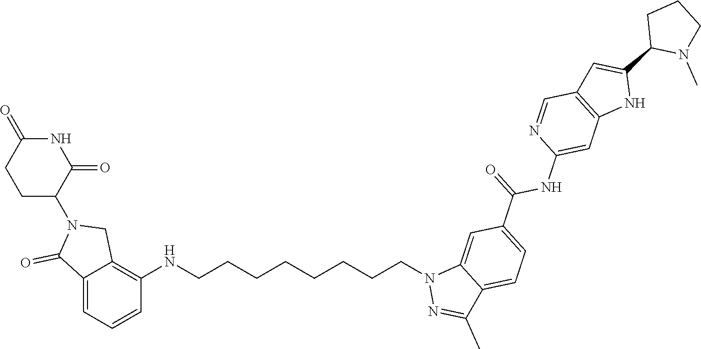 [SMILES: Cc1nn(CCCCCCCCNc2cccc3c2CN(C2CCC(=O)NC2=O)C3=O)c2cc(C(=O)Nc3cc4[nH]c([C@H]5CCCN5C)cc4cn3)ccc12]

To a solution of 1-(8-aminooctyl)-3-methyl-N-{2-[(2R)-1-methylpyrrolidin-2-yl]-1H-pyrrolo[3,2-c]pyridin-6-yl}indazole-6-carboxamide (Intermediate 35, 80.0 mg, 0.143 mmol) and 3-(4-bromo-1-oxo-3H-isoindol-2-yl)piperidine-2,6-dione (CAS No: 2093387-36-9, 67 mg, 0.21 mmol) in 1,4-dioxane (2 mL) was added Cs₂CO₃ (156 mg, 0.477 mmol), Pd-PEPPSI-IHept-Cl (CAS No: 1814936-54-3, 15.5 mg, 0.016 mmol) and the reaction was stirred overnight at 100° C., under N₂. The reaction mixture was then purified using preparative HPLC (Column: XselectCSH Prep OBD C18 Column, 30*150 mm, 5 μm; Mobile Phase A: Water (with 0.1% formic acid), Mobile Phase B: MeCN; Flow rate: 60 mL/min mL/min; Gradient: 14% B to 34% B in 8 min) to afford get 1-(8-{[2-(2,6-dioxopiperidin-3-yl)-1-oxo-3H-isoindol-4-yl]amino}octyl)-3-methyl-N-{2-[(2R)-1-methylpyrrolidin-2-yl]-1H-pyrrolo[3,2-c]pyridin-6-yl}indazole-6-carboxamide (Example 16, 3.6 mg, 3.2%) as a white solid. ¹H-NMR (400 MHz, MeOH-d4) δ ppm 8.60 (s, 1H), 8.21-8.25 (m, 2H), 7.83 (d, J=8.4 Hz, 1H), 7.74 (d, J=8.4 Hz, 1H), 7.28 (t, J=8.0 Hz, 1H), 7.05 (d, J=7.2 Hz, 1H), 6.77 (d, J=8.0 Hz, 1H), 6.59 (s, 1H), 5.14-5.18 (m, 1H), 4.43 (t, J=6.8 Hz, 2H), 4.26 (s, 2H), 3.60-3.70 (m, 1H), 3.15 (t, J=7.0 Hz, 2H), 2.85-2.95 (m, 1H), 2.70-2.80 (m, 1H), 2.55-2.60 (m, 4H), 2.35-2.44 (m, 5H), 1.91-2.18 (m, 7H), 1.55-1.65 (m, 2H), 1.20-1.40 (m, 9H). Mass spec: m/z: 744.5 [M+H]⁺.

### Example 17: 1-(8-{[2-(2,6-dioxopiperidin-3-yl)-1-oxo-3H-isoindol-4-yl]amino}octyl)-3-methyl-N-{2-[(2S)-1-methylpyrrolidin-2-yl]-1H-pyrrolo[3,2-c]pyridin-6-yl}indazole-6-carboxamide

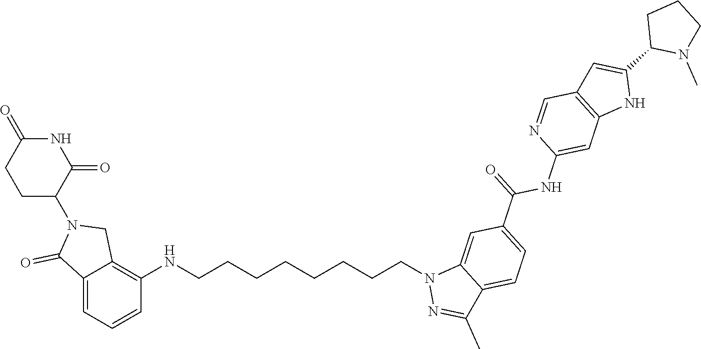 [SMILES: Cc1nn(CCCCCCCCNc2cccc3c2CN(C2CCC(=O)NC2=O)C3=O)c2cc(C(=O)Nc3cc4[nH]c([C@@H]5CCCN5C)cc4cn3)ccc12]

Example 17 was synthesised in an analogous manner to Example 16 using 1-(8-aminooctyl)-3-methyl-N-{2-[(2S)-1-methylpyrrolidin-2-yl]-1H-pyrrolo[3,2-c]pyridin-6-yl}indazole-6-carboxamide (Intermediate 35) as the amine and 3-(4-bromo-1-oxo-3H-isoindol-2-yl)piperidine-2,6-dione (CAS no: 2093387-36-9) as the aryl bromide, to afford 1-(8-{[2-(2,6-dioxopiperidin-3-yl)-1-oxo-3H-isoindol-4-yl]amino}octyl)-3-methyl-N-{2-[(2S)-1-methylpyrrolidin-2-yl]-1H-pyrrolo[3,2-c]pyridin-6-yl}indazole-6-carboxamide (Example 17, 4.6 mg, 6.1%) as a white solid. ¹H NMR (400 MHz, CD₃OD) δ ppm 8.55 (s, 1H), 8.23 (s, 1H), 8.19 (s, 1H), 7.80 (d, J=8.4 Hz, 1H), 7.73 (d, J=7.6 Hz, 1H), 7.28 (t, J=8.0 Hz, 1H), 7.05 (d, J=7.2 Hz, 1H), 6.75 (d, J=8.4 Hz, 1H), 6.52 (s, 1H), 5.12-5.17 (m, 1H), 4.40 (t, J=7.2 Hz, 2H), 4.26 (s, 2H), 3.45-3.47 (m, 1H), 3.25-3.30 (m, 1H), 3.12-3.15 (m, 2H), 2.81-2.89 (m, 1H), 2.77-2.78 (m, 1H), 2.58 (s, 3H), 2.40-2.44 (m, 2H), 2.25-2.31 (m, 4H), 2.11-2.16 (m, 1H), 1.92-2.05 (m, 2H), 1.88-1.90 (m, 2H), 1.56-1.60 (m, 2H), 1.29-1.31 (m, 9H). Mass spec: m/z: 744.5 [M+H]⁺.

### Example 18: N-(3-chloro-2-((R)-1-methylpyrrolidin-2-yl)-1H-pyrrolo[3,2-c]pyridin-6-yl)-1-(8-((2-(2,6-dioxopiperidin-3-yl)-1,3-dioxoisoindolin-4-yl)amino)octyl)-3-methyl-1H-indazole-6-carboxamide

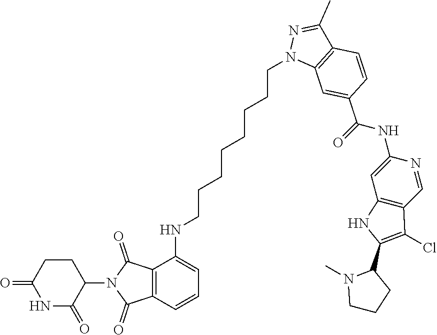

To a solution of 1-(8-((2-(2,6-dioxopiperidin-3-yl)-1,3-dioxoisoindolin-4-yl)amino)octyl)-3-methyl-N-(2-((R)-1-methylpyrrolidin-2-yl)-1H-pyrrolo[3,2-c]pyridin-6-yl)-1H-indazole-6-carboxamide (Example 3, 100 mg, 0.125 mmol) in DMF (2 mL) was added NCS (21.8 mg, 0.163 mmol) and the mixture was stirred at room temperature overnight under a nitrogen atmosphere. The resulting crude was purified by preparative HPLC (Column: XBridge Prep RP OBD C18 Column, 30*150 mm, 5 μm; Mobile Phase A: Water (10 mmol/L NH₄HCO₃), Mobile Phase B: MeCN; Flow rate: 60 mL/min; Gradient: 60% B to 90% B in 7 min) to afford N-(3-chloro-2-((R)-1-methylpyrrolidin-2-yl)-1H-pyrrolo[3,2-c]pyridin-6-yl)-1-(8-((2-(2,6-dioxopiperidin-3-yl)-1,3-dioxoisoindolin-4-yl)amino)octyl)-3-methyl-1H-indazole-6-carboxamide (Example 18, 23.5 mg, 23%) as a yellow solid. 1H NMR (400 MHz, DMSO-d6) δ ppm 11.72 (s, 1H), 11.09 (s, 1H), 10.70 (s, 1H), 8.53 (s, 1H), 8.45 (s, 1H), 8.32 (s, 1H), 7.73-7.80 (m, 2H), 7.54 (t, J=7.6 Hz, 1H), 6.99-7.04 (m, 2H), 6.49 (t, J=5.2 Hz, 1H), 5.02-5.07 (m, 1H), 4.40 (t, J=6.4 Hz, 2H), 3.50-3.60 (m, 1H), 3.20-3.32 (m, 3H), 2.80-2.93 (m, 1H), 2.60-2.65 (m, 1H), 2.50-2.55 (m, 3H), 2.25-2.35 (m, 1H), 2.15-2.22 (m, 4H), 1.80-2.05 (m, 6H), 1.40-1.45 (m, 2H), 1.30 (s, 9H). Mass spec: m/z 792.4 [M+H]⁺.

### Example 19 was Synthesized Following Scheme 18

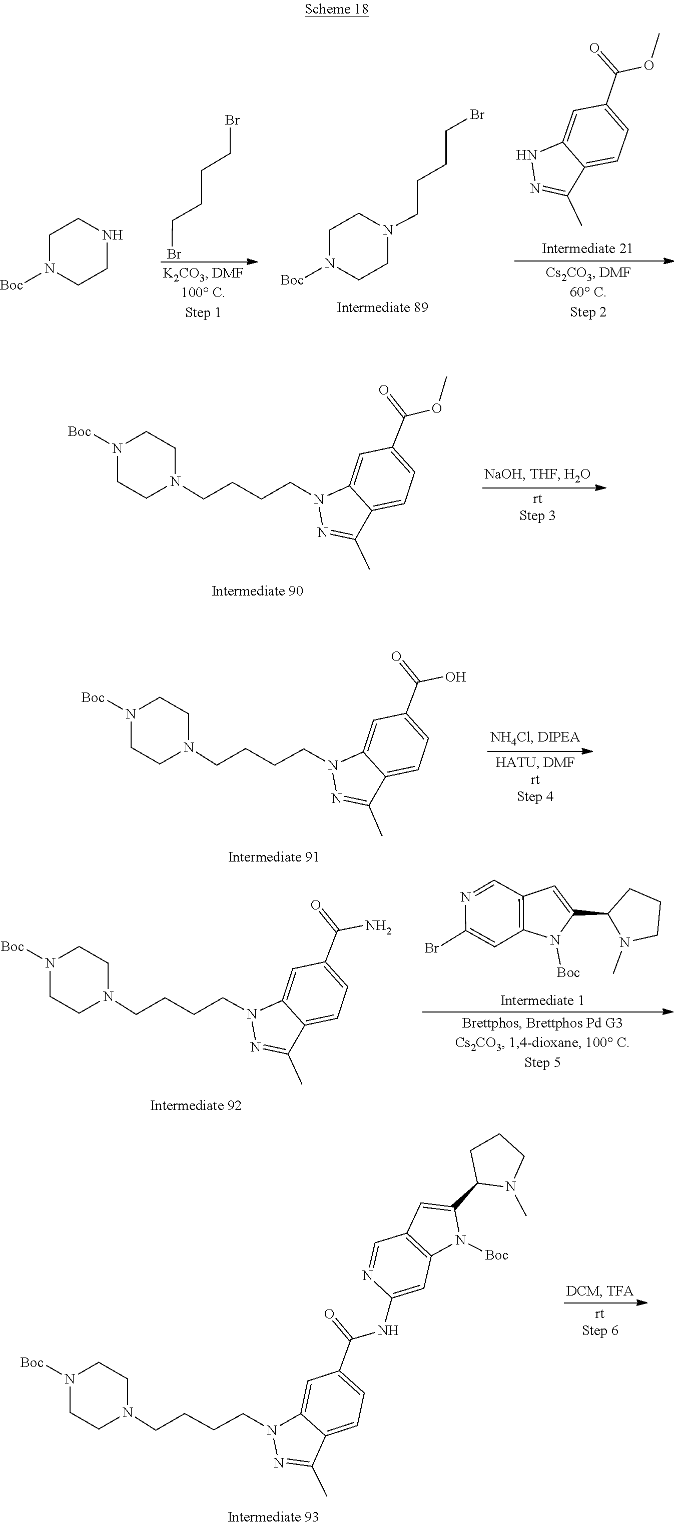

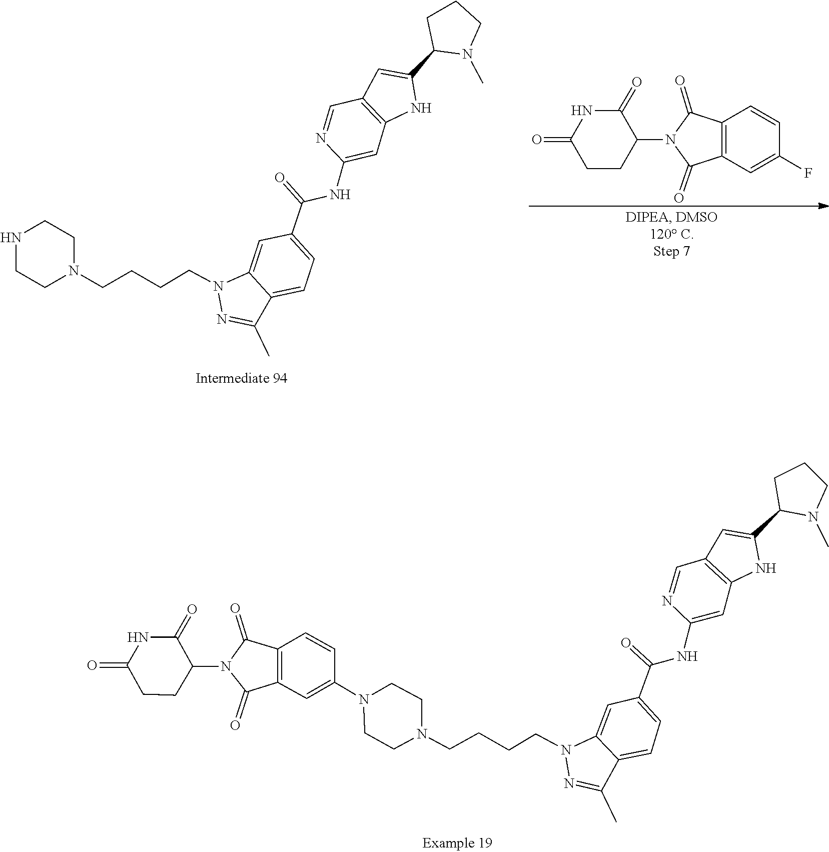 [SMILES: Cc1nn(CCCCN2CCNCC2)c2cc(C(=O)Nc3cc4[nH]c([C@H]5CCCN5C)cc4cn3)ccc12 ; O=C1CCC(N2C(=O)c3ccc(F)cc3C2=O)C(=O)N1 ; Cc1nn(CCCCN2CCN(c3ccc4c(c3)C(=O)N(C3CCC(=O)NC3=O)C4=O)CC2)c2cc(C(=O)Nc3cc4[nH]c([C@H]5CCCN5C)cc4cn3)ccc12]

### Step 1

### Intermediate 89: tert-butyl 4-(4-bromobutyl)piperazine-1-carboxylate

To a solution of tert-butyl piperazine-1-carboxylate (CAS No: 57260-71-6, 1.00 g, 5.10 mmol) in dimethyformamide (10 mL) was added 1,4-dibromobutane (CAS No: 110-52-1, 1.10 g, 5.10 mmol) and potassium carbonate (1.06 g, 7.65 mmol) then the reaction was stirred at 100° C. for 5 h. The mixture was quenched with water (100 mL) and extracted with ethyl acetate (3×100 mL). The combined organic layers were washed with brine, dried over anhydrous Na₂SO₄, filtered, and concentrated in vacuo to afford tert-butyl 4-(4-bromobutyl)piperazine-1-carboxylate (Intermediate 89, 1.50 g, 91%) as a white solid. Mass spec: m/z: 321 [M+H]⁺.

### Step 2

### Intermediate 90: methyl 1-{4-[4-(tert-butoxycarbonyl)piperazin-1-yl]butyl}-3-methylindazole-6-carboxylate

To a solution of tert-butyl 4-(4-bromobutyl)piperazine-1-carboxylate (Intermediate 89, 500 mg, 1.55 mmol) in dimethylformamide (15 mL) was added methyl 3-methyl-1H-indazole-6-carboxylate (Intermediate 21, 296 mg, 1.55 mmol) and cesium carbonate (1.52 g, 4.66 mmol) and the reaction was stirred at 60° C. for 16 h. The mixture was quenched with water (100 mL) and extracted with ethyl acetate (3×100 mL). The combined organic layers were washed with brine, dried over anhydrous Na₂SO₄, filtered and concentrated in vacuo. The crude material was purified by combi-flash column chromatography, by eluting with 2% MeOH in DCM, to afford methyl 1-{4-[4-(tert-butoxycarbonyl)piperazin-1-yl]butyl}-3-methylindazole-6-carboxylate (Intermediate 90, 200 mg, 30%) as a white solid. Mass spec: m/z: 431 [M+H]⁺.

### Step 3

### Intermediate 91: 1-{4-[4-(tert-butoxycarbonyl)piperazin-1-yl]butyl}-3-methylindazole-6-carboxylic acid

To a solution of methyl-1-{4-[4-(tert-butoxycarbonyl)piperazin-1-yl]butyl}-3-methylindazole-6-carboxylate (Intermediate 90, 200 mg, 0.460 mmol) in tetrahydrofuran (4 mL) was added water (4 mL) and sodium hydroxide (81.3 mg, 2.03 mmol) and the reaction was stirred at room temperature for 16 h. The pH of the reaction mixture was adjusted to pH 5 by the dropwise addition of concentrated aqueous HCl and then extracted with ethyl acetate (3×100 mL). The combined organic phases were then dried over anhydrous sodium sulfate and concentrated in vacuo to afford 1-{4-[4-(tert-butoxycarbonyl)piperazin-1-yl]butyl}-3-methylindazole-6-carboxylic acid (Intermediate 91, 180 mg, 89%) as a white solid, which was used in the next step without further purification. Mass spec: m/z: 417 [M+H]⁺.

### Step 4

### Intermediate 92: tert-butyl 4-[4-(6-carbamoyl-3-methylindazol-1-yl)butyl]piperazine-1-carboxylate

To a solution of 1-{4-[4-(tert-butoxycarbonyl)piperazin-1-yl]butyl}-3-methylindazole-6-carboxylic acid (Intermediate 91, 180 mg, 0.427 mmol) in dimethylformamide (5 mL) was added DIPEA (331 mg, 2.56 mmol), HATU (243 mg, 0.640 mmol) and NH₄Cl (91.4 mg, 1.70 mmol) and the reaction was stirred at room temperature for 16 h. The mixture was quenched with water (100 mL) and the aqueous phase extracted with ethyl acetate (3×100 mL). The combined organic layers were washed with brine, dried over anhydrous Na₂SO₄, filtered and concentrated in vacuo. The crude material was purified by combi-flash column chromatography, by eluting with 9% MeOH in DCM, to afford tert-butyl 4-[4-(6-carbamoyl-3-methylindazol-1-yl)butyl]piperazine-1-carboxylate (Intermediate 92, 180 mg, 98%) as a yellow solid. Mass spec: m/z: 416 [M+H]⁺.

### Step 5

### Intermediate 93: tert-butyl 4-[4-(6-{[1-(tert-butoxycarbonyl)-2-[(2R)-1-methylpyrrolidin-2-yl]pyrrolo[3,2-c]pyridin-6-yl]carbamoyl}-3-methylindazol-1-yl)butyl]piperazine-1-carboxylate

To a solution of tert-butyl 4-[4-(6-carbamoyl-3-methylindazol-1-yl)butyl]piperazine-1-carboxylate (Intermediate 92, 140 mg, 0.337 mmol) in 1,4-dioxane (5 mL) was added tert-butyl 6-bromo-2-[(2R)-1-methylpyrrolidin-2-yl]pyrrolo[3,2-c]pyridine-1-carboxylate (Intermediate 1, 128 mg, 0.337 mmol), Brettphos (108 mg, 0.202 mmol), Brettphos Pd G3 (91.6 mg, 0.101 mmol) and cesium carbonate (219 mg, 0.674 mmol). The resulting mixture was stirred at 100° C. for 16 h. The crude material was purified by combi-flash column chromatography, by eluting with 12% MeOH in DCM, to afford tert-butyl 4-[4-(6-{[1-(tert-butoxycarbonyl)-2-[(2R)-1-methylpyrrolidin-2-yl]pyrrolo[3,2-c]pyridin-6-yl]carbamoyl}-3-methylindazol-1-yl)butyl]piperazine-1-carboxylate (Intermediate 92, 150 mg, 59%) as a light yellow solid. Mass spec: m/z: 715 [M+H]⁺.

### Step 6

### Intermediate 94: 3-methyl-N-{2-[(2R)-1-methylpyrrolidin-2-yl]-1H-pyrrolo[3,2-c]pyridin-6-yl}-1-[4-(piperazin-1-yl)butyl]indazole-6-carboxamide ditrifluoroacetate

To a solution of tert-butyl 4-[4-(6-{[1-(tert-butoxycarbonyl)-2-[(2R)-1-methylpyrrolidin-2-yl]pyrrolo[3,2-c]pyridin-6-yl]carbamoyl}-3-methylindazol-1-yl)butyl]piperazine-1-carboxylate (130 mg, 0.182 mmol) in dichloromethane (1 mL) was added TFA (0.5 mL). The reaction mixture was stirred at room temperature for 1 h and concentrated in vacuo to afford 3-methyl-N-{2-[(2R)-1-methylpyrrolidin-2-yl]-1H-pyrrolo[3,2-c]pyridin-6-yl}-1-[4-(piperazin-1-yl)butyl]indazole-6-carboxamide ditrifluoroacetate (Intermediate 94, 93.0 mg, 94%) as a brown oil, which was used for the next step without further purification. Mass spec: m/z: 515 [M+H]⁺.

### Step 7

### Example 19: 1-(4-{4-[2-(2,6-dioxopiperidin-3-yl)-1,3-dioxoisoindol-5-yl]piperazin-1-yl}butyl)-3-methyl-N-{2-[(2R)-1-methylpyrrolidin-2-yl]-1H-pyrrolo[3,2-c]pyridin-6-yl}indazole-6-carboxamide

To a solution of 3-methyl-N-{2-[(2R)-1-methylpyrrolidin-2-yl]-1H-pyrrolo[3,2-c]pyridin-6-yl}-1-[4-(piperazin-1-yl)butyl]indazole-6-carboxamide ditrifluoroacetate (Intermediate 94, 93.0 mg, 0.181 mmol) in DMSO (2 mL) was added 2-(2,6-dioxopiperidin-3-yl)-5-fluoroisoindole-1,3-dione (CAS No: 835616-61-0, 49.9 mg, 0.181 mmol) and DIPEA (140 mg, 1.086 mmol) and the resulting mixture was stirred at 120° C. for 16 h. The crude product was then purified by preparative HPLC (Column: XBridge Prep OBD C18 Column, 30*150 mm, 5 μm; Mobile Phase A: Water (+10 mmol/L NH₄HCO₃), Mobile Phase B: MeCN; Flow rate: 60 mL/min mL/min; Gradient: 30% B to 60% B in 7 min) to afford 1-(4-{4-[2-(2,6-dioxopiperidin-3-yl)-1,3-dioxoisoindol-5-yl]piperazin-1-yl}butyl)-3-methyl-N-{2-[(2R)-1-methylpyrrolidin-2-yl]-1H-pyrrolo[3,2-c]pyridin-6-yl}indazole-6-carboxamide (Example 19, 22 mg, 15%) as a yellow solid. ¹H NMR (400 MHz, MeOH-d4) δ ppm 8.55 (s, 1H), 8.22 (d, J=5.6 Hz, 2H), 7.87 (d, J=8.4 Hz, 1H), 7.75 (d, J=8.8 Hz, 1H), 7.60 (d, J=8.4 Hz 1H), 7.30 (s, 1H), 7.14 (d, J=8.8 Hz 1H), 6.53 (s, 1H), 5.05-5.09 (m, 1H), 4.52 (t, J=7.2 Hz, 2H), 3.40-3.47 (m, 5H), 3.32-3.33 (m, 1H), 2.82-2.87 (m, 3H), 2.70-2.77 (m, 3H), 2.57-2.62 (m, 4H), 2.32 (s, 3H), 2.11-2.12 (m, 4H), 2.01-2.09 (m, 6H), 1.57-1.59 (m, 2H). Mass spec: m/z: 771 [M+H]⁺.

### Example 20 was Synthesized Following Scheme 19

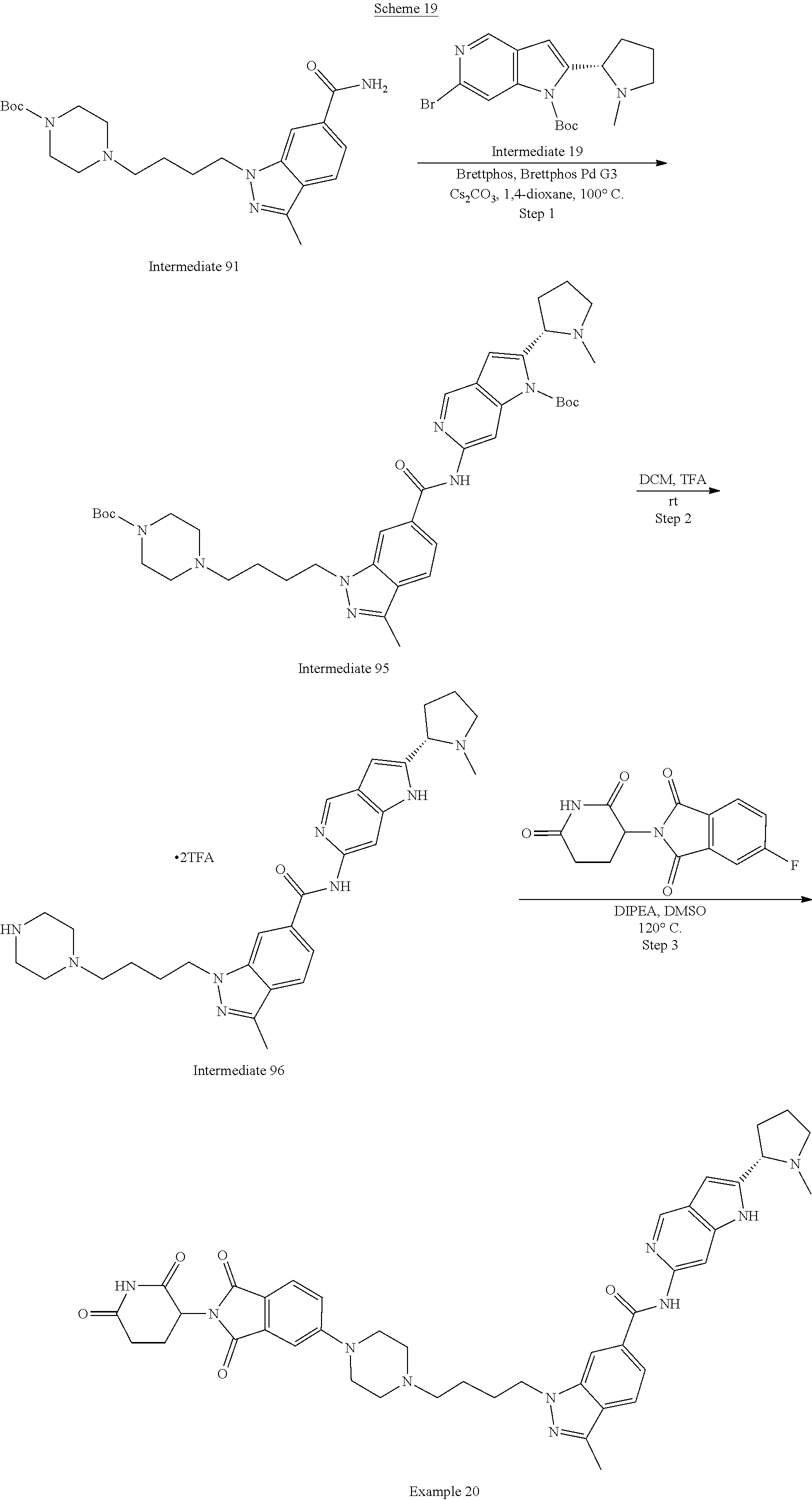 [SMILES: Cc1nn(CCCCN2CCN(C(=O)OC(C)(C)C)CC2)c2cc(C(N)=O)ccc12 ; CN1CCC[C@H]1c1cc2cnc(Br)cc2n1C(=O)OC(C)(C)C ; Cc1nn(CCCCN2CCN(C(=O)OC(C)(C)C)CC2)c2cc(C(=O)Nc3cc4c(cn3)cc([C@@H]3CCCN3C)n4C(=O)OC(C)(C)C)ccc12 ; Cc1nn(CCCCN2CCNCC2)c2cc(C(=O)Nc3cc4[nH]c([C@@H]5CCCN5C)cc4cn3)ccc12 ; O=C1CCC(N2C(=O)c3ccc(F)cc3C2=O)C(=O)N1 ; Cc1nn(CCCCN2CCN(c3ccc4c(c3)C(=O)N(C3CCC(=O)NC3=O)C4=O)CC2)c2cc(C(=O)Nc3cc4[nH]c([C@@H]5CCCN5C)cc4cn3)ccc12]

### Step 1

### Intermediate 95: tert-butyl 4-[4-(6-{[1-(tert-butoxycarbonyl)-2-[(2S)-1-methylpyrrolidin-2-yl]pyrrolo[3,2-c]pyridin-6-yl]carbamoyl}-3-methylindazol-1-yl)butyl]piperazine-1-carboxylate

Intermediate 95 was synthesised in an analogous manner to Intermediate 93, shown in Step 5 of Scheme 18, using tert-butyl 4-[4-(6-carbamoyl-3-methylindazol-1-yl)butyl]piperazine-1-carboxylate (Intermediate 91) as the amide and tert-butyl 6-bromo-2-[(2S)-1-methylpyrrolidin-2-yl]pyrrolo[3,2-c]pyridine-1-carboxylate as the aryl bromide (Intermediate 19) to afford tert-butyl 4-[4-(6-{[1-(tert-butoxycarbonyl)-2-[(2S)-1-methylpyrrolidin-2-yl]pyrrolo[3,2-c]pyridin-6-yl]carbamoyl}-3-methylindazol-1-yl)butyl]piperazine-1-carboxylate (Intermediate 95). Mass spec: m/z: 715.4 [M+H]⁺.

### Step 2

### Intermediate 96: 3-methyl-N-{2-[(2S)-1-methylpyrrolidin-2-yl]-1H-pyrrolo[3,2-c]pyridin-6-yl}-1-[4-(piperazin-1-yl)butyl]indazole-6-carboxamide ditrifluoroacetate

Intermediate 96 was synthesised in an analogous manner to Intermediate 94, shown in Step 6 of Scheme 18, using TFA as the acid to afford 3-methyl-N-{2-[(2S)-1-methylpyrrolidin-2-yl]-1H-pyrrolo[3,2-c]pyridin-6-yl}-1-[4-(piperazin-1-yl)butyl]indazole-6-carboxamide ditrifluoroacetate (Intermediate 96). Mass spec: m/z: 515.4 [M+H]⁺.

### Step 3

### Example 20: 1-(4-{4-[2-(2,6-dioxopiperidin-3-yl)-1,3-dioxoisoindol-5-yl]piperazin-1-yl}butyl)-3-methyl-N-{2-[(2S)-1-methylpyrrolidin-2-yl]-1H-pyrrolo[3,2-c]pyridin-6-yl}indazole-6-carboxamide

Example 20 was synthesised in an analogous manner to Example 19, shown in Step 7 of Scheme 18, using 3-methyl-N-{2-[(2S)-1-methylpyrrolidin-2-yl]-1H-pyrrolo[3,2-c]pyridin-6-yl}-1-[4-(piperazin-1-yl)butyl]indazole-6-carboxamide ditrifluoroacetate (Intermediate 96) as the amine and 2-(2,6-dioxopiperidin-3-yl)-5-fluoroisoindole-1,3-dione (CAS No: 835616-61-0) as the aryl fluoride, to afford 1-(4-{4-[2-(2,6-dioxopiperidin-3-yl)-1,3-dioxoisoindol-5-yl]piperazin-1-yl}butyl)-3-methyl-N-{2-[(2S)-1-methylpyrrolidin-2-yl]-1H-pyrrolo[3,2-c]pyridin-6-yl}indazole-6-carboxamide (Example 20). ¹H NMR (400 MHz, CD₃OD) δ ppm 8.54 (s, 1H), 8.24 (d, J=7.6 Hz, 2H), 7.88 (d, J=9.2 Hz, 1H), 7.75 (d, J=7.2 Hz, 1H), 7.60 (d, J=8.8 Hz, 1H), 7.32 (s, 1H), 7.12-7.15 (m, 1H), 6.53 (s, 1H), 4.51-4.54 (m, 2H), 3.63-3.65 (m, 1H), 3.49-3.51 (m, 1H), 3.40-3.46 (m, 6H), 3.25-3.27 (m, 1H), 3.14-3.15 (m, 1H), 2.65-2.71 (m, 3H), 2.56-2.61 (m, 4H), 2.40-2.48 (m, 3H), 2.25-2.28 (m, 4H), 2.02-2.11 (m, 6H), 1.57-1.59 (m, 2H). Mass spec: m/z: 771.7 [M+H]⁺.

### Example 21 was Synthesised Following Scheme 20

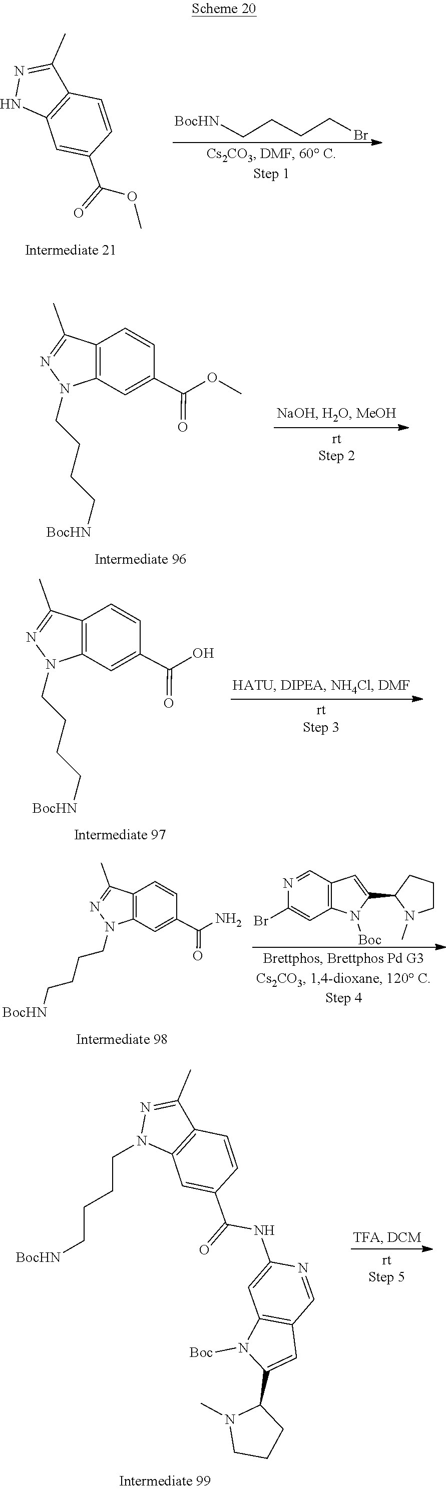

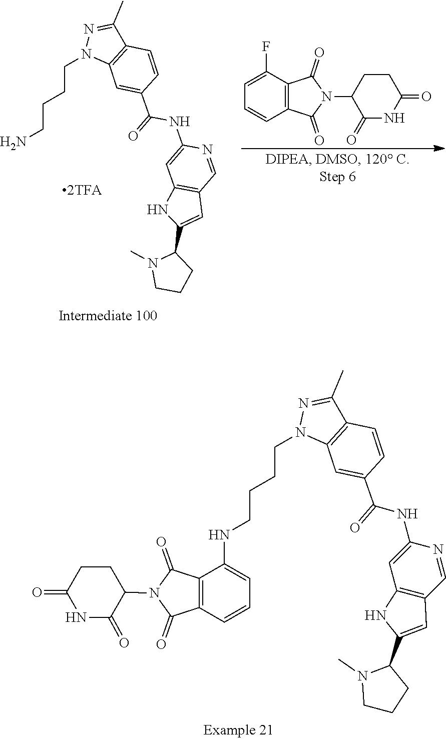 [SMILES: Cc1nn(CCCCN)c2cc(C(=O)Nc3cc4[nH]c([C@H]5CCCN5C)cc4cn3)ccc12 ; O=C1CCC(N2C(=O)c3cccc(F)c3C2=O)C(=O)N1 ; Cc1nn(CCCCNc2cccc3c2C(=O)N(C2CCC(=O)NC2=O)C3=O)c2cc(C(=O)Nc3cc4[nH]c([C@H]5CCCN5C)cc4cn3)ccc12]

### Step 1

### Intermediate 96: methyl 1-(4-((tert-butoxycarbonyl)amino)butyl)-3-methyl-1H-indazole-6-carboxylate

To a solution of methyl 3-methyl-1H-indazole-6-carboxylate (Intermediate 21, 500 mg, 2.37 mmol) in DMF (10 mL) was added tert-butyl N-(4-bromobutyl)carbamate (CAS No: 164365-88-2, 776 mg, 3.08 mmol) and Cs₂CO₃ (2.31 g, 7.10 mmol) and the reaction was stirred at 60° C. for 3h. Water (30 mL) was added and the aqueous phase was extracted with EtOAc (3×50 mL). The organic layers were combined, washed with brine (200 mL), dried over anhydrous sodium sulfate, filtered and concentrated under reduced pressure. The residue was chromatographed on a silica gel column, eluting with EtOAc/petroleum ether (20/80) to afford methyl 1-(4-((tert-butoxycarbonyl)amino)butyl)-3-methyl-1H-indazole-6-carboxylate (Intermediate 96, 550 mg, 63%) as yellow oil. Mass spec: m/z 362.2 [M+H]⁺.

### Step 2

### Intermediate 97: 1-(4-((tert-butoxycarbonyl)amino)butyl)-3-methyl-1H-indazole-6-carboxylic acid

To a solution of methyl 1-(4-((tert-butoxycarbonyl)amino)butyl)-3-methyl-1H-indazole-6-carboxylate (Intermediate 96, 550 mg, 1.49 mmol) in MeOH (10 mL) was added NaOH (800 mg, 20.0 mmol) in H₂O (10 mL) and the reaction was stirred at room temperature for 3 h. The pH of the mixture was adjusted to pH ~6 by the dropwise addition of diluted aqueous HCl (1M). The mixture was then extracted with EtOAc (3×50 mL) and the organic layers were combined, washed with brine (200 mL), dried over anhydrous sodium sulfate, filtered and concentrated under reduced pressure to afford 1-(4-((tert-butoxycarbonyl)amino)butyl)-3-methyl-1H-indazole-6-carboxylic acid (Intermediate 97, 520 mg, 98%) as a yellow solid, which was used in the next step without further purification. Mass spec: m/z 348.1 [M+H]⁺.

### Step 3

### Intermediate 98: tert-butyl (4-(6-carbamoyl-3-methyl-1H-indazol-1-yl)butyl)carbamate

To a solution of 1-(4-((tert-butoxycarbonyl)amino)butyl)-3-methyl-1H-indazole-6-carboxylic acid (Intermediate 97, 250 mg, 0.705 mmol, 98%) in DMF (5 mL) was added NH₄Cl (151 mg, 2.82 mmol), HATU (402 mg, 1.06 mmol) and DIPEA (547 mg, 4.23 mmol) and the reaction was stirred overnight at room temperature. Water (10 mL) was then added and the resulting solution was extracted with EtOAc (3×20 mL). The organic layers were combined, washed with brine (100 mL), dried over anhydrous sodium sulfate, filtered and concentrated under reduced pressure. The residue was then chromatographed on a silica gel column, eluting with MeOH/DCM (3/97) to afford tert-butyl (4-(6-carbamoyl-3-methyl-1H-indazol-1-yl)butyl)carbamate (Intermediate 98, 240 mg, 97%) as yellow oil. Mass spec: m/z 347.2 [M+H]⁺.

### Step 4

### Intermediate 99: tert-butyl (R)-6-(1-(4-((tert-butoxycarbonyl)amino)butyl)-3-methyl-1H-indazole-6-carboxamido)-2-(1-methylpyrrolidin-2-yl)-1H-pyrrolo[3,2-c]pyridine-1-carboxylate

To a solution of tert-butyl (4-(6-carbamoyl-3-methyl-1H-indazol-1-yl)butyl)carbamate (Intermediate 98, 200 mg, 0.57 mmol) in 1,4-dioxane (4 mL) was added tert-butyl (R)-6-bromo-2-(1-methylpyrrolidin-2-yl)-1H-pyrrolo[3,2-c]pyridine-1-carboxylate (Intermediate 1, 217 mg, 0.572 mmol), Brettphos (184 mg, 0.343 mmol), Brettphos Pd G₃ (155 mg, 0.172 mmol) and Cs₂CO₃ (372 mg, 1.14 mmol) and the mixture was stirred overnight at 120° C. under nitrogen. The volatiles were then concentrated under reduced pressure and the resulting residue was chromatographed on a silica gel column, eluting with MeOH/DCM (4/96), to afford tert-butyl (R)-6-(1-(4-((tert-butoxycarbonyl)amino)butyl)-3-methyl-1H-indazole-6-carboxamido)-2-(1-methylpyrrolidin-2-yl)-1H-pyrrolo[3,2-c]pyridine-1-carboxylate (Intermediate 99, 160 mg, 37%) as yellow oil. Mass spec: m/z 646.3 [M+H]⁺.

### Step 5

### Intermediate 100: (R)-1-(4-aminobutyl)-3-methyl-N-(2-(1-methylpyrrolidin-2-yl)-1H-pyrrolo[3,2-c]pyridin-6-yl)-1H-indazole-6-carboxamide ditrifluoroacetate

To a solution of tert-butyl (R)-6-(1-(4-((tert-butoxycarbonyl)amino)butyl)-3-methyl-1H-indazole-6-carboxamido)-2-(1-methylpyrrolidin-2-yl)-1H-pyrrolo[3,2-c]pyridine-1-carboxylate (Intermediate 99, 160 mg, 0.21 mmol) in DCM (2 mL) was added TFA (1 mL) and the reaction was stirred at room temperature for 2 h. The volatiles were then removed under reduced pressure to afford (R)-1-(4-aminobutyl)-3-methyl-N-(2-(1-methylpyrrolidin-2-yl)-1H-pyrrolo[3,2-c]pyridin-6-yl)-1H-indazole-6-carboxamide ditrifluoroacetate (Intermediate 100, 110 mg, 93%) as yellow oil, which was used in the next step without further purification. Mass spec: m/z 446.2 [M+H]⁺.

### Step 6

### Example 21: 1-(4-((2-(2,6-dioxopiperidin-3-yl)-1,3-dioxoisoindolin-4-yl)amino)butyl)-3-methyl-N-(2-((R)-1-methylpyrrolidin-2-yl)-1H-pyrrolo[3,2-c]pyridin-6-yl)-1H-indazole-6-carboxamide

To a solution of (R)-1-(4-aminobutyl)-3-methyl-N-(2-(1-methylpyrrolidin-2-yl)-1H-pyrrolo[3,2-c]pyridin-6-yl)-1H-indazole-6-carboxamide ditrifluoroacetate (Intermediate 100, 110 mg, 0.197 mmol) in DMSO (4 mL) was added 2-(2,6-dioxopiperidin-3-yl)-4-fluoroisoindoline-1,3-dione (CAS No: 835616-60-9, 65.5 mg, 0.236 mmol) and DIPEA (76.6 mg, 0.591 mmol) and the reaction mixture was stirred overnight at 120° C. Water (10 mL) was added and resulting mixture was purified by preparative SFC (Column: DAICEL DCpak P4VP 3*25 cm, 5 um; Mobile Phase A: CO₂, Mobile Phase B: MeOH (1% 2M NH₃ in MeOH); Flow rate: 65 mL/min; Gradient: isocratic 46% B) to afford 1-(4-((2-(2,6-dioxopiperidin-3-yl)-1,3-dioxoisoindolin-4-yl)amino)butyl)-3-methyl-N-(2-((R)-1-methylpyrrolidin-2-yl)-1H-pyrrolo[3,2-c]pyridin-6-yl)-1H-indazole-6-carboxamide (Example 21, 16.8 mg, 12%) as a yellow solid. ¹H NMR (400 MHz, DMSO-d6) δ ppm 11.41 (s, 1H), 11.08 (s, 1H), 10.54 (s, 1H), 8.48 (s, 1H), 8.53 (s, 1H), 8.26 (s, 1H), 7.73-7.81 (m, 2H), 7.54 (t, J=7.2 Hz, 1H), 7.08 (d, J=8.4 Hz, 1H), 7.00 (d, J=6.8 Hz, 1H), 6.61 (t, J=5.6 Hz, 1H), 6.41 (s, 1H), 5.01-5.05 (m, 1H), 4.47 (t, J=6.8 Hz, 2H), 3.12-3.20 (m, 2H), 2.83-2.87 (m, 1H), 2.50-2.59 (m, 5H), 2.24-2.29 (m, 1H), 2.15-2.19 (m, 4H), 1.79-2.02 (m, 7H), 1.56-1.60 (m, 3H). Mass spec: m/z 702.4 [M+H]⁺.

### Example 22 was Synthesised Following Scheme 21

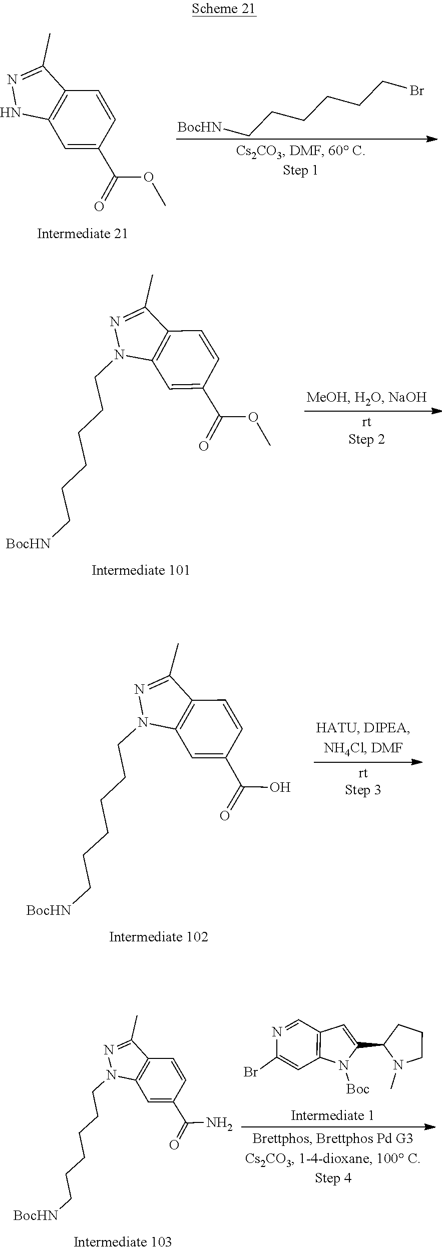 [SMILES: COC(=O)c1ccc2c(C)n[nH]c2c1 ; CC(C)(C)OC(=O)NCCCCCCBr ; COC(=O)c1ccc2c(C)nn(CCCCCCNC(=O)OC(C)(C)C)c2c1 ; Cc1nn(CCCCCCNC(=O)OC(C)(C)C)c2cc(C(=O)O)ccc12 ; Cc1nn(CCCCCCNC(=O)OC(C)(C)C)c2cc(C(N)=O)ccc12 ; CN1CCC[C@@H]1c1cc2cnc(Br)cc2n1C(=O)OC(C)(C)C]

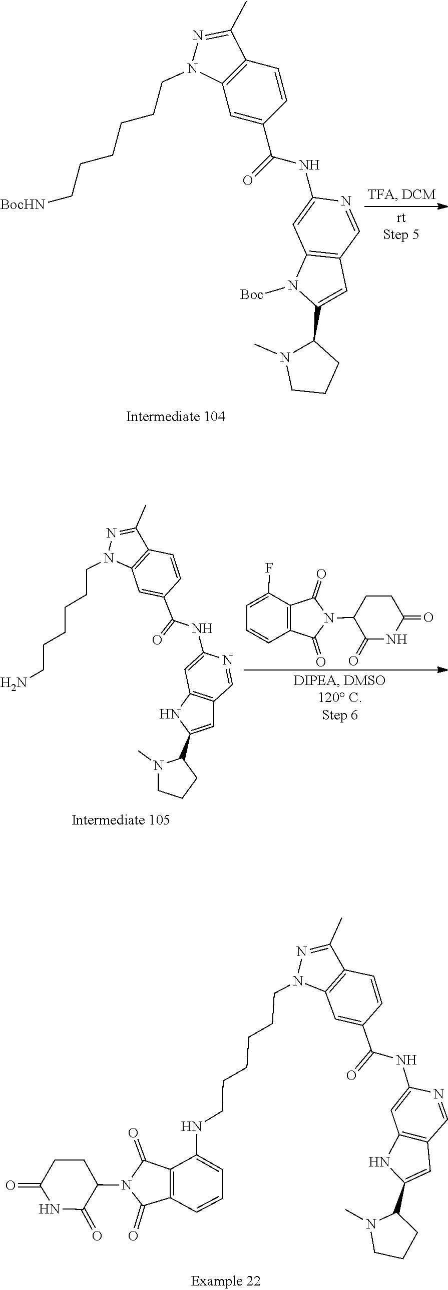

### Step 1

### Intermediate 101: methyl 1-{6-[(tert-butoxycarbonyl)amino]hexyl}-3-methylindazole-6-carboxylate

To a solution of methyl 3-methyl-1H-indazole-6-carboxylate (Intermediate 21, 500 mg, 2.62 mmol) in DMF (20 mL) was added tert-butyl N-(6-bromohexyl)carbamate (CAS No: 142356-33-0, 810 mg, 2.89 mmol) and Cs₂CO₃ (2.6 g, 7.88 mmol) and the reaction was stirred overnight at 60° C. for 3 h. The reaction was quenched with water (300 mL) and extracted with EtOAc (3×300 mL). The combined organic layers were washed with brine, dried over anhydrous Na₂SO₄ and concentrated under reduced pressure. The residue was chromatographed on a silica gel column with EtOAc/petroleum ether (30/70) to afford methyl 1-{6-[(tert-butoxycarbonyl)amino]hexyl}-3-methylindazole-6-carboxylate (Intermediate 101, 800 mg, 75%) as a yellow solid. Mass spec: m/z: 390.0 [M+H]⁺.

### Step 2

### Intermediate 102: 1-{6-[(tert-butoxycarbonyl)amino]hexyl}-3-methylindazole-6-carboxylic acid

To a solution of methyl 1-{6-[(tert-butoxycarbonyl)amino]hexyl}-3-methylindazole-6-carboxylate (Intermediate 101, 300 mg, 0.77 mmol) in MeOH (4 mL) was added H₂O (4 mL) and NaOH (184 mg, 4.62 mmol) and the reaction was stirred at rt for 3 h. The pH of the reaction mixture was then adjusted to pH ~5 by the drop-wise addition concentrated aqueous HCl. The resulting mixture was extracted with EtOAc (3×50 mL), the organic phases were dried over anhydrous sodium sulfate and concentrated under reduced pressure to afford 1-{6-[(tert-butoxycarbonyl)amino]hexyl}-3-methylindazole-6-carboxylic acid (Intermediate 102, 270 mg, 92%) as a yellow solid, which was used in the next step without further purification. Mass spec: m/z: 376.2 [M+H]⁺.

### Step 3

### Intermediate 103: tert-butyl N-[6-(6-carbamoyl-3-methylindazol-1-yl)hexyl]carbamate

To a solution of 1-{6-[(tert-butoxycarbonyl)amino]hexyl}-3-methylindazole-6-carboxylic acid (Intermediate 102, 270 mg, 0.719 mmol) in DMF (8 mL) was added NH₄Cl (153 mg, 2.87 mmol) and DIPEA (557 mg, 4.31 mmol), HATU (410 mg, 1.07 mmol) and the reaction was stirred overnight at rt. The mixture was then quenched with water (200 mL) and the aqueous phase extracted with EtOAc (3×200 mL). The combined organic layers were washed with brine, dried over anhydrous Na₂SO₄, and the volatiles were removed under reduced pressure. The crude was then chromatographed on a silica gel column with MeOH/DCM (3/97) to afford tert-butyl N-[6-(6-carbamoyl-3-methylindazol-1-yl)hexyl]carbamate (Intermediate 103, 240 mg, 88%) as a yellow solid. Mass spec: m/z: 375.2 [M+H]⁺.

### Step 4

### Intermediate 104: tert-butyl 6-(1-{6-[(tert-butoxycarbonyl)amino]hexyl}-3-methylindazole-6-amido)-2-[(2R)-1-methylpyrrolidin-2-yl]pyrrolo[3,2-c]pyridine-1-carboxylate

To a solution of tert-butyl N-[6-(6-carbamoyl-3-methylindazol-1-yl)hexyl]carbamate (Intermediate 103, 200 mg, 0.53 mmol) in 1,4-dioxane (7 mL) was added tert-butyl 6-bromo-2-[(2R)-1-methylpyrrolidin-2-yl]pyrrolo[3,2-c]pyridine-1-carboxylate (Intermediate 1, 203 mg, 0.53 mmol), Brettphos (172 mg, 0.320 mmol), Brettphos Pd G3 (145 mg, 0.160 mmol) and Cs₂CO₃ (348 mg, 1.06 mmol) and the resulting mixture was stirred overnight at 100° C. under N₂. The reaction mixture was then purified on a silica gel column, eluting with MeOH/DCM (6/94), to afford tert-butyl 6-(1-{6-[(tert-butoxycarbonyl)amino]hexyl}-3-methylindazole-6-amido)-2-[(2R)-1-methylpyrrolidin-2-yl]pyrrolo[3,2-c]pyridine-1-carboxylate (Intermediate 104, 178 mg, 47%) as a yellow solid. Mass spec: m/z: 674.3 [M+H]⁺.

### Step 5

### Intermediate 105: 1-(6-aminohexyl)-3-methyl-N-{2-[(2R)-1-methylpyrrolidin-2-yl]-1H-pyrrolo[3,2-c]pyridin-6-yl}indazole-6-carboxamide ditrifluoroacetate

To a solution of tert-butyl 6-(1-{6-[(tert-butoxycarbonyl)amino]hexyl}-3-methylindazole-6-amido)-2-[(2R)-1-methylpyrrolidin-2-yl]pyrrolo[3,2-c]pyridine-1-carboxylate (Intermediate 104, 178 mg, 0.251 mmol) in DCM (6 mL) was added TFA (3 mL) and the reaction mixture was stirred at rt for 2 h. The volatiles were then removed under reduced pressure to afford 1-(6-aminohexyl)-3-methyl-N-{2-[(2R)-1-methylpyrrolidin-2-yl]-1H-pyrrolo[3,2-c]pyridin-6-yl}indazole-6-carboxamide ditrifluoroacetate (Intermediate 105, 125 mg, 94%) as a yellow oil, which was used in the next step without further purification. Mass spec m/z: 474.3 [M+H]⁺.

### Step 6

### Example 22: 1-(6-{[2-(2,6-dioxopiperidin-3-yl)-1,3-dioxoisoindol-4-yl]amino}hexyl)-3-methyl-N-{2-[(2R)-1-methylpyrrolidin-2-yl]-1H-pyrrolo[3,2-c]pyridin-6-yl}indazole-6-carboxamide

To a solution of 1-(6-aminohexyl)-3-methyl-N-{2-[(2R)-1-methylpyrrolidin-2-yl]-1H-pyrrolo[3,2-c]pyridin-6-yl}indazole-6-carboxamide (Intermediate 105, 125 mg, 0.264 mmol) in DMSO (2 mL) was added 2-(2,6-dioxopiperidin-3-yl)-4-fluoroisoindole-1,3-dione (CAS No: 835616-60-9, 72.9 mg, 0.264 mmol) and DIPEA (102 mg, 0.792 mmol) and the reaction stirred overnight at 120° C. The crude product was then purified by preparative HPLC (Column: XBridge Prep OBD C18 Column, 30*150 mm, 5 μm; Mobile Phase A: Water (10 mmol/L NH₄HCO₃), Mobile Phase B: MeCN; Flow rate: 60 mL/min mL/min; Gradient: 38% B to 68% B in 10 min) to afford 1-(6-{[2-(2,6-dioxopiperidin-3-yl)-1,3-dioxoisoindol-4-yl]amino}hexyl)-3-methyl-N-{2-[(2R)-1-methylpyrrolidin-2-yl]-1H-pyrrolo[3,2-c]pyridin-6-yl}indazole-6-carboxamide (Example 22, 5.7 mg, 3%) as a yellow solid. ¹H NMR (400 MHz, DMSO-d6) δ ppm 8.55 (s, 1H), 8.22 (d, J=9.2 Hz 2H), 7.84 (d, J=8.4 Hz, 1H), 7.74 (d, J=7.6 Hz 1H), 7.49 (t, J=7.2 Hz 1H), 6.95-7.00 (m, 2H), 6.53 (s, 1H), 5.01-5.04 (m, 1H), 4.46 (t, J=6.8 Hz, 2H), 3.44-3.45 (m, 1H), 3.32-3.33 (m, 3H), 2.70-2.74 (m, 3H), 2.59 (s, 3H), 2.40-2.43 (m, 1H), 2.32 (s, 4H), 2.04-2.09 (m, 3H), 1.94-1.99 (m, 3H), 1.61-1.65 (m, 2H), 1.45-1.49 (m, 2H), 1.30-1.38 (m, 2H). Mass spec: m/z: 730.5 [M+H]⁺.

### Example 23 was Synthesized Following Scheme 22

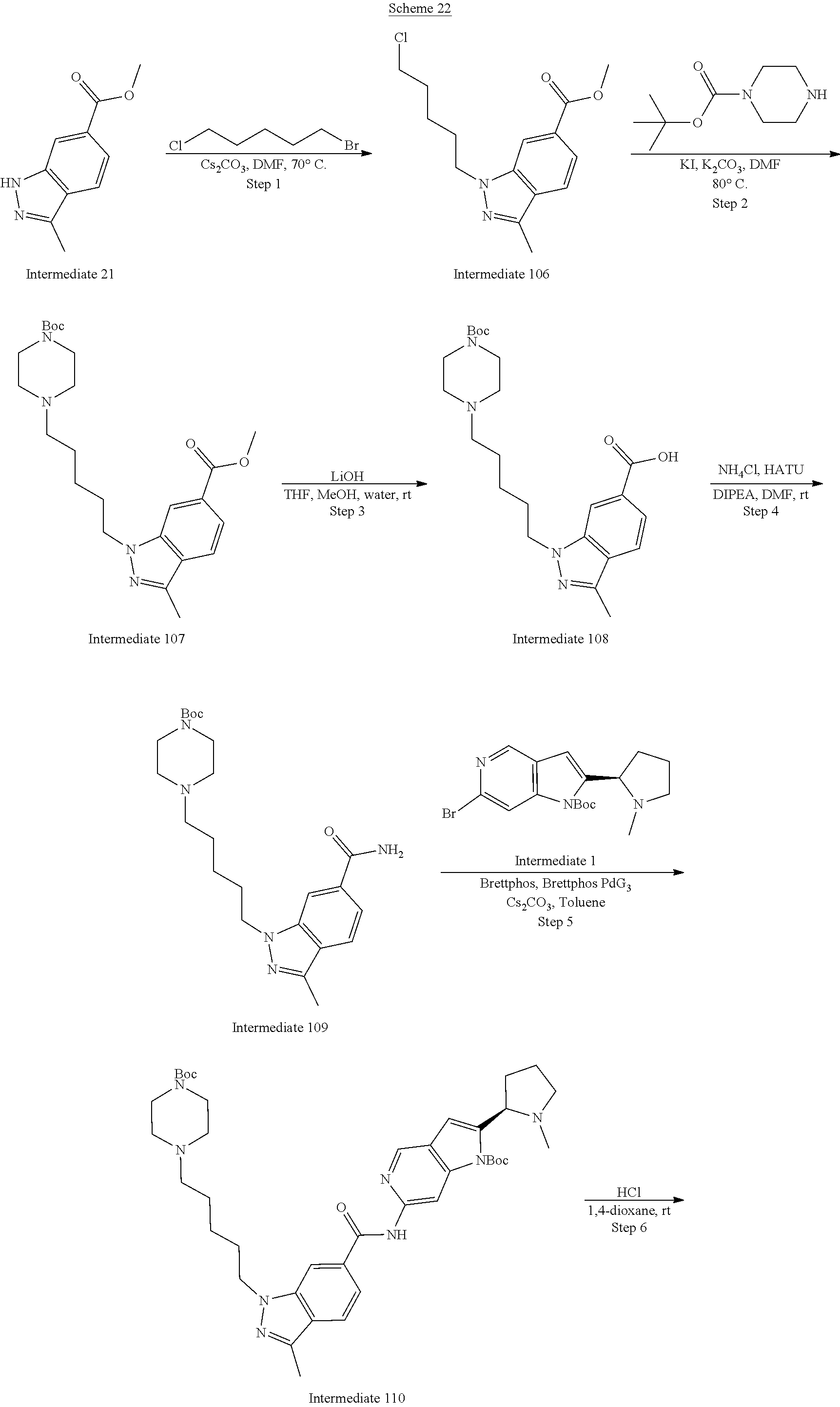

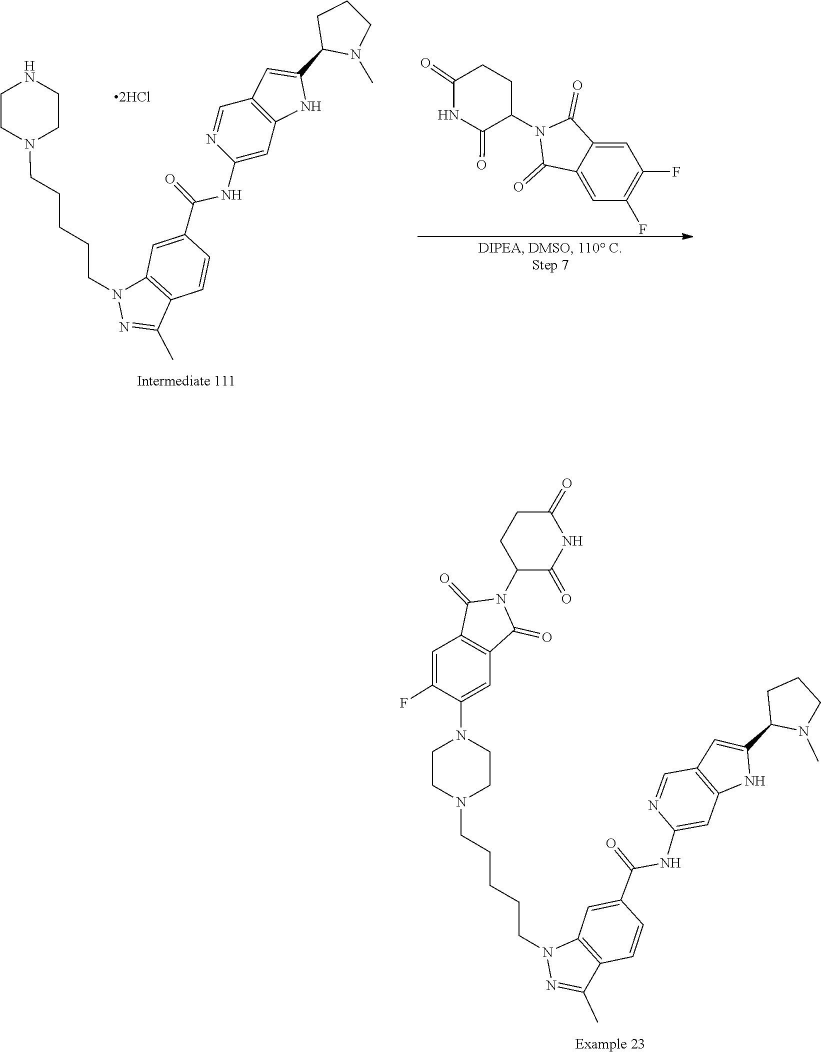 [SMILES: Cc1nn(CCCCCN2CCNCC2)c2cc(C(=O)Nc3cc4[nH]c([C@H]5CCCN5C)cc4cn3)ccc12 ; O=C1CCC(N2C(=O)c3cc(F)c(F)cc3C2=O)C(=O)N1 ; Cc1nn(CCCCCN2CCN(c3cc4c(cc3F)C(=O)N(C3CCC(=O)NC3=O)C4=O)CC2)c2cc(C(=O)Nc3cc4[nH]c([C@H]5CCCN5C)cc4cn3)ccc12]

### Step 1

### Intermediate 106: Methyl 1-(5-chloropentyl)-3-methyl-1H-indazole-6-carboxylate

To a solution of methyl 3-methyl-1H-indazole-6-carboxylate (Intermediate 21, 3.0 g, 15.8 mmol) in DMF (30 mL) was added cesium carbonate (12.9 g, 39.4 mmol) followed by 1-bromo-5-chloropentane (CAS No: 54512-75-3, 5.97 g, 31.6 mmol). The reaction mixture was heated to 70° C. for 16 h. The reaction mixture was cooled to room temperature, water added (100 mL) and the mixture extracted with ethyl acetate (3×60 mL). The combined organic layers were dried, washed with brine solution (2×50 mL), dried over Na₂SO₄ and concentrated in vacuo. The obtained residue was purified by combi-flash column chromatography, by eluting with 10% ethyl acetate in heptane, to afford methyl 1-(5-chloropentyl)-3-methyl-1H-indazole-6-carboxylate (Intermediate 106, 3.2 g, 69%) as a yellow liquid. 1H NMR (400 MHz, DMSO-d6) δ ppm 1.32-1.41 (m, 2H), 1.69-1.87 (m, 4H), 2.51 (s, 3H), 3.59 (t, J=6.47 Hz, 2H), 3.90 (s, 3H), 4.42 (t, J=6.94 Hz, 2H), 7.65 (d, J=8.32 Hz, 1H), 7.81 (d, J=8.32 Hz, 1H), 8.25 (s, 1H). Mass spec: m/z 294.8 [M+H]⁺.

### Step 2

### Intermediate 107: Methyl 1-(5-(4-(tert-butoxycarbonyl)piperazin-1-yl)pentyl)-3-methyl-1H-indazole-6-carboxylate

To a solution of methyl 1-(5-chloropentyl)-3-methyl-1H-indazole-6-carboxylate (Intermediate 106, 3.3 g, 11 mmol) in dimethylformamide (35 mL) was added potassium carbonate (3.1 g, 22 mmol), potassium iodide (1.9 g, 11 mmol) and tert-butyl piperazine-1-carboxylate (CAS No: 57260-71-6, 2.6 g, 13 mmol). The reaction mixture was heated to 80° C. for 16 h. The reaction mixture cooled to room temperature, treated with water (100 mL) and extracted with EtOAc (3×100 mL). The organic layers were washed with brine solution (3×50 mL), dried over Na₂SO₄, and concentrated in vacuo to afford methyl 1-(5-(4-(tert-butoxycarbonyl)piperazin-1-yl)pentyl)-3-methyl-1H-indazole-6-carboxylate (Intermediate 107, 3.85 g, 77%) a yellow liquid. ¹H NMR (400 MHz, DMSO-d6) δ ppm 1.20-1.22 (m, 2H), 1.36 (s, 9H) 1.37-1.45 (m, 4H), 1.76-1.86 (m, 2H), 2.15-2.27 (m, 2H), 2.51 (s, 3H), 2.58-2.63 (m, 1H), 3.23-3.25 (m, 5H), 3.90 (s, 3H), 4.40 (t, J=6.80 Hz, 2H), 7.65 (d, J=8.33 Hz, 1H), 7.81 (d, J=8.33 Hz, 1H) 8.23 (s, 1H). Mass spec: m/z 445.47 [M+H]⁺.

### Step 3

### Intermediate 108: 1-(5-(4-(tert-butoxycarbonyl)piperazin-1-yl)pentyl)-3-methyl-1H-indazole-6-carboxylic acid

To a solution of methyl 1-(5-(4-(tert-butoxycarbonyl)piperazin-1-yl)pentyl)-3-methyl-1H-indazole-6-carboxylate (Intermediate 107, 3.4 g, 7.6 mmol) in THF (12 mL) and MeOH (12 mL) was added LiOH (0.47 g, 19 mmol) in water (12 mL). The reaction mixture was stirred at room temperature for 16 h then concentrated in vacuo. The aqueous layer pH was adjusted to 3-4 with 0.5 N HCl and extracted with ethyl acetate (2×50 mL). The combined organic layers were dried over Na₂SO₄ and concentrated in vacuo to afford 1-(5-(4-(tert-butoxycarbonyl)piperazin-1-yl)pentyl)-3-methyl-1H-indazole-6-carboxylic acid (Intermediate 108, 2.55 g, 77%) as an off-white solid which was used for the next step without further purification. ¹H NMR (400 MHz, DMSO-d6) δ ppm 1.20. 1.22 (m, 4H), 1.32 (s, 9H) 1.36-1.44 (m, 4H), 1.78-1.79 (m, 2H), 2.23-2.25 (m, 6H) 2.50 (s, 3H) 4.37-4.38 (m, 2H), 7.62 (d, J=8.32 Hz, 1H), 7.72-7.80 (m, 1H), 8.18 (s, 1H). Mass spec: m/z 431.0 [M+H]⁺.

### Step 4

### Intermediate 109: tert-Butyl 4-(5-(6-carbamoyl-3-methyl-1H-indazol-1-yl)pentyl)piperazine-1-carboxylate

To a solution of 1-(5-(4-(tert-butoxycarbonyl)piperazin-1-yl)pentyl)-3-methyl-1H-indazole-6-carboxylic acid (Intermediate 108, 1.5 g, 3.5 mmol) in dimethylformamide (15 mL) was added HATU (2.7 g, 7.0 mmol). The reaction mixture was stirred at room temperature for 15 min. Ammonium chloride (1.3 g, 24 mmol), followed by DIPEA (2.1 mL, 12 mmol) were then added and the mixture stirred at room temperature for 16 h. The reaction mixture was diluted with water (100 mL) and extracted with ethyl acetate (3×50 mL). The combined organic layers were washed sequentially with NaHCO₃ (2×40 mL) and brine solution (2×30 mL), then dried over Na₂SO₄ and concentrated in vacuo. The crude solid was washed with DCM and n-pentane to afford tert-butyl 4-(5-(6-carbamoyl-3-methyl-1H-indazol-1-yl)pentyl)piperazine-1-carboxylate (Intermediate 109, 0.94 g, 2.188 mmol, 63%) as an off-white solid which was used for the next step without further purification. Mass spec: m/z 430.5 [M+H]⁺.

### Step 5

### Intermediate 110: tert-Butyl (R)-6-(1-(5-(4-(tert-butoxycarbonyl)piperazin-1-yl)pentyl)-3-methyl-1H-indazole-6-carboxamido)-2-(1-methylpyrrolidin-2-yl)-1H-pyrrolo[3,2-c]pyridine-1-carboxylate

To a solution of tert-butyl 4-(5-(6-carbamoyl-3-methyl-1H-indazol-1-yl)pentyl)piperazine-1-carboxylate (Intermediate 109, 0.38 g, 0.89 mmol) in toluene (5 mL) was added tert-butyl 6-bromo-2-[(2R)-1-methylpyrrolidin-2-yl]pyrrolo[3,2-c]pyridine-1-carboxylate (Intermediate 1, 0.40 g, 1.06 mmol) followed by Cs₂CO₃ (0.865 g, 2.65 mmol). The reaction mixture was purged with argon for 10 min, then BrettPhos (0.073 g, 0.13 mmol) and BrettPhos Pd G3 (0.12 g, 0.13 mmol) were added. The reaction was purged with argon for 10 min and heated to 100° C. for 2h. The reaction mixture was then cooled to room temperature, treated with water (50 mL) and extracted with ethyl acetate (3×50 mL). The combined organic layers were dried over Na₂SO₄ and concentrated in vacuo. The resulting residue was purified by combi-flash column chromatography, by eluting with 1.5% MeOH in DCM, to afford tert-butyl (R)-6-(1-(5-(4-(tert-butoxycarbonyl)piperazin-1-yl)pentyl)-3-methyl-1H-indazole-6-carboxamido)-2-(1-methylpyrrolidin-2-yl)-1H-pyrrolo[3,2-c]pyridine-1-carboxylate (Intermediate 110, 0.32 g, 50%) as an off-white solid. ¹H NMR (400 MHz, DMSO-d6) δ ppm 1.19-1.29 (m, 3H), 1.35 (s, 9H), 1.42-1.49 (m, 2H), 1.60-1.63 (m, 1H), 1.71 (s, 9H), 1.86-1.88 (m, 3H), 2.16-2.27 (m, 5H), 2.39 (s, 3H) 2.40-2.41 (m, 3H), 2.52 (s, 3H), 3.10-3.17 (m, 1H), 3.20-3.22 (m, 4H), 3.89-3.97 (m, 1H), 4.41 (t, J=6.80 Hz, 2H), 6.77 (s, 1H), 7.71-7.82 (m, 2H), 8.45 (s, 1H), 8.62 (s, 1H), 8.99 (s, 1H), 10.77 (s, 1H). Mass spec: m/z 729.4 [M+H]⁺.

### Step 6

### Intermediate 111: (R)-3-methyl-N-(2-(1-methylpyrrolidin-2-yl)-1H-pyrrolo[3,2-c]pyridin-6-yl)-1-(5-(piperazin-1-yl)pentyl)-1H-indazole-6-carboxamide dihydrochloride

To a cooled (0° C.) solution of tert-butyl (R)-6-(1-(5-(4-(tert-butoxycarbonyl)piperazin-1-yl)pentyl)-3-methyl-1H-indazole-6-carboxamido)-2-(1-methylpyrrolidin-2-yl)-1H-pyrrolo[3,2-c]pyridine-1-carboxylate (Intermediate 110, 0.3 g, 0.41 mmol) in 1,4-dioxane (2 mL) was added 4M HCl in 1,4-dioxane (5 mL) and the reaction mixture was stirred at room temperature for 16 h then concentrated in vacuo. The resultant precipitate was collected by filtration, washed with dichloromethane (2×20 mL) and dried in vacuo to afford (R)-3-methyl-N-(2-(1-methylpyrrolidin-2-yl)-1H-pyrrolo[3,2-c]pyridin-6-yl)-1-(5-(piperazin-1-yl)pentyl)-1H-indazole-6-carboxamide dihydrochloride (Intermediate 111, 0.215 g, 99%) as a white solid, which was used for next step without further purification. ¹H NMR (400 MHz, DMSO-d6) δ ppm 1.28-1.39 (m, 2H), 1.75-1.77 (m, 2H), 1.88-1.97 (m, 2H), 2.16-2.24 (m, 2H), 2.55 (s, 3H), 2.82 (s, 3H), 3.06-3.16 (m, 2H), 3.33-3.54 (m, 8H), 3.58-3.81 (m, 5H), 4.48 (t, J=6.36 Hz, 2H), 7.29 (s, 1H), 7.82 (d, J=8.31 Hz, 1H), 7.88-7.93 (m, 1H), 8.45 (s, 1H), 8.87 (s, 1H), 9.12 (s, 1H), 9.71 (s, 1H), 9.70 (s, 1H), 11.70 (s, 2H). Mass spec: m/z 529.3 [M+H]⁺.

### Step 7

### Example 23: 1-(5-(4-(2-(2,6-dioxopiperidin-3-yl)-6-fluoro-1,3-dioxoisoindolin-5-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide

To a solution of (R)-3-methyl-N-(2-(1-methylpyrrolidin-2-yl)-1H-pyrrolo[3,2-c]pyridin-6-yl)-1-(5-(piperazin-1-yl)pentyl)-1H-indazole-6-carboxamide (Intermediate 111, 0.215 g, 0.4067 mmol) in DMSO (3 mL) was added 2-(2,6-dioxopiperidin-3-yl)-5,6-difluoroisoindoline-1,3-dione (CAS No: 1496997-41-1: 0.13 g, 0.45 mmol) followed by DIPEA (0.21 mL, 1.22 mmol) and the reaction mixture was heated at 110° C. for 6 h. The reaction mixture was then cooled to room temperature and diluted with water (50 mL). The resultant precipitate was collected by filtration, washed with DCM/Pentane (2×20 mL) and then dried in vacuo. The crude compound was purified by preparative HPLC (Method A) to afford 1-(5-(4-(2-(2,6-dioxopiperidin-3-yl)-6-fluoro-1,3-dioxoisoindolin-5-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide (Example 23, 0.085 g, 26%) as a yellow solid. ¹H NMR (400 MHz, DMSO-d6) δ ppm 1.24-1.33 (m, 2H), 1.46-1.55 (m, 2H), 1.77-1.94 (m, 5H), 2.00-2.08 (m, 2H), 2.16 (s, 3H), 2.23-2.32 (m, 3H), 2.44-2.48 (m, 4H), 2.52 (s, 3H), 2.54-2.59 (m, 1H), 2.60-2.69 (m, 1H), 2.86-2.90 (m, 1H), 3.16-3.17 (s, 4H), 3.26-3.29 (m, 2H), 4.43-4.46 (m, 2H), 5.10-5.13 (m 1H), 6.39-6-41 (m, 1H), 7.39 (d, J=7.38 Hz, 1H), 7.67 (d, J=11.38 Hz, 1H), 7.72-7.75 (m, 1H), 7.78-7.81 (m, 1H), 8.24 (s, 1H), 8.46 (s, 1H), 8.51 (s, 1H), 10.53 (s, 1H), 11.00-11.17 (m, 1H), 11.36 (s, 1H). Mass spec: m/z 803.3 [M+H]⁺.

### Example 24 was Synthesized Following Scheme 23

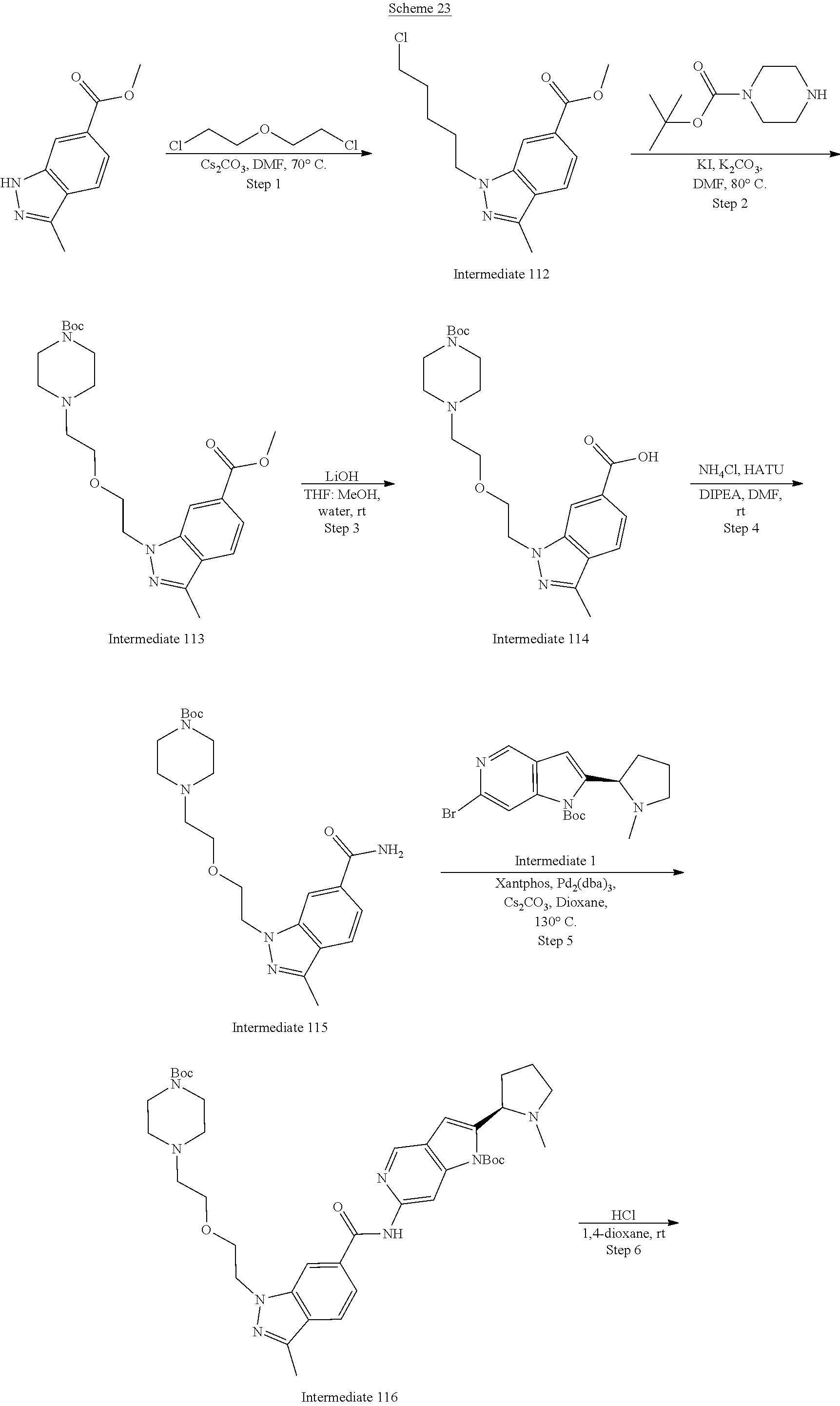

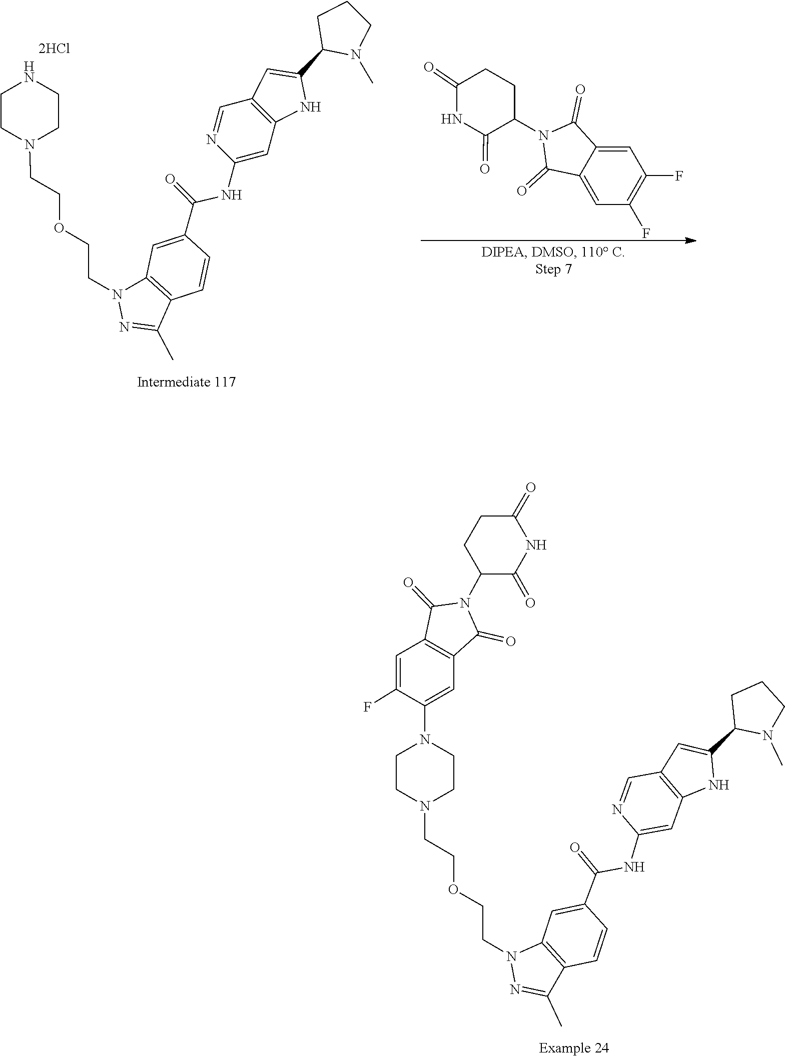 [SMILES: Cc1nn(CCOCCN2CCNCC2)c2cc(C(=O)Nc3cc4[nH]c([C@H]5CCCN5C)cc4cn3)ccc12 ; O=C1CCC(N2C(=O)c3cc(F)c(F)cc3C2=O)C(=O)N1 ; Cc1nn(CCOCCN2CCN(c3cc4c(cc3F)C(=O)N(C3CCC(=O)NC3=O)C4=O)CC2)c2cc(C(=O)Nc3cc4[nH]c([C@H]5CCCN5C)cc4cn3)ccc12]

### Step 1

### Intermediate 112: Methyl 1-(2-(2-chloroethoxy) ethyl)-3-methyl-1H-indazole-6-carboxylate

To a solution of methyl 3-methyl-1H-indazole-6-carboxylate (Intermediate 21, 3.0 g, 15.73 mmol) in DMF (20 mL) was added cesium carbonate (12.9 g, 39.43 mmol) followed by 1-chloro-2-(2-chloroethoxy)ethane (CAS No: 111-44-4, 4.55 g, 31.54 mmol) and the reaction mixture was heated at 70° C. for 16 h. The reaction mixture was diluted with water (200 mL) and extracted with ethyl acetate (3×200 mL). The combined organic layers were separated, dried over anhydrous Na₂SO₄ and concentrated in vacuo to obtain crude compound as a mixture of regioisomers. The regioisomers were separated by combi-flash column chromatography, by eluting with 30% EtOAc in heptane, to afford desired regioisomer methyl 1-(2-(2-chloroethoxy) ethyl)-3-methyl-1H-indazole-6-carboxylate (Intermediate 112, 2.6 g, 56%) as an off-white solid. ¹H NMR (400 MHz, DMSO-d₆) δ ppm 2.51 (s, 3H), 3.57-3.61 (m, 4H), 3.84-3.87 (t, J=5.04 Hz, 2H), 3.89 (s, 3H), 4.58 (t, J=5.26 Hz, 2H), 7.64 (d, J=8.33 Hz, 1H), 7.79 (d, J=8.33 Hz, 1H), 8.27 (s, 1H).

### Step 2

### Intermediate 113: methyl 1-(2-(2-(4-(tert-butoxycarbonyl) piperazin-1-yl)ethoxy)ethyl)-3-methyl-1H-indazole-6-carboxylate

To a solution of methyl 1-[2-(2-chloroethoxy)ethyl]-3-methyl-indazole-6-carboxylate (Intermediate 112, 2.4 g, 8.1 mmol) in dimethylformamide (25 mL) was added potassium carbonate (1.7 g, 12 mmol) followed by potassium iodide (1.3 g, 8.1 mmol) and tert-butyl piperazine-1-carboxylate (CAS No: 57260-71-6, 2.0 g, 9.7 mmol). The reaction mixture was heated at 80° C. for 12 h then diluted with water (200 mL) and extracted with ethyl acetate (3×200 mL). The combined organic layers were separated, dried over anhydrous Na₂SO₄ and concentrated in vacuo. The crude material was purified by combi-flash column chromatography, by eluting with 10% ethyl acetate in heptane, to afford methyl 1-(2-(2-(4-(tert-butoxycarbonyl) piperazin-1-yl)ethoxy)ethyl)-3-methyl-1H-indazole-6-carboxylate (Intermediate 113, 2.2 g, 61%) as a white solid. ¹H NMR (400 MHz, DMSO-d₆) δ ppm 1.38 (s, 9H), 2.02-2.12 (m, 4H), 2.30 (t, J=5.14 Hz, 2H), 2.51 (s, 3H), 3.04-3.11 (m, 4H), 3.42 (t, J=5.14 Hz, 2H), 3.75 (t, J=4.65 Hz, 2H), 3.90 (s, 3H), 4.57 (t, J=4.65 Hz, 2H), 7.65 (d, J=8.31 Hz, 1H), 7.79 (d, J=8.31 Hz, 1H), 8.25 (s, 1H). Mass spec: m/z 447.8 [M+H+2]⁺.

### Step 3

### Intermediate 114: 1-(2-(2-(4-(tert-butoxycarbonyl)piperazin-1-yl)ethoxy)ethyl)-3-methyl-1H-indazole-6-carboxylic acid

To a cooled (0° C.) solution of methyl 1-(2-(2-(4-(tert-butoxycarbonyl)piperazin-1-yl)ethoxy)ethyl)-3-methyl-1H-indazole-6-carboxylate (Intermediate 113, 2.2 g, 4.5 mmol) in tetrahydrofuran (10 mL) and methanol (5 mL) was added lithium hydroxide (0.33 g, 14 mmol) in water (2 mL). The reaction mixture was stirred at room temperature for 24 h then concentrated in vacuo. The residue was diluted with water (50 mL) and extracted with ethyl acetate (3×50 mL). The combined organic layers were separated, dried over anhydrous Na₂SO₄ and concentrated in vacuo. The crude material was purified by combi-flash column chromatography, by eluting with 50% ethyl acetate in heptane, to afford 1-(2-(2-(4-(tert-butoxycarbonyl) piperazin-1-yl) ethoxy) ethyl)-3-methyl-1H-indazole-6-carboxylic acid (Intermediate 114, 1.7 g, 85%) as a yellow liquid. Mass spec: m/z 433.8 [M+H]⁺.

### Step 4

### Intermediate 115: tert-butyl 4-(2-(2-(6-carbamoyl-3-methyl-1H-indazol-1-yl) ethoxy) ethyl) piperazine-1-carboxylate

To a solution of 1-[2-[2-(4-tert-butoxycarbonylpiperazin-1-yl) ethoxy]ethyl]-3-methyl-indazole-6-carboxylic acid (Intermediate 114, 1.00 g, 2.31 mmol) in dimethylformamide (10 mL) was added HATU (1.36 g, 3.468 mmol) and DIPEA (1.21 mL, 6.936 mmol). The reaction mixture was stirred at room temperature for 15 min and then ammonium chloride (0.62 g, 11.56 mmol) was added, and the reaction stirred at room temperature for 16 h. The reaction mixture was quenched with water (50 mL) and extracted with ethyl acetate (3×50 mL). The combined organic layers were separated, dried over anhydrous Na₂SO₄ and concentrated in vacuo. The crude material was purified by combi-flash column chromatography, by eluting with 10% ethyl acetate in heptane, to afford tert-butyl 4-(2-(2-(6-carbamoyl-3-methyl-1H-indazol-1-yl)ethoxy)ethyl)piperazine-1-carboxylate (Intermediate 115, 0.80 g, 80%) as a yellow solid. ¹H NMR (400 MHz, DMSO-d₆) δ ppm 1.37 (s, 9H), 2.02-2.12 (m, 4H), 2.33 (t, J=5.37 Hz, 2H), 2.87 (s, 3H), 3.07-3.11 (m, 4H), 3.43 (t, J=5.48 Hz, 2H), 3.77 (t, J=5.26 Hz, 2H), 4.48 (t, J=5.26 Hz, 2H), 7.58 (d, J=8.44 Hz, 1H), 7.70 (d, J=8.33 Hz, 1H), 7.93 (s, 1H), 7.98 (br s, 1H), 8.13 (s, 1H).

### Step 5

### Intermediate 116: tert-butyl (R)-6-(1-(2-(2-(4-(tert-butoxycarbonyl) piperazin-1-yl)ethoxy)ethyl)-3-methyl-1H-indazole-6-carboxamido)-2-(1-methylpyrrolidin-2-yl)-1H-pyrrolo[3,2-c]pyridine-1-carboxylate

To a stirred solution of tert-butyl 6-bromo-2-[(2R)-1-methylpyrrolidin-2-yl]pyrrolo[3,2-c]pyridine-1-carboxylate (Intermediate 1, 0.40 g, 1.05 mmol) in toluene (10 mL) was added tert-butyl 4-[2-[2-(6-carbamoyl-3-methyl-indazol-1-yl)ethoxy]ethyl]piperazine-1-carboxylate (Intermediate 115, 0.36 g, 0.84 mmol) followed by cesium carbonate (1.02 g, 3.15 mmol). The reaction mixture was purged with argon for 15 min, then BrettPhos (0.05 g, 0.10 mmol) followed by BrettPhos Pd G3 (0.09 g, 0.10 mmol) were added and the reaction mixture was purged with argon for 10 min then heated at 130° C. for 3 h. The reaction mixture was diluted with water (50 mL) and extracted with EtOAc (3×60 mL). The combined organic layers were separated, dried over anhydrous Na₂SO₄ and concentrated in vacuo. The crude material was purified by combi-flash column chromatography, by eluting with 80% EtOAc in heptane, to afford tert-butyl (R)-6-(1-(2-(2-(4-(tert-butoxycarbonyl)piperazin-1-yl)ethoxy)ethyl)-3-methyl-1H-indazole-6-carboxamido)-2-(1-methylpyrrolidin-2-yl)-1H-pyrrolo[3,2-c]pyridine-1-carboxylate (Intermediate 116, 0.30 g, 39%) as an off white solid. Mass spec: m/z 731.3 [M+H+2]⁺.

### Step 6

### Intermediate 117: (R)-3-methyl-N-(2-(1-methylpyrrolidin-2-yl)-1H-pyrrolo[3,2-c]pyridin-6-yl)-1-(2-(2-(piperazin-1-yl)ethoxy)ethyl)-1H-indazole-6-carboxamide dihydrochloride

To a cooled (0° C.) solution of tert-butyl 6-[[1-[2-[2-(4-tert-butoxycarbonylpiperazin-1-yl)ethoxy]ethyl]-3-methyl-indazole-6-carbonyl]amino]-2-[(2R)-1-methylpyrrolidin-2-yl]pyrrolo[3,2-c]pyridine-1-carboxylate (Intermediate 116, 0.29 g, 0.39 mmol) in 1,4-dioxane (7 mL) was added 4M HCl in 1,4-dioxane (7 mL) and the reaction mixture was stirred at 0° C. for 15 min then at room temperature for 16 h. The reaction mixture was concentrated in vacuo to afford (R)-3-methyl-N-(2-(1-methylpyrrolidin-2-yl)-1H-pyrrolo [3,2-c]pyridin-6-yl)-1-(2-(2-(piperazin-1-yl) ethoxy) ethyl)-1H-indazole-6-carboxamide dihydrochloride (Intermediate 117, 0.25 g crude) as a yellow solid which was used for the next step without further purification. ¹H NMR (400 MHz, DMSO-d₆) δ ppm 2.12-2.28 (m, 2H), 2.33-2.44 (m, 2H), 2.53 (s, 3H), 2.54-2.57 (m, 1H), 2.82 (s, 3H), 3.17-3.19 (m, 1H), 3.22-3.34 (m, 5H), 3.35-3.45 (m, 5H), 3.82-3.84 (m, 2H), 3.94 (t, J=5.15 Hz, 2H), 4.63-4.79 (m, 3H), 7.29 (s, 1H), 7.82-7.87 (m, 1H), 7.89-7.94 (m, 1H), 8.42 (s, 1H), 8.89 (s, 1H), 9.13 (s, 1H), 9.63 (br s, 2H), 11.34 (br s, 1H), 12.27 (br s, 1H), 13.25 (br s, 1H). Mass spec: m/z 531.0 [M+H]⁺.

### Step 7

### Example 24: 1-(2-(2-(4-(2-(2,6-dioxopiperidin-3-yl)-6-fluoro-1,3-dioxoisoindolin-5-yl)piperazin-1-yl)ethoxy)ethyl)-3-methyl-N-(2-((R)-1-methylpyrrolidin-2-yl)-1H-pyrrolo[3,2-c]pyridin-6-yl)-1H-indazole-6-carboxamide

To a solution of 3-methyl-N-[2-[(2R)-1-methylpyrrolidin-2-yl]-1H-pyrrolo[3,2-c]pyridin-6-yl]-1-[2-(2-piperazin-1-ylethoxy)ethyl]indazole-6-carboxamide dihydrochloride (Intermediate 117, 0.25 g, 0.47 mmol) and 2-(2,6-dioxo-3-piperidyl)-5,6-difluoro-isoindoline-1,3-dione (CAS No: 1496997-41-1, 0.14 g, 0.47 mmol) in dimethyl sulfoxide (4 mL) was added DIPEA (0.41 mL, 2.35 mmol) and the reaction mixture was heated at 110° C. for 8 h. The reaction mixture was quenched with ice cold water (15 mL), the resultant yellow precipitate was collected by filtration and dried in vacuo. The crude material was purified by preparative HPLC (Method A) afford 1-(2-(2-(4-(2-(2,6-dioxopiperidin-3-yl)-6-fluoro-1,3-dioxoisoindolin-5-yl)piperazin-1-yl)ethoxy)ethyl)-3-methyl-N-(2-((R)-1-methylpyrrolidin-2-yl)-1H-pyrrolo[3,2-c]pyridin-6-yl)-1H-indazole-6-carboxamide (Example 24, 0.06 g, 16%) as a yellow solid. ¹H NMR (400 MHz, DMSO-d₆) δ ppm 1.80-1.88 (m, 3H), 2.02-2.05 (m, 1H), 2.12-2.14 (m, 1H), 2.15 (s, 3H), 2.24-2.27 (m, 1H), 2.38-2.43 (m, 6H), 2.52 (s, 3H), 2.55-2.62 (m, 1H), 2.85-2.89 (m, 1H), 2.98-3.05 (m, 4H), 3.11-3.17 (m, 1H), 3.25-3.29 (m, 2H), 3.52 (t, J=5.32 Hz, 2H), 3.84 (t, J=5.19 Hz, 2H), 4.60 (t, J=5.25 Hz, 2H), 5.08-5.13 (m, 1H), 6.36-6.37 (m, 1H), 7.29 (d, J=7.25 Hz, 1H), 7.59-7.60 (m, 1H), 7.71-7.80 (m, 2H), 8.23 (s, 1H), 8.48-8.49 (m, 2H), 10.47 (s, 1H), 11.10 (br s, 1H), 11.32 (br s, 1H). Mass spec: m/z 805.3 [M+H]⁺.

### Example 25 was Synthesised Following the Scheme 24

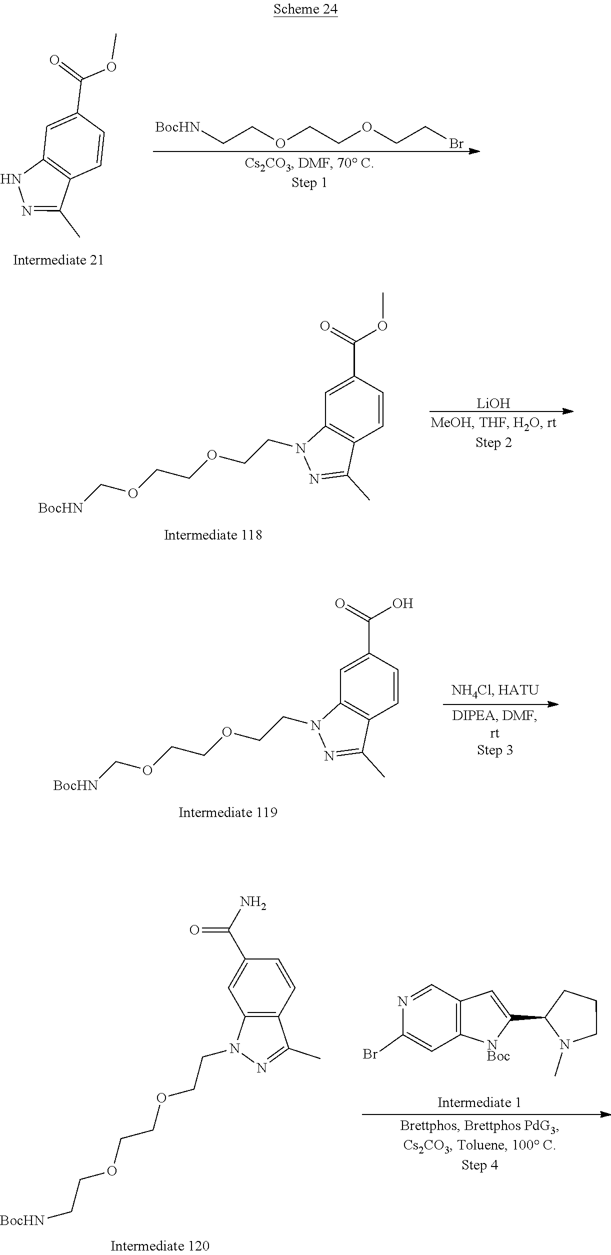

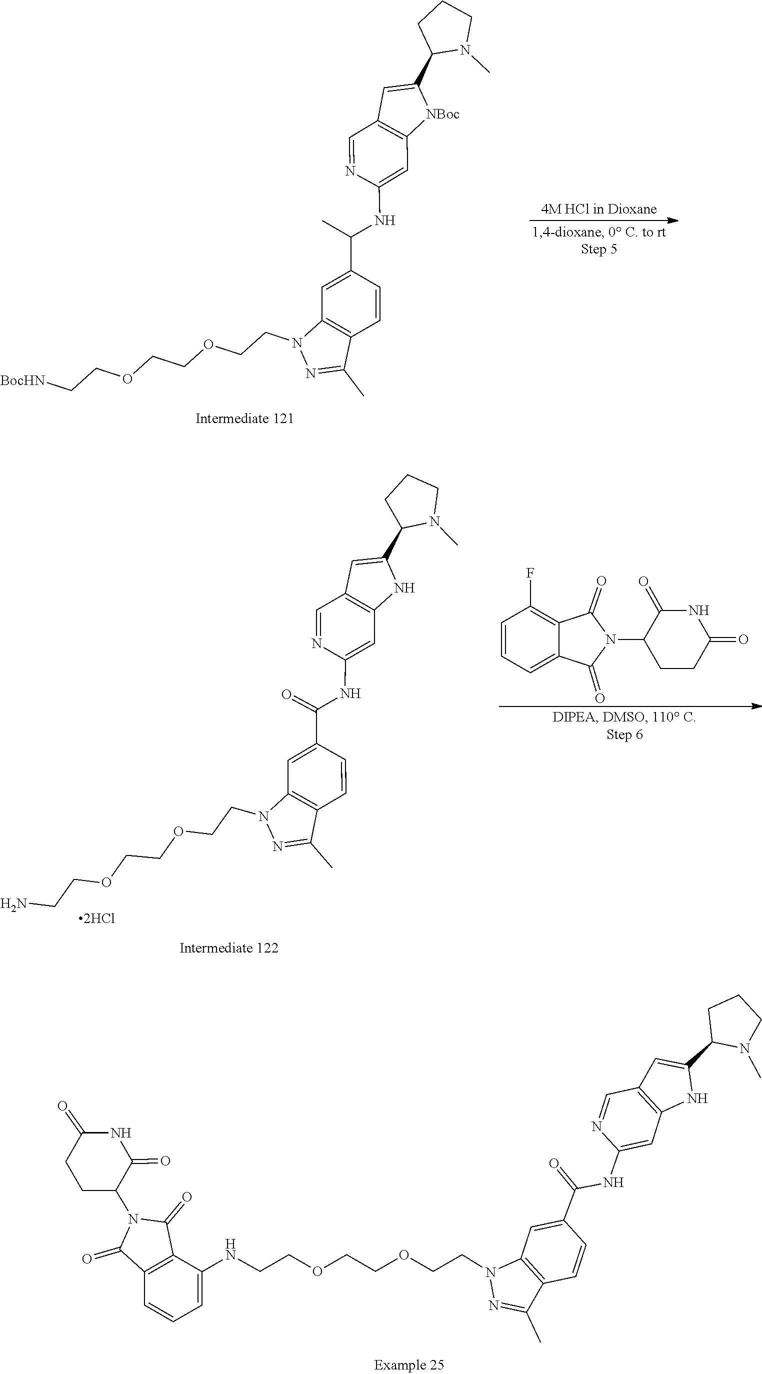 [SMILES: Cc1nn(CCOCCOCCNC(=O)OC(C)(C)C)c2cc(C(C)Nc3cc4c(cn3)cc([C@H]3CCCN3C)n4C(=O)OC(C)(C)C)ccc12 ; Cc1nn(CCOCCOCCN)c2cc(C(=O)Nc3cc4[nH]c([C@H]5CCCN5C)cc4cn3)ccc12 ; O=C1CCC(N2C(=O)c3cccc(F)c3C2=O)C(=O)N1 ; Cc1nn(CCOCCOCCNc2cccc3c2C(=O)N(C2CCC(=O)NC2=O)C3=O)c2cc(C(=O)Nc3cc4[nH]c([C@H]5CCCN5C)cc4cn3)ccc12]

### Step 1

### Intermediate 118: Methyl 1-(2,2-dimethyl-4-oxo-3,8,11-trioxa-5-azatridecan-13-yl)-3-methyl-1H-indazole-6-carboxylate

To a solution of methyl 3-methyl-1H-indazole-6-carboxylate (Intermediate 21, 1 g, 5.26 mmol) in DMF (10 mL) was added cesium carbonate (4.29 g, 13.14 mmol) followed by tert-butyl (2-(2-(2-bromoethoxy)ethoxy)ethyl)carbamate (CAS No: 1076199-21-7, 2.51 g, 7.89 mmol) and the reaction mixture was stirred at 70° C. for 16 h. The mixture was then cooled to room temperature, treated with water (50 mL) and extracted with ethyl acetate (3×50 mL). The combined organic layers were washed with brine solution (2×50 mL), dried over Na₂SO₄ and concentrated in vacuo. The resulting residue was purified by combi-flash column chromatography, by eluting with 45% ethyl acetate in heptane, to afford methyl 1-(2,2-dimethyl-4-oxo-3,8,11-trioxa-5-azatridecan-13-yl)-3-methyl-1H-indazole-6-carboxylate (Intermediate 118, 1.2 g, 54%) as a colourless liquid. ¹H NMR (400 MHz, DMSO-d₆) δ ppm 1.35 (s, 9H), 2.51 (s, 3H), 2.96 (q, J=5.46 Hz, 2H), 3.25 (t, J=6.18 Hz, 2H), 3.35-3.40 (m, 2H), 3.42-3.46 (m, 2H), 3.80 (t, J=5.24 Hz, 2H), 3.90 (s, 3H), 4.56 (t, J=5.24 Hz, 2H), 6.66 (s, 1H), 7.65 (d, J=8.33 Hz, 1H), 7.80 (d, J=8.60 Hz, 1H), 8.26 (s, 1H). Mass spec: m/z 421.9 [M+H]⁺.

### Step 2

### Intermediate 119: 1-(2,2-dimethyl-4-oxo-3,8,11-trioxa-5-azatridecan-13-yl)-3-methyl-1H-indazole-6-carboxylic acid

To a solution of methyl 1-(2,2-dimethyl-4-oxo-3,8,11-trioxa-5-azatridecan-13-yl)-3-methyl-1H-indazole-6-carboxylate (Intermediate 118, 1.2 g, 2.85 mmol) in THF (4 mL) and MeOH (4 mL) was added lithium hydroxide (0.174 g, 7.12 mmol) in water (4 mL) and the reaction mixture was stirred at room temperature for 16h. Additional water (10 mL) was then added, and the pH of the solution was adjusted to 3-4 with 0.5 N HCl and extracted with EtOAc (2×50 mL). The combined organic layers were dried over Na₂SO₄ and concentrated in vacuo. The resultant solid was washed with n-pentanes (2×30 mL) to afford 1-(2,2-dimethyl-4-oxo-3,8,11-trioxa-5-azatridecan-13-yl)-3-methyl-1H-indazole-6-carboxylic acid (Intermediate 119, 0.95 g, 82%) as an off-white solid which was used for the next step without further purification. ¹H NMR (400 MHz, DMSO-d₆) δ ppm 1.35 (s, 9H), 2.97 (s, 3H), 3.26-3.30 (m, 4H), 3.38 (d, J=4.62 Hz, 2H), 3.42-3.47 (m, 2H), 3.80 (t, J=5.09 Hz, 2H), 4.55 (t, J=5.09 Hz, 2H), 6.66 (s, 1H), 7.64 (d, J=8.32 Hz, 1H), 7.77 (d, J=8.32 Hz, 1H), 8.22 (s, 1H), 12.97 (s, 1H). Mass spec: m/z 407.9 [M+H]⁺.

### Step 3

### Intermediate 120: tert-butyl (2-(2-(2-(6-carbamoyl-3-methyl-1H-indazol-1-yl)ethoxy)ethoxy)ethyl)carbamate

To a solution of 1-(2,2-dimethyl-4-oxo-3,8,11-trioxa-5-azatridecan-13-yl)-3-methyl-1H-indazole-6-carboxylic acid (Intermediate 119, 0.93 g, 2.28 mmol) in dimethylformamide (10 mL) was added HATU (1.79 g, 4.56 mmol) and the reaction mixture was stirred at room temperature for 15 min. Ammonium chloride (0.856 g, 15.98 mmol) followed by DIPEA (1.043 g, 7.988 mmol) were then added and the reaction mixture stirred at room temperature for 16h. The reaction mixture was then treated with water (100 mL) and extracted with ethyl acetate (3×50 mL). The combined organic layers were washed sequentially with saturated aqueous NaHCO₃ solution (2×40 mL), brine solution (2×30 mL), dried over anhydrous Na₂SO₄ and concentrated in vacuo. The resultant crude solid was washed with DCM (10 mL) and n-pentane (10 mL), then dried in vacuo to afford tert-butyl (2-(2-(2-(6-carbamoyl-3-methyl-1H-indazol-1-yl)ethoxy)ethoxy)ethyl)carbamate (Intermediate 120, 0.82 g, 88%) as an off-white solid which was used for the next step without further purification. ¹H NMR (400 MHz, DMSO-d₆) 1.35 (s, 9H), 2.98 (d, J=6.01 Hz, 2H), 3.28 (t, J=6.01 Hz, 2H), 3.30 (s, 3H) 3.40 (d, J=5.09 Hz, 2H), 3.44-3.49 (m, 2H), 3.83 (t, J=5.55 Hz, 2H), 4.50 (t, J=5.55 Hz, 2H), 6.68 (s, 1H), 7.40 (s, 1H), 7.60 (d, J=8.32 Hz, 1H), 7.72 (d, J=8.32 Hz, 1H), 8.00 (s, 1H), 8.15 (s, 1H). Mass spec: m/z 406.9 [M+H]⁺.

### Step 4

### Intermediate 121: tert-butyl (R)-6-(1-(2,2-dimethyl-4-oxo-3,8,11-trioxa-5-azatridecan-13-yl)-3-methyl-1H-indazole-6-carboxamido)-2-(1-methylpyrrolidin-2-yl)-1H-pyrrolo[3,2-c]pyridine-1-carboxylate

To a solution of tert-butyl (2-(2-(2-(6-carbamoyl-3-methyl-1H-indazol-1-yl)ethoxy)ethoxy)ethyl)carbamate (Intermediate 120, 0.32 g, 0.79 mmol) in toluene (10 mL) was added tert-butyl (R)-6-bromo-2-(1-methylpyrrolidin-2-yl)-1H-pyrrolo[3,2-c]pyridine-1-carboxylate (Intermediate 1, 0.15 g, 0.39 mmol) followed by cesium carbonate (0.39 g, 1.18 mmol). The reaction mixture was purged with argon for 10 min then BrettPhos (0.044 g, 0.079 mmol) and BrettPhos Pd G3 (0.073 g, 0.079 mmol) were added. The mixture was further purged with argon gas 10 min and then heated to 100° C. for 2 h. The reaction mixture was then cooled to room temperature, diluted with water (50 mL) and extracted with ethyl acetate (3×50 mL). The combined organic layers were dried over Na₂SO₄ and concentrated in vacuo. The resulting residue was purified by combi-flash column chromatography, by eluting with 1.5% MeOH in DCM) to afford tert-butyl (R)-6-(1-(2,2-dimethyl-4-oxo-3,8,11-trioxa-5-azatridecan-13-yl)-3-methyl-1H-indazole-6-carboxamido)-2-(1-methylpyrrolidin-2-yl)-1H-pyrrolo[3,2-c]pyridine-1-carboxylate (Intermediate 121, 0.30 g, 54%) as an off-white solid. ¹H NMR (400 MHz, DMSO-d₆) δ ppm 1.30-1.34 (m, 2H), 1.37 (s, 9H), 1.69-1.70 (m, 2H), 1.71 (s, 9H), 2.31-2.40 (m, 2H), 2.52 (s, 3H), 2.93-3.01 (m, 2H), 3.27 (s, 3H), 3.37-3.43 (m, 2H), 3.44-3.52 (m, 2H), 3.80-3.97 (m, 3H), 4.50 (t, J=5.59 Hz, 1H), 4.57 (t, J=5.48 Hz, 1H), 6.76 (s, 1H), 7.77-7.81 (m, 1H), 8.01 (s, 1H), 8.15 (s, 1H), 8.46 (s, 1H), 8.61 (s, 1H), 8.98 (s, 1H), 10.73 (s, 1H). Mass spec: m/z 706 [M+H]⁺.

### Step 5

### Intermediate 122: (R)-1-(2-(2-(2-aminoethoxy)ethoxy)ethyl)-3-methyl-N-(2-(1-methylpyrrolidin-2-yl)-1H-pyrrolo[3,2-c]pyridin-6-yl)-1H-indazole-6-carboxamide dihydrochloride

To a cooled (0° C.) solution of tert-butyl (R)-6-(1-(2,2-dimethyl-4-oxo-3,8,11-trioxa-5-azatridecan-13-yl)-3-methyl-1H-indazole-6-carboxamido)-2-(1-methylpyrrolidin-2-yl)-1H-pyrrolo[3,2-c]pyridine-1-carboxylate (Intermediate 121, 0.3 g, 0.43 mmol) in 1,4-dioxane (3 mL) was added 4M HCl in 1,4-dioxane (5 mL) and the reaction mixture was allowed to warm slowly to room temperature and then stirred for 16 h. The reaction mixture then was concentrated in vacuo, washed with DCM (2×20 mL). The resultant solid was washed with diethyl ether (2×20 mL) then dried in vacuo to afford (R)-1-(2-(2-(2-aminoethoxy)ethoxy)ethyl)-3-methyl-N-(2-(1-methylpyrrolidin-2-yl)-1H-pyrrolo[3,2-c]pyridin-6-yl)-1H-indazole-6-carboxamide dihydrochloride (Intermediate 122, 0.24 g) as a white solid, which was used for next step without further purification. ¹H NMR (400 MHz, DMSO-d₆) δ ppm 2.19-2.20 (m, 2H), 2.42 (d, J=9.65 Hz, 2H), 2.55 (s, 3H), 2.80-2.89 (m, 5H), 3.48 (s, 3H), 3.70-3.73 (m, 4H), 3.90-3.91 (m, 4H), 4.60-4.63 (m, 2H), 4.76 (d, J=6.80 Hz, 1H), 7.30 (s, 1H), 7.82-8.03 (m, 4H), 8.44 (s, 1H), 8.80 (s, 1H), 9.13 (s, 1H), 11.55 (s, 1H), 12.33 (s, 1H).

### Step 6

### Example 25: 1-(2-(2-(2-((2-(2,6-dioxopiperidin-3-yl)-1,3-dioxoisoindolin-4-yl)amino)ethoxy)ethoxy)ethyl)-3-methyl-N-(2-((R)-1-methylpyrrolidin-2-yl)-1H-pyrrolo[3,2-c]pyridin-6-yl)-1H-indazole-6-carboxamide

To a solution of (R)-1-(2-(2-(2-aminoethoxy)ethoxy)ethyl)-3-methyl-N-(2-(1-methylpyrrolidin-2-yl)-1H-pyrrolo[3,2-c]pyridin-6-yl)-1H-indazole-6-carboxamide dihydrochloride (Intermediate 122, 0.28 g, 0.55 mmol) in DMSO (5 mL) was added 2-(2,6-dioxopiperidin-3-yl)-4-fluoroisoindoline-1,3-dione (CAS No: 835616-60-9, 0.15 g, 0.55 mmol) followed by DIPEA (0.29 mL, 1.66 mmol) and the reaction mixture was stirred at 110° C. for 6 h. The reaction mixture was then cooled to room temperature and diluted with water (50 mL). The resultant precipitate was collected by filtration, washed with DCM/pentane (1:1, 2×20 mL) and then dried in vacuo. The crude compound was purified by preparative HPLC (Method A) to afford 1-(2-(2-(2-((2-(2,6-dioxopiperidin-3-yl)-1,3-dioxoisoindolin-4-yl)amino)ethoxy)ethoxy)ethyl)-3-methyl-N-(2-((R)-1-methylpyrrolidin-2-yl)-1H-pyrrolo[3,2-c]pyridin-6-yl)-1H-indazole-6-carboxamide (Example 25, 60 mg, 14%) as yellow solid. ¹H NMR (400 MHz, DMSO-d₆) δ ppm 1.75-2.04 (m, 4H), 2.17 (s, 3H), 2.22-2.35 (m, 1H), 2.42-2.48 (m, 1H), 2.52 (s, 3H), 2.83-2.86 (m, 2H), 3.15 (t, J=8.07 Hz, 1H), 3.27-3.31 (m, 4H), 3.45-3.54 (m, 6H), 3.86 (t, J=5.50 Hz, 2H), 4.56 (t, J=5.44 Hz, 2H), 5.03 (dd, J=12.88, 5.38 Hz, 1H), 6.40 (s, 1H), 6.51 (t, J=6.13 Hz, 1H), 6.96-6.98 (m, 2H), 7.47 (dd, J=8.38, 7.25 Hz, 1H), 7.67-7.81 (m, 2H), 8.25 (s, 1H), 8.43-8.54 (m, 2H), 10.49 (s, 1H), 11.09 (s, 1H), 11.39 (s, 1H). Mass spec: m/z 762.2 [M+H]⁺.

### Example 26 was Synthesised Following the Scheme 25

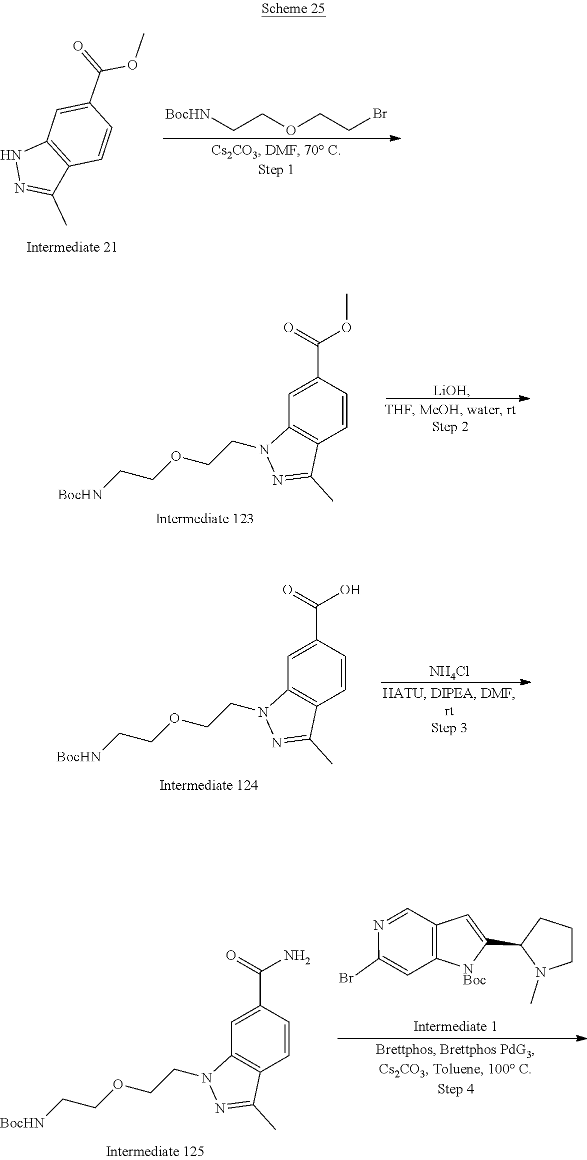

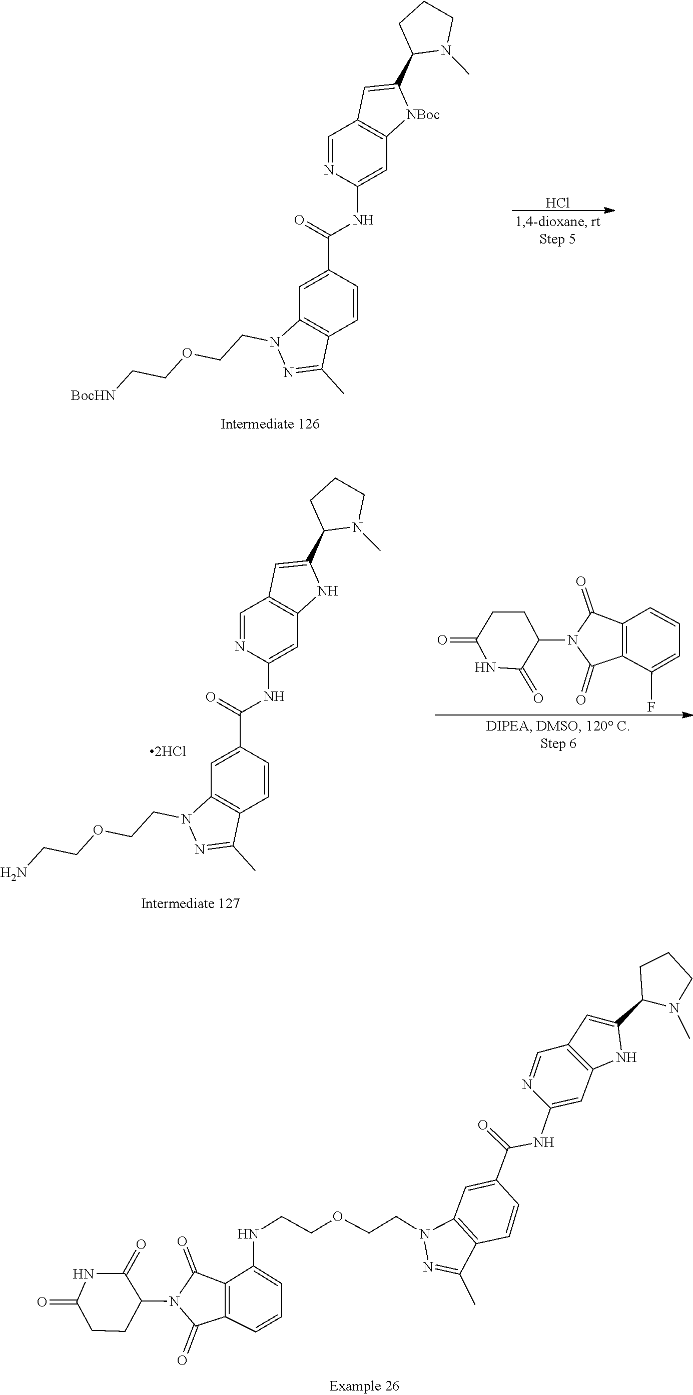 [SMILES: Cc1nn(CCOCCNC(=O)OC(C)(C)C)c2cc(C(=O)Nc3cc4c(cn3)cc([C@H]3CCCN3C)n4C(=O)OC(C)(C)C)ccc12 ; Cl ; Cc1nn(CCOCCN)c2cc(C(=O)Nc3cc4[nH]c([C@H]5CCCN5C)cc4cn3)ccc12 ; O=C1CCC(N2C(=O)c3cccc(F)c3C2=O)C(=O)N1 ; Cc1nn(CCOCCNc2cccc3c2C(=O)N(C2CCC(=O)NC2=O)C3=O)c2cc(C(=O)Nc3cc4[nH]c([C@H]5CCCN5C)cc4cn3)ccc12]

### Step 1

### Intermediate 123: Methyl 1-(2-(2-((tert-butoxycarbonyl)amino)ethoxy)ethyl)-3-methyl-1H-indazole-6-carboxylate

To a solution of methyl 3-methyl-1H-indazole-6-carboxylate (Intermediate 21, 1.5 g, 7.89 mmol) in DMF (15 mL) was added tert-butyl (2-(2-bromoethoxy)ethyl)carbamate (CAS No: 164332-88-1, 3.17 g, 11.8 mmol) followed by cesium carbonate (6.43 g, 19.7 mmol) and the reaction mixture was heated to 70° C. for 16 h. The reaction mixture was diluted with water (100 mL) and extracted with ethyl acetate (2×100 mL). The combined organic layers were separated, dried over Na₂SO₄ and concentrated in vacuo. The crude material was purified by combi-flash column chromatography, by eluting with 35% ethyl acetate in heptane, to afford methyl 1-(2-(2-((tert-butoxycarbonyl)amino)ethoxy)ethyl)-3-methyl-1H-indazole-6-carboxylate (Intermediate 123, 2.0 g, 67%) as a yellow sticky liquid. ¹H NMR (400 MHz, DMSO-d₆) δ ppm 1.34 (s, 9H), 2.51 (s, 3H), 2.97-2.99 (m, 2H), 3.27-3.35 (m, 2H), 3.78 (t, J=4.86 Hz, 2H), 3.90 (s, 3H), 4.56 (t, J=5.09 Hz, 2H), 6.54 (br s, 1H), 7.65 (d, J=8.32 Hz, 1H), 7.80 (d, J=8.32 Hz, 1H), 8.26 (s, 1H). Mass spec: m/z 378.2 [M+H]⁺.

### Step 2

### Intermediate 124: 1-(2-(2-((tert-butoxycarbonyl)amino)ethoxy)ethyl)-3-methyl-1H-indazole-6-carboxylic acid

To a solution of methyl 1-(2-(2-((tert-butoxycarbonyl)amino)ethoxy)ethyl)-3-methyl-1H-indazole-6-carboxylate (Intermediate 123, 0.65 g, 1.722 mmol) in tetrahydrofuran (2.5 mL) and methanol (2.5 mL) was added lithium hydroxide (0.25 g, 10 mmol) in water (2.5 mL). The reaction mixture was stirred at room temperature for 16 h then concentrated in vacuo to one-third volume. The pH of the aqueous layer was adjusted to 3-4 with 0.5 N HCl and extracted with EtOAc (2×50 mL). The combined organic layers were dried over Na₂SO₄ and concentrated in vacuo to afford 1-(2-(2-((tert-butoxycarbonyl)amino)ethoxy)ethyl)-3-methyl-1H-indazole-6-carboxylic acid (Intermediate 124, 0.53 g, 85%) as an off-white solid which was used for the next step without further purification. ¹H NMR (400 MHz, DMSO-d₆) δ ppm 1.34 (s, 9H), 2.52 (s, 3H), 2.94-2.99 (m, 2H), 3.30-3.34 (m, 2H), 3.78 (t, J=5.32 Hz, 2H), 4.54 (t, J=5.32 Hz, 2H), 6.55 (br s, 1H), 7.64-7.66 (m, 1H), 7.77-7.78 (m, 1H), 8.22 (s, 1H), 12.98 (br s, 1H). Mass spec: m/z 364.3 [M+H]⁺.

### Step 3

### Intermediate 125: tert-Butyl (2-(2-(6-carbamoyl-3-methyl-1H-indazol-1-yl)ethoxy)ethyl)carbamate

To a solution of 1-(2-(2-((tert-butoxycarbonyl)amino)ethoxy)ethyl)-3-methyl-1H-indazole-6-carboxylic acid (Intermediate 124, 0.70 g, 1.926 mmol) in dimethylformamide (8 mL) was added ammonium chloride (0.72 g, 13.48 mmol) and HATU (1.88 g, 4.81 mmol) followed by DIPEA (1.27 g, 9.63 mmol) and reaction mixture was stirred at room temperature for 16 h. The reaction mixture was diluted with water (100 mL) and extracted with EtOAc (2×70 mL). The combined organic layers were dried over Na₂SO₄ and concentrated in vacuo to afford tert-butyl (2-(2-(6-carbamoyl-3-methyl-1H-indazol-1-yl) ethoxy) ethyl) carbamate (Intermediate 125, 0.60 g, 86%) as an off white solid which was used for the next step without further purification. ¹H NMR (400 MHz, DMSO-d₆) δ ppm 1.34 (s, 9H), 2.50 (s, 3H), 2.92-3.03 (m, 2H), 3.30-3.37 (m, 2H), 3.76-3.82 (m, 2H), 4.49 (t, J=5.22 Hz, 2H), 6.64 (br s, 1H), 7.60 (d, J=8.31 Hz, 1H), 7.73 (d, J=8.31 Hz, 1H), 8.01 (br s, 1H), 8.15 (s, 1H). Mass spec: m/z 362.8 [M+H]⁺.

### Step 4

### Intermediate 126: tert-Butyl (R)-6-(1-(2-(2-((tert-butoxycarbonyl)amino)ethoxy)ethyl)-3-methyl-1H-indazole-6-carboxamido)-2-(1-methylpyrrolidin-2-yl)-1H-pyrrolo [3,2-c]pyridine-1-carboxylate

To a solution of tert-butyl (2-(2-(6-carbamoyl-3-methyl-1H-indazol-1-yl) ethoxy) ethyl) carbamate (Intermediate 125, 0.25 g, 0.68 mmol) in toluene (5 mL) was added tert-butyl (R)-6-bromo-2-(1-methylpyrrolidin-2-yl)-1H-pyrrolo[3,2-c]pyridine-1-carboxylate (Intermediate 1, 0.20 g, 0.55 mmol) and cesium carbonate (0.67 g, 2.070 mmol). The reaction mixture was purged with argon gas for 10 min then BrettPhos (0.057 mg, 0.10 mmol) and BrettPhos Pd G3 (0.095 g, 0.10 mmol) were added and the mixture was purged again with argon gas for 10 min, then heated to 100° C. for 1 h. The reaction mixture was cooled to room temperature, treated with water (50 mL), and extracted with ethyl acetate (3×50 mL). The combined organic layers were dried over Na₂SO₄ and concentrated in vacuo. The crude material was purified by combi-flash column chromatography, by eluting with 80% ethyl acetate in heptane, to afford tert-butyl (R)-6-(1-(2-(2-((tert-butoxycarbonyl)amino)ethoxy)ethyl)-3-methyl-1H-indazole-6-carboxamido)-2-(1-methylpyrrolidin-2-yl)-1H-pyrrolo[3,2-c]pyridine-1-carboxylate (Intermediate 126, 0.20 g, 44%) as a yellow solid. Mass spec: m/z 661.9 [M+H]⁺.

### Step 5

### Intermediate 127: (R)-1-(2-(2-aminoethoxy) ethyl)-3-methyl-N-(2-(1-methylpyrrolidin-2-yl)-1H-pyrrolo [3,2-c]pyridin-6-yl)-1H-indazole-6-carboxamide dihydrochloride

To a cooled (0° C.) solution of tert-butyl (R)-6-(1-(2-(2-((tert-butoxycarbonyl)amino)ethoxy)ethyl)-3-methyl-1H-indazole-6-carboxamido)-2-(1-methylpyrrolidin-2-yl)-1H-pyrrolo[3,2-c]pyridine-1-carboxylate (Intermediate 126, 0.20 g, 0.30 mmol) in 1,4-dioxane (5 mL) was added 4 M HCl in 1,4dioxane (5 mL). The reaction mixture was allowed to stir to room temperature for 16 h. The reaction mixture was concentrated in vacuo, the resultant precipitate was collected by filtration, washed with diethyl ether (2×20 mL) and dried in vacuo to afford crude (R)-1-(2-(2-aminoethoxy) ethyl)-3-methyl-N-(2-(1-methylpyrrolidin-2-yl)-1H-pyrrolo [3,2-c]pyridin-6-yl)-1H-indazole-6-carboxamide dihydrochloride (Intermediate 127, 0.16 g) as a yellow solid which was directly used in the next step without purification. ¹H NMR (400 MHz, DMSO-d₆) δ ppm 1.23-1.25 (m, 2H), 1.58-1.61 (m, 1H), 2.20-2.22 (m, 2H), 2.56 (s, 3H), 2.82 (s, 3H), 2.90-2.92 (m, 3H), 3.70-3.85 (m, 4H), 3.94-3.96 (m, 3H), 4.66-4.68 (br s, 3H), 7.24 (br s, 1H), 7.84 (br s, 1H), 7.89 (br s, 1H), 8.42 (s, 1H), 8.84 (s, 1H), 9.08 (s, 1H), 11.27 (br s, 1H), 12.2 (s, 1H) 13.09 (br s, 1H). Mass spec: m/z 461.8 [M+H]⁺.

### Step 6

### Example 26: 1-(2-(2-((2-(2,6-dioxopiperidin-3-yl)-1,3-dioxoisoindolin-4yl)amino)ethoxy)ethyl)-3-methyl-N-(2-((R)-1-methylpyrrolidin-2-yl)-1H-pyrrolo[3,2-c]pyridin-6-yl)-1H-indazole-6-carboxamide

To a solution of (R)-1-(2-(2-aminoethoxy) ethyl)-3-methyl-N-(2-(1-methylpyrrolidin-2-yl)-1H-pyrrolo [3,2-c]pyridin-6-yl)-1H-indazole-6-carboxamide dihydrochloride (Intermediate 127, 0.157 g, 0.34 mmol) in DMSO (5.00 mL) was added 2-(2,6-dioxopiperidin-3-yl)-4-fluoroisoindoline-1,3-dione (CAS No: 835616-60-9, 0.093 g, 0.3401 mmol) and DIPEA (0.135 g, 1.020 mmol). The reaction mixture was heated at 110° C. for 5 h. The reaction mixture was cooled to room temperature, treated with cold water (50 mL) and the resultant yellow precipitate was collected by filtration and dried in vacuo. The crude compound was purified by preparative HPLC (Method A) to afford 1-(2-(2-((2-(2,6-dioxopiperidin-3-yl)-1,3-dioxoisoindolin-4-yl)amino)ethoxy)ethyl)-3-methyl-N-(2-((R)-1-methylpyrrolidin-2-yl)-1H-pyrrolo[3,2-c]pyridin-6-yl)-1H-indazole-6-carboxamide (Example 26, 0.035 g, 14%) as a yellow solid. ¹H NMR (400 MHz, DMSO-d₆) δ ppm 1.77-1.94 (m, 3H), 1.97-2.03 (m, 1H), 2.15-2.16 (m, 1H), 2.17 (s, 3H), 2.27-2.28 (m, 1H), 2.38-2.48 (m, 1H), 2.57 (s, 3H), 2.80-2.90 (m, 1H), 3.08-3.21 (m, 1H), 3.28-3.31 (m, 1H), 3.38-3.40 (m, 3H), 3.60 (t, J=5.44 Hz, 2H), 3.93 (t, J=5.57 Hz, 2H), 4.58 (t, J=5.69 Hz, 2H), 5.01-5.03 (m, 1H), 6.40 (s, 1H), 6.50 (t, J=5.88 Hz, 1H), 6.97-6.99 (m, 2H), 7.41-7.48 (m, 1H), 7.70-7.77 (m, 2H), 8.24 (s, 1H), 8.47 (s, 1H), 8.51 (s, 1H), 10.48 (s, 1H), 11.09 (s, 1H), 11.39 (s, 1H). Mass spec: m/z 717.2 [M+H]⁺.

### Example 27 was Made Following Scheme 26

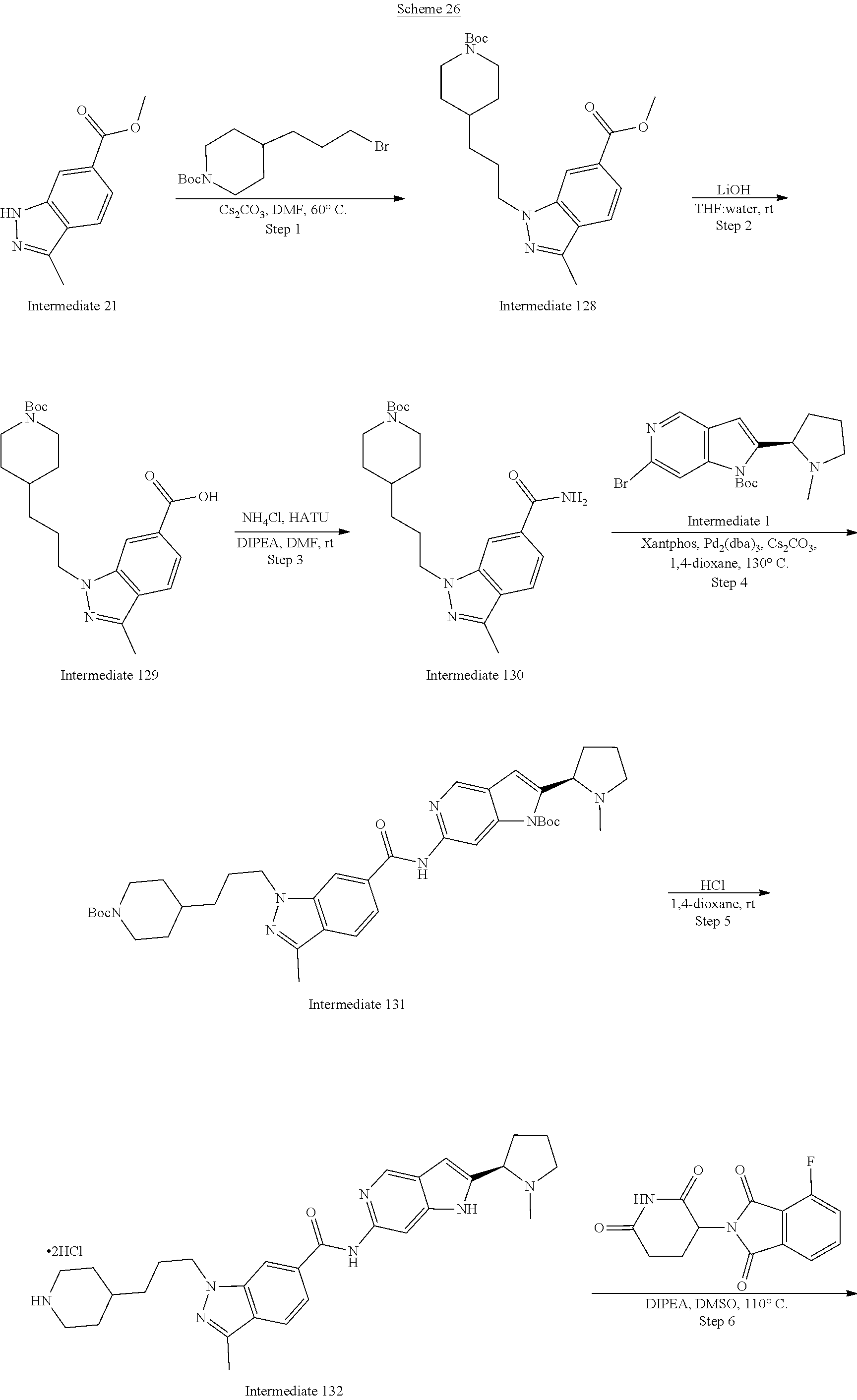

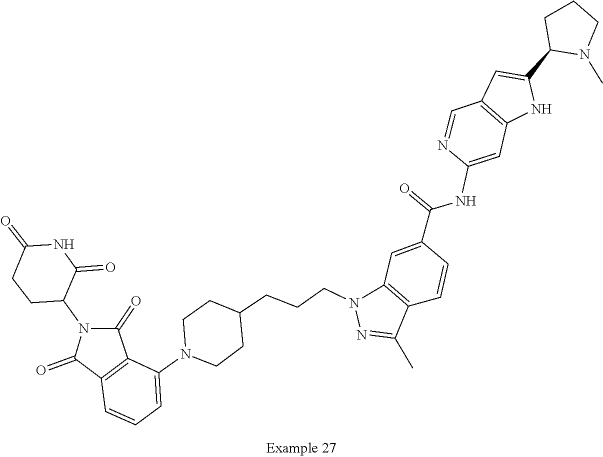 [SMILES: Cc1nn(CCCC2CCN(c3cccc4c3C(=O)N(C3CCC(=O)NC3=O)C4=O)CC2)c2cc(C(=O)Nc3cc4[nH]c([C@H]5CCCN5C)cc4cn3)ccc12]

### Step 1

### Intermediate 128: methyl 1-(3-(1-(tert-butoxycarbonyl)piperidin-4-yl) propyl)-3-methyl-1H-indazole-6-carboxylate

To a cooled (0C) solution of methyl 3-methyl-H-indazole-6-carboxylate (Intermediate 21, 5.00 g, 26.28 mmol) in DMF (50 mL) was added cesium carbonate (17.1 g, 52.57 mmol) followed by tert-butyl 4-(3-bromopropyl)piperidine-carboxylate (CAS No: 164149-27-3, 8.04 g, 26.28 mmol) and the reaction mixture was heated to 80° C. for 16 h. The reaction mixture was then cooled to room temperature, treated with water (60 mL) and extracted with ethyl acetate (2×100 mL). The combined organic layers were dried over anhydrous Na₂SO₄ and concentrated in vacuo. The crude material was purified by combi-flash column chromatography, by eluting with 15% ethyl acetate in heptane, to afford methyl 1-(3-(1-(tert-butoxycarbonyl) piperidin-4-yl)propyl)-3-methyl-1H-indazole-6-carboxylate (Intermediate 128, 6.0 g, 55%) as an off white solid. ¹H NMR (400 MHz, DMSO-d₆) δ ppm 0.86-0.97 (m, 3H), 1.11-1.23 (m, 2H), 1.37 (s, 9H), 1.54-1.57 (m, 2H), 1.77-1.83 (m, 2H), 2.51 (s, 3H), 2.63-2.65 (m, 2H), 3.82-3.87 (m, 2H), 3.90 (s, 3H), 4.39 (t, J=7.02 Hz, 2H), 7.65 (d, J=8.33, 1H), 7.81 (d, J=8.33 Hz, 1H), 8.23 (s, 1H). Mass spec: m/z 416.3 [M+H]⁺.

### Step 2

### Intermediate 129: 1-(3-(1-(tert-butoxycarbonyl) piperidin-4-yl) propyl)-3-methyl-1H-indazole-6-carboxylic acid

To a solution of methyl 1-(3-(1-(tert-butoxycarbonyl) piperidin-4-yl)propyl)-3-methyl-1H-indazole-6-carboxylate (Intermediate 128, 6.00 g, 14.44 mmol) in THF (30 mL) and methanol (30 mL) was added lithium hydroxide (3.03 g, 72.20 mmol) in water (15 mL). The reaction mixture was stirred at room temperature for 4 h and then concentrated in vacuo. Water (30 mL) was added, the mixture acidified to pH~3 using aqueous saturated citric acid solution and the resultant precipitate was collected by filtration and dried in vacuo to afford to afford tert-butyl 4-(3-(6-carbamoyl-3-methyl-1H-indazol-1-yl) propyl) piperidine-1-carboxylate (Intermediate 129, 5.0 g, 86%) as an off-white solid which was used for the next step without further purification. ¹H NMR (400 MHz, DMSO-d₆) δ ppm 0.84-0.93 (m, 2H), 1.14-1.20 (m, 3H), 1.37 (s, 9H), 1.52-1.58 (m, 2H), 1.78-1.85 (m, 2H), 2.50 (s, 3H), 2.62-2.73 (m, 2H), 3.86-3.89 (m, 2H), 4.37 (t, J=6.91 Hz, 2H), 7.64 (d, J=8.33 Hz, 1H), 7.78 (d, J=8.33 Hz, 1H), 8.19 (s, 1H), 13.02 (br s, 1H). Mass spec: m/z 402.3 [M+H]⁺.

### Step 3

### Intermediate 130: tert-butyl 4-(3-(6-carbamoyl-3-methyl-1H-indazol-1-yl) propyl)piperidine-1-carboxylate

To a solution of 1-(3-(1-(tert-butoxycarbonyl)piperidin-4-yl)propyl)-3-methyl-1H-indazole-6-carboxylic acid (Intermediate 129, 5.00 g, 12.45 mmol) in dimethylformamide (50 mL) was added HATU (5.85 g, 14.94 mmol) and DIPEA (6.53 mL, 37.36 mmol). The reaction mixture was stirred at room temperature for 15 min. Ammonium chloride (0.7 g, 62.27 mmol) was then added and the reaction mixture was stirred at room temperature for 16 h. The reaction mixture was then treated with water (200 mL) and extracted with EtOAc (3×150 mL). The combined organic layers were washed with NaHCO₃ (2×60 mL) and brine solution (2×60 mL), dried over Na₂SO₄ and concentrated in vacuo. The crude material was purified by combi-flash column chromatography, by eluting with 70% ethyl acetate in heptane, to afford tert-butyl 4-(3-(6-carbamoyl-3-methyl-1H-indazol-1-yl)propyl) piperidine-1-carboxylate (Intermediate 130, 2.7 g, 54%) as an off-white solid. Mass spec: m/z 401.3 [M+H]⁺.

### Step 4

### Intermediate 131: tert-butyl-(R)-6-(1-(3-(1-(tert-butoxycarbonyl)piperidin-4-yl)propyl)-3-methyl-1H-indazole-6-carboxamido)-2-(1-methylpyrrolidin-2-yl)-1H-pyrrolo [3,2-c]pyridine-1-carboxylate

To a solution of tert-butyl 4-(3-(6-carbamoyl-3-methyl-1H-indazol-1-yl)propyl)piperidine-1-carboxylate (Intermediate 130, 0.42 g, 1.05 mmol) in 1,4-dioxane (10 mL) was added tert-butyl-(R)-6-bromo-2-(1-methylpyrrolidin-2-yl)-1H-pyrrolo[3,2-c]pyridine-1-carboxylate (Intermediate 1, 0.5 g, 1.31 mmol) followed by cesium carbonate (1.28 g, 3.94 mmol). The reaction mixture was purged with argon for 10 min then Pd₂(dba)₃ (0.18 g, 0.19 mmol) and 4,5-bis(diphenylphosphino)-9,9-dimethylxanthene (0.22 g, 0.39 mmol) was added and the reaction mixture was heated at 130° C. for 2 h. The reaction mixture was then diluted with water (100 mL) and extracted with ethyl acetate (2×100 mL). The combined organic layers were separated, dried over anhydrous Na₂SO₄ and concentrated in vacuo. The crude material was purified by combi-flash chromatography, by eluting with 70% ethyl acetate in heptane, to afford tert-butyl-(R)-6-(1-(3-(1-(tert-butoxycarbonyl)piperidin-4-yl)propyl)-3-methyl-1H-indazole-6-carboxamido)-2-(1-methylpyrrolidin-2-yl)-1H-pyrrolo[3,2-c]pyridine-1-carboxylate (Intermediate 131, 0.3 g, 33%) as a yellow solid. Mass spec: m/z 700 [M+H]⁺.

### Step 5

### Intermediate 132: (R)-3-methyl-N-(2-(1-methylpyrrolidin-2-yl)-1H-pyrrolo[3,2-c]pyridin-6-yl)-1-(3-(piperidin-4-yl)propyl)-1H-indazole-6-carboxamide dihydrochloride

To a 0° C. solution of (R)-6-(1-(3-(1-(tert-butoxycarbonyl)piperidin-4-yl)propyl)-3-methyl-1H-indazole-6-carboxamido)-2-(1-methylpyrrolidin-2-yl)-1H-pyrrolo[3,2-c]pyridine-1-carboxylate (Intermediate 131, 0.6 g, 0.85 mmol) in 1,4-dioxane (8 mL) was added 4M HCl in 1,4-dioxane (8 mL). The reaction mixture was stirred at room temperature for 16 h and then concentrated in vacuo to afford (R)-3-methyl-N-(2-(1-methylpyrrolidin-2-yl)-1H-pyrrolo[3,2-c]pyridin-6-yl)-1-(3-(piperidin-4-yl)propyl)-1H-indazole-6-carboxamide dihydrochloride (Intermediate 132, 0.46 g) as a yellow solid, which was used in next step without further purification. ¹H NMR (400 MHz, DMSO-d₆) δ ppm 1.29-1.35 (m, 4H), 1.58-1.60 (m, 1H), 1.74-1.77 (m, 2H), 1.82-1.98 (m, 2H), 2.18 (s, 3H), 2.19-2.22 (m, 2H), 2.55 (s, 3H), 2.74-2.87 (m, 4H), 3.15-3.31 (m, 4H), 4.46 (t, J=6.51 Hz, 2H), 4.72-4.75 (m, 1H), 7.27 (s, 1H), 7.81-7.83 (m, 1H), 7.79-7.91 (m, 1H), 8.43 (br s, 2H), 8.80 (br s, 2H), 9.10 (s, 1H), 11.32 (br s, 1H), 12.25 (br s, 1H), 13.17 (br s, 1H). Mass spec: m/z 500.1 [M+H]⁺.

### Step 6

### Example 27: 1-(3-(1-(2-(2,6-dioxopiperidin-3-yl)-1,3-dioxoisoindolin-4-yl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide

To a solution of (R)-3-methyl-N-(2-(1-methylpyrrolidin-2-yl)-1H-pyrrolo [3,2-c]pyridin-6-yl)-1-(3-(piperidin-4-yl)propyl)-1H-indazole-6-carboxamide dihydrochloride (Intermediate 132, 0.30 g, 0.60 mmol) in dimethyl sulfoxide (4 mL) was added 2-(2,6-dioxopiperidin-3-yl)-4-fluoroisoindoline-1,3-dione (CAS No: 835616-60-9, 0.16 g, 0.60 mmol) followed by DIPEA (0.29 mL, 3.00 mmol) and the reaction mixture was stirred at 110° C. for 8 h. The reaction mixture was then quenched with ice cold water, a yellow precipitate was obtained which was filtered and dried in vacuo. The crude material was then purified by preparative HPLC (Method A) to afford 1-(3-(1-(2-(2,6-dioxopiperidin-3-yl)-1,3-dioxoisoindolin-4-yl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide (Example 27, 0.07 g, 15%) as a yellow solid. ¹H NMR (400 MHz, DMSO-d₆) δ ppm 1.29-1.35 (m, 4H), 1.46-1.48 (m, 1H), 1.72-1.85 (m, 3H), 1.89-2.02 (m, 5H), 2.18 (s, 3H), 2.24-2.30 (m, 1H), 2.53 (s, 3H), 2.55-2.59 (m, 2H), 2.80-2.86 (m, 3H), 3.13-3.17 (m, 1H), 3.32-3.34 (m, 2H), 3.65-3.68 (m, 2H), 4.43 (t, J=6.88 Hz, 2H), 5.05-5.09 (m, 1H), 6.40 (s, 1H), 7.28-7.30 (m, 2H), 7.62-7.66 (m, 1H), 7.72-7.76 (m, 1H), 7.80 (d, J=8.50 Hz, 1H), 8.25 (s, 1H), 8.45 (s, 1H), 8.52 (s, 1H), 10.53 (s, 1H), 11.39 (s, 1H). Mass spec: m/z 756.2 [M+H]⁺.

### Example 28 was Made Following Scheme 27

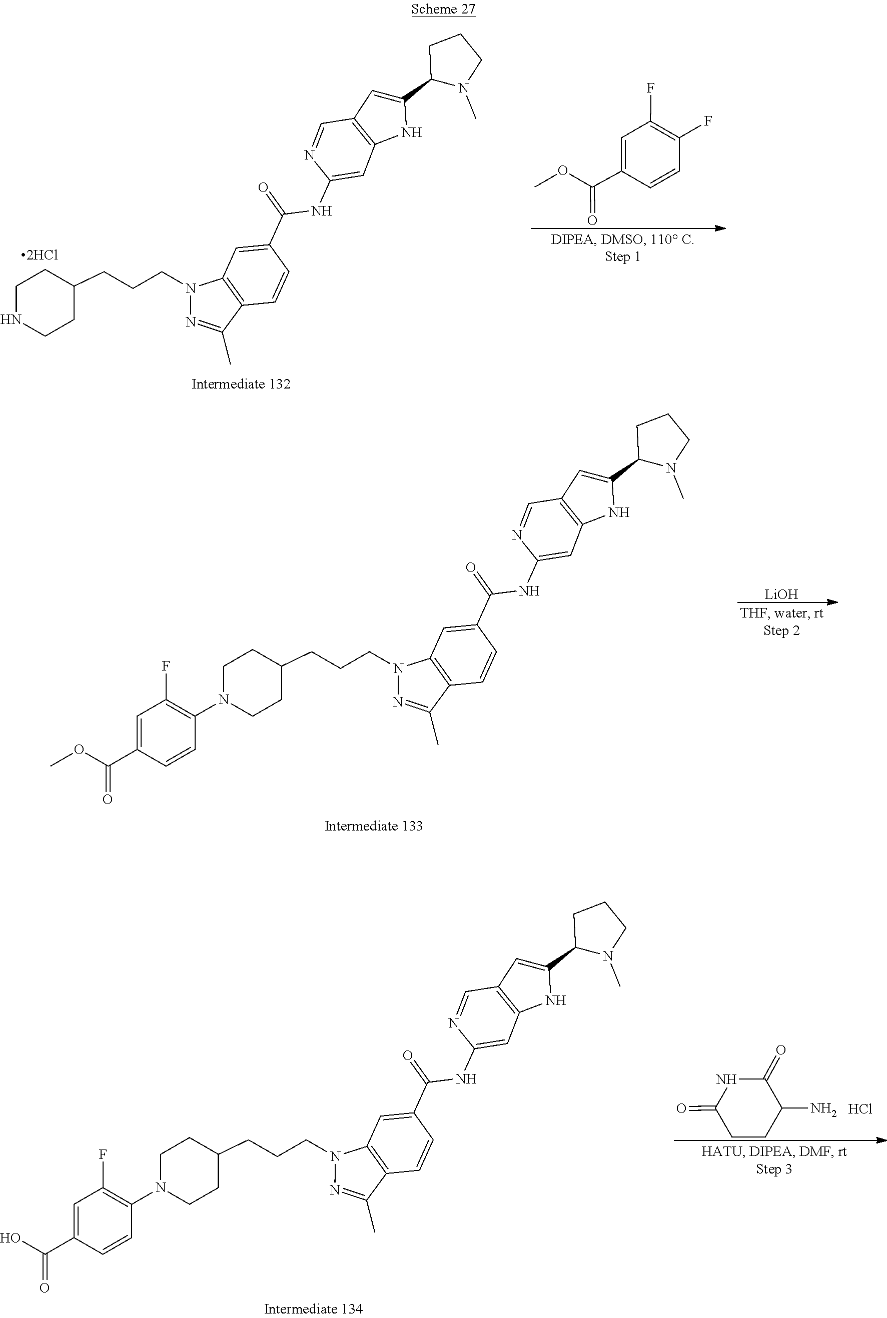

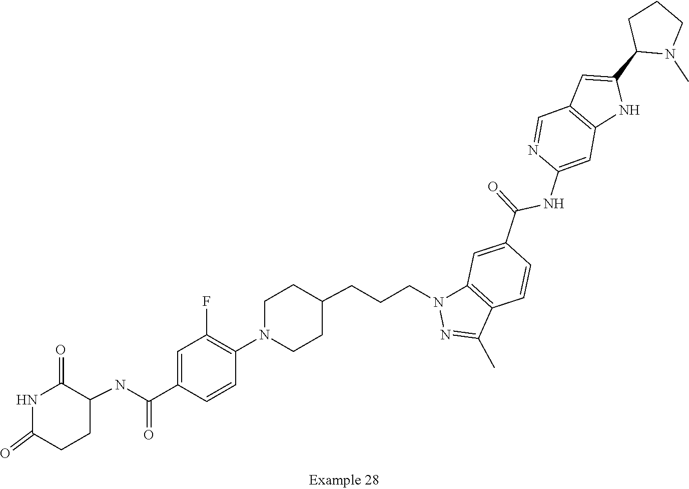

### Step 1

### Intermediate 133: methyl (R)-3-fluoro-4-(4-(3-(3-methyl-6-((2-(1-methylpyrrolidin-2-yl)-1H-pyrrolo[3,2-c]pyridin-6-yl)carbamoyl)-1H-indazol-1-yl)propyl)piperidin-1-yl)benzoate

To a suspension of (R)-3-methyl-N-(2-(1-methylpyrrolidin-2-yl)-1H-pyrrolo [3,2-c]pyridin-6-yl)-1-(3-(piperidin-4-yl)propyl)-1H-indazole-6-carboxamide dihydrochloride (Intermediate 132, 300 mg, 0.60 mmol) and methyl 3,4-difluorobenzoate (CAS No: 369-25-5, 0.124 g) in DMSO (3.0 mL) was added DIPEA (1.80 g, 1.801 mmol) and the reaction mixture was heated at 80° C. for 16h. Ice cold water (5 mL) was added and the resultant precipitate was collected by filtration and dried in vacuo to afford methyl (R)-3-fluoro-4-(4-(3-(3-methyl-6-((2-(1-methylpyrrolidin-2-yl)-1H-pyrrolo[3,2-c]pyridin-6-yl)carbamoyl)-1H-indazol-1-yl)propyl)piperidin-1-yl)benzoate (Intermediate 133, 0.17 g, 43%) as a pale yellow solid which was used for the next step without further purification. ¹H NMR (400 MHz, DMSO-d₆) δ ppm 1.17-1.32 (m, 4H), 1.69-1.98 (m, 4H), 2.13-2.19 (m, 4H), 2.22-2.33 (m, 1H), 2.54 (s, 3H), 2.68-2.78 (m, 2H) 3.15-3.17 (m, 1H) 3.37 (s, 3H), 3.49-3.50 (m, 2H), 3.79 (s, 3H), 4.11-4.13 (m, 2H), 4.41-4.42 (m, 2H), 6.41 (s, 1H), 7.04 (t, J=8.82 Hz, 1H), 7.53-7.57 (m, 2H), 7.72-7.75 (m, 1H), 7.77-7.81 (m, 1H), 8.24 (s, 1H), 8.43 (s, 1H), 8.52 (s, 1H) 10.51 (s, 1H), 11.38 (br s, 1H). Mass spec: m/z 652.3 [M+H]⁺.

### Step 2

### Intermediate 134: (R)-3-fluoro-4-(4-(3-(3-methyl-6-((2-(1-methylpyrrolidin-2-yl)-1H-pyrrolo[3,2-c]pyridin-6-yl)carbamoyl)-1H-indazol-1-yl)propyl)piperidin-1-yl)benzoic acid

A solution of (R)-3-fluoro-4-(4-(3-(3-methyl-6-((2-(1-methylpyrrolidin-2-yl)-1H-pyrrolo[3,2-c]pyridin-6-yl)carbamoyl)-1H-indazol-1-yl)propyl)piperidin-1-yl)benzoate (Intermediate 133, 0.17 g, 0.26 mmol) and LiOH (0.021 g, 0.52 mmol) in THF (3 mL), methanol (2 mL) and water (2 mL) was stirred at room temperature for 4 h. The reaction mixture was concentrated in vacuo to one-third volume and water (10 mL) was added. The pH was adjusted to pH~2 by addition of aqueous citric acid solution and the resultant white precipitate was filtered and dried in vacuo to afford (R)-3-fluoro-4-(4-(3-(3-methyl-6-((2-(1-methylpyrrolidin-2-yl)-1H-pyrrolo[3,2-c]pyridin-6-yl)carbamoyl)-1H-indazol-1-yl)propyl)piperidin-1-yl)benzoic acid (Intermediate 134, 0.13 g, 78%) as off white solid which was used for the next step without further purification. ¹H NMR (400 MHz, DMSO-d₆) δ ppm 1.15-1.32 (m, 7H), 1.35-1.47 (m, 2H), 2.16-2.18 (m, 3H), 2.19 (s, 3H), 2.22-2.31 (m, 2H), 2.54 (s, 3H), 2.60-2.72 (m, 3H), 3.11-3.17 (m, 2H), 4.00-4.06 (m, 1H), 4.42-4.44 (m, 2H), 6.39-6.47 (m, 2H), 7.43 (s, 1H), 7.72-7.81 (m, 2H), 8.25 (s, 1H), 8.43-8.55 (m, 3H), 10.54 (s, 1H), 11.40 (s, 1H), 11.60 (br s, 1H). Mass spec: m/z 638.36 [M+H]⁺.

### Step 3

### Example 28: 1-(3-(1-(4-((2,6-dioxopiperidin-3-yl)carbamoyl)-2-fluorophenyl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide

To a solution of (R)-3-fluoro-4-(4-(3-(3-methyl-6-((2-(1-methylpyrrolidin-2-yl)-1H-pyrrolo[3,2-c]pyridin-6-yl)carbamoyl)-1H-indazol-1-yl)propyl)piperidin-1-yl)benzoic acid (Intermediate 134, 0.15 g, 0.23 mmol) in DMF (3.00 mL) was added HATU (0.11 g 0.28 mmol) and DIPEA (0.093 g, 0.70 mmol) and the reaction mixture was stirred at room temperature for 15 min. 3-Aminopiperidine-2,6-dione hydrochloride (CAS No: 24666-56-6, 0.042 g, 0.25 mmol) was added and the reaction mixture was stirred at room temperature for 16 h. The reaction mixture was then diluted with water (100 mL) and extracted with EtOAc (3×50 mL). The combined organic layers were washed with NaHCO₃ (2×40 mL) and brine (2×30 mL), then dried over anhydrous Na₂SO₄ and concentrated in vacuo. The crude material was purified by preparative HPLC (Method A) to afford (R)-3-fluoro-4-(4-(3-(3-methyl-6-((2-(1-methylpyrrolidin-2-yl)-1H-pyrrolo[3,2-c]pyridin-6-yl)carbamoyl)-1H-indazol-1-yl)propyl)piperidin-1-yl)benzoic acid (Example 28, 0.015 g, 9%) as off-white solid. ¹H NMR (400 MHz, DMSO-d₆) δ ppm 1.23-1.31 (m, 4H), 1.42-1.44 (m, 1H), 1.71-1.83 (m, 3H), 1.81-1.96 (m, 5H), 2.07-2.14 (m, 2H), 2.17 (s, 3H), 2.23-2.30 (m, 1H), 2.53 (s, 3H), 2.66-2.69 (m, 2H), 2.73-2.81 (m, 1H), 3.12-3.17 (m, 1H), 3.24-3.31 (m, 1H), 3.33-3.35 (m, 1H), 3.44-3.47 (m, 2H), 4.42 (t, J=6.94 Hz, 2H), 4.71-4.78 (m, 1H), 6.41 (s, 1H), 7.05-7.10 (m, 1H), 7.57-7.63 (m, 2H), 7.74 (d, J=8.51, 1H), 7.80 (d, J=8.50 Hz, 1H), 8.25 (s, 1H), 8.45 (s, 1H), 8.52 (s, 1H), 8.63-8.64 (m, 1H), 10.54 (s, 1H), 10.84 (s, 1H), 11.40 (s, 1H). Mass spec: m/z: 748.4 [M+H]⁺.

### Example 29 was Synthesised Following Scheme 28

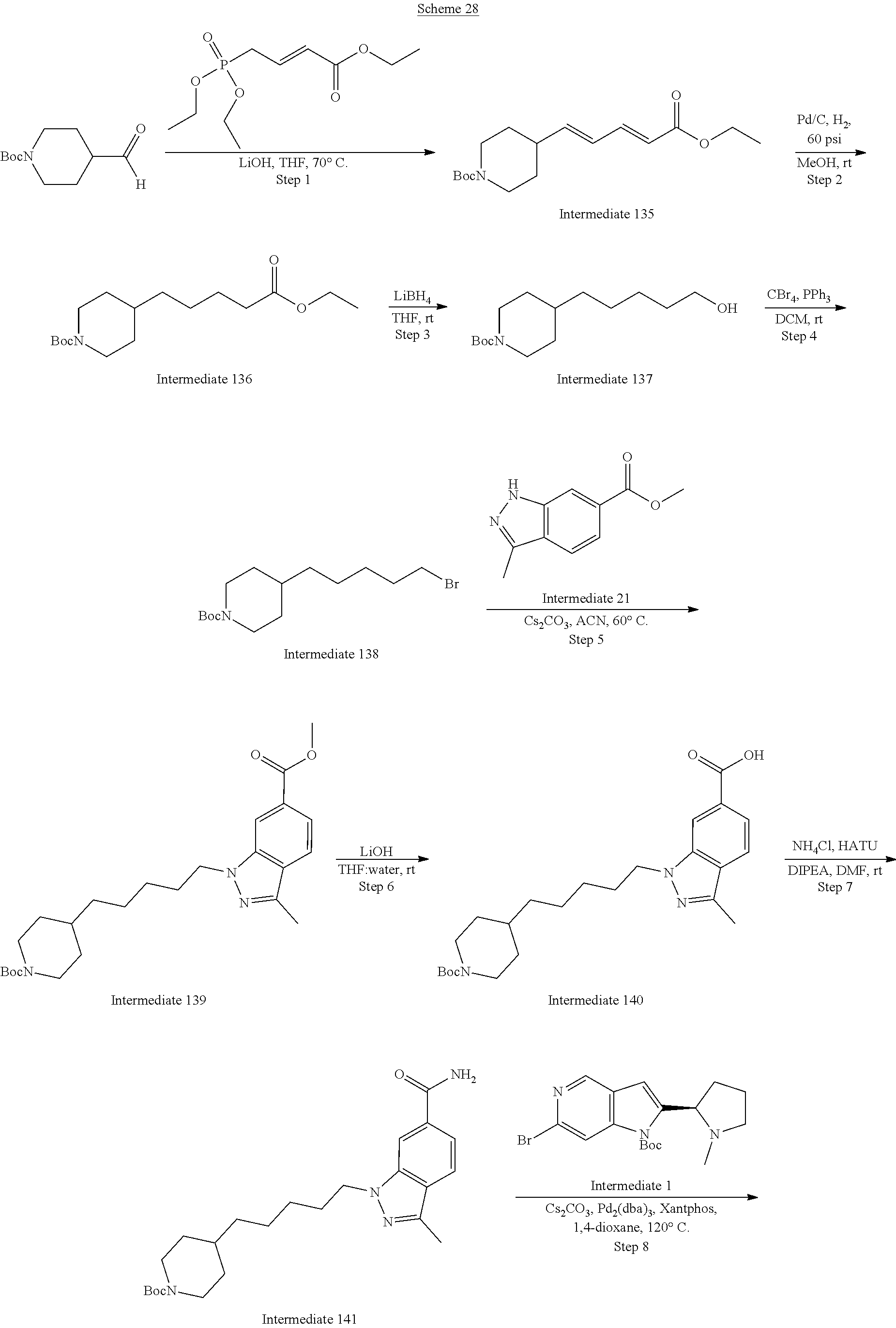

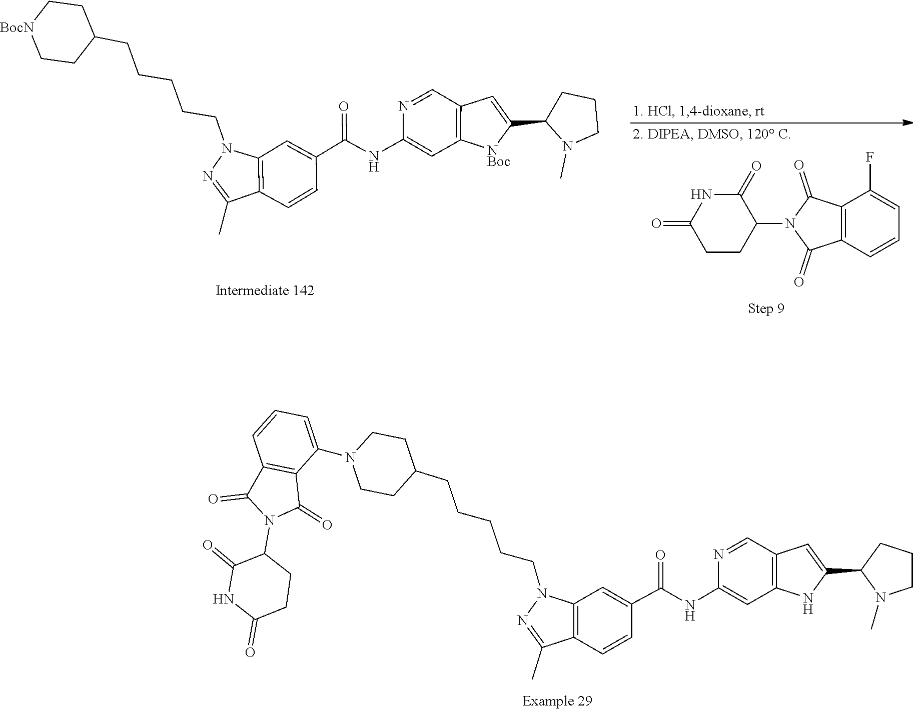 [SMILES: Cc1nn(CCCCCC2CCN(C(=O)OC(C)(C)C)CC2)c2cc(C(=O)Nc3cc4c(cn3)cc([C@H]3CCCN3C)n4C(=O)OC(C)(C)C)ccc12 ; O=C1CCC(N2C(=O)c3cccc(F)c3C2=O)C(=O)N1 ; Cc1nn(CCCCCC2CCN(c3cccc4c3C(=O)N(C3CCC(=O)NC3=O)C4=O)CC2)c2cc(C(=O)Nc3cc4[nH]c([C@H]5CCCN5C)cc4cn3)ccc12]

### Step 1

### Intermediate 135: tert-butyl 4-((1E,3E)-5-ethoxy-5-oxopenta-1,3-dien-1-yl) piperidine-1-carboxylate

To a solution of tert-butyl 4-formylpiperidine-1-carboxylate (CAS No: 137076-22-3, 10.0 g, 46.9 mmol) and ethyl (E)-4-(diethoxyphosphoryl)but-2-enoate (CAS No: 42516-28-9, 11.7 g, 46.9 mmol) in tetrahydrofuran (100 mL) was added lithium hydroxide (1.35 g, 56.3 mmol) and the reaction mixture was heated at 80° C. for 2 h. The reaction mixture was diluted with water (50 mL), resulting in a precipitate, which was filtered and dried to obtain crude compound. The crude material was purified by combi-flash column chromatography by eluting with 30% EtOAc in heptane to afford tert-butyl 4-((1E,3E)-5-ethoxy-5-oxopenta-1,3-dien-1-yl) piperidine-1-carboxylate (Intermediate 135, 10.0 g, 70%) as a yellow liquid. Mass spec: m/z: 209.9 [M+H−100]⁺.

### Step 2

### Intermediate 136: tert-butyl 4-(5-ethoxy-5-oxopentyl) piperidine-1-carboxylate

To a solution of tert-butyl 4-((1E,3E)-5-ethoxy-5-oxopenta-1,3-dien-1-yl)piperidine-1-carboxylate (Intermediate 135, 12.00 g, 40.62 mmol) in methanol (120 mL) was added 10% Pd/C (3.2 g, 30 mmol) at room temperature and the reaction mixture was stirred for 6 h under H₂ atmosphere at 60 psi. The reaction mixture was filtered through celite and concentrated in vacuo. The crude material was purified by combi-flash column chromatography by eluting with 30% EtOAc in heptane to afford tert-butyl 4-(5-ethoxy-5-oxopentyl) piperidine-1-carboxylate (Intermediate 136, 9.6 g, 79%) as a colorless liquid. ¹H NMR (400 MHz, DMSO-d₆) δ ppm 0.87-0.90 (m, 2H), 1.15-1.20 (m, 6H), 1.23-1.31 (m, 1H), 1.38 (s, 9H), 1.46-1.53 (m, 2H), 1.58-1.61 (m, 2H), 2.27 (t, J=7.23 Hz, 2H), 2.62-2.65 (m, 2H), 3.89-3.91 (m, 2H), 4.01-4.06 (m, 2H). Mass spec: m/z: 214.5 [M+H-100].

### Step 3

### Intermediate 137: tert-butyl 4-(5-hydroxypentyl)piperidine-1-carboxylate

To a solution of tert-butyl 4-(5-methoxy-5-oxopentyl) piperidine-1-carboxylate (Intermediate 136, 3.80 g, 13 mmol) in tetrahydrofuran (70 mL) was added lithium borohydride (2.50 g, 100 mmol) slowly at 0° C. and the reaction stirred for 30 min. The reaction mixture was then stirred at room temperature for 2 h. The reaction mixture was quenched with cold water (50 mL) and extracted with ethyl acetate (2×100 mL). The combined organic phases were washed with water (200 mL) and brine solution (100 mL), dried over Na₂SO₄ and concentrated in vacuo. The obtained crude was purified by combi-flash column chromatography by eluting with 75% ethyl acetate in heptane to afford tert-butyl 4-(5-hydroxypentyl) piperidine-1-carboxylate (Intermediate 137, 3.1 g, 90%) as a liquid. ¹H NMR (400 MHz, DMSO-d₆) δ ppm 0.86-0.99 (m, 4H), 1.15-1.19 (m, 1H), 1.38 (s, 9H), 1.58-1.62 (m, 4H), 1.90-1.98 (m, 1H), 2.24-2.28 (m, 1H), 3.37 (t, J=6.36 Hz, 2H), 3.66-3.70 (m, 2H), 3.88-3.92 (m, 4H), 4.01-4.06 (m, 1H). Mass spec: m/z: 272.2 [M+H]⁺.

### Step 4

### Intermediate 138: tert-butyl 4-(5-bromopentyl)piperidine-1-carboxylate

To a solution of tert-butyl 4-(5-hydroxypentyl)piperidine-1-carboxylate (Intermediate 137, 2.50 g, 9.2 mmol) in dichloromethane (25 mL) was added triphenylphosphine (3.0 g, 11 mmol) followed by carbon tetrabromide (5.0 g, 15 mmol) at 0° C. and the reaction mixture was stirred at room temperature for 2 h. The reaction mixture was diluted with water (50 mL) and extracted with DCM (2×100 mL). The combined organic phases were washed with water (2×60 mL) and brine solution (2×60 mL), dried over Na₂SO₄ and concentrated in vacuo. The crude material was purified by combi-flash column chromatography by eluting with 35% EtOAc in heptane to afford tert-butyl 4-(5-bromopentyl) piperidine-1-carboxylate (Intermediate 138, 1.80 g, 58%) as a liquid. Mass spec: m/z: 278.08 [M−56]⁺.

### Step 5

### Intermediate 139: methyl 1-(5-(1-(tert-butoxycarbonyl)piperidin-4-yl)pentyl)-3-methyl-1H-indazole-6-carboxylate

To a solution of methyl 3-methyl-1H-indazole-6-carboxylate (Intermediate 21, 1.0 g, 5.0 mmol) in acetonitrile (25 mL) was added cesium carbonate (4.0 g, 10.0 mmol) followed by tert-butyl 4-(5-bromopentyl)piperidine-1-carboxylate (Intermediate 138, 2.0 g, 6.0 mmol) and the reaction mixture was heated at 60° C. for 16 h. The reaction mixture was quenched with cold water (50 mL) and extracted with ethyl acetate (2×100 mL). The combined organic layers were washed with water (200 mL) and brine solution (100 mL), dried over Na₂SO₄ and concentrated in vacuo to obtain crude compound. Purification by combi-flash chromatography by eluting with 30% ethyl acetate in heptane to afforded methyl 1-(5-(1-(tert-butoxycarbonyl)piperidin-4-yl)pentyl)-3-methyl-1H-indazole-6-carboxylate (Intermediate 139, 1.6 g, 70%) as a yellow liquid. ¹H NMR (400 MHz, DMSO-d₆) δ ppm 0.83-0.93 (m, 2H), 1.08-1.17 (m, 2H), 1.17-1.27 (m, 5H), 1.37 (s, 9H), 1.53-1.56 (m, 2H), 1.76-1.83 (m, 2H), 2.51 (s, 3H), 2.56-2.62 (m, 2H), 3.83-3.86 (m, 2H), 3.90 (s, 3H), 4.40-4.42 (m, 2H), 7.65 (d, J=8.31 Hz, 1H), 7.81 (d, J=8.31 Hz, 1H), 8.22 (s, 1H). Mass spec: m/z: 444.2 [M+H]⁺.

### Step 6

### Intermediate 140: 1-(5-(1-(tert-butoxycarbonyl) piperidin-4-yl) pentyl)-3-methyl-1H-indazole-6-carboxylic acid

To a solution of methyl 1-(5-(1-(tert-butoxycarbonyl)piperidin-4-yl) pentyl)-3-methyl-1H-indazole-6-carboxylate (Intermediate 139, 1.5 g, 3.4 mmol) in tetrahydrofuran (10 mL) and methanol (5 mL) was added lithium hydroxide (0.25 g, 10.0 mmol) in water (5 mL) at 0° C. and the reaction mixture was stirred at room temperature for 24 h. The reaction mixture was concentrated in vacuo. The reaction mixture was then diluted with water (50 mL), adjusted to pH ~4 with 1N HCl, resulting in a precipitate, which was filtered and dried to afford 1-(5-(1-(tert-butoxycarbonyl)piperidin-4-yl) pentyl)-3-methyl-1H-indazole-6-carboxylic acid (Intermediate 140, 1.3 g, 89%) as a yellow liquid, which was used in the next step without further purification. ¹H NMR (400 MHz, DMSO-d₆) δ ppm 1.39 (s, 9H), 1.89-1.92 (m, 5H), 2.30-2.33 (m, 2H), 2.51 (s, 3H), 2.56-2.60 (m, 3H), 2.63-2.70 (m, 2H), 3.12-3.16 (m, 2H), 3.49-3.51 (m, 2H), 3.77-3.80 (m, 2H), 4.57 (t, J=4.80 Hz, 2H), 7.64 (d, J=8.52 Hz, 1H), 7.77 (d, J=8.40 Hz, 1H), 8.22 (s, 1H).

### Step 7

### Intermediate 141: tert-butyl 4-(5-(6-carbamoyl-3-methyl-1H-indazol-1-yl) pentyl) piperidine-1-carboxylate

To a solution of 1-(5-(1-(tert-butoxycarbonyl)piperidin-4-yl)pentyl)-3-methyl-1H-indazole-6-carboxylic acid (Intermediate 140, 1.3 g, 3.0 mmol) in dimethylformamide (10 mL) was added HATU (1.8 g, 4.5 mmol) and the reaction mixture was stirred at room temperature for 15 min. Ammonium chloride (0.81 g, 15.0 mmol) and DIPEA (2.6 mL, 15.0 mmol) were added and the reaction and stirred at room temperature for 12 h. The reaction mixture was diluted with water (50 mL) and extracted with EtOAc (3×50 mL). The combined organic phases were dried over Na₂SO₄ and concentrated in vacuo. The crude material was purified by combi-flash column chromatography by eluting with 50% EtOAc in heptane to afford tert-butyl 4-(5-(6-carbamoyl-3-methyl-1H-indazol-1-yl) pentyl) piperidine-1-carboxylate (Intermediate 141, 1.1 g, 85%) as a yellow solid. ¹H NMR (400 MHz, DMSO-d₆) δ ppm 0.84-0.92 (m, 2H), 1.11-1.13 (m, 2H), 1.12-1.26 (m, 6H), 1.37 (s, 9H), 1.53-1.57 (m, 2H), 1.79-1.82 (m, 2H), 2.50 (s, 3H), 2.60-2.68 (m, 2H), 3.85-3.89 (m, 2H), 4.31-4.35 (m, 2H), 7.42 (br s, 1H), 7.59 (d, J=8.40 Hz, 1H), 7.73 (d, J=8.40 Hz, 1H), 8.03 (br s, 1H), 8.12 (s, 1H). Mass spec: m/z: 427.2 [M−H]—.

### Step 8

### Intermediate 142: tert-butyl-(R)-6-(1-(5-(1-(tert-butoxycarbonyl)piperidin-4-yl)pentyl)-3-methyl-1H-indazole-6-carboxamido)-2-(1-methylpyrrolidin-2-yl)-1H-pyrrolo[3,2-c]pyridine-1-carboxylate

To a solution of tert-butyl 4-(5-(6-carbamoyl-3-methyl-1H-indazol-1-yl)pentyl)piperidine-1-carboxylate (Intermediate 141, 0.4 g, 0.93 mmol) n 1,4-dioxane (5 mL) was added tert-butyl (R)-6-bromo-2-(1-methylpyrrolidin-2-yl)-1H-pyrrolo[3,2-c]pyridine-1-carboxylate (Intermediate 1, 0.35 g, 0.93 mmol) followed by cesium carbonate (1.0 g, 3.08 mmol) and the reaction mixture was purged with argon for 10 min. Pd₂(dba₃ (1.32 g, 1.40 mmol) and Xantphos (0.13 g, 0.14 mmol) were added and the reaction mixture was further purged with argon for 10 min. and the reaction mixture heated at 120° C. for 16 h. The reaction mixture was diluted with water (20 mL) and extracted with EtOAc (3×25 mL). The combined organic phases were dried over Na₂SO₄ and concentrated in vacuo. The crude material was purified by combi-flash column chromatography by eluting with 80% EtOAc in heptane to afford tert-butyl (R)-6-(1-(5-(1-(tert-butoxycarbonyl)piperidin-4-yl)pentyl)-3-methyl-1H-indazole-6-carboxamido)-2-(1-methylpyrrolidin-2-yl)-1H-pyrrolo[3,2-c]pyridine-1-carboxylate (Intermediate 142, 0.4 g, 66%) as a yellow solid. Mass spec: m/z: 626.3 [M+H-100]*.

### Step 9

### Example 29: 1-(5-(1-(2-(2,6-dioxopiperidin-3-yl)-1,3-dioxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide

To a solution of tert-butyl (R)-6-(1-(5-(1-(tert-butoxycarbonyl)piperidin-4-yl)pentyl)-3-methyl-1H-indazole-6-carboxamido)-2-(1-methylpyrrolidin-2-yl)-1H-pyrrolo[3,2-c]pyridine-1-carboxylate (Intermediate 142, 0.33 g, 0.45 mmol) in 1,4-dioxane (10 mL), 4M HCl in 1,4-dioxane (5 mL) was added at 0° C. and the reaction mixture was stirred at room temperature for 4 h. The reaction mixture was concentrated in vacuo to afford as (R)-3-methyl-N-(2-(1-methylpyrrolidin-2-yl)-1H-pyrrolo[3,2-c]pyridin-6-yl)-1-(5-(piperidin-4-yl)pentyl)-1H-indazole-6-carboxamide dihydrochloride (1.35 g) as a white solid which was used for the next step without further purification. To (R)-3-methyl-N-(2-(1-methylpyrrolidin-2-yl)-1H-pyrrolo[3,2-c]pyridin-6-yl)-1-(5-(piperidin-4-yl)pentyl)-1H-indazole-6-carboxamide dihydrochloride (0.3 g, 0.56 mmol) in dimethyl sulfoxide (5 mL) was added 2-(2,6-dioxopiperidin-3-yl)-4-fluoroisoindoline-1,3-dione (CAS No: 835616-60-9, 0.16 g, 0.56 mmol) followed by N,N-DIPEA (0.22 g, 1.70 mmol) and the reaction mixture was heated at 120° C. for 3 h. The reaction mixture was diluted with water (50 mL), resulting in a precipitate, which was filtered and dried. The crude material was purified by reverse phase preparative HPLC (Method A) method to afford 1-(5-(1-(2-(2,6-dioxopiperidin-3-yl)-1,3-dioxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide (Example 29, 0.12 g, 30%) as a yellow solid. ¹H NMR (400 MHz, DMSO-d) δ ppm 1.19-1.37 (m, 9H), 1.70-1.74 (m, 2H), 1.78-1.94 (m, 5H), 2.00-2.02 (m, 1H), 2.11-2.15 (m, 1H), 2.17 (s, 3H), 2.25-2.28 (m, 1H), 2.52 (s, 3H), 2.56-2.60 (m, 2H), 2.74-2.79 (m, 2H), 2.85-2.92 (m, 1H), 3.12-3.17 (m, 1H), 3.33-3.35 (m, 1H), 3.60-3.63 (m, 2H), 4.42 (t, J=7.00 Hz, 2H), 5.04-5.09 (m, 1H), 6.40 (s, 1H), 7.22 (d, J=8.80 Hz, 1H), 7.29 (d, J=6.80 Hz, 1H), 7.59-7.63 (m, 1H), 7.72-7.76 (m, 1H), 7.77-7.81 (m, 1H), 8.26 (s, 1H), 8.46 (s, 1H), 8.52 (s, 1H), 10.54 (s, 1H), 11.06 (s, 1H), 11.38 (s, 1H). Mass spec: m/z: 784.4 [M+H]⁺.

### Example 30 was Synthesized Following Scheme 29

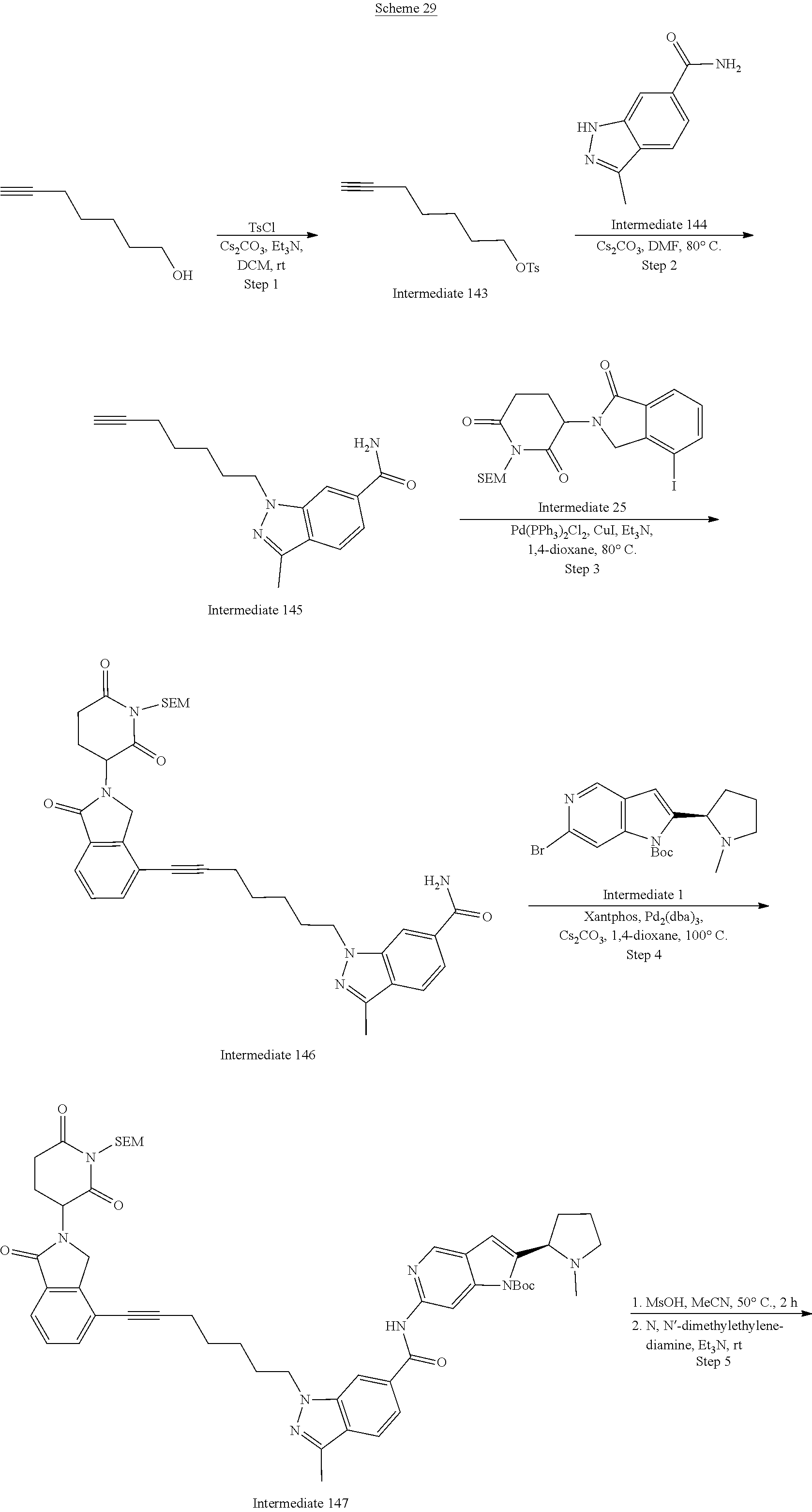

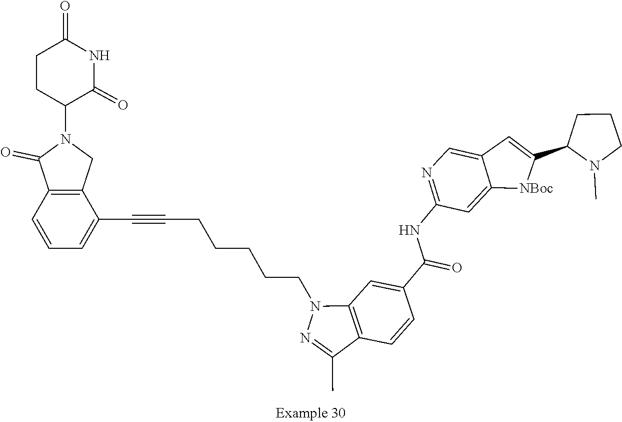 [SMILES: Cc1nn(CCCCCC#Cc2cccc3c2CN(C2CCC(=O)NC2=O)C3=O)c2cc(C(=O)Nc3cc4c(cn3)cc([C@H]3CCCN3C)n4C(=O)OC(C)(C)C)ccc12]

### Step 1

### Intermediate 143: hept-6-yn-1-yl 4-methylbenzenesulfonate

To cooled (0° C.) solution of hept-6-yn-1-ol (CAS No. 63478-76-2, 25 g, 222.88 mmol) in dichloromethane (250 mL) was added triethylamine (93.7 mL, 668.63 mmol) followed by cesium carbonate (218 g, 668.63 mmol) and p-toluenesulfonyl chloride (65.0 g, 334.31 mmol) and the reaction mixture was stirred at room temperature for 16 h. The reaction mixture was diluted with water (200 mL) and extracted with dichloromethane 2×200 mL). The combined organic layers were dried over anhydrous Na₂SO₄ and concentrated in vacuo. The crude material was purified by combi-flash chromatography, by eluting with 30% ethyl acetate in heptane, to afford hept-6-yn-1-yl 4-methylbenzenesulfonate (Intermediate 143, 35 g, 59%) as a colourless liquid. ¹H NMR (400 MHz, DMSO-d₆) δ ppm 1.29-1.33 (m, 4H), 1.51-1.59 (m, 2H), 2.06-2.09 (m, 2H), 2.42 (s, 3H), 2.74 (s, 1H), 4.00 (t, J=6.01 Hz, 2H), 7.48 (d, J=7.88 Hz, 2H), 7.78 (d, J=7.88 Hz, 2H). Mass spec: m/z 266.97 [M+H]⁺.

### Step 2

### Intermediate 145: 1-(hept-6-yn-1-yl)-3-methyl-1H-indazole-6-carboxamide

To a solution of hept-6-yn-1-yl 4-methylbenzenesulfonate (Intermediate 143, 7.00 g, 26.28 mmol) in DMF (70 mL) was added cesium carbonate (25.7 g, 78.83 mmol) followed by 3-methyl-1H-indazole-6-carboxamide (Intermediate 144, 4.60 g, 26.28 mmol) and the reaction mixture was heated at 80° C. for 16 h. The reaction mixture was diluted with water (100 mL) and extracted with ethyl acetate (3×100 mL). The combined organic layers were dried over anhydrous Na₂SO₄ and concentrated in vacuo. The crude material was purified by combi-flash chromatography, by eluting with 50% ethyl acetate in heptane, to afford 1-(hept-6-yn-1-yl)-3-methyl-1H-indazole-6-carboxamide (Intermediate 145, 2.5 g, 35%) as an off white solid. ¹H NMR (400 MHz, DMSO-d₆) δ ppm 1.31-1.35 (m, 2H), 1.45-1.49 (m, 2H), 1.80-1.84 (m, 2H), 2.11-2.17 (m, 2H), 2.51 (s, 3H), 2.71 (s, 1H), 4.34 (t, J=6.63 Hz, 2H), 7.41 (br s, 1H), 7.59 (d, J=8.71 Hz, 1H), 7.73 (d, J=8.29 Hz, 1H), 8.02 (br s, 1H), 8.12 (s, 1H). Mass spec: m/z 270.1 [M+H]⁺.

### Step 3

### Intermediate 146: 1-(7-(2-(2,6-dioxo-1-((2-(trimethylsilyl) ethoxy) methyl) piperidin-3-yl)-1-oxoisoindolin-4-yl) hept-6-yn-1-yl)-3-methyl-1H-indazole-6-carboxamide

To a solution of 1-(hept-6-yn-1-yl)-3-methyl-1H-indazole-6-carboxamide (Intermediate 145, 2.00 g, 7.43 mmol) in 1,4-dioxane (20 mL) was added copper iodide (0.14 g, 0.74 mmol) and dichlorobis(triphenylphosphine)palladium(II) (2.69 g, 3.71 mmol) followed by 3-(4-iodo-1-oxoisoindolin-2-yl)-1-((2-(trimethylsilyl)ethoxy)methyl)piperidine-2,6-dione (Intermediate 25, 3.72 g, 7.43 mmol) and triethylamine (5.20 mL, 37.13 mmol). The reaction mixture was stirred at room temperature for 2 h then diluted with water (100 mL) and extracted with ethyl acetate (3×100 mL). The combined organic layers were dried over anhydrous Na₂SO₄ and concentrated in vacuo. The crude material was purified by combi-flash chromatography, by eluting with 60% ethyl acetate in heptane, to afford 1-(7-(2-(2,6-dioxo-1-((2-(trimethylsilyl) ethoxy) methyl) piperidin-3-yl)-1-oxoisoindolin-4-yl) hept-6-yn-1-yl)-3-methyl-1H-indazole-6-carboxamide (Intermediate 146, 2.50 g, 51%) as a yellow solid. ¹H NMR (400 MHz, DMSO-d₆) δ ppm −0.05 (s, 9H), 0.79-0.83 (m, 2H), 1.15-1.19 (m, 1H), 1.41-1.45 (m, 2H), 1.55-1.63 (m, 2H), 1.83-1.89 (m, 2H), 2.03-2.06 (m, 1H), 2.41-2.44 (m, 3H), 2.46 (s, 3H), 2.72-2.78 (m, 1H), 3.02-3.09 (m, 2H), 3.47-3.53 (m, 1H), 4.22-4.27 (m, 1H), 4.34-4.45 (m, 2H), 4.98-5.06 (m, 2H), 5.23-5.27 (m, 1H), 7.41 (br s, 1H), 7.46-7.61 (m, 3H), 7.71-7.73 (m, 2H), 8.02 (br s, 1H), 8.13 (s, 1H). Mass spec: m/z 640.2 [M+H]⁺.

### Step 4

### Intermediate 147: tert-butyl-6-(1-(7-(2-(2,6-dioxo-1-((2-(trimethylsilyl)ethoxy)methyl)piperidin-3-yl)-1-oxoisoindolin-4-yl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(7-(2-(2,6-dioxo-1-((2-(trimethylsilyl)ethoxy)methyl) piperidin-3-yl)-1-oxoisoindolin-4-yl) hept-6-yn-1-yl)-3-methyl-1H-indazole-6-carboxamide (Intermediate 146, 0.80 g, 1.24 mmol) in 1,4-dioxane (10 mL) was added cesium carbonate (1.22 g, 3.74 mmol) followed by tert-butyl-(R)-6-bromo-2-(1-methylpyrrolidin-2-yl)-1H-pyrrolo[3,2-c]pyridine-1-carboxylate (Intermediate 1, 0.47 g, 1.25 mmol) and the reaction mixture was purged with argon gas for 5 minutes. Dichloro[1,3-bis(2,6-di-3-pentylphenyl)imidazol-2-ylidene](3-chloropyridyl)palladium(II) (0.13 g, 0.16 mmol) and Xantphos (0.07 g, 0.12 mmol) were then added and the reaction mixture was again purged with argon gas for 5 minutes then heated at 100° C. for 2 h. The reaction mixture was concentrated in vacuo to obtain crude compound which was purified by combi-flash chromatography, by eluting with 80% ethyl acetate in heptane, to afford tert-butyl 6-(1-(7-(2-(2,6-dioxo-1-((2-(trimethylsilyl)ethoxy)methyl)piperidin-3-yl)-1-oxoisoindolin-4-yl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 147, 0.80 g, 62%) as an off white solid. ¹H NMR (400 MHz, DMSO-d₆) δ ppm −0.06 (s, 9H), 0.78-0.84 (m, 2H), 1.45-1.48 (m, 2H), 1.62-1.66 (m, 3H), 1.70 (s, 9H), 1.73-1.84 (m, 3H), 1.94-2.05 (m, 4H), 2.36 (s, 3H), 2.47-2.49 (s, 5H), 2.72-2.77 (m, 2H), 3.39-3.51 (m, 4H), 3.91-4.04 (m, 1H), 4.21-4.26 (m, 1H), 4.39-4.43 (m, 3H), 4.96-5.05 (m, 2H), 5.23-5.26 (m, 1H), 6.76 (s, 1H), 7.47-7.51 (m, 1H), 7.53 (d, J=7.60 Hz, 1H), 7.69-7.73 (m, 2H), 7.77 (d, J=8.40 Hz, 1H), 8.45 (s, 1H), 8.60 (s, 1H), 8.97 (s, 1H), 10.73 (s, 1H).

### Step 5

### Example 30: 1-(7-(2-(2,6-dioxopiperidin-3-yl)-1-oxoisoindolin-4-yl) hept-6-yn-1-yl)-3-methyl-N-(2-((R)-1-methylpyrrolidin-2-yl)-1H-pyrrolo [3,2-c]pyridin-6-yl)-1H-indazole-6-carboxamide

To a solution of tert-butyl-6-(1-(7-(2-(2,6-dioxo-1-((2-(trimethylsilyl)ethoxy)methyl)piperidin-3-yl)-1-oxoisoindolin-4-yl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 147, 0.50 g, 0.53 mmol) in acetonitrile (15.0 mL) was added methanesulfonic acid (0.78 g, 7.96 mmol) and the reaction mixture was heated at 50° C. for 2 h. The reaction mixture was cooled to room temperature then N,N′-dimethylethylenediamine (0.31 g, 3.18 mmol) followed by triethylamine (1.08 g, 10.6 mmol) were added and the reaction mixture was stirred at room temperature for 3 h. The reaction mixture was concentrated in vacuo to obtain crude compound which was purified by preparative HPLC (Method A) to afford 1-(7-(2-(2,6-dioxopiperidin-3-yl)-1-oxoisoindolin-4-yl) hept-6-yn-1-yl)-3-methyl-N-(2-((R)-1-methylpyrrolidin-2-yl)-1H-pyrrolo [3,2-c]pyridin-6-yl)-1H-indazole-6-carboxamide (Example 30, 0.040 g, 11%) as an off white solid. ¹H NMR (400 MHz, DMSO-d₆) δ ppm 1.43-1.49 (m, 2H), 1.59-1.66 (m, 2H), 1.79-2.01 (m, 6H), 2.13-2.16 (m, 1H), 2.18 (s, 3H), 2.24-2.30 (m, 1H), 2.40-2.45 (m, 2H), 2.48 (s, 3H), 2.56-2.58 (m, 2H), 2.84-2.93 (m, 1H), 3.13-3.17 (m, 1H), 3.32-3.34 (m, 1H), 4.26-4.31 (m, 1H), 4.39-4.45 (m, 3H), 5.09-5.14 (m, 1H), 6.40 (s, 1H), 7.48-7.49 (m, 1H), 7.54-7.56 (m, 1H), 7.67-7.78 (m, 3H), 8.24 (s, 1H), 8.45 (s, 1H), 8.51 (s, 1H), 10.51 (s, 1H), 10.98 (s, 1H), 11.38 (s, 1H). Mass spec: m/z 711.2 [M+H]⁺.

### Intermediate 144 was Synthesised Following Scheme 30

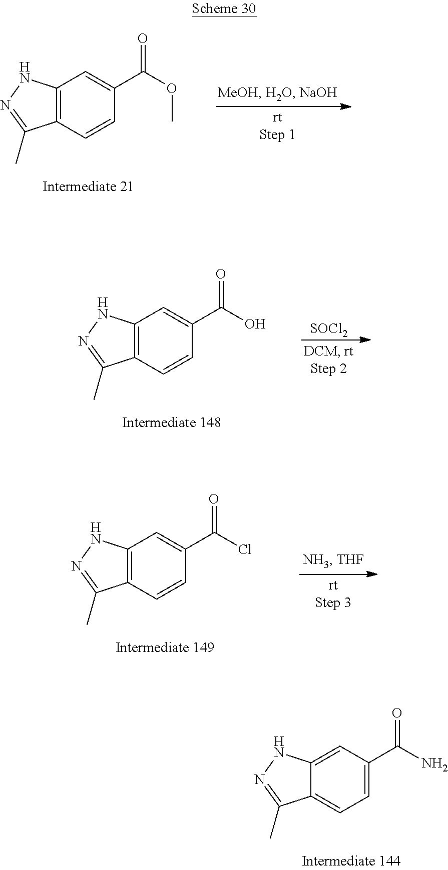

### Step 1

### Intermediate 148: 3-methyl-1H-indazole-6-carboxylic acid

To a solution of methyl 3-methyl-1H-indazole-6-carboxylate (Intermediate 21, 2.00 g, 10.5 mmol) in MeOH (40.0 mL) was added H₂O (40.0 mL) and NaOH (3.20 g, 80.0 mmol). The reaction was stirred at room temperature for 3 h. The pH of the mixture was adjusted to 4-6 by the addition of HCl (1M) and the mixture was extracted with EtOAc (3×200 mL). The combined organic layers were washed with brin and dried over anhydrous Na₂SO₄, filtered and concentrated under reduced pressure to provide 3-methyl-1H-indazole-6-carboxylic acid (Intermediate 148, 1.90 g, 92.4%) as a yellow solid, which was used in the next step without further purification. Mass spec: m/z: 177.0 [M+H]⁺.

### Step 2

### Intermediate 149: 3-methyl-1H-indazole-6-carbonyl chloride

To a solution of 3-methyl-1H-indazole-6-carboxylic acid (Intermediate 148, 1.90 g, 10.8 mmol) in DCM (30.0 mL) was added thionyl chloride (30.0 mL). The reaction was stirred at room temperature overnight and concentrated under reduced pressure to provide 3-methyl-1H-indazole-6-carbonyl chloride (Intermediate 149, 2.00 g, 76.4%) as a white solid, which was used in the next step without further purification. Mass spec: m/z: 195.0 [M+H]⁺.

### Step 3

### Intermediate 144: 3-methyl-1H-indazole-6-carboxamide

To a solution of ammonia (0.5M in THF, 60 mL) was added 3-methyl-1H-indazole-6-carbonyl chloride (Intermediate 149, 2.00 g, 10.3 mmol) at 0° C. and the reaction was stirred at room temperature for 5 h and concentrated under reduced pressure to afford 3-methyl-1H-indazole-6-carboxamide (Intermediate 144, 900 mg, 62.6%) as a white solid. Mass spec: m/z: 176.0 [M+H]⁺.

### Example 31 was Synthesized Following Scheme 31

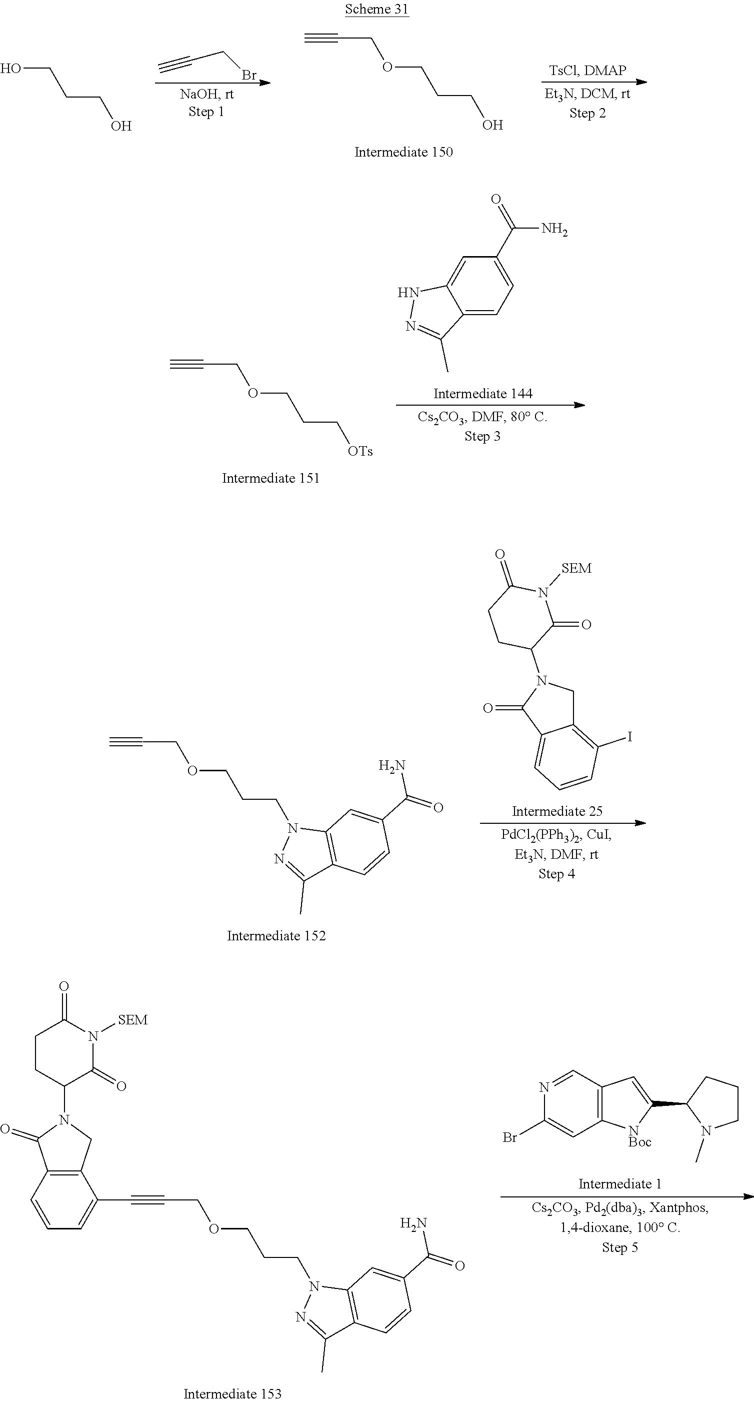

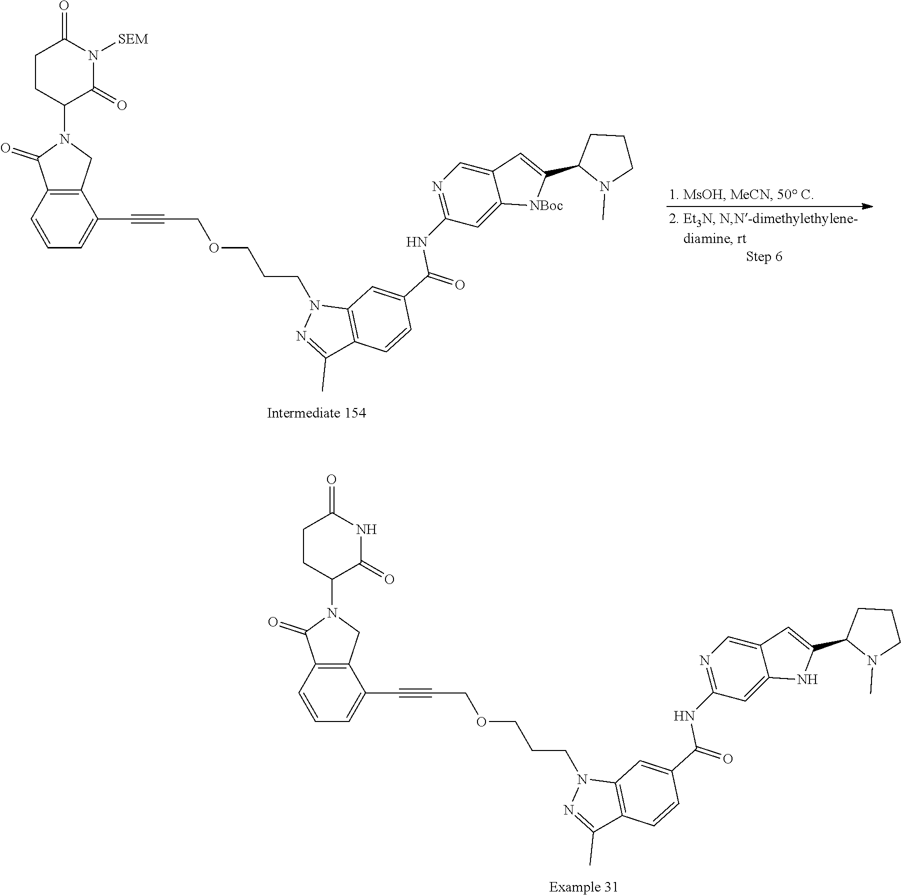 [SMILES: Cc1nn(CCCOCC#Cc2cccc3c2CN(C2CCC(=O)N(COCC[Si](C)(C)C)C2=O)C3=O)c2cc(C(=O)Nc3cc4c(cn3)cc([C@H]3CCCN3C)n4C(=O)OC(C)(C)C)ccc12 ; Cc1nn(CCCOCC#Cc2cccc3c2CN(C2CCC(=O)NC2=O)C3=O)c2cc(C(=O)Nc3cc4[nH]c([C@H]5CCCN5C)cc4cn3)ccc12]

### Step 1

### Intermediate 150: 3-(prop-2-yn-1-yloxy) propan-1-ol

To a mixture of propane-1,3-diol (CAS No: 504-63-2, 102.3 g, 1345.0 mmol) and 3-bromoprop-1-yne (CAS No: 106-96-7, 63.7 mL, 672.49 mmol) was added sodium hydroxide (32.60 g, 806.99 mmol) at 0° C. The reaction mixture was stirred at room temperature for 2h. After completion, the reaction mixture was filtered, washed with dichloromethane (2×100 mL) and the filtrate was concentrated in vacuo. The crude material was purified by combi-flash chromatography, by eluting with 50% ethyl acetate in heptane, to afford 3-(prop-2-yn-1-yloxy) propan-1-ol (Intermediate 150, 30.0 g, 39%) as a pale yellow oil. ¹H NMR (400 MHz, CDCl₃) δ ppm 1.84-1.90 (m, 2H), 2.44 (t, J=2.38 Hz, 1H), 3.71 (t, J=5.54 Hz, 2H), 3.78 (t, J=5.55 Hz, 2H), 4.16 (d, J=2.38 Hz, 2H).

### Step 2

### Intermediate 151: 3-(prop-2-yn-1-yloxy) propyl 4-methylbenzenesulfonate

To a solution of 3-(prop-2-yn-1-yloxy) propan-1-ol (Intermediate 150, 25.0 g, 219.0 mmol) in dichloromethane (250 mL) were added triethylamine (66.49 g, 657.09 mmol) and 4-dimethylaminopyridine (2.82 g, 21.90 mmol) at 0° C. To the reaction mixture p-toluene sulfonyl chloride (62.63 g, 328.54 mmol) was added at 0° C. and stirred at room temperature for 16 h. The reaction mixture was diluted with water (500 mL) and extracted with dichloromethane (3×250 mL). The organic layer was separated, dried over anhydrous Na₂SO₄, and filtered and concentrated in vacuo to afford 3-(prop-2-yn-1-yloxy) propyl 4-methylbenzenesulfonate (Intermediate 151, 37.0 g, 63%) as a yellow liquid, which was used for the next step without further purification. ¹H NMR (400 MHz, DMSO-d₆) δ ppm 1.80-1.82 (m, 2H), 2.42 (s, 3H), 3.35-3.46 (m, 3H), 3.98-4.02 (m, 2H), 4.04-4.06 (m, 2H), 7.48 (d, J=7.20 Hz, 2H), 7.79 (d, J=7.20 Hz, 2H).

### Step 3

### Intermediate 152: 3-methyl-1-(3-(prop-2-yn-1-yloxy)propyl)-1H-indazole-6-carboxamide

To a solution of 3-(prop-2-yn-1-yloxy) propyl 4-methylbenzenesulfonate (Intermediate 151, 4.00 g, 14.91 mmol) in dimethylformamide (50 mL) was added 3-methyl-1H-indazole-6-carboxamide (Intermediate 144, 2.61 g, 14.91 mmol) followed by cesium carbonate (15.34 g, 44.73 mmol) at room temperature. The reaction mixture was heated at 80° C. for 2 h. The reaction mixture was diluted with water (500 mL) and extracted with dichloromethane (3×500 mL). The organic layer was separated, dried over anhydrous Na₂SO₄, filtered, and concentrated in vacuo to obtain the crude compound as a mixture of regioisomers. The regioisomers were separated by combi-flash chromatography, by eluting with 30% ethyl acetate in heptane, to afford the desired regioisomer 3-methyl-1-(3-(prop-2-yn-1-yloxy) propyl)-1H-indazole-6-carboxamide (Intermediate 152, 2.40 g, 59%) as a white solid. ¹H NMR (400 MHz, CDCl₃) δ ppm 2.12-2.24 (m, 2H), 2.34-2.38 (m, 1H), 2.53 (s, 3H), 3.38-3.46 (m, 2H), 4.05-4.12 (m, 2H), 4.42-4.48 (m, 2H), 7.48 (d, J=7.20 Hz, 1H), 7.68 (d, J=7.20 Hz, 1H), 7.96 (s, 1H). Mass spec m/z 272.0 [M+H]⁺.

### Step 4

### Intermediate 153: 1-(3-((3-(2-(2,6-dioxo-1-((2-(trimethylsilyl) ethoxy) methyl) piperidin-3-yl)-1-oxoisoindolin-4-yl) prop-2-yn-1-yl) oxy) propyl)-3-methyl-1H-indazole-6-carboxamide

To a solution of 3-methyl-1-(3-(prop-2-yn-1-yloxy) propyl)-1H-indazole-6-carboxamide (Intermediate 152, 2.40 g, 8.8 mmol) and 3-(4-iodo-1-oxoisoindolin-2-yl)-1-((2-(trimethylsilyl)ethoxy)methyl)piperidine-2,6-dione (Intermediate 25, 4.4 g, 8.8 mmol) in dimethylformamide (30 mL) was added triethylamine (34.0 g, 320.0 mmol). The reaction mixture was purged with argon gas for 15 min then PdCl₂(PPh₃)₂ (0.62 g, 0.88 mmol) and copper (I) iodide (0.17 g, 0.88 mmol) were added. The reaction mixture was purged with argon gas for 10 min and stirred at room temperature for 2 h. The reaction mixture was diluted with water (1000 mL) and extracted with dichloromethane (3×500 mL). The organic layer was separated, dried over anhydrous Na₂SO₄, filtered, and concentrated in vacuo. The crude material was purified by combi-flash chromatography, by eluting with 90% ethyl acetate in heptane, to afford 1-(3-((3-(2-(2,6-dioxo-1-((2-(trimethylsilyl) ethoxy) methyl) piperidin-3-yl)-1-oxoisoindolin-4-yl) prop-2-yn-1-yl) oxy) propyl)-3-methyl-1H-indazole-6-carboxamide (Intermediate 153, 2.00 g, 35%) as a yellow solid. ¹H NMR (400 MHz, DMSO-d₆) δ ppm −0.05 (s, 9H), 0.79-0.84 (m, 2H), 1.98-2.17 (m, 4H), 2.46 (s, 3H), 2.54-2.58 (m, 1H), 2.72-2.78 (m, 1H), 2.99-3.14 (m, 2H), 3.45-3.57 (m, 3H), 4.26-4.30 (m, 1H), 4.38-4.46 (m, 4H), 4.97-5.07 (m, 2H), 5.23-5.28 (m, 1H), 7.39 (br s, 1H), 7.53-7.61 (m, 2H), 7.64 (d, J=7.60 Hz, 1H), 7.68-7.72 (m, 1H), 7.77 (d, J=7.60 Hz, 1H), 8.02 (br s, 1H), 8.12 (s, 1H). Mass spec m/z 644.1 [M+H]⁺.

### Step 5

### Intermediate 154: tert-butyl 6-(1-(3-((3-(2-(2,6-dioxo-1-((2-(trimethylsilyl)ethoxy)methyl)piperidin-3-yl)-1-oxoisoindolin-4-yl)prop-2-yn-1-yl)oxy)propyl)-3-methyl-1H-indazole-6-carboxamido)-2-((R)-1-methylpyrrolidin-2-yl)-1H-pyrrolo[3,2-c]pyridine-1-carboxylate

To a solution of 1-(3-((3-(2-(2,6-dioxo-1-((2-(trimethylsilyl) ethoxy) methyl) piperidin-3-yl)-1-oxoisoindolin-4-yl) prop-2-yn-1-yl) oxy) propyl)-3-methyl-1H-indazole-6-carboxamide (Intermediate 153, 0.67 g, 1.05 mmol) in 1,4-dioxane (15 mL) was added tert-butyl(R)-6-bromo-2-(1-methylpyrrolidin-2-yl)-1H-pyrrolo[3,2-c]pyridine-1-carboxylate (Intermediate 1, 0.50 g, 1.31 mmol) followed by cesium carbonate (1.29 g, 3.94 mmol) at room temperature. The reaction mixture was purged with argon for 15 min followed by Pd₂(dba)₃ (0.18 g, 0.19 mmol) and Xantphos (0.23 g, 0.39 mmol). The reaction mixture was purged with argon for 10 min and heated at 100° C. for 2 h. The reaction mixture was filtered through a celite bed and washed with ethyl acetate (100 mL). The filtrate was concentrated in vacuo. The crude material was purified by combi-flash column chromatography by eluting with 90% ethyl acetate in heptane to afford tert-butyl 6-(1-(3-((3-(2-(2,6-dioxo-1-((2-(trimethylsilyl)ethoxy)methyl)piperidin-3-yl)-1-oxoisoindolin-4-yl)prop-2-yn-1-yl)oxy)propyl)-3-methyl-1H-indazole-6-carboxamido)-2-((R)-1-methylpyrrolidin-2-yl)-1H-pyrrolo[3,2-c]pyridine-1-carboxylate (Intermediate 154, 0.60 g, 48%) as a yellow solid. ¹H NMR (400 MHz, CDCl₃) δ ppm −0.04 (s, 9H), 0.85-0.95 (m, 2H), 1.61-1.71 (m, 2H), 1.76 (s, 9H), 2.05-2.19 (m, 2H), 2.21-2.32 (m, 2H), 2.34-2.40 (m, 2H), 2.43 (s, 3H), 2.57 (s, 3H), 2.79-2.98 (m, 2H), 3.18-3.22 (m, 1H), 3.46-3.50 (m, 2H), 3.55-3.63 (m, 2H), 3.97-4.01 (m, 1H), 4.23-4.41 (m, 4H), 4.50-4.54 (m, 2H), 5.12-5.20 (m, 2H), 5.28 (s, 2H), 6.73 (s, 1H), 7.34-7.38 (m, 1H), 7.46-7.48 (m, 1H), 7.56-7.62 (m, 1H), 7.66-7.70 (m, 1H), 7.75-7.81 (m, 1H), 8.07 (s, 1H), 8.78 (s, 1H), 9.02 (s, 1H). Mass spec m/z 943.2 [M+H]⁺.

### Step 6

### Example 31: 1-(3-((3-(2-(2,6-dioxopiperidin-3-yl)-1-oxoisoindolin-4-yl)prop-2-yn-1-yl)oxy)propyl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(3-((3-(2-(2,6-dioxo-1-((2-(trimethylsilyl)ethoxy)methyl)piperidin-3-yl)-1-oxoisoindolin-4-yl)prop-2-yn-1-yl)oxy)propyl)-3-methyl-1H-indazole-6-carboxamido)-2-((R)-1-methylpyrrolidin-2-yl)-1H-pyrrolo[3,2-c]pyridine-1-carboxylate (Intermediate 154, 0.60 g, 0.63 mmol) in acetonitrile (15 mL) was added methanesulfonic acid (0.42 mL, 6.36 mmol) at room temperature and the reaction was heated at 50° C. for 2 h. The reaction mixture was cooled to room temperature and N,N′-dimethylethylenediamine (0.38 mL, 3.18 mmol) and triethylamine (1.78 mL, 12.72 mmol) were added and the mixture stirred at room temperature for 3 h. The reaction mixture was concentrated in vacuo. The crude residue was quenched with water (50 mL) to form a solid precipitate which was filtered and dried in vacuo. The crude material was purified by preparative HPLC (Method A) to afford 1-(3-((3-(2-(2,6-dioxopiperidin-3-yl)-1-oxoisoindolin-4-yl)prop-2-yn-1-yl)oxy)propyl)-3-methyl-N-(2-((R)-1-methylpyrrolidin-2-yl)-1H-pyrrolo[3,2-c]pyridin-6-yl)-1H-indazole-6-carboxamide (Example 31, 0.12 g, 26%) as an off white solid. ¹H NMR (400 MHz, DMSO-d₆) δ ppm 1.76-2.02 (m, 4H), 2.13-2.16 (m, 2H), 2.18 (s, 3H), 2.19-2.22 (m, 1H), 2.23-2.31 (m, 1H), 2.38-2.42 (m, 2H), 2.52 (s, 3H), 2.81-2.93 (m, 1H), 3.13-3.18 (m, 1H), 3.32-3.37 (m, 1H), 3.54-3.57 (m, 2H), 4.26-4.34 (m, 1H), 4.38-4.40 (m, 1H), 4.43 (s, 2H), 4.44-4.53 (m, 2H), 5.08-5.13 (m, 1H), 6.40 (s, 1H), 7.46-7.54 (m, 1H), 7.60-7.64 (m, 1H), 7.68-7.78 (m, 3H), 8.24 (s, 1H), 8.42 (s, 1H), 8.51 (s, 1H), 10.54 (br s, 1H), 10.97 (br s, 1H), 11.37 (br s, 1H). Mass spec m/z 713.0 [M+H]⁺.

### Example 32 was Synthesized Following Scheme 32

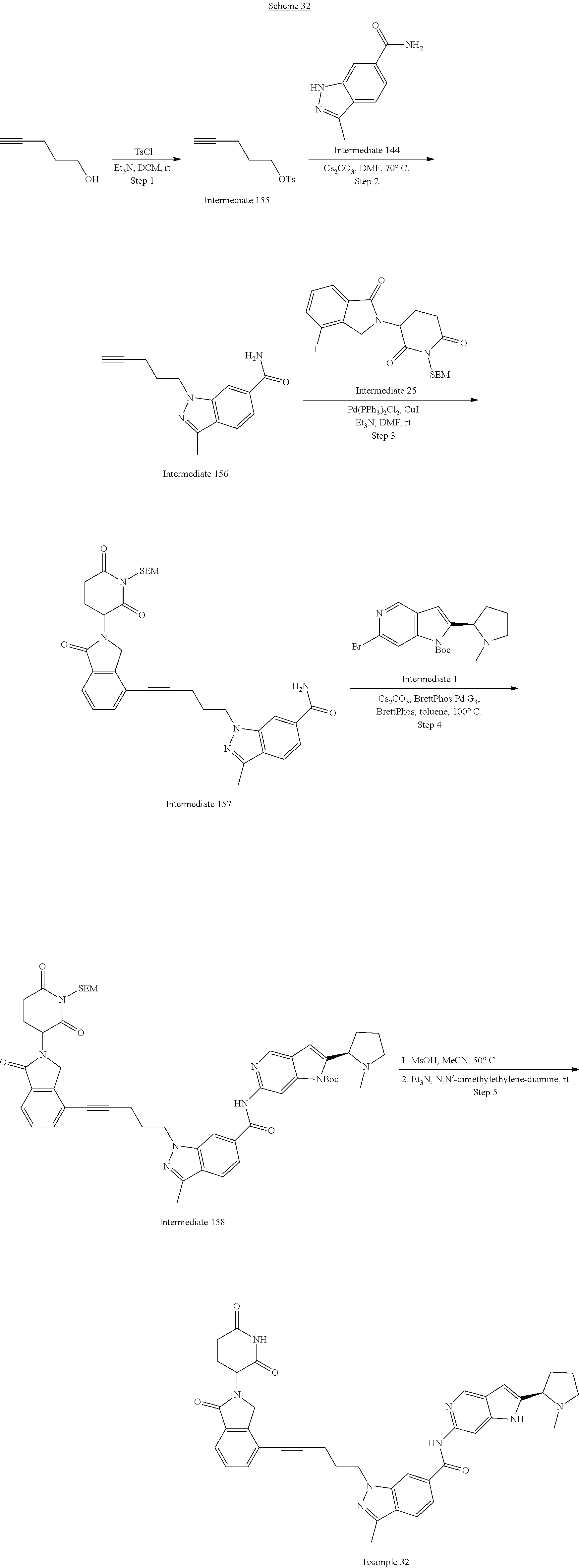

### Step 1

### Intermediate 155: pent-4-yn-1-yl-4-methylbenzenesulfonate

To a cooled (0° C.) solution of pent-4-yn-1-ol (CAS No: 5390-04-5, 5.00 g, 59.4 mmol) in dichloromethane (10 mL) was added triethylamine (16.7 mL, 119.0 mmol), 4-dimethylaminopyridine (0.67 mL, 29.7 mmol) and toluenesulfonyl chloride (17.3 g, 89.2 mmol) and the reaction mixture was stirred at room temperature for 16 h. The reaction mixture was diluted with water (100 mL) and extracted with dichloromethane (3×100 mL). The combined organic layers were washed with NaHCO₃ (2×60 mL) and brine solution (80 mL), dried over anhydrous Na₂SO₄ and concentrated in vacuo. The crude material was purified by combi-flash column chromatography, by eluting with 20% ethyl acetate in heptane, to afford pent-4-yn-1-yl-4-methylbenzenesulfonate (Intermediate 155, 7.5 g, 53%) as a colorless oil. ¹H NMR (400 MHz, DMSO-d₆) δ ppm 1.72-1.75 (m, 2H), 2.15-2.17 (m, 2H), 2.42 (s, 3H), 2.75 (s, 1H), 4.08 (t, J=5.60 Hz, 2H), 7.49 (d, J=7.05 Hz, 2H), 7.79 (d, J=7.46 Hz, 2H).

### Step 2

### Intermediate 156: 3-methyl-1-(pent-4-yn-1-yl)-1H-indazole-6-carboxamide

To a solution of pent-4-yn-1-yl-4-methylbenzenesulfonate (Intermediate 155, 3.00 g, 12.6 mmol) and 3-methyl-1H-indazole-6-carboxamide (Intermediate 144, 2.21 g, 12.6 mmol) in dimethylformamide (50 mL) was added cesium carbonate (12.3 g, 37.8 mmol) and the reaction mixture was heated at 70° C. for 16 h. The reaction mixture was diluted with water (100 mL) and extracted with ethyl acetate (2×100 mL). The combined organic layers were dried over anhydrous Na₂SO₄ and concentrated in vacuo to obtain crude compound as a mixture of regioisomers. The regioisomer were separated by combi-flash chromatography, by eluting with 50-55% ethyl acetate in heptane, to afford desired regioisomer 3-methyl-1-(pent-4-yn-1-yl)-1H-indazole-6-carboxamide (Intermediate 156, 1.6 g, 53%) as a white solid. ¹H NMR (400 MHz, DMSO-d₆) δ ppm 1.98-2.02 (m, 2H), 2.14-2.18 (m, 2H), 2.50 (s, 3H), 2.83 (s, 1H), 4.41 (t, J=6.84 Hz, 2H), 7.41 (br s, 1H), 7.60 (d, J=8.29 Hz, 1H), 7.74 (d, J=8.71 Hz, 1H), 8.03 (br s, 1H), 8.12 (s, 1H). Mass spec: m/z 242.0 [M+H]⁺.

### Step 3

### Intermediate 157: 1-(5-(2-(2,6-dioxo-1-((2-(trimethylsilyl) ethoxy)methyl)piperidin-3-yl)-1-oxoisoindolin-4-yl) pent-4-yn-1-yl)-3-methyl-1H-indazole-6-carboxamide

To a solution of 3-(4-iodo-1-oxoisoindolin-2-yl)-1-((2-(trimethylsilyl)ethoxy)methyl)piperidine-2,6-dione (Intermediate 25, 1.0 g, 4.14 mmol) in dimethylformamide (10 mL) was added copper(J) iodide (0.08 g, 0.41 mmol), PdCl₂(PPh₃)₂ (0.29 g, 0.41 mmol) and 3-methyl-1-(pent-4-yn-1-yl)-1H-indazole-6-carboxamide (Intermediate 156, 2.39 g, 4.14 mmol) followed by triethylamine (20.3 mL, 145.0 mmol). The reaction mixture was purged with argon for 5 min then stirred at room temperature for 2 h. The reaction mixture was diluted with water (60 mL) and extracted with ethyl acetate (3×80 mL). The combined organic layers were dried over anhydrous Na₂SO₄ and concentrated in vacuo. The crude material was purified by combi-flash column chromatography, by eluting with 90% ethyl acetate in heptane, to afford 1-(5-(2-(2,6-dioxo-1-((2-(trimethylsilyl) ethoxy)methyl)piperidin-3-yl)-1-oxoisoindolin-4-yl)pent-4-yn-1-yl)-3-methyl-1H-indazole-6-carboxamide (Intermediate 157, 1.30 g, 51%) as a white solid. ¹H NMR (400 MHz, DMSO-d₆) δ ppm −0.07 (s, 9H), 0.79-0.81 (m, 2H), 2.07-2.15 (m, 3H), 2.38-2.42 (m, 3H), 2.47 (s, 3H), 2.70-2.91 (m, 1H), 2.99-3.08 (m, 1H), 3.47-3.52 (m, 2H), 4.23-4.27 (m, 1H), 4.42 (s, 2H), 4.47-4.51 (m, 1H), 4.99-5.07 (m, 2H), 5.23-5.28 (m, 1H), 7.41 (br s, 1H), 7.50-7.55 (m, 1H), 7.57-7.67 (m, 2H), 7.72-7.74 (m, 2H), 8.02 (br s, 1H), 8.17 (s, 1H). Mass spec: m/z 612.4 [M+H]⁺.

### Step 4

### Intermediate 158: tert-butyl-6-(1-(5-(2-(2,6-dioxo-1-((2-(trimethylsilyl)ethoxy)methyl)piperidin-3-yl)-1-oxoisoindolin-4-yl)pent-4-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(5-(2-(2,6-dioxo-1-((2-(trimethylsilyl)ethoxymethyl)piperidin-3-yl)-1-oxoisoindolin-4-yl)pent-4-yn-1-yl)-3-methyl-1H-indazole-6-carboxamide (Intermediate 157, 0.6 g, 0.97 mmol) and tert-butyl-(R)-6-bromo-2-(1-methylpyrrolidin-2-yl)-1H-pyrrolo[3,2-c]pyridine-1-carboxylate (Intermediate 1, 0.44 g, 1.17 mmol) in toluene (12 mL) was added cesium carbonate (0.95 g, 2.93 mmol). The reaction mixture was purged with argon for 10 min, then BrettPhos (0.08, 0.1466 mmol) and BrettPhos Pd G3 (0.13 g, 0.1466 mmol) were added and the reaction mixture was purged with argon for a further 10 min. The reaction mixture was heated at 100° C. for 3 h then diluted with water (80 mL) and extracted with ethyl acetate (2×80 mL). The combined organic layers were dried over anhydrous Na₂SO₄ and concentrated in vacuo. The crude material was purified by combi-flash column chromatography, by eluting with 2% MeOH in dichloromethane, to afford tert-butyl 6-(1-(5-(2-(2,6-dioxo-1-((2-(trimethylsilyl)ethoxy)methyl)piperidin-3-yl)-1-oxoisoindolin-4-yl)pent-4-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 158, 0.41 g, 46%) as an off white solid. ¹H NMR (400 MHz, DMSO-d₆) δ ppm −0.08 (s, 9H), 0.71-0.79 (m, 2H), 0.81-0.85 (m, 1H), 1.15-1.19 (m, 1H), 1.21-1.23 (m, 1H), 1.62-1.64 (m, 2H), 1.69 (s, 9H), 1.74-1.77 (m, 2H), 1.98-2.03 (m, 2H), 2.15-2.21 (m, 2H), 2.31 (s, 3H), 2.36 (s, 3H), 2.69-2.73 (m, 1H), 2.97-3.06 (m, 1H), 3.11-3.15 (m, 1H), 3.45-3.50 (m, 2H), 3.91-3.94 (m, 1H), 4.20-4.25 (m, 1H), 4.38-4.42 (m, 1H), 4.56 (t, J=6.22 Hz, 2H), 4.95-5.03 (m, 2H), 5.21-5.23 (m, 1H), 6.77 (s, 1H), 7.42-7.49 (m, 1H), 7.60-7.80 (m, 4H), 8.44 (s, 1H), 8.60 (s, 1H), 8.97 (s, 1H), 10.79 (s, 1H). Mass spec: m/z 913.1 [M+H]⁺.

### Step 5

### Example 32: 1-(5-(2-(2,6-dioxopiperidin-3-yl)-1-oxoisoindolin-4-yl)pent-4-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(5-(2-(2,6-dioxo-1-((2-(trimethylsilyl)ethoxy)methyl)piperidin-3-yl)-1-oxoisoindolin-4-yl)pent-4-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 158, 0.38 g, 0.41 mmol) in acetonitrile (10 mL) was added methanesulfonic acid (0.41 mL, 6.24 mmol) and the reaction mixture was heated at 50° C. for 2 h. The reaction mixture was cooled to room temperature and triethylamine (0.87 mL, 6.24 mmol) and N,N′-dimethylethylenediamine (0.29 mL, 2.49 mmol) added, then the mixture stirred at room temperature for 2 h. The reaction mixture was concentrated in vacuo and the crude material was purified by preparative HPLC (Method A) to afford 1-(5-(2-(2,6-dioxopiperidin-3-yl)-1-oxoisoindolin-4-yl)pent-4-yn-1-yl)-3-methyl-N-(2-((R)-1-methylpyrrolidin-2-yl)-1H-pyrrolo[3,2-c]pyridin-6-yl)-1H-indazole-6-carboxamide (Example 32, 0.11 g, 39%) as an off white solid. ¹H NMR (400 MHz, DMSO-d₆) δ ppm 1.79-1.99 (m, 4H), 2.17-2.28 (m, 7H), 2.49 (s, 3H), 2.53 (s, 3H), 2.55-2.57 (m, 1H), 2.82-2.91 (m, 1H), 3.15-3.17 (m, 1H), 3.31-3.34 (m, 1H), 4.25-4.29 (m, 1H), 4.37-4.42 (m, 1H), 4.57 (t, J=6.60 Hz, 2H), 5.08-5.13 (m, 1H), 6.41 (s, 1H), 7.45 (t, J=7.64 Hz, 1H), 7.61 (d, J=7.58, 1H), 7.66 (d, J=7.28 Hz, 1H), 7.70-7.77 (m, 2H), 8.24 (s, 1H), 8.44 (s, 1H), 8.51 (s, 1H), 10.57 (s, 1H), 10.98 (br s, 1H), 11.39 (s, 1H). Mass spec: m/z 683.2 [M+H]⁺.

### Example 33 was Synthesized Following Scheme 33

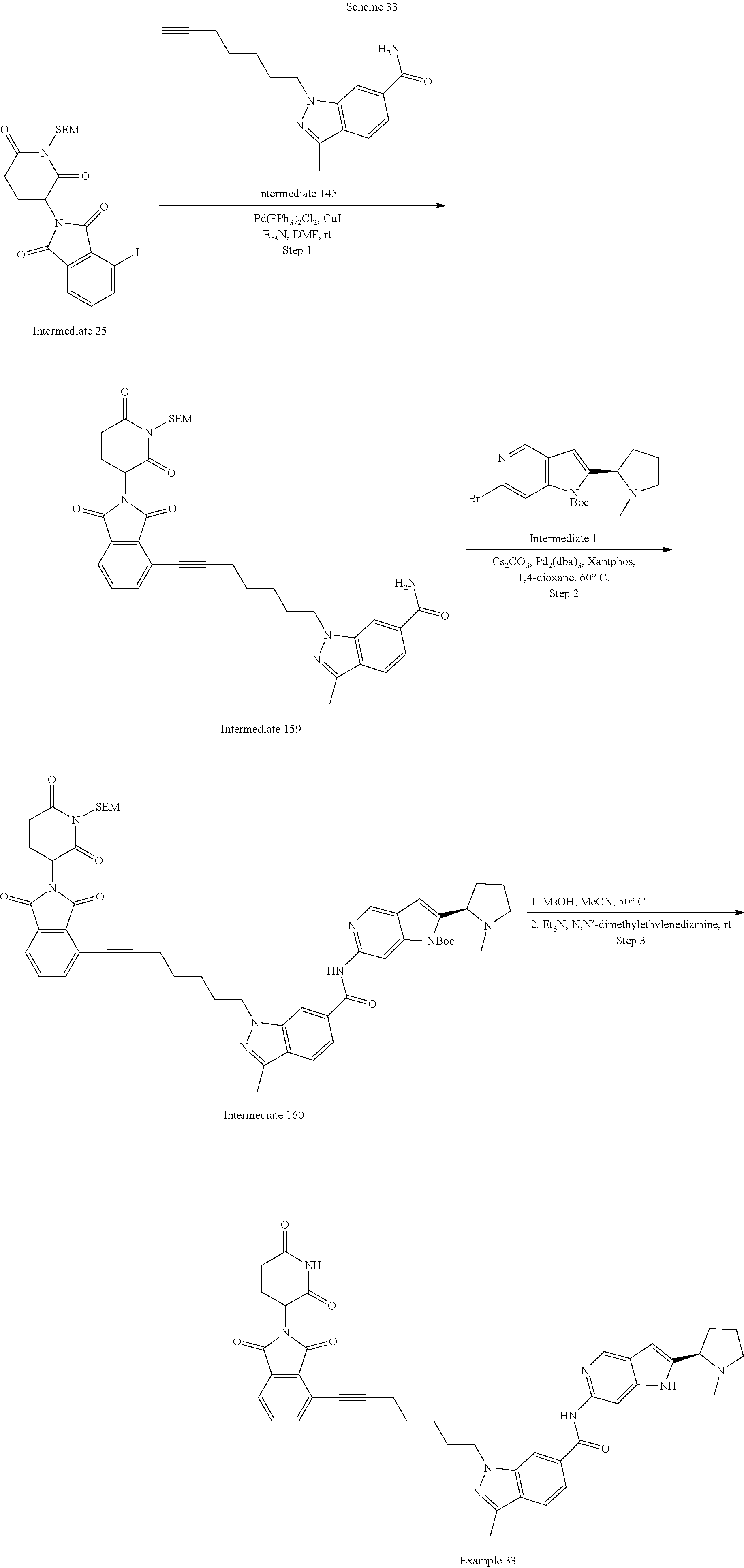 [SMILES: C[Si](C)(C)CCOCN1C(=O)CCC(N2C(=O)c3cccc(I)c3C2=O)C1=O ; C#CCCCCCn1nc(C)c2ccc(C(N)=O)cc21 ; Cc1nn(CCCCCC#Cc2cccc3c2C(=O)N(C2CCC(=O)N(COCC[Si](C)(C)C)C2=O)C3=O)c2cc(C(N)=O)ccc12 ; CN1CCC[C@@H]1c1cc2cnc(Br)cc2n1C(=O)OC(C)(C)C ; Cc1nn(CCCCCC#Cc2cccc3c2C(=O)N(C2CCC(=O)N(COCC[Si](C)(C)C)C2=O)C3=O)c2cc(C(=O)Nc3cc4c(cn3)cc([C@H]3CCCN3C)n4C(=O)OC(C)(C)C)ccc12 ; Cc1nn(CCCCCC#Cc2cccc3c2C(=O)N(C2CCC(=O)NC2=O)C3=O)c2cc(C(=O)Nc3cc4[nH]c([C@H]5CCCN5C)cc4cn3)ccc12]

### Step 1

### Intermediate 159: 1-(7-(2-(2,6-dioxo-1-((2-(trimethylsilyl)ethoxy)methyl)piperidin-3-yl)-1,3-dioxoisoindolin-4-yl)hept-6-yn-1-yl)-3-methyl-1H-indazole-6-carboxamide

To a solution of 2-(2,6-dioxo-1-((2-(trimethylsilyl)ethoxymethyl)piperidin-3-yl)-4-iodoisoindoline-1,3-dione (Intermediate 25, 1.34 g, 2.59 mmol) in dimethylformamide (3 mL) was added 1-(hept-6-yn-1-yl)-3-methyl-1H-indazole-6-carboxamide (Intermediate 145, 0.70 g, 2.59 mmol) followed by triethylamine (9.29 g, 90.98 mmol) and the reaction mixture was purged with argon gas for 10 min. PdCl₂(PPh₃)₂ (0.18 g, 0.26 mmol) and copper (I) iodide (0.05 g, 0.25 mmol) were added and the mixture was stirred at room temperature for 2 h. The reaction mixture was filtered through a celite bed, the filtrate was diluted with water (30 mL) and extracted with ethyl acetate (3×40 mL). The combined organic layers were washed with brine solution (2×50 mL), dried over anhydrous Na₂SO₄ and concentrated in vacuo. The crude material was purified by combi-flash chromatography, by eluting with 65% ethyl acetate in heptane, to obtain 1-(7-(2-(2,6-dioxo-1-((2-(trimethylsilyl)ethoxy)methyl) piperidin-3-yl)-1,3-dioxoisoindolin-4-yl)hept-6-yn-1-yl)-3-methyl-1H-indazole-6-carboxamide (Intermediate 159, 0.75 g, 45%) as a white solid. ¹H NMR (400 MHz, DMSO-d₆) δ ppm −0.04 (s, 9H), 0.80-0.84 (m, 2H), 1.44-1.46 (m, 2H), 1.60-1.62 (m, 2H), 1.86-1.88 (m, 2H), 2.03-2.13 (m, 2H), 2.48 (s, 3H), 2.73-2.89 (m, 2H), 2.95-3.08 (m, 2H), 3.44-3.54 (m, 2H), 4.35-4.37 (m, 2H), 5.06 (s, 2H), 5.25-5.27 (m, 1H), 7.41 (br s, 1H), 7.52-7.66 (m, 2H), 7.72 (d, J=7.89 Hz, 1H), 7.79-7.89 (m, 2H), 8.03 (br s, 1H), 8.14 (s, 1H). Mass spec: m/z 656.4 [M+H]⁺.

### Step 2

### Intermediate 160: tert-butyl-6-(1-(7-(2-(2,6-dioxo-1-((2-(trimethylsilyl)ethoxy)methyl)piperidin-3-yl)-1,3-dioxoisoindolin-4-yl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(7-(2-(2,6-dioxo-1-((2-(trimethylsilyl) ethoxy)methyl)piperidin-3-yl)-1,3-dioxoisoindolin-4-yl) hept-6-yn-1-yl)-3-methyl-1H-indazole-6-carboxamide (Intermediate 159, 0.55 g, 0.84 mmol) and tert-butyl-(R)-6-bromo-2-(1-methylpyrrolidin-2-yl)-1H-pyrrolo[3,2-c]pyridine-1-carboxylate (Intermediate 1, 0.40 g, 1.05 mmol) in 1,4-dioxane (12 mL) was added cesium carbonate (0.68 g, 2.10 mmol) and the reaction mixture was purged with argon gas for 10 min. Pd₂(dba)₃ (0.15 g, 0.15 mmol) and Xantphos (0.18 g, 0.31 mmol) were then added and reaction mixture was purged again with argon gas for 5 min then heated at 60° C. for 2 h. The reaction mixture was diluted with water (70 mL) and extracted with ethyl acetate (3×70 mL). The combined organic layers were washed with brine solution (2×60 mL), dried over anhydrous Na₂SO₄ and concentrated in vacuo. The crude material was purified by combi-flash chromatography, by eluting with 95% ethyl acetate in heptane, to afford tert-butyl-6-(1-(7-(2-(2,6-dioxo-1-((2-(trimethylsilyl)ethoxy)methyl)piperidin-3-yl)-1,3-dioxoisoindolin-4-yl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 160, 0.55 g, 55%) as a liquid. Mass spec: m/z 955.2 [M+H]⁺.

### Step 3

### Example 33: 1-(7-(2-(2,6-dioxopiperidin-3-yl)-1,3-dioxoisoindolin-4-yl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl-6-(1-(7-(2-(2,6-dioxo-1-((2-(trimethylsilyl)ethoxy)methyl)piperidin-3-yl)-1,3-dioxoisoindolin-4-yl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 160, 0.55 g, 0.57 mmol) in acetonitrile (5 mL) was added methanesulfonic acid (0.28 g, 2.87 mmol) and the reaction was heated at 50° C. for 2 h. The reaction mixture was cooled to room temperature then N,N′-dimethylethylenediamine (0.31 g, 3.45 mmol) followed by triethylamine (5.00 g, 49 mmol) were added and the reaction mixture stirred at room temperature for 3 h. The reaction mixture was concentrated in vacuo and the crude material was purified by preparative HPLC (Method A) to afford 1-(7-(2-(2,6-dioxopiperidin-3-yl)-1,3-dioxoisoindolin-4-yl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide (Example 33, 0.10 g, 24%) as an off white solid. ¹H NMR (400 MHz, DMSO-d₆) δ ppm 1.48-1.54 (m, 2H), 1.62-1.69 (m, 2H), 1.79-1.97 (m, 6H), 2.02-2.06 (m, 1H), 2.13-2.16 (m, 1H), 2.18 (s, 3H), 2.24-2.30 (m, 1H), 2.52 (s, 3H), 2.53-2.55 (m, 2H), 2.82-2.91 (m, 1H), 3.13-3.17 (m, 1H), 3.32-3.36 (m, 2H), 4.44 (t, J=6.94 Hz, 2H), 5.09-5.14 (m, 1H), 6.40 (s, 1H), 7.70-7.73 (m, 2H), 7.76-7.80 (m, 2H), 7.82-7.85 (m, 1H), 8.24 (s, 1H), 8.45 (s, 1H), 8.51 (s, 1H), 10.51 (s, 1H), 11.11 (s, 1H), 11.38 (s, 1H). Mass spec: m/z 725.2 [M+H]⁺.

### Example 34 was Synthesized Following Scheme 34

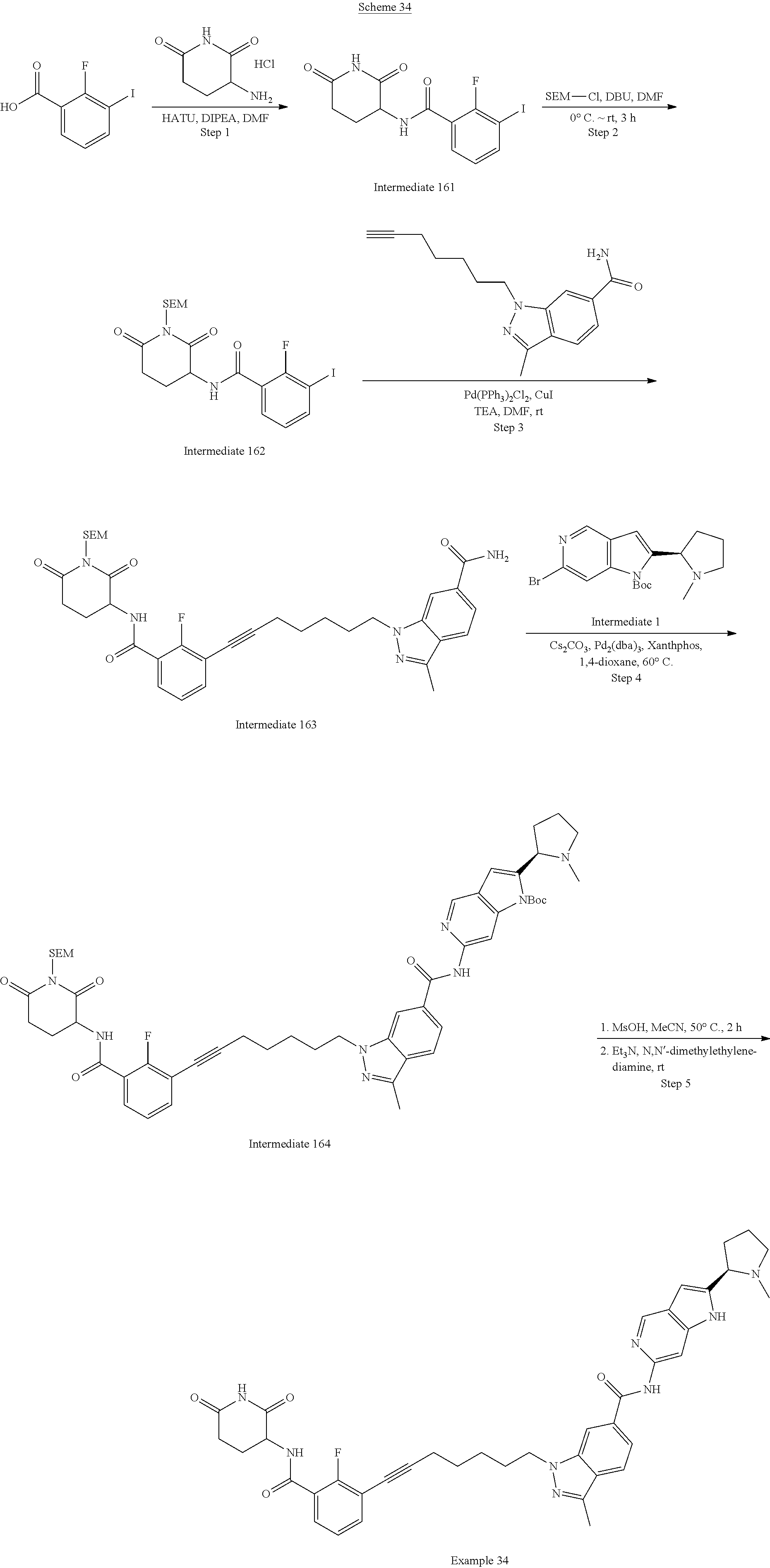

### Step 1

### Intermediate 161: N-(2,6-dioxopiperidin-3-yl)-2-fluoro-3-iodobenzamide

To a solution of 2-fluoro-3-iodobenzoic acid (CAS No: 447464-03-1, 3.0 g, 11.27 mmol) in dimethylformamide (20 mL) were added DIPEA (5.94 g, 45.11 mmol) and HATU (6.77 g, 16.91 mmol). The reaction was stirred at room temperature for 15 min then 3-aminopiperidine-2,6-dione hydrochloride (CAS No: 24666-56-6, 2.22 g, 13.53 mmol) was added and reaction mixture was stirred at room temperature for a further 16 h. The reaction was quenched with water (500 mL) and extracted with EtOAc (3×250 mL). The combined organic layers were over anhydrous Na₂SO₄ and concentrated in vacuo to afford N-(2,6-dioxopiperidin-3-yl)-2-fluoro-3-iodobenzamide (Intermediate 161, 3.90 g) as a yellow solid which was used for the next step without further purification. ¹H NMR (400 MHz, DMSO-d₆) δ ppm 1.97-2.13 (m, 2H), 2.50-2.52 (m, 1H), 2.71-2.88 (m, 1H), 4.70-4.83 (m, 1H), 7.11 (t, J=8.17 Hz, 1H), 7.61 (t, J=8.10 Hz, 1H), 7.98 (t, J=6.63 Hz, 1H), 8.72 (d, J=8.05 Hz, 1H), 10.86 (br s, 1H). Mass spec: m/z 376.8 [M+H]⁺.

### Step 2

### Intermediate 162: N-(2,6-dioxo-1-((2-(trimethylsilyl)ethoxy)methyl)piperidin-3-yl)-2-fluoro-3-iodobenzamide

To a cooled (0° C.) solution of N-(2,6-dioxopiperidin-3-yl)-2-fluoro-3-iodobenzamide (Intermediate 161, 1.50 g, 4 mmol) in DMF (10 mL) was added 1,8-diazabicyclo[5.4.0]undec-7-ene (1.21 g, 8 mmol) and the reaction mixture was stirred for 10 min, followed by the addition of 2-(trimethylsilyl)ethoxymethyl chloride (1.33 g, 8 mmol). The reaction mixture was allowed to warm to room temperature and stirred for 12 h. The reaction was diluted with water (40 mL) and extracted with ethyl acetate (3×50 mL). The combined organic layers were washed with brine (2×50 mL), dried over anhydrous Na₂SO₄, filtered and concentrated in vacuo. The crude material was purified by combi-flash column chromatography, by eluting with 65% ethyl acetate in heptane, to afford N-(2,6-dioxo-1-((2-(trimethylsilyl)ethoxy)methyl)piperidin-3-yl)-2-fluoro-3-iodobenzamide (Intermediate 162, 1.5 g, 74%) as a viscous liquid. ¹H NMR (400 MHz, DMSO-d₆) δ ppm −0.02 (s, 9H), 0.84-0.86 (m, 2H), 2.02-2.12 (m, 2H), 2.70-2.74 (m, 1H), 2.87-3.10 (m, 1H), 3.52-3.54 (m, 2H), 4.87-4.89 (m, 1H), 4.99-5.15 (m, 2H), 7.11-7.12 (m, 1H), 7.61 (s, 1H), 7.92-8.03 (m, 1H), 8.84 (d, J=7.45 Hz, 1H).

### Step 3

### Intermediate 163: 1-(7-(3-((2,6-dioxo-1-((2-(trimethylsilyl)ethoxy)methyl)piperidin-3-yl)carbamoyl)-2-fluorophenyl)hept-6-yn-1-yl)-3-methyl-1H-indazole-6-carboxamide

To a solution of N-(2,6-dioxo-1-((2-(trimethylsilyl)ethoxy)methyl)piperidin-3-yl)-2-fluoro-3-iodobenzamide (Intermediate 162, 1.5 g, 3.71 mmol) and 1-(hept-6-yn-1-yl)-3-methyl-1H-indazole-6-carboxamide Intermediate 145 (0.99 g, 3.71 mmol) in dimethylformamide (10 mL) was added triethylamine (13.28 g, 130.10 mmol), PdCl₂(PPh₃)₂ (0.26 g, 0.27 mmol) and copper (I) iodide (0.07 g, 0.37 mmol). The reaction mixture was stirred at room temperature for 2 h, then diluted with water (100 mL) and extracted with ethyl acetate (3×100 mL). The combined organic layers were dried over anhydrous Na₂SO₄, filtered and concentrated in vacuo. The crude material was purified by combi-flash column chromatography, by eluting with 95% ethyl acetate in heptane, to afford 1-(7-(3-((2,6-dioxo-1-((2-(trimethylsilyl)ethoxy)methyl)piperidin-3-yl)carbamoyl)-2-fluorophenyl)hept-6-yn-1-yl)-3-methyl-1H-indazole-6-carboxamide (Intermediate 163, 1.40 g, 59%) as a yellow sticky liquid. ¹H NMR (400 MHz, DMSO-d₆) δ ppm −0.11-0.06 (m, 9H), 0.79-0.88 (m, 1H), 1.15-1.19 (m, 1H), 1.41-1.43 (m, 2H), 1.56-1.60 (m, 1H), 1.56-1.60 (m, 1H), 1.87-1.90 (m, 2H), 1.98-2.02 (m, 1H), 2.34 (s, 3H), 2.43-2.48 (m, 1H), 2.71-2.75 (m, 1H), 3.17-3.20 (m, 2H), 3.50-3.56 (m, 2H), 4.02-4.08 (m, 1H), 4.35-4.39 (m, 2H), 4.83-4.84 (m, 2H), 5.03-5.10 (m, 1H), 7.19-7.22 (m, 1H), 7.31 (m, 2H), 7.40 (s, 1H), 7.59 (d, J=8.29 Hz, 1H), 7.72 (d, J=8.29 Hz, 1H), 8.02 (s, 1H), 8.14 (s, 1H), 8.71 (d, J=8.29 Hz, 1H).

### Step 4

### Intermediate 164: tert-butyl-6-(1-(7-(3-((2,6-dioxo-1-((2-(trimethylsilyl)ethoxy)methyl)piperidin-3-yl)carbamoyl)-2-fluorophenyl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(7-(3-((2,6-dioxo-1-((2-(trimethylsilyl)ethoxy)methyl)piperidin-3-yl)carbamoyl)-2-fluorophenyl)hept-6-yn-1-yl)-3-methyl-1H-indazole-6-carboxamide (Intermediate 163, 1.36 g, 2.10 mmol) and tert-butyl-(R)-6-bromo-2-(1-methylpyrrolidin-2-yl)-1H-pyrrolo[3,2-c]pyridine-1-carboxylate (Intermediate 1, 1.0 g, 2.63 mmol) in 1,4-dioxane (30 mL) was added cesium carbonate (1.72 g, 5.25 mmol). The mixture was purged with argon gas for 20 min, then Xantphos (0.47 g, 0.78 mmol) and Pd₂(dba)₃ (0.37 g, 0.39 mmol) were added and the reaction mixture was stirred at 60° C. for 2 h. The reaction mixture was diluted with water (70 mL) and extracted with ethyl acetate (3×70 mL). The combined organic layers were washed with brine (2×60 mL), dried over anhydrous Na₂SO₄ and concentrated in vacuo. The crude material was purified by combi-flash column chromatography, by eluting with 95% ethyl acetate in heptane, to afford tert-butyl-6-(1-(7-(3-((2,6-dioxo-1-((2-(trimethylsilyl)ethoxy)methyl)piperidin-3-yl)carbamoyl)-2-fluorophenyl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 164, 0.55 g, 22%) as an off-white solid. ¹H NMR (400 MHz, DMSO-d₆) δ ppm −0.14-0.01 (m, 9H), 0.72-0.90 (m, 2H), 1.06-1.29 (m, 2H), 1.36-1.53 (m, 2H), 1.57-1.79 (m, 14H), 1.70 (s, 3H), 1.87-1.96 (m, 4H), 2.31-2.44 (m, 4H), 2.28 (s, 3H), 3.13-3.22 (m, 1H), 3.27-3.29 (m, 2H), 3.42-3.61 (m, 1H), 3.88-3.97 (m, 1H), 4.03 (m, 1H), 4.34-4.55 (m, 2H), 4.97-5.12 (m, 1H), 6.76 (s, 1H), 7.13-7.45 (m, 1H), 7.16-7.30 (m, 1H), 7.30-7.41 (m, 1H), 7.44 (d, J=8.88 Hz, 1H), 7.47-7.52 (m, 1H) 7.54-7.59 (m, 1H), 7.69-7.74 (m, 1H), 7.74-7.81 (m, 1H), 8.61 (s, 1H), 8.95 (br s, 1H).

### Step 5

### Example 34: 1-(7-(3-((2,6-dioxopiperidin-3-yl)carbamoyl)-2-fluorophenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl-6-(1-(7-(3-((2,6-dioxo-1-((2-(trimethylsilyl)ethoxy)methyl)piperidin-3-yl)carbamoyl)-2-fluorophenyl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 164, 0.55 g, 0.58 mmol) in acetonitrile (10 mL) was added methanesulfonic acid (0.56 g, 5.80 mmol) and the mixture heated at 50° C. for 2 h. The reaction mixture was then cooled to room temperature, then N,N′-dimethylethylenediamine (0.28 g, 2.90 mmol) followed by triethylamine (1.18 g, 11.61 mmol) were added and the reaction was stirred at room temperature for 3 h. The reaction was concentrated in vacuo and the crude compound was purified by preparative HPLC (Method A) to afford 1-(7-(3-((2,6-dioxopiperidin-3-yl)carbamoyl)-2-fluorophenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide (Example 34, 0.095 g, 23%) as an off-white solid. 400 MHz, DMSO-d₆) δ ppm 1.41-1.52 (m, 2H), 1.62-1.67 (m, 2H), 1.78-1.96 (m, 5H), 1.98-2.10 (m, 2H), 2.12-2.16 (m, 1H), 2.18 (s, 3H), 2.22-2.32 (m, 1H), 2.45-2.48 (m, 3H), 2.49 (s, 3H), 2.68-2.87 (m, 2H), 3.15 (t, J=8.13 Hz, 1H), 4.43-4.45 (m, 2H), 4.72-4.80 (m, 1H), 6.40 (s, 1H), 7.23 (t, J=7.69 Hz, 1H), 7.47-7.58 (m, 2H), 7.73 (d, J=8.17 Hz, 1H), 7.78 (d, J=8.65 Hz, 1H), 8.25 (s, 1H), 8.45 (s, 1H), 8.52 (s, 1H), 8.67 (d, J=7.88 Hz, 1H), 10.53 (s, 1H), 10.86 (s, 1H), 11.38 (s, 1H). Mass spec: m/z 717.2 [M+H]⁺.

### Example 35 was Synthesized Following Scheme 35

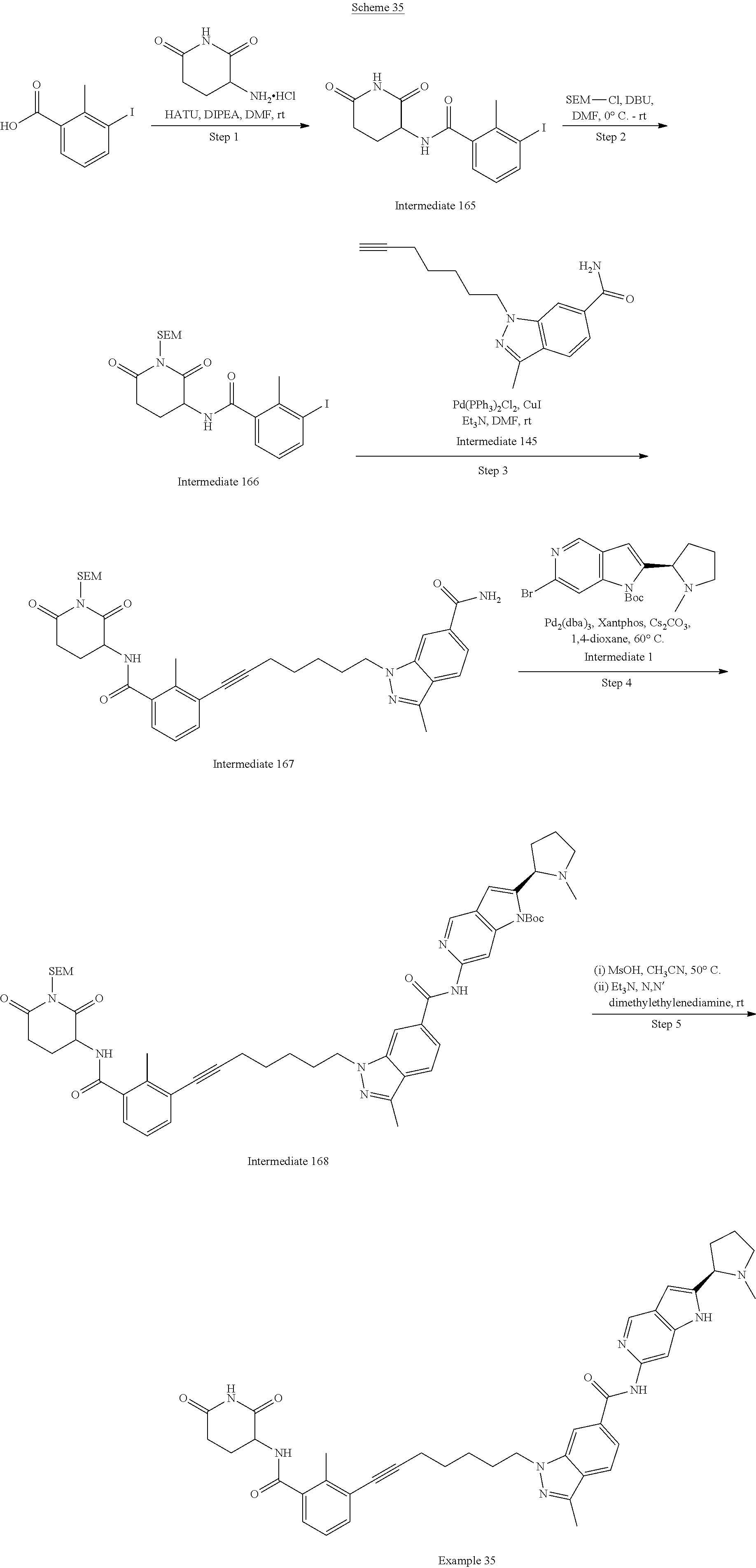

### Step 1

### Intermediate 165: N-(2,6-dioxopiperidin-3-yl)-3-iodo-2-methylbenzamide

To a solution of 3-iodo-2-methyl-benzoic acid (CAS No. 133232-56-1, 5.0 g, 19.08 mmol) and 3-aminopiperidine-2,6-dione hydrochloride (CAS No: 24666-56-6, 3.14 g, 19.08 mmol) in dimethylformamide (50 mL) was added HATU (11.45 g, 28.62 mmol) and DIPEA (7.55 g, 57.24 mmol) and the reaction mixture was stirred at room temperature for 16 h. The reaction mixture was diluted with water (50 mL) and extracted with ethyl acetate (3×100 mL). The combined organic layers were washed with brine, dried over anhydrous Na₂SO₄ and concentrated in vacuo to afford N-(2,6-dioxopiperidin-3-yl)-3-iodo-2-methylbenzamide (Intermediate 165, 5.0 g, crude) as a white solid which was used for the next step without further purification. ¹H NMR (400 MHz, DMSO-d₆) δ ppm 2.01-2.08 (m, 2H), 2.43 (s, 3H), 2.51-2.55 (m, 1H), 2.73-2.76 (m, 1H), 4.68-4.74 (m, 1H), 7.02 (t, J=7.2 Hz, 1H). 7.33 (d, J=7.2 Hz, 1H), 7.92 (d, J=7.60 Hz, 1H), 8.67 (d, J=8.4 Hz, 1H), 10.84 (s, 1H). Mass spec: m/z 372.8 [M+H]⁺.

### Step 2

### Intermediate 166: N-(2,6-dioxo-1-((2-(trimethylsilyl)ethoxy)methyl)piperidin-3-yl)-3-iodo-2-methylbenzamide

To a cooled (0° C.) solution of N-(2,6-dioxo-3-piperidyl)-3-iodo-5-methylbenzamide (Intermediate 165, 2.5 g, 6.7 mmol) and 1,8-diazabicyclo[5.4.0]undec-7-ene (2.10 g, 13 mmol) in dimethylformamide (10 mL) was added 2-(trimethylsilyl)ethoxymethyl chloride (2.40 g, 13 mmol) and the reaction mixture was stirred at room temperature for 2 h. The mixture was diluted with water (50 mL) and extracted with ethyl acetate (3×60 mL). The combined organic layers were washed with brine (2×60 mL), dried over anhydrous Na₂SO₄ and concentrated in vacuo. The crude material was purified by combi-flash column chromatography, by eluting with 60-65% ethyl acetate in hexane, to afford N-(2,6-dioxo-1-((2-(trimethylsilyl)ethoxy)methyl)piperidin-3-yl)-3-iodo-2-methylbenzamide (Intermediate 166, 2.70 g, 80%) as a light yellow sticky compound. ¹H NMR (500 MHz, DMSO-d₆) δ ppm −0.18 (s, 9H), 0.84 (t, J=7.6 Hz, 2H), 1.99-2.11 (m, 2H), 2.45 (s, 3H), 2.71-2.75 (m, 1H), 2.89-2.95 (m, 1H), 3.52-3.55 (m, 2H), 4.82-4.87 (m, 1H), 5.03-5.09 (m, 2H), 7.03 (t, J=8.0 Hz, 1H), 7.35 (d, J=7.6 Hz, 1H), 7.93 (d, J=8.0 Hz, 1H), 8.79 (d, J=8.4 Hz, 1H). Mass spec: m/z 500.9 [M−H]⁻.

### Step 3

### Intermediate 167: 1-(7-(3-((2,6-dioxo-1-((2-(trimethylsilyl)ethoxy)methyl)piperidin-3-yl)carbamoyl)-2-methylphenyl)hept-6-yn-1-yl)-3-methyl-1H-indazole-6-carboxamide

To a solution of N-(2,6-dioxo-1-((2-(trimethylsilyl)ethoxy)methyl)piperidin-3-yl)-3-iodo-2-methylbenzamide (Intermediate 166, 1.87 g, 3.71 mmol) and 1-hept-6-ynyl-3-methyl-indazole-6-carboxamide (Intermediate 145, 1.0 g, 3.71 mmol) in dimethylformamide (10 mL) was added triethylamine (13.28 g, 130.0 mmol) and the reaction mixture was purged with argon for 5 min. Pd(PPh₃)Cl₂ (266 mg, 0.37 mmol) and copper(J) iodide (72 mg, 0.37 mmol) were then added and the reaction mixture was purged with argon for a further 5 min, then stirred at room temperature for 2 h. The, mixture was filtered through a celite bed and the filtrate was diluted with water (100 mL) and extracted with ethyl acetate (3×100 mL). The combined organic layers were washed with brine (2×60 mL), dried over anhydrous Na₂SO₄ and concentrated in vacuo. The crude product was purified by combi-flash column chromatography, by eluting with 90% ethyl acetate in heptane, to afford 1-(7-(3-((2,6-dioxo-1-((2-(trimethylsilyl)ethoxy)methyl)piperidin-3-yl)carbamoyl)-2-methylphenyl)hept-6-yn-1-yl)-3-methyl-1H-indazole-6-carboxamide (Intermediate 167, 1.4 g, 59%) as a yellow sticky solid. ¹H NMR (500 MHz, DMSO-d₆) δ ppm −0.24 (s, 9H), 0.83 (t, J=8.4 Hz, 2H), 1.18 (t, J=6.80 Hz, 1H), 1.41-1.43 (m, 2H), 1.56-160 (m, 2H), 1.88 (t, J=7.60 Hz, 2H), 2.00 (s, 3H), 2.34 (s, 3H), 2.43-2.47 (m, 1H), 2.71-2.76 (m, 1H), 2.91-2.95 (m, 1H), 3.06-3.17 (m, 1H), 3.51-3.56 (m, 2H), 4.02-4.09 (m, 1H), 4.37 (t, J=6.80 Hz, 2H), 4.60-4.83 (m, 1H), 5.03-5.07 (m, 2H), 7.21 (t, J=7.2 Hz, 1H), 7.28-7.33 (m, 2H), 7.40 (br s, 1H), 7.58 (d, J=8.0 Hz, 1H), 7.72 (d, J=8.0 Hz, 1H), 8.02 (br s, 1H), 8.14 (s, 1H), 8.70 (d, J=8.40 Hz, 1H).

### Step 4

### Intermediate 168: tert-butyl-6-(1-(7-(3-((2,6-dioxo-1-((2-(trimethylsilyl)ethoxy)methyl)piperidin-3-yl)carbamoyl)-2-methylphenyl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(7-(3-((2,6-dioxo-1-((2-(trimethylsilyl)ethoxy)methyl)piperidin-3-yl)carbamoyl)-2-methylphenyl)hept-6-yn-1-yl)-3-methyl-1H-indazole-6-carboxamide (Intermediate 167, 1.35 g, 2.10 mmol) and tert-butyl-(R)-6-bromo-2-(1-methylpyrrolidin-2-yl)-1H-pyrrolo[3,2-c]pyridine-1-carboxylate (Intermediate 1, 1.0 g, 2.63 mmol) in 1,4-dioxane (40 mL) was added cesium carbonate (1.72 g, 5.29 mmol). The resulting mixture was degassed with argon for 20 min, then Pd₂(dba)₃ (370 mg, 0.39 mmol) and Xantphos (470 mg, 0.78 mmol) were added and the reaction mixture was degassed again with argon for 10 min, then heated at 60° C. for 2 h. The mixture was diluted with water (70 mL) and extracted with ethyl acetate (3×70 mL). The combined organic layers were washed with brine (2×60 mL), dried over anhydrous Na₂SO₄ and concentrated in vacuo. The crude product was purified by combi-flash column chromatography, by eluting with 90% ethyl acetate in hexane, to afford tert-butyl-6-(1-(7-(3-((2,6-dioxo-1-((2-(trimethylsilyl)ethoxy)methyl)piperidin-3-yl)carbamoyl)-2-methylphenyl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 168, 550 mg, 22%) as a white solid. ¹H NMR (400 MHz, DMSO-d₆) δ ppm −0.37 (s, 9H), 0.82 (t, J=8.0 Hz, 2H), 1.08 (t, J=6.8 Hz, 1H), 1.36-1.46 (m, 2H), 1.61-1.65 (m, 4H), 1.70 (s, 9H), 1.71-1.75 (m, 1H), 2.01 (s, 3H), 2.09-2.14 (m, 2H), 2.24-2.27 (m, 1H), 2.28 (s, 3H), 2.34 (s, 3H), 2.67-2.75 (m, 2H), 2.91-2.94 (m, 2H), 3.13-3.25 (m, 2H), 3.37-3.39 (m, 1H), 3.53-3.55 (m, 2H), 3.93 (t, J=7.60 Hz, 1H), 4.43-4.49 (m, 2H), 4.70-4.83 (m, 2H), 5.03-5.09 (m, 1H), 6.76 (s, 1H), 7.19 (t, J=6.0 HZ, 1H), 7.27 (d, J=7.60 Hz, 1H), 7.33 (d, J=7.20 Hz, 1H), 7.72 (d, J=8.0 Hz, 1H), 7.78 (d, J=8.40 Hz, 1H), 8.45 (s, 1H), 8.61 (s, 1H), 8.69 (d, J=8.0 Hz, 1H), 8.97 (s, 1H), 10.74 (s, 1H). Mass spec: m/z 943.5 [M+H]⁺.

### Step 5

### Example 35: 1-(7-(3-((2,6-dioxopiperidin-3-yl)carbamoyl)-2-methylphenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(7-(3-((2,6-dioxo-1-((2-(trimethylsilyl)ethoxy)methyl)piperidin-3-yl)carbamoyl)-2-methylphenyl)hept-6-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 168, 500 mg, 0.53 mmol) in acetonitrile (10 mL) was added methanesulfonic acid (519 mg, 5.30 mmol) and the reaction mixture was heated at 50° C. for 1 h. After cooling to room temperature, N,N′-dimethylethylenediamine (259 mg, 2.65 mmol) followed by triethylamine (1.08 g, 10.60 mmol) were added and the reaction mixture was stirred at room temperature for 3 h. The mixture was concentrated in vacuo to one third volume and then poured into ice-cold water (20 mL). The resultant precipitate was collected by filtration and dried in vacuo. The solid was purified by preparative HPLC (Method A) to afford 1-(7-(3-((2,6-dioxopiperidin-3-yl)carbamoyl)-2-methylphenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide (Example 35, 65 mg, 15%) as an off-white solid. ¹H NMR (400 MHz, DMSO-d₆) δ ppm 1.45-1.59 (m, 2H), 1.61-1.78 (m, 2H), 1.86-1.91 (m, 9H), 2.17 (s, 3H), 2.28-2.31 (m, 1H), 2.46 (s, 3H), 2.52 (s, 3H), 2.53-2.56 (m, 1H), 2.74-2.78 (m, 1H), 3.15 (t, J=7.20 Hz, 1H), 3.31-3.35 (m, 1H), 3.36-3.45 (m, 1H), 4.40 (t, J=6.80 Hz, 2H), 4.69-4.74 (m, 1H), 6.40 (s, 1H), 7.18 (t, J=7.60 Hz, 1H), 7.26 (d, J=8.0 Hz, 1H), 7.31-7.34 (m, 1H), 7.75-7.79 (m, 2H), 8.25 (s, 1H), 8.45 (s, 1H), 8.52 (s, 1H), 8.58 (d, J=8.4 Hz, 1H), 10.50 (s, 1H), 10.85 (s, 1H), 11.4 (s, 1H). Mass spec: m/z 713.2 [M+H]⁺.

### Example 36 was Prepared Following Scheme 36

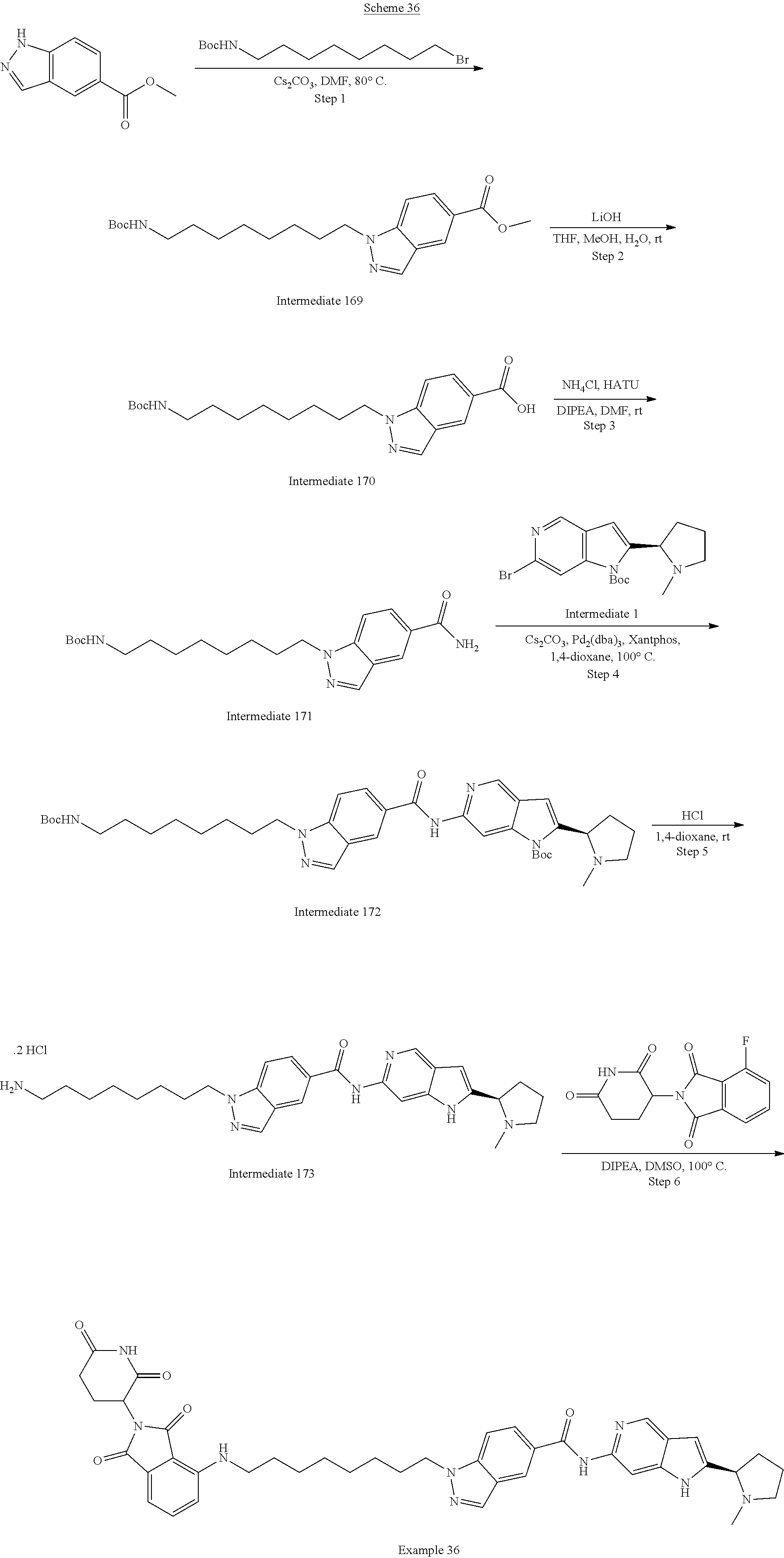

### Step 1

### Intermediate 169: methyl-1-(8-((tert-butoxycarbonyl)amino)octyl)-1H-indazole-5-carboxylate

To solution of methyl 1H-indazole-5-carboxylate (CAS No: 473416-12-5, 2.00 g, 11.35 mmol) and tert-butyl N-(8-bromooctyl)carbamate (CAS No: 142356-35-2, 4.20 g, 13.62 mmol) in dimethylformamide (25 mL) was added cesium carbonate (11.1 g, 34.05 mmol) and the reaction mixture was heated at 80° C. for 2 h. The reaction mixture was then diluted with water (100 mL) and extracted with ethyl acetate (2×50 mL). The combined organic layers were dried over anhydrous Na₂SO₄ and concentrated in vacuo. The crude material was purified by combi-flash chromatography, by eluting with 20-25% ethyl acetate in heptane, to afford the desired regioisomer methyl-1-(8-((tert-butoxycarbonyl)amino)octyl)-1H-indazole-5-carboxylate (Intermediate 169, 2.5 g, 55%) as a brown solid. ¹H NMR (400 MHz, DMSO-d₆) δ ppm 1.17-1.31 (m, 9H), 1.35 (s, 9H), 1.80-1.84 (m, 2H), 2.83-2.88 (m, 2H), 3.16-3.18 (m, 1H), 3.87 (s, 3H), 4.43 (t, J=6.63 Hz, 2H), 6.71 (br s, 1H), 7.76 (d, J=8.71 Hz, 1H), 7.94 (d, J=8.71 Hz, 1H), 8.25 (s, 1H), 8.47 (s, 1H). Mass spec: m/z 404.3 [M+H]⁺.

### Step 2

### Intermediate 170: 1-(8-((tert-butoxycarbonyl)amino)octyl)-1H-indazole-5-carboxylic acid

To a solution of methyl-1-(8-((tert-butoxycarbonyl)amino)octyl)-1H-indazole-5-carboxylate (Intermediate 69, 2.50 g, 6.20 mmol) in THF (20 mL), methanol (20 mL) and H₂O (10 mL) was added LiOH (0.59 g, 25 mmol) and the reaction mixture was stirred at room temperature for 16 h. The reaction mixture was concentrated in vacuo, water (10 mL) was added, and the pH of the solution was adjusted to pH~4 by the addition of saturated aqueous citric acid solution. The resultant precipitate was collected by filtration, washed with water (30 mL) and dried in vacuo to afford 1-(8-((tert-butoxycarbonyl)amino)octyl)-1H-indazole-5-carboxylic acid (Intermediate 170, 2.2 g, 91%) as a yellow solid. ¹H NMR (400 MHz, DMSO-d₆) δ ppm 1.18-1.32 (m, 10H), 1.36 (s, 9H), 1.78-1.82 (m, 2H), 2.83-2.86 (m, 2H), 4.35 (t, J=6.63 Hz, 2H), 6.72 (br s, 1H), 7.47 (d, J=8.71 Hz, 1H), 7.96 (d, J=8.71 Hz, 1H), 8.04 (s, 1H), 8.25 (s, 1H). Mass spec: m/z 390.2 [M+H]⁺.

### Step 3

### Intermediate 171: tert-butyl-(8-(5-carbamoyl-1H-indazol-1-yl) octyl) carbamate

To a solution of 1-[8-(tert-butoxycarbonylamino)octyl]indazole-5-carboxylic acid (Intermediate 170, 2.20 g, 5.6 mmol) and ammonium chloride (1.5 g, 28 mmol) in dimethylformamide (5 mL) was added HATU (3.3 g, 8.5 mmol) followed by DIPEA (5 mL, 28 mmol) and the reaction mixture was stirred at room temperature for 16 h. The reaction mixture was then poured into water (150 mL) and the resulting precipitate was collected by filtration, washed with water (50 mL) and dried in vacuo to afford tert-butyl-(8-(5-carbamoyl-1H-indazol-1-yl) octyl) carbamate (Intermediate 171, 1.70 g, 77%) as a yellow solid. ¹H NMR (400 MHz, DMSO-d₆) δ ppm 1.19-1.31 (m, 10H), 1.35 (s, 9H), 1.80-1.83 (m, 2H), 2.83-2.86 (m, 2H), 4.41 (t, J=6.84 Hz, 2H), 6.72 (br s, 1H), 7.26 (br s, 1H), 7.69 (d, J=8.71 Hz, 1H), 7.89 (d, J=8.71 Hz, 1H), 7.96 (br s, 1H), 8.17 (s, 1H), 8.33 (s, 1H). Mass spec: m/z 389.2 [M+H]⁺.

### Step 4

### Intermediate 172: tert-butyl-(R)-6-(1-(8-((tert-butoxycarbonyl)amino)octyl)-1H-indazole-5-carboxamido)-2-(1-methylpyrrolidin-2-yl)-1H-pyrrolo[3,2-c]pyridine-1-carboxylate

A solution of tert-butyl-(8-(5-carbamoyl-1H-indazol-1-yl) octyl) carbamate (Intermediate 171, 0.50 g, 1.28 mmol), tert-butyl-(R)-6-bromo-2-(1-methylpyrrolidin-2-yl)-1H-pyrrolo[3,2-c]pyridine-1-carboxylate (Intermediate 1, 0.58 g, 1.54 mmol) and cesium carbonate (1.27 g, 3.86 mmol) in 1,4-dioxane (10.0 mL) was purged with argon gas for 30 min. Pd₂(dba)₃ (0.18 g, 0.19 mmol) and Xantphos (0.23 g, 0.38 mmol) were added and the reaction was purged further with argon for 10 min and then heated at 100° C. for 2 h. The reaction mixture was then concentrated in vacuo. The crude material was purified by combi-flash column chromatography, by eluting with 90% ethyl acetate in heptane, to afford tert-butyl-(R)-6-(1-(8-((tert-butoxycarbonyl)amino)octyl)-1H-indazole-5-carboxamido)-2-(1-methylpyrrolidin-2-yl)-1H-pyrrolo[3,2-c]pyridine-1-carboxylate (Intermediate 172, 0.44 g, 50%) as a yellow solid. ¹H NMR (400 MHz, DMSO-d₆) δ ppm 1.14-1.29 (m, 10H), 1.35 (s, 9H), 1.70 (s, 9H), 1.74-1.76 (m, 2H), 1.82-1.85 (m, 3H), 2.31-2.33 (m, 1H), 2.36 (s, 3H), 2.36-2.38 (m, 1H), 2.84-2.89 (m, 2H), 3.11-3.15 (m, 1H), 3.91-3.95 (m, 1H), 4.44 (t, J=6.85 Hz, 2H), 6.71 (br s, 1H), 6.75 (s, 1H), 7.76 (d, J=8.80 Hz, 1H), 8.06 (d, J=8.80 Hz, 1H), 8.22 (s, 1H), 8.56 (s, 1H), 8.60 (s, 1H), 8.95 (s, 1H), 10.67 (s, 1H). Mass spec: m/z 688.5 [M+H]⁺.

### Step 5

### Intermediate 173: (R)-1-(8-aminooctyl)-N-(2-(1-methylpyrrolidin-2-yl)-1H-pyrrolo[3,2-c]pyridin-6-yl)-1H-indazole-5-carboxamide dihydrochloride

To a cooled (0° C.) solution of tert-butyl-(R)-6-(1-(8-((tert-butoxycarbonyl)amino)octyl)-1H-indazole-5-carboxamido)-2-(1-methylpyrrolidin-2-yl)-1H-pyrrolo[3,2-c]pyridine-1-carboxylate (Intermediate 172, 0.44 g, 0.63 mmol) in 1,4-dioxane (4 mL) was added 4M HCl in 1,4-dioxane (8 mL, 32 mmol) and the reaction mixture was stirred at room temperature for 16 h. The reaction mixture was then concentrated in vacuo to afford (R)-1-(8-aminooctyl)-N-(2-(1-methylpyrrolidin-2-yl)-1H-pyrrolo[3,2-c]pyridin-6-yl)-1H-indazole-5-carboxamide dihydrochloride (Intermediate 173, 0.38 g, crude) as a brown solid which was used in the next step without further purification. ¹H NMR (400 MHz, DMSO-d₆) δ ppm 1.19-1.29 (m, 10H), 1.48-1.52 (m, 2H), 1.85-1.92 (m, 2H), 2.18-2.21 (m, 2H), 2.72 (s, 3H), 2.80-2.82 (m, 2H), 3.65-3.73 (m, 2H), 4.46-4.49 (m, 1H), 4.70-4.73 (m, 2H), 7.24 (s, 1H), 7.81 (s, 3H), 7.87 (d, J=8.71 Hz, 1H), 8.17 (d, J=8.71 Hz, 1H), 8.25 (s, 1H), 8.32 (s, 1H), 8.73 (s, 1H), 9.05 (br s, 1H), 11.30 (br s, 1H), 11.89 (br s, 1H), 13.11 (br s, 1H).

### Step 6

### Example 36: 1-(8-((2-(2,6-dioxopiperidin-3-yl)-1,3-dioxoisoindolin-4-yl)amino)octyl)-N-(2-((R)-1-methylpyrrolidin-2-yl)-1H-pyrrolo[3,2-c]pyridin-6-yl)-1H-indazole-5-carboxamide

To a solution of (R)-1-(8-aminooctyl)-N-(2-(1-methylpyrrolidin-2-yl)-1H-pyrrolo[3,2-c]pyridin-6-yl)-1H-indazole-5-carboxamide dihydrochloride (Intermediate 173, 0.35 g, 0.66 mmol) in DMSO (3 mL) was added DIPEA (0.58 mL, 3.34 mmol) followed by 2-(2,6-dioxopiperidin-3-yl)-4-fluoroisoindoline-1,3-dione (CAS No: 835616-60-9, 0.18 g, 0.66 mmol) and the reaction mixture was heated at 100° C. for 2 h. The reaction mixture was then quenched with ice cold water (50 mL) and the resultant precipitate was collected by filtration and dried in vacuo. The solid was purified by preparative HPLC (Method A) to afford 1-(8-((2-(2,6-dioxopiperidin-3-yl)-1,3-dioxoisoindolin-4-yl)amino)octyl)-N-(2-((R)-1-methylpyrrolidin-2-yl)-1H-pyrrolo[3,2-c]pyridin-6-yl)-1H-indazole-5-carboxamide (Example 36, 0.056 g, 11%) as a yellow solid. ¹H NMR (400 MHz, DMSO-d₆) δ ppm 1.23-1.29 (m, 9H), 1.52-1.54 (m, 2H), 1.78-1.93 (m, 5H), 1.98-2.03 (m, 1H), 2.09-2.15 (m, 1H), 2.17 (s, 3H), 2.26-2.28 (m, 1H), 2.51-2.60 (m, 2H), 2.84-2.87 (m, 2H), 3.12-3.16 (m, 2H), 4.42-4.46 (m, 2H), 5.01-5.06 (m, 1H), 6.39 (s, 1H), 6.49 (t, J=5.79 Hz, 1H), 7.01 (d, J=6.80 Hz, 1H), 7.07 (d, J=8.38 Hz, 1H), 7.54-7.59 (m, 1H), 7.75 (d, J=8.76 Hz, 1H), 8.04-8.07 (m, 1H), 8.20-8.22 (m, 2H), 8.51 (s, 1H), 8.55 (s, 1H), 10.40 (s, 1H), 11.07 (br s, 1H), 11.34 (s, 1H). Mass spec: m/z 744.2 [M+H]⁺.

### Example 37 was Synthesised Following Scheme 37

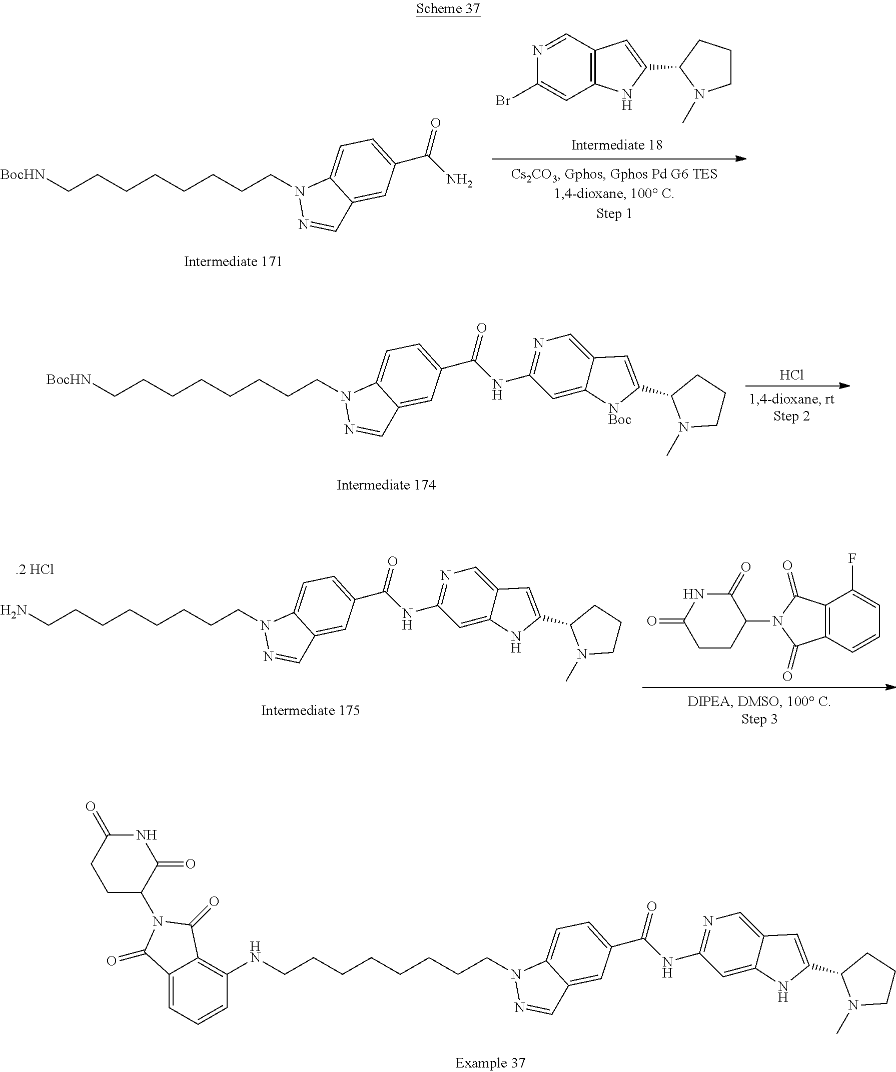

### Step 1

### Intermediate 174: tert-butyl N-{8-[5-({2-[(2S)-1-methylpyrrolidin-2-yl]-1H-pyrrolo[3,2-c]pyridin-6-yl}carbamoyl)indazol-1-yl]octyl}carbamate

A 50 mL round-bottom flask was charged with tert-butyl N-[8-(5-carbamoylindazol-1-yl)octyl]carbamate (Intermediate 171, 210 mg, 0.486 mmol), (2S)-2-{6-bromo-1H-pyrrolo[3,2-c]pyridin-2-yl}-1-methylpyrrolidine (Intermediate 18, 182 mg, 0.583 mmol), Cs₂CO₃ (793 mg, 2.43 mmol), Gphos (94.2 mg, 0.175 mmol), Gphos Pd G6 TES (96.0 mg, 0.102 mmol) and 1,4-dioxane (10 mL) and the reaction was stirred overnight at 100° C. under nitrogen. The volatiles were removed under reduced pressure and the residue was chromatographed on a silica gel column, eluting with MeOH/DCM (4/96), to afford tert-butyl N-{8-[5-({2-[(2S)-1-methylpyrrolidin-2-yl]-1H-pyrrolo[3,2-c]pyridin-6-yl}carbamoyl)indazol-1-yl]octyl}carbamate (Intermediate 174, 140 mg, 47%) as a yellow solid. Mass spec: m/z: 588.40[M+H]⁺.

### Step 2

### Intermediate 175: 1-(8-aminooctyl)-N-{2-[(2S)-1-methylpyrrolidin-2-yl]-1H-pyrrolo[3,2-c]pyridin-6-yl}indazole-5-carboxamide dihydrochloride

Intermediate 175 was synthesised in an analogous manner to Intermediate 173, shown in Step 5 of Scheme 36, using tert-butyl N-{8-[5-({2-[(2S)-1-methylpyrrolidin-2-yl]-1H-pyrrolo[3,2-c]pyridin-6-yl}carbamoyl)indazol-1-yl]octyl}carbamate as the amide, to afford 1-(8-aminooctyl)-N-{2-[(2S)-1-methylpyrrolidin-2-yl]-1H-pyrrolo[3,2-c]pyridin-6-yl}indazole-5-carboxamide dihydrochloride (Intermediate 175). Mass spec: m/z: 488.40 [M+H]⁺.

### Step 3

### Example 37: 1-(8-((2-(2,6-dioxopiperidin-3-yl)-1,3-dioxoisoindolin-4-yl)amino)octyl)-N-(2-((S)-1-methylpyrrolidin-2-yl)-1H-pyrrolo[3,2-c]pyridin-6-yl)-1H-indazole-5-carboxamide

Example 37 was synthesised in an analogous manner to Example 36, shown in Step 6 of Scheme 36, using 1-(8-aminooctyl)-N-{2-[(2S)-1-methylpyrrolidin-2-yl]-1H-pyrrolo[3,2-c]pyridin-6-yl}indazole-5-carboxamide dihydrochloride (Intermediate 175) as the amine and 2-(2,6-dioxopiperidin-3-yl)-4-fluoroisoindoline-1,3-dione (CAS No: 835616-60-9) as the aryl fluoride, to afford 1-(8-((2-(2,6-dioxopiperidin-3-yl)-1,3-dioxoisoindolin-4-yl)amino)octyl)-N-(2-((S)-1-methylpyrrolidin-2-yl)-1H-pyrrolo[3,2-c]pyridin-6-yl)-1H-indazole-5-carboxamide (Example 37). ¹H NMR (400 MHz, MeOH-d4) δ ppm 8.51-8.60 (m, 2H), 8.18-8.23 (m, 2H), 8.06 (d, J=8.8 Hz, 1H), 7.72 (d, J=8.8 Hz, 1H), 7.53 (t, J=7.6 Hz, 1H), 7.01 (d, J=8.4 Hz, 2H), 6.62 (s, 1H), 5.05-5.09 (m, 1H), 4.47-4.51 (m, 2H), 3.70-3.80 (m, 1H), 3.45-3.49 (m, 1H), 3.33-3.37 (m, 1H), 2.82-2.86 (m, 1H), 2.72-2.77 (m, 2H), 2.61-2.63 (m, 1H), 2.40-2.44 (m, 3H), 2.36-2.37 (m, 1H), 2.11-2.18 (m, 3H), 1.94-2.03 (m, 3H), 1.59-1.61 (m, 2H), 1.20-1.21 (m, 9H). Mass spec: m/z: 744.60 [M+H]⁺.

### Example 38 was Synthesised Following Scheme 38

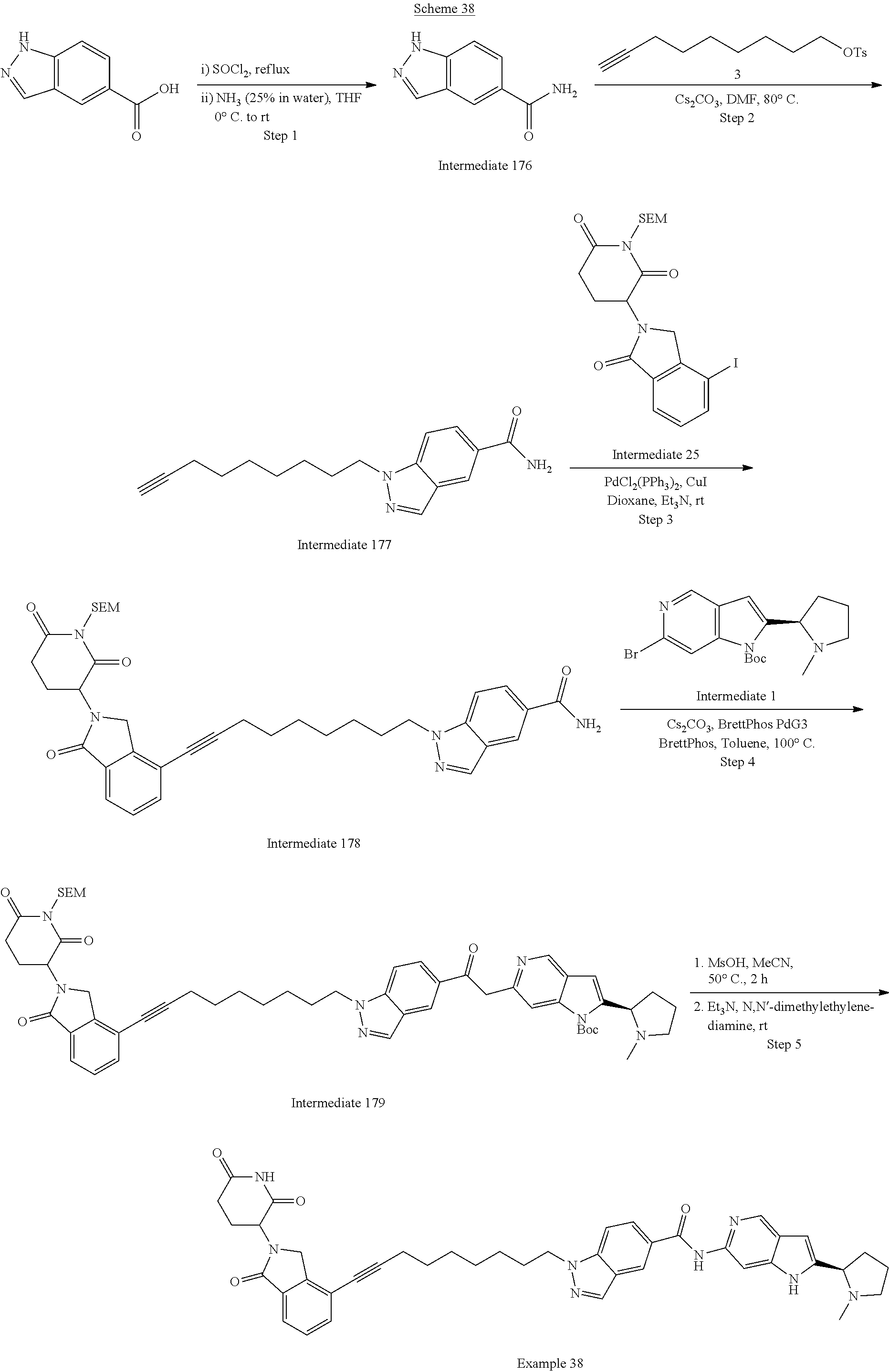

### Step 1

### Intermediate 176: 1H-indazole-5-carboxamide

To solution of 1H-indazole-5-carboxylic acid (CAS No: 61700-61-6, 3.0 g, 18.50 mmol) in thionyl chloride (30 mL) was stirred at reflux for 1 h. The reaction mixture was cooled to room temperature and the solution was concentrated in vacuo. The residue was suspended in dry tetrahydrofuran (80 mL), cooled to 0° C., and 25% NH₃ solution (40 mL, 1100 mmol) was added. The reaction mixture was stirred at room temperature for 6 h. The reaction mixture was diluted with water (100 mL) and the resulting precipitate was collected by filtration and dried to afford 1H-indazole-5-carboxamide (Intermediate 176, 1.5 g, 50%) as a yellow solid. ¹H NMR (400 MHz, DMSO-d₆) δ ppm 7.24 (br s, 1H), 7.55 (d, J=8.80 Hz, 1H), 7.87 (d, J=8.80 Hz, 1H), 7.95 (br s, 1H), 8.18 (s, 1H), 8.35 (s, 1H), 13.23 (br s, 1H). Mass spec: m/z 162 [M+H]⁺.

### Step 2

### Intermediate 177: 1-(non-8-yn-1-yl)-1H-indazole-5-carboxamide

To a solution of 1H-indazole-5-carboxamide (Intermediate 176, 1.80 g, 11.2 mmol) in dimethylformamide (30 mL) was added cesium carbonate (10.9 g, 33.5 mmol) followed by non-8-ynyl-4-methylbenzenesulfonate (CAS No: 87462-64-4, 3.95 g, 13.4 mmol) at room temperature and the reaction mixture was stirred at 80° C. for 4 h. The reaction mixture was then diluted with water (100 mL) and extracted with ethyl acetate (3×60 mL). The combined organic layers were dried over anhydrous Na₂SO₄, filtered and concentrated in vacuo to obtain the crude compound as a mixture of regioisomers. The regioisomers were separated by combi-flash chromatography, by eluting with 60% ethyl acetate in heptane, to afford desired regioisomer 1-(non-8-yn-1-yl)-1H-indazole-5-carboxamide (Intermediate 177, 1.5 g, 47%) as a white solid. ¹H NMR (400 MHz, DMSO-d₆) δ ppm 1.20-1.38 (m, 8H), 1.80-1.84 (m, 2H), 2.08-2.12 (m, 2H), 2.70 (s, 1H), 4.41 (t, J=6.80 Hz, 2H), 7.26 (br s, 1H), 7.69 (d, J=8.60 Hz, 1H), 7.90 (d, J=8.60 Hz, 1H), 7.96 (br s, 1H), 8.17 (s, 1H), 8.33 (s, 1H). Mass spec: m/z: 284.3 [M+H]⁺.

### Step 3

### Intermediate 178: 1-(9-(2-(2,6-dioxo-1-((2-(trimethylsilyl)ethoxy)methyl)piperidin-3-yl)-1-oxoisoindolin-4-yl)non-8-yn-1-yl)-1H-indazole-5-carboxamide

To a solution of 1-(non-8-yn-1-yl)-1H-indazole-5-carboxamide (Intermediate 177, 0.80 g, 2.82 mmol) and 3-(4-iodo-1-oxoisoindolin-2-yl)-1-((2-(trimethylsilyl)ethoxy)methyl)piperidine-2,6-dione (Intermediate 25, 1.55 g, 3.10 mmol) in 1,4-dioxane (6.0 mL) was added triethylamine (13.8 mL, 98.80 mmol). The reaction mixture was purged with argon gas for 15 min then PdCl₂(PPh₃)₂ (0.20 g, 0.28 mmol) and copper (I) iodide (0.05 g, 0.28 mmol) were added and the reaction mixture was further purged with argon gas for 10 min, then stirred at room temperature for 2 h. The reaction mixture was diluted with water (60 mL) and extracted with ethyl acetate (3×50 mL). The combined organic layers were washed with brine solution (2×50 mL), dried over anhydrous Na₂SO₄, filtered and concentrated in vacuo. The crude material was purified by combi-flash chromatography, by eluting with 2% MeOH in dichloromethane, to afford 1-(9-(2-(2,6-dioxo-1-((2-(trimethylsilyl) ethoxy)methyl)piperidin-3-yl)-1-oxoisoindolin-4-yl)non-8-yn-1-yl)-1H-indazole-5-carboxamide (Intermediate 178, 0.80 g, 43%) as a white solid. ¹H NMR (400 MHz, DMSO-d₆) δ ppm −0.04 (s, 9H), 0.76-0.88 (m, 2H), 1.28-1.41 (m, 6H), 1.48-1.57 (m, 2H), 1.80-1.87 (m, 2H), 2.04-2.08 (m, 1H), 2.39-2.46 (m, 3H), 2.77-2.82 (m, 1H), 3.02-3.11 (m, 1H), 3.49-3.56 (m, 2H), 4.23-4.27 (m, 1H), 4.38-4.48 (m, 3H), 5.01-5.09 (m, 2H), 5.25-5.30 (m, 1H), 7.26 (br s, 1H), 7.51 (t, J=7.20 Hz, 1H), 7.61 (d, J=7.20 Hz, 1H), 7.68-7.72 (m, 2H), 7.96 (d, J=8.80 Hz, 1H), 7.96 (br s, 1H), 8.16 (s, 1H), 8.33 (s, 1H). Mass spec: m/z: 654.1 [M−H]—.

### Step 4

### Intermediate 179: tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-1H-indazole-5-carboxamido)-2-((R)-1-methylpyrrolidin-2-yl)-1H-pyrrolo[3,2-c]pyridine-1-carboxylate

To a solution of 1-(9-(2-(2,6-dioxo-1-((2-(trimethylsilyl)ethoxy)methyl)piperidin-3-yl)-1-oxoisoindolin-4-yl) non-8-yn-1-yl)-1H-indazole-5-carboxamide (Intermediate 178, 0.60 g, 0.91 mmol) in toluene (10 mL) was added tert-butyl (R)-6-bromo-2-(1-methylpyrrolidin-2-yl)-1H-pyrrolo[3,2-c]pyridine-1-carboxylate (Intermediate 1, 0.41 g, 1.09 mmol) followed by cesium carbonate (0.89 g, 2.74 mmol). The reaction mixture was purged with argon gas for 15 min, then BrettPhos (0.075 g, 0.13 mmol) and BrettPhos Pd G3 (0.12 g, 0.13 mmol) were added. The reaction mixture was purged with argon gas for 15 min and then stirred at 100° C. for 2 h. The reaction mixture was diluted with water (60 mL) and extracted with ethyl acetate (3×50 mL). The combined organic layers were washed with brine solution (2×50 mL), separated, dried over anhydrous Na₂SO₄, filtered, and concentrated in vacuo. The crude material was purified by combi-flash chromatography, by eluting with 2% MeOH in dichloromethane, to afford tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-1H-indazole-5-carboxamido)-2-((R)-1-methylpyrrolidin-2-yl)-1H-pyrrolo[3,2-c]pyridine-1-carboxylate (Intermediate 179, 0.45 g, 51%) as a white solid. ¹H NMR (400 MHz, DMSO-d₆) δ ppm −0.05 (s, 9H), 0.79-0.83 (m, 2H), 1.18-1.38 (m, 8H), 1.48-1.55 (m, 2H), 1.61-1.65 (m, 1H), 1.70 (s, 9H), 1.80-1.85 (m, 2H), 2.01-2.07 (m, 1H), 2.26-2.30 (m, 2H), 2.36 (s, 3H), 2.41-2.46 (m, 3H), 2.76-2.80 (m, 1H), 3.03-3.15 (m, 2H), 3.48-3.54 (m, 2H), 3.91-3.94 (m, 1H), 4.23-4.27 (m, 1H), 4.42-4.48 (m, 3H), 5.00-5.08 (m, 2H), 5.25-5.29 (m, 1H), 6.75 (br s, 1H), 7.49-7.53 (m, 1H), 7.62 (d, J=8.00 Hz, 1H), 7.70-7.76 (m, 2H), 8.05 (d, J=8.00 Hz, 1H), 8.21 (br s, 1H), 8.55-8.62 (m, 2H), 8.95 (br s, 1H), 10.67 (br s, 1H). Mass spec: m/z: 955.2 [M+H]⁺.

### Step 5

### Example 38: 1-(9-(2-(2,6-dioxopiperidin-3-yl)-1-oxoisoindolin-4-yl)non-8-yn-1-yl)-N-(2-((R)-1-methylpyrrolidin-2-yl)-1H-pyrrolo[3,2-c]pyridin-6-yl)-1H-indazole-5-carboxamide

To a solution of tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-1H-indazole-5-carboxamido)-2-((R)-1-methylpyrrolidin-2-yl)-1H-pyrrolo[3,2-c]pyridine-1-carboxylate (Intermediate 179, 0.42 g, 0.44 mmol) in acetonitrile (10 mL) was added methanesulfonic acid (0.43 mL, 6.59 mmol) and the reaction mixture was stirred at 50° C. for 2 h. The reaction mixture was cooled to room temperature, then N,N′-dimethylethylenediamine (0.32 mL, 2.63 mmol) followed by triethylamine (0.92 mL, 6.59 mmol) were added and the reaction stirred at room temperature for 2 h. The reaction mixture was concentrated in vacuo and the resultant solid washed with ether (2×50 mL), then DCM/Pentanes (2×40 mL) and dried in vacuo. The crude compound was then purified by purified by preparative HPLC (Method A) to afford 1-(9-(2-(2,6-dioxopiperidin-3-yl)-1-oxoisoindolin-4-yl)non-8-yn-1-yl)-N-(2-((R)-1-methylpyrrolidin-2-yl)-1H-pyrrolo[3,2-c]pyridin-6-yl)-1H-indazole-5-carboxamide (Example 38, 0.09 g, 28%) as an off white solid. ¹H NMR (400 MHz, CDCl₃-d) δ ppm 1.24-1.60 (m, 9H), 1.87-2.04 (m, 4H), 2.18-2.26 (m, 2H), 2.29 (s, 3H), 2.35-2.49 (m, 4H), 2.82-2.96 (m, 2H), 3.27-3.32 (m, 1H), 3.49-3.58 (m, 1H), 4.33-4.47 (m, 3H), 4.53-4.58 (m, 1H), 5.24-5.28 (m, 1H), 6.44 (s, 1H), 7.38-7.41 (m, 1H), 7.46-7.49 (m, 1H), 7.59-7.61 (m, 1H), 7.78-7.84 (m, 2H), 8.02-8.04 (m, 1H), 8.43 (s, 1H), 8.47 (s, 1H), 8.64 (s, 1H), 9.25 (br s, 1H) 9.31 (br s, 1H). Mass spec: m/z: 725.1 [M+H]⁺.

### Example 39 was Synthesized Following Scheme 39

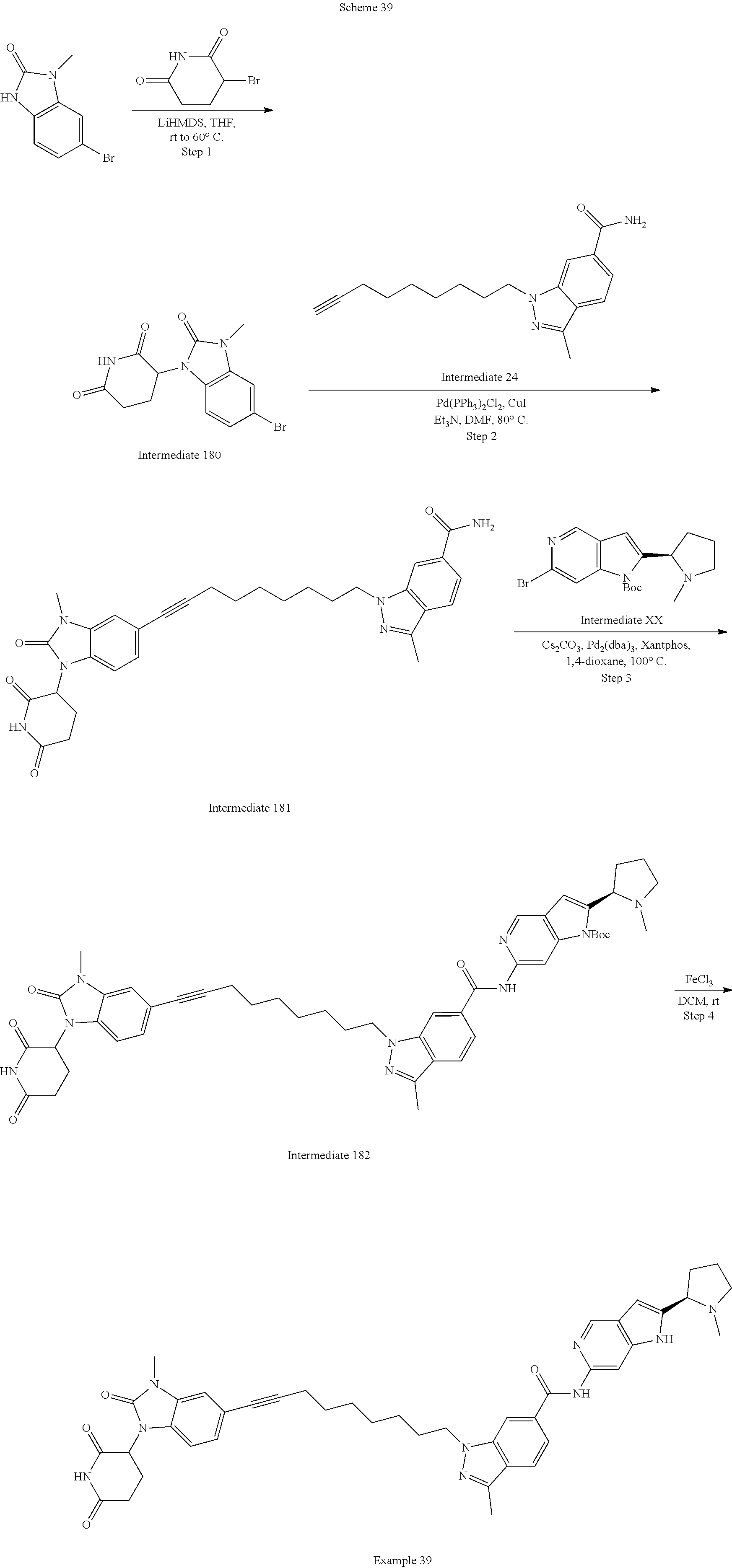

### Step 1

### Intermediate 180: 3-(5-bromo-3-methyl-2-oxo-2,3-dihydro-1H-benzo[d]imidazol-1-yl) piperidine-2,6-dione

To solution of 6-bromo-1-methyl-1,3-dihydro-2H-benzo[d]imidazol-2-one (CAS No. 305790-48-1, 5.00 g, 22.0 mmol) in tetrahydrofuran (30 mL) was added LiHMDS in THF (55 mL, 55.1 mmol) dropwise over a period of 5 min and reaction mixture was stirred at room temperature for 30 min. To the reaction mixture was added to 3-bromopiperidine-2,6-dione (CAS No. 62595-74-8, 8.46 g, 44.0 mmol) in tetrahydrofuran (30 mL) over a period of 5 min, then the reaction was heated at 60° C. for 4 h. The reaction mixture was cooled to room temperature and a cold saturated solution of NH₄Cl (300 mL) was added. The mixture was stirred at room temperature for 20 min. The resultant precipitate was collected by filtration, washed with ethyl acetate (60 mL) and heptane (100 mL) and dried in vacuo to afford 3-(5-bromo-3-methyl-2-oxo-2,3-dihydro-1H-benzo[d]imidazol-1-yl) piperidine-2,6-dione (Intermediate 180, 3.5 g, 47%) as an off white solid which was used for the next step without further purification. ¹H NMR (400 MHz, DMSO-d₆) δ ppm 1.98-2.06 (m, 1H), 2.60-2.75 (m, 2H), 2.82-2.96 (m, 1H), 3.34 (s, 3H), 5.35-5.39 (m, 1H), 7.10 (d, J=8.33 Hz, 1H), 7.21 (d, J=8.11 Hz, 1H), 7.46 (s, 1H). Mass spec m/z 337.7 [M+H]⁺.

### Step 2

### Intermediate 181: 1-(9-(1-(2,6-dioxopiperidin-3-yl)-3-methyl-2-oxo-2,3-dihydro-1H-benzo[d]imidazol-5-yl) non-8-yn-1-yl)-3-methyl-1H-indazole-6-carboxamide

To a mixture of 3-(5-bromo-3-methyl-2-oxo-2,3-dihydro-1H-benzo[d]imidazol-1-yl)piperidine-2,6-dione (Intermediate 180, 1.00 g, 2.95 mmol) and 3-methyl-1-(non-8-yn-1-yl)-1H-indazole-6-carboxamide (Intermediate 24, 1.05 g, 3.54 mmol) in dimethylformamide (10 mL) was added cesium carbonate (3.04 g, 8.87 mmol) and the reaction mixture was purged with argon gas for 10 min then Pd (PPh₃)₂Cl₂ (0.21 g, 0.29 mmol) and copper (I) iodide (0.057 g, 0.29 mmol) were added. The reaction mixture was purged with argon gas for 10 min and the heated at 80° C. for 4 h. The reaction mixture was diluted with water (50 mL) and extracted with ethyl acetate (2×100 mL). The combined organic layers were washed with brine solution (100 mL), dried over anhydrous Na₂SO₄ and concentrated in vacuo. The crude material was purified by combi-flash chromatography, by eluting with 2% MeOH in dichloromethane, to afford 1-(9-(1-(2,6-dioxopiperidin-3-yl)-3-methyl-2-oxo-2,3-dihydro-1H-benzo[d]imidazol-5-yl)non-8-yn-1-yl)-3-methyl-1H-indazole-6-carboxamide (Intermediate 181, 1.00 g, 61%) as a pale yellow solid. ¹H NMR (400 MHz, DMSO-d₆) δ ppm 1.24-1.42 (m, 7H), 1.46-1.48 (m, 2H), 1.79-1.82 (m, 2H), 1.96-2.01 (m, 1H), 2.34-2.37 (m, 1H), 2.57 (s, 3H), 2.82-2.87 (m, 1H), 3.15 (d, J=5.04 Hz, 1H), 3.31 (s, 3H), 3.98-4.08 (m, 1H), 4.31-4.34 (m, 2H), 5.33-5.37 (m, 1H), 7.04 (s, 2H), 7.20 (s, 1H), 7.41 (s, 1H), 7.57 (d, J=8.33 Hz, 1H), 7.71 (d, J=8.33 Hz, 1H), 7.98-8.16 (m, 2H), 11.10 (s, 1H). Mass spec m/z 555.1 [M+H]⁺.

### Step 3

### Intermediate 182: tert-butyl 6-(1-(9-(1-(2,6-dioxopiperidin-3-yl)-3-methyl-2-oxo-2,3-dihydro-1H-benzo[d]imidazol-5-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(9-(1-(2,6-dioxopiperidin-3-yl)-3-methyl-2-oxo-2,3-dihydro-1H-benzo[d]imidazol-5-yl)non-8-yn-1-yl)-3-methyl-1H-indazole-6-carboxamide (Intermediate 181, 0.60 g, 1.08 mmol) and tert-butyl (R)-6-bromo-2-(1-methylpyrrolidin-2-yl)-1H-pyrrolo[3,2-c]pyridine-1-carboxylate (Intermediate 1, 0.49 g, 1.29 mmol) in 1,4-dioxane (14 mL) was added cesium carbonate (1.06 g, 3.24 mmol). The reaction mixture was purged with argon gas for 10 min, then Xantphos (0.19 g, 0.32 mmol) and Pd₂(dba)₃ (0.15 g, 0.16 mmol) were added. The reaction mixture was purged with argon gas for 10 min and then heated at 100° C. for 2 h. The reaction mixture was diluted with water (40 mL) and extracted with ethyl acetate (3×50 mL). The combined organic layers were washed with brine solution (2×50 mL), dried over anhydrous Na₂SO₄, and concentrated in vacuo. The crude material was purified by combi-flash chromatography, by eluting with 4% MeOH in dichloromethane, to afford tert-butyl 6-(1-(9-(1-(2,6-dioxopiperidin-3-yl)-3-methyl-2-oxo-2,3-dihydro-1H-benzo[d]imidazol-5-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 182, 0.43 g, 47%) as a yellow solid. ¹H NMR (400 MHz, DMSO-d₆) δ ppm 1.18-1.40 (m, 8H), 1.48-1.53 (m, 2H), 1.60-1.66 (m, 2H), 1.70 (s, 9H), 1.73-1.80 (m, 2H), 1.86-1.90 (m, 2H), 1.98-2.03 (m, 1H), 2.14-2.20 (m, 1H), 2.36 (s, 3H), 2.37-2.42 (m, 5H), 2.60-2.74 (m, 2H), 2.84-2.94 (m, 1H), 3.11-3.18 (m, 2H), 3.91-3.94 (m, 1H), 4.38-4.42 (m, 2H), 5.34-5.40 (m, 1H), 6.76 (s, 1H), 7.04-7.06 (m, 2H), 7.20 (s, 1H), 7.71-7.80 (m, 2H), 8.43 (s, 1H), 8.61 (s, 1H), 8.98 (s, 1H), 10.75 (br s, 1H), 11.10 (br s, 1H). Mass spec m/z 854.2 [M+H]⁺.

### Step 4

### Example 39: 1-(9-(1-(2,6-dioxopiperidin-3-yl)-3-methyl-2-oxo-2,3-dihydro-1H-benzo[d]imidazol-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(9-(1-(2,6-dioxopiperidin-3-yl)-3-methyl-2-oxo-2,3-dihydro-1H-benzo[d]imidazol-5-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 182, 0.13 g, 0.15 mmol) in dichloromethane (8 mL) was added ferric chloride (0.074 g, 0.45 mmol) and the reaction was stirred at room temperature for 48 h. The reaction mixture was concentrated in vacuo, the resultant precipitate collected by filtration, washed with pentane (2×20 mL) and dried in vacuo to obtain the crude compound. The crude material was purified by preparative HPLC (Method A) to afford 1-(9-(1-(2,6-dioxopiperidin-3-yl)-3-methyl-2-oxo-2,3-dihydro-1H-benzo[d]imidazol-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 39, 0.025 g, 22%) as an off white solid. ¹H NMR (400 MHz, DMSO-d₆) δ ppm 1.31-1.40 (m, 7H), 1.48-1.56 (m, 2H), 1.78-2.05 (m, 7H), 2.13-2.15 (m, 2H), 2.17 (s, 3H), 2.24-2.30 (m, 1H), 2.37-2.40 (m, 2H), 2.51 (s, 3H), 2.59-2.70 (m, 2H), 2.84-2.94 (m, 1H), 3.12-3.16 (m, 1H), 3.34-3.36 (m, 1H), 4.41 (t, J=6.88 Hz, 2H), 5.34-5.38 (m, 1H), 6.40 (s, 1H), 7.05-7.08 (m, 2H), 7.20 (s, 1H), 7.71-7.79 (m, 2H), 8.25 (s, 1H), 8.44 (s, 1H), 8.51 (s, 1H), 10.52 (s, 1H), 11.10 (s, 1H), 11.38 (s, 1H). Mass spec m/z 754.3 [M+H]⁺.

### Example 40 was Synthesized Following Scheme 40

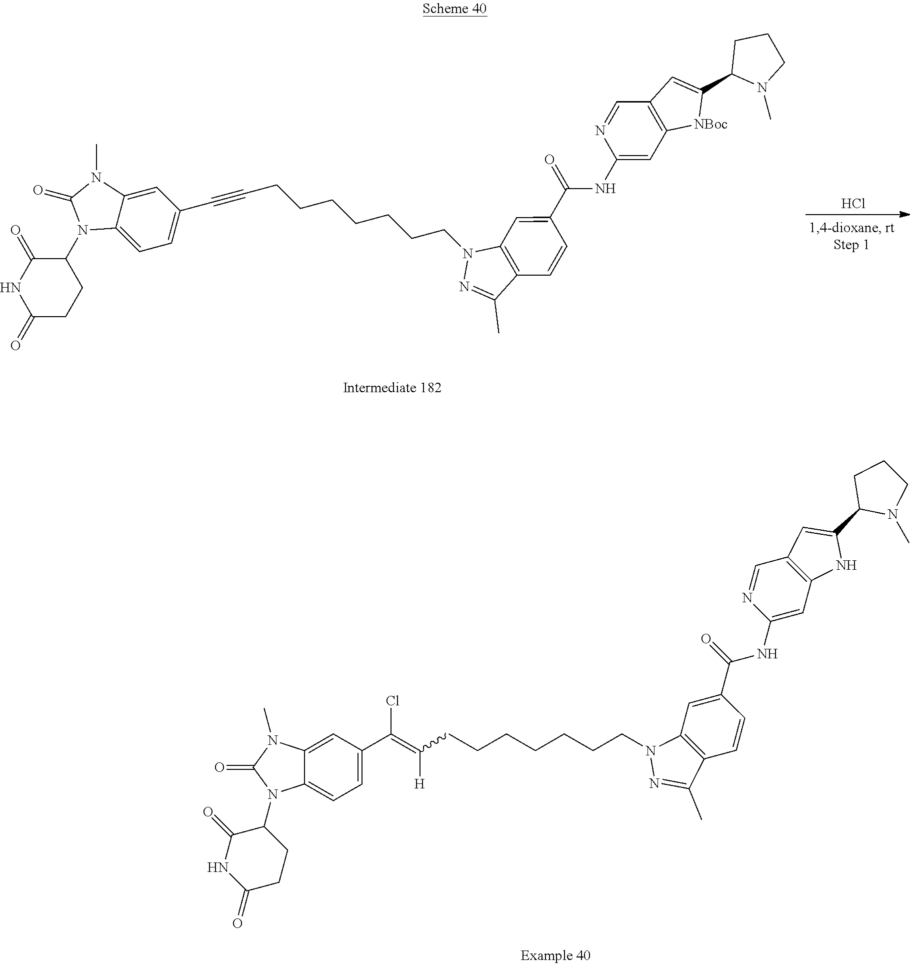 [SMILES: Cc1nn(CCCCCCCC#Cc2ccc3c(c2)n(C)c(=O)n3C2CCC(=O)NC2=O)c2cc(C(=O)Nc3cc4c(cn3)cc([C@H]3CCCN3C)n4C(=O)OC(C)(C)C)ccc12 ; Cl ; Cc1nn(CCCCCCCC=C(Cl)c2ccc3c(c2)n(C)c(=O)n3C2CCC(=O)NC2=O)c2cc(C(=O)Nc3cc4[nH]c([C@H]5CCCN5C)cc4cn3)ccc12]

### Step 1

### Example 40: 1-((E)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide and 1-((Z)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(9-(1-(2,6-dioxopiperidin-3-yl)-3-methyl-2-oxo-2,3-dihydro-1H-benzo[d]imidazol-5-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 182, 0.20 g, 0.23 mmol) in 1,4-dioxane (8.0 mL) was added 4M HCl in 1,4-dioxane (5.0 mL) and the mixture was stirred at room temperature for 4 h then concentrated in vacuo. The resultant solid was washed with dichloromethane (2×20 mL) and dried in vacuo to obtain the crude compound which was purified by preparative HPLC (Method A) to afford as a 40:60 ratio of E and Z-alkene isomers of 1-((E)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide and 1-((Z)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide (Example 40, 0.045 g, 25%) as an off white solid. ¹H NMR (400 MHz, DMSO-d₆) δ ppm 1.17-1.48 (m, 8H), 1.76-1.95 (m, 5H), 1.99-2.09 (m, 2H), 2.11-2.15 (m, 1H), 2.17 (s, 3H), 2.23-2.34 (m, 2H), 2.52 (s, 3H), 2.57-2.75 (m, 2H), 2.82-2.94 (m, 1H), 3.11-3.18 (m, 1H), 3.32-3.33 (m, 1H), 3.34 (s, 3H), 4.34-4.43 (m, 2H), 5.35-5.40 (m, 1H), 5.95 (t, J=7.4 Hz, 0.6H), 6.31 (t, J=7.4 Hz, 0.4H), 6.40 (s, 1H), 7.00-7.39 (m, 3H), 7.71-7.80 (m, 2H), 8.25 (s, 1H), 8.41-8.44 (m, 1H), 8.52 (s, 1H), 10.52 (s, 1H), 11.10 (br s, 1H), 11.38 (s, 1H). Mass spec m/z 790.1 [M+H]⁺.

### Example 41 was Synthesized Following Scheme 41

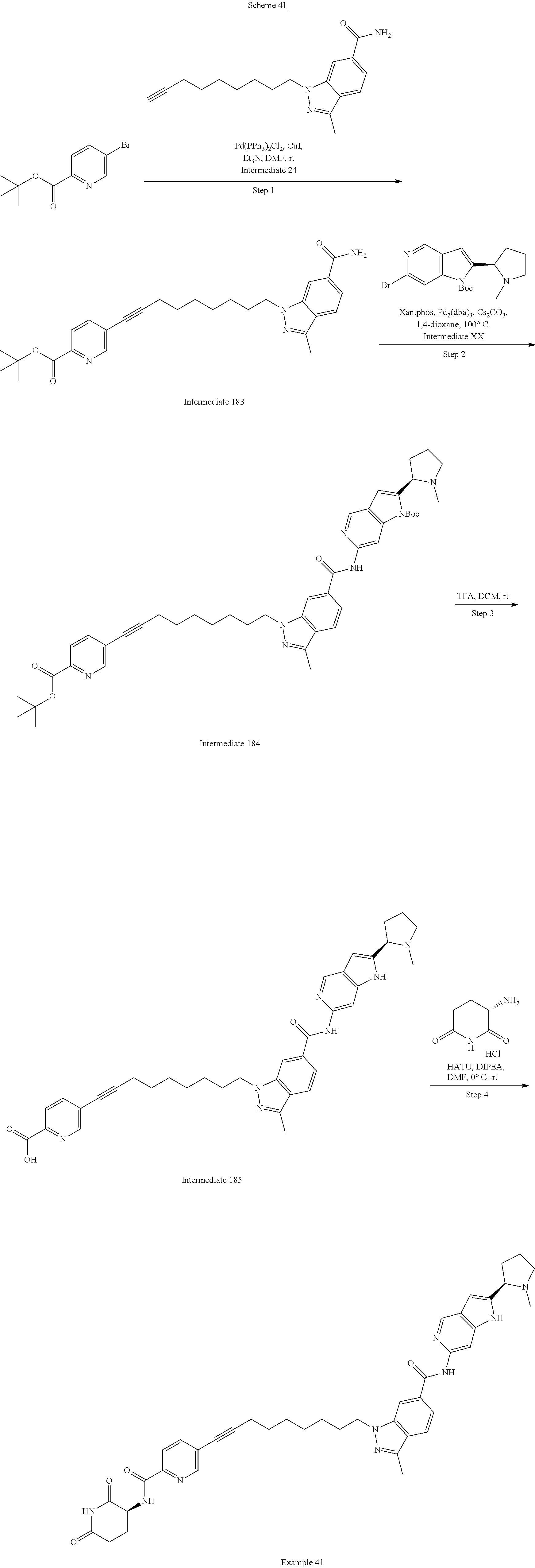

### Step 1

### Intermediate 183: tert-butyl 5-(9-(6-carbamoyl-3-methyl-1H-indazol-1-yl)non-1-yn-1-yl)picolinate

A solution of tert-butyl-5-bromopicolinate (CAS No: 845306-08-3, 1.5 g, 5.81 mmol), 3-methyl-1-(non-8-yn-1-yl)-1H-indazole-6-carboxamide (Intermediate 24, 1.73 g, 5.81 mmol) and triethylamine (21 g, 209 mmol) in dimethylformamide (10 mL) was degassed with argon gas for 10 min, then copper (I) iodide (0.11 g, 0.58 mmol) and Pd(PPh₃)₂Cl₂ (0.42 g, 0.58 mmol) were added. The resulting reaction mixture was stirred at room temperature for 3 h then poured into water (60 mL), extracted with ethyl acetate (3×60 mL). The combined organic layers were dried over anhydrous Na₂SO₄, filtered, and concentrated in vacuo The crude compound was purified by combi-flash column chromatography, by eluting with 80% ethyl acetate in heptane, to afford tert-butyl 5-(9-(6-carbamoyl-3-methyl-1H-indazol-1-yl) non-1-yn-1-yl) picolinate (Intermediate 183, 1.60 g, 58%) as an off-white solid. ¹H NMR (400 MHz, DMSO-d₆) δ ppm 1.24-1.34 (m, 4H), 1.49-1.51 (m, 4H), 1.51 (s, 9H), 1.80-1.82 (m, 2H), 2.42-2.44 (m, 2H), 2.47 (s, 3H), 4.30-4.44 (m, 2H), 7.39-7.40 (m, 1H), 7.56-7.58 (m, 2H), 7.70 (d, J=8.40 Hz, 1H), 7.91 (br s, 2H), 8.10 (s, 1H), 8.63 (s, 1H). Mass spec: m/z 475.1 [M+H]⁺.

### Step 2

### Intermediate 184: tert-butyl-(R)-6-(1-(9-(6-(tert-butoxycarbonyl)pyridin-3-yl)non-8-yn-1-yl)-3-methyl-1H-indazole-6-carboxamido)-2-(1-methylpyrrolidin-2-yl)-1H-pyrrolo[3,2-c]pyridine-1-carboxylate

To a solution of tert-butyl 5-(9-(6-carbamoyl-3-methyl-1H-indazol-1-yl) non-1-yn-1-yl) picolinate (Intermediate 183, 800 mg, 1.69 mmol), in 1,4-dioxane (20 mL) was added tert-butyl-(R)-6-bromo-2-(1-methylpyrrolidin-2-yl)-1H-pyrrolo[3,2-c]pyridine-1-carboxylate (Intermediate 1, 0.51 g, 1.35 mmol) and cesium carbonate (1.10 g, 3.38 mmol). The reaction mixture was degassed with argon gas for 10 min, then Pd₂(dba)₃ (297 mg, 0.31 mmol) followed by Xantphos (376 mg, 0.51 mmol) were added and reaction mixture was purged again with argon gas for 5 min, then heated at 100° C. for 2 h. The reaction mixture was concentrated in vacuo to obtain crude compound which was purified by combi-flash column chromatography, by eluting with 80% ethyl acetate in heptane, to obtain tert-butyl-(R)-6-(1-(9-(6-(tert-butoxycarbonyl)pyridin-3-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 184, 550 mg, 34%) as an off-white solid. ¹H NMR (500 MHz, DMSO-d₆) δ ppm 1.31-1.37 (m, 8H), 1.46 (s, 9H) 1.47-1.54 (br s, 5H), 1.69 (s, 9H), 1.70-1.80 (m, 3H), 1.90-1.91 (m, 2H), 2.35 (s, 3H), 2.36-2.27 (m, 2H), 3.13-3.14 (m, 1H), 3.92-3.98 (m, 1H), 4.41-4.42 (m, 2H), 6.75 (s, 1H), 7.56-7.62 (m, 2H), 7.70-7.77 (m, 2H), 7.90 (s, 1H), 8.43 (s, 1H), 8.62-8.63 (m, 1H), 8.97 (s, 1H), 10.76 (s, 1H). Mass spec: m/z 774.2 [M+H]⁺.

### Step 3

### Intermediate 185: (R)-5-(9-(3-methyl-6-((2-(1-methylpyrrolidin-2-yl)-1H-pyrrolo[3,2-c]pyridin-6-yl)carbamoyl)-1H-indazol-1-yl)non-1-yn-1-yl)picolinic acid trifluoroacetate

To a cooled (0° C.) solution of tert-butyl-(R)-6-(1-(9-(6-(tert-butoxycarbonyl)pyridin-3-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 184, 0.52 g, 0.567 mmol) in dichloromethane (10 mL) was added TFA (4.0 mL) and the resulting reaction mixture was stirred at room temperature for 16 h. The reaction mixture was concentrated in vacuo to obtain crude (R)-5-(9-(3-methyl-6-((2-(1-methylpyrrolidin-2-yl)-1H-pyrrolo[3,2-c]pyridin-6-yl)carbamoyl)-1H-indazol-1-yl)non-1-yn-1-yl)picolinic acid trifluoroacetate (Intermediate 185, 400 mg) which was used for next step without further purification. ¹H NMR (500 MHz, DMSO-d₆) δ ppm 1.08-1.10 (m, 1H), 1.31-1.37 (m, 6H), 1.52-1.54 (m, 3H), 1.85-1.88 (m, 3H), 2.19 (s, 3H), 2.45-2.54 (m, 4H), 2.87 (s, 3H), 3.30-3.31 (m, 1H), 3.80-3.81 (m, 1H), 4.43-4.44 (m, 1H), 4.70-4.71 (m, 1H), 7.10 (s, 1H), 7.54-7.62 (m, 3H), 7.74 (d, J=8.0 Hz, 1H), 7.87-7.98 (m, 3H), 8.11 (s, 1H), 8.63 (s, 1H), 8.98 (s, 2H), 11.40 (br s, 1H).

### Step 4

### Example 41: 1-(9-(6-(((S)-2,6-dioxopiperidin-3-yl)carbamoyl)pyridin-3-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

To a cooled (0° C.) solution of (R)-5-(9-(3-methyl-6-((2-(1-methylpyrrolidin-2-yl)-1H-pyrrolo[3,2-c]pyridin-6-yl)carbamoyl)-1H-indazol-1-yl)non-1-yn-1-yl)picolinic acid trifluoroacetate (Intermediate 185, 400 mg, 0.64 mmol), (S)-3-aminopiperidine-2,6-dione hydrochloride (CAS No: 25181-50-4, 126 mg, 0.77 mmol) and HATU (388 mg, 0.97 mmol) in dimethylformamide (10.0 mL) was added DIPEA (440 mg, 3.23 mmol). The resulting reaction mixture was stirred at room temperature for 16 h then poured into water (15 mL). The resultant precipitate was collected by filtration and dried in vacuo to obtain the crude compound as brown solid (400 mg) which was purified by preparative HPLC (Method A) to obtain 1-(9-(6-(((S)-2,6-dioxopiperidin-3-yl)carbamoyl)pyridin-3-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 41, 85 mg 18%) as an off-white solid. ¹H NMR (400 MHz, DMSO-d₆) δ ppm 1.30-1.40 (m, 6H), 1.54-1.57 (m, 2H), 1.84-1.88 (m, 5H), 1.90-1.95 (m, 1H), 2.17 (s, 3H) 2.18-2.25 (m, 3H), 2.45-2.47 (m, 3H), 2.49 (s, 3H), 2.52-2.54 (m, 1H), 3.13-3.17 (m, 1H), 3.30-3.31 (m, 1H), 4.41 (t, J=7.2 Hz, 2H), 4.72-4.79 (m, 1H), 6.40 (s, 1H), 7.72-7.79 (m, 2H), 7.94-8.00 (m, 2H), 8.24 (s, 1H), 8.44 (s, 1H), 8.52 (s, 1H), 8.63-8.64 (m, 1H), 9.06 (d, J=8.4 Hz, 1H), 10.51 (s, 1H), 10.91 (s, 1H), 11.41 (s, 1H). Mass spec: m/z 728.1 [M+H]⁺.

### Example 42 was Synthesized Following Scheme 42

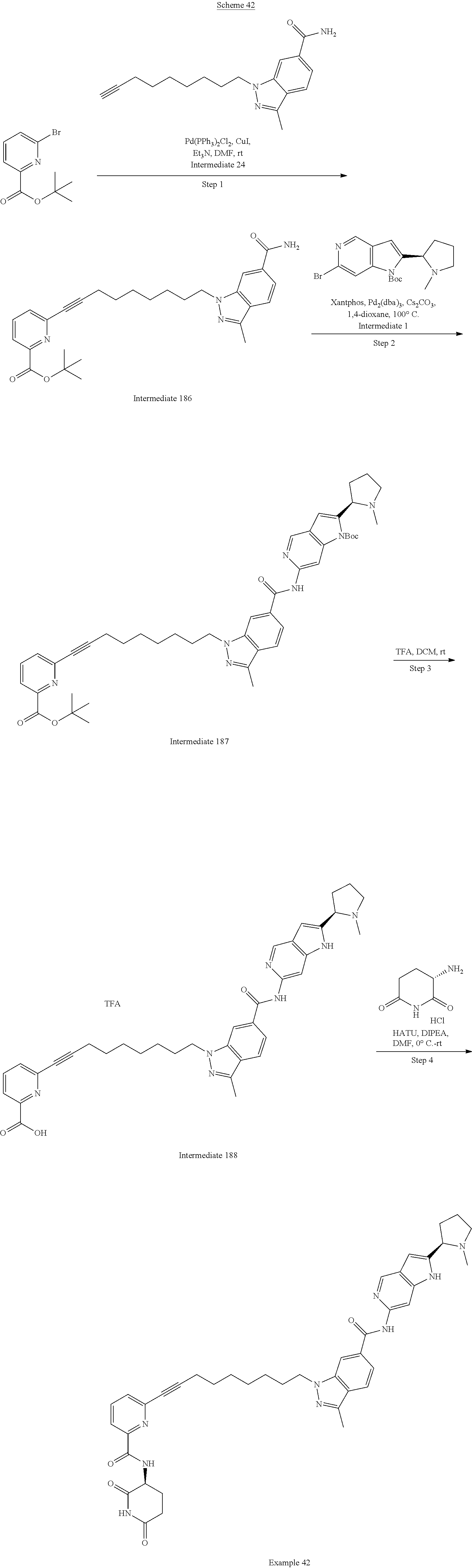

### Step 1

### Intermediate 186: tert-butyl 6-(9-(6-carbamoyl-3-methyl-1H-indazol-1-yl) non-1-yn-1-yl) picolinate

To a solution of tert-butyl 6-bromopicolinate (CAS No: 910044-07-4, 1.5 g, 5.81 mmol) in dimethylformamide (10 mL) was added 3-methyl-1-non-8-ynyl-indazole-6-carboxamide (Intermediate 24, 1.7 g, 5.81 mmol) and triethylamine (29 g, 209 mmol). The resulting solution was degassed with argon gas for 10 min, then copper (I) iodide (0.11 g, 0.58 mmol) and PdCl₂(PPh₃)₂ (0.42 g, 0.58 mmol) were added and the reaction mixture was again purged with argon gas for 10 min. The resulting reaction mixture was stirred at room temperature for 3 h. The reaction mixture was poured into water (60 mL) and extracted with ethyl acetate (3×60 mL). The combined organic layers were dried over anhydrous Na₂SO₄, filtered, and concentrated in vacuo. The crude material was purified by combi-flash column chromatography, by eluting with 80% ethyl acetate in heptane, to afford tert-butyl 6-(9-(6-carbamoyl-3-methyl-1H-indazol-1-yl) non-1-yn-1-yl) picolinate (Intermediate 186, 1.90 g, 69%) as an off white solid. ¹H NMR (400 MHz, DMSO-d₆) δ ppm 1.15-1.19 (m, 2H), 1.27-1.35 (m, 6H), 1.50-1.51 (m, 2H) 1.52 (s, 9H), 1.82-1.84 (m, 2H), 1.44 (s, 3H), 4.33-4.35 (m, 2H), 7.42 (s, 1H), 7.58-7.60 (m, 2H), 7.72 (d, J=7.6 Hz, 1H), 7.90 (br s, 2H), 8.04 (br s, 1H), 8.13 (br s, 1H). Mass spec m/z 475.10 [M+H]⁺.

### Step 2

### Intermediate 187: tert-butyl (R)-6-(1-(9-(6-(tert-butoxycarbonyl)pyridin-2-yl)non-8-yn-1-yl)-3-methyl-1H-indazole-6-carboxamido)-2-(1-methylpyrrolidin-2-yl)-1H-pyrrolo[3,2-c]pyridine-1-carboxylate

To a solution of tert-butyl 6-(9-(6-carbamoyl-3-methyl-1H-indazol-1-yl)non-1-yn-1-yl)picolinate (Intermediate 186, 0.798 g, 1.68 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 1, 0.80 g, 2.10 mmol) and cesium carbonate (1.38 g, 4.20 mmol) and the solution was purged with argon gas for 10 min prior to the addition of Pd₂(dba)₃ (297 mg, 0.31 mmol) followed by Xantphos (376 mg, 0.63 mmol). The resulting reaction mixture was heated at 100° C. for 2 h. The mixture was concentrated in vacuo to obtain crude compound which was purified by combi-flash chromatography, by eluting with 80% ethyl acetate in heptane, to afford tert-butyl (R)-6-(1-(9-(6-(tert-butoxycarbonyl)pyridin-2-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 187, 510 mg, 31%) as a yellow solid. ¹H NMR (500 MHz, DMSO-d6) δ ppm 1.3-1.40 (m, 8H), 1.5 (s, 9H), 1.56-159 (m, 4H), 1.71 (s, 9H), 1.75-1.77 (m, 1H), 1.86-1.91 (m, 2H), 2.33 (s, 3H), 2.43-2.46 (m, 2H), 2.52 (s, 3H), 3.12-3.15 (m, 1H), 3.91-3.94 (m, 1H), 4.34-4.43 (m, 2H), 6.76 (s, 1H), 7.59-7.60 (m, 1H), 7.71-7.74 (m, 2H), 7.88-7.90 (m, 2H), 8.44 (s, 1H), 8.60 (d, J=8 Hz, 1H), 8.98 (s, 1H), 10.77 (s, 1H). Mass spec m/z 774.4 [M+H]⁺.

### Step 3

### Intermediate 188: (R)-6-(9-(3-methyl-6-((2-(1-methylpyrrolidin-2-yl)-1H-pyrrolo[3,2-c]pyridin-6-yl)carbamoyl)-1H-indazol-1-yl)non-1-yn-1-yl)picolinic acid trifluoroacetate

To a cooled (0° C. solution of tert-butyl (R)-6-(1-(9-(6-(tert-butoxycarbonyl)pyridin-2-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 187, 500 mg, 0.65 mmol) in DCM (5 mL) was added TFA (2 mL). The resulting reaction mixture was stirred at room temperature for 16 h. The reaction mixture was concentrated in vacuo to obtain crude (R)-6-(9-(3-methyl-6-((2-(1-methylpyrrolidin-2-yl)-1H-pyrrolo[3,2-c]pyridin-6-yl)carbamoyl)-1H-indazol-1-yl)non-1-yn-1-yl)picolinic acid trifluoroacetate (Intermediate 88, 390 mg) as a brown sticky solid which was used directly in next step without further purification. Mass spec m/z 618.42 [M+H]⁺.

### Step 4

### Example 42: 1-(9-(6-(((S)-2,6-dioxopiperidin-3-yl)carbamoyl)pyridin-2-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

To a cooled (0° C.) solution of (R)-6-(9-(3-methyl-6-((2-(1-methylpyrrolidin-2-yl)-1H-pyrrolo[3,2-c]pyridin-6-yl)carbamoyl)-1H-indazol-1-yl)non-1-yn-1-yl)picolinic acid trifluoroacetate (Intermediate 188, 400 mg, 0.64 mmol), (S)-3-aminopiperidine-2,6-dione hydrochloride (CAS No: 25181-50-4, 127 mg, 0.77 mmol) and HATU (388 mg, 0.97 mmol) in dimethylformamide (10 mL) was added DIPEA (440 mg, 3.23 mmol). The resulting reaction mixture was stirred at room temperature for 16 h. The mixture was poured into water (15 mL), the resultant brown precipitate collected by filtration and dried in vacuo. The crude solid was purified by preparative HPLC (Method A) to afford 1-(9-(6-(((S)-2,6-dioxopiperidin-3-yl)carbamoyl)pyridin-2-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 42, 75 mg, 16%) as an off white solid. ¹H NMR (400 MHz, DMSO-d₆) δ ppm 1.30-1.38 (m, 7H), 1.53-1.57 (m, 2H), 1.85-1.97 (m, 7H), 1.98-2.18 (m, 3H), 2.20 (s, 3H), 2.49 (s, 3H), 2.54-2.55 (m, 2H), 2.67-2.75 (m, 1H), 3.20-3.31 (m, 1H), 4.40 (t, J=7.20 Hz, 2H), 4.77-4.81 (m, 1H), 6.40 (s, 1H), 7.62-7.64 (m, 1H), 7.77-7.80 (m, 2H), 7.92-8.00 (m, 2H), 8.25 (s, 1H), 18.43 (s, 1H), 8.52 (s, 1H), 8.96 (d, J=8.80 Hz, 1H), 10.56 (s, 1H), 11.50 (s, 1H). Mass spec m/z 728.10 [M+H]⁺.

### Example 43 was Synthesized Following Scheme 43

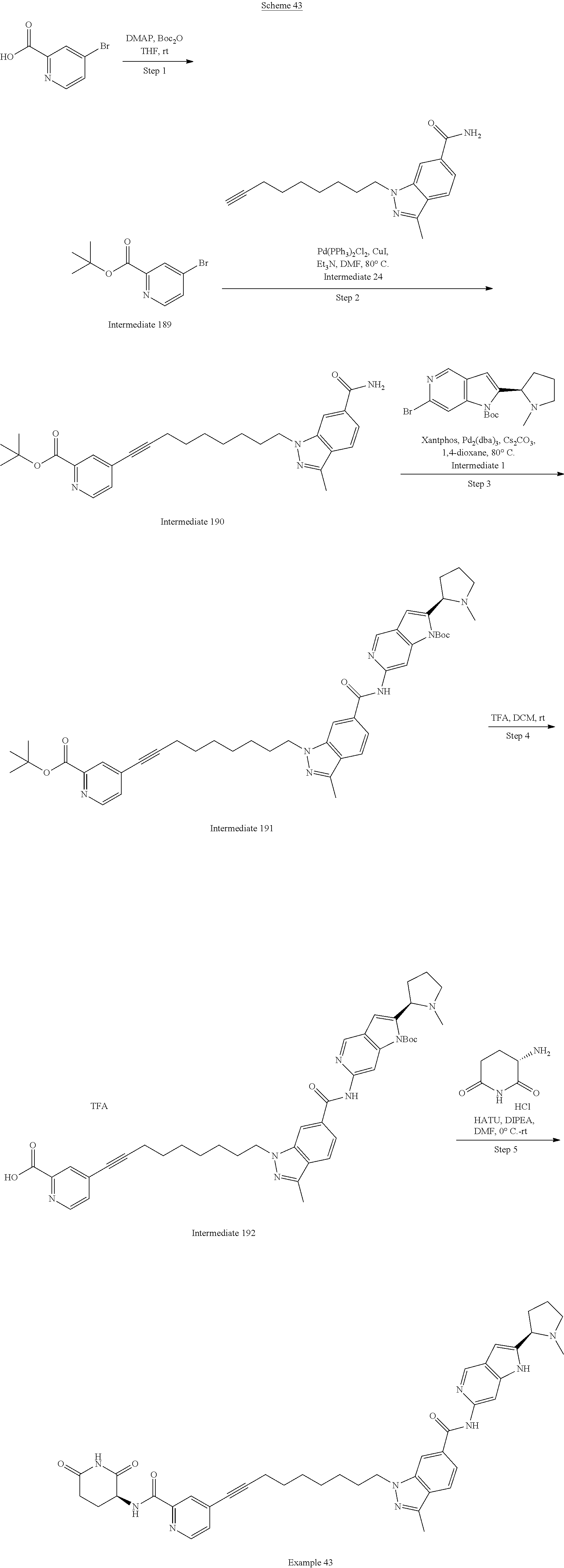

### Step 1

### Intermediate 189: tert-butyl 4-bromopicolinate

To a solution of 4-bromopyridine-2-carboxylic acid (CAS No: 30766-03-1, 5.50 g, 27 mmol) and 4-dimethylaminopyridine (1.66 g, 14 mmol) in tetrahydrofuran (120 mL) was added di-tert-butyldicarbonate (8.80 g, 41 mmol) dropwise over 10 min and the reaction mixture was stirred at room temperature for 16h. Water (100 mL) was added and the mixture extracted with diethyl ether (2×100 mL). The combined organic layers were washed sequentially with water (100 mL), 5% NaHCO₃ (50 mL) and brine solution (50 mL). The combined organic layers were dried over anhydrous Na₂SO₄ and concentrated in vacuo to afford the crude product which was purified by combi-flash column chromatography, by eluting with 30% ethyl acetate in heptane, to afford tert-butyl 4-bromopicolinate (Intermediate 189, 6 g, 85%) as a colourless liquid. ¹H NMR (400 MHz, DMSO-d₆) δ ppm 1.55 (s, 9H), 7.90 (d, J=4.4 Hz, 1H), 8.13 (s, 1H), 8.57 (d, J=4.8 Hz, 1H). Mass spec m/z 259.90 [M+H]⁺.

### Step 2

### Intermediate 190: tert-butyl 4-(9-(6-carbamoyl-3-methyl-1H-indazol-1-yl)non-1-yn-1-yl)picolinate

To a solution of 3-methyl-1-(non-8-yn-1-yl)-1H-indazole-6-carboxamide (Intermediate 24, 1.65 g, 5.54 mmol) in acetonitrile (20 mL) was added tert-butyl 4-bromopyridine-2-carboxylate (Intermediate 189, 1.30 g, 5.04 mmol) followed by triethylamine (2.05 g, 20.16 mmol). The reaction mixture was purged with argon gas for 10 min, then PdCl₂(PPh₃)₂ (353 mg, 0.504 mmol) and copper (I) iodide (96 mg, 0.504 mmol) were added and the reaction was purged with argon gas for 10 min then stirred at 80° C. for 2 h. The reaction mixture was concentrated in vacuo and the crude residue was diluted with saturated sodium thiosulphate solution (200 mL) and extracted with ethyl acetate (3×200 mL). The combined organic layers were dried over anhydrous Na₂SO₄ and concentrated in vacuo. The crude product was purified by combi-flash column chromatography, by eluting with 80% ethyl acetate in heptane, to afford tert-butyl 4-(9-(6-carbamoyl-3-methyl-1H-indazol-1-yl) non-1-yn-1-yl) picolinate (Intermediate 190, 1.8 g, 75%) as a yellow solid. ¹H NMR (500 MHz, DMSO-d₆) δ ppm 1.26-1.35 (m, 8H), 1.54 (s, 9H), 1.83-1.85 (m, 2H), 2.44-2.46 (m, 2H), 2.49 (s, 3H), 4.33-4.34 (m, 2H), 7.45 (br s, 1H), 7.54-7.60 (m, 2H), 7.71-7.77 (m, 2H), 7.84 (s, 1H), 8.10 (br s, 1H), 8.12 (s, 1H). Mass spec m/z 475.1 [M+H]⁺.

### Step 3

### Intermediate 191: tert-butyl (R)-6-(1-(9-(2-(tert-butoxycarbonyl)pyridin-4-yl)non-8-yn-1-yl)-3-methyl-1H-indazole-6-carboxamido)-2-(1-methylpyrrolidin-2-yl)-1H-pyrrolo[3,2-c]pyridine-1-carboxylate

To a solution of tert-butyl (R)-6-bromo-2-(1-methylpyrrolidin-2-yl)-1H-pyrrolo[3,2-c]pyridine-1-carboxylate (Intermediate 1, 1.0 g, 2.70 mmol) in 1,4-dioxane (15 mL) was added tert-butyl 4-(9-(6-carbamoyl-3-methyl-1H-indazol-1-yl)non-1-yn-1-yl)picolinate (Intermediate 190, 0.9 g, 1.89 mmol) and cesium carbonate (1.86 g, 5.70 mmol) and the solution was degassed with argon gas for 10 min. Pd₂(dba)₃ (260 mg, 0.31 mmol) was added, followed by Xantphos (328 mg, 0.57 mmol) and the mixture was purged again with argon gas for 10 min then heated at 80° C. for 2 h. The reaction mixture was concentrated in vacuo and the crude material was purified by combi-flash chromatography, by eluting with 80% ethyl acetate in heptane, to obtain tert-butyl (R)-6-(1-(9-(2-(tert-butoxycarbonyl)pyridin-4-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 191, 0.81 g, 50%) as a yellow solid. ¹H NMR (400 MHz, DMSO-d₆) δ ppm 1.17-1.90 (m, 1H), 1.29-1.36 (m, 6H), 1.53 (s, 9H) 1.54-1.65 (m, 3H), 1.68-1.74 (m, 3H), 1.75 (s, 9H), 1.87-1.89 (m, 2H), 2.35 (s, 3H), 2.40-2.50 (m, 2H), 2.51 (s, 3H), 3.12-3.13 (m, 1H), 3.92-3.93 (m, 1H), 4.39-4.40 (m, 2H), 6.75 (s, 1H), 7.50-7.52 (m, 1H), 7.71-7.81 (m, 3H), 8.43 (s, 1H), 8.61-8.62 (m, 2H), 8.97 (s, 1H), 10.75 (s, 1H). Mass spec m/z 774.3 [M+H]⁺.

### Step 4

### Intermediate 192: (R)-4-(9-(3-methyl-6-((2-(1-methylpyrrolidin-2-yl)-1H-pyrrolo[3,2-c]pyridin-6-yl) carbamoyl)-1H-indazol-1-yl) non-1-yn-1-yl)picolinic acid trifluoroacetate

To a cooled (0° C.) solution of tert-butyl (R)-6-(1-(9-(2-(tert-butoxycarbonyl)pyridin-4-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 191, 800 mg, 1.03 mmol) in DCM (3 mL) was added TFA (3 mL). The resulting reaction mixture was stirred at room temperature for 16 h then concentrated in vacuo to obtain crude (R)-4-(9-(3-methyl-6-((2-(1-methylpyrrolidin-2-yl)-1H-pyrrolo[3,2-c]pyridin-6-yl)carbamoyl)-1H-indazol-1-yl)non-1-yn-1-yl)picolinic acid trifluoroacetate (Intermediate 192, 0.90 g) as a yellow solid which was used directly into next step without further purification. Mass spec m/z 618.34 [M+H]⁺.

### Step 5

### Example 43: 1-(9-(2-(((S)-2,6-dioxopiperidin-3-yl)carbamoyl)pyridin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

To a cooled (0° C.) solution of (R)-4-(9-(3-methyl-6-((2-(1-methylpyrrolidin-2-yl)-1H-pyrrolo[3,2-c]pyridin-6-yl)carbamoyl)-1H-indazol-1-yl)non-1-yn-1-yl)picolinic acid trifluoroacetate (Intermediate 192, 0.9 g, 1.45 mmol) and (S)-3-aminopiperidine-2,6-dione hydrochloride (CAS No: 25181-50-4, 0.24 g, 1.45 mmol) in dimethylformamide (8 mL) were added HATU (0.66 g, 1.74 mmol) and DIPEA (0.93 g, 7.25 mmol). The reaction mixture was stirred at room temperature for 16 h. The mixture was poured into ice cold water (50 mL), the resultant precipitate collected by filtration and dried in vacuo to afford the crude product which was purified by preparative HPLC (Method A) to afford 1-(9-(2-(((S)-2,6-dioxopiperidin-3-yl)carbamoyl)pyridin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 43, 0.10 g, 9%) as an off-white solid. ¹H NMR (400 MHz, DMSO-d₆) δ ppm 1.30-1.39 (m, 6H), 1.53-1.57 (m, 2H), 1.81-1.87 (m, 5H), 1.89-1.98 (m, 1H), 2.20 (s, 3H), 2.46-2.49 (m, 3H), 2.51-2.53 (m, 2H), 2.54 (s, 3H), 2.55 (m, 1H), 2.58-2.59 (m, 1H), 3.14-3.17 (m, 1H), 3.30-3.34 (m, 1H), 4.41 (t, J=6.8 Hz, 2H), 4.77-4.81 (m, 1H), 6.40 (s, 1H), 7.54-7.56 (m, 1H), 7.70-7.72 (m, 2H), 7.89 (d, J=4.0 Hz, 1H), 8.24 (s, 1H), 8.44 (s, 1H), 8.51 (s, 1H), 8.61 (s, 1H), 9.08 (d, J=8.4 Hz, 1H), 10.53 (s, 1H), 10.87 (s, 1H), 11.38 (s, 1H). Mass spec m/z 728.10 [M+H]⁺.

### Example 44 was Synthesized Following Scheme 44

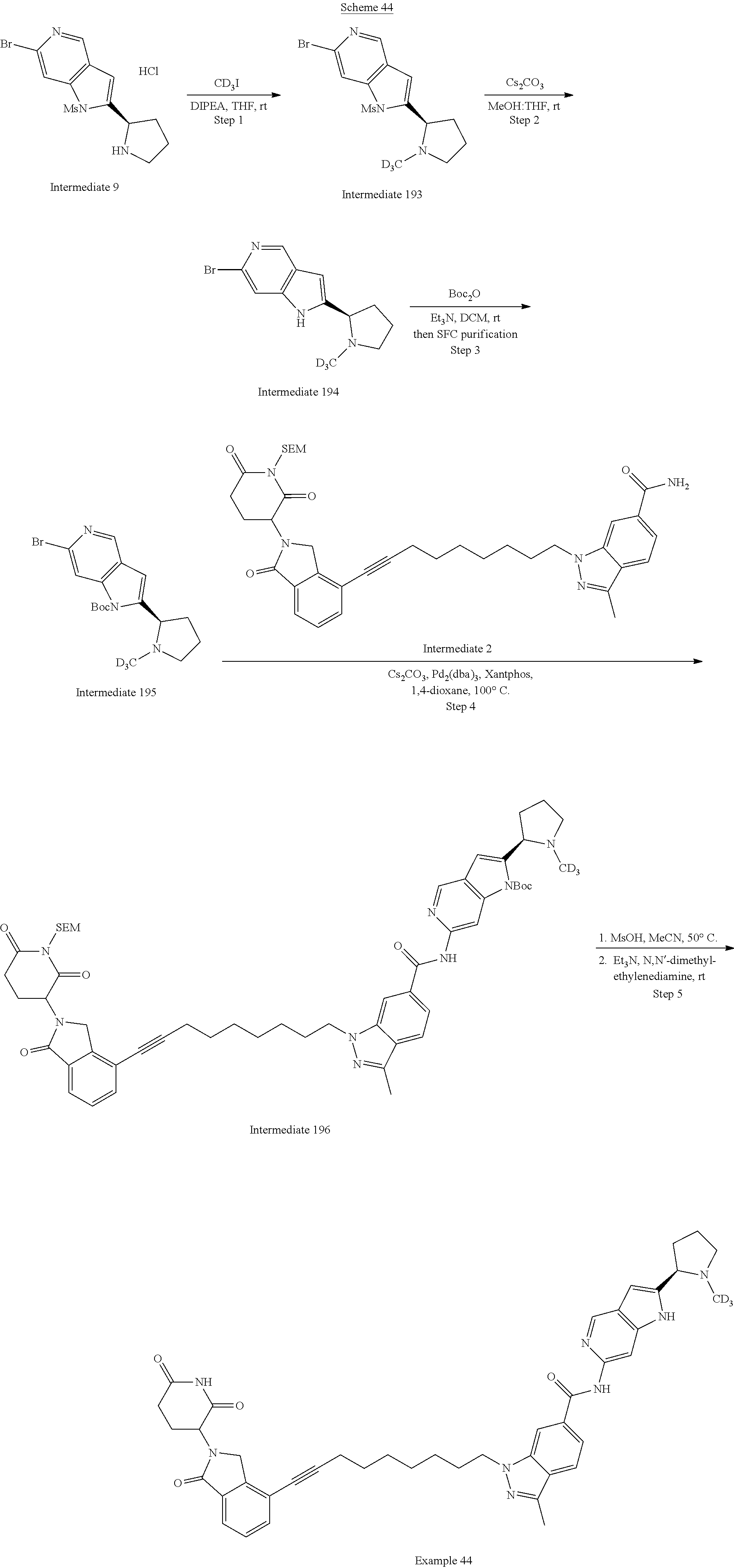

### Step 1

### Intermediate 193: (R)-6-bromo-2-(1-(methyl-d3) pyrrolidin-2-yl)-1-(methylsulfonyl)-1H-pyrrolo [3,2-c]pyridine

To a cooled (0° C.) solution of (R)-6-bromo-1-(methylsulfonyl)-2-(pyrrolidin-2-yl)-1H-pyrrolo[3,2-c]pyridine hydrochloride (Intermediate 9, 10.0 g, 29.1 mmol) in tetrahydrofuran (40 mL) was added DIPEA (15.8 g, 116 mmol) followed by CD₃I (6.32 g, 43.6 mmol). The reaction mixture was stirred at room temperature for 16 h. The reaction mixture was poured into water (100 mL) and extracted with ethyl acetate (3×150 mL). The organic layer was separated, dried over anhydrous Na₂SO₄, filtered, and concentrated in vacuo. The crude material was purified by combi-flash chromatography, by eluting with 80% ethyl acetate in heptane, to afford (R)-6-bromo-2-(1-(methyl-d3)pyrrolidin-2-yl)-1-(methylsulfonyl)-1H-pyrrolo[3,2-c]pyridine (Intermediate 193, 5.2 g, 50%) as a yellow solid. ¹H NMR (400 MHz, DMSO-d₆) δ ppm 1.68-1.80 (m, 3H), 2.29-2.38 (m, 2H), 3.10-3.12 (m, 1H), 3.56 (s, 3H), 3.77-3.81 (m, 1H), 6.89 (s, 1H), 7.96 (s, 1H), 8.68 (s, 1H). Mass spec m/z 362.8 [M+H+2]⁺.

### Step 2

### Intermediate 194: (R)-6-bromo-2-(1-(methyl-d3)pyrrolidin-2-yl)-1H-pyrrolo[3,2-c]pyridine

To a cooled (0° C.) solution of (R)-6-bromo-2-(1-(methyl-d3) pyrrolidin-2-yl)-1-(methylsulfonyl)-1H-pyrrolo [3,2-c]pyridine (Intermediate 193, 5.0 g, 14 mmol) in tetrahydrofuran (20 mL) and methanol (20 mL) was added cesium carbonate (9.0 g, 28 mmol), then the reaction mixture was stirred at room temperature for 2 h. The reaction mixture was poured into ice cold water (100 mL) and extracted with dichloromethane (3×100 mL). The combined organic layers were dried over anhydrous Na₂SO₄, filtered, and concentrated in vacuo to afford (R)-6-bromo-2-(1-(methyl-d3) pyrrolidin-2-yl)-1H-pyrrolo[3,2-c]pyridine (Intermediate 194, 3.2 g, 77%) as an off-white solid which was used for the next step without further purification. ¹H NMR (400 MHz, DMSO-d₆) δ ppm 1.70-1.94 (m, 3H), 2.12-2.19 (m, 1H), 2.23-2.30 (m, 1H), 3.13-3.15 (m, 1H), 3.32-3.38 (m, 1H), 6.45 (s, 1H), 7.43 (s, 1H), 8.48 (s, 1H), 11.57 (br s, 1H).

### Step 3

### Intermediate 195: tert-butyl (R)-6-bromo-2-(1-(methyl-d3) pyrrolidin-2-yl)-1H-pyrrolo [3,2-c]pyridine-1-carboxylate

To a solution of (R)-6-bromo-2-(1-(methyl-d3) pyrrolidin-2-yl)-1H-pyrrolo [3,2-c]pyridine (Intermediate 194, 3.0 g, 11 mmol) in dichloromethane (30 mL) was added triethylamine (3.2 g, 32 mmol) followed by di-tert-butyl dicarbonate (3.5 g, 16 mmol) and 4-dimethylaminopyridine (0.13 g, 1.1 mmol) and the reaction mixture was stirred at room temperature for 16 h. The reaction mixture was poured into water (100 mL) and extracted with dichloromethane (3×100 mL). The combined organic layers were dried over anhydrous Na₂SO₄, filtered, and concentrated in vacuo to obtain the crude compound. The crude material was purified by combi-flash chromatography, by eluting with 50% ethyl acetate in heptane, to afford tert-butyl (R)-6-bromo-2-(1-(methyl-d3)pyrrolidin-2-yl)-1H-pyrrolo[3,2-c]pyridine-1-carboxylate (2.5 g) as an off white solid. This material was further purified by SFC chiral purification (Column: Chiralpak IK (250*30 mm) 5 um. Isocratic elution with 25% Mobile Phase (A): CO₂; mobile Phase (B): MeCN+isopropylalcohol) to afford (R)-6-bromo-2-(1-(methyl-d3) pyrrolidin-2-yl)-1H-pyrrolo[3,2-c]pyridine-1-carboxylate (Intermediate 195, 1.8 g, 44%) as an off white solid. ¹H NMR (400 MHz, DMSO-d₆) δ ppm 1.65 (s, 9H), 1.69-1.81 (m, 3H), 2.31-2.36 (m, 2H), 3.07-3.14 (m, 1H), 3.85-3.62 (m, 1H), 6.79 (s, 1H), 8.04 (s, 1H), 8.60 (s, 1H). Mass spec m/z 384.9 [M+H+2]⁺.

### Step 4

### Intermediate 196: tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((R)-1-(methyl-d3)pyrrolidin-2-yl)-1H-pyrrolo[3,2-c]pyridine-1-carboxylate

To a solution of 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 2, 0.56 g, 0.83 mmol) and tert-butyl (R)-6-bromo-2-(1-(methyl-d3)pyrrolidin-2-yl)-1H-pyrrolo[3,2-c]pyridine-1-carboxylate (Intermediate 195, 0.40 g, 1.04 mmol) in 1,4-dioxane (20 mL) was added cesium carbonate (0.68 g, 2.08 mmol). The reaction mixture was purged with argon gas for 20 min then Xantphos (0.18 g, 0.31 mmol) and Pd₂(dba)₃ (0.15 g, 0.15 mmol) were added. The reaction mixture was purged with argon gas for 10 min and then heated at 100° C. for 2h. The reaction mixture was concentrated in vacuo and the crude material was purified by combi-flash chromatography, by eluting with 80% ethyl acetate in heptane, to afford tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((R)-1-(methyl-d3)pyrrolidin-2-yl)-1H-pyrrolo[3,2-c]pyridine-1-carboxylate (Intermediate 196, 0.39 g, 38%) as a yellow solid. ¹H NMR (400 MHz, DMSO-d₆) δ ppm −0.06 (s, 9H), 0.79-0.89 (m, 2H), 1.27-1.38 (m, 8H), 1.51-1.54 (m, 2H), 1.70 (s, 9H), 1.74-1.80 (m, 2H), 1.86 (s, 3H), 2.33-2.45 (m, 4H), 2.67-2.79 (m, 2H), 3.01-3.13 (m, 2H), 3.38-3.40 (m, 2H), 3.48-3.50 (m, 2H), 3.87-3.92 (m, 1H), 4.21-4.24 (m, 1H), 4.40 (s, 2H), 4.42-4.56 (m, 2H), 4.99-5.07 (m, 1H), 5.24-5.28 (m, 1H), 6.76 (s, 1H), 7.49-7.59 (m, 2H), 7.71-7.80 (m, 3H), 8.42 (s, 1H), 8.60 (s, 1H), 8.97 (s, 1H), 10.75 (br s, 1H). Mass spec m/z 972.2 [M+H]⁺.

### Step 5

### Example 44: 1-(9-(2-(2,6-dioxopiperidin-3-yl)-1-oxoisoindolin-4-yl)non-8-yn-1-yl)-3-methyl-N-(2-((R)-1-(methyl-d3)pyrrolidin-2-yl)-1H-pyrrolo[3,2-c]pyridin-6-yl)-1H-indazole-6-carboxamide

To a solution of tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((R)-1-(methyl-d3)pyrrolidin-2-yl)-1H-pyrrolo[3,2-c]pyridine-1-carboxylate (Intermediate 196, 0.38 g, 0.39 mmol) in acetonitrile (10 mL) was added methanesulfonic acid (0.38 g, 3.90 mmol) and the reaction mixture was heated at 50° C. for 2 h. The reaction mixture was cooled to room temperature then N,N′-dimethylethylenediamine (0.19 g, 1.95 mmol) followed by triethylamine (0.79 g, 7.81 mmol) were added and the mixture stirred for 3 h at room temperature. The reaction mixture was concentrated in vacuo and poured into water (20 mL). The resultant precipitate was collected by filtration and dried in vacuo to afford the crude compound. The crude material was purified by preparative HPLC (Method A) to afford to afford 1-(9-(2-(2,6-dioxopiperidin-3-yl)-1-oxoisoindolin-4-yl)non-8-yn-1-yl)-3-methyl-N-(2-((R)-1-(methyl-d3)pyrrolidin-2-yl)-1H-pyrrolo[3,2-c]pyridin-6-yl)-1H-indazole-6-carboxamide (Example 44, 0.025 g, 9%) as an off white solid. ¹H NMR (400 MHz, DMSO-d₆) δ ppm 1.32-1.39 (m, 6H), 1.52-1.56 (m, 2H), 1.78-1.92 (m, 5H), 1.98-2.03 (m, 1H), 2.14-2.17 (m, 1H), 2.25-2.28 (m, 1H), 2.41-2.48 (m, 5H), 2.54-2.59 (m, 3H), 2.84-2.95 (m, 1H), 3.12-3.16 (m, 1H), 4.26-4.31 (m, 1H), 4.38-4.45 (m, 3H), 5.10-5.15 (m, 1H), 6.40 (s, 1H), 7.49 (t, J=7.57 Hz, 1H), 7.59 (d, J=6.88 Hz, 1H), 7.66-7.75 (m, 2H), 7.75-7.79 (m, 1H), 8.25 (s, 1H), 8.43 (s, 1H), 8.51 (s, 1H), 10.51 (s, 1H), 11.00 (br s, 1H), 11.38 (br s, 1H). Mass spec m/z 742.3 [M+H]⁺.

### Example 45 was Synthesized Following Scheme 45

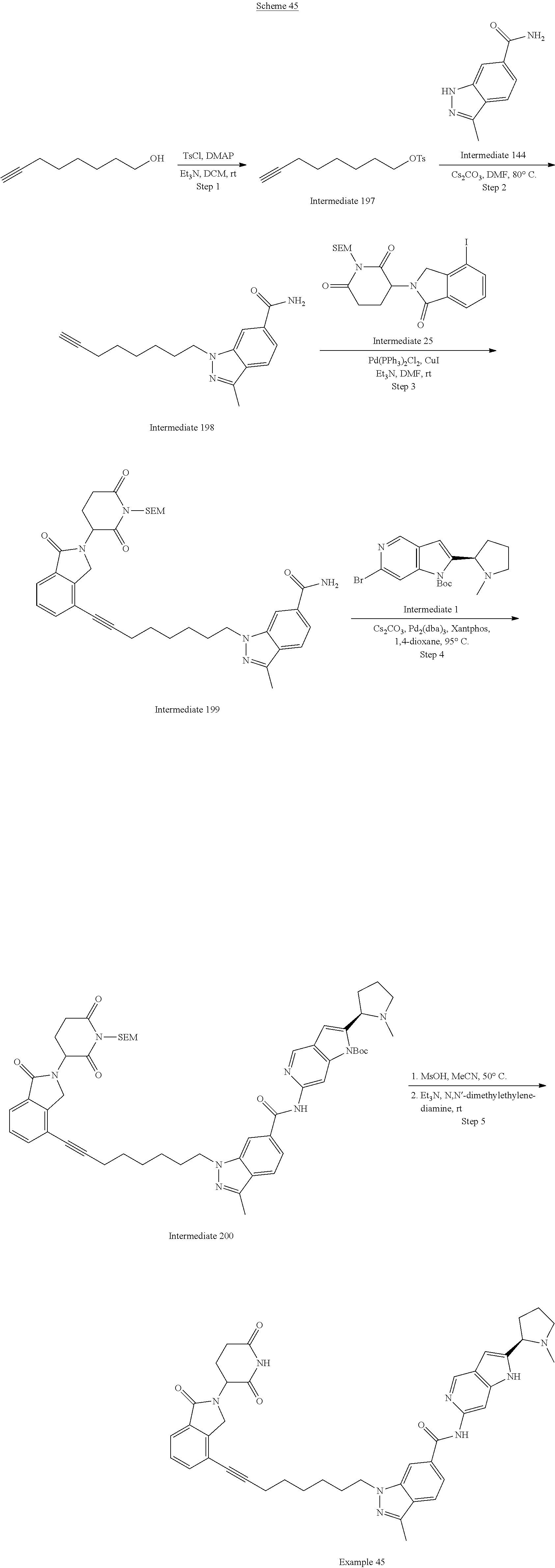

### Step 1

### Intermediate 197: oct-7-yn-1-yl 4-methylbenzenesulfonate

To a cooled (0° C.) solution of oct-7-yn-1-ol (CAS No: 871-91-0, 5.0 g, 40 mmol) in dichloromethane (20 mL) was added triethylamine (13 g, 120 mmol) followed by p-toluenesulfonyl chloride (8.8 mL, 59 mmol) and 4-dimethylaminopyridine (0.51 g, 4.0 mmol). The reaction mixture was stirred at room temperature for 16 h then poured into ice cold water (50 mL) and extracted with DCM (2×100 mL). The combined organic layers were dried over anhydrous Na₂SO₄, filtered, and concentrated in vacuo. The crude material was purified by combi-flash chromatography, by eluting with 50% ethyl acetate in heptane, to afford oct-7-yn-1-yl 4-methylbenzenesulfonate (Intermediate 197, 7.2 g, 65%) as a yellow liquid. ¹H NMR (400 MHz, CDCl₃) δ ppm 1.32-1.38 (m, 4H), 1.43-1.50 (m, 2H), 1.61-1.66 (m, 2H), 1.92 (s, 1H), 2.12-2.16 (m, 2H), 2.45 (s, 3H), 4.02 (t, J=6.47 Hz, 2H), 7.34 (d, J=7.89 Hz, 2H), 7.79 (d, J=8.40 Hz, 2H).

### Step 2

### Intermediate 198: 3-methyl-1-(oct-7-yn-1-yl)-1H-indazole-6-carboxamide

A mixture of oct-7-yn-1-yl 4-methylbenzenesulfonate (Intermediate 197, 5.76 g, 20.5 mmol), 3-methyl-1H-indazole-6-carboxamide (Intermediate 144, 3.00 g, 17.1 mmol) and cesium carbonate (17.6 g, 51.4 mmol) in dimethylformamide (60 mL) was heated at 80° C. for 3 h. The reaction mixture was poured into ice cold water (100 mL) and extracted with ethyl acetate (2×150 mL). The combined organic layers were dried over anhydrous Na₂SO₄, filtered, and concentrated in vacuo to obtain crude compound as a mixture of regioisomers. The regioisomer were separated by combi-flash chromatography, by eluting with 70% ethyl acetate in heptane, to afford desired regioisomer 3-methyl-1-(oct-7-yn-1-yl)-1H-indazole-6-carboxamide (Intermediate 198, 2.0 g, 41%) as an off white solid. ¹H NMR (400 MHz, DMSO-d₆) δ ppm 1.22-1.27 (m, 2H), 1.35-1.45 (m, 4H), 1.78-1.85 (m, 2H), 2.06-2.16 (m, 2H), 2.50 (s, 3H), 2.75 (s, 1H) 4.33 (t, J=7.05 Hz, 2H), 7.42 (br s, 1H), 7.59 (d, J=8.29 Hz, 1H), 7.73 (d, J=8.29 Hz, 1H). 8.03 (br s, 1H), 8.12 (s, 1H). Mass spec m/z 284.1 [M+H]⁺.

### Step 3

### Intermediate 199: 1-(8-(2-(2,6-dioxo-1-((2-(trimethylsilyl) ethoxy) methyl) piperidin-3-yl)-1-oxoisoindolin-4-yl) oct-7-yn-1-yl)-3-methyl-1H-indazole-6-carboxamide

To a solution of 3-methyl-1-(oct-7-yn-1-yl)-1H-indazole-6-carboxamide (Intermediate 198, 0.57 g, 2.0 mmol) and 3-(4-iodo-1-oxo-isoindolin-2-yl)-1-(2-trimethylsilylethoxymethyl)piperidine-2,6-dione (Intermediate 25, 1.0 g, 2.0 mmol) in dimethylformamide (15 mL) was added triethylamine (0.83 g, 7.8 mmol). The reaction mixture was purged with argon gas for 15 min then Pd(PPh₃)₂Cl₂ (0.15 g, 0.20 mmol) and copper (I) iodide (0.040 g, 0.20 mmol) were added. The reaction mixture was purged with argon gas for a further 10 min then stirred at room temperature for 1 h. The reaction mixture was poured into water (90 mL) and extracted with ethyl acetate (2×80 mL). The combined organic layers were dried over anhydrous Na₂SO₄, filtered, and concentrated in vacuo. The crude material was purified by combi-flash chromatography, by eluting with 95% ethyl acetate in heptane, to afford 1-(8-(2-(2,6-dioxo-1-((2-(trimethylsilyl)ethoxy)methyl)piperidin-3-yl)-1-oxoisoindolin-4-yl)oct-7-yn-1-yl)-3-methyl-1H-indazole-6-carboxamide (Intermediate 199, 1.20 g, 92%) as a brown solid. ¹H NMR (400 MHz, DMSO-d₆) δ ppm −0.05 (s, 9H), 0.79-0.85 (m, 2H), 1.27-1.29 (m, 2H), 1.45-1.56 (m, 4H), 1.79-1.83 (m, 2H), 1.99-2.07 (m, 2H), 2.37 (s, 3H), 2.67-2.77 (m, 2H), 2.87-2.89 (m, 2H), 3.02-3.07 (m, 1H), 3.46-3.52 (m, 2H), 4.23-4.48 (m, 4H), 4.98-5.06 (m, 2H), 5.24-5.27 (m, 1H), 7.50-7.53 (m, 1H), 7.56-7.63 (m, 2H), 7.71 (br s, 2H), 7.95-8.03 (m, 1H), 8.12 (s, 1H). Mass spec m/z 656.51 [M+H]⁺.

### Step 4

### Intermediate 200: tert-butyl 6-(1-(8-(2-(2,6-dioxo-1-((2-(trimethylsilyl)ethoxy)methyl)piperidin-3-yl)-1-oxoisoindolin-4-yl)oct-7-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(8-(2-(2,6-dioxo-1-((2-(trimethylsilyl)ethoxy)methyl)piperidin-3-yl)-1-oxoisoindolin-4-yl)oct-7-yn-1-yl)-3-methyl-1H-indazole-6-carboxamide (Intermediate 199, 1.10 g, 1.68 mmol) and tert-butyl (R)-6-bromo-2-(1-methylpyrrolidin-2-yl)-1H-pyrrolo[3,2-c]pyridine-1-carboxylate (Intermediate 1, 0.80 g, 2.10 mmol) in 1,4-dioxane (10 mL) was added cesium carbonate (1.44 g, 4.21 mmol). The reaction mixture was purged with argon gas for 15 min then Xantphos (0.38 g, 0.63 mmol) and Pd₂(dba)₃ (0.26 g, 0.31 mmol) were added. The reaction mixture was purged with argon gas for 10 min and then heated at 95° C. for 1 h. The reaction mixture was filtered through the a celite bed, washed with dichloromethane (20 mL) and the filtrate was concentrated in vacuo. The crude material was purified by combi-flash chromatography, by eluting with 10% MeOH in DCM, to afford tert-butyl 6-(1-(8-(2-(2,6-dioxo-1-((2-(trimethylsilyl)ethoxy)methyl)piperidin-3-yl)-1-oxoisoindolin-4-yl)oct-7-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 200, 1.0 g, 50%) as a brown solid. ¹H NMR (400 MHz, DMSO-d₆) δ ppm −0.06 (s, 9H), 0.76-0.83 (m, 2H), 1.27-1.38 (m, 2H), 1.47-1.57 (m, 5H), 1.70 (s, 9H), 1.74-1.77 (m, 2H), 1.83-1.91 (m, 3H), 1.99-2.06 (m, 2H), 2.36 (s, 3H), 2.43-2.46 (m, 1H), 2.47 (s, 3H), 2.67-2.75 (m, 3H), 2.87-2.89 (m, 1H), 2.99-3.08 (m, 2H), 3.12-3.16 (m, 1H), 3.46-3.51 (m, 2H), 3.90-3.94 (m, 1H), 4.22-4.26 (m, 1H), 4.39-4.46 (m, 2H), 4.95-5.06 (m, 1H), 5.22-5.26 (m, 1H), 6.76 (s, 1H), 7.48-7.52 (m, 1H), 7.59-7.62 (m, 1H), 7.69-7.74 (m, 2H), 7.75-7.81 (m, 1H), 8.42 (s, 1H), 8.60 (s, 1H), 8.97 (s, 1H), 10.75 (s, 1H). Mass spec m/z 955.1 [M+H]⁺.

### Step 5

### Example 45: 1-(8-(2-(2,6-dioxopiperidin-3-yl)-1-oxoisoindolin-4-yl)oct-7-yn-1-yl)-3-methyl-N-(2-((R)-1-methylpyrrolidin-2-yl)-1H-pyrrolo[3,2-c]pyridin-6-yl)-1H-indazole-6-carboxamide

To a cooled (0° C.) solution of tert-butyl 6-(1-(8-(2-(2,6-dioxo-1-((2-(trimethylsilyl)ethoxy)methyl)piperidin-3-yl)-1-oxoisoindolin-4-yl)oct-7-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 200, 0.50 g, 0.52 mmol) in acetonitrile (5 mL) was added methanesulfonic acid (0.53 g, 5.23 mmol) and the reaction mixture was heated at 50° C. for 2h. The reaction mixture was cooled to room temperature then N,N′-dimethylethylenediamine (0.24 g, 2.62 mmol) followed by triethylamine (1.11 g, 10.47 mmol) were added and the reaction stirred at room temperature for 16 h. The reaction mixture was concentrated in vacuo and the crude material was poured into water (60 mL). The resultant precipitate was collected by filtration and dried in vacuo to afford the crude product which was purified by preparative HPLC (Method A) to afford 1-(8-(2-(2,6-dioxopiperidin-3-yl)-1-oxoisoindolin-4-yl)oct-7-yn-1-yl)-3-methyl-N-(2-((R)-1-methylpyrrolidin-2-yl)-1H-pyrrolo[3,2-c]pyridin-6-yl)-1H-indazole-6-carboxamide (Example 45, 0.10 g, 27%) as an off white solid. ¹H NMR (400 MHz, DMSO-d₆) δ ppm 1.30-1.37 (m, 2H), 1.44-1.60 (m, 4H), 1.77-2.00 (m, 7H), 2.13-2.15 (m, 1H), 2.17 (s, 3H), 2.23-2.30 (m, 1H), 2.40-2.47 (m, 3H), 2.51 (s, 3H), 2.55-2.60 (m, 1H), 2.84-2.93 (m, 1H), 3.11-3.18 (m, 1H), 4.27-4.32 (m, 1H), 4.40-4.46 (m, 3H), 5.10-5.14 (m, 1H), 6.40 (s, 1H), 7.46-7.52 (m, 1H), 7.61 (m, 1H), 7.69 (m, 1H), 7.71-7.75 (m, 1H), 7.76-7.80 (m, 1H), 8.25 (s, 1H), 8.43 (s, 1H), 8.52 (s, 1H), 10.54 (s, 1H), 10.99 (s, 1H), 11.39 (s, 1H). Mass spec m/z 725.1 [M+H]⁺.

### Example 46 was Synthesized Following Scheme 46

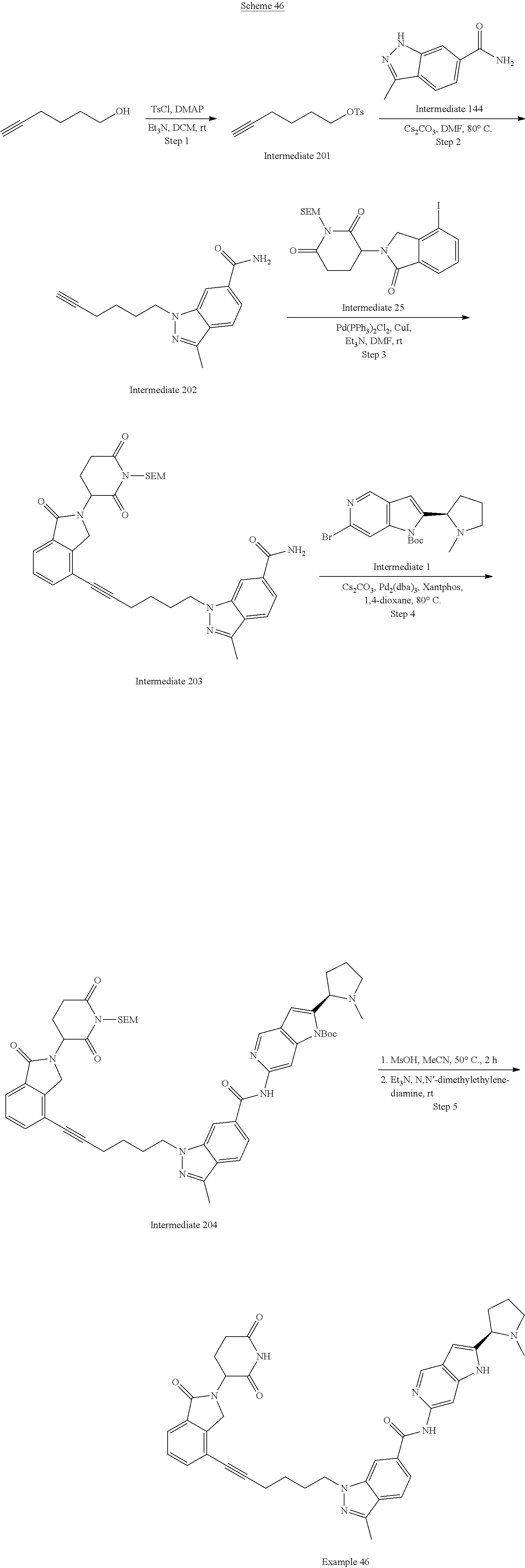

### Step 1

### Intermediate 201: hex-5-yn-1-yl 4-methylbenzenesulfonate

To solution of hex-5-yn-1-ol (CAS No: 928-90-5, 5.00 g, 51 mmol) in dichloromethane (50 mL) was added triethylamine (21 mL, 150 mmol) at 0° C. followed by p-toluenesulfonyl chloride (15 g, 76 mmol) and 4-dimethylaminopyridine (0.66 g, 5.1 mmol). The reaction mixture was stirred at room temperature for 16 h. The reaction mixture was diluted with water (25 mL) and extracted with dichloromethane (3×25 mL). The organic layer was separated, dried over anhydrous Na₂SO₄, filtered, and concentrated in vacuo. The crude material was purified by combi-flash chromatography, by eluting with 55% ethyl acetate in heptane, to afford hex-5-yn-1-yl 4-methylbenzenesulfonate (Intermediate 201, 7.00 g, 54%) as a yellow oil. ¹H NMR (400 MHz, DMSO-d₆) δ ppm 1.37-1.44 (m, 2H), 1.61-1.68 (m, 2H), 2.08-2.13 (m, 2H), 2.42 (s, 3H), 2.74 (t, J=2.4 Hz, 1H), 4.04 (t, J=6.40 Hz, 2H), 7.48 (d, J=8.40 Hz, 2H), 7.78 (d, J=8.40 Hz, 2H).

### Step 2

### Intermediate 202: 1-(hex-5-yn-1-yl)-3-methyl-1H-indazole-6-carboxamide

To a solution of hex-5-yn-1-yl 4-methylbenzenesulfonate (Intermediate 201, 2.9 g, 11 mmol) in dimethylformamide (2.0 mL) was added 3-methyl-1H-indazole-6-carboxamide (Intermediate 144, 2.0 g, 11 mmol) followed by cesium carbonate (11 g, 34 mmol) at room temperature. The reaction mixture was heated at 80° C. for 6 h. The reaction mixture was diluted with water (100 mL) and extracted with ethyl acetate (3×100 mL). The organic layer was washed with brine solution (2×60 mL), separated, dried over anhydrous Na₂SO₄, filtered, and concentrated in vacuo to obtain the crude compound as a mixture of regioisomers which were separated by combi-flash chromatography, by eluting with 50% ethyl acetate in heptane, to afford the desired regioisomer 1-(hex-5-yn-1-yl)-3-methyl-1H-indazole-6-carboxamide (Intermediate 202, 1.50 g, 51%) as a white solid. ¹H NMR (400 MHz, DMSO-d₆) δ ppm 1.42-1.49 (m, 2H), 1.92-1.99 (m, 2H), 2.18-2.22 (m, 2H), 2.63 (s, 3H), 2.77 (t, J=2.4 Hz, 1H), 4.39 (t, J=6.8 Hz, 2H), 7.28 (br s, 1H), 7.45 (d, J=8.80 Hz, 1H), 7.69 (d, J=8.71 Hz, 1H), 7.96 (br s, 1H), 8.12 (s, 1H). Mass spec m/z 256.0 [M+H]⁺.

### Step 3

### Intermediate 203: 1-(6-(2-(2,6-dioxo-1-((2-(trimethylsilyl)ethoxy)methyl)piperidin-3-yl)-1-oxoisoindolin-4-yl) hex-5-yn-1-yl)-3-methyl-1H-indazole-6-carboxamide

To a solution of 1-(hex-5-yn-1-yl)-3-methyl-1H-indazole-6-carboxamide (Intermediate 202, 1.00 g, 3.9 mmol) in dimethylformamide (10 mL) was added 3-(4-iodo-1-oxoisoindolin-2-yl)-1-((2-(trimethylsilyl)ethoxy)methyl)piperidine-2,6-dione (Intermediate 25, 2.00 g, 3.9 mmol) followed by triethylamine (19 mL, 140 mmol) at room temperature. The reaction mixture was purged with argon gas for 15 min then Pd Cl₂(PPh₃)₂ (0.28 g, 0.39 mmol) and copper (I) iodide (0.076 g, 0.39 mmol) were added at room temperature. The reaction mixture was purged with argon gas for another 10 min and stirred at room temperature for 2 h. The reaction mixture was diluted with water (100 mL) and extracted with ethyl acetate (3×200 mL). The organic layer was separated, dried over anhydrous Na₂SO₄, filtered, and concentrated in vacuo. The crude material was purified by combi-flash chromatography, by eluting with 100% ethyl acetate in heptane, to afford 1-(6-(2-(2,6-dioxo-1-((2-(trimethylsilyl)ethoxy)methyl)piperidin-3-yl)-1-oxoisoindolin-4-yl)hex-5-yn-1-yl)-3-methyl-1H-indazole-6-carboxamide (Intermediate 203, 1.50 g, 61%) as a yellow solid. ¹H NMR (400 MHz, DMSO-d₆) δ ppm −0.05 (s, 9H), 0.77-0.86 (m, 2H), 1.51-1.58 (m, 2H), 1.96-2.08 (m, 3H), 2.32-2.39 (m, 1H), 2.48 (s, 3H), 2.72-2.80 (m, 2H), 2.88-2.90 (m, 1H), 3.02-3.12 (m, 1H), 3.46-3.56 (m, 2H), 4.22-4.26 (m, 1H), 4.40-4.46 (m, 3H), 5.00-5.07 (m, 2H), 5.23-5.27 (m, 1H), 7.42 (br s, 1H), 7.49-7.54 (m, 1H), 7.58-7.62 (m, 2H), 7.70-7.74 (m, 2H), 8.03 (br s, 1H), 8.16 (s, 1H). Mass spec m/z 628.3 [M+H]⁺.

### Step 4

### Intermediate 204: tert-butyl 6-(1-(6-(2-(2,6-dioxo-1-((2-(trimethylsilyl)ethoxy)methyl)piperidin-3-yl)-1-oxoisoindolin-4-yl)hex-5-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(6-(2-(2,6-dioxo-1-((2-(trimethylsilyl)ethoxy)methyl)piperidin-3-yl)-1-oxoisoindolin-4-yl)hex-5-yn-1-yl)-3-methyl-1H-indazole-6-carboxamide (Intermediate 203, 0.90 g, 1.4 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 1, 0.65 g, 1.7 mmol) followed by cesium carbonate (1.40 g, 4.3 mmol). The reaction mixture was purged with argon gas for 15 min then Xantphos (0.26 g, 0.43 mmol) and Pd₂ (dba)₃ (0.20 g, 0.22 mmol) were added at room temperature. The reaction mixture was further purged with argon gas for 10 min and heated at 80° C. for 2 h. The reaction mixture was concentrated in vacuo to obtain crude compound. The crude material was purified by combi-flash chromatography, by eluting with 100% ethyl acetate in heptane, to afford tert-butyl 6-(1-(6-(2-(2,6-dioxo-1-((2-(trimethylsilyl)ethoxy)methyl)piperidin-3-yl)-1-oxoisoindolin-4-yl)hex-5-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 204, 0.50 g, 40%) as a yellow solid. ¹H NMR (400 MHz, DMSO-d₆) δ ppm −0.06 (s, 9H), 0.76-0.83 (m, 2H), 1.19-1.23 (m, 2H), 1.47-1.57 (m, 6H), 1.70 (s, 9H), 2.04 (s, 3H), 2.36 (s, 3H), 2.43-2.47 (m, 4H), 2.66-2.78 (m, 2H), 3.04-3.14 (m, 2H), 3.46-3.51 (m, 2H), 3.84-4.10 (m, 2H), 4.22-4.26 (m, 2H), 4.39-4.46 (m, 2H), 4.95-5.06 (m, 1H), 5.22-5.26 (m, 1H), 6.76 (s, 1H), 7.11-7.54 (m, 2H), 7.56-7.86 (m, 3H), 8.46 (s, 1H), 8.60 (s, 1H), 8.97 (s, 1H), 10.74 (br s, 1H). Mass spec m/z 927.2 [M+H]⁺.

### Step 5

### Example 46: 1-(6-(2-(2,6-dioxopiperidin-3-yl)-1-oxoisoindolin-4-yl)hex-5-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(6-(2-(2,6-dioxo-1-((2-(trimethylsilyl)ethoxy)methyl)piperidin-3-yl)-1-oxoisoindolin-4-yl)hex-5-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 204, 0.50 g, 0.5 mmol) in acetonitrile (8 mL) was added methanesulfonic acid (0.5 mL, 8 mmol) at room temperature and the reaction mixture was heated at 50° C. for 2 h. After cooling to room temperature, N,N′-dimethylethylenediamine (0.4 mL, 3 mmol) followed by triethylamine (2 mL, 10 mmol) were added and stirred for 3 h at room temperature. The reaction mixture was concentrated in vacuo to obtain crude compound which was poured into ice cold water (10 mL), and the resultant solid precipitate collected by filtration and dried in vacuo to obtain crude compound. The crude material was purified by preparative HPLC (Method A) to afford 1-(6-(2-(2,6-dioxopiperidin-3-yl)-1-oxoisoindolin-4-yl)hex-5-yn-1-yl)-3-methyl-N-(2-((R)-1-methylpyrrolidin-2-yl)-1H-pyrrolo[3,2-c]pyridin-6-yl)-1H-indazole-6-carboxamide (Example 46, 0.062 g, 20%) as an off white solid. ¹H NMR (400 MHz, DMSO-d₆) δ ppm 1.55-1.62 (m, 2H), 1.77-1.93 (m, 3H), 1.95-2.08 (m, 4H), 2.10-2.15 (m, 1H), 2.17 (s, 3H), 2.24-2.30 (m, 1H), 2.37-2.42 (m, 1H), 2.48 (s, 3H), 2.53-2.62 (m, 3H), 2.85-2.95 (m, 1H), 3.12-3.18 (m, 1H), 4.26-4.30 (m, 1H), 4.38-4.41 (m, 1H), 4.45-4.50 (m, 2H), 5.09-5.13 (m, 1H), 6.40 (s, 1H), 7.46-7.49 (m, 1H), 7.59-7.61 (m, 1H), 7.67-7.69 (m, 1H), 7.72-7.76 (m, 1H), 7.77-7.81 (m, 1H), 8.24 (s, 1H), 8.47 (s, 1H), 8.52 (s, 1H), 10.53 (s, 1H), 10.98 (br s, 1H), 11.39 (br s, 1H). Mass spec m/z 697.1 [M+H]⁺.

### Example 47: 1-(5-(4-(2-(2,6-dioxopiperidin-3-yl)-1,3-dioxoisoindolin-4-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide

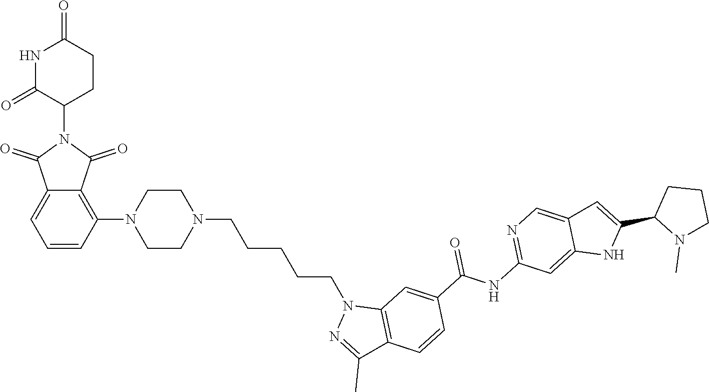 [SMILES: Cc1nn(CCCCCN2CCN(c3cccc4c3C(=O)N(C3CCC(=O)NC3=O)C4=O)CC2)c2cc(C(=O)Nc3cc4[nH]c([C@H]5CCCN5C)cc4cn3)ccc12]

To a solution of(R)-3-methyl-N-(2-(1-methylpyrrolidin-2-yl)-1H-pyrrolo[3,2-c]pyridin-6-yl)-1-(5-(piperazin-1-yl)pentyl)-1H-indazole-6-carboxamide dihydrochloride (Intermediate 111, 0.40 g, 0.75 mmol) in DMSO (7 mL) was added DIPEA (0.41 g, 3.02 mmol) followed by 2-(2,6-dioxopiperidin-3-yl)-4-fluoroisoindoline-1,3-dione (CAS No: 835616-60-9, 0.20 g, 0.75 mmol) and the reaction mixture was heated at 100° C. for 1 h. The reaction mixture was poured into ice cold water and the resultant precipitate was collected by filtration and dried in vacuo. The crude compound was purified by preparative HPLC (Method A) to afford 1-(5-(4-(2-(2,6-dioxopiperidin-3-yl)-1,3-dioxoisoindolin-4-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide (Example 47, 0.10 g, 17%) as an off-white solid. ¹H NMR (400 MHz, DMSO-d₆) δ ppm 1.30-1.40 (m, 2H), 1.50-1.65 (m, 2H), 1.76-1.95 (m, 6H), 1.95-2.05 (m, 1H), 2.16-2.17 (m, 1H), 2.17 (s, 3H), 2.23-2.32 (m, 3H), 2.50-2.52 (m, 2H), 2.53 (s, 3H), 2.54-2.60 (m, 3H), 2.80-2.94 (m, 1H), 3.10-3.19 (m, 2H), 3.20-3.24 (m, 4H), 4.43-4.55 (m, 2H), 5.07-5.40 (m, 1H), 6.40 (s, 1H), 7.23 (d, J=8.44 Hz, 1H), 7.32 (d, J=7.09 Hz, 1H), 7.63-7.70 (m, 1H), 7.73 (d, J=8.41 Hz, 1H), 7.79 (d, J=7.87 Hz, 1H), 8.25 (s, 1H), 8.47 (s, 1H), 8.52 (s, 1H), 10.55 (s, 1H), 11.10 (br s, 1H), 11.40 (s, 1H). Mass spec: m/z 785.2 [M+H]⁺.

### Example 48 was Synthesized Following Scheme 47

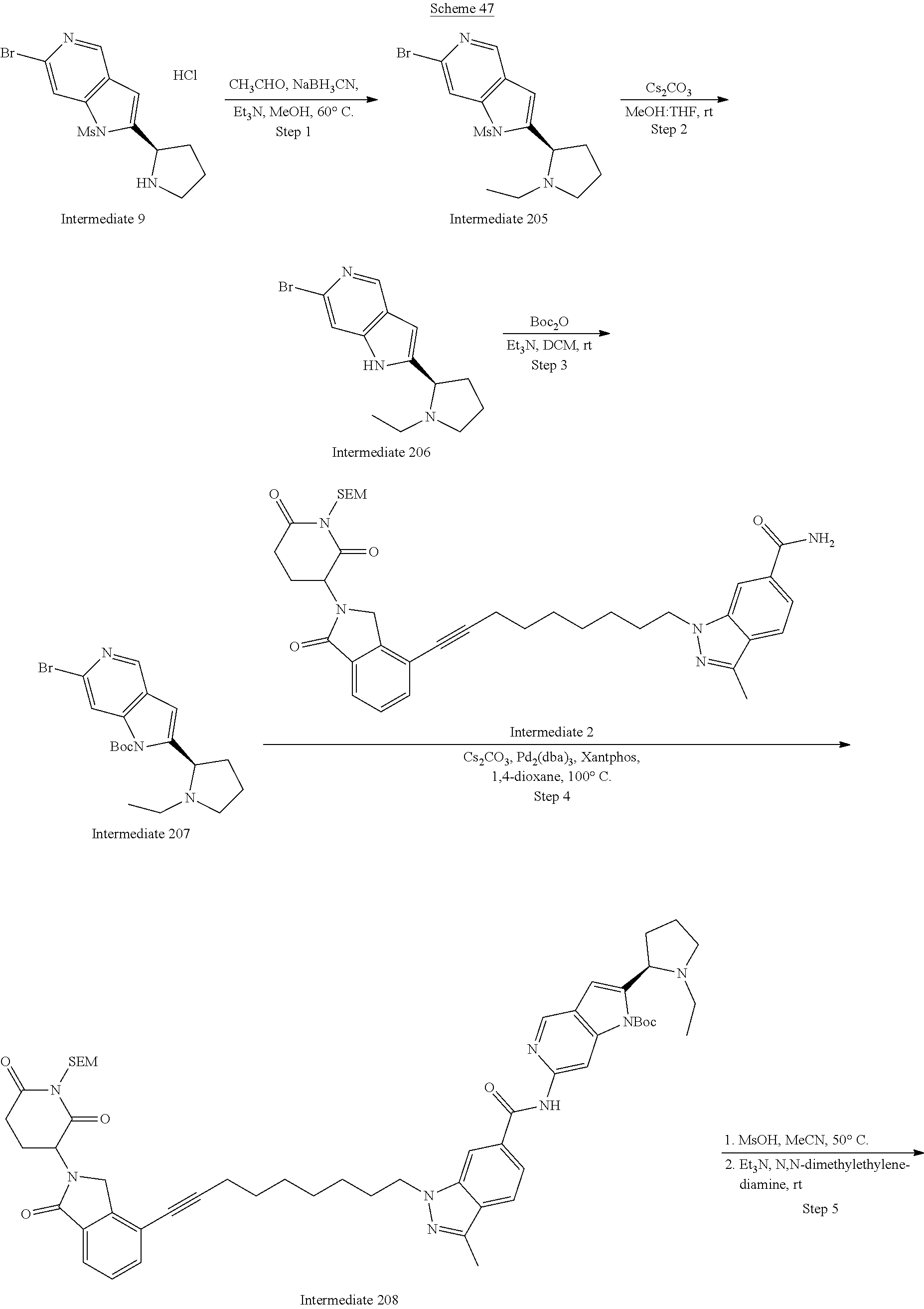

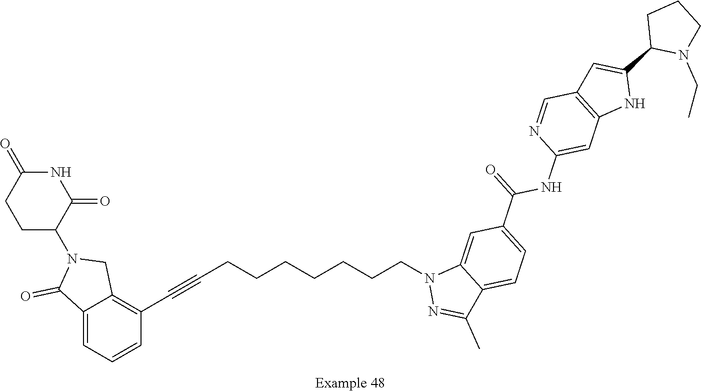 [SMILES: CCN1CCC[C@@H]1c1cc2cnc(NC(=O)c3ccc4c(C)nn(CCCCCCCC#Cc5cccc6c5CN(C5CCC(=O)NC5=O)C6=O)c4c3)cc2[nH]1]

### Step 1

### Intermediate 205: (R)-6-bromo-2-(1-ethylpyrrolidin-2-yl)-1-(methylsulfonyl)-1H-pyrrolo [3,2-c]pyridine

To a cooled (0° C.) solution of (R)-6-bromo-1-(methylsulfonyl)-2-(pyrrolidin-2-yl)-1H-pyrrolo[3,2-c]pyridine hydrochloride (Intermediate 9, 10.0 g, 26.27 mmol) in methanol (50 mL) was added triethylamine (29.4 mL, 210.1 mmol) and acetaldehyde (4.21 mL, 78.80 mmol) followed by sodium cyanoborohydride (3.88 g, 52.53 mmol). The reaction mixture was then heated at 60° C. for 16 h. The reaction mixture was diluted with water (200 mL) and extracted with ethyl acetate (3×200 mL). The combined organic layers were dried over anhydrous Na₂SO₄, filtered, and concentrated in vacuo. The crude material was purified by combi-flash chromatography, by eluting with 60% ethyl acetate in heptane, to afford (R)-6-bromo-2-(1-ethylpyrrolidin-2-yl)-1-(methylsulfonyl)-1H-pyrrolo[3,2-c]pyridine (Intermediate 205, 5.6 g, 57%) as a yellow solid. ¹H NMR (400 MHz, DMSO-d₆) δ ppm 0.97-1.17 (m, 3H), 1.64-1.82 (m, 3H), 2.20-2.38 (m, 3H), 2.72-2.84 (m, 1H), 3.22-3.31 (m, 1H), 3.55 (s, 3H), 3.95-4.04 (m, 1H), 6.92 (s, 1H), 7.96 (s, 1H), 8.67 (s, 1H). Mass spec m/z 373.1 [M+H+2]⁺.

### Step 2

### Intermediate 206: (R)-6-bromo-2-(1-ethylpyrrolidin-2-yl)-1H-pyrrolo [3,2-c]pyridine

To a cooled (0° C.) solution of (R)-6-bromo-2-(1-ethylpyrrolidin-2-yl)-1-(methylsulfonyl)-1H-pyrrolo[3,2-c]pyridine (Intermediate 205, 5.60 g, 15.04 mmol) in tetrahydrofuran (25 mL) and methanol (25 mL) was added cesium carbonate (9.81 g, 30.08 mmol). The reaction mixture was stirred at room temperature for 4 h. The reaction mixture was diluted with water (200 mL) and extracted with ethyl acetate (3×200 mL). The combined organic layers were dried over anhydrous Na₂SO₄, filtered, and concentrated in vacuo to afford crude (R)-6-bromo-2-(1-ethylpyrrolidin-2-yl)-1H-pyrrolo[3,2-c]pyridine (Intermediate 206, 4.1 g, 93%) as an off-white solid which was used for the next step without further purification. ¹H NMR (400 MHz, DMSO-d₆) δ ppm 0.91-0.97 (m, 3H), 1.69-1.88 (m, 3H), 2.06-2.20 (m, 3H), 2.49-2.55 (m, 1H), 3.17-3.21 (m, 1H), 3.44-3.48 (m, 1H), 6.40 (s, 1H), 7.40 (s, 1H), 8.44 (s, 1H), 11.48 (br s, 1H). Mass spec m/z 295.9 [M+H+2]⁺.

### Step 3

### Intermediate 207: tert-butyl-(R)-6-bromo-2-(1-ethylpyrrolidin-2-yl)-1H-pyrrolo[3,2-c]pyridine-1-carboxylate

To a solution of (R)-6-bromo-2-(1-ethylpyrrolidin-2-yl)-1H-pyrrolo[3,2-c]pyridine (Intermediate 206, 4.0 g, 14 mmol) in dichloromethane (40 mL) was added triethylamine (5.7 mL, 41 mmol) followed by di-tert-butyl dicarbonate (6.0 g, 27 mmol) and 4-dimethylaminopyridine (0.34 g, 2.7 mmol) and the reaction mixture was stirred at room temperature for 3 h. The reaction mixture was diluted with water (500 mL) and extracted with DCM (3×500 mL). The combined organic layers were dried over anhydrous Na₂SO₄, filtered, and concentrated in vacuo. The crude material was purified by combi-flash chromatography, by eluting with 45% ethyl acetate in heptane, to afford tert-butyl (R)-6-bromo-2-(1-ethylpyrrolidin-2-yl)-1H-pyrrolo[3,2-c]pyridine-1-carboxylate which was further purified by SFC chiral purification (Column: Chiralpak IK (250*30 mm) 5 um. Isocratic elution with 25% Mobile Phase (A): CO₂ and mobile Phase (B): MeCN+isopropyl alcohol with 0.1% isopropylamine) to afford tert-butyl-(R)-6-bromo-2-(1-ethylpyrrolidin-2-yl)-1H-pyrrolo[3,2-c]pyridine-1-carboxylate (Intermediate 207, 2.2 g, 41%) as an off white solid. ¹H NMR (400 MHz, DMSO-d₆) δ ppm 1.06 (t, J=7.13 Hz, 3H), 1.65 (s, 9H), 1.70-1.80 (m, 2H), 2.24-2.38 (m, 4H), 2.72-2.82 (m, 1H), 3.22-3.25 (m, 1H), 4.06-4.10 (m, 1H), 6.83 (s, 1H), 8.05 (s, 1H), 8.61 (s, 1H). Mass spec m/z 395.8 [M+H]⁺.

### Step 4

### Intermediate 208: tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((R)-1-ethylpyrrolidin-2-yl)-1H-pyrrolo[3,2-c]pyridine-1-carboxylate

To a solution of 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 2, 0.7 g, 1 mmol) and tert-butyl (R)-6-bromo-2-(1-ethylpyrrolidin-2-yl)-1H-pyrrolo[3,2-c]pyridine-1-carboxylate (Intermediate 207, 0.50 g, 1 mmol) in 1,4-dioxane (10 mL) was added cesium carbonate (0.68 g, 4 mmol). The reaction mixture was purged with argon gas for 15 min then Xantphos (0.2 g, 0.4 mmol) and Pd₂(dba) 3 (0.2 g, 0.2 mmol) were added. The reaction mixture was purged with argon gas for a further 10 min and then heated at 100° C. for 2 h. The reaction mixture was filtered through a celite bed and the filter pad was washed with ethyl acetate (50 mL). The filtrate was concentrated in vacuo and the crude material purified by combi-flash chromatography, by eluting with 90% ethyl acetate in heptane, to afford tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((R)-1-ethylpyrrolidin-2-yl)-1H-pyrrolo[3,2-c]pyridine-1-carboxylate (Intermediate 208, 0.42 g, 30%) as a yellow solid. ¹H NMR (400 MHz, DMSO-d₆) δ ppm −0.06 (s, 9H), 0.74-0.88 (m, 2H), 1.08 (t, J=7.02 Hz, 3H), 1.25-1.44 (m, 6H), 1.52-1.66 (m, 5H), 1.69 (s, 9H), 1.73-1.75 (m, 2H), 1.85-1.92 (m, 2H), 2.02-2.08 (m, 1H), 2.20-2.36 (m, 2H), 2.38 (s, 3H), 2.41-2.45 (m, 2H), 2.74-2.84 (m, 2H), 3.00-3.09 (m, 1H), 3.23-3.26 (m, 1H), 3.45-3.55 (m, 2H), 4.09-4.13 (m, 1H), 4.21-4.26 (m, 1H), 4.38-4.45 (m, 3H), 4.99-5.07 (m, 2H), 5.24-5.28 (m, 1H), 6.79 (s, 1H), 7.47-7.51 (m, 1H), 7.59-7.62 (m, 1H), 7.69-7.74 (m, 2H), 7.77-7.81 (m, 1H), 8.42 (s, 1H), 8.60 (s, 1H), 8.97 (s, 1H), 10.73 (s, 1H). Mass spec m/z 983.2 [M+H]⁺.

### Step 5

### Example 48: 1-(9-(2-(2,6-dioxopiperidin-3-yl)-1-oxoisoindolin-4-yl)non-8-yn-1-yl)-N-(2-((R)-1-ethylpyrrolidin-2-yl)-1H-pyrrolo[3,2-c]pyridin-6-yl)-3-methyl-1H-indazole-6-carboxamide

To a solution of tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((R)-1-ethylpyrrolidin-2-yl)-1H-pyrrolo[3,2-c]pyridine-1-carboxylate (Intermediate 208, 0.42 g, 0.42 mmol) in acetonitrile (8 mL) was added methanesulfonic acid (0.42 mL, 6.41 mmol) and the reaction mixture was heated at 50° C. for 2 h. The reaction mixture was cooled to room temperature then N,N′-dimethylethylenediamine (0.31 mL, 2.56 mmol) followed by triethylamine (1.20 mL, 8.54 mmol) were added and stirred at room temperature for 3 h. The reaction mixture was concentrated in vacuo and then water (20 mL) added. The resultant precipitate was collected by filtration and dried in vacuo to afford crude compound. The crude material was purified by preparative HPLC (Method A) to afford 1-(9-(2-(2,6-dioxopiperidin-3-yl)-1-oxoisoindolin-4-yl)non-8-yn-1-yl)-N-(2-((R)-1-ethylpyrrolidin-2-yl)-1H-pyrrolo[3,2-c]pyridin-6-yl)-3-methyl-1H-indazole-6-carboxamide (Example 48, 0.08 g, 27%) as an off white solid. ¹H NMR (400 MHz, DMSO-d₆) δ ppm 0.98 (t, J=7.25 Hz, 3H), 1.26-1.45 (m, 6H), 1.51-1.58 (m, 2H), 1.79-1.92 (m, 5H), 1.97-2.04 (m, 1H), 2.11-2.24 (m, 3H), 2.40 (s, 3H), 2.42-2.48 (m, 2H), 2.56-2.68 (m, 3H), 2.85-2.94 (m, 1H), 3.22-3.27 (m, 1H), 3.46-3.50 (m, 1H), 4.27-4.31 (m, 1H), 4.38-4.44 (m, 3H), 5.10-5.15 (m, 1H), 6.39 (s, 1H), 7.47-7.52 (m, 1H), 7.58-7.60 (m, 1H), 7.68-7.70 (m, 1H), 7.72-7.75 (m, 1H), 7.76-7.80 (m, 1H), 8.25 (s, 1H), 8.43 (s, 1H), 8.51 (s, 1H), 10.51 (s, 1H), 11.00 (s, 1H), 11.34 (s, 1H). Mass spec m/z 753.2 [M+H]⁺.

### Example 49 was Synthesized Following Scheme 48

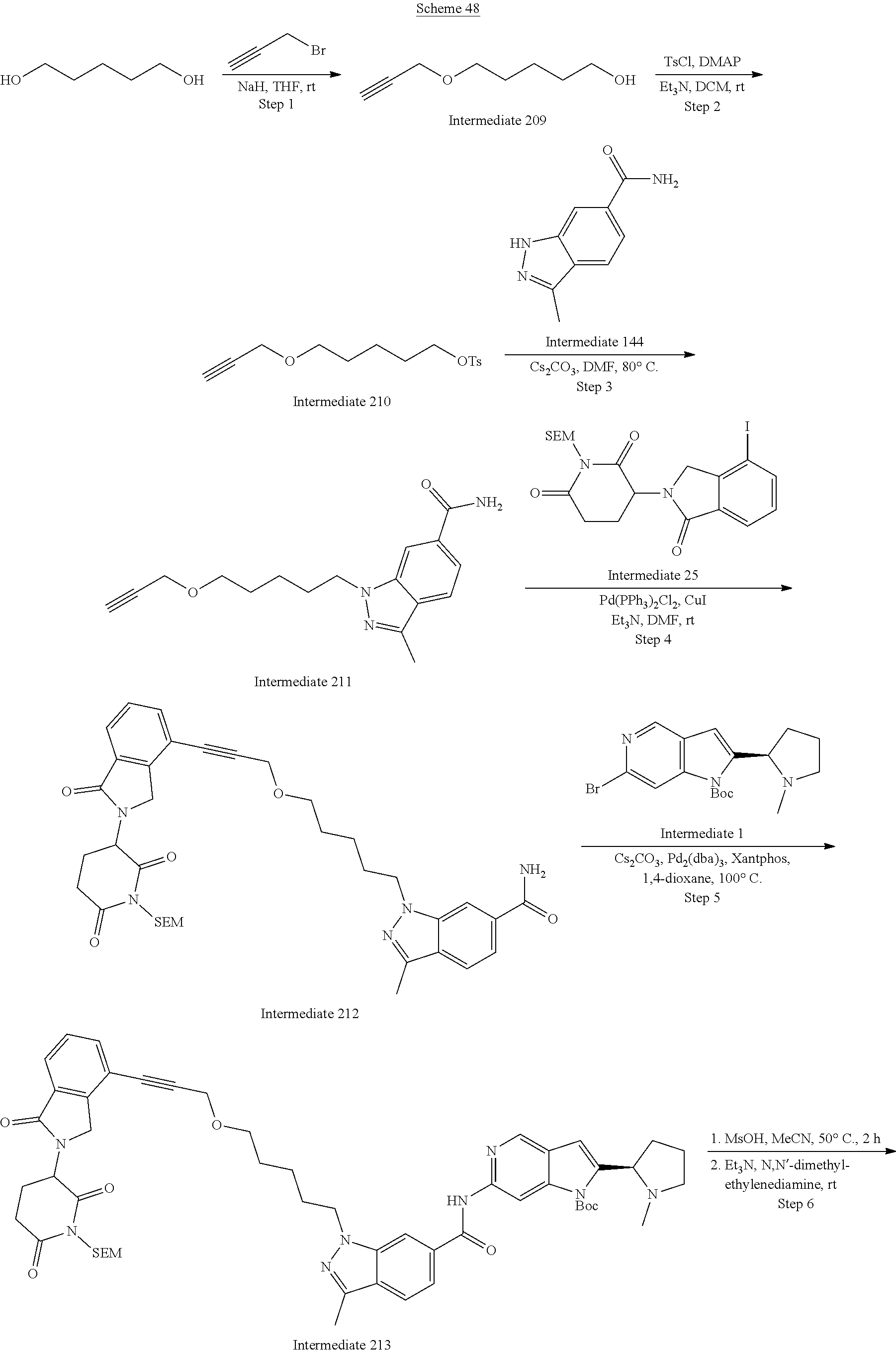

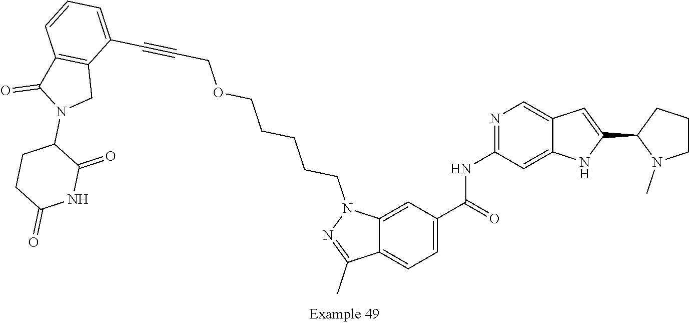 [SMILES: Cc1nn(CCCCCOCC#Cc2cccc3c2CN(C2CCC(=O)NC2=O)C3=O)c2cc(C(=O)Nc3cc4[nH]c([C@H]5CCCN5C)cc4cn3)ccc12]

### Step 1

### Intermediate 209: 5-(prop-2-yn-1-yloxy) pentan-1-ol

To a cooled (0° C.) solution of sodium hydride (0.80 g, 33.62 mmol) in tetrahydrofuran (40 mL) was added pentane-1,5-diol (CAS No: 111-29-5, 13.65 g, 131.13 mmol) and the reaction was stirred for 30 min at 0° C. prior to the addition of 3-bromoprop-1-yne (CAS No: 106-96-7, 4.00 g, 33.6 mmol). The reaction mixture was stirred at room temperature for 16 h. The reaction mixture was poured into ice cold water (150 mL) and extracted with DCM (2×200 mL). The combined organic layers were dried over anhydrous Na₂SO₄, filtered, and concentrated in vacuo. The crude material was purified by combi-flash chromatography, by eluting with 70% ethyl acetate in heptane, to afford 5-(prop-2-yn-1-yloxy) pentan-1-ol (Intermediate 209, 3.5 g, 74%) as a yellow liquid. ¹H NMR (400 MHz, CDCl₃) δ ppm 1.38-1.46 (m, 3H), 1.54-1.66 (m, 4H), 2.39 (s, 1H), 3.51 (t, J=6.43 Hz, 2H), 3.63 (t, J=6.43 Hz, 2H), 4.11 (s, 2H).

### Step 2

### Intermediate 210: 5-(prop-2-yn-1-yloxy) pentyl 4-methylbenzenesulfonate

To a cooled (0° C.) solution of 5-(prop-2-yn-1-yloxy)pentan-1-ol (Intermediate 209, 1.60 g, 11.3 mmol) in dichloromethane (80 mL) was added triethylamine (2.38 mL, 16.9 mmol) followed by 4-dimethylaminopyridine (0.14 g, 1.13 mmol) and the reaction was stirred at 0° C. for 15 min prior to the addition of p-toluenesulfonyl chloride (2.63 g, 13.5 mmol). The reaction mixture was stirred at room temperature for 16 h. The reaction mixture was diluted with 1N HCl (100 mL) and extracted with dichloromethane (2×100 mL). The combined organic layers were washed with NaHCO₃ (2×100 mL) and brine solution (100 mL), dried over anhydrous Na₂SO₄, and concentrated in vacuo to afford 5-(prop-2-yn-1-yloxy) pentyl 4-methylbenzenesulfonate (Intermediate 210, 3.25 g, 97%) as a yellow oil which was used for the next step without further purification. ¹H NMR (400 MHz, DMSO-d₆) δ ppm 1.21-1.29 (m, 2H), 1.38-1.45 (m, 2H), 1.53-1.60 (m, 2H), 2.42 (s, 3H), 3.33-3.39 (m, 2H), 3.39 (s, 1H), 4.01 (t, J=6.36 Hz, 2H), 4.07 (s, 2H), 7.48 (d, J=8.40 Hz, 2H), 7.78 (d, J=8.11 Hz, 2H).

### Step 3

### Intermediate 211: 3-methyl-1-(5-(prop-2-yn-1-yloxy) pentyl)-1H-indazole-6-carboxamide

To solution of 3-methyl-1H-indazole-6-carboxamide (Intermediate 144, 1.6 g, 9.1 mmol) in dimethylformamide (30 mL) was added cesium carbonate (8.9 g, 27 mmol) and 5-(prop-2-yn-1-yloxy)pentyl-4-methylbenzenesulfonate (Intermediate 210, 3.2 g, 11 mmol) and the reaction mixture was heated at 80° C. for 4 h. The reaction mixture was diluted with water (100 mL) and extracted with ethyl acetate (3×60 mL). The combined organic layers were washed with brine solution (2×50 mL), dried over anhydrous Na₂SO₄, and concentrated in vacuo to obtain crude compound as a mixture of regioisomers. The regioisomers were separated by combi-flash chromatography, by eluting with 60% ethyl acetate in heptane, to afford desired regioisomer 3-methyl-1-(5-(prop-2-yn-1-yloxy)pentyl)-1H-indazole-6-carboxamide (Intermediate 211, 1.40 g, 51%) as a white solid. ¹H NMR (400 MHz, DMSO-d₆) δ ppm 1.24-1.32 (m, 2H), 1.49-1.56 (m, 2H), 1.79-1.86 (m, 2H), 2.49 (s, 3H), 3.36-3.39 (m, 2H), 3.40 (s, 1H), 4.06 (s, 2H), 4.33 (t, J=7.02 Hz, 2H), 7.41 (br s, 1H), 7.59 (d, J=8.33 Hz, 1H), 7.73 (d, J=8.33 Hz, 1H), 8.02 (br s, 1H), 8.12 (s, 1H). Mass spec m/z 299.9 [M+H]⁺.

### Step 4

### Intermediate 212: 1-(5-((3-(2-(2,6-dioxo-1-((2-(trimethylsilyl)ethoxy)methyl)piperidin-3-yl)-1-oxoisoindolin-4-yl)prop-2-yn-1-yl) oxy) pentyl)-3-methyl-1H-indazole-6-carboxamide

To solution of 3-methyl-1-(5-(prop-2-yn-1-yloxy)pentyl)-1H-indazole-6-carboxamide (Intermediate 211, 1.10 g, 3.67 mmol) and 3-(4-iodo-1-oxoisoindolin-2-yl)-1-((2-(trimethylsilyl)ethoxy)methyl)piperidine-2,6-dione (Intermediate 25, 2.02 g, 4.04 mmol) in dimethylformamide (8 mL) was added triethylamine (18 mL, 129 mmol). The reaction mixture was purged with argon gas for 10 min then PdCl₂(PPh₃)₂ (0.26 g, 0.36 mmol) and copper (I) iodide (0.07 g, 0.36 mmol) were added at room temperature. The reaction mixture was purged with argon gas for 10 min and stirred at room temperature for 2 h. The reaction mixture was diluted with water (60 mL) and extracted with ethyl acetate (3×50 mL). The combined organic layers were washed with brine solution (2×50 mL), dried over anhydrous Na₂SO₄, and concentrated in vacuo. The crude material was purified by combi-flash chromatography, by eluting with 2% MeOH in DCM, to afford 1-(5-((3-(2-(2,6-dioxo-1-((2-(trimethylsilyl) ethoxy)methyl)piperidin-3-yl)-1-oxoisoindolin-4-yl)prop-2-yn-1-yl)oxy)pentyl)-3-methyl-1H-indazole-6-carboxamide (Intermediate 212, 1.30 g, 53%) as a white solid. ¹H NMR (400 MHz, DMSO-d₆) δ ppm −0.04 (s, 9H), 0.78-0.86 (m, 2H), 1.28-1.35 (m, 3H), 1.54-1.61 (m, 2H), 1.81-1.88 (m, 2H), 2.01-2.08 (m, 1H), 2.39-2.44 (m, 1H), 2.67-2.80 (m, 3H), 3.01-3.11 (m, 1H), 3.48-3.54 (m, 4H), 4.27-4.40 (m, 5H), 4.47-4.51 (m, 1H), 5.00-5.08 (m, 2H), 5.24-5.29 (m, 1H), 7.40 (br s, 1H), 7.54-7.60 (m, 2H), 7.69-7.73 (m, 2H), 7.77 (d, J=7.89 Hz, 1H), 8.01 (br s, 1H), 8.12 (s, 1H). Mass spec m/z 672.0 [M+H]⁺.

### Step 5

### Intermediate 213: tert-butyl 6-(1-(5-((3-(2-(2,6-dioxo-1-((2-(trimethylsilyl)ethoxy)methyl)piperidin-3-yl)-1-oxoisoindolin-4-yl)prop-2-yn-1-yl)oxy)pentyl)-3-methyl-1H-indazole-6-carboxamido)-2-((R)-1-methylpyrrolidin-2-yl)-1H-pyrrolo[3,2-c]pyridine-1-carboxylate

To a solution of 1-(5-((3-(2-(2,6-dioxo-1-((2-(trimethylsilyl) ethoxy)methyl) piperidin-3-yl)-1-oxoisoindolin-4-yl)prop-2-yn-1-yl) oxy) pentyl)-3-methyl-1H-indazole-6-carboxamide (Intermediate 212, 0.60 g, 0.89 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 1, 0.40 g, 1.07 mmol) followed by cesium carbonate (0.87 g, 2.67 mmol). The reaction mixture was purged with argon gas for 15 min then Pd₂(dba)₃ (0.12 g, 0.14 mmol) and Xantphos (0.16 g, 0.26 mmol) were added and the reaction mixture was purged with argon gas for 10 min. The reaction mixture was heated at 100° C. for 2 h then diluted with water (40 mL) and extracted with ethyl acetate (3×50 mL). The combined organic layers were washed with brine solution (2×50 mL), dried over anhydrous Na₂SO₄, and concentrated in vacuo. The crude material was purified by combi-flash chromatography, by eluting with 2% MeOH in DCM, to afford tert-butyl 6-(1-(5-((3-(2-(2,6-dioxo-1-((2-(trimethylsilyl)ethoxy)methyl)piperidin-3-yl)-1-oxoisoindolin-4-yl)prop-2-yn-1-yl)oxy)pentyl)-3-methyl-1H-indazole-6-carboxamido)-2-((R)-1-methylpyrrolidin-2-yl)-1H-pyrrolo[3,2-c]pyridine-1-carboxylate (Intermediate 213, 0.40 g, 46%) as a yellow solid. ¹H NMR (400 MHz, DMSO-d₆) δ ppm −0.05 (s, 9H), 0.76-0.85 (m, 2H), 1.32-1.39 (m, 2H), 1.56-1.65 (m, 3H), 1.70 (s, 9H), 1.73-1.76 (m, 3H), 1.86-1.92 (m, 2H), 2.01-2.08 (m, 2H), 2.31-2.33 (m, 1H), 2.36 (s, 3H), 2.42 (s, 3H), 2.74-2.78 (m, 1H), 3.00-3.14 (m, 2H), 3.47-3.55 (m, 4H), 3.91-3.94 (m, 1H), 4.26-4.30 (m, 1H), 4.38-4.51 (m, 5H), 4.98-5.06 (m, 2H), 5.24-5.28 (m, 1H), 6.76 (s, 1H), 7.52-7.55 (m, 1H), 7.67-7.79 (m, 4H), 8.43 (s, 1H), 8.60 (s, 1H), 8.97 (s, 1H), 10.74 (s, 1H). Mass spec m/z 971.3 [M+H]⁺.

### Step 6

### Example 49: 1-(5-((3-(2-(2,6-dioxopiperidin-3-yl)-1-oxoisoindolin-4-yl)prop-2-yn-1-yl)oxy)pentyl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(5-((3-(2-(2,6-dioxo-1-((2-(trimethylsilyl)ethoxy)methyl)piperidin-3-yl)-1-oxoisoindolin-4-yl)prop-2-yn-1-yl)oxy)pentyl)-3-methyl-1H-indazole-6-carboxamido)-2-((R)-1-methylpyrrolidin-2-yl)-1H-pyrrolo[3,2-c]pyridine-1-carboxylate (Intermediate 213, 0.38 g, 0.39 mmol) in acetonitrile (6 mL) was added methanesulfonic acid (0.38 mL, 5.86 mmol) and the reaction mixture was heated to 50° C. for 2 h. The reaction mixture was cooled to room temperature, then N,N′-dimethylethylenediamine (0.28 mL, 2.34 mmol) followed by triethylamine (0.82 mL, 5.87 mmol) were added and the mixture stirred at room temperature for 2 h. The reaction mixture was concentrated in vacuo and the resultant solid was washed with ether (2×50 mL) and DCM/Pentanes (2×40 mL) and dried in vacuo. The crude material was purified by preparative HPLC (Method A) to afford 1-(5-((3-(2-(2,6-dioxopiperidin-3-yl)-1-oxoisoindolin-4-yl)prop-2-yn-1-yl)oxy)pentyl)-3-methyl-N-(2-((R)-1-methylpyrrolidin-2-yl)-1H-pyrrolo[3,2-c]pyridin-6-yl)-1H-indazole-6-carboxamide (Example 49, 0.067 g, 23%) as an off white solid. ¹H NMR (400 MHz, DMSO-d₆) δ ppm 1.32-1.40 (m, 2H), 1.57-1.64 (m, 2H), 1.79-2.01 (m, 6H), 2.13-2.15 (m, 1H), 2.17 (s, 3H), 2.24-2.30 (m, 1H), 2.47 (s, 3H), 2.58-2.60 (m, 1H), 2.85-2.94 (m, 1H), 3.12-3.17 (m, 1H), 3.27-3.29 (m, 1H), 3.33-3.35 (m, 1H), 3.50-3.53 (m, 2H), 4.30-4.49 (m, 6H), 5.11-5.15 (m, 1H), 6.40 (s, 1H), 7.51-7.55 (m, 1H), 7.67-7.79 (m, 4H), 8.25 (s, 1H), 8.44 (s, 1H), 8.51 (s, 1H), 10.52 (s, 1H), 11.00 (br s, 1H), 11.39 (s, 1H). Mass spec: m/z: 741.3 [M+H]⁺.

### Example 50 was Synthesized Following Scheme 49

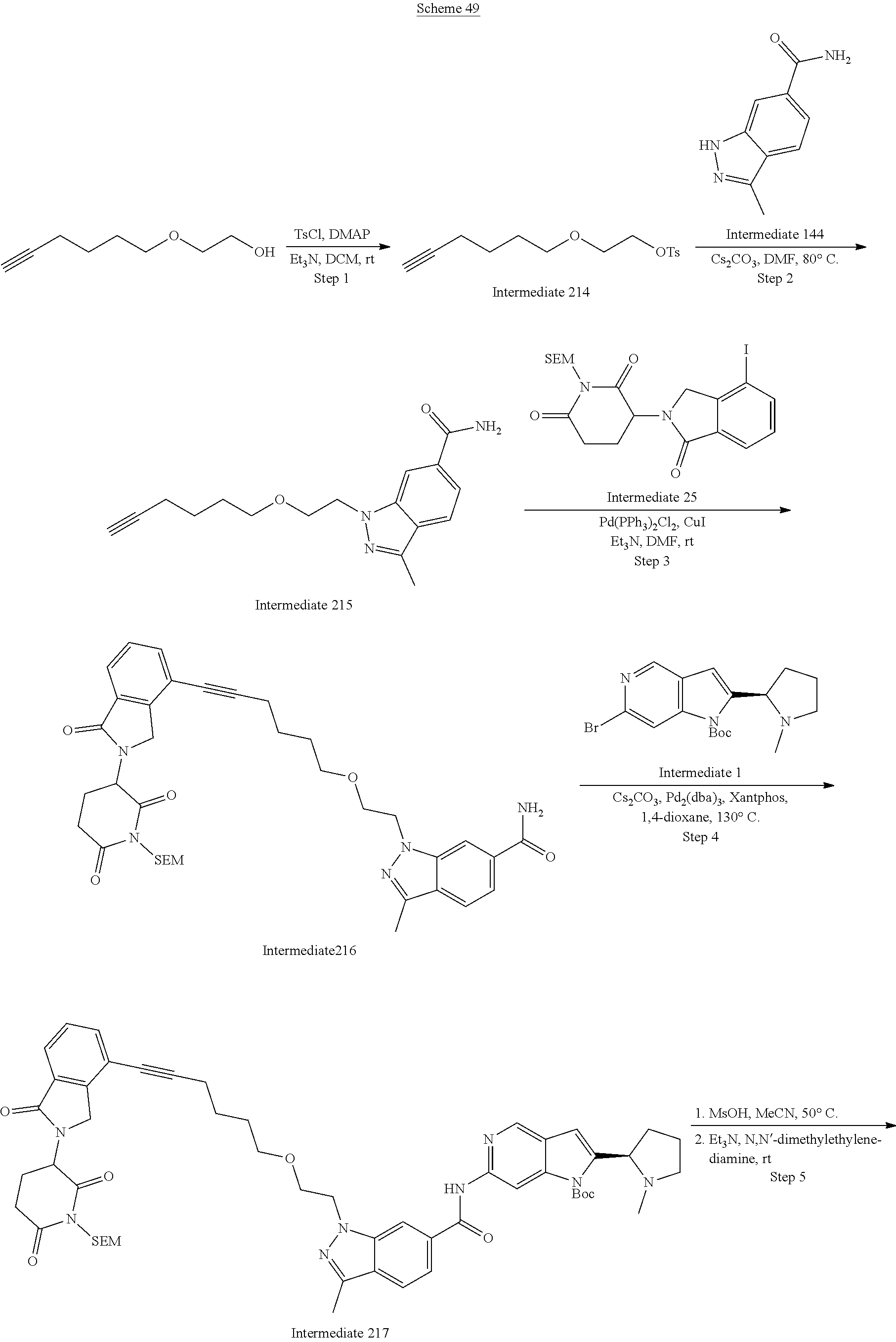

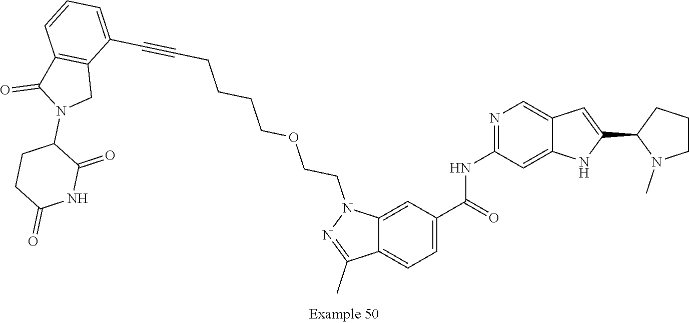 [SMILES: Cc1nn(CCOCCCCC#Cc2cccc3c2CN(C2CCC(=O)NC2=O)C3=O)c2cc(C(=O)Nc3cc4[nH]c([C@H]5CCCN5C)cc4cn3)ccc12]

### Step 1

### Intermediate 214: 2-(hex-5-yn-1-yloxy) ethyl 4-methylbenzenesulfonate

To a cooled (0° C.) solution of 2-(hex-5-yn-1-yloxy)ethan-1-ol (CAS No: 252948-15-5, 3.00 g, 21.09 mmol) in dichloromethane (30 mL) was added sequentially triethylamine (4.43 mL, 31.64 mmol), 4-dimethylaminopyridine (0.52 g, 4.22 mmol) and p-toluenesulfonyl chloride (6.35 g, 31.64 mmol). The reaction mixture was stirred at room temperature for 16 h then diluted with water (150 mL) and extracted with dichloromethane (3×150 mL). The combined organic layers were separated, dried over anhydrous Na₂SO₄ and concentrated in vacuo. The crude material was purified by combi-flash chromatography, by eluting with 40% ethyl acetate in heptane, to afford 2-(hex-5-yn-1-yloxy) ethyl 4-methylbenzenesulfonate (Intermediate 214, 3.4 g, 54%) as a colourless oil. ¹H NMR (400 MHz, DMSO-d₆) δ ppm 1.36-1.43 (m, 2H), 1.45-1.52 (m, 2H), 2.11-2.16 (m, 2H), 2.42 (s, 3H), 2.73-2.75 (m, 1H), 3.29-3.34 (m, 2H), 3.49-3.54 (m, 2H), 4.10-4.14 (m, 2H), 7.48 (d, J=7.88 Hz, 2H), 7.78 (d, J=7.88 Hz, 2H).

### Step 2

### Intermediate 215: 1-(2-(hex-5-yn-1-yloxy) ethyl)-3-methyl-1H-indazole-6-carboxamide

To solution of 2-(hex-5-yn-1-yloxy) ethyl 4-methylbenzenesulfonate (Intermediate 214, 2.03 g, 6.84 mmol) in dimethylformamide (10 mL) was added 3-methyl-1H-indazole-6-carboxamide (Intermediate 144, 1 g, 5.71 mmol) and cesium carbonate (5.57 g, 17.12 mmol). The reaction mixture was heated at 80° C. for 16 h. The reaction mixture was diluted with water (50 mL) and extracted with ethyl acetate (3×100 mL). The combined organic layers were separated, dried over anhydrous Na₂SO₄, concentrated in vacuo to obtain crude compound as a mixture of regioisomers. The regioisomers were separated by combi-flash chromatography, by eluting with 80% ethyl acetate in heptane, to afford desired regioisomer 1-(2-(hex-5-yn-1-yloxy)ethyl)-3-methyl-1H-indazole-6-carboxamide (Intermediate 215, 0.8 g, 47%) as a white solid. ¹H NMR (400 MHz, DMSO-d₆) δ ppm 1.28-1.34 (m, 2H), 1.41-1.46 (m, 2H), 2.00-2.04 (m, 2H), 2.49 (s, 3H), 2.68 (s, 1H), 3.31-3.35 (m, 2H), 3.77 (t, J=5.48 Hz, 2H), 4.49 (t, J=5.48 Hz, 2H), 7.38 (br s, 1H), 7.59 (d, J=8.44 Hz, 1H), 7.72 (d, J=8.33 Hz, 1H), 8.00 (br s, 1H), 8.15 (s, 1H). Mass spec m/z 300.19 [M+H]⁺.

### Step 3

### Intermediate 216: 1-(2-((6-(2-(2,6-dioxo-1-((2-(trimethylsilyl) ethoxy) methyl) piperidin-3-yl)-1-oxoisoindolin-4-yl) hex-5-yn-1-yl) oxy) ethyl)-3-methyl-1H-indazole-6-carboxamide

To solution of 1-(2-(hex-5-yn-1-yloxy)ethyl)-3-methyl-1H-indazole-6-carboxamide (Intermediate 215, 0.80 g, 2.67 mmol) and 3-(4-iodo-1-oxoisoindolin-2-yl)-1-((2-(trimethylsilyl)ethoxy)methyl)piperidine-2,6-dione (Intermediate 25, 1.34 g, 2.67 mmol) in dimethylformamide (10 mL) was added triethylamine (13.8 mL, 98.86 mmol). The reaction mixture was purged with argon gas for 15 min then PdCl₂(PPh₃)₂ (0.19 g, 0.26 mmol) and copper (I) iodide (0.050 g, 0.26 mmol) were added. The reaction mixture was purged with argon gas for 5 min and stirred at room temperature for 3 h. The reaction mixture was poured into water (60 mL) and extracted with ethyl acetate (3×100 mL). The combined organic layers were separated, dried over anhydrous Na₂SO₄, and concentrated in vacuo. The crude material was purified by combi-flash chromatography, by eluting with 90% ethyl acetate in heptane, to afford 1-(2-((6-(2-(2,6-dioxo-1-((2-(trimethylsilyl)ethoxy)methyl)piperidin-3-yl)-1-oxoisoindolin-4-yl)hex-5-yn-1-yl)oxy)ethyl)-3-methyl-1H-indazole-6-carboxamide (Intermediate 216, 0.80 g, 45%) as an off white solid. Mass spec m/z 672.1 [M+H]⁺.

### Step 4

### Intermediate 217: tert-butyl 6-(1-(2-((6-(2-(2,6-dioxo-1-((2-(trimethylsilyl)ethoxy)methyl)piperidin-3-yl)-1-oxoisoindolin-4-yl)hex-5-yn-1-yl)oxy)ethyl)-3-methyl-1H-indazole-6-carboxamido)-2-((R)-1-methylpyrrolidin-2-yl)-1H-pyrrolo[3,2-c]pyridine-1-carboxylate

To a solution of 1-(2-((6-(2-(2,6-dioxo-1-((2-(trimethylsilyl)ethoxy)methyl)piperidin-3-yl)-1-oxoisoindolin-4-yl)hex-5-yn-1-yl)oxy)ethyl)-3-methyl-1H-indazole-6-carboxamide (Intermediate 216, 0.71 g, 1.05 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 1, 0.50 g, 1.31 mmol) followed by cesium carbonate (1.08 g, 3.17 mmol). The reaction mixture was purged with argon gas for 15 min then Pd₂(dba)₃ (0.18 g, 0.16 mmol) and Xantphos (0.19 g, 0.32 mmol) were added and the reaction mixture was purged with argon gas for 5 min. The reaction mixture was heated at 130° C. for 2 h. The reaction mixture was concentrated in vacuo and the crude material was purified by combi-flash chromatography, by eluting with 90% ethyl acetate in heptane, to afford tert-butyl 6-(1-(2-((6-(2-(2,6-dioxo-1-((2-(trimethylsilyl)ethoxy)methyl)piperidin-3-yl)-1-oxoisoindolin-4-yl)hex-5-yn-1-yl)oxy)ethyl)-3-methyl-1H-indazole-6-carboxamido)-2-((R)-1-methylpyrrolidin-2-yl)-1H-pyrrolo[3,2-c]pyridine-1-carboxylate (Intermediate 217, 0.70 g, 70%) as an off white solid. ¹H NMR (400 MHz, DMSO-d₆) δ ppm −0.06 (s, 9H), 0.77-0.86 (m, 2H), 1.43-1.55 (m, 5H), 1.61-1.64 (m, 2H), 1.69 (s, 9H), 1.74-1.76 (m, 2H), 1.98-2.06 (m, 2H), 2.31 (s, 3H), 2.36 (s, 3H), 2.36-2.41 (m, 2H), 2.74-2.79 (m, 1H), 3.00-3.13 (m, 2H), 3.39-3.42 (m, 2H), 3.48-3.52 (m, 2H), 3.80-3.83 (m, 2H), 3.90-3.96 (m, 1H), 4.18-4.23 (m, 1H), 4.36-4.40 (m, 1H), 4.58 (s, 2H), 4.98-5.07 (m, 2H), 5.22-5.27 (m, 1H), 6.75 (s, 1H), 7.44-7.48 (m, 1H), 7.54 (d, J=7.60 Hz, 1H), 7.67 (d, J=7.20 Hz, 1H), 7.71-7.77 (m, 2H), 8.47 (s, 1H), 8.59 (s, 1H), 8.96 (s, 1H), 10.68 (s, 1H). Mass spec m/z 971.3 [M+H]⁺.

### Step 5

### Example 50: 1-(2-((6-(2-(2,6-dioxopiperidin-3-yl)-1-oxoisoindolin-4-yl)hex-5-yn-1-yl)oxy)ethyl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(2-((6-(2-(2,6-dioxo-1-((2-(trimethylsilyl)ethoxy)methyl)piperidin-3-yl)-1-oxoisoindolin-4-yl)hex-5-yn-1-yl)oxy)ethyl)-3-methyl-1H-indazole-6-carboxamido)-2-((R)-1-methylpyrrolidin-2-yl)-1H-pyrrolo[3,2-c]pyridine-1-carboxylate (Intermediate 217, 0.70 g, 0.72 mmol) in acetonitrile (15 mL) was added methanesulfonic acid (1.09 g, 10.81 mmol) and the reaction mixture was heated to 50° C. for 2 h. The reaction mixture was cooled to room temperature, then N,N′-dimethylethylenediamine (0.45 g, 4.32 mmol) followed by triethylamine (1.15 g, 10.81 mmol) were added and the mixture stirred at room temperature for 3 h. The reaction mixture was concentrated in vacuo and poured into water (20 mL). The resultant precipitate was collected by filtration and dried in vacuo. The crude material was purified by preparative HPLC (Method A) to afford 1-(2-((6-(2-(2,6-dioxopiperidin-3-yl)-1-oxoisoindolin-4-yl)hex-5-yn-1-yl)oxy)ethyl)-3-methyl-N-(2-((R)-1-methylpyrrolidin-2-yl)-1H-pyrrolo[3,2-c]pyridin-6-yl)-1H-indazole-6-carboxamide (Example 50, 0.15 g, 28%) as an off white solid. ¹H NMR (400 MHz, DMSO-d₆) δ ppm 1.41-1.50 (m, 2H), 1.50-1.59 (m, 2H), 1.76-2.02 (m, 4H), 2.13-2.15 (m, 1H), 2.17 (s, 3H), 2.23-2.34 (m, 3H), 2.47 (s, 3H), 2.55-2.61 (m, 2H), 2.84-2.95 (m, 1H), 3.12-3.18 (m, 1H), 3.22-3.29 (m, 1H), 3.41 (t, J=6.07 Hz, 2H), 3.83-3.85 (m, 2H), 4.23-4.30 (m, 1H), 4.36-4.42 (m, 1H), 4.58-4.60 (m, 2H), 5.09-5.14 (m, 1H), 6.40 (s, 1H), 7.45-7.49 (m, 1H), 7.54-7.58 (m, 1H), 7.67 (d, J=7.25 Hz, 1H), 7.71-7.77 (m, 2H), 8.24 (s, 1H), 8.47 (s, 1H), 8.51 (s, 1H), 10.47 (s, 1H), 11.00 (s, 1H), 11.37 (s, 1H). Mass spec m/z 741.2 [M+H]⁺.

### Example 51 was Synthesised Following Scheme 50

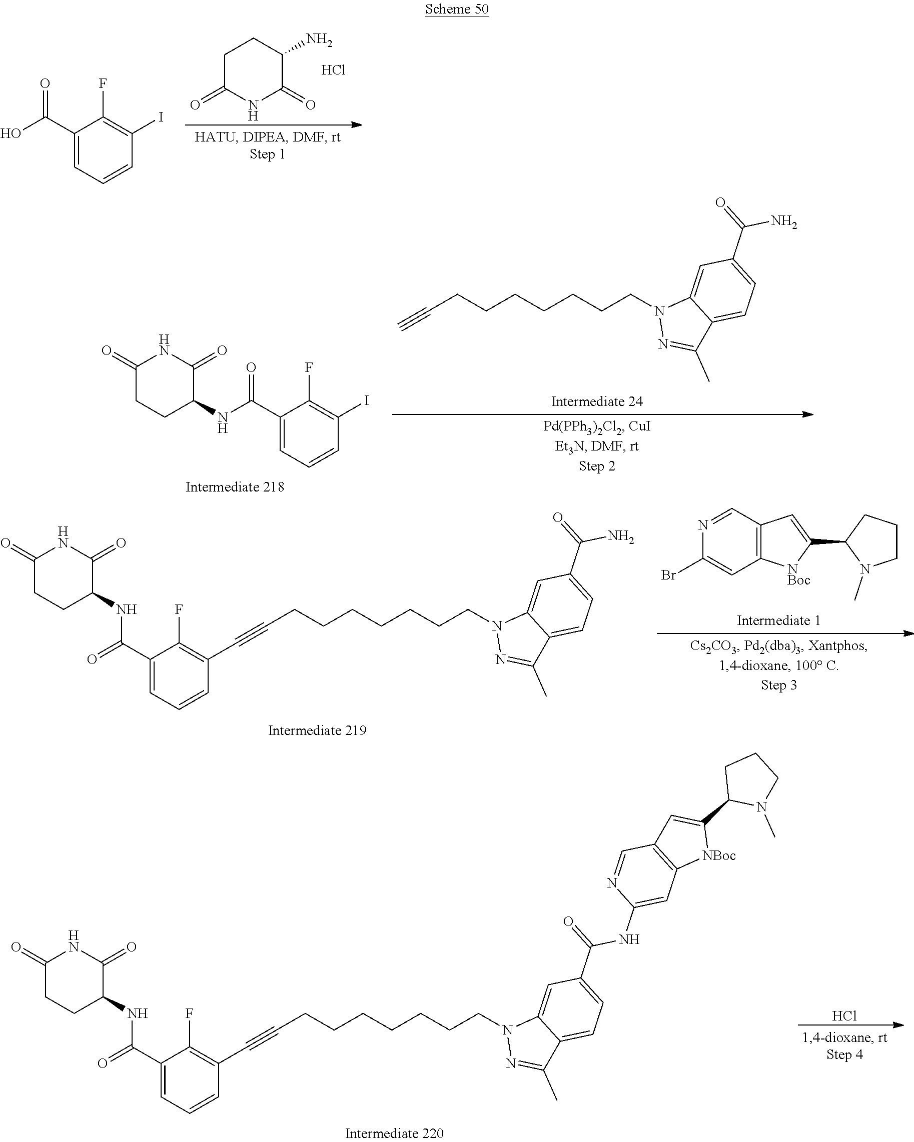

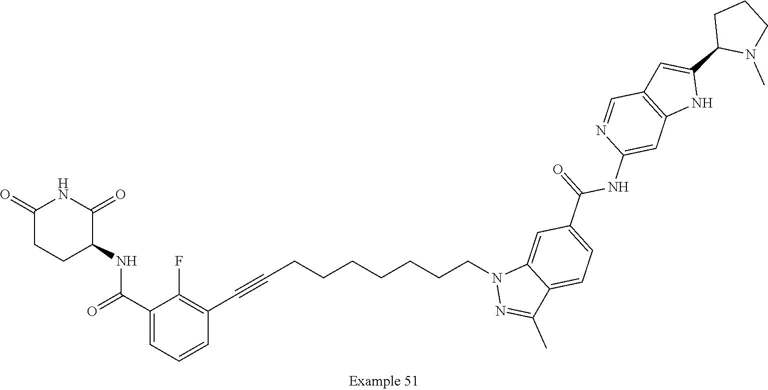 [SMILES: Cc1nn(CCCCCCCC#Cc2cccc(C(=O)N[C@H]3CCC(=O)NC3=O)c2F)c2cc(C(=O)Nc3cc4[nH]c([C@H]5CCCN5C)cc4cn3)ccc12]

### Step 1

### Intermediate 218: (S)—N-(2,6-dioxopiperidin-3-yl)-2-fluoro-3-iodobenzamide

To a solution of 2-fluoro-3-iodobenzoic acid (CAS No: 447464-03-1, 3.55 g, 13.36 mmol) in dimethylformamide (50 mL) was added HATU (7.29 g, 18.22 mmol) and DIPEA (3.20 g, 24.30 mmol) and the mixture was stirred for 10 min prior to the addition of (S)-3-aminopiperidine-2,6-dione hydrochloride (CAS No: 25181-50-4, 2.00 g, 12.15 mmol). The reaction mixture was stirred for 16 h then quenched by the addition of ice cold water (100 mL). The resultant precipitate was collected by filtration and dried in vacuo to afford (S)—N-(2,6-dioxopiperidin-3-yl)-2-fluoro-3-iodobenzamide (Intermediate 218, 3.7 g, 81%) as a white solid which was used for the next step without further purification. ¹H NMR (400 MHz, DMSO-d₆) δ ppm 2.01-2.14 (m, 2H), 2.52-2.58 (m, 1H), 2.74-2.85 (m, 1H), 4.73-4.80 (m, 1H), 7.11 (t, J=7.67 Hz, 1H), 7.61 (t, J=7.05 Hz, 1H), 7.98 (t, J=6.84 Hz, 1H), 8.72 (d, J=7.88 Hz, 1H), 10.86 (s, 1H). Mass spec m/z 376.9 [M+H]⁺.

### Step 2

### Intermediate 219: (S)-1-(9-(3-((2,6-dioxopiperidin-3-yl) carbamoyl)-2-fluorophenyl) non-8-yn-1-yl)-3-methyl-1H-indazole-6-carboxamide

To a solution of (S)—N-(2,6-dioxopiperidin-3-yl)-2-fluoro-3-iodobenzamide (Intermediate 218, 1.00 g, 2.65 mmol) and 3-methyl-1-non-8-ynyl-indazole-6-carboxamide (Intermediate 24, 0.79 g, 2.65 mmol) in dimethylformamide (30 mL) was added triethylamine (10.19 g, 95.71 mmol). The reaction mixture was purged with argon gas for 15 min then PdCl₂(PPh₃)₂ (0.19 g, 0.26 mmol) and copper (I) iodide (0.05 g, 0.26 mmol) were added. The reaction mixture was purged with argon gas for 5 min and stirred at room temperature for 2 h. The reaction mixture was diluted with water (100 mL) and extracted with ethyl acetate (3×150 mL). The combined organic layers were dried over anhydrous Na₂SO₄, filtered and concentrated in vacuo. The crude material was purified by combi-flash chromatography, by eluting with 90% ethyl acetate in heptane, to afford (S)-1-(9-(3-((2,6-dioxopiperidin-3-yl)carbamoyl)-2-fluorophenyl)non-8-yn-1-yl)-3-methyl-1H-indazole-6-carboxamide (Intermediate 219, 0.95 g, 65%) as a yellow solid. ¹H NMR (400 MHz, DMSO-d₆) δ ppm 1.25-1.44 (m, 8H), 1.47-1.56 (m, 2H), 1.78-1.87 (m, 2H), 1.99-2.12 (m, 2H), 2.22-2.25 (m, 1H), 2.53 (s, 3H), 2.73-2.83 (m, 1H), 4.31-4.37 (m, 2H), 4.73-4.79 (m, 1H), 7.25 (t, J=7.67 Hz, 1H), 7.41 (br s, 1H), 7.55-7.62 (m, 3H), 7.72 (d, J=8.33 Hz, 1H), 8.02 (br s, 1H), 8.12 (s, 1H), 8.68 (d, J=8.11 Hz, 1H), 10.86 (s, 1H). Mass spec m/z 546.1 [M+H]⁺.

### Step 3

### Intermediate 220: tert-butyl 6-(1-(9-(3-(((S)-2,6-dioxopiperidin-3-yl)carbamoyl)-2-fluorophenyl)non-8-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of (S)-1-(9-(3-((2,6-dioxopiperidin-3-yl) carbamoyl)-2-fluorophenyl) non-8-yn-1-yl)-3-methyl-1H-indazole-6-carboxamide (Intermediate 219, 0.23 g, 0.42 mmol) and tert-butyl 6-bromo-2-[(2R)-1-methylpyrrolidin-2-yl]pyrrolo[3,2-c]pyridine-1-carboxylate (Intermediate 1, 0.20 g, 0.52 mmol) in 1,4-dioxane (6 mL) was added cesium carbonate (0.43 g, 1.31 mmol). The reaction mixture was purged with argon gas for 15 min then Xantphos (0.094 g, 0.15 mmol) and Pd₂(dba)₃ (0.074 g, 0.078 mmol) were added. The reaction mixture was purged with argon gas for 10 min and heated at 100° C. for 2 h. The reaction mixture was then filtered through a celite bed and the filter cake was washed with ethyl acetate (50 mL). The filtrate was concentrated in vacuo and the crude residue was purified by combi-flash chromatography, by eluting with 90% ethyl acetate in heptane, to afford tert-butyl 6-(1-(9-(3-(((S)-2,6-dioxopiperidin-3-yl)carbamoyl)-2-fluorophenyl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 220, 0.15 g, 34%) as a yellow solid. Mass spec m/z 845.54 [M+H]⁺.

### Step 4

### Example 51: 1-(9-(3-(((S)-2,6-dioxopiperidin-3-yl)carbamoyl)-2-fluorophenyl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

To a cooled (0° C.) solution of tert-butyl 6-(1-(9-(3-(((S)-2,6-dioxopiperidin-3-yl)carbamoyl)-2-fluorophenyl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 220, 0.45 g, 0.53 mmol) in 1,4-dioxane (5 mL) was added 4M HCl in 1,4-dioxane (5 mL). The reaction mixture was stirred at room temperature for 3 h then was concentrated in vacuo. The crude material was purified by preparative HPLC (Method A) to afford 1-(9-(3-(((S)-2,6-dioxopiperidin-3-yl)carbamoyl)-2-fluorophenyl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 51, 0.040 g, 10%) as an off white solid. ¹H NMR (400 MHz, DMSO-d₆) δ ppm 1.30-1.39 (m, 6H), 1.49-1.55 (m, 2H), 1.83-1.92 (m, 5H), 1.96-2.11 (m, 3H), 2.18 (s, 3H), 2.25-2.31 (m, 2H), 2.43-2.46 (m, 3H), 2.54 (s, 3H), 2.73-2.78 (m, 1H), 3.13-3.18 (m, 1H), 4.41 (t, J=7.00 Hz, 2H), 4.73-4.77 (m, 1H), 6.41 (s, 1H), 7.24 (t, J=7.69 Hz, 1H), 7.53-7.60 (m, 2H), 7.71-7.75 (m, 1H), 7.76-7.80 (m, 1H), 8.25 (s, 1H), 8.44 (s, 1H), 8.53 (br s, 1H), 8.67-8.69 (m, 1H), 10.53 (br s, 1H), 10.86 (s, 1H), 11.39 (br s, 1H). Mass spec m/z 745.3 [M+H]⁺.

### Example 52 was Synthesized Following Scheme 51

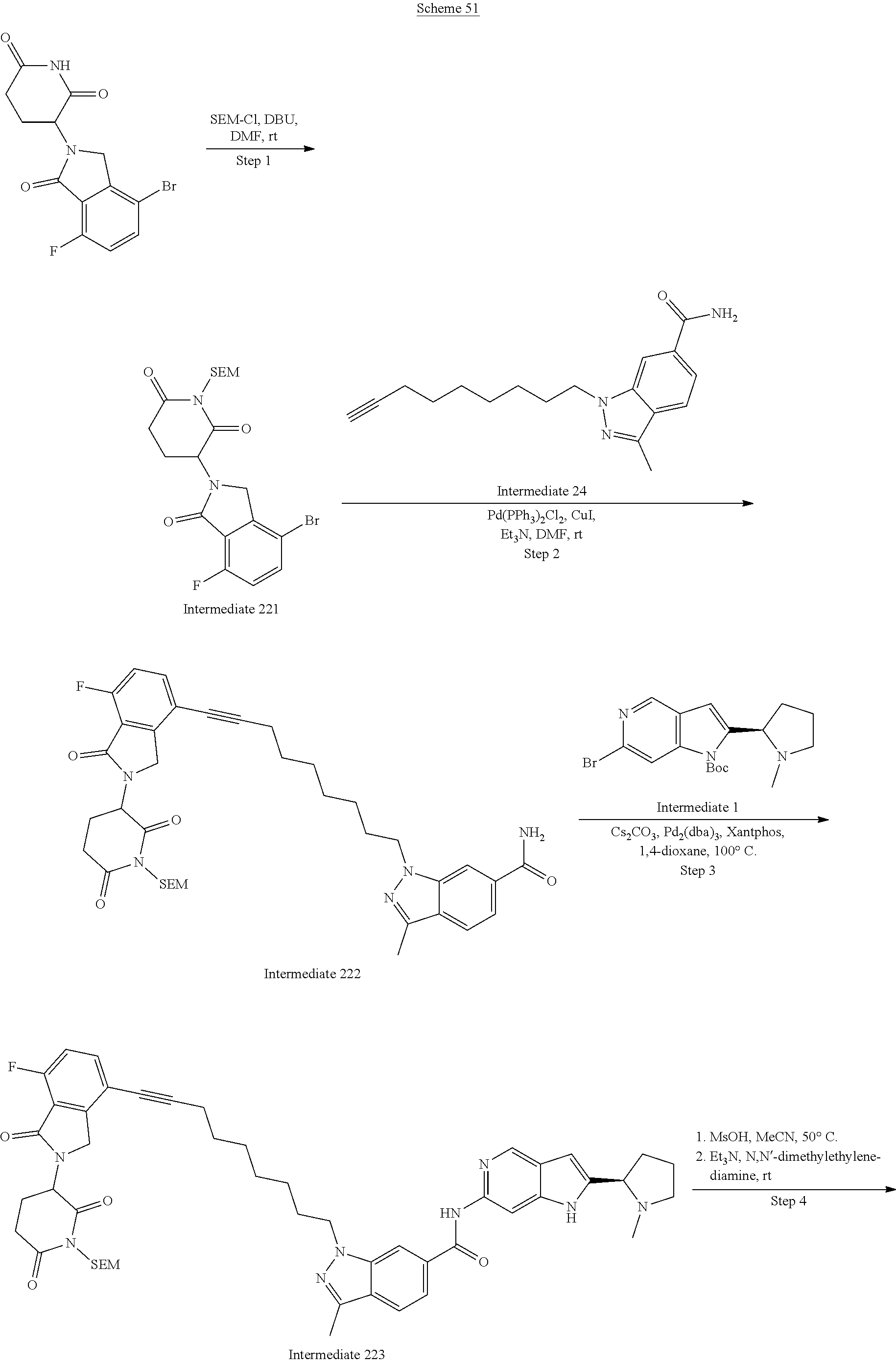 [SMILES: O=C1CCC(N2Cc3c(Br)ccc(F)c3C2=O)C(=O)N1 ; C[Si](C)(C)CCOCN1C(=O)CCC(N2Cc3c(Br)ccc(F)c3C2=O)C1=O ; C#CCCCCCCCn1nc(C)c2ccc(C(N)=O)cc21 ; Cc1nn(CCCCCCCC#Cc2ccc(F)c3c2CN(C2CCC(=O)N(COCC[Si](C)(C)C)C2=O)C3=O)c2cc(C(N)=O)ccc12 ; CN1CCC[C@@H]1c1cc2cnc(Br)cc2n1C(=O)OC(C)(C)C ; Cc1nn(CCCCCCCC#Cc2ccc(F)c3c2CN(C2CCC(=O)N(COCC[Si](C)(C)C)C2=O)C3=O)c2cc(C(=O)Nc3cc4[nH]c([C@H]5CCCN5C)cc4cn3)ccc12]

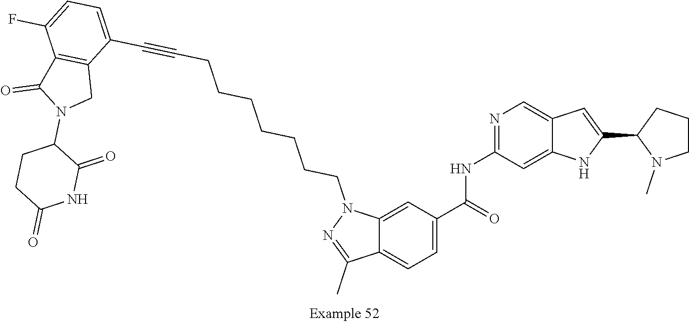 [SMILES: Cc1nn(CCCCCCCC#Cc2ccc(F)c3c2CN(C2CCC(=O)NC2=O)C3=O)c2cc(C(=O)Nc3cc4[nH]c([C@H]5CCCN5C)cc4cn3)ccc12]

### Step 1

### Intermediate 221: 3-(4-bromo-7-fluoro-1-oxoisoindolin-2-yl)-1-((2-(trimethylsilyl) ethoxy) methyl) piperidine-2,6-dione

To a cooled (0° C.) solution of 3-(4-bromo-7-fluoro-1-oxoisoindolin-2-yl)piperidine-2,6-dione (CAS No: 2438239-34-8, 1.00 g, 2.93 mmol) in dimethylformamide (10 mL) was added DBU (3.5 mL, 23 mmol) followed by 2-(trimethylsilyl)ethoxymethyl chloride (3.7 mL, 20 mmol) and the reaction mixture was stirred at room temperature for 16 h. The reaction mixture was quenched with ice-cold water (50 mL) and extracted with ethyl acetate (2×50 mL). The combined organic layers were dried over anhydrous Na₂SO₄, filtered and concentrated in vacuo. The crude material was purified by combi-flash column chromatography, by eluting with 20% ethyl acetate in heptane, to afford 3-(4-bromo-7-fluoro-1-oxoisoindolin-2-yl)-1-((2-(trimethylsilyl) ethoxy) methyl) piperidine-2,6-dione (Intermediate 221, 1.1 g, 80%) as a white solid. ¹H NMR (400 MHz, DMSO-d₆) δ ppm −0.02 (s, 9H), 0.78-0.88 (m, 2H), 2.05-2.08 (m, 1H), 2.39-2.43 (m, 1H), 2.76-2.82 (m, 1H), 3.02-3.10 (m, 1H), 3.47-3.58 (m, 2H), 4.20-4.25 (m, 1H), 4.42-4.46 (m, 1H), 5.02-5.09 (m, 2H), 5.22-5.26 (m, 1H), 7.35 (t, J=8.40 Hz, 1H), 7.89-7.92 (m, 1H). Mass spec m/z 471.0 [M+H]⁺.

### Step 2

### Intermediate 222: 1-(9-(2-(2,6-dioxo-1-((2-(trimethylsilyl) ethoxy) methyl) piperidin-3-yl)-7-fluoro-1-oxoisoindolin-4-yl) non-8-yn-1-yl)-3-methyl-1H-indazole-6-carboxamide

To a solution of 3-(4-bromo-7-fluoro-1-oxoisoindolin-2-yl)-1-((2-(trimethylsilyl)ethoxy)methyl) piperidine-2,6-dione (Intermediate 221, 1.6 g, 3.4 mmol) and 3-methyl-1-(non-8-yn-1-yl)-1H-indazole-6-carboxamide (Intermediate 24, 1.00 g, 3.36 mmol) in dimethylformamide (15 mL) was added triethylamine (1.03 g, 10.1 mmol). The reaction mixture was purged with argon for 15 min then PdCl₂(PPh₃)₂ (0.48 g, 0.67 mmol) and copper (I) iodide (0.064 g, 0.34 mmol) were added. The reaction mixture was purged with argon for another 5 min and stirred at room temperature for 2 h. The reaction mixture was diluted with water (200 mL) and extracted with ethyl acetate (3×100 mL). The combined organic layers were separated, dried over anhydrous Na₂SO₄, filtered and concentrated in vacuo. The crude material was purified by combi-flash chromatography, by eluting with 80% ethyl acetate in heptane, to afford 1-(9-(2-(2,6-dioxo-1-((2-(trimethylsilyl) ethoxy) methyl) piperidin-3-yl)-7-fluoro-1-oxoisoindolin-4-yl) non-8-yn-1-yl)-3-methyl-1H-indazole-6-carboxamide (Intermediate 222, 0.90 g, 39%) as a yellow solid. ¹H NMR (400 MHz, DMSO-d₆) δ ppm −0.04 (s, 9H), 0.78-0.87 (m, 2H), 1.25-1.40 (m, 6H), 1.48-1.56 (m, 2H), 1.78-1.86 (m, 2H), 2.02-2.08 (m, 1H), 2.40-2.45 (m, 2H), 2.48 (s, 3H), 2.73-2.80 (m, 2H), 3.01-3.10 (m, 1H), 3.49-3.56 (m, 2H), 4.23-4.36 (m, 3H), 4.43-4.48 (m, 1H), 5.00-5.08 (m, 2H), 5.21-5.25 (m, 1H), 7.30 (t, J=8.80 Hz, 1H), 7.41 (br s, 1H), 7.58 (d, J=8.40 Hz, 1H), 7.63-7.67 (m, 1H), 7.72 (d, J=8.40 Hz, 1H), 8.02 (br s, 1H), 8.11 (s, 1H). Mass spec m/z 687.9 [M+H]⁺.

### Step 3

### Intermediate 223: tert-butyl 6-(1-(9-(2-(2,6-dioxo-1-((2-(trimethylsilyl)ethoxy)methyl)piperidin-3-yl)-7-fluoro-1-oxoisoindolin-4-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(9-(2-(2,6-dioxo-1-((2-(trimethylsilyl)ethoxy)methyl)piperidin-3-yl)-7-fluoro-1-oxoisoindolin-4-yl) non-8-yn-1-yl)-3-methyl-1H-indazole-6-carboxamide (Intermediate 222, 0.57 g, 0.84 mmol) in 1,4-dioxane (2 mL) was added tert-butyl (R)-6-bromo-2-(1-methylpyrrolidin-2-yl)-1H-pyrrolo[3,2-c]pyridine-1-carboxylate (Intermediate 1, 0.40 g, 1.05 mmol) followed by cesium carbonate (0.61 g, 3.15 mmol). The reaction mixture was purged with argon for 15 min. To the reaction mixture was added Pd₂(dba)₃ (0.15 g, 0.16 mmol) followed by Xantphos (0.18 g, 0.31 mmol) and the mixture was purged with argon gas for another 10 min then heated at 100° C. for 2 h under argon atmosphere. The reaction mixture was filtered through a celite bed and the filter pad was washed with ethyl acetate (50 mL). The filtrate was concentrated in vacuo and the crude material purified by combi-flash column chromatography, by eluting with 90% ethyl acetate in heptane, to afford tert-butyl 6-(1-(9-(2-(2,6-dioxo-1-((2-(trimethylsilyl)ethoxy)methyl)piperidin-3-yl)-7-fluoro-1-oxoisoindolin-4-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 223, 0.35 g, 34%) as a yellow solid. ¹H NMR (400 MHz, DMSO-d₆) δ ppm −0.05 (s, 9H), 0.79-0.90 (m, 2H), 1.07-1.15 (m, 2H), 1.23-1.44 (m, 6H), 1.49-1.54 (m, 2H), 1.61-1.65 (m, 3H), 1.70 (s, 9H), 1.72-1.76 (m, 3H), 1.82-1.86 (m, 2H), 1.98-2.08 (m, 1H), 2.33 (s, 3H), 2.36-2.41 (m, 1H), 2.43 (s, 3H), 3.04-3.18 (m, 2H), 3.35-3.38 (m, 1H), 3.48-3.57 (m, 1H), 3.90-3.93 (m, 1H), 4.22-4.46 (m, 4H), 4.92-5.12 (m, 3H), 6.76 (s, 1H), 6.97-7.05 (m, 1H), 7.26-7.33 (m, 1H), 7.58-7.61 (m, 1H), 7.71-7.79 (m, 1H), 8.42 (s, 1H), 8.60 (s, 1H), 8.97 (s, 1H), 10.73 (br s, 1H). Mass spec m/z 987.0 [M+H]⁺.

### Step 4

### Example 52: 1-(9-(2-(2,6-dioxopiperidin-3-yl)-7-fluoro-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(9-(2-(2,6-dioxo-1-((2-(trimethylsilyl)ethoxy)methyl)piperidin-3-yl)-7-fluoro-1-oxoisoindolin-4-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 223, 0.30 g, 0.30 mmol) in acetonitrile (10 mL) was added methanesulfonic acid (0.20 mL, 3.04 mmol) and the reaction mixture was heated at 50° C. for 2 h. After cooling to room temperature, N,N′-dimethylethylenediamine (0.18 mL, 1.52 mmol) was added followed by triethylamine (0.85 mL, 6.07 mmol) and the reaction mixture was stirred at room temperature for 3 h. The reaction mixture was quenched with water (20 mL) and the resultant precipitate was collected by filtration and dried in vacuo. The crude material was purified preparative HPLC (Method A) to afford 1-(9-(2-(2,6-dioxopiperidin-3-yl)-7-fluoro-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 52, 0.045 g, 19%) as an off-white solid. ¹H NMR (400 MHz, DMSO-d₆) δ ppm 1.29-1.42 (m, 6H), 1.50-1.57 (m, 2H), 1.84-2.00 (m, 6H), 2.12-2.16 (m, 1H), 2.17 (s, 3H), 2.24-2.30 (m, 1H), 2.39-2.41 (m, 1H), 2.43 (s, 3H), 2.45-2.47 (m, 1H), 2.54-2.60 (m, 1H), 2.84-2.94 (m, 1H), 3.13-3.18 (m, 1H), 3.32-3.36 (m, 2H), 4.27-4.32 (m, 1H), 4.38-4.45 (m, 3H), 5.07-5.11 (m, 1H), 6.40 (s, 1H), 7.27 (t, J=8.94 Hz, 1H), 7.60-7.64 (m, 1H), 7.71-7.75 (m, 1H), 7.76-7.80 (m, 1H), 8.25 (s, 1H), 8.43 (s, 1H), 8.52 (s, 1H), 10.51 (s, 1H), 11.01 (s, 1H), 11.38 (s, 1H). Mass spec m/z 757.3 [M+H]⁺.

### Example 53: N-(3-chloro-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide

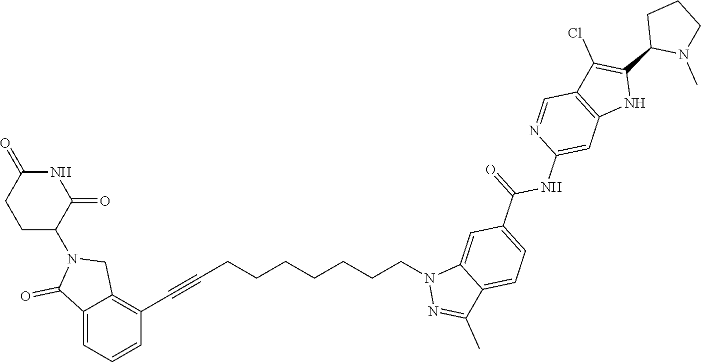 [SMILES: Cc1nn(CCCCCCCC#Cc2cccc3c2CN(C2CCC(=O)NC2=O)C3=O)c2cc(C(=O)Nc3cc4[nH]c([C@H]5CCCN5C)c(Cl)c4cn3)ccc12]

To a solution of 1-(9-(2-(2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 1, 0.10 g, 0.13 mmol) in dimethylformamide (3 mL) was added N-chlorosuccinimide (0.027 g, 0.20 mmol) and the reaction mixture was stirred for 30 h. The reaction mixture was poured into ice cold water (50 mL), the resultant precipitate was collected by filtration and dried in vacuo. The crude material was purified by preparative HPLC (Method A) to afford N-(3-chloro-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide (Example 53, 0.016 g, 15%) as an off white solid. ¹H NMR (400 MHz, DMSO-d₆) δ ppm 1.32-1.38 (m, 6H), 1.52-1.58 (m, 2H), 1.82-1.91 (m, 4H), 1.98-2.07 (m, 2H), 2.17 (s, 3H), 2.30-2.36 (m, 2H), 2.42-2.45 (m, 2H), 2.47 (s, 3H), 2.56-2.59 (m, 1H), 2.85-2.94 (m, 1H), 3.16-3.20 (m, 2H), 3.51-3.55 (m, 1H), 4.27-4.31 (m, 1H), 4.39-4.45 (m, 3H), 5.10-5.15 (m, 1H), 7.47-7.51 (m, 1H), 7.58-7.60 (m, 1H), 7.68-7.74 (m, 2H), 7.76-7.80 (m, 1H), 8.31 (s, 1H), 8.44 (s, 1H), 8.52 (s, 1H), 10.67 (s, 1H), 10.99 (s, 1H), 11.70 (br s, 1H). Mass spec m/z 773.2 [M+H]⁺.

### Example 54 was Synthesized Following Scheme 52

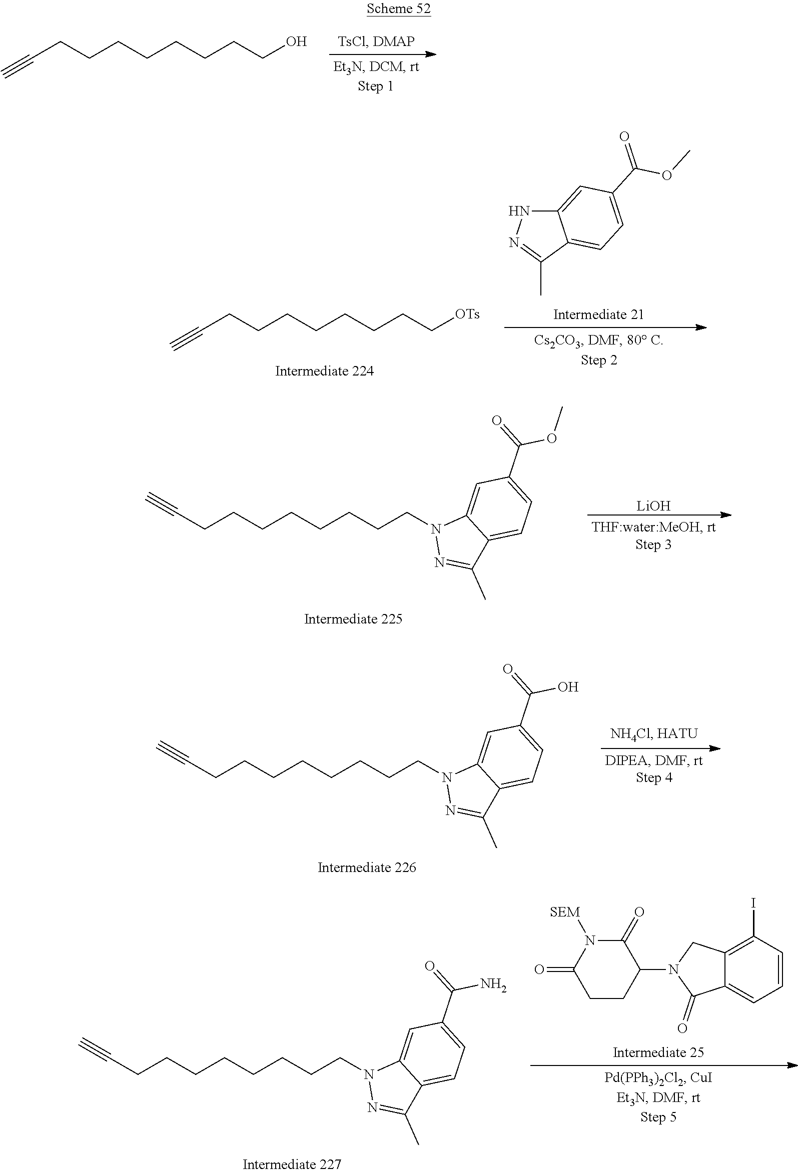

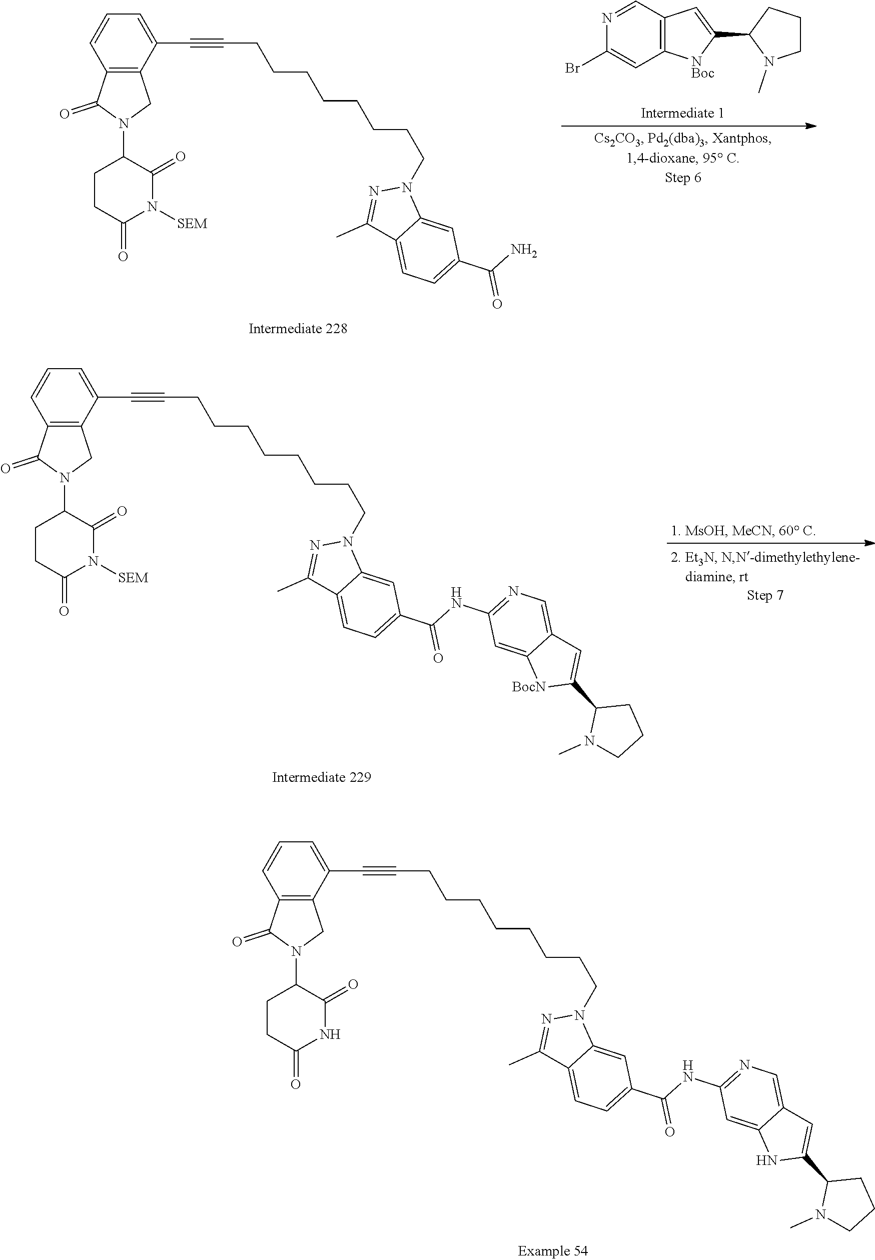 [SMILES: Cc1nn(CCCCCCCCC#Cc2cccc3c2CN(C2CCC(=O)N(COCC[Si](C)(C)C)C2=O)C3=O)c2cc(C(N)=O)ccc12 ; CN1CCC[C@@H]1c1cc2cnc(Br)cc2n1C(=O)OC(C)(C)C ; Cc1nn(CCCCCCCCC#Cc2cccc3c2CN(C2CCC(=O)N(COCC[Si](C)(C)C)C2=O)C3=O)c2cc(C(=O)Nc3cc4c(cn3)cc([C@H]3CCCN3C)n4C(=O)OC(C)(C)C)ccc12 ; Cc1nn(CCCCCCCCC#Cc2cccc3c2CN(C2CCC(=O)NC2=O)C3=O)c2cc(C(=O)Nc3cc4[nH]c([C@H]5CCCN5C)cc4cn3)ccc12]

### Step 1

### Intermediate 224: dec-9-yn-1-yl 4-methylbenzenesulfonate

To a cooled (0° C.) solution of dec-9-yn-1-ol (CAS No: 13019-22-2, 4.0 g, 26 mmol) in dichloromethane (50 mL) was added p-toluenesulfonyl chloride (5.8 mL, 39 mmol) and triethylamine (8.3 g, 78 mmol) followed by 4-dimethylaminopyridine (0.33 g, 2.6 mmol). The reaction mixture was stirred at room temperature for 16 h then poured into ice cold water (150 mL) and extracted with dichloromethane (2×100 mL). The combined organic layers were separated, dried over anhydrous Na₂SO₄, and concentrated in vacuo. The crude material was purified by combi-flash chromatography, by eluting with 20% ethyl acetate in heptane, to afford dec-9-yn-1-yl-4-methylbenzenesulfonate (Intermediate 224, 4.5 g, 56%) as a yellow liquid. ¹H NMR (400 MHz, DMSO-d₆) δ ppm 1.11-1.30 (m, 8H), 1.36-1.44 (m, 2H), 1.49-1.58 (m, 2H), 2.09-2.16 (m, 2H), 2.42 (s, 3H), 2.72 (s, 1H), 4.00 (t, J=6.22 Hz, 2H), 7.48 (d, J=7.88 Hz, 2H), 7.78 (d, J=8.29 Hz, 2H).

### Step 2

### Intermediate 225: methyl 1-(dec-9-yn-1-yl)-3-methyl-1H-indazole-6-carboxylate

To a solution of dec-9-yn-1-yl-4-methylbenzenesulfonate (Intermediate 224, 2.9 g, 9.5 mmol) in dimethylformamide (15 mL) was added cesium carbonate (8.1 g, 24 mmol) and methyl 3-methyl-1H-indazole-6-carboxylate (Intermediate 21, 1.5 g, 7.9 mmol). The reaction mixture was heated at 80° C. for 16 h then poured into ice cold water (120 mL) and extracted with ethyl acetate (2×120 mL). The combined organic layers were separated, dried over Na₂SO₄, and concentrated in vacuo to obtain a crude mixture of regioisomers. The regioisomers were separated by combi-flash chromatography, by eluting with 30% ethyl acetate in heptane, to afford desired regioisomer methyl-1-(dec-9-yn-1-yl)-3-methyl-1H-indazole-6-carboxylate (Intermediate 225, 1.60 g, 62%) as an off white solid. ¹H NMR (400 MHz, DMSO-d₆) δ ppm 1.16-1.32 (m, 10H), 1.36-1.43 (m, 2H), 1.75-1.84 (m, 2H), 2.08-2.13 (m, 2H), 2.39-2.42 (m, 1H), 2.70 (s, 1H), 3.90 (s, 3H), 4.40 (t, J=6.91 Hz, 2H), 7.65 (d, J=8.33 Hz, 1H), 7.81 (d, J=8.33 Hz, 1H), 8.22 (s, 1H). Mass spec m/z 327.28 [M+H]⁺.

### Step 3

### Intermediate 226: 1-(dec-9-yn-1-yl)-3-methyl-1H-indazole-6-carboxylic acid

To a solution of methyl-1-(dec-9-yn-1-yl)-3-methyl-1H-indazole-6-carboxylate (Intermediate 225, 1.9 g, 5.8 mmol) in tetrahydrofuran (6 mL), methanol (6 mL) and water (6 mL) was added lithium hydroxide (0.43 g, 17 mmol) and the reaction mixture was stirred for 16 h. The reaction mixture was concentrated in vacuo and the crude residue was adjusted to pH~3 by the addition of 1N aqueous HCl. The resultant precipitate was collected by filtration, washed with heptane (30 mL) and dried in vacuo to afford 1-(dec-9-yn-1-yl)-3-methyl-1H-indazole-6-carboxylic acid (Intermediate 226, 1.75 g, 96%) as an off white solid. ¹H NMR (400 MHz, DMSO-d₆) δ ppm 1.16-1.31 (m, 8H), 1.34-1.41 (m, 2H), 1.75-1.85 (m, 2H), 2.08-2.12 (m, 2H), 2.52 (s, 3H), 2.62 (t, J=2.63 Hz, 1H), 4.37 (t, J=6.82 Hz, 2H), 7.67 (d, J=8.44, 1H), 7.79 (d, J=8.38 Hz, 1H), 8.14 (s, 1H). Mass spec m/z 313.23 [M+H]⁺.

### Step 4

### Intermediate 227: 1-(dec-9-yn-1-yl)-3-methyl-1H-indazole-6-carboxamide

To a cooled (0° C.) solution of 1-(dec-9-yn-1-yl)-3-methyl-1H-indazole-6-carboxylic acid (Intermediate 226, 1.80 g, 5.76 mmol) in dimethylformamide (20 mL) was added HATU (5.65 g, 14.4 mmol) and DIPEA (5.03 mL, 28.8 mmol) and the mixture was stirred for 10 min. Ammonium chloride (1.01 mL, 28.8 mmol) was then added and the reaction mixture was stirred at room temperature for 6 h. The reaction mixture was poured into ice cold water (200 mL), the resultant precipitate was collected by filtration, washed with heptane (50 mL) and dried in vacuo to afford 1-(dec-9-yn-1-yl)-3-methyl-1H-indazole-6-carboxamide (Intermediate 227, 1.70 g, 95%) as an off white solid which was used in the next step without further purification. ¹H NMR (400 MHz, DMSO-d₆) δ ppm 1.09-1.27 (m, 7H), 1.29-1.38 (m, 2H), 1.72-1.80 (m, 2H), 2.02-2.08 (m, 2H), 2.46 (s, 3H), 2.56-2.58 (m, 1H), 4.30 (t, J=6.58 Hz, 2H), 7.55 (d, J=8.33 Hz, 1H), 7.73 (d, J=8.33 Hz, 1H), 8.05 (s, 1H). Mass spec m/z 312.27 [M+H]⁺.

### Step 5

### Intermediate 228: 1-(10-(2-(2,6-dioxo-1-((2-(trimethylsilyl) ethoxy) methyl) piperidin-3-yl)-1-oxoisoindolin-4-yl) dec-9-yn-1-yl)-3-methyl-1H-indazole-6-carboxamide

To a solution of 1-(dec-9-yn-1-yl)-3-methyl-1H-indazole-6-carboxamide (Intermediate 227, 1.0, 3.2 mmol) in dimethylformamide (1 mL) was added 3-(4-iodo-1-oxoisoindolin-2-yl)-1-((2-(trimethylsilyl)ethoxy)methyl)piperidine-2,6-dione (Intermediate 25, 1.6 g, 3.2 mmol) followed by triethylamine (20 mL, 120 mmol). The reaction mixture was purged with argon for 15 min followed by the addition of copper (I) iodide (0.06 g, 0.32 mmol) and Pd(PPh₃)₂Cl₂ (0.24 g, 0.32 mmol). The reaction mixture was purged with argon for 10 min and stirred at room temperature for 1 h. The reaction mixture was poured into ice cold water (100 mL) and extracted with ethyl acetate (2×150 mL). The combined organic layers were separated, dried over anhydrous Na₂SO₄ and concentrated in vacuo. The crude material was purified by combi-flash column chromatography, by eluting with 7% MeOH in DCM, to afford 1-(10-(2-(2,6-dioxo-1-((2-(trimethylsilyl)ethoxy)methyl)piperidin-3-yl)-1-oxoisoindolin-4-yl)dec-9-yn-1-yl)-3-methyl-1H-indazole-6-carboxamide (Intermediate 228, 1.3 g, 59%) as a grey solid. ¹H NMR (400 MHz, DMSO-d₆) δ ppm −0.04 (s, 9H), 0.78-0.86 (m, 2H), 1.16-1.46 (m, 9H), 1.48-1.56 (m, 2H), 1.75-1.83 (m, 2H), 2.03-2.10 (m, 1H), 2.36 (m, 1H), 2.39 (s, 3H), 2.73-2.80 (m, 2H), 3.02-3.10 (m, 1H), 3.48-3.57 (m, 2H), 4.21-4.34 (m, 3H), 4.43-4.47 (m, 1H), 4.99-5.10 (m, 2H), 5.25-5.29 (m, 1H), 7.41 (br s, 1H), 7.50-7.54 (m, 1H), 7.58-7.63 (m, 2H), 7.71-7.72 (m, 2H), 8.02 (br s, 1H), 8.11 (s, 1H). Mass spec m/z 684.51 [M+H]⁺.

### Step 6

### Intermediate 229: tert-butyl-6-(1-(10-(2-(2,6-dioxo-1-((2-(trimethylsilyl)ethoxy)methyl)piperidin-3-yl)-1-oxoisoindolin-4-yl)dec-9-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(10-(2-(2,6-dioxo-1-((2-(trimethylsilyl)ethoxy)methyl)piperidin-3-yl)-1-oxoisoindolin-4-yl)dec-9-yn-1-yl)-3-methyl-1H-indazole-6-carboxamide (Intermediate 228, 0.52 g, 0.75 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 1, 0.36 g, 0.94 mmol) followed by cesium carbonate (0.48 g, 1.42 mmol). The reaction mixture was purged with argon for 15 min then Xantphos (0.086 g, 0.14 mmol) and Pd₂(dba)₃ (0.12 g, 0.14 mmol) were added. The reaction mixture was purged with argon gas for 10 min and then heated at 95° C. for 1 h. The reaction mixture was filtered through a celite bed and the filter pad was washed with DCM (30 mL). The filtrate was concentrated in vacuo to afford tert-butyl 6-(1-(10-(2-(2,6-dioxo-1-((2-(trimethylsilyl)ethoxy)methyl)piperidin-3-yl)-1-oxoisoindolin-4-yl)dec-9-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 229, 0.7 g, 75%) as a liquid which was used for the next step without further purification. Mass spec m/z 983.65 [M+H]⁺.

### Step 7

### Example 54: 1-(10-(2-(2,6-dioxopiperidin-3-yl)-1-oxoisoindolin-4-yl) dec-9-yn-1-yl)-3-methyl-N-(2-((R)-1-methylpyrrolidin-2-yl)-1H-pyrrolo[3,2-c]pyridin-6-yl)-1H-indazole-6-carboxamide

To a cooled (0° C.) solution of tert-butyl-6-(1-(10-(2-(2,6-dioxo-1-((2-(trimethylsilyl)ethoxy)methyl)piperidin-3-yl)-1-oxoisoindolin-4-yl)dec-9-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 229, 0.50 g, 0.51 mmol) in acetonitrile (8 mL) was added methanesulfonic acid (0.51 g, 5.08 mmol) and the reaction mixture was then heated at 60° C. for 2 h. After cooling to room temperature, N,N′-dimethylethylenediamine (0.28 g, 3.05 mmol) followed by triethylamine (1.08 g, 10.17 mmol) were added the reaction mixture was stirred at room temperature for 16 h. The reaction mixture was poured into ice cold water (120 mL), the resultant precipitate was collected by filtration and dried in vacuo. The crude material was purified by preparative HPLC (Method A) to afford 1-(10-(2-(2,6-dioxopiperidin-3-yl)-1-oxoisoindolin-4-yl)dec-9-yn-1-yl)-3-methyl-N-(2-((R)-1-methylpyrrolidin-2-yl)-1H-pyrrolo[3,2-c]pyridin-6-yl)-1H-indazole-6-carboxamide (Example 54, 0.02 g, 5%) as an off white solid. ¹H NMR (400 MHz, DMSO-d₆) δ ppm 1.24-1.43 (m, 9H), 1.51-1.58 (m, 2H), 1.77-1.93 (m, 5H), 1.96-2.02 (m, 1H), 2.13-2.15 (m, 1H), 2.17 (s, 3H), 2.24-2.31 (m, 1H), 2.41-2.46 (m, 4H), 2.52-2.62 (m, 2H), 2.85-2.95 (m, 1H), 3.13-3.16 (m, 1H), 3.32-3.36 (m, 1H), 4.26-4.32 (m, 1H), 4.38-4.44 (m, 3H), 5.11-5.15 (m, 1H), 6.40 (s, 1H), 7.48-7.53 (m, 1H), 7.58-7.62 (m, 1H), 7.68-7.79 (m, 3H), 8.25 (s, 1H), 8.42 (s, 1H), 8.52 (s, 1H), 10.52 (s, 1H), 11.00 (s, 1H), 11.38 (s, 1H). Mass spec m/z 753.3 [M+H]⁺.

### Example 55 was Synthesized Following Scheme 53

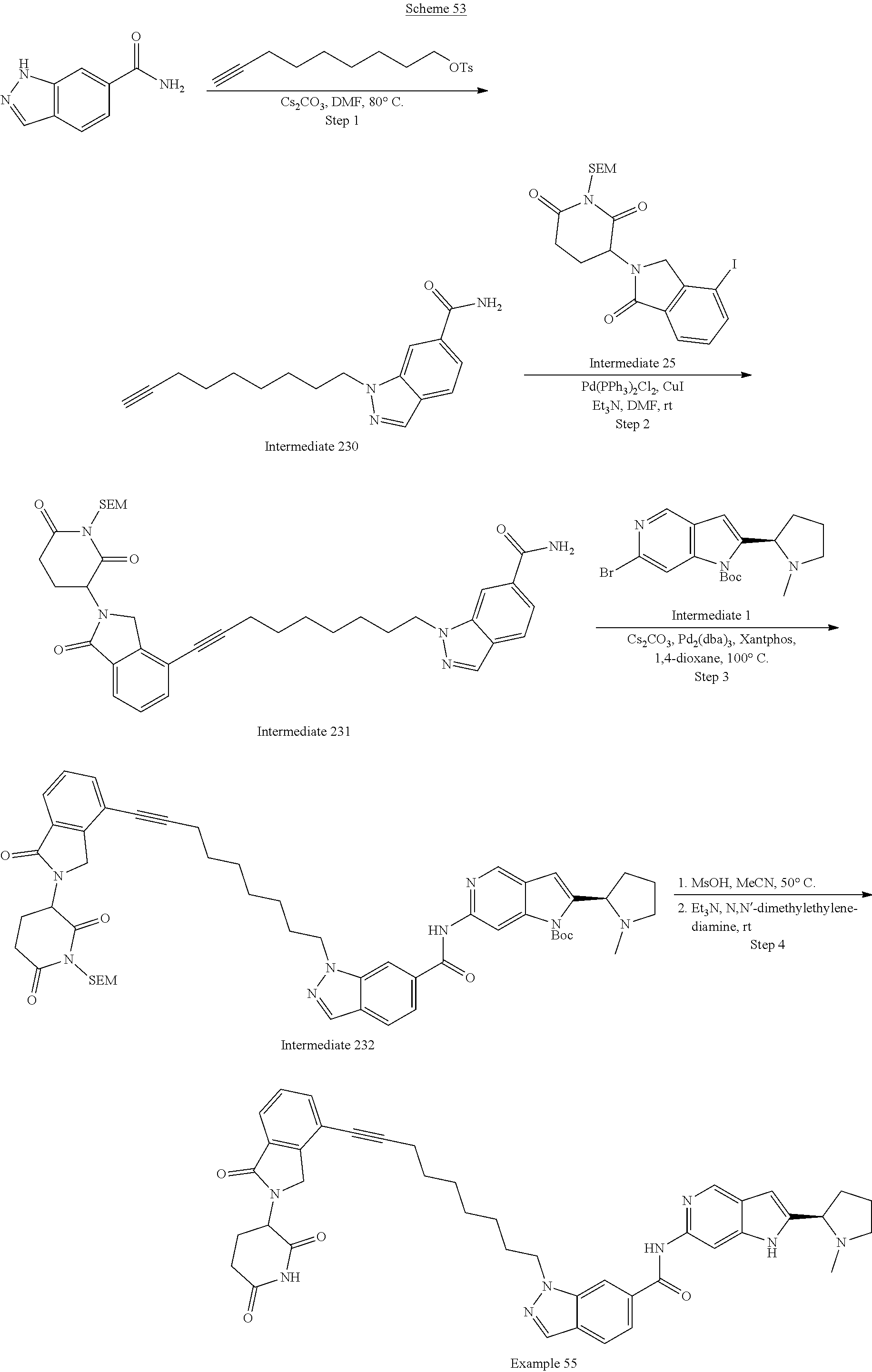

### Step 1

### Intermediate 230: 1-(non-8-yn-1-yl)-1H-indazole-6-carboxamide

To solution of 1H-indazole-6-carboxamide (CAS No: 906000-44-0, 3.00 g, 18.61 mmol) in dimethylformamide (40 mL) was added cesium carbonate (18.3 g, 56.1 mmol) followed by non-8-ynyl-4-methylbenzenesulfonate (CAS No: 87462-64-4, 6.58 g, 22.4 mmol) and the reaction mixture was heated at 80° C. for 2 h. The reaction mixture was diluted with cold water (100 mL) and extracted with ethyl acetate (3×100 mL). The combined organic layers were washed with brine solution (100 mL), separated, dried over anhydrous Na₂SO₄, filtered and concentrated in vacuo to obtain crude compound as a mixture of regioisomers. The regioisomers were separated by combi-flash chromatography, by eluting with 55% ethyl acetate in heptane, to afford desired regioisomer 1-(non-8-yn-1-yl)-1H-indazole-6-carboxamide (Intermediate 230, 2.10 g, 40%) as an off white solid. ¹H NMR (400 MHz, DMSO-d₆) δ ppm 1.20-1.42 (m, 8H), 1.80-1.90 (m, 2H), 2.08-2.12 (m, 2H), 2.70 (s, 1H), 4.43 (t, J=6.43 Hz, 2H), 7.44 (br s, 1H), 7.63 (d, J=8.29 Hz, 1H), 7.79 (d, J=8.29 Hz, 1H), 8.05 (br s, 1H), 8.11 (s, 1H), 8.21 (s, 1H). Mass spec m/z 284.0 [M+H]⁺.

### Step 2

### Intermediate 231: 1-(9-(2-(2,6-dioxo-1-((2-(trimethylsilyl) ethoxy) methyl) piperidin-3-yl)-1-oxoisoindolin-4-yl) non-8-yn-1-yl)-1H-indazole-6-carboxamide

To a solution of 1-(non-8-yn-1-yl)-1H-indazole-6-carboxamide (Intermediate 230, 1.00 g, 3.53 mmol) in dimethylformamide (8 mL) was added 3-(4-iodo-1-oxoisoindolin-2-yl)-1-((2-(trimethylsilyl)ethoxy)methyl)piperidine-2,6-dione (Intermediate 25, 1.95 g, 3.90 mmol) followed by triethylamine (12.7 g, 125 mmol) and the reaction mixture was purged with argon gas for 10 min. To the reaction mixture was added PdCl₂(PPh₃)₂ (0.25 g, 0.36 mmol) followed by copper (I) iodide (0.070 g, 0.36 mmol). The reaction mixture was purged with argon for a further 5 min and then stirred at room temperature for 1 h. The reaction mixture was diluted with water (60 mL) and extracted with ethyl acetate (3×50 mL). The combined organic layers were washed with brine solution (2×50 mL), separated, dried over anhydrous Na₂SO₄, filtered, and concentrated in vacuo. The crude material was purified by combi-flash column chromatography, by eluting with 2% MeOH in DCM, to afford 1-(9-(2-(2,6-dioxo-1-((2-(trimethylsilyl) ethoxy) methyl) piperidin-3-yl)-1-oxoisoindolin-4-yl) non-8-yn-1-yl)-1H-indazole-6-carboxamide (Intermediate 231, 1.00 g, 43%) as a white solid. ¹H NMR (400 MHz, DMSO-d₆) δ ppm −0.04 (s, 9H), 0.76-0.88 (m, 2H), 1.22-1.44 (m, 6H), 1.51-1.54 (m, 2H), 1.80-1.88 (m, 2H), 2.01-2.07 (m, 1H), 2.38-2.45 (m, 3H), 2.76-2.81 (m, 1H), 3.00-3.12 (m, 1H), 3.49-3.54 (m, 2H), 4.23-4.27 (m, 1H), 4.41-4.47 (m, 3H), 5.00-5.09 (m, 2H), 5.24-5.29 (m, 1H), 7.42 (br s, 1H), 7.49-7.53 (m, 1H), 7.61-7.65 (m, 2H), 7.71 (d, J=7.46 Hz, 1H), 7.78 (d, J=8.29 Hz, 1H), 8.03 (br s, 1H), 8.09 (s, 1H), 8.20 (s, 1H). Mass spec m/z 656.0 [M+H]⁺.

### Step 3

### Intermediate 232: tert-butyl-6-(1-(9-(2-(2,6-dioxo-1-((2-(trimethylsilyl)ethoxy)methyl)piperidin-3-yl)-1-oxoisoindolin-4-yl)non-8-yn-1-yl)-1H-indazole-6-carboxamido)-2-((R)-1-methylpyrrolidin-2-yl)-1H-pyrrolo[3,2-c]pyridine-1-carboxylate

To a solution of 1-(9-(2-(2,6-dioxo-1-((2-(trimethylsilyl)ethoxy)methyl)piperidin-3-yl)-1-oxoisoindolin-4-yl) non-8-yn-1-yl)-1H-indazole-6-carboxamide (Intermediate 231, 0.70 g, 1.06 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 1, 0.49 g, 1.28 mmol) followed by cesium carbonate (1.08 g, 3.31 mmol). The reaction mixture was purged with argon for 5 min. To the reaction mixture was added Pd₂(dba)₃ (0.15 g, 0.16 mmol) followed by Xantphos (0.19 g, 0.32 mmol) and the mixture was purged with argon for a further 5 min then heated at 100° C. for 2 h. The reaction mixture was diluted with water (60 mL) and extracted with ethyl acetate (3×80 mL). The combined organic layers were washed with brine solution (2×50 mL), separated, dried over anhydrous Na₂SO₄, filtered, and concentrated in vacuo. The crude material was purified by combi-flash column chromatography, by eluting with 2% MeOH in DCM, to afford tert-butyl-6-(1-(9-(2-(2,6-dioxo-1-((2-(trimethylsilyl)ethoxy)methyl)piperidin-3-yl)-1-oxoisoindolin-4-yl)non-8-yn-1-yl)-1H-indazole-6-carboxamido)-2-((R)-1-methylpyrrolidin-2-yl)-1H-pyrrolo[3,2-c]pyridine-1-carboxylate (Intermediate 232, 0.46 g, 45%) as a yellow solid. ¹H NMR (400 MHz, DMSO-d₆) δ ppm −0.06 (s, 9H), 0.76-0.86 (m, 2H), 1.22-1.44 (m, 7H), 1.48-1.57 (m, 2H), 1.60-1.64 (m, 1H), 1.70 (s, 9H), 1.73-1.80 (m, 2H), 1.86-1.93 (m, 2H), 2.00-2.08 (m, 1H), 2.31-2.33 (m, 2H), 2.36 (s, 3H), 2.41-2.47 (m, 3H), 2.73-2.80 (m, 1H), 3.01-3.16 (m, 2H), 3.46-3.53 (m, 2H), 4.21-4.26 (m, 1H), 4.41-4.51 (m, 3H), 4.99-5.07 (m, 2H), 5.25-5.29 (m, 1H), 6.76 (s, 1H), 7.47-7.51 (m, 1H), 7.60 (d, J=7.45 Hz, 1H), 7.70 (d, J=7.45 Hz, 1H), 7.75 (d, J=8.77 Hz, 1H), 7.81-7.87 (m, 1H), 8.14 (s, 1H), 8.52 (s, 1H), 8.61 (s, 1H), 8.98 (s, 1H), 10.80 (s, 1H). Mass spec m/z 955.1 [M+H]⁺.

### Step 4

### Example 55: 1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrol[3,2-c]pyridin-6-yl)-1H-indazole-6-carboxamide

To a solution of tert-butyl-6-(1-(9-(2-(2,6-dioxo-1-((2-(trimethylsilyl)ethoxy)methyl)piperidin-3-yl)-1-oxoisoindolin-4-yl)non-8-yn-1-yl)-1H-indazole-6-carboxamido)-2-((R)-1-methylpyrrolidin-2-yl)-1H-pyrrolo[3,2-c]pyridine-1-carboxylate (Intermediate 232, 0.45 g, 0.47 mmol) in acetonitrile (8 mL) was added methanesulfonic acid (0.31 mL, 4.71 mmol) at room temperature and the reaction mixture was heated at 50° C. for 2 h. The reaction mixture was cooled to room temperature, then N,N′-dimethylethylenediamine (0.34 mL, 2.82 mmol) followed by triethylamine (1.32 mL, 9.42 mmol) were added and the mixture stirred at room temperature for 2 h. The reaction mixture was concentrated in vacuo and the obtained solid was washed with ether (2×50 mL) and DCM/Pentanes (2×40 mL) and then dried in vacuo. The crude material was purified by preparative HPLC (Method A) to afford 1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrolo[3,2-c]pyridin-6-yl)-1H-indazole-6-carboxamide (Example 55, 0.08 g, 23%) as an off white solid. ¹H NMR (400 MHz, DMSO-d₆) δ ppm 1.28-1.42 (m, 6H), 1.51-1.58 (m, 2H), 1.79-2.02 (m, 6H), 2.17 (s, 3H), 2.24-2.30 (m, 1H), 2.42-2.46 (m, 3H), 2.58-2.62 (m, 1H), 2.85-2.94 (m, 2H), 3.13-3.18 (m, 2H), 4.26-4.32 (m, 1H), 4.39-4.46 (m, 1H), 4.50 (t, J=6.91 Hz, 2H), 5.11-5.15 (m, 1H), 6.40 (s, 1H), 7.46-7.52 (m, 1H), 7.60 (d, J=7.58, 1H), 7.67-7.71 (m, 2H), 7.75-7.78 (m, 1H), 7.82-7.86 (m, 1H), 8.13 (s, 1H), 8.25 (s, 1H), 8.52 (s, 1H), 10.56 (s, 1H), 11.39 (s, 1H). Mass spec m/z 725.3 [M+H]⁺.

### Example 56 was Synthesized Following Scheme 54

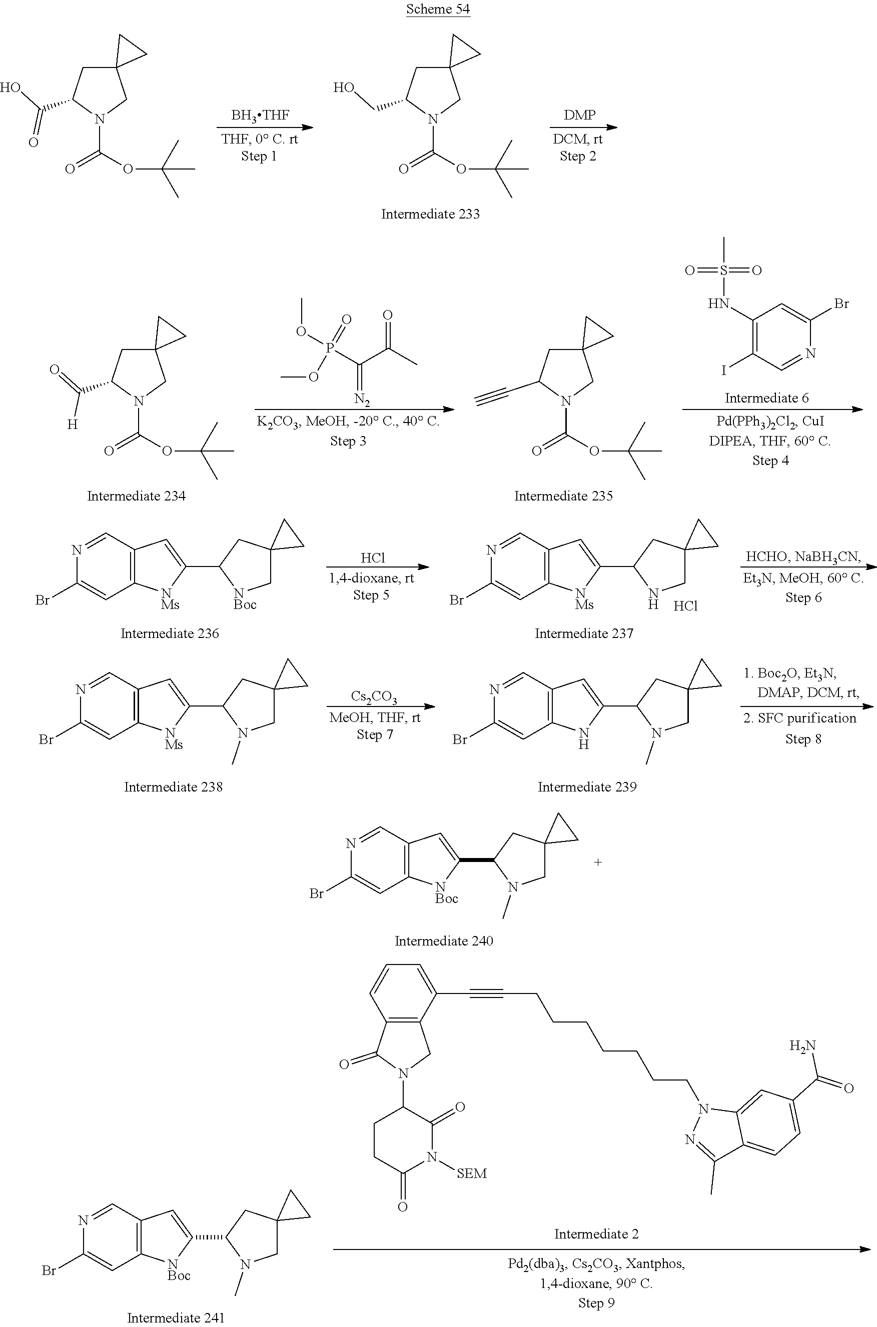

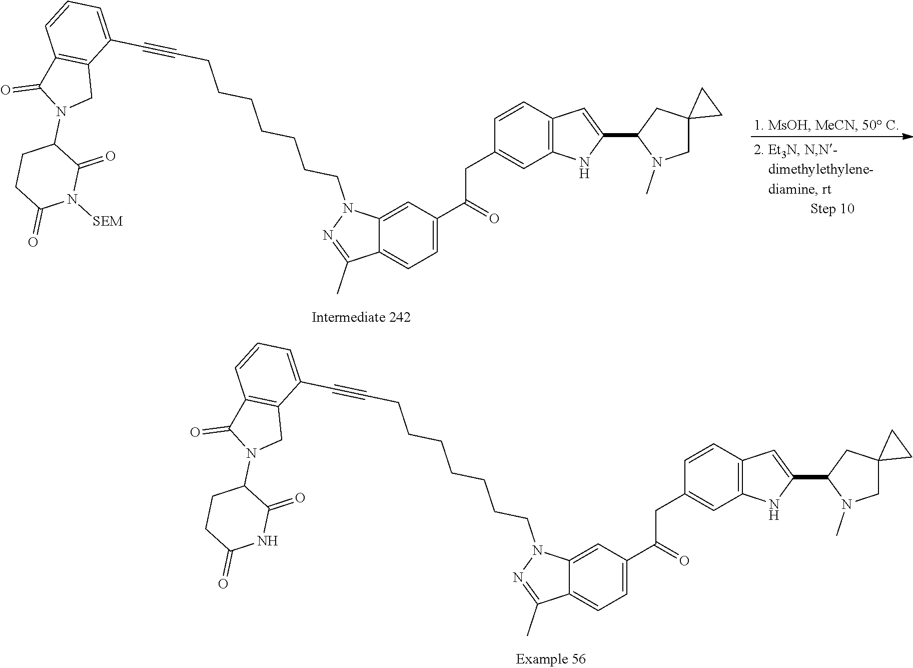

### Step 1

### Intermediate 233: tert-butyl (S)-6-(hydroxymethyl)-5-azaspiro [2.4]heptane-5-carboxylate

To a solution of (S)-5-(tert-butoxycarbonyl)-5-azaspiro[2.4]heptane-6-carboxylic acid (CAS No: 1129634-44-1, 10.0 g, 41.4 mmol) in tetrahydrofuran (150 mL) was added borane-tetrahydrofuran complex (62.2 mL, 62.2 mmol) in tetrahydrofuran (150 mL) at 0° C. and stirred at room temperature for 4 h. The reaction mixture was quenched with MeOH (25 mL) and concentrated in vacuo to afford tert-butyl (S)-6-(hydroxymethyl)-5-azaspiro[2.4]heptane-5-carboxylate (Intermediate 233, 9.00 g, 96%) as a brown liquid, which was used for the next step without further purification. ¹H NMR (400 MHz, DMSO-d₆) δ ppm 0.44-0.63 (m, 4H), 1.39 (s, 9H), 1.57-1.72 (m, 1H), 1.94-2.05 (m, 1H), 2.88-3.00 (m, 1H), 3.33-3.43 (m, 1H), 3.50-3.61 (m, 1H), 3.80-3.86 (m, 1H), 4.26-4.40 (m, 1H), 4.70 (br s, 1H). Mass spec m/z 172.5 [M-56]+.

### Step 2

### Intermediate 234: tert-butyl (S)-6-formyl-5-azaspiro [2.4]heptane-5-carboxylate

To a solution of tert-butyl (S)-6-(hydroxymethyl)-5-azaspiro[2.4]heptane-5-carboxylate (Intermediate 233, 10.0 g, 43.99 mmol) in dichloromethane (150 mL) was added Dess-Martin Periodinane (28.85 g, 65.99 mmol) at 0° C. and the reaction stirred at room temperature for 4h. The reaction mixture was diluted with water (500 mL) and extracted with dichloromethane (3×200 mL). The organic layer was separated, dried over anhydrous Na₂SO₄, filtered, and concentrated in vacuo. The crude material was purified by combi-flash column chromatography, by eluting with 20% ethyl acetate in heptane, to afford tert-butyl (S)-6-formyl-5-azaspiro[2.4]heptane-5-carboxylate (Intermediate 234, 6.9 g, 70%) as a yellow liquid. ¹H NMR (400 MHz, DMSO-d₆) δ ppm 0.40-0.48 (m, 1H), 0.51-0.64 (m, 3H), 1.41 (s, 9H), 1.75-1.87 (m, 1H), 2.03-2.14 (m, 1H), 3.19-3.26 (m, 1H), 3.28-3.36 (m, 1H), 4.16-4.25 (m, 1H), 9.48-9.54 (m, 1H).

### Step 3

### Intermediate 235: tert-butyl-6-ethynyl-5-azaspiro [2.4]heptane-5-carboxylate

To a solution of tert-butyl (S)-6-formyl-5-azaspiro [2.4]heptane-5-carboxylate (Intermediate 234, 6.40 g, 28.0 mmol) in methanol (70 mL) was added potassium carbonate (4.7 g, 34.0 mmol) followed by dimethyl (1-diazo-2-oxopropyl)phosphonate (CAS No: 90965-06-3, 8.2 g, 43.0 mmol) at −20° C. The reaction mixture was stirred at 40° C. for 3 h. The reaction mixture was diluted with water (500 mL) and extracted with dichloromethane (3×100 mL). The organic layer was separated, dried over anhydrous Na₂SO₄, filtered, and concentrated in vacuo. The crude material was purified by combi-flash column chromatography, by eluting with 5% ethyl acetate in heptane, to afford tert-butyl-6-ethynyl-5-azaspiro [2.4]heptane-5-carboxylate (Intermediate 235, 5.20 g, 75%) as a yellow solid. ¹H NMR (400 MHz, DMSO-d₆) δ ppm 0.50-0.70 (m, 4H), 1.41 (s, 9H), 1.50-1.71 (m, 1H), 2.27-2.32 (m, 1H), 2.98-3.17 (m, 2H), 3.22-3.37 (m, 1H), 4.40-4.54 (m, 1H).

### Step 4

### Intermediate 236: tert-butyl-6-(6-bromo-1-(methylsulfonyl)-1H-pyrrolo[3,2-c]pyridin-2-yl)-5-azaspiro[2.4]heptane-5-carboxylate

To a solution of N-(2-bromo-5-iodopyridin-4-yl)methanesulfonamide (Intermediate 6, 7.30 g, 19.36 mmol) in tetrahydrofuran (100 mL) was added tert-butyl-6-ethynyl-5-azaspiro[2.4]heptane-5-carboxylate (Intermediate 235, 5.14 g, 23.24 mmol) and DIPEA (27.1 mL, 155 mmol). The reaction mixture was purged with argon for 15 min followed by copper (I) iodide (0.37 g, 1.93 mmol) and Pd(PPh₃)₂Cl₂ (1.39 g, 1.94 mmol). The reaction mixture was further purged with argon for another 10 min and heated at 60° C. for 4 h. The reaction mixture was diluted with water (100 mL) and extracted with dichloromethane (3×100 mL). The organic layer was separated, dried over anhydrous Na₂SO₄, filtered, and concentrated in vacuo. The crude material was purified by combi-flash column chromatography, by eluting with 35% ethyl acetate in heptane, to afford tert-butyl-6-(6-bromo-1-(methylsulfonyl)-1H-pyrrolo[3,2-c]pyridin-2-yl)-5-azaspiro[2.4]heptane-5-carboxylate (Intermediate 236, 6.0 g, 66%) as yellow solid. ¹H NMR (401 MHz, DMSO-d₆) δ ppm 0.28-0.52 (m, 2H), 0.53-0.72 (m, 2H), 1.16-1.28 (m, 2H), 1.40 (s, 9H), 3.37 (s, 3H), 3.53-3.66 (m, 2H), 5.30-5.43 (m, 1H), 6.81 (s, 1H), 7.98 (s, 1H), 8.72 (s, 1H).

### Step 5

### Intermediate 237: 6-bromo-1-(methylsulfonyl)-2-(5-azaspiro[2.4]heptan-6-yl)-1H-pyrrolo[3,2-c]pyridine hydrochloride

To a solution of tert-butyl-6-(6-bromo-1-(methylsulfonyl)-1H-pyrrolo[3,2-c]pyridin-2-yl)-5-azaspiro[2.4]heptane-5-carboxylate (Intermediate 236, 6.5 g, 14.0 mmol) in 1,4-dioxane (60 mL) was added 4M HCl in 1,4-dioxane (70 mL). The reaction mixture was stirred at room temperature for 16 h. The reaction mixture was concentrated in vacuo to afford 6-bromo-1-(methylsulfonyl)-2-(5-azaspiro [2.4]heptan-6-yl)-1H-pyrrolo[3,2-c]pyridine hydrochloride (Intermediate 237, 5.0 g, 98%) as a yellow solid, which was used for next step without further purification. ¹H NMR (400 MHz, DMSO-d₆) δ ppm 0.68-0.76 (m, 2H), 0.78-0.92 (m, 2H), 2.26-2.35 (m, 2H), 3.07-3.16 (m, 1H), 3.25-3.35 (m, 1H), 3.82 (s, 3H), 5.35-5.46 (m, 1H), 7.46 (s, 1H), 8.03 (s, 1H), 8.84 (s, 1H), 9.68 (br s, 1H), 9.54 (br s, 1H). Mass spec m/z 370.1 [M+H]⁺.

### Step 6

### Intermediate 238: 6-bromo-2-(5-methyl-5-azaspiro[2.4]heptan-6-yl)-1-(methylsulfonyl)-1H-pyrrolo[3,2-c]pyridine

To a cooled (0° C.) solution of 6-bromo-1-(methylsulfonyl)-2-(5-azaspiro[2.4]heptan-6-yl)-1H-pyrrolo[3,2-c]pyridine hydrochloride (Intermediate 237, 6.5 g, 18.0 mmol) in methanol (70 mL) was added triethylamine (12.4 mL, 88.5 mmol) and formaldehyde (1.95 mL, 52.9 mmol). After stirring for 5 min, sodium cyanoborohydride (2.62 g, 35.4 mmol) was added and the reaction mixture was heated at 60° C. for 4 h. The reaction mixture was diluted with water (500 mL) and extracted with dichloromethane (3×250 mL). The organic layer was separated, dried over anhydrous Na₂SO₄, filtered, and concentrated in vacuo. The crude material was purified by combi-flash column chromatography, by eluting with 35% ethyl acetate in heptane, to afford 6-bromo-2-(5-methyl-5-azaspiro [2.4]heptan-6-yl)-1-(methylsulfonyl)-1H-pyrrolo[3,2-c]pyridine (Intermediate 238, 4.8 g, 71%) as a yellow solid. ¹H NMR (400 MHz, DMSO-d₆) δ ppm 0.48-0.54 (m, 1H), 0.56-0.58 (m, 2H), 0.59-0.72 (m, 1H), 1.68-1.73 (m, 1H), 2.33 (s, 3H), 2.40-2.46 (m, 1H), 2.60-2.66 (m, 1H), 2.78-2.82 (m, 1H), 3.56 (s, 3H), 4.02-4.08 (m, 1H), 6.99 (s, 1H), 7.96 (s, 1H), 8.70 (s, 1H).

### Step 7

### Intermediate 239: 6-bromo-2-(5-methyl-5-azaspiro[2.4]heptan-6-yl)-1H-pyrrolo[3,2-c]pyridine

To a solution of 6-bromo-2-(5-methyl-5-azaspiro[2.4]heptan-6-yl)-1-(methylsulfonyl)-1H-pyrrolo [3,2-c]pyridine (Intermediate 238, 4.8 g, 12.0 mmol) in methanol (50 mL) and tetrahydrofuran (50 mL) was added cesium carbonate (8.1 g, 25.0 mmol). The reaction mixture was stirred at room temperature for 4 h. The reaction mixture was quenched with water (100 mL) and extracted ethyl acetate (2×200 mL). The organic layer was separated, dried over anhydrous Na₂SO₄, filtered, and concentrated in vacuo to afford (6-bromo-2-(5-methyl-5-azaspiro[2.4]heptan-6-yl)-1H-pyrrolo[3,2-c]pyridine (Intermediate 239, 2.8 g, 73%) as a yellow solid, which was used for next step without further purification. ¹H NMR (400 MHz, DMSO-d₆) δ ppm 0.48-0.56 (m, 2H), 0.58-0.72 (m, 2H), 1.94-2.07 (m, 2H), 2.14 (s, 3H), 2.84-2.92 (m, 1H), 3.55-3.59 (m, 1H), 3.62-3.70 (m, 1H), 6.48 (s, 1H), 7.44 (s, 1H), 8.49 (s, 1H), 11.68 (br s, 1H). Mass spec m/z 306.2 [M+H]⁺.

### Step 8

### Intermediate 240: tert-butyl-(rel-R)-6-bromo-2-(5-methyl-5-azaspiro[2.4]heptan-6-yl)-1H-pyrrolo[3,2-c]pyridine-1-carboxylate and Intermediate 241: tert-butyl-(rel-S)-6-bromo-2-(5-methyl-5-azaspiro[2.4]heptan-6-yl)-1H-pyrrolo[3,2-c]pyridine-1-carboxylate

To a cooled (0° C.) solution of 6-bromo-2-(5-methyl-5-azaspiro[2.4]heptan-6-yl)-1H-pyrrolo [3,2-c]pyridine (Intermediate 239, 5.6 g, 18.0 mmol) in dichloromethane (60 mL) was added triethylamine (10.0 mL, 73.0 mmol) and di-tert-butyl dicarbonate (8.1 g, 37.0 mmol) followed by 4-dimethylaminopyridine (0.45 g, 3.7 mmol). The reaction mixture was stirred at room temperature for 4 h. The reaction mixture was diluted with water (300 mL) and extracted with dichloromethane (3×200 mL). The organic layer was separated, dried over anhydrous Na₂SO₄, filtered, and concentrated in vacuo. The resulting material was purified by combi-flash column chromatography, by eluting with 45% ethyl acetate in heptane to afford crude product. This material was further purified by SFC chiral purification (Column: Chiralpak IK 250*30 mm, 5 μm. Isocratic elution 25% Mobile Phase (A): CO₂, Mobile Phase (B): MeCN+isopropylalcohol) to afford tert-butyl-(rel-S)-6-bromo-2-(5-methyl-5-azaspiro[2.4]heptan-6-yl)-1H-pyrrolo[3,2-c]pyridine-1-carboxylate (Intermediate 241, 1.9 g, 26%) and tert-butyl-(rel-R)-6-bromo-2-(5-methyl-5-azaspiro[2.4]heptan-6-yl)-1H-pyrrolo[3,2-c]pyridine-1-carboxylate (Intermediate 240, 1.40 g, 19%) as a white solid.

### Intermediate 241 Exhibited

1st eluting peak, retention time: 7.67 mins.

¹H NMR (400 MHz, DMSO-d₆) δ ppm 0.34-0.42 (m, 1H), 0.46-0.64 (m, 3H), 1.56-1.61 (m, 1H), 1.64 (s, 9H), 2.33 (s, 3H), 2.40-2.47 (m, 1H), 2.61-2.66 (m, 1H), 2.78-2.80 (m, 1H), 4.10-4.16 (m, 1H), 6.89 (s, 1H), 8.04 (s, 1H), 8.63 (s, 1H). Mass spec m/z 408.10 [M+H]⁺.

### Intermediate 240 Exhibited

2^{nd} eluting peak, retention time: 9.99 mins.

¹H NMR (400 MHz, DMSO-d₆) δ ppm 0.34-0.42 (m, 1H), 0.46-0.64 (m, 3H), 1.56-1.61 (m, 1H), 1.64 (s, 9H), 2.33 (s, 3H), 2.40-2.47 (m, 1H), 2.61-2.66 (m, 1H), 2.78-2.80 (m, 1H), 4.10-4.16 (m, 1H), 6.89 (s, 1H), 8.04 (s, 1H), 8.63 (s, 1H). Mass spec m/z 408.10 [M+H]⁺.

### Step 9

### Intermediate 242: tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((rel-R)-5-methyl-5-azaspiro[2.4]heptan-6-yl)-1H-pyrrolo[3,2-c]pyridine-1-carboxylate

To a mixture of tert-butyl (rel-R)-6-bromo-2-(5-methyl-5-azaspiro [2.4]heptan-6-yl)-1H-pyrrolo [3,2-c]pyridine-1-carboxylate (Intermediate 240, 0.4 g, 0.98 mmol) and 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 2, 0.52 g, 0.78 mmol) in 1,4-dioxane (15 mL) was added cesium carbonate (0.96 g, 2.95 mmol) at room temperature. The reaction mixture was purged with argon for 15 min. then Pd₂(dba)₃ (0.13 g, 0.14 mmol) and Xantphos (0.17 g, 0.29 mmol) were added. The reaction mixture was further purged with argon for another 10 min and heated at 90° C. for 2 h. The reaction mixture was filtered through a celite bed and washed with ethyl acetate (100 mL). The filtrate was concentrated in vacuo to obtain the crude compound. The crude material was purified by combi-flash column chromatography, by eluting with 90% ethyl acetate in heptane, to afford tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((rel-R)-5-methyl-5-azaspiro[2.4]heptan-6-yl)-1H-pyrrolo[3,2-c]pyridine-1-carboxylate (Intermediate 242, 0.58 g, 59%) as a yellow solid. ¹H NMR (400 MHz, DMSO-d₆) δ ppm −0.06 (s, 9H), 0.40-0.44 (m, 1H), 0.48-0.65 (m, 3H), 0.75-0.86 (m, 2H), 1.24-1.44 (m, 6H), 1.51-1.56 (m, 2H), 1.60-1.64 (m, 2H), 1.70 (s, 9H), 1.81-1.93 (m, 2H), 2.19 (s, 3H), 2.34 (s, 3H), 2.42-2.46 (m, 2H), 2.61-2.69 (m, 2H), 2.73-2.83 (m, 2H), 2.99-3.11 (m, 2H), 3.47-3.54 (m, 2H), 4.13-4.26 (m, 2H), 4.37-4.48 (m, 3H), 4.97-5.10 (m, 2H), 5.24-5.28 (m, 1H), 6.85 (s, 1H), 7.47-7.53 (m, 1H), 7.58-7.62 (m, 1H), 7.68-7.74 (m, 2H), 7.76-7.81 (m, 1H), 8.42 (s, 1H), 8.62 (s, 1H), 8.97 (s, 1H), 10.74 (s, 1H). Mass spec m/z 994.7 [M+H]⁺.

### Step 10

### Example 56: 1-(9-(2-(2,6-dioxopiperidin-3-yl)-1-oxoisoindolin-4-yl)non-8-yn-1-yl)-3-methyl-N-(2-((rel-R)-5-methyl-5-azaspiro[2.4]heptan-6-yl)-1H-pyrrolo[3,2-c]pyridin-6-yl)-1H-indazole-6-carboxamide

To a solution of tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-((rel-R)-5-methyl-5-azaspiro[2.4]heptan-6-yl)-1H-pyrrolo[3,2-c]pyridine-1-carboxylate (Intermediate 242, 0.58 g, 0.58 mmol) in acetonitrile (15 mL) was added methanesulfonic acid (0.58 g, 5.9 mmol) at room temperature and the reaction heated at 50° C. for 2h. To the reaction mixture N, N′-dimethylethylenediamine (0.29 g, 2.9 mmol) and triethylamine (1.64 mL, 11.7 mmol) were added. The reaction mixture was stirred at room temperature for 3 h. The reaction mixture was concentrated in vacuo. The crude compound was quenched with water (50 mL) and the resultant precipitate collected by filtration and dried in vacuo. The crude material was purified by preparative HPLC (Method A) to afford 1-(9-(2-(2,6-dioxopiperidin-3-yl)-1-oxoisoindolin-4-yl)non-8-yn-1-yl)-3-methyl-N-(2-((rel-R)-5-methyl-5-azaspiro[2.4]heptan-6-yl)-1H-pyrrolo[3,2-c]pyridin-6-yl)-1H-indazole-6-carboxamide (Example 56, 0.19 g, 43%) as an off white solid. ¹H NMR (400 MHz, DMSO-d₆) δ ppm 0.50-0.73 (m, 4H), 1.25-1.45 (m, 6H), 1.49-1.60 (m, 2H), 1.82-1.91 (m, 2H), 1.95-2.10 (m, 3H), 2.16 (s, 3H), 2.40-2.48 (m, 4H), 2.52 (s, 3H), 2.54-2.61 (m, 1H), 2.84-2.96 (m, 2H), 3.59-3.63 (m, 1H), 4.22-4.35 (m, 1H), 4.36-4.46 (m, 3H), 5.11-5.16 (m, 1H), 6.44 (s, 1H), 7.46-7.52 (m, 1H), 7.58-7.63 (m, 1H), 7.68-7.80 (m, 3H), 8.25 (s, 1H), 8.43 (s, 1H), 8.52 (s, 1H), 10.52 (s, 1H), 11.00 (s, 1H), 11.47 (s, 1H). Mass spec m/z 765.0 [M+H]⁺.

### Example 57 was Synthesized Following Scheme 55

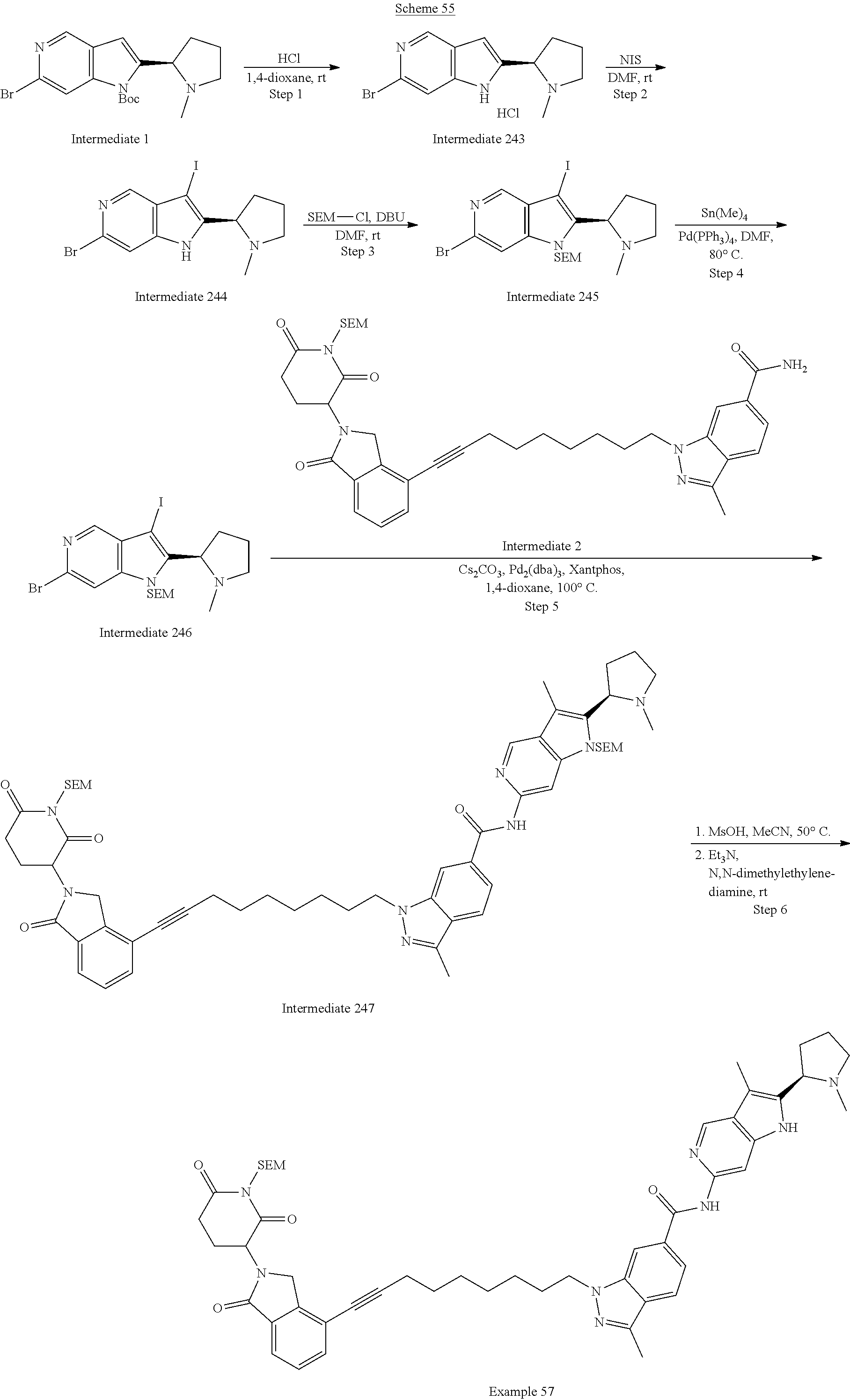

### Step 1

### Intermediate 243: (R)-6-bromo-2-(1-methylpyrrolidin-2-yl)-1H-pyrrolo[3,2-c]pyridine hydrochloride

To a solution of tert-butyl (R)-6-bromo-2-(1-methylpyrrolidin-2-yl)-1H-pyrrolo[3,2-c]pyridine-1-carboxylate (Intermediate 1, 10.0 g, 26.3 mmol) in 1,4-dioxane (100 mL) was added 4M HCl in 1,4-dioxane (100 mL) at 0° C. The reaction mixture was stirred at room temperature for 16 h. The reaction mixture was concentrated in vacuo to obtain the crude compound. The crude material was washed with ether (100 mL) to afford (R)-6-bromo-2-(1-methylpyrrolidin-2-yl)-1H-pyrrolo[3,2-c]pyridine hydrochloride (Intermediate 243, 8.00 g) as an off white solid, which was used for the next step without further purification. Mass spec: m/z: 280.5 [M+H]⁺.

### Step 2

### Intermediate 244: (R)-6-bromo-3-iodo-2-(1-methylpyrrolidin-2-yl)-1H-pyrrolo [3,2-c]pyridine

To a solution of (R)-6-bromo-2-(1-methylpyrrolidin-2-yl)-1H-pyrrolo[3,2-c]pyridine hydrochloride (Intermediate 243, 2.50 g, 8.9 mmol) in dimethylformamide (20 mL) was added N-iodosuccinimide (4.2 g, 18 mmol). The reaction mixture was stirred at room temperature for 16 h. The reaction mixture was concentrated in vacuo. The crude compound was quenched with water (50 mL) and the resultant precipitate collected by filtration and dried in vacuo to afford (R)-6-bromo-3-iodo-2-(1-methylpyrrolidin-2-yl)-1H-pyrrolo [3,2-c]pyridine (Intermediate 244, 1.50 g, 41%) as a brown solid, which was used for the next step without further purification. ¹H NMR (400 MHz, DMSO-d₆) δ ppm 1.69-1.97 (m, 3H), 2.15 (s, 3H), 2.17-2.20 (m, 1H), 2.29-2.35 (m, 1H), 3.16-3.20 (m, 1H), 3.46-3.50 (m, 1H), 7.45 (s, 1H), 8.26 (s, 1H), 11.96 (br s, 1H). Mass spec m/z 406.0 [M+H]⁺.

### Step 3

### Intermediate 245: (R)-6-bromo-3-iodo-2-(1-methylpyrrolidin-2-yl)-1-((2-(trimethylsilyl) ethoxy) methyl)-1H-pyrrolo [3,2-c]pyridine

To a solution of (R)-6-bromo-3-iodo-2-(1-methylpyrrolidin-2-yl)-1H-pyrrolo [3,2-c]pyridine (Intermediate 244, 1.40 g, 3.4 mmol) in dimethylformamide (15 mL) was added DBU (1.9 g, 12 mmol) at 0° C. followed by SEM-Cl (2.2 mL, 12 mmol) under nitrogen atmosphere. The reaction mixture was stirred at room temperature for 16 h then quenched with ice cold water (100 mL) and extracted with ethyl acetate (2×100 mL). The organic layer was separated, washed with brine solution (100 mL), dried over anhydrous Na₂SO₄, filtered and concentrated in vacuo. The crude material was purified by combi-flash chromatography, by eluting with 15% ethyl acetate in heptane, to afford (R)-6-bromo-3-iodo-2-(1-methylpyrrolidin-2-yl)-1-((2-(trimethylsilyl) ethoxy) methyl)-1H-pyrrolo [3,2-c]pyridine (Intermediate 245, 1.30 g, 70%) as a colourless liquid. ¹H NMR (400 MHz, DMSO-d₆) δ ppm −0.10 (s, 9H), 0.72-0.88 (m, 2H), 1.78-1.96 (m, 2H), 1.98-2.04 (m, 1H), 2.07 (s, 3H), 2.09-2.14 (m, 1H), 2.16-2.24 (m, 1H), 3.13-3.16 (m, 1H), 3.42-3.50 (m, 1H), 3.52-3.60 (m, 1H), 3.65-3.69 (m, 1H), 5.65-7.68 (m, 1H), 5.92-5.96 (m, 1H), 7.82 (s, 1H), 8.26 (s, 1H). Mass spec m/z 536.0 [M+H]⁺.

### Step 4

### Intermediate 246: (R)-6-bromo-3-methyl-2-(1-methylpyrrolidin-2-yl)-1-((2-(trimethylsilyl) ethoxy) methyl)-1H-pyrrolo [3,2-c]pyridine

To a solution of (R)-6-bromo-3-iodo-2-(1-methylpyrrolidin-2-yl)-1-((2-(trimethylsilyl) ethoxy) methyl)-1H-pyrrolo [3,2-c]pyridine (Intermediate 245, 1.00 g, 1.9 mmol) in dimethylformamide (10 mL) was added tetramethyl tin (1.1 g, 5.6 mmol) and the mixture purged with argon for 15 min. To the reaction mixture Pd(PPh₃)₄ (0.22 g, 0.19 mmol) was added and the mixture purged with argon for 10 min. The reaction mixture was heated at 80° C. for 3h. The reaction mixture was poured into ice cold water (30 mL) and extracted with ethyl acetate (3×50 mL). The organic layer was separated, dried over anhydrous Na₂SO₄, filtered, and concentrated in vacuo. The crude material was purified by combi-flash chromatography, by eluting with 40% ethyl acetate in heptane, to afford (R)-6-bromo-3-methyl-2-(1-methylpyrrolidin-2-yl)-1-((2-(trimethylsilyl) ethoxy) methyl)-1H-pyrrolo [3,2-c]pyridine (Intermediate 246, 0.40 g, 51%) as yellow sticky solid. ¹H NMR (400 MHz, DMSO-d₆) δ ppm −0.07 (s, 9H), 0.74-0.89 (m, 2H), 1.77-2.04 (m, 3H), 2.09 (s, 3H), 2.13-2.22 (m, 1H), 2.31 (s, 3H), 3.13-3.17 (m, 1H), 3.40-3.59 (m, 4H), 5.58-5.62 (m, 1H), 5.80-5.82 (m, 1H), 7.77 (s, 1H), 8.53 (s, 1H). Mass spec m/z 424.20 [M+H]⁺.

### Step 5

### Intermediate 247: 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-N-(3-methyl-2-((R)-1-methylpyrrolidin-2-yl)-1-((2-(trimethylsilyl)ethoxy)methyl)-1H-pyrrolo[3,2-c]pyridin-6-yl)-1H-indazole-6-carboxamide

To a solution of 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 2, 0.42 g, 0.72 mmol) in 1,4-dioxane (15 mL) was added (R)-6-bromo-3-methyl-2-(1-methylpyrrolidin-2-yl)-1-((2-(trimethylsilyl)ethoxy)methyl)-1H-pyrrolo[3,2-c]pyridine (Intermediate 246, 0.40 g, 0.91 mmol) followed by cesium carbonate (0.89 g, 2.72 mmol) at room temperature. The reaction mixture was purged with argon for 15 min followed by Pd₂(dba)₃ (0.13 g, 0.13 mmol) and Xantphos (0.16 g, 0.27 mmol). The reaction mixture was further purged with argon for 10 min and heated at 100° C. for 2 h. The reaction mixture was concentrated in vacuo to obtain the crude compound. The crude material was purified by combi-flash chromatography, by eluting with 80% ethyl acetate in heptane, to afford 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-N-(3-methyl-2-((R)-1-methylpyrrolidin-2-yl)-1-((2-(trimethylsilyl)ethoxy)methyl)-1H-pyrrolo[3,2-c]pyridin-6-yl)-1H-indazole-6-carboxamide (Intermediate 247, 0.42 g, 46%) as a yellow solid. ¹H NMR (400 MHz, DMSO-d₆) δ ppm −0.06 (s, 9H), −0.05 (s, 9H), 0.76-0.96 (m, 4H), 1.22-1.44 (m, 6H), 1.48-1.59 (m, 2H), 1.80-1.94 (m, 5H), 2.00-2.09 (m, 2H), 2.12 (s, 3H), 2.15-2.23 (m, 2H), 2.33 (s, 3H), 2.41-2.46 (m, 3H), 2.74-2.80 (m, 2H), 3.02-3.10 (m, 1H), 3.15-3.20 (m, 1H), 3.45-3.64 (m, 6H), 4.22-4.26 (m, 1H), 4.37-4.47 (m, 3H), 4.97-5.09 (m, 2H), 5.24-5.30 (m, 1H), 5.52-5.56 (m, 1H), 5.84-5.88 (m, 1H), 7.46-7.52 (m, 1H), 7.58-7.62 (m, 1H), 7.68-7.82 (m, 3H), 8.41 (s, 1H), 8.45 (s, 1H), 8.55 (s, 1H), 10.65 (s, 1H). Mass spec m/z 1013.64 [M+H]⁺.

### Step 6

### Example 57: 1-(9-(2-(2,6-dioxopiperidin-3-yl)-1-oxoisoindolin-4-yl)non-8-yn-1-yl)-3-methyl-N-(3-methyl-2-((R)-1-methylpyrrolidin-2-yl)-1H-pyrrolo[3,2-c]pyridin-6-yl)-1H-indazole-6-carboxamide

To a solution of 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-N-(3-methyl-2-((R)-1-methylpyrrolidin-2-yl)-1-((2-(trimethylsilyl)ethoxy)methyl)-1H-pyrrolo[3,2-c]pyridin-6-yl)-1H-indazole-6-carboxamide (Intermediate 247, 0.38 g, 0.37 mmol) in acetonitrile (10 mL) was added methanesulfonic acid (0.49 mL, 7.49 mmol) at room temperature and the reaction mixture was heated at 50° C. for 2h. After cooling to room temperature, N,N′-dimethylethylenediamine (0.45 mL, 3.75 mmol) followed by triethylamine (2.10 mL, 14.99 mmol) were added and the reaction stirred for 3h. The reaction mixture was poured into ice cold water (20 mL) and the resultant precipitate collected by filtration and dried in vacuo to obtain the crude compound. The crude material was purified by preparative HPLC (Method A) to afford 1-(9-(2-(2,6-dioxopiperidin-3-yl)-1-oxoisoindolin-4-yl)non-8-yn-1-yl)-3-methyl-N-(3-methyl-2-((R)-1-methylpyrrolidin-2-yl)-1H-pyrrolo[3,2-c]pyridin-6-yl)-1H-indazole-6-carboxamide (Example 57, 0.12 g, 43%) as an off white solid. ¹H NMR (400 MHz, DMSO-d) δ ppm 1.27-1.44 (m, 6H), 1.47-1.60 (m, 2H), 1.78-2.02 (m, 6H), 2.07-2.11 (m, 1H), 2.13 (s, 3H), 2.23-2.26 (m, 1H), 2.27 (s, 3H), 2.42-2.47 (m, 3H), 2.49 (s, 3H), 2.54-2.62 (m, 1H), 2.85-2.94 (m, 1H), 3.14-3.18 (m, 1H), 3.39-3.43 (m, 1H), 4.25-4.33 (m, 1H), 4.37-4.46 (m, 3H), 5.10-5.15 (m, 1H), 7.44-7.53 (m, 1H), 7.58-7.60 (m, 1H), 7.67-7.80 (m, 3H), 8.20 (s, 1H), 8.43 (s, 1H), 8.49 (s, 1H), 10.50 (s, 1H), 11.00 (s, 1H), 11.06 (s, 1H). Mass spec m/z 753.2 [M+H]⁺.

### Example 58 was Synthesized Following Scheme 56

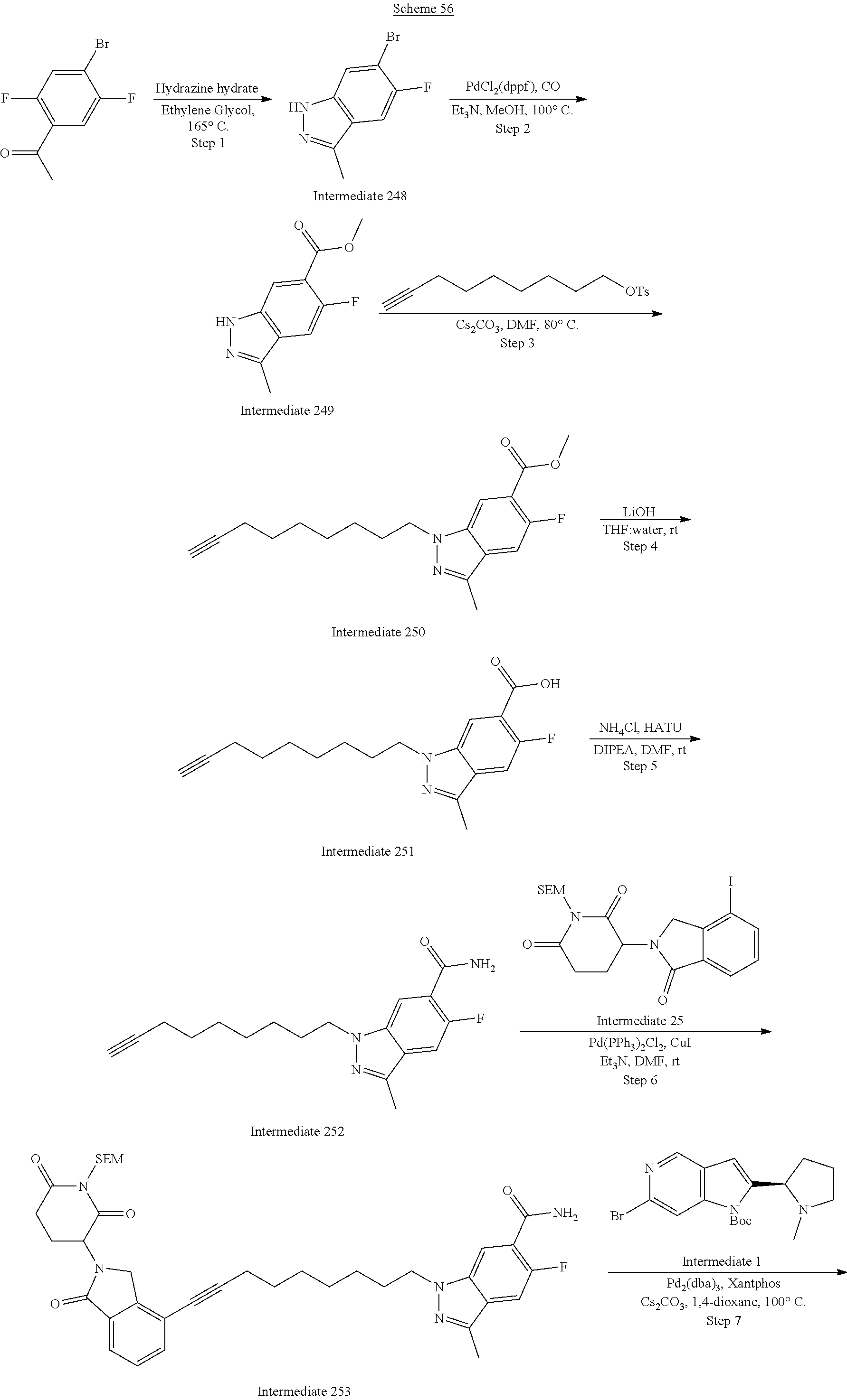

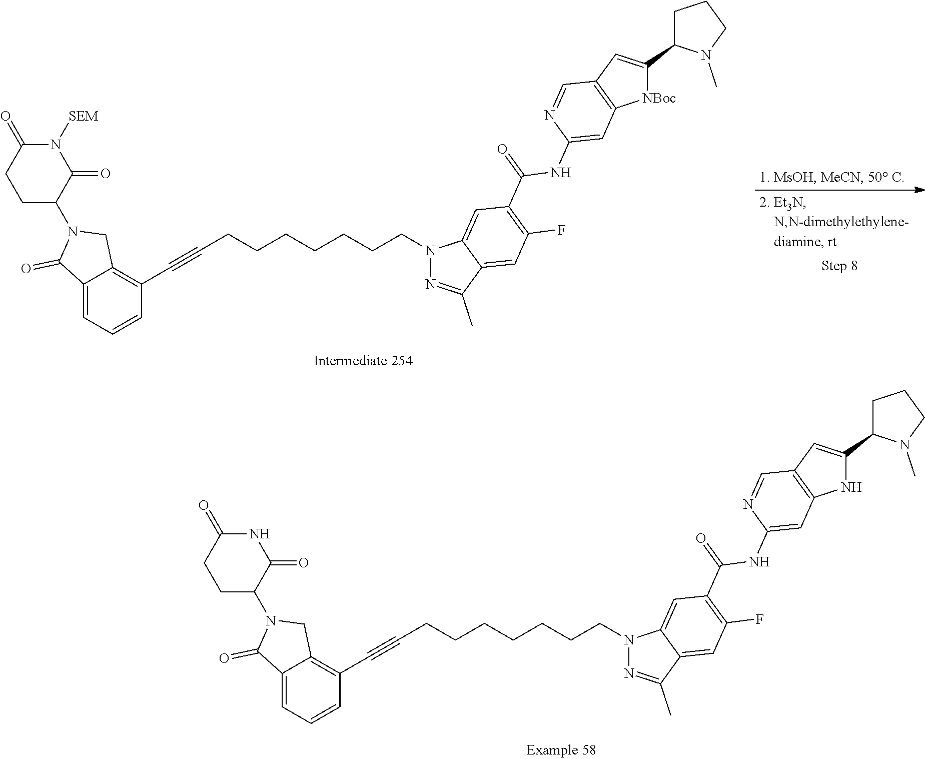 [SMILES: Cc1nn(CCCCCCCC#Cc2cccc3c2CN(C2CCC(=O)N(COCC[Si](C)(C)C)C2=O)C3=O)c2cc(C(=O)Nc3cc4c(cn3)cc([C@H]3CCCN3C)n4C(=O)OC(C)(C)C)c(F)cc12 ; Cc1nn(CCCCCCCC#Cc2cccc3c2CN(C2CCC(=O)NC2=O)C3=O)c2cc(C(=O)Nc3cc4[nH]c([C@H]5CCCN5C)cc4cn3)c(F)cc12]

### Step 1

### Intermediate 248: 6-bromo-5-fluoro-3-methyl-1H-indazole

To a solution of 1-(4-bromo-2,5-difluorophenyl)ethan-1-one (CAS No: 123942-11-0, 5.00 g, 21.27 mmol) in ethylene glycol (50 mL, 892.0 mmol) was added hydrazine monohydrate (1.69 mL, 53.18 mmol) at room temperature. The reaction mixture was heated at 165° C. for 16 h. The reaction mixture was quenched with water (1000 mL) and the resultant precipitate collected by filtration. The obtained solid was washed with water (3×250 mL) and n-pentane (3×60 mL) and then dried in vacuo to afford 6-bromo-5-fluoro-3-methyl-1H-indazole (Intermediate 248, 2.50 g, 51%) as an off white solid, which was used for the next step without further purification. 25 ¹H NMR (400 MHz, DMSO-d) δ ppm 2.46 (s, 3H), 7.72 (d, J=8.93 Hz, 1H), 7.82 (d, J=5.62 Hz, 1H), 12.83 (br s, 1H). Mass spec m/z 231.3 [M+H]⁺.

### Step 2

### Intermediate 249: methyl 5-fluoro-3-methyl-1H-indazole-6-carboxylate

To a solution of 6-bromo-5-fluoro-3-methyl-1H-indazole (Intermediate 248, 2.50 g, 11.0 mmol) in methanol (40 mL) was added triethylamine (4.6 mL, 33.0 mmol) followed by PdCl₂(dppf) (0.81 g, 1.1 mmol) at room temperature. The reaction mixture was stirred under CO atmosphere (100 psi) at 100° C. for 16 h. The reaction mixture was filtered through a celite bed and concentrated in vacuo. The crude material was purified by combi-flash chromatography, by eluting with 30% ethyl acetate in heptane, to afford methyl 5-fluoro-3-methyl-1H-indazole-6-carboxylate (Intermediate 249, 1.60 g, 70%) as an off white solid. ¹H NMR (400 MHz, DMSO-d₆) δ ppm 2.48 (s, 3H), 3.88 (s, 3H), 7.67 (d, J=10.96 Hz, 1H), 7.98 (d, J=5.26 Hz, 1H), 13.05 (br s, 1H). Mass spec m/z 209.5 [M+H]⁺.

### Step 3

### Intermediate 250: methyl 5-fluoro-3-methyl-1-(non-8-yn-1-yl)-1H-indazole-6-carboxylate

To a solution of methyl 5-fluoro-3-methyl-1H-indazole-6-carboxylate (Intermediate 249, 1.60 g, 7.7 mmol) in dimethylformamide (13 mL) was added non-8-yn-1-yl 4-methylbenzenesulfonate (CAS 87462-64-4, 2.9 g, 10.0 mmol) followed by cesium carbonate (7.50 g, 23.0 mmol). The reaction mixture was stirred at 80° C. for 2 h. The reaction mixture was diluted with water (50 mL) and extracted with ethyl acetate (3×200 mL). The organic layer was separated, washed with brine solution (3×200 mL), dried over anhydrous Na₂SO₄, filtered, and concentrated in vacuo to obtain the crude compound as a mixture of regioisomers. The regioisomers were separated by combi-flash chromatography, by eluting with 30% ethyl acetate, to afford desired regioisomer methyl 5-fluoro-3-methyl-1-(non-8-yn-1-yl)-1H-indazole-6-carboxylate (Intermediate 250, 1.60 g, 63%) as a colourless liquid. ¹H NMR (400 MHz, DMSO-d₆) δ ppm 1.16-1.33 (m, 6H), 1.34-1.46 (m, 2H), 1.74-1.81 (m, 2H), 2.06-2.15 (m, 2H), 2.48 (s, 3H), 2.69-2.72 (m, 1H), 3.89 (s, 3H), 4.38 (t, J=7.02 Hz, 2H), 7.66 (d, J=10.96 Hz, 1H), 8.16 (d, J=5.70 Hz, 1H). Mass spec m/z 331.3 [M+H]⁺.

### Step 4

### Intermediate 251: 5-fluoro-3-methyl-1-(non-8-yn-1-yl)-1H-indazole-6-carboxylic acid

To a solution of methyl 5-fluoro-3-methyl-1-(non-8-yn-1-yl)-1H-indazole-6-carboxylate (Intermediate 250, 1.5 g, 4.5 mmol) in methanol (11 mL) and tetrahydrofuran (11 mL) was added lithium hydroxide (0.44 g, 18.0 mmol) in water (5 mL) at room temperature. The reaction mixture was stirred at room temperature for 16 h. The reaction mixture was concentrated in vacuo. The reaction mixture was diluted with water (25 mL) and the aqueous layer was acidified by adjusted to pH~4 with 0.5 N HCl. The resultant precipitate collected by filtration and dried in vacuo to afford 5-fluoro-3-methyl-1-(non-8-yn-1-yl)-1H-indazole-6-carboxylic acid (Intermediate 251, 0.90 g, 63%) as a white solid, which was used for the next step without further purification. ¹H NMR (400 MHz, DMSO-d₆) δ ppm 1.17-1.32 (m, 6H), 1.34-1.44 (m, 2H), 1.74-1.81 (m, 2H), 2.08-2.13 (m, 2H), 2.47 (s, 3H), 2.68-2.70 (m, 1H), 4.37 (t, J=7.00 Hz, 2H), 7.60 (d, J=10.88 Hz, 1H), 8.11 (d, J=5.63 Hz, 1H), 13.32 (br s, 1H). Mass spec m/z 317.1 [M+H]⁺.

### Step 5

### Intermediate 252: 5-fluoro-3-methyl-1-(non-8-yn-1-yl)-1H-indazole-6-carboxamide

To a solution of 5-fluoro-3-methyl-1-(non-8-yn-1-yl)-1H-indazole-6-carboxylic acid (Intermediate 251, 1.1 g, 3.5 mmol) in dimethylformamide (11 mL) were added HATU (2.0 g, 5.2 mmol) and DIPEA (1.8 mL, 10.0 mmol) at room temperature and stirred for 20 min. Then ammonium chloride (0.93 g, 17.0 mmol) was added and the reaction mixture was stirred at room temperature for 16 h. The reaction mixture was quenched with water (200 mL) to obtain a precipitate which was filtered and dried in vacuo to afford 5-fluoro-3-methyl-1-(non-8-yn-1-yl)-1H-indazole-6-carboxamide (Intermediate 252, 0.85 g, 74%) as a white solid, which was used in the next step without further purification. ¹H NMR (400 MHz, DMSO-d₆) δ ppm 1.19-1.31 (m, 6H), 1.35-1.46 (m, 2H), 1.75-1.81 (m, 2H), 2.09-2.14 (m, 2H), 2.46 (s, 3H), 2.70 (s, 1H), 4.33 (t, J=6.80 Hz, 2H), 7.57 (d, J=10.40 Hz, 1H), 7.66 (br s, 1H), 7.75 (br s, 1H), 7.87 (d, J=6.00 Hz, 1H). Mass spec m/z 317.1 [M+H]⁺.

### Step 6

### Intermediate 253: 1-(9-(2-(2,6-dioxo-1-((2-(trimethylsilyl) ethoxy) methyl) piperidin-3-yl)-1-oxoisoindolin-4-yl) non-8-yn-1-yl)-5-fluoro-3-methyl-1H-indazole-6-carboxamide

To a mixture of 5-fluoro-3-methyl-1-(non-8-yn-1-yl)-1H-indazole-6-carboxamide (Intermediate 252, 0.40 g, 1.26 mmol) and 3-(4-iodo-1-oxoisoindolin-2-yl)-1-((2-(trimethylsilyl)ethoxy)methyl)piperidine-2,6-dione (Intermediate 25, 0.63 g, 1.26 mmol) in dimethylformamide (5 mL) was added triethylamine (6.40 mL, 45.66 mmol). The reaction mixture was purged with argon gas for 15 min. To the reaction mixture PdCl₂(PPh₃)₂ (0.08 g, 0.12 mmol) and copper (I) iodide (0.02 g, 0.12 mmol) were added. Then the reaction mixture was purged with argon gas for another 10 min and stirred at room temperature for 2 h. The reaction mixture was diluted with water (200 mL) and extracted with ethyl acetate (3×150 mL). The organic layer was separated, dried over anhydrous Na₂SO₄, filtered, and concentrated in vacuo. The crude material was purified by combi-flash chromatography, by eluting with 80% ethyl acetate in heptane, to afford 1-(9-(2-(2,6-dioxo-1-((2-(trimethylsilyl) ethoxy) methyl) piperidin-3-yl)-1-oxoisoindolin-4-yl) non-8-yn-1-yl)-5-fluoro-3-methyl-1H-indazole-6-carboxamide (Intermediate 253, 0.70 g, 81%) as a yellow solid. ¹H NMR (400 MHz, DMSO-d₆) δ ppm −0.04 (s, 9H), 0.77-0.87 (m, 2H), 1.17-1.44 (m, 6H), 1.50-1.57 (m, 2H), 1.74-1.82 (m, 2H), 2.01-2.09 (m, 1H), 2.40-2.42 (m, 1H), 2.45 (s, 3H), 2.46-2.48 (m, 2H), 2.75-2.82 (m, 1H), 3.01-3.12 (m, 1H), 3.46-3.57 (m, 2H), 4.22-4.37 (m, 3H), 4.43-4.47 (m, 1H), 4.99-5.10 (m, 2H), 5.25-5.29 (m, 1H), 7.49-7.63 (m, 3H), 7.66 (br s, 1H), 7.70-7.72 (m, 1H), 7.74 (br s, 1H), 7.88 (d, J=5.26 Hz, 1H). Mass spec m/z 686.0 [M+H]⁺.

### Step 7

### Intermediate 254: tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-5-fluoro-3-methyl-1H-indazole-6-carboxamido)-2-((R)-1-methylpyrrolidin-2-yl)-1H-pyrrolo[3,2-c]pyridine-1-carboxylate

To a solution of 1-(9-(2-(2,6-dioxo-1-((2-(trimethylsilyl) ethoxy) methyl) piperidin-3-yl)-1-oxoisoindolin-4-yl) non-8-yn-1-yl)-5-fluoro-3-methyl-1H-indazole-6-carboxamide (Intermediate 253, 0.65 g, 0.94 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 1, 0.45 g, 1.18 mmol) followed by cesium carbonate (1.15 g, 3.55 mmol) at room temperature. The reaction mixture was purged with argon for 15 min. To the reaction mixture Pd₂(dba)₃ (0.16 g, 0.17 mmol) and Xantphos (0.21 g, 0.35 mmol) were added. Then the reaction mixture was purged with argon for 10 min and stirred at 100° C. for 2 h. The reaction mixture was filtered through a celite bed and washed with ethyl acetate (50 mL). The filtrate was concentrated in vacuo. The crude material was purified by combi-flash column chromatography, by eluting with 100% ethyl acetate in heptane, to afford tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-5-fluoro-3-methyl-1H-indazole-6-carboxamido)-2-((R)-1-methylpyrrolidin-2-yl)-1H-pyrrolo[3,2-c]pyridine-1-carboxylate (Intermediate 254, 0.48 g, 41%) as yellow solid. ¹H NMR (400 MHz, DMSO-d₆) δ ppm −0.05 (s, 9H), 0.75-0.87 (m, 2H), 1.22-1.44 (m, 6H), 1.48-1.63 (m, 4H), 1.69 (s, 9H), 1.71-1.87 (m, 4H), 1.95-2.09 (m, 2H), 2.36 (s, 3H), 2.47 (s, 3H), 2.73-2.79 (m, 1H), 2.98-3.19 (m, 3H), 3.41-3.57 (m, 3H), 3.87-3.97 (m, 1H), 4.22-4.46 (m, 5H), 4.97-5.10 (m, 2H), 5.24-5.29 (m, 1H), 6.75 (s, 1H), 7.46-7.54 (m, 1H), 7.57-7.65 (m, 2H), 7.70 (d, J=7.89 Hz, 1H), 7.98 (d, J=5.26 Hz, 1H), 8.57 (s, 1H), 8.95 (s, 1H), 10.78 (br s, 1H). Mass spec m/z 986.7 [M+H]⁺.

### Step 8

### Example 58: 1-(9-(2-(2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-5-fluoro-3-methyl-1H-indazole-6-carboxamido)-2-((R)-1-methylpyrrolidin-2-yl)-1H-pyrrolo[3,2-c]pyridine-1-carboxylate (Intermediate 254, 0.48 g, 0.48 mmol) in acetonitrile (10 mL) was added methanesulfonic acid (0.32 mL, 4.86 mmol) at room temperature and the reaction stirred at 50° C. for 2h. To the reaction mixture N,N′-dimethylethylenediamine (0.29 mL, 2.43 mmol) and triethylamine (1.36 mL, 9.72 mmol) were added. The reaction mixture was stirred at room temperature for 3 h. The reaction mixture was concentrated. The crude compound was quenched with water (50 mL) to get a solid precipitate which was filtered and dried in vacuo to afford the crude product. The crude material was purified by preparative HPLC (Method A) to afford 1-(9-(2-(2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide (Example 58, 0.08 g, 23%) as a white solid. ¹H NMR (400 MHz, DMSO-d₆) δ ppm 1.23-1.45 (m, 7H), 1.50-1.60 (m, 2H), 1.77-1.94 (m, 5H), 1.97-2.04 (m, 1H), 2.11-2.17 (m, 1H), 2.18 (s, 3H), 2.23-2.32 (m, 1H), 2.48 (s, 3H), 2.53-2.62 (m, 2H), 2.85-2.95 (m, 1H), 3.12-3.19 (m, 1H), 3.34-3.36 (m, 2H), 4.27-4.46 (m, 4H), 5.11-5.16 (m, 1H), 6.40 (s, 1H), 7.48-7.53 (m, 1H), 7.59-7.64 (m, 2H), 7.60-7.70 (m, 1H), 8.00 (d, J=5.50 Hz, 1H), 8.25 (s, 1H), 8.49 (s, 1H), 10.48 (s, 1H), 10.99 (br s, 1H), 11.40 (s, 1H). Mass spec m/z 756.9 [M+H]⁺.

### Example 59 was Synthesized Following Scheme 57

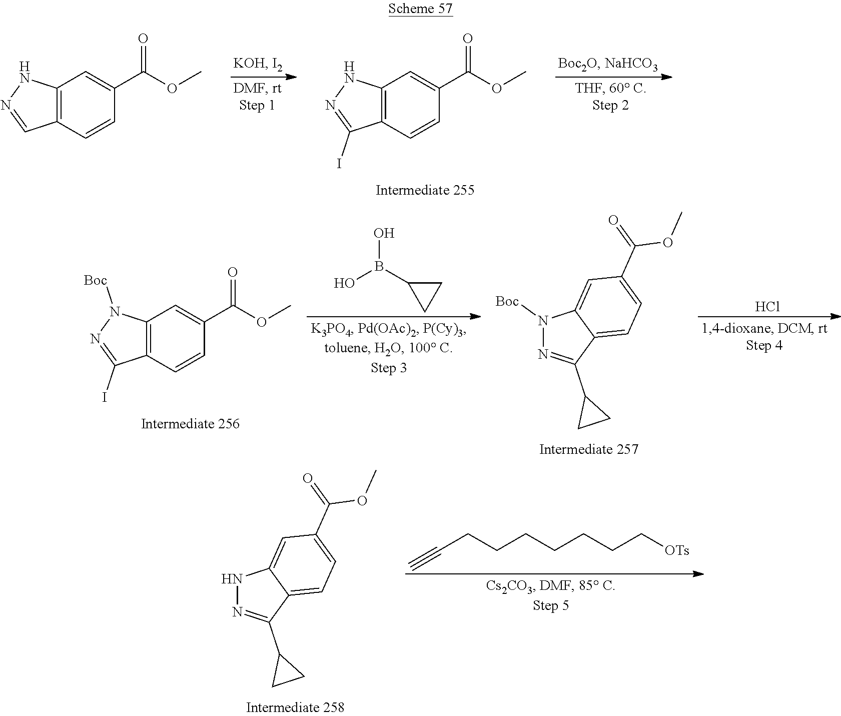

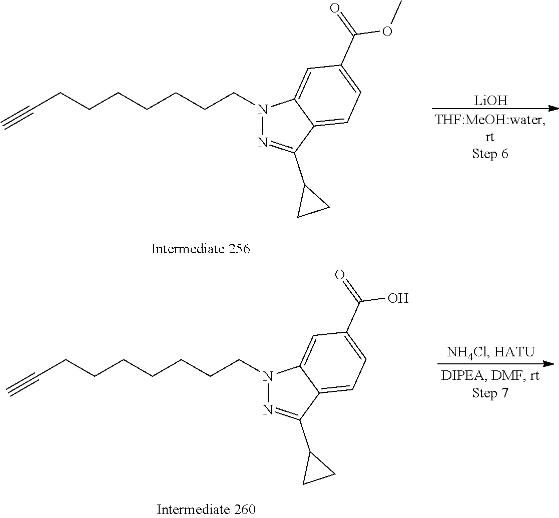

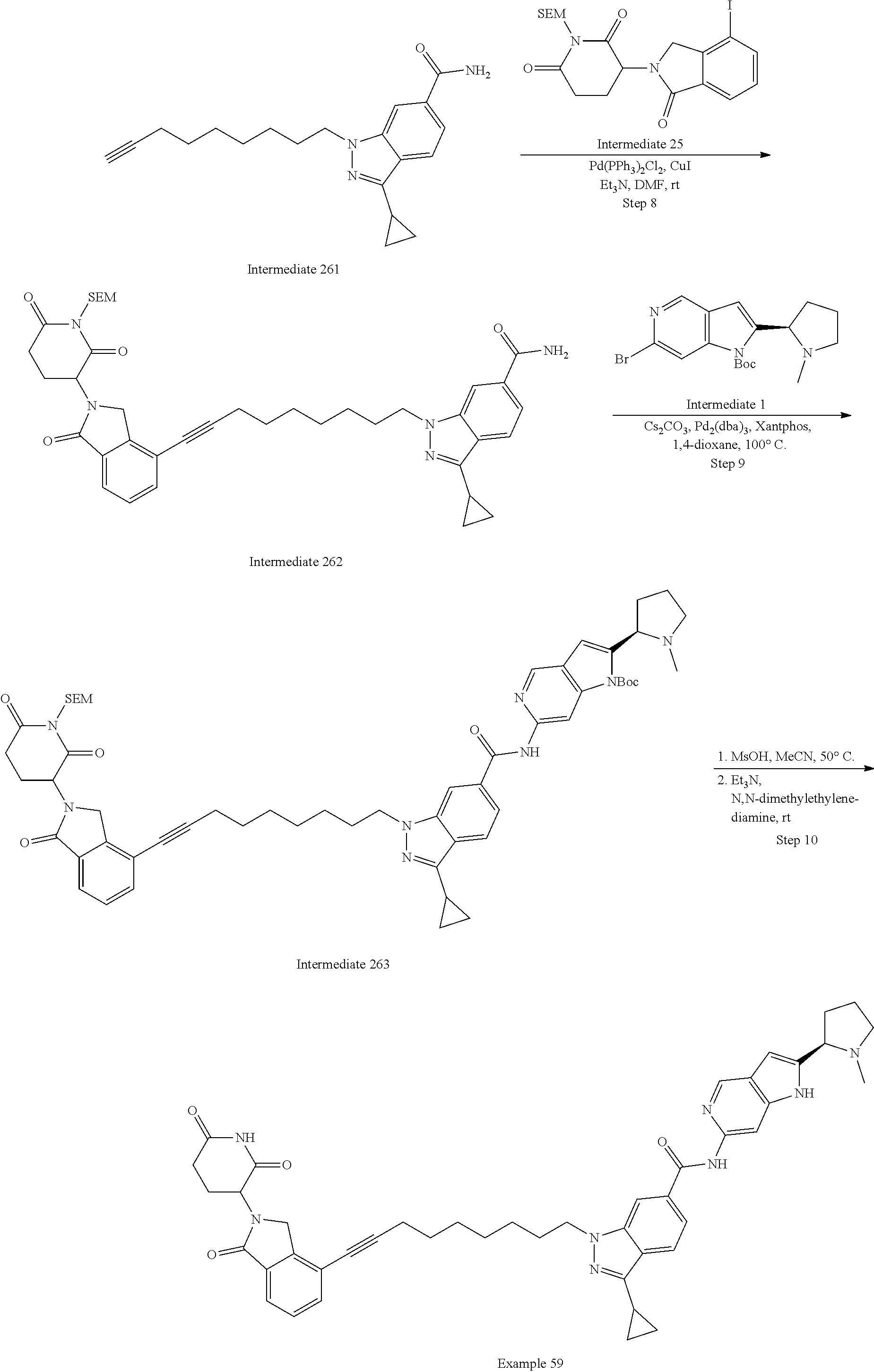 [SMILES: C#CCCCCCCCn1nc(C2CC2)c2ccc(C(N)=O)cc21 ; C[Si](C)(C)CCOCN1C(=O)CCC(N2Cc3c(I)cccc3C2=O)C1=O ; C[Si](C)(C)CCOCN1C(=O)CCC(N2Cc3c(C#CCCCCCCCn4nc(C5CC5)c5ccc(C(N)=O)cc54)cccc3C2=O)C1=O ; CN1CCC[C@@H]1c1cc2cnc(Br)cc2n1C(=O)OC(C)(C)C ; CN1CCC[C@@H]1c1cc2cnc(NC(=O)c3ccc4c(C5CC5)nn(CCCCCCCC#Cc5cccc6c5CN(C5CCC(=O)N(COCC[Si](C)(C)C)C5=O)C6=O)c4c3)cc2n1C(=O)OC(C)(C)C ; CN1CCC[C@@H]1c1cc2cnc(NC(=O)c3ccc4c(C5CC5)nn(CCCCCCCC#Cc5cccc6c5CN(C5CCC(=O)NC5=O)C6=O)c4c3)cc2[nH]1]

### Step 1

### Intermediate 255: methyl 3-iodo-1H-indazole-6-carboxylate

To a solution of methyl 1H-indazole-6-carboxylate (CAS No: 170487-40-8, 25.0 g, 141.91 mmol) in dimethylformamide (200 mL) was added potassium hydroxide (31.86 g, 567.63 mmol) and iodine (39.62 g, 156.10 mmol) and the reaction mixture was stirred at room temperature for 16 h. The reaction mixture was diluted with ethyl acetate (500 mL) and washed with ice-cold water (3×500 mL). The organic layer was separated, dried over anhydrous Na₂SO₄, filtered, and concentrated in vacuo to afford methyl 3-iodo-1H-indazole-6-carboxylate (Intermediate 255, 9.25 g, 22%) as an off-white solid, which was used for the next step without further purification. ¹H NMR (400 MHz, DMSO-d₆) δ ppm 3.90 (s, 3H), 7.56 (d, J=8.33 Hz, 1H), 7.75 (d, J=8.33 Hz, 1H), 8.16 (s, 1H), 13.86 (br s, 1H). Mass spec m/z 302.97 [M+H]⁺. Step 2 Intermediate 256: 1-(tert-butyl) 6-methyl 3-iodo-1H-indazole-1,6-dicarboxylate

To a solution of methyl 3-iodo-1H-indazole-6-carboxylate (Intermediate 255, 9.20 g, 30 mmol) in THF (90 mL) was added sodium bicarbonate (5.1 g, 61 mmol) followed by di-tert-butyl dicarbonate (11 g, 46 mmol) at room temperature. The reaction mixture was heated at 60° C. for 2 h. The reaction mixture was then concentrated in vacuo. The crude residue was quenched with water (100 mL) to get a solid precipitate which was filtered, washed with water (50 mL) and dried in vacuo to afford 1-(tert-butyl) 6-methyl 3-iodo-1H-indazole-1,6-dicarboxylate (Intermediate 256, 18.0 g) as an off-white solid which was used for the next step without further purification. ¹H NMR (400 MHz, DMSO-d₆) δ ppm 1.67 (s, 9H), 3.93 (s, 3H), 7.69 (d, J=8.33 Hz, 1H), 7.99 (d, J=8.77 Hz, 1H), 8.70 (s, 1H). Mass spec: m/z: 403.1 [M+H]⁺.

### Step 3

### Intermediate 257: 1-(tert-butyl) 6-methyl 3-cyclopropyl-1H-indazole-1,6-dicarboxylate

To a solution of 1-(tert-butyl) 6-methyl 3-iodo-1H-indazole-1,6-dicarboxylate (Intermediate 256, 15.0 g, 37.29 mmol) in toluene:water (1:1, 210 mL) was added potassium phosphate (32.3 g, 149.2 mmol). The reaction mixture was purged with argon gas for 15 min then cyclopropylboronic acid (CAS No: 411235-57-9, 6.54 g, 74.59 mmol), Pd(OAc)₂ (1.23 g, 7.46 mmol) and tricyclohexylphosphine (3.13 g, 11.19 mmol) were added. The reaction mixture was purged with argon gas for 10 min and heated at 100° C. for 16 h. The reaction mixture was then extracted with ethyl acetate (2×200 mL). The organic layer was separated, dried over anhydrous Na₂SO₄, filtered, and concentrated in vacuo. The crude material was purified by combi-flash column chromatography, by eluting with 12% ethyl acetate in heptane, to afford 1-(tert-butyl) 6-methyl 3-cyclopropyl-1H-indazole-1,6-dicarboxylate (Intermediate 257, 7.1 g, 60%) as yellow solid. ¹H NMR (400 MHz, DMSO-d₆) δ ppm 1.01-1.12 (m, 4H), 1.62 (s, 9H), 2.35-2.43 (m, 1H), 3.90 (s, 3H), 7.89 (d, J=8.33 Hz, 1H), 8.03 (d, J=8.33 Hz, 1H), 8.65 (s, 1H). Mass spec: m/z 317.3 [M+H]⁺.

### Step 4

### Intermediate 258: methyl 3-cyclopropyl-1H-indazole-6-carboxylate

To a solution of 1-(tert-butyl) 6-methyl 3-cyclopropyl-1H-indazole-1,6-dicarboxylate (Intermediate 257, 7.00 g, 22 mmol) in dichloromethane (30 mL) was added 4M HCl in 1,4-dioxane (70 mL) at room temperature and the reaction mixture was stirred for 16 h. The reaction mixture was quenched with saturated NaHCO₃ solution (100 mL) and extracted with ethyl acetate (2×100 mL). The organic layer was separated, dried over anhydrous Na₂SO₄, filtered, and concentrated in vacuo to afford methyl 3-cyclopropyl-1H-indazole-6-carboxylate (Intermediate 258, 4.5 g, 94%) as an orange solid, which was used for the next step without further purification. ¹H NMR (400 MHz, DMSO-d₆) δ ppm 0.92-1.04 (m, 4H), 2.27-2.36 (m, 1H), 3.89 (s, 3H), 7.64 (dd, J=8.50, 1.38 Hz, 1H), 7.89 (d, J=8.38 Hz, 1H), 8.06 (s, 1H), 12.93 (s, 1H). Mass spec: m/z 217.15 [M+H]⁺.

### Step 5

### Intermediate 259: methyl 3-cyclopropyl-1-(non-8-yn-1-yl)-1H-indazole-6-carboxylate

To a solution of methyl 3-cyclopropyl-1H-indazole-6-carboxylate (Intermediate 258, 3.5 g, 16 mmol) in dimethylformamide (35 mL) was added cesium carbonate (9.4 g, 49 mmol) followed by non-8-yn-1-yl 4-methylbenzenesulfonate (CAS No: 87462-64-4, 5.7 g, 19 mmol). The reaction mixture was heated at 85° C. for 4 h. The reaction mixture was quenched with ice cold water (50 mL) and extracted with ethyl acetate (2×50 mL). The organic layers were separated, dried over anhydrous Na₂SO₄, filtered, and concentrated in vacuo to obtain the crude compound as a mixture of regioisomers. The regioisomers were separated by combi-flash chromatography, by eluting with 6% ethyl acetate in heptane, to afford methyl 3-cyclopropyl-1-(non-8-yn-1-yl)-1H-indazole-6-carboxylate (Intermediate 259, 3.3 g, 60%) as yellow oil. ¹H NMR (400 MHz, DMSO-d₆) δ ppm 0.90-1.05 (m, 4H), 1.14-1.45 (m, 8H), 1.70-1.82 (m, 2H), 2.06-2.14 (m, 2H), 2.25-2.35 (m, 1H), 2.65-2.75 (m, 1H), 3.90 (s, 3H), 4.32-4.40 (m, 2H), 7.60-7.69 (m, 1H), 7.82-7.92 (m, 1H), 8.21 (s, 1H). Mass spec: m/z: 339.3 [M+H]⁺.

### Step 6

### Intermediate 260: 3-cyclopropyl-1-(non-8-yn-1-yl)-1H-indazole-6-carboxylic acid

To a solution of methyl 3-cyclopropyl-1-(non-8-yn-1-yl)-1H-indazole-6-carboxylate (Intermediate 59, 3.3 g, 9.8 mmol) in tetrahydrofuran:methanol:water (2:2:1, 50 mL) was added lithium hydroxide (0.94 g, 39 mmol) and the reaction mixture was stirred at room temperature for 16 h. The reaction mixture was then concentrated in vacuo, water (50 mL) was added and the mixture acidified to pH ~4 with 1N HCl and the aqueous phase extracted with ethyl acetate (2×50 mL). The organic layer was separated, dried over Na₂SO₄, and concentrated in vacuo to afford 3-cyclopropyl-1-(non-8-yn-1-yl)-1H-indazole-6-carboxylic acid (Intermediate 260, 3.0 g) as a yellow solid, which was used for the next step without further purification. ¹H NMR (401 MHz, DMSO-d₆) δ ppm 0.92-1.04 (m, 4H), 1.16-1.32 (m, 6H), 1.33-1.43 (m, 2H), 1.72-1.79 (m, 2H), 2.09-2.13 (m, 2H), 2.26-2.33 (m, 1H), 2.72 (t, J=2.63 Hz, 1H), 4.35 (t, J=6.91 Hz, 2H), 7.64 (d, J=8.19 Hz, 1H), 7.81 (d, J=8.31 Hz, 1H), 8.16 (s, 1H). Mass spec: m/z 325.25 [M+H]⁺.

### Step 7

### Intermediate 261: 3-cyclopropyl-1-(non-8-yn-1-yl)-1H-indazole-6-carboxamide

To a solution of 3-cyclopropyl-1-(non-8-yn-1-yl)-1H-indazole-6-carboxylic acid (Intermediate 260, 3.00 g, 9.25 mmol) in dimethylformamide (30 mL) was added HATU (5.55 g, 13.87 mmol) and DIPEA (5.75 mL, 32.37 mmol) at room temperature and stirred for 10 min. Ammonium chloride (1.62 mL, 46.24 mmol) was then added at 0° C. and the reaction mixture was stirred at room temperature for 16 h. The reaction mixture was then diluted with ice cold water (100 mL). The solid precipitate obtained was filtered and dried under vacuum to afford 3-cyclopropyl-1-(non-8-yn-1-yl)-1H-indazole-6-carboxamide (Intermediate 261, 2.5 g, 84%) as a white solid, which was used for next step without further purification. ¹H NMR (400 MHz, DMSO-d₆) δ ppm 0.91-1.04 (m, 4H), 1.17-1.43 (m, 7H), 1.72-1.82 (m, 2H), 2.07-2.15 (m, 2H), 2.23-2.35 (m, 2H), 2.66-2.73 (m, 1H), 4.30 (t, J=7.02 Hz, 2H), 7.41 (br s, 1H), 7.58 (d, J=8.33 Hz, 1H), 7.79 (d, J=8.33 Hz, 1H), 8.02 (br s, 1H), 8.10 (s, 1H). Mass spec: m/z 324.28 [M+H]⁺.

### Step 8

### Intermediate 262: 3-cyclopropyl-1-(9-(2-(2,6-dioxo-1-((2-(trimethylsilyl) ethoxy) methyl) piperidin-3-yl)-1-oxoisoindolin-4-yl) non-8-yn-1-yl)-1H-indazole-6-carboxamide

To a solution of 3-cyclopropyl-1-(non-8-yn-1-yl)-1H-indazole-6-carboxamide (Intermediate 261, 0.70 g, 2.16 mmol) in dimethylformamide (3 mL) was added 3-(4-iodo-1-oxo-isoindolin-2-yl)-1-(2-trimethylsilylethoxymethyl)piperidine-2,6-dione (Intermediate 25, 1.19 g, 2.38 mmol) followed by triethylamine (11.2 mL, 80.09 mmol). The reaction mixture was purged with argon for 15 min followed by the addition of copper(I) iodide (0.082 g, 0.43 mmol) and Pd(PPh₃)₂Cl₂ (0.15 g, 0.21 mmol), further purged with argon for 10 min and stirred at room temperature for 2 h. The reaction mixture was quenched with ice cold water (50 mL) and extracted with ethyl acetate (2×50 mL). The organic layer was separated, dried over anhydrous Na₂SO₄, and concentrated in vacuo. The crude material was purified by combi-flash column chromatography, by eluting with 3% MeOH in DCM, to afford 3-cyclopropyl-1-(9-(2-(2,6-dioxo-1-((2-(trimethylsilyl) ethoxy) methyl) piperidin-3-yl)-1-oxoisoindolin-4-yl) non-8-yn-1-yl)-1H-indazole-6-carboxamide (Intermediate 262, 0.80 g, 53%) as an off white solid. ¹H NMR (401 MHz, DMSO-d₆) δ ppm −0.04 (s, 9H), 0.78-0.87 (m, 2H), 0.89-1.02 (m, 4H), 1.19-1.27 (m, 2H), 1.29-1.41 (m, 4H), 1.48-1.56 (m, 2H), 1.72-1.83 (m, 2H), 1.99-2.08 (m, 1H), 2.23-2.30 (m, 1H), 2.39-2.47 (m, 4H), 2.75-2.80 (m, 1H), 3.00-3.12 (m, 1H), 3.47-3.55 (m, 2H), 4.26-4.33 (m, 2H), 4.43-4.47 (m, 1H), 4.99-5.09 (m, 2H), 5.25-5.30 (m, 1H), 7.44 (br s, 1H), 7.49-7.54 (m, 1H), 7.56-7.64 (m, 2H), 7.71 (d, J=6.97 Hz, 1H), 7.78 (d, J=8.44 Hz, 1H), 8.04 (br s, 1H), 8.10 (s, 1H). Mass spec: m/z: 696.47 [M+H]⁺.

### Step 9

### Intermediate 263: tert-butyl 6-(3-cyclopropyl-1-(9-(2-(2,6-dioxo-1-((2-(trimethylsilyl) ethoxy)methyl)piperidin-3-yl)-1-oxoisoindolin-4-yl)non-8-yn-1-yl)-1H-indazole-6-carboxamido)-2-((R)-1-methylpyrrolidin-2-yl)-1H-pyrrolo[3,2-c]pyridine-1-carboxylate

To a solution of 3-cyclopropyl-1-(9-(2-(2,6-dioxo-1-((2-(trimethylsilyl)ethoxy)methyl)piperidin-3-yl)-1-oxoisoindolin-4-yl non-8-yn-1-yl)-1H-indazole-6-carboxamide (Intermediate 262, 0.8 g, 1.15 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 1, 0.48 g, 1.26 mmol) followed by cesium carbonate (1.12 g, 3.45 mmol) at room temperature. The reaction mixture was purged with argon for 15 min followed by Pd₂(dba)₃ (0.16 g, 0.17 mmol) and Xantphos (0.19 g, 0.34 mmol). The reaction mixture was purged with argon for another 10 min and heated at 100° C. for 2 h. The reaction mixture was then concentrated in vacuo to obtain crude compound. The crude material was purified by combi-flash column chromatography, by eluting with 3% MeOH in DCM, to afford tert-butyl 6-(3-cyclopropyl-1-(9-(2-(2,6-dioxo-1-((2-(trimethylsilyl)ethoxy)methyl)piperidin-3-yl)-1-oxoisoindolin-4-yl)non-8-yn-1-yl)-1H-indazole-6-carboxamido)-2-((R)-1-methylpyrrolidin-2-yl)-1H-pyrrolo[3,2-c]pyridine-1-carboxylate (Intermediate 263, 0.6 g, 52%) as yellow solid. ¹H NMR (400 MHz, DMSO-d₆) δ ppm −0.05 (s, 9H), 0.73-0.88 (m, 2H), 0.89-1.06 (m, 4H), 1.16-1.44 (m, 6H), 1.48-1.57 (m, 2H), 1.58-1.66 (m, 2H), 1.70 (s, 9H), 1.73-1.88 (m, 3H), 1.98-2.08 (m, 1H), 2.25-2.33 (m, 1H), 2.36 (s, 3H), 2.40-2.46 (m, 3H), 2.54-2.56 (m, 2H), 2.74-2.80 (m, 2H), 2.99-3.19 (m, 2H), 3.48-3.54 (m, 2H), 3.86-3.97 (m, 1H), 4.22-4.26 (m, 1H), 4.33-4.47 (m, 2H), 4.96-5.10 (m, 2H), 5.24-5.28 (m, 1H), 6.76 (s, 1H), 7.50 (t, J=7.67 Hz, 1H), 7.60 (d, J=7.46 Hz, 1H), 7.65-7.74 (m, 2H), 7.84 (d, J=8.29 Hz, 1H), 8.41 (s, 1H), 8.60 (s, 1H), 8.97 (s, 1H), 10.73 (s, 1H). Mass spec: m/z: 995.61 [M+H]⁺.

### Step 10

### Example 59: 3-cyclopropyl-1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrolo [3,2-c]pyridin-6-yl)-1H-indazole-6-carboxamide

To a solution of tert-butyl 6-(3-cyclopropyl-1-(9-(2-(2,6-dioxo-1-((2-(trimethylsilyl)ethoxy)methyl)piperidin-3-yl)-1-oxoisoindolin-4-yl)non-8-yn-1-yl)-1H-indazole-6-carboxamido)-2-((R)-1-methylpyrrolidin-2-yl)-1H-pyrrolo[3,2-c]pyridine-1-carboxylate (Intermediate 263, 0.55 g, 0.55 mmol) in acetonitrile (6 mL) was added methanesulfonic acid (0.36 mL, 5.52 mmol) at room temperature and the reaction mixture was heated at 50° C. for 2h. N,N′-dimethylethylenediamine (0.33 mL, 2.76 mmol) was then added, followed by triethylamine (1.55 mL, 11.05 mmol) and the reaction stirred at room temperature for 4 h. The reaction mixture was then poured into ice cold water (50 mL) to get the solid precipitate which was filtered and dried under vacuum to obtain crude compound. The crude material was then purified by preparative HPLC (Method A) to afford 3-cyclopropyl-1-(9-(2-(2,6-dioxopiperidin-3-yl)-1-oxoisoindolin-4-yl)non-8-yn-1-yl)-N-(2-((R)-1-methylpyrrolidin-2-yl)-1H-pyrrolo[3,2-c]pyridin-6-yl)-1H-indazole-6-carboxamide (Example 59, 0.14 g, 34%) as an off white solid. ¹H NMR (400 MHz, DMSO-d₆) δ ppm 0.91-1.05 (m, 4H), 1.23-1.44 (m, 7H), 1.49-1.58 (m, 2H), 1.77-2.02 (m, 6H), 2.12-2.15 (m, 1H), 2.17 (s, 3H), 2.23-2.31 (m, 2H), 2.41-2.46 (m, 2H), 2.56-2.63 (m, 2H), 2.84-2.95 (m, 1H), 3.11-3.18 (m, 1H), 4.26-4.47 (m, 4H), 5.10-5.16 (m, 1H), 6.40 (s, 1H), 7.49 (t, J=7.57 Hz, 1H), 7.59 (d, J=6.88 Hz, 1H), 7.67-7.75 (m, 2H), 7.84 (d, J=8.50 Hz, 1H), 8.25 (s, 1H), 8.41 (s, 1H), 8.51 (s, 1H), 10.51 (s, 1H), 10.97 (br s, 1H), 11.38 (s, 1H). Mass spec: m/z: 765.2 [M+H]⁺.

### Example 60 was Synthesized Following Scheme 58

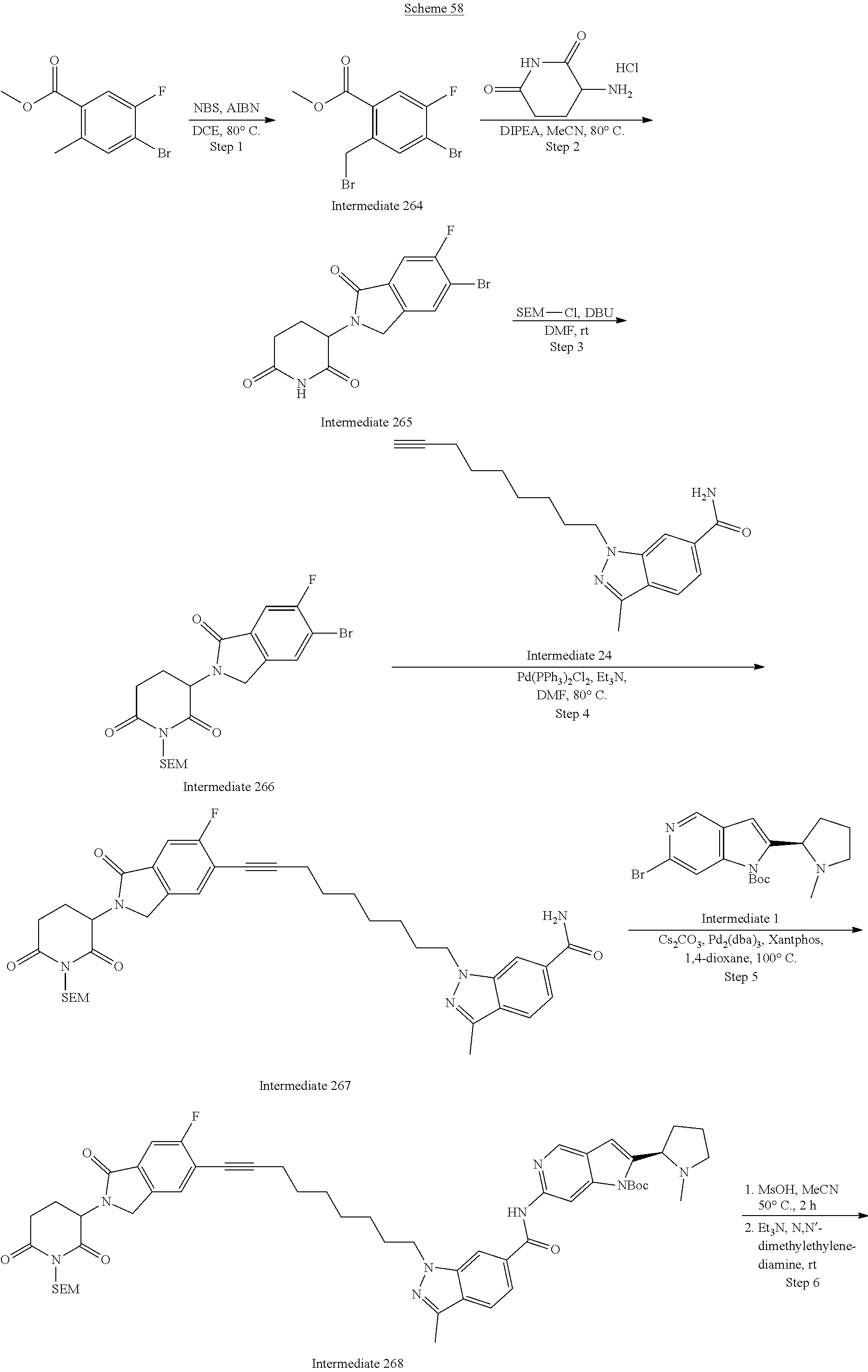

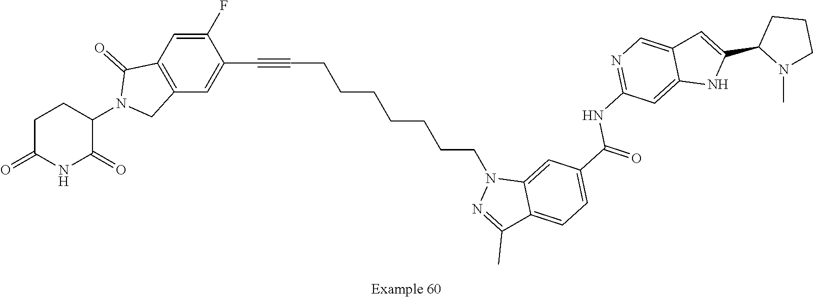 [SMILES: Cc1nn(CCCCCCCC#Cc2cc3c(cc2F)C(=O)N(C2CCC(=O)NC2=O)C3)c2cc(C(=O)Nc3cc4[nH]c([C@H]5CCCN5C)cc4cn3)ccc12]

### Step 1

### Intermediate 264: methyl 4-bromo-2-(bromomethyl)-5-fluorobenzoate

To a solution of methyl 4-bromo-5-fluoro-2-methylbenzoate (CAS No: 1352889-89-4, 5.00 g, 20 mmol) in dichloroethane (50 mL) was added N-bromosuccinimide (7.2 g, 40 mmol) followed by AIBN (0.66 g, 4.0 mmol). The reaction mixture was stirred at 80° C. for 12 h. The reaction mixture was poured into water (60 mL) and extracted with ethyl acetate (3×100 mL). The organic layer was separated, dried over Na₂SO₄, and concentrated in vacuo. The crude material was purified by combi-flash column chromatography, by eluting with 10% ethyl acetate in heptane, to afford methyl 4-bromo-2-(bromomethyl)-5-fluorobenzoate (Intermediate 264, 3.8 g, 58%) as an off white solid. ¹H NMR (400 MHz, DMSO-d) δ ppm 3.88 (s, 3H), 4.97 (s, 2H), 7.78 (d, J=9.21 Hz, 1H), 8.05 (d, J=7.02 Hz, 1H).

### Step 2

### Intermediate 265: 3-(5-bromo-6-fluoro-1-oxoisoindolin-2-yl)piperidine-2,6-dione

To a solution of methyl 4-bromo-2-(bromomethyl)-5-fluorobenzoate (Intermediate 264, 3.5 g, 11.0 mmol) in acetonitrile (40 mL) was added 3-aminopiperidine-2,6-dione hydrochloride (CAS No: 24666-56-6, 2.1 g, 13 mmol) followed by DIPEA (4.2 g, 32 mmol). The reaction mixture was stirred at 80° C. for 12 h. The reaction mixture was poured into cold water (50 mL), the resultant precipitate collected by filtration and dried in vacuo to afford 3-(5-bromo-6-fluoro-1-oxoisoindolin-2-yl) piperidine-2,6-dione (Intermediate 265, 3.00 g, 82%) as a blue solid, which was used for the next step without further purification. ¹H NMR (400 MHz, DMSO-d₆) δ ppm 1.97-2.06 (m, 1H), 2.33-2.43 (m, 1H), 2.56-2.62 (m, 1H), 2.84-2.98 (m, 1H), 4.29-4.50 (m, 2H), 5.09-5.14 (m, 1H), 7.70 (d, J=7.45 Hz, 1H), 8.04 (d, J=5.70 Hz, 1H), 11.00 (br s, 1H). Mass spec m/z 343.0 [M+H+2]⁺.

### Step 3

### Intermediate 266: 3-(5-bromo-6-fluoro-1-oxoisoindolin-2-yl)-1-((2-(trimethylsilyl)ethoxy)methyl) piperidine-2,6-dione

To a solution of 3-(5-bromo-6-fluoro-1-oxoisoindolin-2-yl) piperidine-2,6-dione (Intermediate 265, 2.7 g, 7.9 mmol) in dimethylformamide (30 mL) was added DBU (4.3 g, 28 mmol) at 0° C. followed by 2-(trimethylsilyl)ethoxymethyl chloride (5.1 mL, 28 mmol). The reaction mixture was stirred at room temperature for 16 h under nitrogen atmosphere. The reaction mixture was quenched with water (100 mL), diluted with ethyl acetate (200 mL) and filtered through a celite bed. The organic layer was separated, washed with water (150 mL), dried over anhydrous Na₂SO₄, filtered, and concentrated in vacuo. The crude material was purified by combi-flash chromatography, by eluting with 50% ethyl acetate in heptane, to afford 3-(5-bromo-6-fluoro-1-oxoisoindolin-2-yl)-1-((2-(trimethylsilyl)ethoxy)methyl) piperidine-2,6-dione (Intermediate 266, 1.7 g, 46%) as brown solid. ¹H NMR (400 MHz, DMSO-d₆) δ ppm 0.02 (s, 9H), 1.81-1.86 (m, 2H), 2.04-2.10 (m, 1H), 2.36-2.42 (m, 1H), 2.76-2.81 (m, 1H), 3.01-3.08 (m, 1H), 3.49-3.55 (m, 2H), 4.29-4.33 (m, 1H), 4.46-4.50 (m, 1H), 5.01-5.08 (m, 2H), 5.20-5.24 (m, 1H), 7.71 (d, J=7.45 Hz, 1H), 8.05 (d, J=5.70 Hz, 1H). Mass spec m/z 469.0 [M−H]—.

### Step 4

### Intermediate 267: 1-(9-(2-(2,6-dioxo-1-((2-(trimethylsilyl)ethoxy)methyl)piperidin-3-yl)-6-fluoro-1-oxoisoindolin-5-yl) non-8-yn-1-yl)-3-methyl-1H-indazole-6-carboxamide

To a solution of 3-(5-bromo-6-fluoro-1-oxoisoindolin-2-yl)-1-((2-(trimethylsilyl)ethoxy)methyl)piperidine-2,6-dione (Intermediate 266, 0.50 g, 1.06 mmol) in dimethylformamide (5 mL) was added 3-methyl-1-(non-8-yn-1-yl)-1H-indazole-6-carboxamide (Intermediate 24, 1.57 g, 5.30 mmol) followed by triethylamine (0.44 mL, 3.18 mmol) at room temperature. The reaction mixture was purged with argon for 15 min followed by the addition of PdCl₂(PPh₃)₂ (0.076 g, 0.10 mmol). The reaction mixture was further purged with argon for 10 min and stirred at 80° C. for 16 h. The reaction mixture was poured into water (30 mL) and extracted with ethyl acetate (3×50 mL). The organic layer was separated, dried over Na₂SO₄, and concentrated in vacuo. The crude material was purified by combi-flash chromatography, by eluting with 100% ethyl acetate in heptane, to afford 1-(9-(2-(2,6-dioxo-1-((2-(trimethylsilyl) ethoxy) methyl) piperidin-3-yl)-6-fluoro-1-oxoisoindolin-5-yl) non-8-yn-1-yl)-3-methyl-1H-indazole-6-carboxamide (Intermediate 267, 0.50 g, 69%) as an off white solid. ¹H NMR (400 MHz, DMSO-d₆) δ ppm 0.00 (s, 9H), 0.83-0.90 (m, 2H), 1.24-1.47 (m, 6H), 1.52-1.60 (m, 2H), 1.82-1.90 (m, 2H), 2.04-2.12 (m, 1H), 2.34-2.44 (m, 1H), 2.45-2.48 (m, 2H), 2.51 (s, 3H), 2.77-2.86 (m, 1H), 3.03-3.14 (m, 1H), 3.50-3.58 (m, 2H), 4.25-4.40 (m, 3H), 4.42-4.50 (m, 1H), 5.03-5.11 (m, 2H), 5.24-5.28 (m, 1H), 7.45 (br s, 1H), 7.58-7.64 (m, 2H), 7.72-7.77 (m, 2H), 8.07 (br s, 1H), 8.15 (s, 1H). Mass spec m/z 688.56 [M+H]⁺.

### Step 5

### Intermediate 268: tert-butyl 6-(1-(9-(2-(2,6-dioxo-1-((2-(trimethylsilyl)ethoxy)methyl)piperidin-3-yl)-6-fluoro-1-oxoisoindolin-5-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(9-(2-(2,6-dioxo-1-((2-(trimethylsilyl) ethoxy) methyl) piperidin-3-yl)-6-fluoro-1-oxoisoindolin-5-yl) non-8-yn-1-yl)-3-methyl-1H-indazole-6-carboxamide (Intermediate 267, 0.50 g, 0.73 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 1, 0.35 g, 0.92 mmol) followed by cesium carbonate (0.60 g, 1.84 mmol) at room temperature. The reaction mixture was purged with argon for 15 min followed by addition of Pd₂(dba)₃ (0.13 g, 0.14 mmol) and Xantphos (0.16 g, 0.27 mmol). The reaction mixture was purged with argon for another 10 min and stirred at 100° C. for 2 h. The reaction mixture was concentrated in vacuo to obtain the crude compound. The crude material was purified by combi-flash column chromatography, by eluting with 80% ethyl acetate in heptane, to afford tert-butyl 6-(1-(9-(2-(2,6-dioxo-1-((2-(trimethylsilyl)ethoxy)methyl)piperidin-3-yl)-6-fluoro-1-oxoisoindolin-5-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 268, 0.30 g, 33%) as a yellow solid. ¹H NMR (400 MHz, DMSO-d₆) δ ppm −0.03 (s, 9H), 0.79-0.88 (m, 2H), 1.21-1.47 (m, 6H), 1.49-1.66 (m, 4H), 1.70 (s, 9H), 1.74-1.78 (m, 2H), 1.83-1.93 (m, 2H), 2.01-2.09 (m, 1H), 2.28-2.33 (m, 3H), 2.36 (s, 3H), 2.52 (s, 3H), 2.64-2.68 (m, 1H), 2.74-2.82 (m, 1H), 2.99-3.17 (m, 2H), 3.48-3.56 (m, 2H), 3.90-3.96 (m, 1H), 4.22-4.27 (m, 1H), 4.35-4.46 (m, 3H), 4.98-5.09 (m, 2H), 5.20-5.26 (m, 1H), 6.76 (s, 1H), 7.55 (d, J=8.40 Hz, 1H), 7.66-7.74 (m, 2H), 7.78 (d, J=8.40 Hz, 1H), 8.43 (s, 1H), 8.61 (s, 1H), 8.97 (s, 1H), 10.75 (br s, 1H). Mass spec m/z 986.6 [M+H]⁺.

### Step 6

### Example 60: 1-(9-(2-(2,6-dioxopiperidin-3-yl)-6-fluoro-1-oxoisoindolin-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(9-(2-(2,6-dioxo-1-((2-(trimethylsilyl)ethoxy)methyl)piperidin-3-yl)-6-fluoro-1-oxoisoindolin-5-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 268, 0.30 g, 0.30 mmol) in acetonitrile (10 mL) was added methanesulfonic acid (0.20 mL, 3.04 mmol) at room temperature and the reaction mixture was stirred at 50° C. for 2 h. After cooling to room temperature, N,N′-dimethylethylenediamine (0.18 mL, 1.52 mmol) and triethylamine (0.85 mL, 6.07 mmol) were added and the reaction stirred at room temperature for 3 h. The reaction mixture was poured into ice cold water (20 mL) and the resultant precipitate collected by filtration and dried in vacuo. The crude compound was purified by preparative HPLC (Method A) to afford 1-(9-(2-(2,6-dioxopiperidin-3-yl)-6-fluoro-1-oxoisoindolin-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 60, 0.13 g, 56%) as an off white solid. ¹H NMR (400 MHz, DMSO-d₆) δ ppm 1.31-1.40 (m, 6H), 1.52-1.56 (m, 2H), 1.84-2.01 (m, 6H), 2.14-2.16 (m, 1H), 2.17 (s, 3H), 2.25-2.28 (m, 1H), 2.31-2.33 (m, 1H), 2.46 (s, 3H), 2.56-2.63 (m, 2H), 2.85-2.94 (m, 1H), 3.09-3.16 (m, 1H), 3.31-3.35 (m, 2H), 4.31-4.43 (m, 4H), 5.08-5.12 (m, 1H), 6.40 (s, 1H), 7.55 (d, J=8.00 Hz, 1H), 7.67-7.79 (m, 3H), 8.25 (s, 1H), 8.44 (s, 1H), 8.52 (s, 1H), 10.52 (s, 1H), 10.99 (s, 1H), 11.38 (s, 1H). Mass spec m/z 757.1 [M+H]⁺.

### Example 61 was Synthesized Following Scheme 59

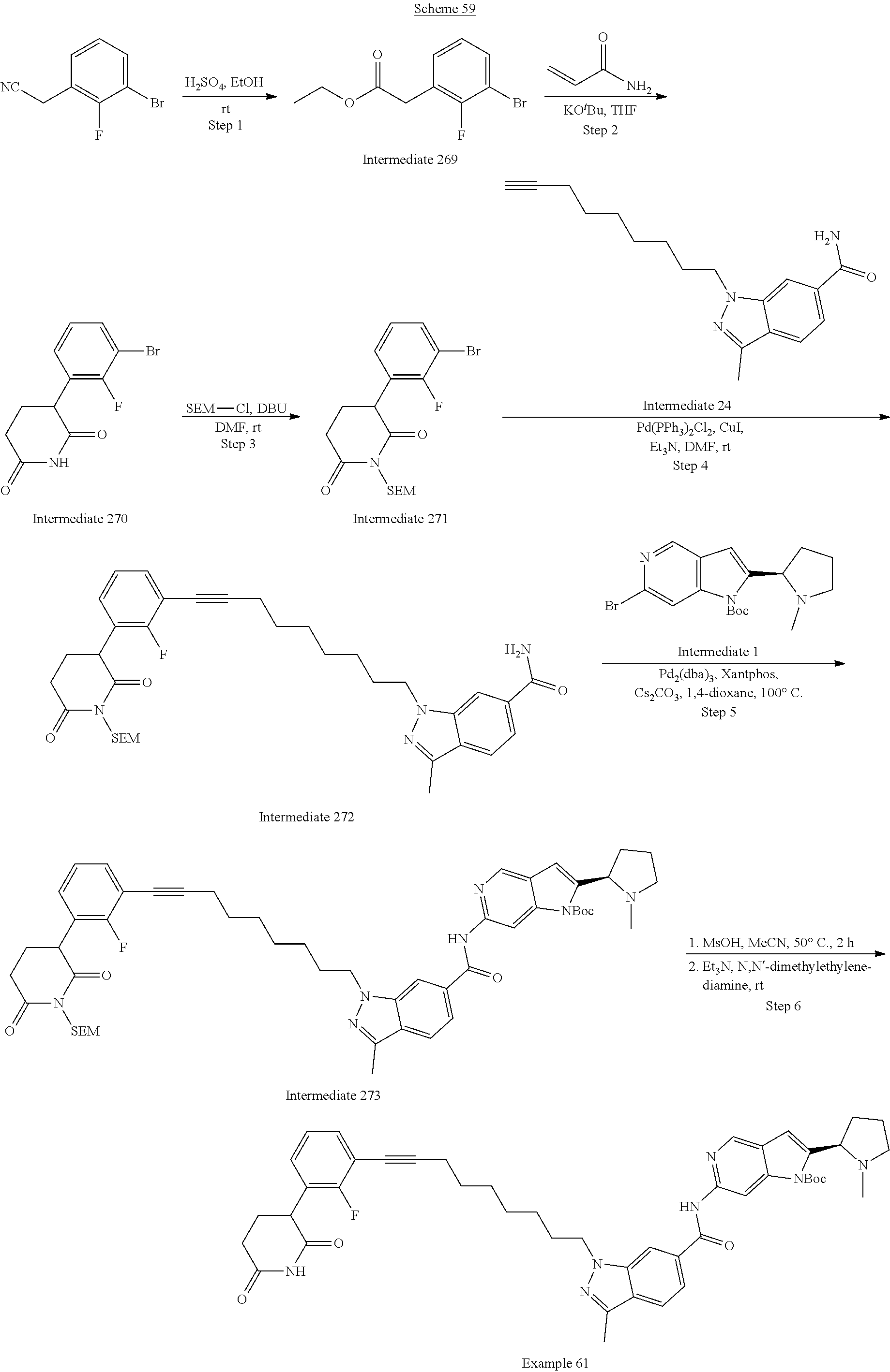

### Step 1

### Intermediate 269: ethyl 2-(3-bromo-2-fluorophenyl)acetate

To a solution of 2-(3-bromo-2-fluorophenyl) acetonitrile (CAS No: 874285-03-7, 15.0 g, 70.08 mmol) in ethanol (150 mL) was added sulfuric acid (25 mL) and the reaction mixture was stirred at room temperature for 3 h. The reaction mixture was concentrated in vacuo. The obtained residue was poured into water (1500 mL), adjusted to pH~4 by addition of 1M aqueous HCl and extracted with dichloromethane (3×500 mL). The organic layer was separated, dried over Na₂SO₄, filtered and concentrated in vacuo to afford ethyl 2-(3-bromo-2-fluorophenyl) acetate (Intermediate 269, 14.0 g, 73%) as colourless oil, which was used for the next step without further purification. ¹H NMR (400 MHz, DMSO-d₆) δ ppm 1.19 (t, J=7.02 Hz, 3H), 3.79 (s, 2H), 4.08-4.14 (m, 2H), 7.10-7.14 (m, 1H), 7.35-7.38 (m, 1H), 7.60-7.63 (m, 1H). Mass spec m/z 260.1 [M+H]⁺.

### Step 2

### Intermediate 270: 3-(3-bromo-2-fluorophenyl) piperidine-2,6-dione

To a solution of ethyl 2-(3-bromo-2-fluorophenyl) acetate (Intermediate 269, 5.00 g, 19 mmol) in tetrahydrofuran (50 mL) was added acrylamide (CAS No: 79-06-1, 1.00 g, 19 mmol) followed by KO^{t}Bu (2.1 mL, 23 mmol) at 0° C. The reaction mixture was stirred at room temperature for 12 h. The reaction mixture was diluted with water (100 mL) and extracted with ethyl acetate (3×100 mL). The organic layer was separated, dried over Na₂SO₄, filtered and concentrated in vacuo. The crude material was purified by combi-flash chromatography, by eluting with 100% ethyl acetate in heptane, to afford 3-(3-bromo-2-fluorophenyl) piperidine-2,6-dione (Intermediate 270, 2.00 g, 37%) as an off white solid. ¹H NMR (401 MHz, DMSO-d₆) δ ppm 1.98-2.08 (m, 1H), 2.19-2.31 (m, 1H), 2.55-2.60 (m, 1H), 2.68-2.82 (m, 1H), 4.12-4.17 (m, 1H), 7.14-7.18 (m, 1H), 7.33-7.40 (m, 1H), 7.62-7.69 (m, 1H), 10.96 (br s, 1H). Mass spec m/z 287.79 [M+H+2]⁺.

### Step 3

### Intermediate 271: 3-(3-bromo-2-fluorophenyl)-1-((2-(trimethylsilyl) ethoxy) methyl) piperidine-2,6-dione

To a cooled (0° C.) solution of 3-(3-bromo-2-fluorophenyl) piperidine-2,6-dione (Intermediate 270, 2.00 g, 7.0 mmol) in dimethylformamide (20 mL) was added DBU (3.7 g, 24 mmol) followed by 2-(trimethylsilyl)ethoxymethyl chloride (4.1 g, 24 mmol). The reaction mixture was stirred at room temperature for 16 h. The reaction mixture was diluted with water (100 mL) and extracted with ethyl acetate (3×100 mL). The organic layer was separated, dried over anhydrous Na₂SO₄, filtered and concentrated in vacuo. The crude material was purified by combi-flash chromatography, by eluting with 30% ethyl acetate in heptane, to afford 3-(3-bromo-2-fluorophenyl)-1-((2-(trimethylsilyl) ethoxy) methyl) piperidine-2,6-dione (Intermediate 271, 2.6 g, 89%) as an off white solid. ¹H NMR (400 MHz, DMSO-d₆) δ ppm −0.02 (s, 9H), 0.79-0.89 (m, 2H), 1.99-2.08 (m, 1H), 2.18-2.30 (m, 1H), 2.70-2.79 (m, 1H), 2.85-2.97 (m, 1H), 3.48-3.56 (m, 2H), 4.26-4.30 (m, 1H), 5.09 (s, 2H), 7.14-7.18 (m, 1H), 7.33-7.37 (m, 1H), 7.63-7.67 (m, 1H).

### Step 4

### Intermediate 272: 1-(9-(3-(2,6-dioxo-1-((2-(trimethylsilyl)ethoxy)methyl) piperidin-3-yl)-2-fluorophenyl) non-8-yn-1-yl)-3-methyl-1H-indazole-6-carboxamide

To a solution of 3-(3-bromo-2-fluorophenyl)-1-((2-(trimethylsilyl) ethoxy) methyl) piperidine-2,6-dione (Intermediate 271, 0.70 g, 2 mmol) in dimethylformamide (5.0 mL) was added 3-methyl-1-(non-8-yn-1-yl)-1H-indazole-6-carboxamide (Intermediate 24, 0.50 g, 2.00 mmol) followed by triethylamine (0.7 mL, 5 mmol) at room temperature. The reaction mixture was purged with argon for 15 min then copper (I) iodide (0.03 g, 0.2 mmol) and PdCl₂(PPh₃)₂ (0.1 g, 0.2 mmol) were added. The reaction mixture was purged with argon for 10 min and stirred at room temperature for 3 h. The reaction mixture was diluted with water (100 mL) and extracted with ethyl acetate (3×100 mL). The organic layer was separated, dried over Na₂SO₄, and concentrated in vacuo. The crude material was purified by combi-flash column chromatography, by eluting with 80% ethyl acetate in heptane, to afford 1-(9-(3-(2,6-dioxo-1-((2-(trimethylsilyl) ethoxy) methyl) piperidin-3-yl)-2-fluorophenyl) non-8-yn-1-yl)-3-methyl-1H-indazole-6-carboxamide (Intermediate 272, 0.70 g, 70%) as a yellow solid. ¹H NMR (400 MHz, DMSO-d₆) δ ppm −0.03 (s, 9H), 0.80-0.87 (m, 2H), 1.19-1.43 (m, 6H), 1.45-1.56 (m, 2H), 1.77-1.88 (m, 2H), 1.96-2.06 (m, 1H), 2.15-2.25 (m, 1H), 2.41-2.45 (m, 2H), 2.49 (s, 3H), 2.70-2.77 (m, 1H), 2.85-2.92 (m, 1H), 3.48-3.57 (m, 2H), 4.18-4.22 (m, 1H), 4.31-4.35 (m, 2H), 5.09 (s, 2H), 7.12-7.18 (m, 1H), 7.26-7.31 (m, 1H), 7.34-7.44 (m, 2H), 7.59 (d, J=8.40 Hz, 1H), 7.72 (d, J=8.40 Hz, 1H), 8.02 (br s, 1H), 8.12 (s, 1H). Mass spec m/z 633.40 [M+H]⁺.

### Step 5

### Intermediate 273: tert-butyl 6-(1-(9-(3-(2,6-dioxo-1-((2-(trimethylsilyl)ethoxy)methyl)piperidin-3-yl)-2-fluorophenyl)non-8-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(9-(3-(2,6-dioxo-1-((2-(trimethylsilyl) ethoxy) methyl) piperidin-3-yl)-2-fluorophenyl) non-8-yn-1-yl)-3-methyl-1H-indazole-6-carboxamide (Intermediate 272, 0.40 g, 0.63 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 1, 0.19 g, 0.50 mmol) followed by cesium carbonate (0.62 g, 1.89 mmol) at room temperature. The reaction mixture was purged with argon for 15 min then Pd₂(dba)₃ (0.059 g, 0.063 mmol) and Xantphos (0.11 g, 0.18 mmol) were added. The reaction mixture was purged with argon for 10 min and stirred at 100° C. for 2h. The reaction mixture was concentrated in vacuo to obtain crude compound. The crude material was purified by combi-flash column chromatography, by eluting with 90% ethyl acetate in heptane, to afford tert-butyl 6-(1-(9-(3-(2,6-dioxo-1-((2-(trimethylsilyl)ethoxy)methyl)piperidin-3-yl)-2-fluorophenyl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 273, 0.56 g, 95%) as a white solid. ¹H NMR (400 MHz, DMSO-d₆) δ ppm −0.05 (s, 9H), 0.78-0.87 (m, 2H), 1.23-1.44 (m, 6H), 1.51-1.66 (m, 2H), 1.68-1.70 (m, 2H), 1.71 (s, 9H), 1.74-1.78 (m, 2H), 1.82-1.92 (m, 2H), 2.01-2.09 (m, 1H), 2.28-2.33 (m, 2H), 2.36 (s, 3H), 2.40-2.44 (m, 2H), 2.50 (s, 3H), 2.65-2.77 (m, 1H), 2.82-2.95 (m, 1H), 3.12-3.16 (m, 1H), 3.45-3.57 (m, 2H), 3.90-4.06 (m, 1H), 4.16-4.21 (m, 1H), 4.40 (t, J=6.58 Hz, 2H), 5.08 (s, 2H), 6.76 (s, 1H), 7.09-7.16 (m, 1H), 7.27 (t, J=6.80 Hz, 1H), 7.36 (t, J=6.80 Hz, 1H), 7.70-7.74 (m, 1H), 7.76-7.84 (m, 1H), 8.43 (s, 1H), 8.61 (s, 1H), 8.98 (s, 1H), 10.76 (s, 1H). Mass spec m/z 932.73 [M+H]⁺.

### Step 6

### Example 61: 1-(9-(3-(2,6-dioxopiperidin-3-yl)-2-fluorophenyl) non-8-yn-1-yl)-3-methyl-N-(2-((R)-1-methylpyrrolidin-2-yl)-1H-pyrrolo [3,2-c]pyridin-6-yl)-1H-indazole-6-carboxamide

To a solution of tert-butyl 6-(1-(9-(3-(2,6-dioxo-1-((2-(trimethylsilyl)ethoxy)methyl)piperidin-3-yl)-2-fluorophenyl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 273, 0.40 g, 0.43 mmol) in acetonitrile (10 mL) was added methanesulfonic acid (0.42 mL, 6.43 mmol) at room temperature and the reaction mixture was stirred at 50° C. for 2h. To the reaction mixture was added N,N′-dimethylethylenediamine (0.31 mL, 2.57 mmol) followed by triethylamine (0.87 g, 8.58 mmol) and the reaction stirred at room temperature for 3 h. The reaction mixture was poured into ice cold water (100 mL), the resultant precipitate collected by filtration and dried in vacuo to afford the crude compound. The crude material was purified by preparative HPLC (Method A) to afford 1-(9-(3-(2,6-dioxopiperidin-3-yl)-2-fluorophenyl) non-8-yn-1-yl)-3-methyl-N-(2-((R)-1-methylpyrrolidin-2-yl)-1H-pyrrolo [3,2-c]pyridin-6-yl)-1H-indazole-6-carboxamide (Example 61, 0.11 g, 37%) as an off white solid. ¹H NMR (400 MHz, DMSO-d₆) δ ppm 1.31-1.40 (m, 6H), 1.52-1.56 (m, 2H), 1.84-2.01 (m, 6H), 2.17 (s, 3H), 2.22-2.43 (m, 3H), 2.45-2.48 (m, 2H), 2.51 (s, 3H), 2.56-2.60 (m, 1H), 2.66-2.74 (m, 1H), 3.12-3.16 (m, 1H), 3.32-3.34 (m, 1H), 4.03-4.07 (m, 1H), 4.38-4.42 (m, 2H), 6.40 (s, 1H), 7.09-7.14 (m, 1H), 7.25-7.28 (m, 1H), 7.32-7.36 (m, 1H), 7.72-7.79 (m, 2H), 8.25 (s, 1H), 8.44 (s, 1H), 8.52 (s, 1H), 10.52 (s, 1H), 10.89 (s, 1H), 11.38 (s, 1H). Mass spec m/z 702.1 [M+H]⁺.

### Example 62 was Synthesized Following Scheme 60

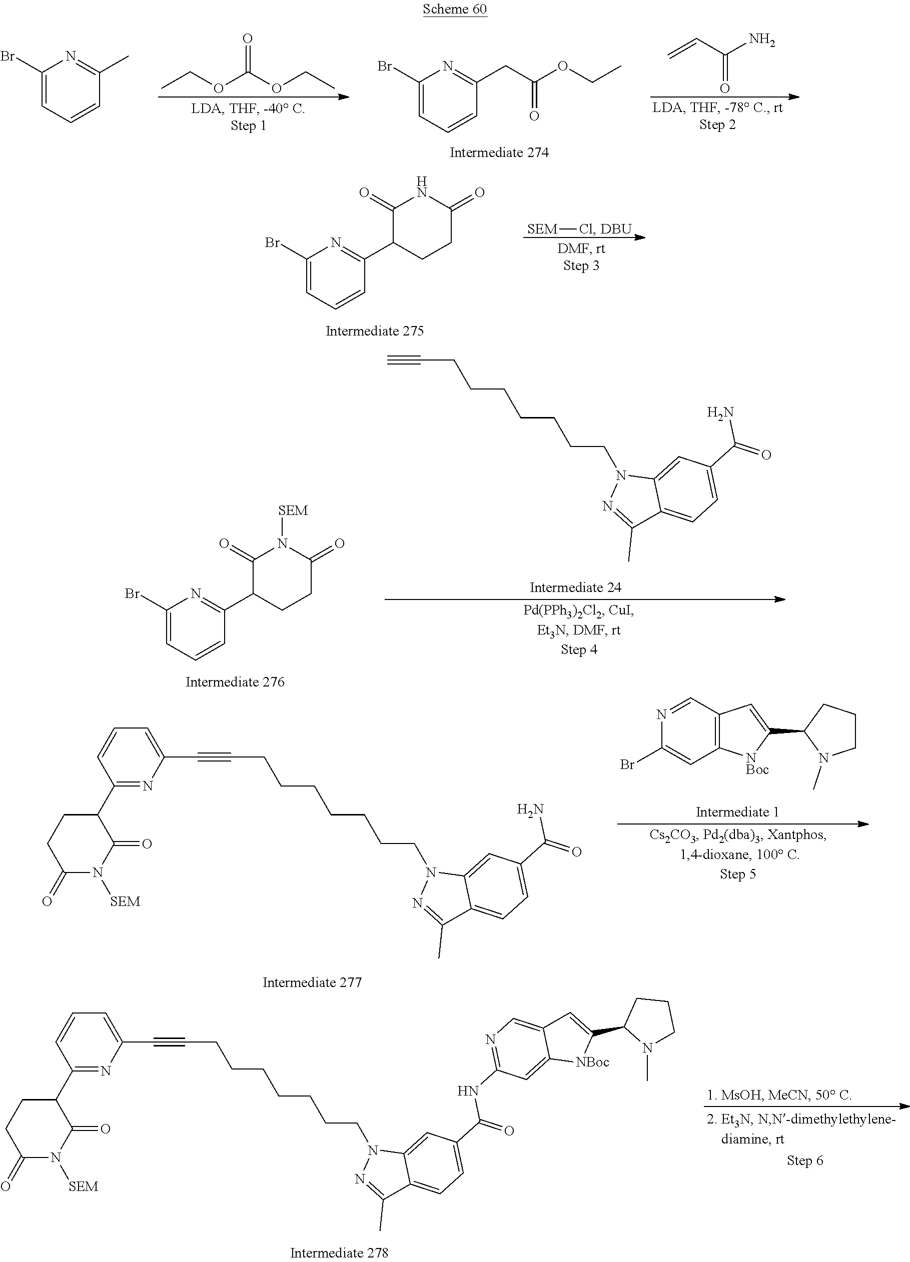

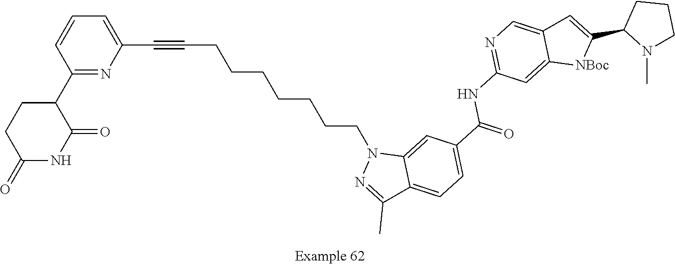 [SMILES: Cc1nn(CCCCCCCC#Cc2cccc(C3CCC(=O)NC3=O)n2)c2cc(C(=O)Nc3cc4c(cn3)cc([C@H]3CCCN3C)n4C(=O)OC(C)(C)C)ccc12]

### Step 1

### Intermediate 274: ethyl 2-(6-bromopyridin-2-yl) acetate

To a cooled (−40° C.) solution of 2-bromo-6-methylpyridine (CAS No: 5315-25-3, 10.0 g, 58.13 mmol) in anhydrous tetrahydrofuran (100 mL) was added lithium diisopropylamide (2M in THF solution, 116 mL, 116.27 mmol) and the reaction mixture was stirred for 10 min at −40pC. Then diethyl carbonate (CAS No: 105-58-8, 13.73 g, 116.27 mmol) w was added and the mixture was stirred at −40aC for 2 h. The reaction mixture was quenched with saturated NH₄Cl solution (200 mL) and extracted with ethyl acetate (3×200 mL). The combined organic layers were separated, dried over anhydrous Na₂SO₄, filtered, and concentrated in vacuo. The crude material was purified by combi-flash chromatography, by eluting with 20% ethyl acetate in heptane, to afford ethyl 2-(6-bromopyridin-2-yl) acetate (Intermediate 274, 5.00 g, 35%) as a colourless liquid. ¹H NMR (401 MHz, DMSO-d₆) δ ppm 1.18 (t, J=7.20 Hz, 3H), 3.85 (s, 2H), 4.10 (q, J=7.20 Hz, 2H), 7.42 (d, J=7.60 Hz, 1H), 7.55 (d, J=8.00 Hz, 1H), 7.74 (t, J=7.60 Hz, 1H). Mass spec m/z 244.09 [M+H]⁺.

### Step 2

### Intermediate 275: 3-(6-bromopyridin-2-yl)piperidine-2,6-dione

To a cooled (−78° C.) solution of ethyl-2-(6-bromopyridin-2-yl)acetate (Intermediate 274, 3.30 g, 14 mmol) in tetrahydrofuran (20 mL) was added acrylamide (CAS No: 79-06-1, 1.00 g, 14 mmol) and the reaction mixture was stirred for 10 min at −78° C. Then lithium diisopropylamide (2M in THF solution, 16 mL, 16 mmol) was added and the mixture was stirred at −78° C. for 1 h, then allowed to warm to room temperature and stirred for a further 3 h. The reaction mixture was quenched with saturated NH₄Cl solution (20 mL) and extracted with ethyl acetate (2×25 mL). The combined organic layers were separated, dried over anhydrous Na₂SO₄, filtered, and concentrated in vacuo. The crude material was purified by combi-flash chromatography, by eluting with 50% ethyl acetate in heptane, to afford 3-(6-bromopyridin-2-yl) piperidine-2,6-dione (Intermediate 275, 0.90 g, 25%) as a white solid. ¹H NMR (400 MHz, DMSO-d₆) δ ppm 2.06-2.14 (m, 1H), 2.19-2.31 (m, 1H), 2.54-2.58 (m, 1H), 2.61-2.68 (m, 1H), 4.02-4.08 (m, 1H), 7.42 (d, J=7.60 Hz, 1H), 7.57 (d, J=8.0 Hz, 1H), 7.75 (t, J=7.60 Hz, 1H), 10.91 (br s, 1H). Mass spec m/z 269.2 [M+H]⁺.

### Step 3

### Intermediate 276: 3-(6-bromopyridin-2-yl)-1-((2-(trimethylsilyl) ethoxy) methyl) piperidine-2,6-dione

To a cooled (0° C.) solution of 3-(6-bromopyridin-2-yl) piperidine-2,6-dione (Intermediate 275, 0.70 g, 2.60 mmol) in dimethylformamide (10 mL) was added DBU (0.60 mL, 3.90 mmol), followed by 2-(trimethylsilyl)ethoxymethyl chloride (0.72 mL, 3.90 mmol). The reaction mixture was stirred at room temperature for 16 h, then quenched with water (50 mL) and extracted with ethyl acetate (100 mL). The organic layer was separated, dried over anhydrous Na₂SO₄, filtered, and concentrated in vacuo. The crude material was purified by combi-flash chromatography, by eluting with 50% ethyl acetate in heptane, to afford 3-(6-bromopyridin-2-yl)-1-((2-(trimethylsilyl)ethoxy)methyl)piperidine-2,6-dione (Intermediate 276, 0.72 g, 45%) as an off-white solid. ¹H NMR (400 MHz, DMSO-d₆) δ ppm −0.03 (s, 9H), 0.79-0.88 (m, 2H), 2.06-2.17 (m, 1H), 2.20-2.32 (m, 1H), 2.57-2.58 (m, 1H), 2.61-2.68 (m, 1H), 3.47-3.58 (m, 2H), 4.21-4.24 (m, 1H), 5.08 (s, 2H), 7.43 (d, J=7.60 Hz, 1H), 7.58 (d, J=8.0 Hz, 1H), 7.76 (t, J=8.0 Hz, 1H). Mass spec m/z 399.0 [M+H]⁺.

### Step 4

### Intermediate 277: 1-(9-(6-(2,6-dioxo-1-((2-(trimethylsilyl) ethoxy) methyl) piperidin-3-yl) pyridin-2-yl) non-8-yn-1-yl)-3-methyl-1H-indazole-6-carboxamide

To a solution of 3-methyl-1-(non-8-yn-1-yl)-1H-indazole-6-carboxamide (Intermediate 24, 0.52 g, 1.75 mmol) in dimethylformamide (15 mL) was added 3-(6-bromopyridin-2-yl)-1-((2-(trimethylsilyl)ethoxy)methyl)piperidine-2,6-dione (Intermediate 276, 0.70 g, 1.75 mmol) followed by triethylamine (8.84 mL, 63.09 mmol). The reaction mixture was purged with argon for 15 min, then PdCl₂(PPh₃)₂ (0.12 g, 0.173 mmol) and copper (I) iodide (0.034 g, 0.17 mmol) were added. The reaction mixture was purged with argon for 10 min and then stirred at room temperature for 16 h. The reaction mixture was diluted with water (200 mL) and extracted with ethyl acetate (3×90 mL). The combined organic layers were separated, dried over anhydrous Na₂SO₄, and concentrated in vacuo. The crude material was purified by combi-flash column chromatography, by eluting with 80% ethyl acetate in heptane, to afford 1-(9-(6-(2,6-dioxo-1-((2-(trimethylsilyl)ethoxy)methyl)piperidin-3-yl) pyridin-2-yl)non-8-yn-1-yl)-3-methyl-1H-indazole-6-carboxamide (Intermediate 277, 0.55 g, 51%) as a yellow solid. ¹H NMR (400 MHz, DMSO-d₆) δ ppm −0.04 (s, 9H), 0.80-0.84 (m, 2H), 1.07-1.12 (m, 2H), 1.22-1.42 (m, 6H), 1.46-1.52 (m, 2H), 1.73-1.83 (m, 2H), 2.36-2.43 (m, 2H), 2.49 (s, 3H), 2.81-2.86 (m, 2H), 3.53 (t, J=7.60 Hz, 2H), 4.12-4.20 (m, 1H), 4.34 (t, J=6.40 Hz, 2H), 5.08 (s, 2H), 7.28-7.34 (m, 2H), 7.40 (br s, 1H), 7.54-7.65 (m, 1H), 7.70-7.78 (m, 2H), 8.02 (br s, 1H), 8.12 (s, 1H). Mass spec m/z 616.1 [M+H]⁺.

### Step 5

### Intermediate 278: tert-butyl 6-(1-(9-(6-(2,6-dioxo-1-((2-(trimethylsilyl)ethoxy)methyl)piperidin-3-yl)pyridin-2-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(9-(6-(2,6-dioxo-1-((2-(trimethylsilyl) ethoxy) methyl) piperidin-3-yl) pyridin-2-yl) non-8-yn-1-yl)-3-methyl-1H-indazole-6-carboxamide (Intermediate 277, 0.50 g, 0.81 mmol) in 1,4-dioxane (15 mL) was added tert-butyl-(R)-6-bromo-2-(1-methylpyrrolidin-2-yl)-1H-pyrrolo[3,2-c]pyridine-1-carboxylate (Intermediate 1, 0.37 g, 0.97 mmol) followed by cesium carbonate (0.79 g, 2.43 mmol). The reaction mixture was purged with argon for 15 min then Pd₂(dba)₃ (0.11 g, 0.12 mmol) and Xantphos (0.14 g, 0.24 mmol) were added. The reaction mixture was further purged with argon for 10 min and heated at 100° C. for 2 h. The reaction mixture was filtered through a celite bed and the filter pad was washed with ethyl acetate (100 mL). The filtrate was concentrated in vacuo and the crude material purified by combi-flash chromatography, by eluting with 90% ethyl acetate in heptane, to afford tert-butyl 6-(1-(9-(6-(2,6-dioxo-1-((2-(trimethylsilyl)ethoxy)methyl)piperidin-3-yl)pyridin-2-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 278, 0.54 g, 73%) as a yellow solid. ¹H NMR (401 MHz, DMSO-d₆) δ ppm −0.05 (s, 9H), 0.77-0.85 (m, 2H), 1.23-1.42 (m, 6H), 1.48-1.56 (m, 2H), 1.59-1.67 (m, 2H), 1.71 (s, 9H), 1.74-1.80 (m, 2H), 1.82-1.92 (m, 2H), 2.30-2.34 (m, 1H), 2.36 (s, 3H), 2.38-2.42 (m, 3H), 2.51 (s, 3H), 2.55-2.57 (m, 1H), 2.69-2.80 (m, 2H), 3.09-3.18 (m, 1H), 3.48-3.55 (m, 2H), 3.89-3.97 (m, 1H), 4.15-4.20 (m, 1H), 4.38-4.44 (m, 2H), 5.07 (s, 2H), 6.77 (s, 1H), 7.29-7.34 (m, 2H), 7.71-7.76 (m, 2H), 7.77-7.82 (m, 1H), 8.44 (s, 1H), 8.62 (s, 1H), 8.99 (s, 1H), 10.79 (s, 1H). Mass spec m/z 914.9 [M+H]⁺.

### Step 6

### Example 62: 1-(9-(6-(2,6-dioxopiperidin-3-yl) pyridin-2-yl) non-8-yn-1-yl)-3-methyl-N-(2-((R)-1-methylpyrrolidin-2-yl)-1H-pyrrolo [3,2-c]pyridin-6-yl)-1H-indazole-6-carboxamide

To a solution of tert-butyl 6-(1-(9-(6-(2,6-dioxo-1-((2-(trimethylsilyl)ethoxy)methyl)piperidin-3-yl)pyridin-2-yl)non-8-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 278, 0.54 g, 0.59 mmol) in acetonitrile (2 mL) was added methanesulfonic acid (0.39 mL, 5.90 mmol) and the reaction mixture was heated at 50° C. for 2 h. The reaction mixture was cooled to room temperature, then N,N′-dimethylethylenediamine (0.35 mL, 2.95 mmol) followed by triethylamine (1.65 mL, 11.80 mmol) were added and the mixture stirred at room temperature for 3 h. The reaction mixture was then concentrated in vacuo and the obtained residue was poured into water (50 mL). The resultant precipitate was collected by filtration and dried in vacuo. The crude material was purified by preparative HPLC (Method A) to afford 1-(9-(6-(2,6-dioxopiperidin-3-yl) pyridin-2-yl) non-8-yn-1-yl)-3-methyl-N-(2-((R)-1-methylpyrrolidin-2-yl)-1H-pyrrolo [3,2-c]pyridin-6-yl)-1H-indazole-6-carboxamide (Example 62, 0.074 g, 18%) as an off white solid. ¹H NMR (400 MHz, DMSO-d₆) δ ppm 1.27-1.43 (m, 6H), 1.48-1.57 (m, 2H), 1.78-1.96 (m, 5H), 2.05-2.12 (m, 1H), 2.13-2.16 (m, 1H), 2.18 (s, 3H), 2.22-2.31 (m, 2H), 2.40-2.44 (m, 2H), 2.51 (s, 3H), 2.53-2.56 (m, 2H), 3.12-3.18 (m, 1H), 3.32-3.34 (m, 1H), 3.98-4.02 (m, 1H), 4.38-4.44 (m, 2H), 6.41 (s, 1H), 7.28-7.33 (m, 2H), 7.70-7.76 (m, 2H), 7.77-7.81 (m, 1H), 8.26 (s, 1H), 8.44 (s, 1H), 8.52 (s, 1H), 10.54 (s, 1H), 10.88 (br s, 1H), 11.40 (br s, 1H). Mass spec m/z 685.2 [M+H]⁺.

### Example 63 was Synthesized Following Scheme 61

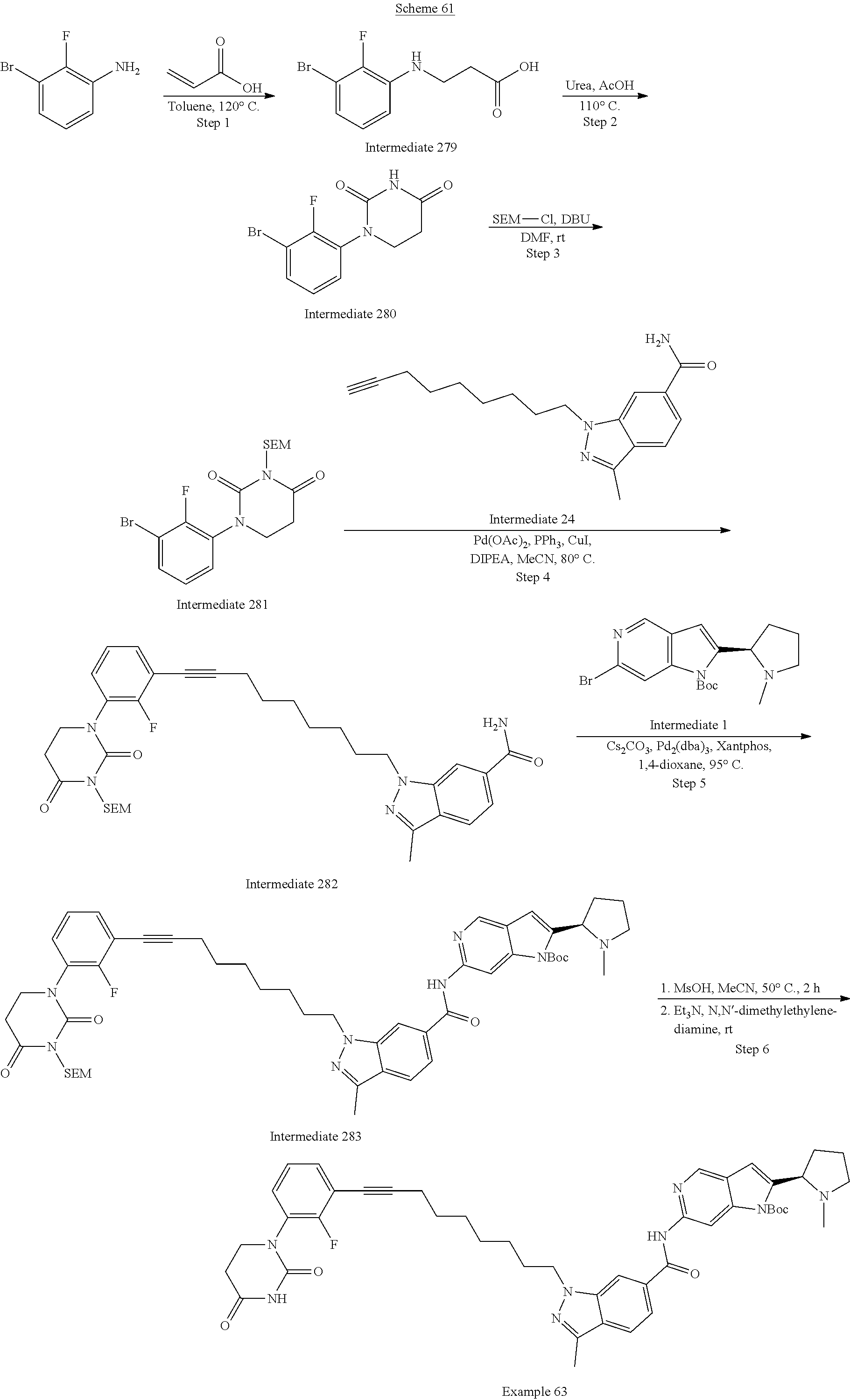

### Step 1

### Intermediate 279: 3-((3-bromo-2-fluorophenyl) amino) propanoic acid

To a solution of 3-bromo-2-fluoroaniline (CAS No. 58534-95-5, 2.00 g, 10.5 mmol) in toluene (20 mL) was added acrylic acid (CAS No. 79-10-7, 1.14 g, 15.8 mmol) at room temperature. The reaction mixture was heated at 120° C. for 18 h. The reaction mixture was concentrated in vacuo. The crude residue was poured into saturated NaHCO₃ solution (pH~9) and extracted with DCM (2×120 mL). The aqueous layer was acidified by adjusted to pH-5 with 1N HCl. The resultant precipitate was filtered, washed with heptane (60 mL) and then dried in vacuo to afford 3-((3-bromo-2-fluorophenyl) amino) propanoic acid (Intermediate 279, 2.00 g, 73%) as an off white solid which was used for the next step without further purification. ¹H NMR (400 MHz, DMSO-d₆) δ ppm 2.52-2.61 (m, 2H), 3.35-3.51 (m, 2H), 5.72 (br s, 1H), 6.67-6.85 (m, 2H), 6.86-6.98 (m, 1H), 12.25 (br s, 1H). Mass spec m/z 262.2 [M+H]⁺.

### Step 2

### Intermediate 280: 1-(3-bromo-2-fluorophenyl) dihydropyrimidine-2,4 (1H, 3H)-dione

A solution of 3-((3-bromo-2-fluorophenyl) amino) propanoic acid (Intermediate 279, 2.00 g, 7.6 mmol) in acetic acid (15 mL) was heated at 110° C. for 15 min then urea (2.3 g, 38 mmol) was added and the reaction mixture was stirred at 110° C. for 20 h. The reaction mixture was concentrated in vacuo. The obtained residue was poured into saturated NaHCO₃ solution (pH~9), the resultant precipitate collected by filtration, washed with heptane (50 mL) then dried in vacuo to afford 1-(3-bromo-2-fluorophenyl) dihydropyrimidine-2,4 (1H, 3H)-dione (Intermediate 280, 1.50 g, 68%) as an off white solid which was used for the next step without further purification. ¹H NMR (400 MHz, DMSO-d₆) δ ppm 2.72 (t, J=6.58 Hz, 2H), 3.74 (t, J=6.58 Hz, 2H), 7.22 (t, J=7.89 Hz, 1H), 7.47 (t, J=7.24 Hz, 1H), 7.66 (t, J=7.02 Hz, 1H), 10.53 (br s, 1H). Mass spec m/z 287.2 [M+H]⁺.

### Step 3

### Intermediate 281: 1-(3-bromo-2-fluorophenyl)-3-((2-(trimethylsilyl) ethoxy) methyl) dihydropyrimidine-2,4 (1H, 3H)-dione

To a solution of 1-(3-bromo-2-fluorophenyl) dihydropyrimidine-2,4 (1H, 3H)-dione (Intermediate 280, 1.40 g, 4.9 mmol) in dimethylformamide (20 mL) was added DBU (2.6 g, 17 mmol) at 0° C. followed by 2-(trimethylsilyl)methoxymethyl chloride (2.8 g, 17 mmol). The reaction mixture was stirred at room temperature for 16 h. The reaction mixture was poured into ice cold water (60 mL) and extracted with ethyl acetate (2×90 mL). The organic layer was separated, dried over anhydrous Na₂SO₄, filtered, and concentrated in vacuo. The crude material was purified by combi-flash chromatography, by eluting with 40% ethyl acetate in heptane, to afford 1-(3-bromo-2-fluorophenyl)-3-((2-(trimethylsilyl) ethoxy) methyl) dihydropyrimidine-2,4 (1H, 3H)-dione (Intermediate 281, 1.78 g, 87%) as a colourless gum. ¹H NMR (400 MHz, DMSO-d₆) δ ppm −0.01 (s, 9H), 0.85 (t, J=7.89 Hz, 2H), 2.91 (t, J=6.58 Hz, 2H), 3.56 (t, J=7.89 Hz, 2H), 3.76 (t, J=6.58 Hz, 2H), 5.11 (s, 2H), 7.25 (t, J=8.11 Hz, 1H), 7.49 (t, J=7.24 Hz, 1H), 7.70 (t, J=7.02 Hz, 1H).

### Step 4

### Intermediate 282: 1-(9-(3-(2,4-dioxo-3-((2-(trimethylsilyl) ethoxy) methyl) tetrahydropyrimidin-1 (2H)-yl)-2-fluorophenyl) non-8-yn-1-yl)-3-methyl-1H-indazole-6-carboxamide

To a solution of 1-(3-bromo-2-fluorophenyl)-3-((2-(trimethylsilyl) ethoxy) methyl) dihydropyrimidine-2,4 (1H, 3H)-dione (Intermediate 281, 1.47 g, 3.53 mmol) in acetonitrile (15 mL) was added 3-methyl-1-(non-8-yn-1-yl)-1H-indazole-6-carboxamide (Intermediate 24, 0.70 g, 2.35 mmol) followed by DIPEA (1.52 g, 11.77 mmol) at room temperature. The reaction mixture was purged with argon for 15 min followed the addition of triphenylphosphine (0.18 g, 0.70 mmol), copper (I) iodide (0.047 g, 0.23 mmol) and palladium acetate (0.058 g, 0.35 mmol). The reaction mixture was purged with argon for 10 min then heated at 80° C. for 16 h. The reaction mixture was concentrated in vacuo to obtain the crude compound. The crude material was purified by combi-flash column chromatography, by eluting with 10% MeOH in DCM, to afford 1-(9-(3-(2,4-dioxo-3-((2-(trimethylsilyl) ethoxy) methyl) tetrahydropyrimidin-1 (2H)-yl)-2-fluorophenyl) non-8-yn-1-yl)-3-methyl-1H-indazole-6-carboxamide (Intermediate 282, 0.44 g, 29%) as a yellow gum. ¹H NMR (400 MHz, DMSO-d₆) δ ppm −0.07 (s, 9H), 0.80 (t, J=7.89 Hz, 2H), 1.16-1.40 (m, 6H), 1.42-1.52 (m, 2H), 1.73-1.83 (m, 2H), 2.36-2.43 (m, 2H), 2.45 (s, 3H), 2.81-2.86 (m, 2H), 3.51 (t, J=7.67 Hz, 2H), 3.63-3.72 (m, 2H), 4.29 (t, J=6.80 Hz, 2H), 5.06 (s, 2H), 7.17 (t, J=7.60 Hz 1H), 7.34-7.42 (m, 3H), 7.55 (d, J=8.33 Hz, 1H), 7.68 (d, J=8.33 Hz, 1H), 7.98 (br s, 1H), 8.08 (s, 1H). Mass spec m/z 634.44 [M+H]⁺.

### Step 5

### Intermediate 283: tert-butyl (R)-6-(1-(9-(3-(2,4-dioxo-3-((2-(trimethylsilyl)ethoxy)methyl)tetrahydropyrimidin-1 (2H)-yl)-2-fluorophenyl)non-8-yn-1-yl)-3-methyl-1H-indazole-6-carboxamido)-2-(1-methylpyrrolidin-2-yl)-1H-pyrrolo[3,2-c]pyridine-1-carboxylate

To a solution of 1-(9-(3-(2,4-dioxo-3-((2-(trimethylsilyl) ethoxy) methyl) tetrahydropyrimidin-1 (2H)-yl)-2-fluorophenyl) non-8-yn-1-yl)-3-methyl-1H-indazole-6-carboxamide (Intermediate 282, 0.42 g, 0.67 mmol) in 1,4-dioxane (3 mL) was added tert-butyl (R)-6-bromo-2-(1-methylpyrrolidin-2-yl)-1H-pyrrolo[3,2-c]pyridine-1-carboxylate (Intermediate 1, 0.32 g, 0.84 mmol) followed by cesium carbonate (0.43 g, 1.26 mmol) at room temperature. The reaction mixture was purged with argon for 15 min followed by the addition of Pd₂(dba)₃ (0.11 g, 0.12 mmol) and Xantphos (0.076 g, 0.12 mmol). The reaction mixture was purged with argon for 10 min and then heated at 95° C. for 2 h. The reaction mixture was concentrated in vacuo to obtain crude compound. The crude material was purified by combi-flash column chromatography, by eluting with 10% MeOH in DCM, to afford tert-butyl (R)-6-(1-(9-(3-(2,4-dioxo-3-((2-(trimethylsilyl)ethoxy)methyl)tetrahydropyrimidin-1 (2H)-yl)-2-fluorophenyl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 283, 0.40 g, 51%) as an off white solid. ¹H NMR (400 MHz, DMSO-d₆) δ ppm −0.04 (s, 9H), 0.78-0.86 (m, 2H), 1.22-1.46 (m, 7H), 1.47-1.57 (m, 2H), 1.70 (s, 9H), 1.73-1.80 (m, 2H), 1.81-1.91 (m, 2H), 2.30-2.34 (m, 1H), 2.36 (s, 3H), 2.42-2.46 (m, 2H), 2.49 (s, 3H), 2.83-2.92 (m, 2H), 3.11-3.19 (m, 1H), 3.49-3.59 (m, 3H), 3.68-3.74 (m, 2H), 3.89-3.96 (m, 1H), 4.40 (t, J=6.80 Hz, 2H), 5.09 (s, 2H), 6.76 (s, 1H), 7.17-7.23 (m, 1H), 7.36-7.44 (m, 2H), 7.69-7.81 (m, 2H), 8.43 (s, 1H), 8.61 (s, 1H), 8.98 (s, 1H), 10.76 (s, 1H). Mass spec m/z 933.63 [M+H]⁺.

### Step 6

### Example 63: (R)-1-(9-(3-(2,4-dioxotetrahydropyrimidin-1 (2H)-yl)-2-fluorophenyl) non-8-yn-1-yl)-3-methyl-N-(2-(1-methylpyrrolidin-2-yl)-1H-pyrrolo [3,2-c]pyridin-6-yl)-1H-indazole-6-carboxamide

To a cooled (0° C.) solution of tert-butyl (R)-6-(1-(9-(3-(2,4-dioxo-3-((2-(trimethylsilyl)ethoxy)methyl)tetrahydropyrimidin-1 (2H)-yl)-2-fluorophenyl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 283, 0.30 g, 0.32 mmol) in acetonitrile (3 mL) was added methane sulfonic acid (0.32 g, 3.21 mmol) and the reaction mixture was then heated at 50° C. for 2 h. The reaction mixture was cooled to room temperature then N,N′-dimethylethylenediamine (0.18 g, 1.93 mmol), followed by triethylamine (0.68 g, 6.43 mmol), were added. The reaction was stirred at room temperature for 2 h. The reaction mixture was poured into ice cold water (50 mL), the resultant precipitate collected by filtration and dried in vacuo to afford the crude compound. The crude material was purified by preparative HPLC (Method A) to afford (R)-1-(9-(3-(2,4-dioxotetrahydropyrimidin-1 (2H)-yl)-2-fluorophenyl) non-8-yn-1-yl)-3-methyl-N-(2-(1-methylpyrrolidin-2-yl)-1H-pyrrolo [3,2-c]pyridin-6-yl)-1H-indazole-6-carboxamide (Example 63, 0.075 g, 23%) as an off white solid. ¹H NMR (400 MHz, DMSO-d₆) δ ppm 1.34-1.48 (m, 7H), 1.56-1.62 (m, 2H), 1.90-1.97 (m, 5H), 2.31-2.33 (m, 1H), 2.35 (s, 3H), 2.38-2.40 (m, 1H), 2.52 (s, 3H), 2.54-2.56 (m, 1H), 2.73-2.79 (m, 2H), 3.19-3.24 (m, 1H), 3.38-3.42 (m, 1H), 3.77 (t, J=6.80 Hz, 2H), 4.47 (t, J=7.00 Hz, 2H), 6.47 (s, 1H), 7.25 (t, J=7.80 Hz, 1H), 7.42-7.49 (m, 2H), 7.79-7.86 (m, 2H), 8.32 (s, 1H), 8.51 (s, 1H), 8.59 (s, 1H), 10.58 (s, 1H), 10.60 (s, 1H). Mass spec m/z 703.10 [M+H]⁺.

### Example 64 was Synthesized Following Scheme 62

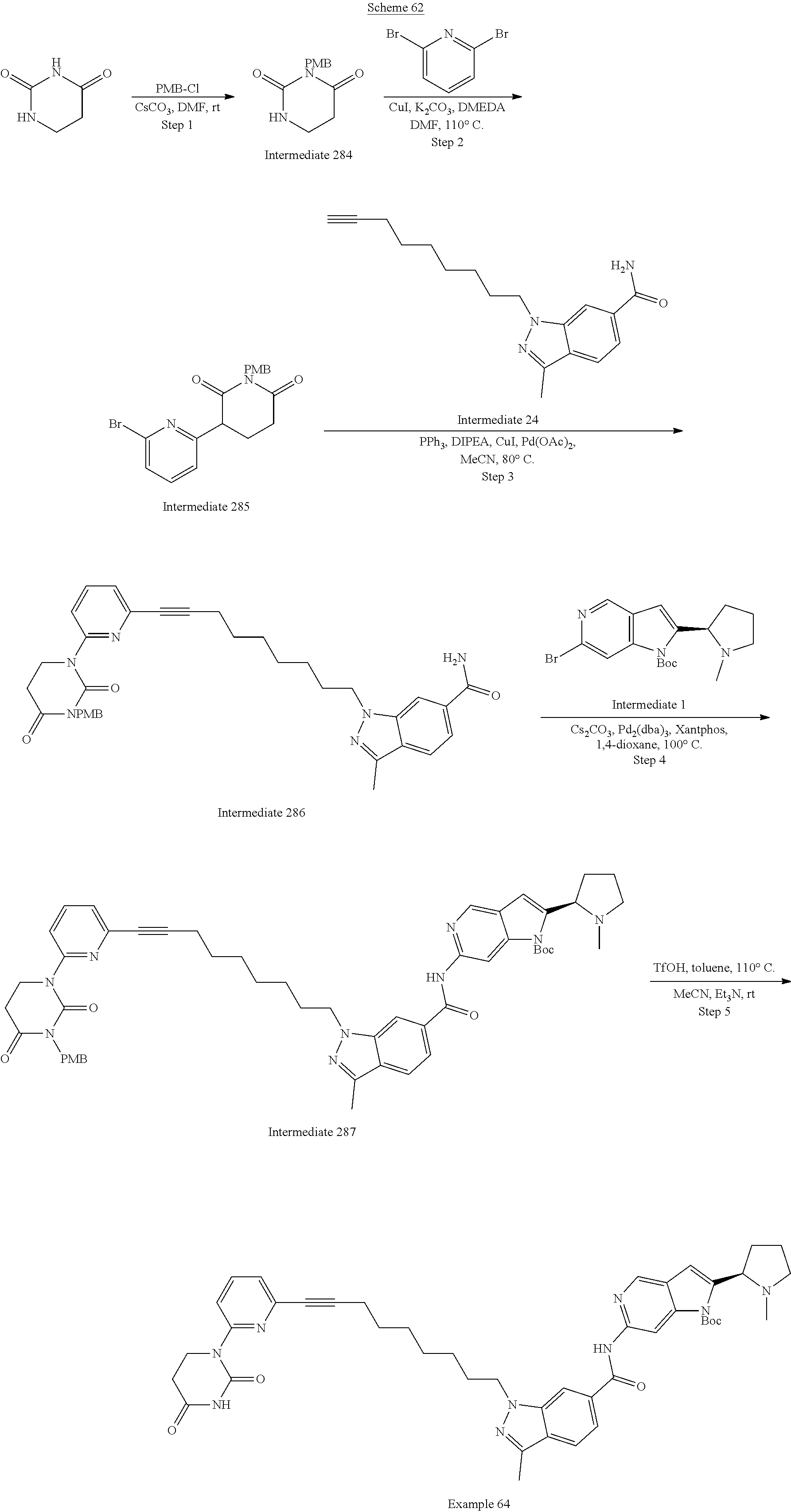

### Step 1

### Intermediate 284: 3-(4-methoxybenzyl) dihydropyrimidine-2,4 (1H, 3H)-dione

To a solution of dihydropyrimidine-2,4 (1H,3H)-dione (CAS No: 504-07-4, 5.00 g, 43.82 mmol) in dimethylformamide (150 mL) was added cesium carbonate (28.56 g, 87.64 mmol) followed by 4-methoxybenzyl chloride (CAS No: 824-94-2, 4.06 mL, 28.48 mmol). The reaction mixture was stirred at room temperature for 16 h. The reaction mixture was quenched with ice cold water (1500 mL) and stirred for 30 min, the resultant precipitate collected by filtration and dried in vacuo to afford 3-(4-methoxybenzyl) dihydropyrimidine-2,4 (1H,3H)-dione (Intermediate 284, 4.50 g, 44%) as an off white solid, which was used for the next step without further purification. ¹H NMR (400 MHz, DMSO-d₆) δ ppm 2.62 (t, J=6.80 Hz, 2H), 3.19-3.23 (m, 2H), 3.71 (s, 3H), 4.71 (s, 2H), 6.83-6.85 (m, 2H), 7.16-7.18 (m, 2H), 7.80 (br s, 1H).

### Step 2

### Intermediate 285: 1-(6-bromopyridin-2-yl)-3-(4-methoxybenzyl) dihydropyrimidine-2,4 (1H,3H)-dione

To a solution of 2,6-dibromopyridine (CAS No: 626-05-1, 7.00 g, 29.5 mmol) in dimethylformamide (10 mL) was added 3-(4-methoxybenzyl)dihydropyrimidine-2,4 (1H, 3H1)-dione (Intermediate 284, 4.15 g, 17.7 mmol) followed by K₂CO₃ (12.3 g, 88.6 mmol). The reaction mixture was purged with argon gas for 15 min then copper (1) iodide (0.56 g, 2.95 mmol) and N,N′-dimethylethylenediamine (0.53 g, 5.91 mmol) were added. The reaction mixture was heated at 110° C. for 16 h. The reaction mixture was filtered and the precipitate washed with ethyl acetate (100 mL). The filtrate was washed with ice cold water (100 mL). The organic layer was separated, dried over Na₂SO₄, and concentrated in vacuo. The crude material was purified by combi-flash column chromatography, by eluting with 20% ethyl acetate in heptane, to afford 1-(6-bromopyridin-2-yl)-3-(4-methoxybenzyl) dihydropyrimidine-2,4 (1H,3H)-dione (Intermediate 285, 3.4 g, 29%) as an off white solid. ¹H NMR (400 MHz, DMSO-d₆) δ ppm 2.88 (t, J=6.80 Hz, 2H), 3.72 (s, 3H), 4.02 (t, J=6.80 Hz, 2H), 4.83 (s, 2H), 6.84-6.86 (m, 2H), 7.22-7.25 (m, 2H), 7.42-7.47 (m, 1H), 7.76-.7.79 (m, 2H).

### Step 3

### Intermediate 286: 1-(9-(6-(3-(4-methoxybenzyl)-2,4-dioxotetrahydropyrimidin-1 (2H)-yl)pyridin-2-yl)non-8-yn-1-yl)-3-methyl-1H-indazole-6-carboxamide

To a solution of 1-(6-bromopyridin-2-yl)-3-(4-methoxybenzyl)dihydropyrimidine-2,4 (1H,3H)-dione (Intermediate 285, 1.5 g, 3.8 mmol) in acetonitrile (25 mL) was added 3-methyl-1-(non-8-yn-1-yl)-1H-indazole-6-carboxamide (Intermediate 24, 3.8 mmol, 5.8 mmol) followed by DIPEA (3.3 mL, 19 mmol) at room temperature. The reaction mixture was purged with argon gas for 15 min then triphenylphosphine (0.30 g, 1.2 mmol), copper (I) iodide (0.07 g, 0.38 mmol) and palladium (II) acetate (0.095 g, 0.58 mmol) were added. The reaction mixture was further purged with argon gas for another 10 min and then heated at 80° C. for 16 h. The reaction mixture was concentrated in vacuo to obtain crude compound. The crude material was purified by combi-flash chromatography, by eluting with 15% ethyl acetate in heptane, to afford 1-(9-(6-(3-(4-methoxybenzyl)-2,4-dioxotetrahydropyrimidin-1 (2H)-yl)pyridin-2-yl)non-8-yn-1-yl)-3-methyl-1H-indazole-6-carboxamide (Intermediate 286, 1.5 g, 64%) as a yellow gummy liquid. ¹H NMR (400 MHz, DMSO-d₆) δ ppm 1.21-1.42 (m, 6H), 1.46-1.56 (m, 2H), 1.76-1.87 (m, 2H), 2.40 (t, J=7.20 Hz, 2H), 2.48 (s, 3H), 2.85 (t, J=6.80 Hz, 2H), 3.71 (s, 3H), 3.99 (t, J=6.80 Hz, 2H), 4.33 (t, J=7.20 Hz, 2H), 4.83 (s, 2H), 6.84-6.87 (m, 2H), 7.22-7.26 (m, 3H), 7.41 (br s, 1H), 7.56-7.66 (m, 2H), 7.69-7.80 (m, 2H), 8.03 (br s, 1H), 8.12 (s, 1H). Mass spec m/z 607.38 [M+H]⁺.

### Step 4

### Intermediate 287: tert-butyl (R)-6-(1-(9-(6-(3-(4-methoxybenzyl)-2,4-dioxotetrahydropyrimidin-1 (2H)-yl)pyridin-2-yl)non-8-yn-1-yl)-3-methyl-1H-indazole-6-carboxamido)-2-(1-methylpyrrolidin-2-yl)-1H-pyrrolo[3,2-c]pyridine-1-carboxylate

To a solution of 1-(9-(6-(3-(4-methoxybenzyl)-2,4-dioxotetrahydropyrimidin-1 (2H)-yl)pyridin-2-yl)non-8-yn-1-yl)-3-methyl-1H-indazole-6-carboxamide (Intermediate 286, 0.70 g, 1.15 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 1, 0.48 g, 1.27 mmol) followed by cesium carbonate (1.14 g, 3.46 mmol) at room temperature. The reaction mixture was purged with argon for 15 min followed by the addition of Pd₂(dba)₃ (0.16 g, 0.17 mmol) and Xantphos (0.20 g, 0.34 mmol). The reaction mixture was purged with argon for 10 min and then heated at 100° C. for 2 h. The reaction mixture was concentrated in vacuo to obtain crude compound. The crude material was purified by combi-flash chromatography, by eluting with 3% MeOH in DCM, to afford tert-butyl (R)-6-(1-(9-(6-(3-(4-methoxybenzyl)-2,4-dioxotetrahydropyrimidin-1 (2H)-yl)pyridin-2-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 287, 0.6 g, 57%) as off brown solid. ¹H NMR (400 MHz, DMSO-d₆) δ ppm 1.21-1.42 (m, 6H), 1.47-1.56 (m, 2H), 1.59-1.66 (m, 1H), 1.70 (s, 9H), 1.82-1.92 (m, 3H), 2.35 (s, 3H), 2.45 (s, 3H), 2.82-2.86 (m, 2H), 3.13-3.18 (m, 3H), 3.36-3.56 (m, 3H), 3.71 (s, 3H), 3.89-4.02 (m, 3H), 4.06-4.10 (m, 2H), 4.38-4.42 (m, 2H), 6.76 (s, 1H), 6.83-6.86 (m, 2H), 7.17-7.26 (m, 3H), 7.61-7.64 (m, 1H), 7.68-7.81 (m, 3H), 8.43 (s, 1H), 8.60 (s, 1H), 8.98 (s, 1H), 10.75 (br s, 1H). Mass spec m/z 906.57 [M+H]⁺.

### Step 5

### Example 64: (R)-1-(9-(6-(2,4-dioxotetrahydropyrimidin-1 (2H)-yl)pyridin-2-yl)non-8-yn-1-yl)-3-methyl-N-(2-(1-methylpyrrolidin-2-yl)-1H-pyrrolo[3,2-c]pyridin-6-yl)-1H-indazole-6-carboxamide

To a solution of tert-butyl (R)-6-(1-(9-(6-(3-(4-methoxybenzyl)-2,4-dioxotetrahydropyrimidin-1 (2H)-yl)pyridin-2-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 287, 0.50 g, 0.55 mmol) in toluene (3 mL) was added trifluoromethanesulfonic acid (0.49 mL, 5.52 mmol) then the reaction was heated to 110° C. for 3 h. The toluene layer was decanted and the resulting residue was taken up in acetonitrile (10 mL). Triethylamine (1.55 mL, 11.04 mmol) was added and the reaction was stirred at room temperature for 10 min then concentrated in vacuo. The obtained residue was diluted with ice cold water (50 mL), the resultant precipitate collected by filtration and dried in vacuo to afford the crude compound. The crude material was purified by preparative HPLC (Method A) to afford (R)-1-(9-(6-(2,4-dioxotetrahydropyrimidin-1 (2H)-yl)pyridin-2-yl)non-8-yn-1-yl)-3-methyl-N-(2-(1-methylpyrrolidin-2-yl)-1H-pyrrolo[3,2-c]pyridin-6-yl)-1H-indazole-6-carboxamide (Example 64, 0.039 g, 10%) as an off white solid. ¹H NMR (400 MHz, DMSO-d₆) δ ppm 1.23-1.43 (m, 7H), 1.48-1.57 (m, 2H), 1.76-1.97 (m, 5H), 2.12-2.16 (m, 1H), 2.17 (s, 3H), 2.24-2.30 (m, 1H), 2.40-2.44 (m, 2H), 2.52 (s, 3H), 2.63-2.67 (m, 2H), 3.12-3.18 (m, 1H), 3.98-4.02 (m, 2H), 4.38-4.42 (m, 2H), 6.40 (s, 1H), 7.18-7.20 (m, 1H), 7.66-7.81 (m, 4H), 8.25 (s, 1H), 8.43 (s, 1H), 8.52 (s, 1H), 10.52 (br s, 2H), 11.38 (br s, 1H). Mass spec m/z 686.0 [M+H]⁺.

### Example 65 was Synthesized Following Scheme 63

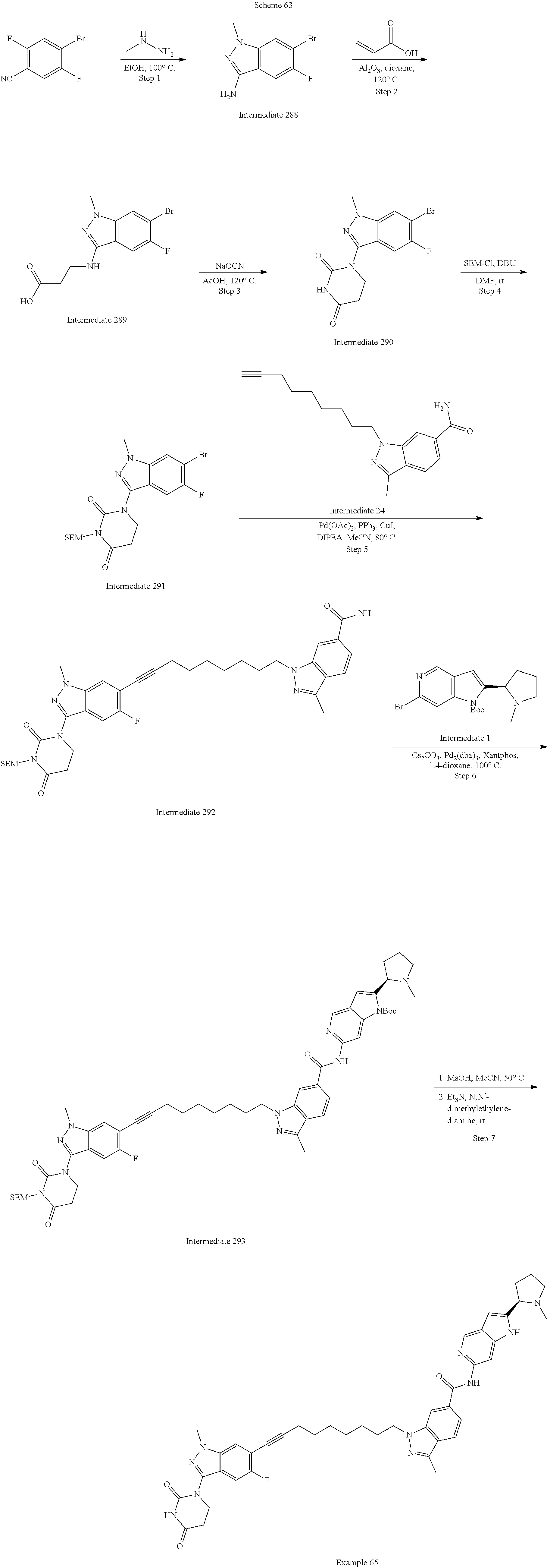

### Step 1

### Intermediate 288: 6-bromo-5-fluoro-1-methyl-1H-indazol-3-amine

To a solution of 4-bromo-2,5-difluorobenzonitrile (CAS No: 133541-45-4, 5.00 g, 22.93 mmol) in ethanol (50 mL) was added methyl hydrazine (5.28 g, 114.68 mmol) and the reaction mixture was heated at 100° C. for 16 h. The reaction mixture was concentrated in vacuo and the resultant residue was diluted with water (300 mL). The resultant precipitate was collected by filtration and dried in vacuo to afford 6-bromo-5-fluoro-1-methyl-1H-indazol-3-amine (Intermediate 288, 3.00 g, 54%) as a white solid, which was used for the next step without further purification. ¹H NMR (400 MHz, DMSO-d) δ ppm 3.71 (s, 3H), 5.51 (s, 2H), 7.62 (d, J=9.21 Hz, 1H), 7.81 (d, J=5.70 Hz, 1H). Mass spec m/z 244.2 [M+H]⁺.

### Step 2

### Intermediate 289: 3-((6-bromo-5-fluoro-1-methyl-1H-indazol-3-yl) amino) propanoic acid

To a solution of 6-bromo-5-fluoro-1-methyl-1H-indazol-3-amine (Intermediate 288, 3.00 g, 12.3 mmol) in 1,4-dioxane (30 mL) was added acrylic acid (CAS No: 79-10-7, 2.66 g, 36.9 mmol) followed by aluminium oxide (1.5 mL, 49.2 mmol) and the reaction mixture was heated at 120° C. for 16 h. The reaction mixture was filtered through a celite bed and the filter pad was washed with dichloromethane (50 mL). The filtrate was concentrated in vacuo and the resultant crude material purified by combi-flash chromatography, by eluting with 50% ethyl acetate in heptane, to afford 3-((6-bromo-5-fluoro-1-methyl-1H-indazol-3-yl)amino)propanoic acid (Intermediate 289, 2.30 g, 52%) as an off-white solid. ¹H NMR (400 MHz, DMSO-d₆) δ ppm 2.60 (t, J=6.40 Hz, 2H), 3.44 (t, J=6.40 Hz, 2H), 3.75 (s, 3H), 6.16-6.25 (m, 1H), 7.65 (d, J=9.20 Hz, 1H), 7.82 (d, J=5.60 Hz, 1H), 12.19 (br s, 1H). Mass spec m/z 316.2 [M+H]⁺.

### Step 3

### Intermediate 290: 1-(6-bromo-5-fluoro-1-methyl-1H-indazol-3-yl)dihydropyrimidine-2,4 (1H, 3H)-dione

To a solution of 3-((6-bromo-5-fluoro-1-methyl-1H-indazol-3-yl) amino)propanoic acid (Intermediate 289, 1.90 g, 6.0 mmol) in acetic acid (20 mL) was added sodium cyanate (0.88 g, 18 mmol) and the reaction mixture was heated at 120° C. for 48 h. The reaction mixture was concentrated in vacuo to afford crude 1-(6-bromo-5-fluoro-1-methyl-1H-indazol-3-yl) dihydropyrimidine-2,4 (1H,3H)-dione (Intermediate 290, 0.60 g, 27%) as a grey solid, which was used for the next step without further purification. ¹H NMR (400 MHz, DMSO-d₆) δ ppm 2.78 (t, J=6.40 Hz, 2H), 3.94 (t, J=6.40 Hz, 2H), 4.02 (s, 3H), 7.63 (d, J=9.20 Hz, 1H), 8.18 (d, J=5.60 Hz, 1H), 10.61 (s, 1H). Mass spec m/z 341.08 [M+H]⁺.

### Step 4

### Intermediate 291: 1-(6-bromo-5-fluoro-1-methyl-1H-indazol-3-yl)-3-((2-(trimethylsilyl)ethoxy)methyl) dihydropyrimidine-2,4 (1H, 3H)-dione

To a cooled (0° C.) solution of 1-(6-bromo-5-fluoro-1-methyl-1H-indazol-3-yl)dihydropyrimidine-2,4 (1H,3H)-dione (Intermediate 290, 1.00 g, 2.93 mmol) in dimethylformamide (4 mL) was added DBU (1.35 mL, 8.79 mmol) followed by 2-(trimethylsilyl)ethoxymethyl chloride (1.1 mL, 5.86 mmol). The reaction mixture was stirred at room temperature for 16 h. The reaction mixture was diluted with water (200 mL) and extracted with ethyl acetate (3×100 mL). The combined organic layers were separated, dried over anhydrous Na₂SO₄, filtered, and concentrated in vacuo. The crude material was purified by combi-flash chromatography, by eluting with 50% ethyl acetate in heptane, to afford 1-(6-bromo-5-fluoro-1-methyl-1H-indazol-3-yl)-3-((2-(trimethylsilyl)ethoxy) methyl)dihydropyrimidine-2,4 (1H, 3H)-dione (Intermediate 291, 0.90 g, 65%) as a yellow liquid. ¹H NMR (400 MHz, DMSO-d₆) δ ppm −0.01 (s, 9H), 0.84-0.88 (m, 2H), 2.92-2.96 (m, 2H), 3.56-3.62 (m, 2H), 3.91-3.94 (m, 2H), 4.01 (s, 3H), 5.15 (s, 2H), 7.56 (d, J=8.7 Hz, 1H), 8.19 (d, J=5.8 Hz, 1H).

### Step 5

### Intermediate 292: 1-(9-(3-(2,4-dioxo-3-((2-(trimethylsilyl)ethoxy)methyl)tetrahydropyrimidin-1 (2H)-yl)-5-fluoro-1-methyl-1H-indazol-6-yl)non-8-yn-1-yl)-3-methyl-1H-indazole-6-carboxamide

To a solution of 3-methyl-1-(non-8-yn-1-yl)-1H-indazole-6-carboxamide (Intermediate 24, 0.63 g, 2.1 mmol) in acetonitrile (20 mL) was added 1-(6-bromo-5-fluoro-1-methyl-1H-indazol-3-yl)-3-((2-(trimethylsilyl)ethoxy) methyl)dihydropyrimidine-2,4 (1H,3H)-dione (Intermediate 291, 1.00 g, 2.1 mmol) followed by DIPEA (2 mL, 11 mmol). The reaction mixture was purged with argon for 15 min, then copper (I) iodide (0.043 g, 0.21 mmol), Pd(OAc)₂ (0.053 g, 0.32 mmol) and triphenylphosphine (0.17 g, 0.64 mmol) were added. The reaction mixture was further purged with argon for 10 min and then heated at 80° C. for 4 h. The reaction mixture was diluted with water (100 mL) and extracted with ethyl acetate (3×50 mL). The combined organic layers were separated, dried over anhydrous Na₂SO₄, filtered, and concentrated in vacuo. The crude material was purified by combi-flash chromatography, by eluting with 2% MeOH in dichloromethane, to afford 1-(9-(3-(2,4-dioxo-3-((2-(trimethylsilyl)ethoxymethyl)tetrahydropyrimidin-1 (2H)-yl)-5-fluoro-1-methyl-1H-indazol-6-yl)non-8-yn-1-yl)-3-methyl-1H-indazole-6-carboxamide (Intermediate 292, 0.80 g, 47%) as a yellow solid. ¹H NMR (400 MHz, DMSO-d₆) δ ppm −0.01 (s, 9H), 0.84-0.88 (m, 2H), 1.23-1.46 (m, 7H), 1.52-1.58 (m, 2H), 1.78-1.88 (m, 2H), 2.47 (s, 3H), 2.52-2.57 (m, 1H), 2.92-2.96 (m, 2H), 3.57-3.62 (m, 2H), 3.90-3.94 (m, 2H), 3.99 (s, 3H), 4.32-4.36 (m, 2H), 5.15 (s, 2H), 7.38-7.42 (m, 2H), 7.59 (d, J=8.80 Hz, 1H), 7.72 (d, J=8.80 Hz, 1H), 7.82-7.84 (m, 1H), 8.03 (br s, 1H), 8.12 (s, 1H). Mass spec m/z 688.3 [M+H]⁺.

### Step 6

### Intermediate 293: tert-butyl-(R)-6-(1-(9-(3-(2,4-dioxo-3-((2-(trimethylsilyl)ethoxy)methyl)tetrahydropyrimidin-1 (2H)-yl)-5-fluoro-1-methyl-1H-indazol-6-yl)non-8-yn-1-yl)-3-methyl-1H-indazole-6-carboxamido)-2-(1-methylpyrrolidin-2-yl)-1H-pyrrolo[3,2-c]pyridine-1-carboxylate

To a solution of 1-(9-(3-(2,4-dioxo-3-((2-(trimethylsilyl) ethoxy)methyl)tetrahydropyrimidin-1 (2H)-yl)-5-fluoro-1-methyl-1H-indazol-6-yl) non-8-yn-1-yl)-3-methyl-1H-indazole-6-carboxamide (Intermediate 292, 0.50 g, 0.73 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 1, 0.30 g, 0.79 mmol) followed by cesium carbonate (0.35 g, 1.82 mmol). The reaction mixture was purged with argon for 15 min then Pd₂(dba)₃ (0.10 g, 0.11 mmol) and Xantphos (0.13 g, 0.22 mmol) were added. The reaction mixture was further purged with argon for 10 min and then heated at 100° C. for 2 h. The reaction mixture was concentrated in vacuo and the crude material was purified by combi-flash chromatography, by eluting with 70% ethyl acetate in heptane, to afford tert-butyl (R)-6-(1-(9-(3-(2,4-dioxo-3-((2-(trimethylsilyl)ethoxy)methyl)tetrahydropyrimidin-1 (2H)-yl)-5-fluoro-1-methyl-1H-indazol-6-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 293, 0.56 g, 78%) as a yellow solid. ¹H NMR (400 MHz, DMSO-d₆) δ ppm −0.02 (s, 9H), 0.82-0.90 (m, 2H), 1.25-1.48 (m, 6H), 1.51-1.62 (m, 2H), 1.70 (s, 9H), 1.73-1.78 (m, 2H), 1.85-1.88 (m, 3H), 2.32-2.34 (m, 1H), 2.35 (s, 3H), 2.49 (s, 3H), 2.53-2.56 (m, 2H), 2.91-2.94 (m, 2H), 3.12-3.16 (m, 1H), 3.56-3.60 (m, 3H), 3.90-3.94 (m, 3H), 3.97 (s, 3H), 4.40-4.43 (m, 2H), 5.14 (s, 2H), 6.75 (s, 1H), 7.35-7.43 (m, 1H), 7.69-7.74 (m, 1H), 7.76-7.84 (m, 2H), 8.43 (s, 1H), 8.59 (s, 1H), 8.97 (s, 1H), 10.76 (br s, 1H).

### Step 7

### Example 65: (R)-1-(9-(3-(2,4-dioxotetrahydropyrimidin-1 (2H)-yl)-5-fluoro-1-methyl-1H-indazol-6-yl)non-8-yn-1-yl)-3-methyl-N-(2-(1-methylpyrrolidin-2-yl)-1H-pyrrolo[3,2-c]pyridin-6-yl)-1H-indazole-6-carboxamide

To a solution of tert-butyl-(R)-6-(1-(9-(3-(2,4-dioxo-3-((2-(trimethylsilyl)ethoxy)methyl)tetrahydropyrimidin-1 (2H)-yl)-5-fluoro-1-methyl-1H-indazol-6-yl)non-8-yn-1-yl)-3-methyl-1H-indazole-6-carboxamido)-2-(1-methylpyrrolidin-2-yl)-1H-pyrrolo[3,2-c]pyridine-1-carboxylate (Intermediate 293, 0.56 g, 0.57 mmol) in acetonitrile (15 mL) was added methanesulfonic acid (0.37 mL, 5.67 mmol) and the reaction mixture was heated at 50° C. for 2 h. The reaction mixture was cooled to room temperature, then N,N′-dimethylethylenediamine (0.34 mL, 2.83 mmol) followed by triethylamine (1.59 mL, 11.35 mmol) were added and the mixture stirred at room temperature for 3 h then concentrated in vacuo. The obtained residue was diluted with water (50 mL) and the resultant precipitate was collected by filtration and dried in vacuo. The crude material was purified by preparative HPLC (Method A) to afford (R)-1-(9-(3-(2,4-dioxotetrahydropyrimidin-1 (2H)-yl)-5-fluoro-1-methyl-1H-indazol-6-yl)non-8-yn-1-yl)-3-methyl-N-(2-(1-methylpyrrolidin-2-yl)-1H-pyrrolo[3,2-c]pyridin-6-yl)-1H-indazole-6-carboxamide (Example 65, 0.075 g, 17%) as an off-white solid. ¹H NMR (400 MHz, DMSO-d₆) δ ppm 1.27-1.48 (m, 7H), 1.51-1.60 (m, 2H), 1.82-1.95 (m, 5H), 2.12-2.16 (m, 1H), 2.18 (s, 3H), 2.24-2.30 (m, 1H), 2.51 (s, 3H), 2.73-2.77 (m, 2H), 3.12-3.18 (m, 1H), 3.34-3.45 (m, 2H), 3.88-3.92 (m, 2H), 3.96 (s, 3H), 4.38-4.44 (m, 2H), 6.40 (s, 1H), 7.43-7.45 (m, 1H), 7.70-7.83 (m, 3H), 8.25 (s, 1H), 8.44 (s, 1H), 8.51 (s, 1H), 10.52 (s, 1H), 10.57 (s, 1H), 11.38 (s, 1H). Mass spec m/z 756.9 [M+H]⁺.

### Example 66 was Synthesized Following Scheme 64

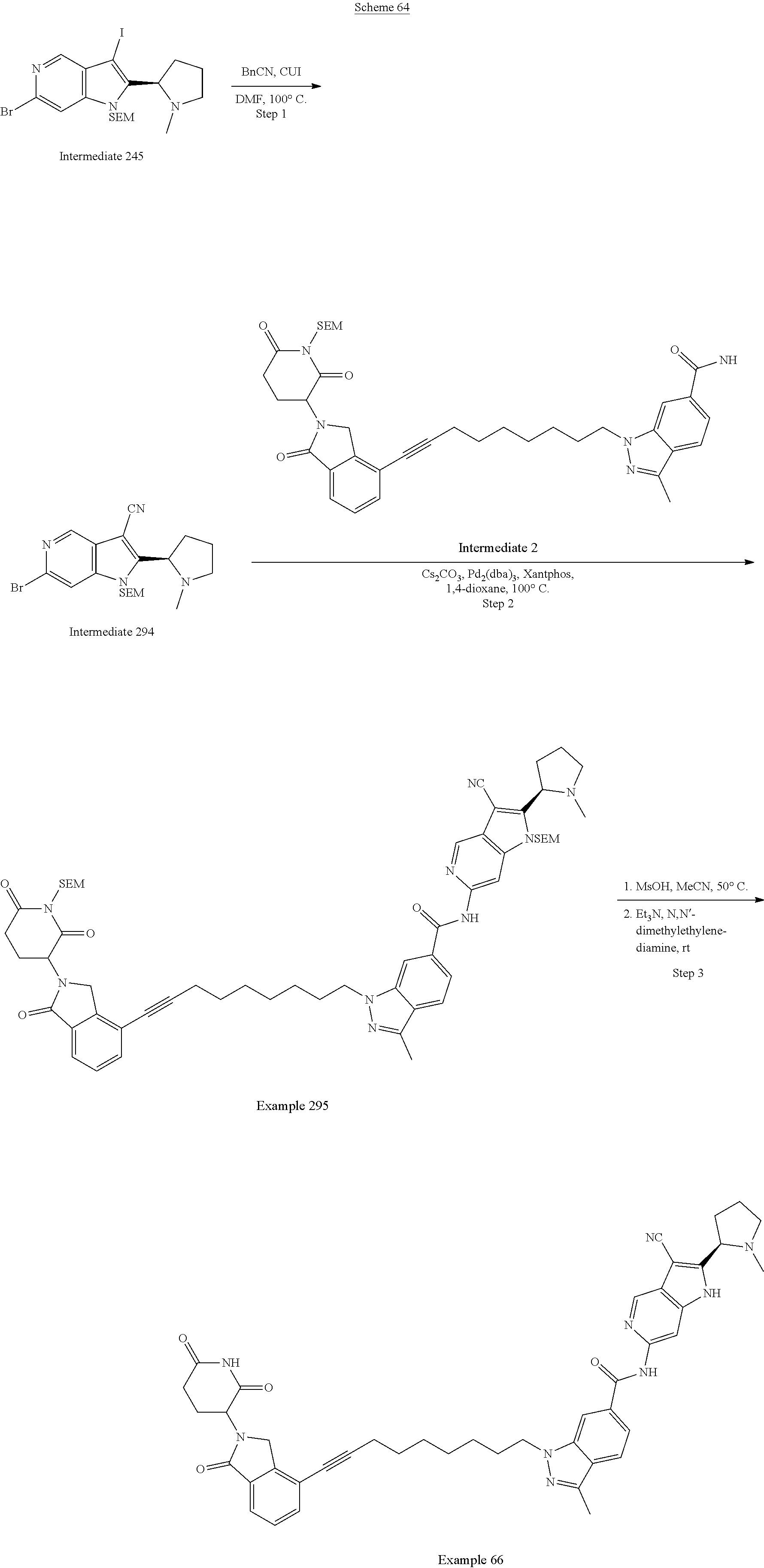 [SMILES: CN1CCC[C@@H]1c1c(I)c2cnc(Br)cc2n1COCC[Si](C)(C)C ; CN1CCC[C@@H]1c1c(C#N)c2cnc(Br)cc2n1COCC[Si](C)(C)C ; Cc1nn(CCCCCCCC#Cc2cccc3c2CN(C2CCC(=O)N(COCC[Si](C)(C)C)C2=O)C3=O)c2cc(C(N)=O)ccc12 ; Cc1nn(CCCCCCCC#Cc2cccc3c2CN(C2CCC(=O)N(COCC[Si](C)(C)C)C2=O)C3=O)c2cc(C(=O)Nc3cc4c(cn3)c(C#N)c([C@H]3CCCN3C)n4COCC[Si](C)(C)C)ccc12 ; Cc1nn(CCCCCCCC#Cc2cccc3c2CN(C2CCC(=O)NC2=O)C3=O)c2cc(C(=O)Nc3cc4[nH]c([C@H]5CCCN5C)c(C#N)c4cn3)ccc12]

### Step 1

### Intermediate 294: (R)-6-bromo-2-(1-methylpyrrolidin-2-yl)-1-((2-(trimethylsilyl)ethoxy)methyl)-1H-pyrrolo [3,2-c]pyridine-3-carbonitrile

To a solution of (R)-6-bromo-3-iodo-2-(1-methylpyrrolidin-2-yl)-1-((2-(trimethylsilyl)ethoxy)methyl)-1H-pyrrolo[3,2-c]pyridine (Intermediate 245, 1.00 g, 1.9 mmol) in dimethylformamide (10 mL) was added copper (I) iodide (0.43 g, 2.2 mmol) and benzyl cyanide (1.8 g, 15.0 mmol). The reaction mixture was heated at 100° C. for 16 h. The reaction mixture was diluted with water (100 mL) and extracted with ethyl acetate (3×100 mL). The organic layer was separated, dried over anhydrous Na₂SO₄, filtered, and concentrated in vacuo. The crude material was purified by combi-flash chromatography, by eluting with 30% ethyl acetate in heptane, to afford (R)-6-bromo-2-(1-methylpyrrolidin-2-yl)-1-((2-(trimethylsilyl)ethoxy)methyl)-1H-pyrrolo[3,2-c]pyridine-3-carbonitrile (Intermediate 294, 0.2 g, 20%) as a yellow liquid. ¹H NMR (400 MHz, DMSO-d₆) δ ppm −0.08 (s, 9H), 0.81-0.84 (m, 2H), 1.80-1.97 (m, 3H), 2.26 (s, 3H), 2.34-2.36 (m, 2H), 3.16-3.22 (m, 1H), 3.51-3.59 (m, 2H), 3.78-3.82 (m, 1H), 5.74-5.78 (m, 2H), 8.12 (s, 1H), 8.68 (s, 1H). Mass spec m/z 435.1 [M+H]⁺.

### Step 2

### Intermediate 295: N-(3-cyano-2-((R)-1-methylpyrrolidin-2-yl)-1-((2-(trimethylsilyl)ethoxy)methyl)-1H-pyrrolo[3,2-c]pyridin-6-yl)-1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide

To a solution of (R)-6-bromo-2-(1-methylpyrrolidin-2-yl)-1-((2-(trimethylsilyl)ethoxy)methyl)-1H-pyrrolo[3,2-c]pyridine-3-carbonitrile (Intermediate 294, 0.25 g, 0.57 mmol) in 1,4-dioxane (3 mL) was added 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 2, 0.30 g, 0.45 mmol) followed by cesium carbonate (0.56 g, 1.72 mmol) at room temperature. The reaction mixture was purged with argon for 15 min followed by addition of Pd₂(dba)₃ (0.10 g, 0.11 mmol) and Xantphos (0.06 g, 0.11 mmol). The reaction mixture was further purged with argon for another 10 min and heated at 100° C. for 2 h. The reaction mixture was filtered through a celite bed and washed with ethyl acetate (100 mL). The filtrate was concentrated in vacuo. The crude material was washed with heptane and dried in vacuo to afford N-(3-cyano-2-((R)-1-methylpyrrolidin-2-yl)-1-((2-(trimethylsilyl)ethoxy)methyl)-1H-pyrrolo[3,2-c]pyridin-6-yl)-1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 295, 0.23 g) as yellow solid, which was carried forward into next step without further purification. Mass spec m/z 1023.6 [M+H]⁺.

### Step 3

### Example 66: N-(3-cyano-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide

To a cooled (0° C.) solution of N-(3-cyano-2-((R)-1-methylpyrrolidin-2-yl)-1-((2-(trimethylsilyl)ethoxy)methyl)-1H-pyrrolo[3,2-c]pyridin-6-yl)-1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 295, 0.23 g, 0.22 mmol) in acetonitrile (2 mL) was added methanesulfonic acid (0.22 mL, 3.37 mmol) and the reaction then heated at 50° C. for 2h. The reaction mixture was cooled to room temperature then N,N′-dimethylethylenediamine (0.21 mL, 1.80 mmol) and triethylamine (0.63 mL, 4.49 mmol) were added. The reaction mixture was stirred at room temperature for 3 h then concentrated in vacuo. The crude compound was diluted with ice cold water (80 mL), the resultant precipitate collected by filtration and dried in vacuo. The crude material was purified by preparative HPLC (Method A) to afford N-(3-cyano-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide (Example 66, 0.006 g, 2%) as an off white solid. ¹H NMR (400 MHz, DMSO-d₆) δ ppm 1.24-1.45 (m, 6H), 1.50-1.59 (m, 2H), 1.82-2.02 (m, 6H), 2.24 (s, 3H), 2.26-2.31 (m, 1H), 2.36-2.44 (m, 2H), 2.45 (s, 3H), 2.55-2.61 (m, 2H), 2.84-2.95 (m, 1H), 3.19-3.22 (m, 2H), 3.58-3.62 (m, 1H), 4.24-4.35 (m, 1H), 4.36-4.46 (m, 3H), 5.10-5.15 (m, 1H), 7.47-7.52 (m, 1H), 7.57-7.60 (m, 1H), 7.68-7.76 (m, 2H), 7.77-7.81 (m, 1H), 8.38-8.41 (s, 1H), 8.45 (s, 1H), 8.67 (s, 1H), 10.81 (br s, 1H), 10.99 (br s, 1H), 12.52 (br s, 1H). Mass spec m/z 764.0 [M+H]⁺.

### Example 67: N-(3-chloro-2-((R)-1-methylpyrrolidin-2-yl)-1H-pyrrolo[3,2-c]pyridin-6-yl)-1-(9-(2-(2,6-dioxopiperidin-3-yl)-7-fluoro-1-oxoisoindolin-4-yl)non-8-yn-1-yl)-3-methyl-1H-indazole-6-carboxamide

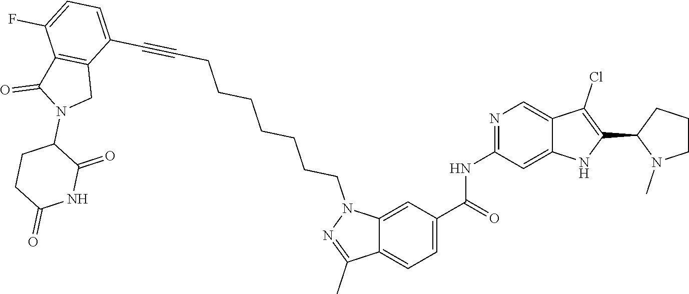 [SMILES: Cc1nn(CCCCCCCC#Cc2ccc(F)c3c2CN(C2CCC(=O)NC2=O)C3=O)c2cc(C(=O)Nc3cc4[nH]c([C@H]5CCCN5C)c(Cl)c4cn3)ccc12]

To a solution of 1-(9-(2-(2,6-dioxopiperidin-3-yl)-7-fluoro-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 52, 0.20 g, 0.3 mmol) in dimethylformamide (2.0 mL) was added N-chlorosuccinimide (0.05 g, 0.4 mmol) and the reaction mixture was stirred at room temperature for 6 h. The reaction mixture was diluted with water (80 mL) and extracted with ethyl acetate (3×100 mL). The organic layer was separated, dried over anhydrous Na₂SO₄, filtered and concentrated in vacuo. The crude material was purified by preparative HPLC (Method A) to afford N-(3-chloro-2-((R)-1-methylpyrrolidin-2-yl)-1H-pyrrolo[3,2-c]pyridin-6-yl)-1-(9-(2-(2,6-dioxopiperidin-3-yl)-7-fluoro-1-oxoisoindolin-4-yl)non-8-yn-1-yl)-3-methyl-1H-indazole-6-carboxamide (Example 67, 0.04 g, 20%) as an off white solid. ¹H NMR (400 MHz, DMSO-d₆) δ ppm 1.28-1.40 (m, 6H), 1.51-1.56 (m, 2H), 1.85-1.99 (m, 6H), 2.17 (s, 3H), 2.18-2.20 (m, 1H), 2.30-2.33 (m, 1H), 2.42 (s, 3H), 2.43-2.47 (m, 1H), 2.56-2.64 (m, 3H), 2.84-2.96 (m, 1H), 3.16-3.20 (m, 1H), 3.51-3.55 (m, 1H), 4.27-4.32 (m, 1H), 4.38-4.47 (m, 3H), 5.06-5.12 (m, 1H), 7.26 (d, J=8.80 Hz, 1H), 7.61-7.64 (m, 1H), 7.71-7.80 (m, 2H), 8.31 (s, 1H), 8.44 (s, 1H), 8.51 (s, 1H), 10.67 (s, 1H), 10.99 (s, 1H), 11.70 (s, 1H). Mass spec m/z 791.0 [M+H]⁺.

### Example 68 was Synthesized Following Scheme 65

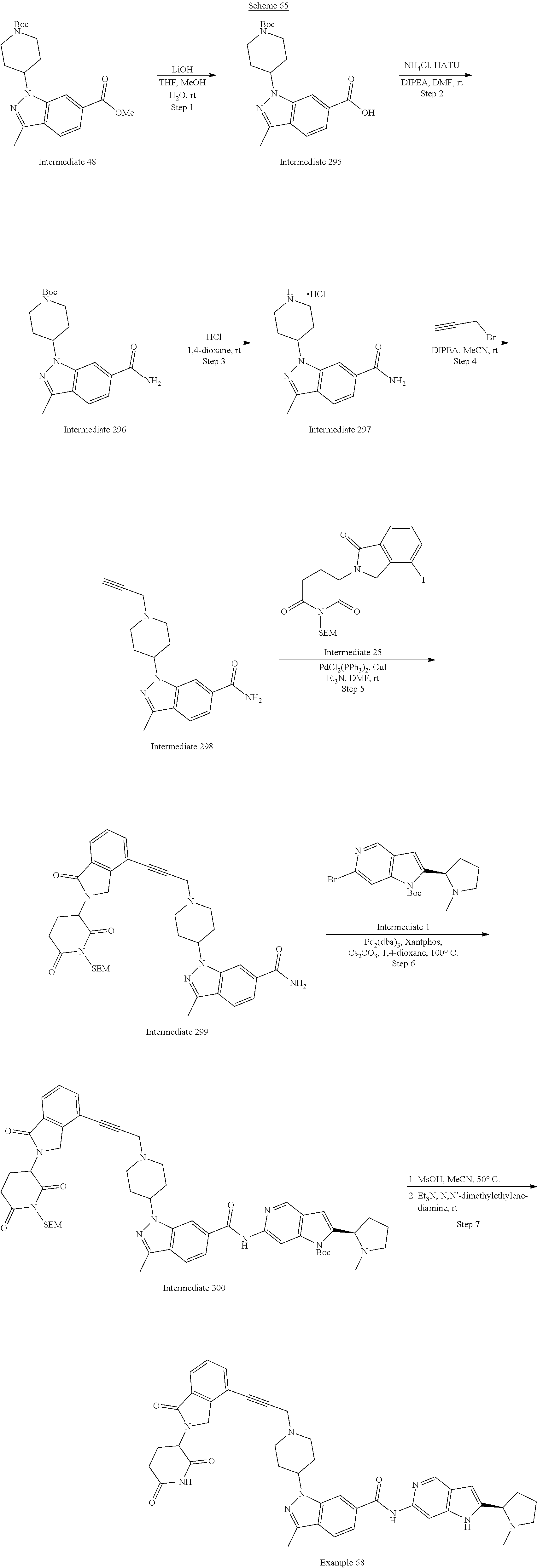

### Step 1

### Intermediate 295: 1-(1-(tert-butoxycarbonyl) piperidin-4-yl)-3-methyl-1H-indazole-6-carboxylic acid

To a solution of methyl 1-(1-(tert-butoxycarbonyl)piperidin-4-yl)-3-methyl-1H-indazole-6-carboxylate (Intermediate 48, 14.0 g, 37.49 mmol) in tetrahydrofuran (50 mL), methanol (50 mL) and water (50 mL) was added lithium hydroxide (2.70 g, 112.5 mmol). The reaction mixture was stirred at room temperature for 16 h then concentrated in vacuo. The obtained residue was diluted with water (100 mL) and extracted with dichloromethane (2×50 mL). The aqueous layer was acidified to pH~5 by addition of aqueous citric acid solution. After stirring, for 10 min, the resultant precipitate was collected by filtration and dried in vacuo to afford 1-(1-(tert-butoxycarbonyl) piperidin-4-yl)-3-methyl-1H-indazole-6-carboxylic acid (Intermediate 295, 12.5 g, 93%) as an off-white solid which was used for the next step without further purification. ¹H NMR (400 MHz, DMSO-d₆) δ ppm 1.43 (s, 9H), 1.86-1.97 (m, 4H), 2.52 (s, 3H), 2.98-3.04 (m, 2H), 4.06-4.10 (m, 2H), 4.84-4.93 (m, 1H), 7.65-7.69 (m, 1H), 7.70-7.74 (m, 1H), 8.26 (s, 1H). Mass spec m/z 360.30 [M+H]⁺.

### Step 2

### Intermediate 296: tert-butyl 4-(6-carbamoyl-3-methyl-1H-indazol-1-yl) piperidine-1-carboxylate

To a solution of 1-(1-(tert-butoxycarbonyl) piperidin-4-yl)-3-methyl-1H-indazole-6-carboxylic acid (Intermediate 295, 12.5 g, 34.8 mmol) in dimethylformamide (130 mL) was added HATU (26.4 g, 69.6 mmol) and the reaction mixture stirred at room temperature for 15 min. Ammonium chloride (9.30 g, 174.0 mmol) and DIPEA (20 mL, 104.0 mmol) were then added and the reaction mixture was stirred at room temperature for 16 h. The reaction mixture was then poured into ice cold water (250 mL), the resultant precipitate was collected by filtration and dried in vacuo to afford tert-butyl 4-(6-carbamoyl-3-methyl-1H-indazol-1-yl) piperidine-1-carboxylate (Intermediate 296, 11.0 g, 88%) as an off white solid, which was used for next step without further purification. ¹H NMR (400 MHz, DMSO-d₆) δ ppm 1.43 (s, 9H), 1.90-1.98 (m, 4H), 2.53 (s, 3H), 2.97-3.14 (m, 2H), 4.04-4.14 (m, 2H), 4.73-4.86 (m, 1H), 7.43 (br s, 1H), 7.59 (d, J=8.80 Hz, 1H), 7.73 (d, J=8.80 Hz, 1H), 8.04 (br s, 1H), 8.19 (s, 1H). Mass spec m/z 359.20 [M+H]⁺.

### Step 3

### Intermediate 297: 3-methyl-1-(piperidin-4-yl)-1H-indazole-6-carboxamide hydrochloride

To a solution of tert-butyl 4-(6-carbamoyl-3-methyl-1H-indazol-1-yl) piperidine-1-carboxylate (Intermediate 296, 9.00 g, 25.1 mmol) in 1,4-dioxane (90 mL) was added 4M HCl in 1,4-dioxane (250 mL). The reaction mixture was stirred at room temperature for 16 h. The reaction mixture was concentrated in vacuo and the obtained solid washed with diethyl ether (2×100 mL) and dried in vacuo to afford 3-methyl-1-(piperidin-4-yl)-1H-indazole-6-carboxamide hydrochloride (Intermediate 297, 9.00 g, 85%) as an off-white solid, which was used for the next step without further purification. ¹H NMR (400 MHz, DMSO-d₆) δ ppm 2.01-2.10 (m, 2H), 2.29-2.41 (m, 2H), 2.50 (s, 3H), 3.05-3.18 (m, 2H), 3.38-3.44 (m, 2H), 4.92-4.98 (m, 1H), 7.44 (br s, 1H), 7.61 (d, J=8.20 Hz, 1H), 7.73 (d, J=8.20 Hz, 1H), 8.09 (br s, 1H), 8.31 (s, 1H), 9.08 (br s, 1H), 9.27 (br s, 1H). Mass spec m/z 259.30 [M+H]⁺.

### Step 4

### Intermediate 298: 3-methyl-1-(1-(prop-2-yn-1-yl) piperidin-4-yl)-1H-indazole-6-carboxamide

To a cooled (0° C.) solution of 3-methyl-1-(piperidin-4-yl)-1H-indazole-6-carboxamide hydrochloride (Intermediate 297, 1.00 g, 3.0 mmol) in acetonitrile (10 mL) was added DIPEA (1.00 g, 10.0 mmol) followed by 3-bromoprop-1-yne (CAS No: 106-96-7, 0.40 g, 3.0 mmol). The reaction mixture was stirred at room temperature for 1 h then poured into ice cold water (80 mL) and extracted with ethyl acetate (3×100 mL). The combined organic layers were dried over anhydrous Na₂SO₄, filtered and concentrated in vacuo. The crude material was purified by combi-flash column chromatography, by eluting with 7% of MeOH in DCM, to afford 3-methyl-1-(1-(prop-2-yn-1-yl) piperidin-4-yl)-1H-indazole-6-carboxamide (Intermediate 298, 0.50 g, 30%) as an off white solid. ¹H NMR (400 MHz, DMSO-d₆) δ ppm 1.88-1.94 (m, 2H), 2.09-2.18 (m, 2H), 2.36-2.46 (m, 2H), 2.49 (s, 3H), 2.92-2.96 (m, 2H), 3.15 (s, 1H), 3.37 (s, 2H), 4.46-4.58 (m, 1H), 7.41 (br s, 1H), 7.60 (d, J=8.40 Hz, 1H), 7.72 (d, J=8.40 Hz, 1H), 8.03 (br s, 1H), 8.20 (s, 1H). Mass spec m/z 297.18 [M+H]⁺.

### Step 5

### Intermediate 299: 1-(1-(3-(2-(2,6-dioxo-1-((2-(trimethylsilyl)ethoxy)methyl)piperidin-3-yl)-1-oxoisoindolin-4-yl) prop-2-yn-1-yl) piperidin-4-yl)-3-methyl-1H-indazole-6-carboxamide

To a solution of 3-methyl-1-(1-(prop-2-yn-1-yl) piperidin-4-yl)-1H-indazole-6-carboxamide (Intermediate 298, 0.5 g, 1.68 mmol) in dimethylformamide (1 mL) was added 3-(4-iodo-1-oxoisoindolin-2-yl)-1-((2-(trimethylsilyl)ethoxy)methyl)piperidine-2,6-dione (Intermediate 25, 0.84 g, 1.68 mmol) followed by triethylamine (8.74 mL, 62.42 mmol). The reaction mixture was purged with argon for 15 min, then copper (I) iodide (0.05 g, 0.25 mmol) and PdCl₂(PPh₃)₂ (0.18 g, 0.25 mmol) were added. The reaction mixture was purged with argon for another 10 min and then stirred at room temperature for 1 h. The reaction mixture was then quenched with ice cold water (80 mL) and extracted with ethyl acetate (3×50 mL). The combined organic layers were dried over anhydrous Na₂SO₄ and concentrated in vacuo. The crude material was purified by combi-flash column chromatography, by eluting with 7% MeOH in DCM, to afford 1-(1-(3-(2-(2,6-dioxo-1-((2-(trimethylsilyl) ethoxy) methyl) piperidin-3-yl)-1-oxoisoindolin-4-yl) prop-2-yn-1-yl) piperidin-4-yl)-3-methyl-1H-indazole-6-carboxamide (Intermediate 299, 0.38 g, 34%) as a yellow solid. ¹H NMR (400 MHz, DMSO-d₆) δ ppm −0.05 (s, 9H), 0.75-0.88 (m, 2H), 1.92-1.96 (m, 2H), 2.06-2.23 (m, 3H), 2.44-2.47 (m, 2H), 2.53 (s, 3H), 2.76-2.80 (m, 2H), 2.98-3.09 (m, 3H), 3.45-3.57 (m, 2H), 3.69 (s, 2H), 4.31-4.42 (m, 1H), 4.52-4.64 (m, 2H), 5.00-5.10 (m, 2H), 5.25-5.30 (m, 1H), 7.41 (br s, 1H), 7.55-7.63 (m, 2H), 7.68-7.81 (m, 3H), 8.00 (br s, 1H), 8.20 (s, 1H). Mass spec m/z 669.10 [M+H]⁺.

### Step 6

### Intermediate 300: tert-butyl 6-(1-(1-(3-(2-(2,6-dioxo-1-((2-(trimethylsilyl)ethoxy)methyl)piperidin-3-yl)-1-oxoisoindolin-4-yl)prop-2-yn-1-yl)piperidin-4-yl)-3-methyl-1H-indazole-6-carboxamido)-2-((R)-1-methylpyrrolidin-2-yl)-1H-pyrrolo[3,2-c]pyridine-1-carboxylate

To a solution of 1-(1-(3-(2-(2,6-dioxo-1-((2-(trimethylsilyl) ethoxy) methyl) piperidin-3-yl)-1-oxoisoindolin-4-yl) prop-2-yn-1-yl) piperidin-4-yl)-3-methyl-1H-indazole-6-carboxamide (Intermediate 299, 0.34 g, 0.50 mmol) in 1,4-dioxane (3 mL) was added tert-butyl (R)-6-bromo-2-(1-methylpyrrolidin-2-yl)-1H-pyrrolo[3,2-c]pyridine-1-carboxylate (Intermediate 1, 0.23 g, 0.61 mmol) followed by cesium carbonate (0.34 g, 1.01 mmol). The reaction mixture was purged with argon for 15 min, then Pd₂(dba)₃ (0.06 g, 0.07 mmol) and Xantphos (0.09 g, 0.15 mmol) were added. The reaction mixture was further purged with argon for 10 min and stirred at 100° C. for 2 h. The reaction mixture was concentrated in vacuo and the crude material was purified by combi-flash chromatography, by eluting with 7% MeOH in DCM, to afford tert-butyl 6-(1-(1-(3-(2-(2,6-dioxo-1-((2-(trimethylsilyl)ethoxy)methyl)piperidin-3-yl)-1-oxoisoindolin-4-yl)prop-2-yn-1-yl)piperidin-4-yl)-3-methyl-1H-indazole-6-carboxamido)-2-((R)-1-methylpyrrolidin-2-yl)-1H-pyrrolo[3,2-c]pyridine-1-carboxylate (Intermediate 300, 0.25 g, 31%) as yellow gummy solid. ¹H NMR (400 MHz, DMSO-d₆) δ ppm −0.07 (s, 9H), 0.75-0.82 (m, 2H), 1.64-1.68 (m, 2H), 1.70 (s, 9H), 1.73-1.82 (m, 3H), 2.06-2.28 (m, 4H), 2.35 (s, 3H), 2.38-2.44 (m, 2H), 2.52 (s, 3H), 2.55-2.64 (m, 2H), 2.97-3.08 (m, 3H), 3.09-3.17 (m, 2H), 3.45-3.53 (m, 2H), 3.71 (s, 2H), 3.87-3.96 (m, 1H), 4.34-4.38 (m, 1H), 4.54-4.70 (m, 2H), 4.96-5.07 (m, 2H), 5.21-5.30 (m, 1H), 6.75 (s, 1H), 7.56-7.60 (m, 1H), 7.68-7.74 (m, 1H), 7.75-7.82 (m, 3H), 8.48 (s, 1H), 8.59 (s, 1H), 8.97 (s, 1H), 10.72 (s, 1H). Mass spec m/z 968.80 [M+H]⁺.

### Step 7

### Example 68: 1-(1-(3-(2-(2,6-dioxopiperidin-3-yl)-1-oxoisoindolin-4-yl)prop-2-yn-1-yl)piperidin-4-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(1-(3-(2-(2,6-dioxo-1-((2-(trimethylsilyl)ethoxy)methyl)piperidin-3-yl)-1-oxoisoindolin-4-yl)prop-2-yn-1-yl)piperidin-4-yl)-3-methyl-1H-indazole-6-carboxamido)-2-((R)-1-methylpyrrolidin-2-yl)-1H-pyrrolo[3,2-c]pyridine-1-carboxylate (Intermediate 300, 0.23 g, 0.23 mmol) in acetonitrile (3 mL) was added methanesulfonic acid (0.24 g, 2.37 mmol) and the reaction mixture was stirred at 50° C. for 2 h. The reaction mixture was cooled to room temperature, then N,N′-dimethylethylenediamine (0.13 g, 1.42 mmol) followed by triethylamine (0.50 g, 4.75 mmol) were added and the mixture stirred at room temperature for 2 h. The reaction mixture was poured into ice cold water (30 mL), the resultant precipitate was collected by filtration and dried in vacuo. The crude material was purified by preparative HPLC (Method A) to afford 1-(1-(3-(2-(2,6-dioxopiperidin-3-yl)-1-oxoisoindolin-4-yl)prop-2-yn-1-yl)piperidin-4-yl)-3-methyl-N-(2-((R)-1-methylpyrrolidin-2-yl)-1H-pyrrolo[3,2-c]pyridin-6-yl)-1H-indazole-6-carboxamide (Example 68, 0.04 g, 19%) as white solid. ¹H NMR (400 MHz, DMSO-d₆) δ ppm 1.76-1.95 (m, 3H), 1.97-2.05 (m, 2H), 2.06-2.14 (m, 2H), 2.17 (s, 3H), 2.20-2.30 (m, 2H), 2.40-2.46 (m, 2H), 2.53 (s, 3H), 2.54-2.65 (m, 3H), 2.81-2.91 (m, 1H), 3.01-3.08 (m, 2H), 3.11-3.18 (m, 1H), 3.32-3.35 (m, 1H), 3.72 (s, 2H), 4.37-4.43 (m, 1H), 4.52-4.58 (m, 1H), 4.64-4.73 (m, 1H), 5.11-5.16 (m, 1H), 6.40 (s, 1H), 7.55-7.60 (m, 1H), 7.71-7.81 (m, 4H), 8.25 (s, 1H), 8.49 (s, 1H), 8.50 (s, 1H), 10.49 (s, 1H), 10.98 (s, 1H), 11.38 (s, 1H). Mass spec m/z 738.20 [M+H]⁺.

### Example 69 was Synthesized Following Scheme 66

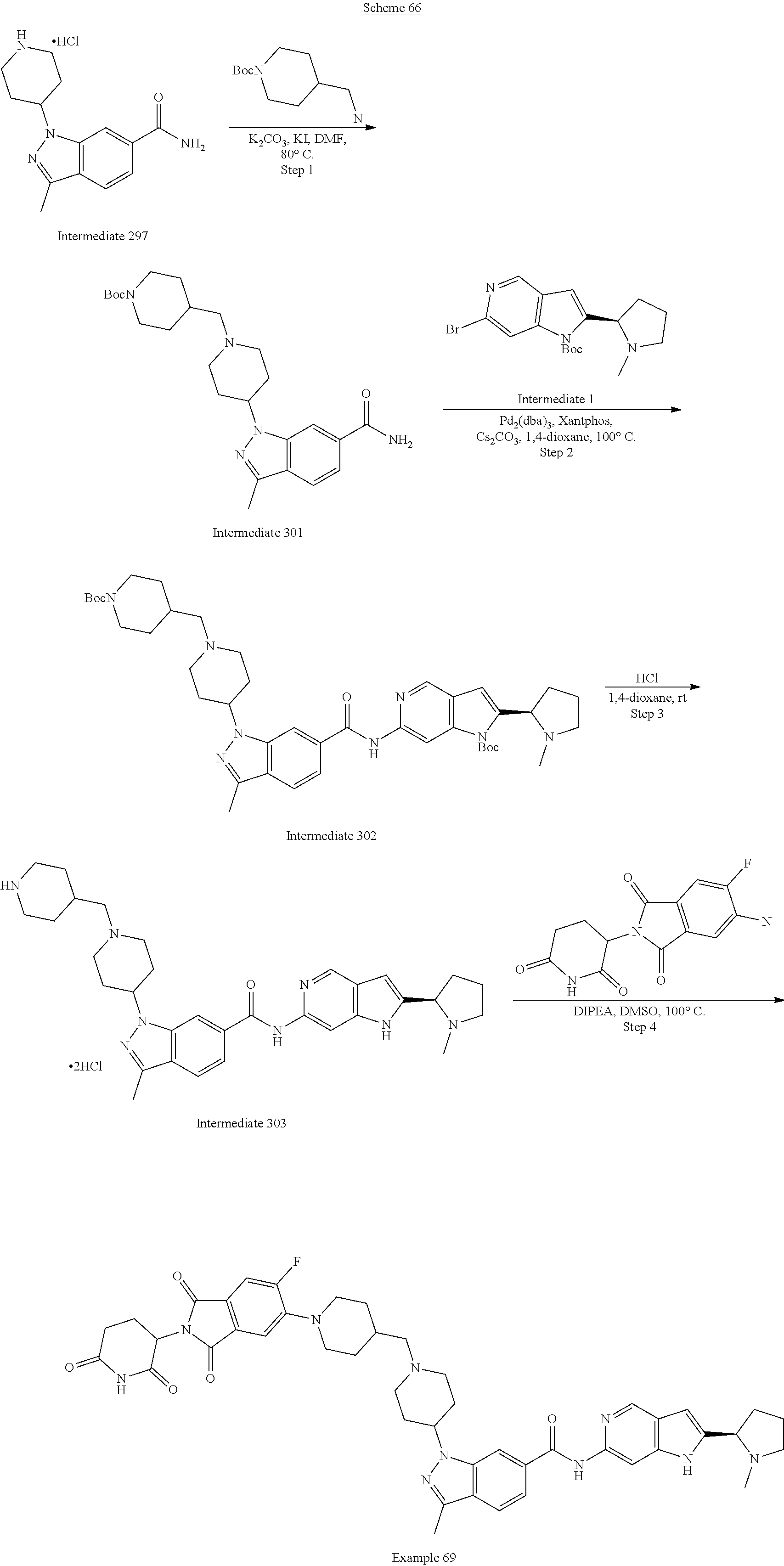

### Step 1

### Intermediate 301: tert-butyl 4-((4-(6-carbamoyl-3-methyl-1H-indazol-1-yl) piperidin-1-yl) methyl) piperidine-1-carboxylate

To a solution of 3-methyl-1-(4-piperidyl) indazole-6-carboxamide hydrochloride (Intermediate 297, 5.00 g, 17.0 mmol) in dimethylformamide (50 mL) was added tert-butyl 4-(bromomethyl)piperidine-1-carboxylate (CAS No: 158407-04-6, 5.66 g, 20.4 mmol) followed by potassium carbonate (7.03 g, 50.9 mmol) and potassium iodide (0.28 g, 1.70 mmol). The reaction mixture was stirred at 80° C. for 16 h then concentrated in vacuo. The obtained residue was poured into ice cold water (800 mL), the resultant precipitate was collected by filtration and dried in vacuo to afford tert-butyl 4-((4-(6-carbamoyl-3-methyl-1H-indazol-1-yl)piperidin-1-yl)methyl)piperidine-1-carboxylate (Intermediate 297, 3.90 g, 50%) as an off white solid which was used for the next step without further purification. ¹H NMR (400 MHz, DMSO-d₆) δ ppm 1.39 (s, 9H), 1.62-1.76 (m, 4H), 1.82-1.90 (m, 2H), 2.06-2.20 (m, 5H), 2.52 (s, 3H), 2.60-2.80 (m, 3H), 2.87-3.05 (m, 2H), 3.85-4.05 (m, 3H), 4.46-4.60 (m, 1H), 7.41 (br s, 1H), 7.58-7.62 (m, 1H), 7.70-7.74 (m, 1H), 8.03 (br s, 1H), 8.18 (s, 1H). Mass spec m/z 456.3 [M+H]⁺.

### Step 2

### Intermediate 302: tert-butyl (R)-6-(1-(1-((1-(tert-butoxycarbonyl) piperidin-4-yl) methyl) piperidin-4-yl)-3-methyl-1H-indazole-6-carboxamido)-2-(1-methylpyrrolidin-2-yl)-1H-pyrrolo [3,2-c]pyridine-1-carboxylate

To a solution of tert-butyl 4-((4-(6-carbamoyl-3-methyl-1H-indazol-1-yl)piperidin-1-yl)methyl)piperidine-1-carboxylate (Intermediate 301, 1.92 g, 4.21 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 1, 2.00 g, 5.26 mmol) followed by cesium carbonate (4.06 g, 21.0 mmol). The reaction mixture was purged with argon for 15 min, then Pd₂(dba)₃ (0.74 g, 0.78 mmol) and Xantphos (0.94 g, 1.58 mmol) were added. The reaction mixture was purged with argon for a further 10 min and then stirred at 100° C. for 2 h. The reaction mixture was filtered through celite, the filter pad was washed with ethyl acetate (100 mL) and the filtrate was concentrated in vacuo. The crude material was purified by combi-flash chromatography, by eluting with 10% MeOH in DCM, to afford tert-butyl (R)-6-(1-(1-((1-(tert-butoxycarbonyl)piperidin-4-yl)methyl)piperidin-4-yl)-3-methyl-1H-indazole-6-carboxamido)-2-(1-methylpyrrolidin-2-yl)-1H-pyrrolo[3,2-c]pyridine-1-carboxylate (Intermediate 302, 1.60 g, 40%) as a yellow solid. ¹H NMR (400 MHz, DMSO-d₆) δ ppm 0.89-1.03 (m, 2H), 1.07-1.10 (m, 1H), 1.39 (s, 9H), 1.55-1.66 (m, 3H), 1.70 (s, 9H), 1.75-1.80 (m, 3H), 1.88-1.94 (m, 2H), 2.07-2.24 (m, 6H), 2.28-2.33 (m, 1H), 2.35 (s, 3H), 2.39-2.44 (m, 1H), 2.52 (s, 3H), 2.92-3.02 (m, 2H), 3.08-3.16 (m, 1H), 3.36-3.42 (m, 1H), 3.85-4.00 (m, 3H), 4.60-4.70 (m, 1H), 6.77 (s, 1H), 7.71 (d, J=8.40 Hz, 1H), 7.77 (d, J=8.40 Hz, 1H), 8.45 (s, 1H), 8.61 (s, 1H), 8.99 (s, 1H), 10.78 (br s, 1H). Mass spec m/z 755.5 [M+H]⁺.

### Step 3

### Intermediate 303: (R)-3-methyl-N-(2-(1-methylpyrrolidin-2-yl)-1H-pyrrolo[3,2-c]pyridin-6-yl)-1-(1-(piperidin-4-ylmethyl)piperidin-4-yl)-1H-indazole-6-carboxamide dihydrochloride

To a cooled (0° C.) solution of tert-butyl (R)-6-(1-(1-((1-(tert-butoxycarbonyl)piperidin-4-yl)methyl)piperidin-4-yl)-3-methyl-1H-indazole-6-carboxamido)-2-(1-methylpyrrolidin-2-yl)-1H-pyrrolo[3,2-c]pyridine-1-carboxylate (Intermediate 302, 1.50 g, 1.99 mmol) in 1,4-dioxane (18 mL) was added 4M HCl in 1,4-dioxane (20 mL, 80.0 mmol). The reaction mixture was stirred at room temperature for 16 h then concentrated in vacuo to afford (R)-3-methyl-N-(2-(1-methylpyrrolidin-2-yl)-1H-pyrrolo[3,2-c]pyridin-6-yl)-1-(1-(piperidin-4-ylmethyl)piperidin-4-yl)-1H-indazole-6-carboxamide dihydrochloride (Intermediate 303, 1.10 g) as a yellow solid which was used for the next step without further purification. ¹H NMR (400 MHz, DMSO-d₆) δ ppm 1.41-1.57 (m, 2H) 2.01-2.10 (m, 2H), 2.14-2.21 (m, 2H), 2.26 (s, 3H), 2.33-2.44 (m, 2H), 2.56 (s, 3H), 2.65-2.77 (m, 3H), 2.84-2.92 (m, 3H), 3.05-3.12 (m, 2H), 3.17-3.33 (m, 5H), 3.72-3.76 (m, 3H), 4.75-4.80 (m, 1H), 5.01-5.08 (m, 2H), 7.32 (s, 1H), 7.83-7.88 (m, 1H), 7.90-7.96 (m, 1H), 8.44-8.48 (m, 1H), 9.00 (s, 1H), 9.14 (s, 1H), 10.59 (br s, 1H), 11.65 (br s, 1H), 12.40 (br s, 1H), 13.43 (br s, 1H). Mass spec m/z 555.3 [M+H]⁺.

### Step 4

### Example 69: 1-(1-((1-(2-(2,6-dioxopiperidin-3-yl)-6-fluoro-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide

To a solution of (R)-3-methyl-N-(2-(1-methylpyrrolidin-2-yl)-1H-pyrrolo [3,2-c]pyridin-6-yl)-1-(1-(piperidin-4-ylmethyl) piperidin-4-yl)-1H-indazole-6-carboxamide dihydrochloride (Intermediate 303, 0.30 g, 0.51 mmol) in dimethyl sulfoxide (8 mL) was added 2-(2,6-dioxopiperidin-3-yl)-5,6-difluoroisoindoline-1,3-dione (CAS No: 1496997-41-1, 0.15 g, 0.51 mmol) followed by DIPEA (0.44 mL, 2.5 mmol). The reaction mixture was stirred at 100° C. for 2 h then concentrated in vacuo. The obtained residue was poured into ice cold water (80 mL), the resultant precipitate collected by filtration and dried in vacuo to afford the crude compound. This material was purified by preparative HPLC (Method A) to afford 1-(1-((1-(2-(2,6-dioxopiperidin-3-yl)-6-fluoro-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide (Example 69, 0.012 g, 3%) as a yellow solid. ¹H NMR (400 MHz, DMSO-d₆) δ ppm 1.24-1.36 (m, 2H), 1.73-1.98 (m, 8H), 2.00-2.07 (m, 1H), 2.10-2.16 (m, 2H), 2.18 (s, 3H), 2.20-2.25 (m, 3H), 2.26-2.31 (m, 3H), 2.46-2.48 (m, 1H), 2.53 (s, 3H), 2.55-2.63 (m, 2H), 2.86-2.97 (m, 3H), 2.99-3.07 (m, 2H), 3.11-3.19 (m, 1H), 3.58-3.68 (m, 2H), 4.62-4.72 (m, 1H), 5.08-5.12 (m, 1H), 6.41 (s, 1H), 7.44-7.46 (m, 1H), 7.69-7.80 (m, 3H), 8.25 (s, 1H), 8.46 (s, 1H), 8.53 (s, 1H), 10.56 (br s, 1H), 11.10 (br s, 1H), 11.40 (br s, 1H). Mass spec m/z 829.1 [M+H]⁺.

### Example 70: 1-(1-((1-(2-(2,6-dioxopiperidin-3-yl)-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide

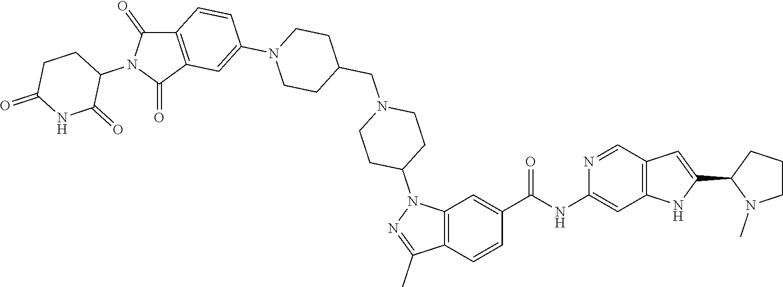 [SMILES: Cc1nn(C2CCN(CC3CCN(c4ccc5c(c4)C(=O)N(C4CCC(=O)NC4=O)C5=O)CC3)CC2)c2cc(C(=O)Nc3cc4[nH]c([C@H]5CCCN5C)cc4cn3)ccc12]

To a solution of (R)-3-methyl-N-(2-(1-methylpyrrolidin-2-yl)-1H-pyrrolo[3,2-c]pyridin-6-yl)-1-(1-(piperidin-4-ylmethyl)piperidin-4-yl)-1H-indazole-6-carboxamide dihydrochloride (Intermediate 303, 0.35 g, 0.63 mmol) in dimethyl sulfoxide (8.0 mL) was added 2-(2,6-dioxopiperidin-3-yl)-5-fluoroisoindoline-1,3-dione (CAS No: 835616-61-0, 0.14 g, 0.50 mmol) followed by DIPEA (3.31 mL, 18.93 mmol) and the reaction mixture was heated under microwave irradiation at 100° C. for 2 h. The reaction mixture was poured into ice cold water (50 mL), the resultant precipitate was collected by filtration, washed with pentane (2×40 mL) and dried in vacuo. The crude material was purified by preparative HPLC (Method A) to afford 1-(1-((1-(2-(2,6-dioxopiperidin-3-yl)-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide (Example 70, 0.045 g, 9%) as a yellow solid. ¹H NMR (400 MHz, DMSO-d₆) δ ppm 1.11-1.25 (m, 2H), 1.76-2.06 (m, 9H), 2.12-2.15 (m, 2H), 2.17 (s, 3H), 2.18-2.22 (m, 2H), 2.23-2.29 (m, 3H), 2.53 (s, 3H), 2.54-2.63 (m, 3H), 2.84-3.07 (m, 6H), 3.11-3.18 (m, 1H), 4.04-4.12 (m, 2H), 4.62-4.70 (m, 1H), 5.05-5.09 (m, 1H), 6.41 (s, 1H), 7.22-7.26 (m, 1H), 7.32 (s, 1H), 7.64-7.68 (m, 1H), 7.71-7.74 (m, 1H), 7.76-7.80 (m, 1H), 8.25 (s, 1H), 8.46 (s, 1H), 8.53 (s, 1H), 10.57 (br s, 1H), 11.09 (br s, 1H), 11.41 (br s, 1H). Mass spec m/z 811.1 [M+H]⁺.

### Example 71: 1-(1-((1-(2-(2,6-dioxopiperidin-3-yl)-1,3-dioxoisoindolin-4-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide

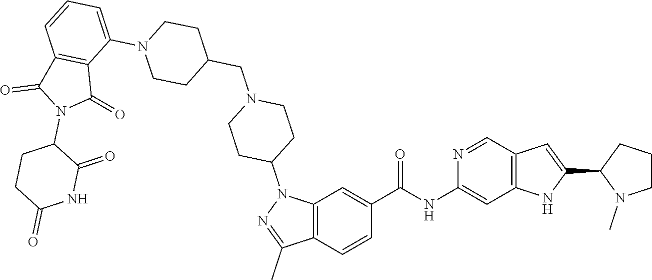 [SMILES: Cc1nn(C2CCN(CC3CCN(c4cccc5c4C(=O)N(C4CCC(=O)NC4=O)C5=O)CC3)CC2)c2cc(C(=O)Nc3cc4[nH]c([C@H]5CCCN5C)cc4cn3)ccc12]

To a solution of (R)-3-methyl-N-(2-(1-methylpyrrolidin-2-yl)-1H-pyrrolo[3,2-c]pyridin-6-yl)-1-(1-(piperidin-4-ylmethyl)piperidin-4-yl)-1H-indazole-6-carboxamide dihydrochloride (Intermediate 303, 0.40 g, 0.68 mmol) in dimethyl sulfoxide (8.0 mL) was added 2-(2,6-dioxopiperidin-3-yl)-4-fluoroisoindoline-1,3-dione (CAS No: 835616-60-9, 0.19 g, 0.68 mmol) followed by DIPEA (0.59 mL, 3.4 mmol) and the reaction mixture was stirred at 100° C. for 4h. The reaction mixture was poured into ice cold water (80 mL), the resultant precipitate was collected by filtration and dried in vacuo. The crude material was purified by preparative HPLC (Method A) to afford 1-(1-((1-(2-(2,6-dioxopiperidin-3-yl)-1,3-dioxoisoindolin-4-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide (Example 71, 0.18 g, 34%) as a yellow solid. ¹H NMR (400 MHz, DMSO-d₆) δ ppm 1.30-1.43 (m, 2H), 1.72-1.83 (m, 2H), 1.84-1.97 (m, 6H), 2.00-2.06 (m, 1H), 2.10-2.16 (m, 2H), 2.18 (s, 3H), 2.19-2.32 (m, 6H), 2.53 (s, 3H), 2.56-2.63 (m, 2H), 2.86-2.95 (m, 3H), 3.02-3.08 (m, 2H), 3.10-3.20 (m, 1H), 3.32-3.37 (m, 1H), 3.70-3.74 (m, 2H), 4.66-4.70 (m, 1H), 5.07-5.12 (m, 1H), 6.41 (s, 1H), 7.31-7.36 (m, 2H), 7.66-7.74 (m, 2H), 7.76-7.80 (m, 1H), 8.25 (s, 1H), 8.47 (s, 1H), 8.53 (s, 1H), 10.55 (br s, 1H), 11.08 (br s, 1H), 11.39 (br s, 1H). Mass spec m/z 811.1 [M+H]⁺.

### Example 72 was Synthesized Following Scheme 67

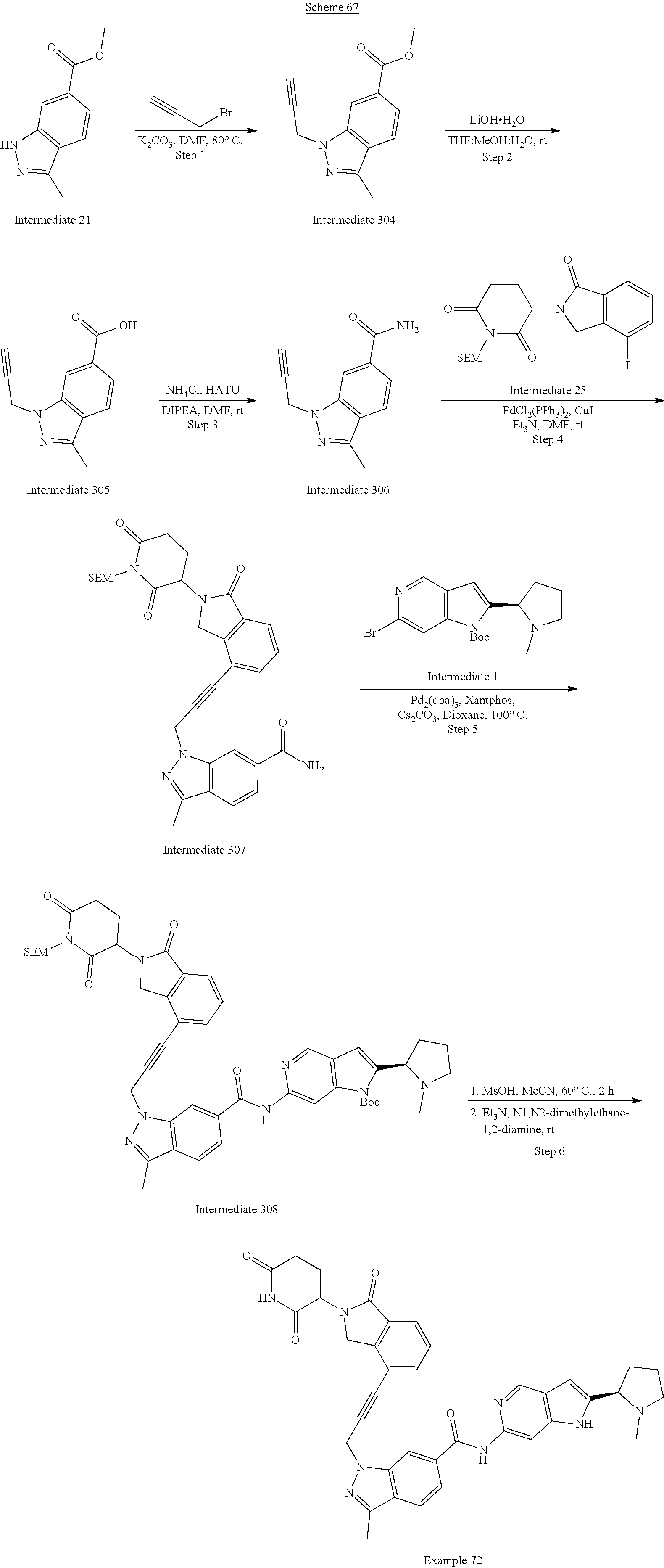

### Step 1

### Intermediate 304: methyl 3-methyl-1-(prop-2-yn-1-yl)-1H-indazole-6-carboxylate

To a solution of methyl 3-methyl-1H-indazole-6-carboxylate (Intermediate 21, 5.0 g, 28.0 mmol) in dimethylformamide (50 mL) was added 3-bromoprop-1-yne (CAS No: 106-96-7, 4.4 g, 37.0 mmol) followed by potassium carbonate (12.0 g, 85.0 mmol) and the reaction mixture was stirred at 80° C. for 2 h. The reaction mixture was quenched with water (200 mL) and extracted with ethyl acetate (2×200 mL). The combined organic layers were separated, dried over anhydrous Na₂SO₄, filtered, and concentrated in vacuo to get the crude mixture of regioisomers. The regioisomers were separated by combi-flash chromatography, by eluting with 10% ethyl acetate in heptane, to afford the desired regioisomer methyl 3-methyl-1-(prop-2-yn-1-yl)-1H-indazole-6-carboxylate (Intermediate 304, 3.70 g, 57%) as a pale yellow solid. ¹H NMR (400 MHz, DMSO-d₆) δ ppm 2.52 (s, 3H), 3.37 (s, 1H), 3.91 (s, 3H), 5.37 (s, 2H), 7.71 (d, J=8.29 Hz, 1H), 7.86 (d, J=8.29 Hz, 1H), 8.33 (s, 1H). Mass spec m/z 229.4 [M+H]⁺.

### Step 2

### Intermediate 305: 3-methyl-1-(prop-2-yn-1-yl)-1H-indazole-6-carboxylic acid

To a solution of methyl 3-methyl-1-(prop-2-yn-1-yl)-1H-indazole-6-carboxylate (Intermediate 304, 3.70 g, 16.0 mmol) in tetrahydrofuran (20 mL), methanol (20 mL) and water (10 mL) was added lithium hydroxide (1.9 g, 81.0 mmol). The reaction mixture was stirred at room temperature for 2 h then concentrated in vacuo. The obtained residue was poured in water (30 mL) and acidified to pH~4 with saturated solution of citric acid. The resultant precipitate was collected by filtration, washed with water (50 mL) and dried in vacuo to afford 3-methyl-1-(prop-2-yn-1-yl)-1H-indazole-6-carboxylic acid (Intermediate 305, 3.55 g) as an off white solid, which was used for the next step without further purification. ¹H NMR (400 MHz, DMSO-d₆) δ ppm 2.54 (s, 3H), 3.36 (s, 1H), 5.34 (s, 2H), 7.69-7.71 (m, 1H), 7.80-7.84 (m, 1H), 8.29 (s, 1H). Mass spec m/z 215.1 [M+H]⁺.

### Step 3

### Intermediate 306: 3-methyl-1-(prop-2-yn-1-yl)-1H-indazole-6-carboxamide

To a solution of 3-methyl-1-(prop-2-yn-1-yl)-1H-indazole-6-carboxylic acid (Intermediate 305, 3.55 g, 16.6 mmol) in dimethylformamide (35 mL) was added HATU (9.55 g, 24.9 mmol) and the reaction mixture was stirred for 15 min. Ammonium chloride (4.43 g, 82.9 mmol) and DIPEA (11.7 mL, 66.3 mmol) were then added and the reaction mixture was stirred at room temperature for 16 h. The reaction mixture was quenched with ice cold water (200 mL) and extracted with ethyl acetate (2×200 mL). The combined organic layers were separated, dried over anhydrous Na₂SO₄, filtered, and concentrated in vacuo. The crude material was purified by preparative HPLC (Method A) to afford 3-methyl-1-(prop-2-yn-1-yl)-1H-indazole-6-carboxamide (Intermediate 306, 1.20 g, 34%) as an off white solid. ¹H NMR (400 MHz, DMSO-d₆) δ ppm 2.49 (s, 3H), 3.35 (s, 1H), 5.27 (s, 2H), 7.45 (br s, 1H), 7.65 (d, J=8.20 Hz, 1H), 7.78 (d, J=8.20 Hz, 1H), 8.04 (br s, 1H), 8.20 (s, 1H). Mass spec m/z 214.4 [M+H]⁺.

### Step 4

### Intermediate 307: 1-(3-(2-(2,6-dioxo-1-((2-(trimethylsilyl)ethoxy)methyl) piperidin-3-yl)-1-oxoisoindolin-4-yl) prop-2-yn-1-yl)-3-methyl-1H-indazole-6-carboxamide

To a solution of 3-methyl-1-(prop-2-yn-1-yl)-1H-indazole-6-carboxamide (Intermediate 306, 0.60 g, 2.81 mmol) in dimethylformamide (5.0 mL) was added 3-(4-iodo-1-oxoisoindolin-2-yl)-1-((2-(trimethylsilyl)ethoxy)methyl)piperidine-2,6-dione (Intermediate 25, 1.40 g, 2.81 mmol) followed by triethylamine (14.6 mL, 104.11 mmol). The reaction mixture was purged with argon for 15 min, then copper (I) iodide (0.084 g, 0.42 mmol) and PdCl₂(PPh₃)₂ (0.20 g, 0.28 mmol) were added. The reaction mixture was purged with argon for another 10 min and stirred at room temperature for 2 h. The reaction mixture was diluted with water (100 mL) and extracted with ethyl acetate (3×100 mL). The combined organic layers were separated, dried over Na₂SO₄, and concentrated in vacuo. The crude material was purified by combi-flash chromatography, by eluting with 60% ethyl acetate in heptane, to afford 1-(3-(2-(2,6-dioxo-1-((2-(trimethylsilyl)ethoxy)methyl)piperidin-3-yl)-1-oxoisoindolin-4-yl) prop-2-yn-1-yl)-3-methyl-1H-indazole-6-carboxamide (Intermediate 307, 0.80 g, 49%) as a white solid. ¹H NMR (400 MHz, DMSO-d₆) δ ppm −0.04 (s, 9H), 0.78-0.89 (m, 2H), 1.99-2.06 (m, 1H), 2.26-2.38 (m, 1H), 2.52 (s, 3H), 2.79-2.84 (m, 1H), 3.01-3.12 (m, 1H), 3.50-3.56 (m, 2H), 4.12-4.19 (m, 1H), 4.32-4.36 (m, 1H), 5.06 (s, 2H), 5.21-5.26 (m, 1H), 5.59 (s, 2H), 7.39 (br s, 1H), 7.51-7.56 (m, 1H), 7.66-7.68 (m, 2H), 7.74-7.76 (m, 1H), 7.78-7.81 (m, 1H), 8.08 (br s, 1H), 8.32 (s, 1H). Mass spec m/z 584.1 [M+H]⁺.

### Step 5

### Intermediate 308: tert-butyl-6-(1-(3-(2-(2,6-dioxo-1-((2-(trimethylsilyl)ethoxy)methyl)piperidin-3-yl)-1-oxoisoindolin-4-yl)prop-2-yn-1-yl)-3-methyl-1H-indazole-6-carboxamido)-2-((R)-1-methylpyrrolidin-2-yl)-1H-pyrrolo[3,2-c]pyridine-1-carboxylate

To a solution of 1-(3-(2-(2,6-dioxo-1-((2-(trimethylsilyl) ethoxy) methyl) piperidin-3-yl)-1-oxoisoindolin-4-yl) prop-2-yn-1-yl)-3-methyl-1H-indazole-6-carboxamide (Intermediate 307, 0.73 g, 1.26 mmol) in 1,4-dioxane (20 mL) was added tert-butyl (R)-6-bromo-2-(1-methylpyrrolidin-2-yl)-1H-pyrrolo[3,2-c]pyridine-1-carboxylate (Intermediate 1, 0.60 g, 1.57 mmol) followed by cesium carbonate (1.55 g, 4.73 mmol). The reaction mixture was purged with argon for 15 min, then Pd₂(dba)₃ (0.22 g, 0.23 mmol) and Xantphos (0.28 g, 0.47 mmol) were added. The reaction mixture was purged with argon for another 10 min and then stirred at 100° C. for 2 h. The reaction mixture was concentrated in vacuo and the crude residue purified by combi-flash chromatography, by eluting with 5% MeOH in dichloromethane, to afford tert-butyl-6-(1-(3-(2-(2,6-dioxo-1-((2-(trimethylsilyl)ethoxy)methyl)piperidin-3-yl)-1-oxoisoindolin-4-yl)prop-2-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 308, 0.90 g, 82%) as a yellow solid. ¹H NMR (400 MHz, DMSO-d₆) δ ppm 0.02 (s, 9H), 0.78-0.88 (m, 2H), 1.62-1.68 (m, 2H), 1.77 (s, 9H), 1.84-1.89 (m, 2H), 1.97-2.11 (m, 1H), 2.23-2.30 (m, 1H), 2.36-2.40 (m, 1H), 2.44 (s, 3H), 2.46-2.48 (m, 1H), 2.57 (s, 3H), 2.79-2.89 (m, 1H), 3.12-3.18 (m, 1H), 3.45-3.51 (m, 2H), 3.95-4.05 (m, 1H), 4.14-4.20 (m, 1H), 4.34-4.38 (m, 1H), 4.91-4.98 (m, 2H), 5.16-5.24 (m, 1H), 5.71 (s, 2H), 6.84 (s, 1H), 7.50-7.60 (m, 1H), 7.68-7.82 (m, 2H), 7.81-7.87 (m, 2H), 8.68 (s, 1H), 8.73 (s, 1H), 9.05 (s, 1H), 10.90 (br s, 1H). Mass spec m/z 884.8 [M+H]⁺.

### Step 6

### Example 72: 1-(3-(2-(2,6-dioxopiperidin-3-yl)-1-oxoisoindolin-4-yl)prop-2-yn-1-yl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(3-(2-(2,6-dioxo-1-((2-(trimethylsilyl)ethoxy)methyl)piperidin-3-yl)-1-oxoisoindolin-4-yl)prop-2-yn-1-yl)-3-methyl-1H-indazole-6-carboxamido)-2-((R)-1-methylpyrrolidin-2-yl)-1H-pyrrolo[3,2-c]pyridine-1-carboxylate (Intermediate 308, 0.90 g, 0.10 mmol) in acetonitrile (2.0 mL) was added methanesulfonic acid (0.06 mL, 1.01 mmol) and the reaction mixture was stirred at 60° C. for 2 h. The reaction mixture was cooled to room temperature, then N,N′-dimethylethylenediamine (0.06 mL, 0.50 mmol) followed by triethylamine (0.28 mL, 2.03 mmol) were added and the mixture stirred at room temperature for 4 h. The reaction mixture was concentrated in vacuo and the resultant residue was diluted with water (50 mL). The resultant precipitate was collected by filtration and dried in vacuo. The crude solid was purified by preparative HPLC (Method A) to afford 1-(3-(2-(2,6-dioxopiperidin-3-yl)-1-oxoisoindolin-4-yl)prop-2-yn-1-yl)-3-methyl-N-(2-((R)-1-methylpyrrolidin-2-yl)-1H-pyrrolo[3,2-c]pyridin-6-yl)-1H-indazole-6-carboxamide (Example 72, 0.01 g, 16%) as a yellow solid. ¹H NMR (400 MHz, DMSO-d₆) δ ppm 1.76-2.00 (m, 5H), 2.17 (s, 3H), 2.23-2.34 (m, 3H), 2.46-2.49 (m, 1H), 2.55 (s, 3H), 2.79-2.89 (m, 1H), 3.12-3.18 (m, 1H), 4.14-4.20 (m, 1H), 4.34-4.38 (m, 1H), 5.06-5.12 (m, 1H), 5.65 (s, 2H), 6.41 (s, 1H), 7.52 (t, J=7.60 Hz, 1H), 7.66-7.69 (m, 1H), 7.74 (d, J=7.60 Hz, 1H), 7.81-7.87 (m, 2H), 8.23 (s, 1H), 8.52 (s, 1H), 8.60 (s, 1H), 10.55 (s, 1H), 11.00 (br s, 1H), 11.40 (s, 1H). Mass spec m/z 655.1 [M+H]⁺.

### Example 73 was Synthesised Following Scheme 68

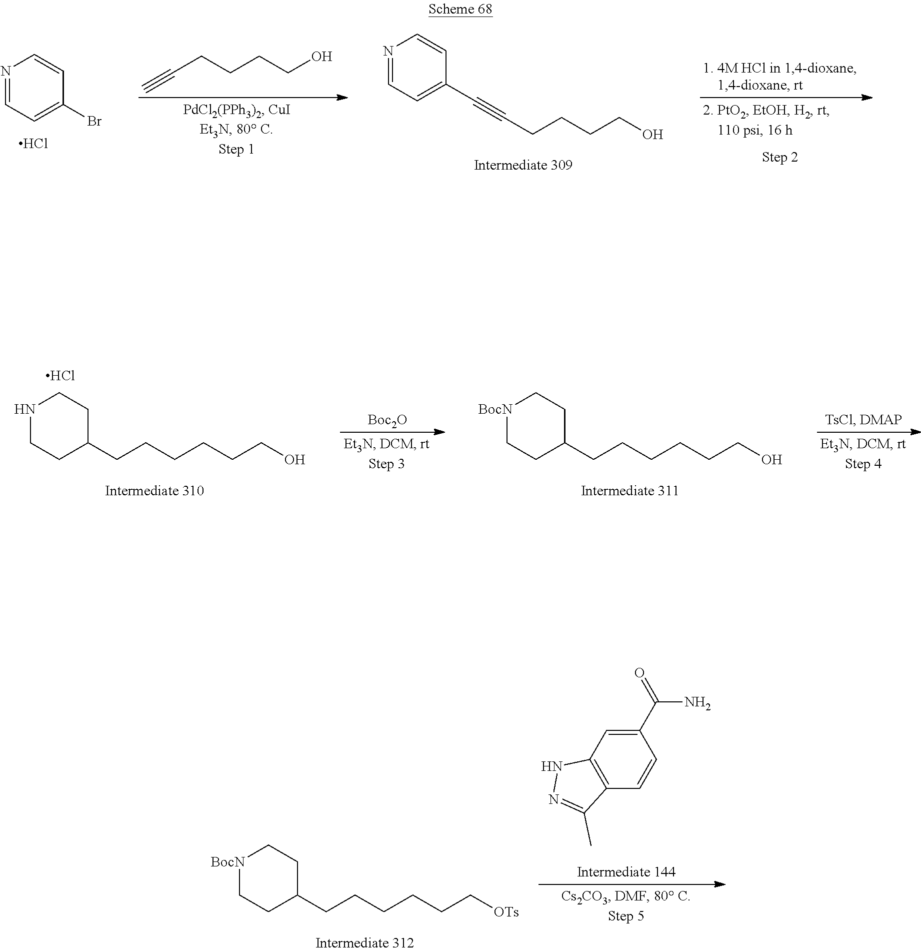

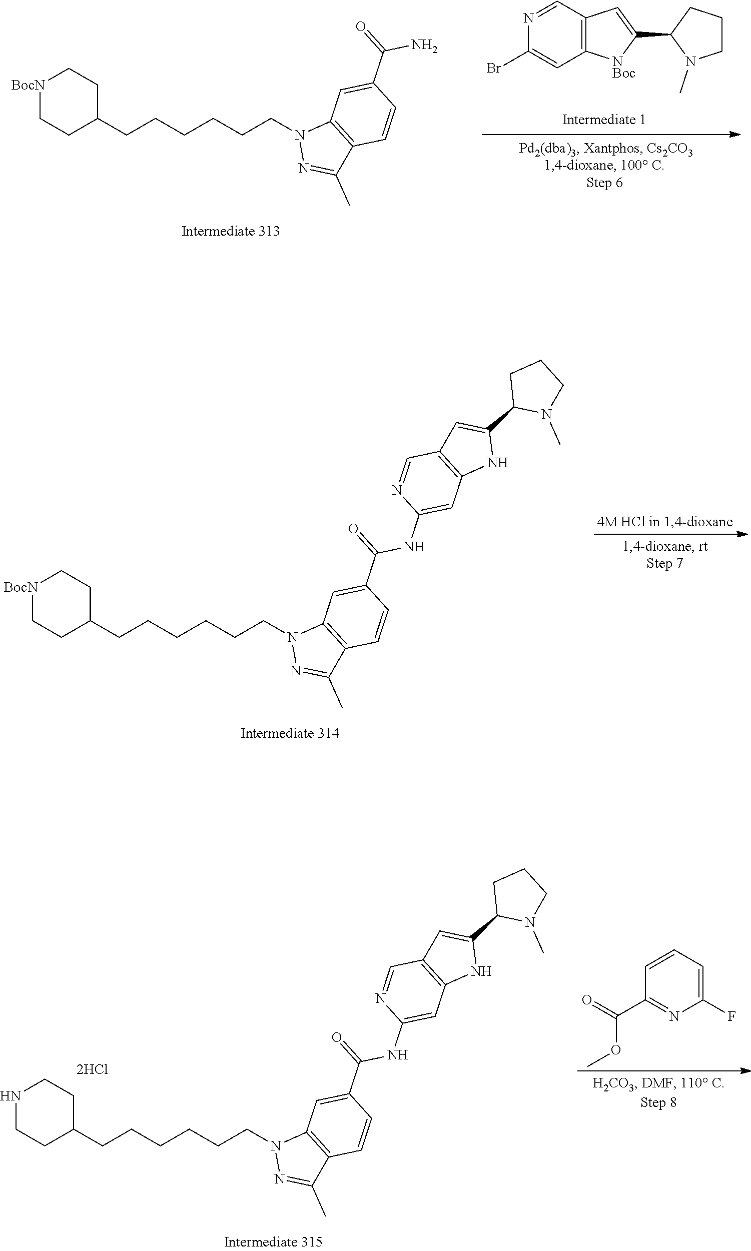 [SMILES: Cc1nn(CCCCCCC2CCN(C(=O)OC(C)(C)C)CC2)c2cc(C(N)=O)ccc12 ; CN1CCC[C@@H]1c1cc2cnc(Br)cc2n1C(=O)OC(C)(C)C ; Cc1nn(CCCCCCC2CCN(C(=O)OC(C)(C)C)CC2)c2cc(C(=O)Nc3cc4[nH]c([C@H]5CCCN5C)cc4cn3)ccc12 ; Cc1nn(CCCCCCC2CCNCC2)c2cc(C(=O)Nc3cc4[nH]c([C@H]5CCCN5C)cc4cn3)ccc12 ; COC(=O)c1cccc(F)n1]

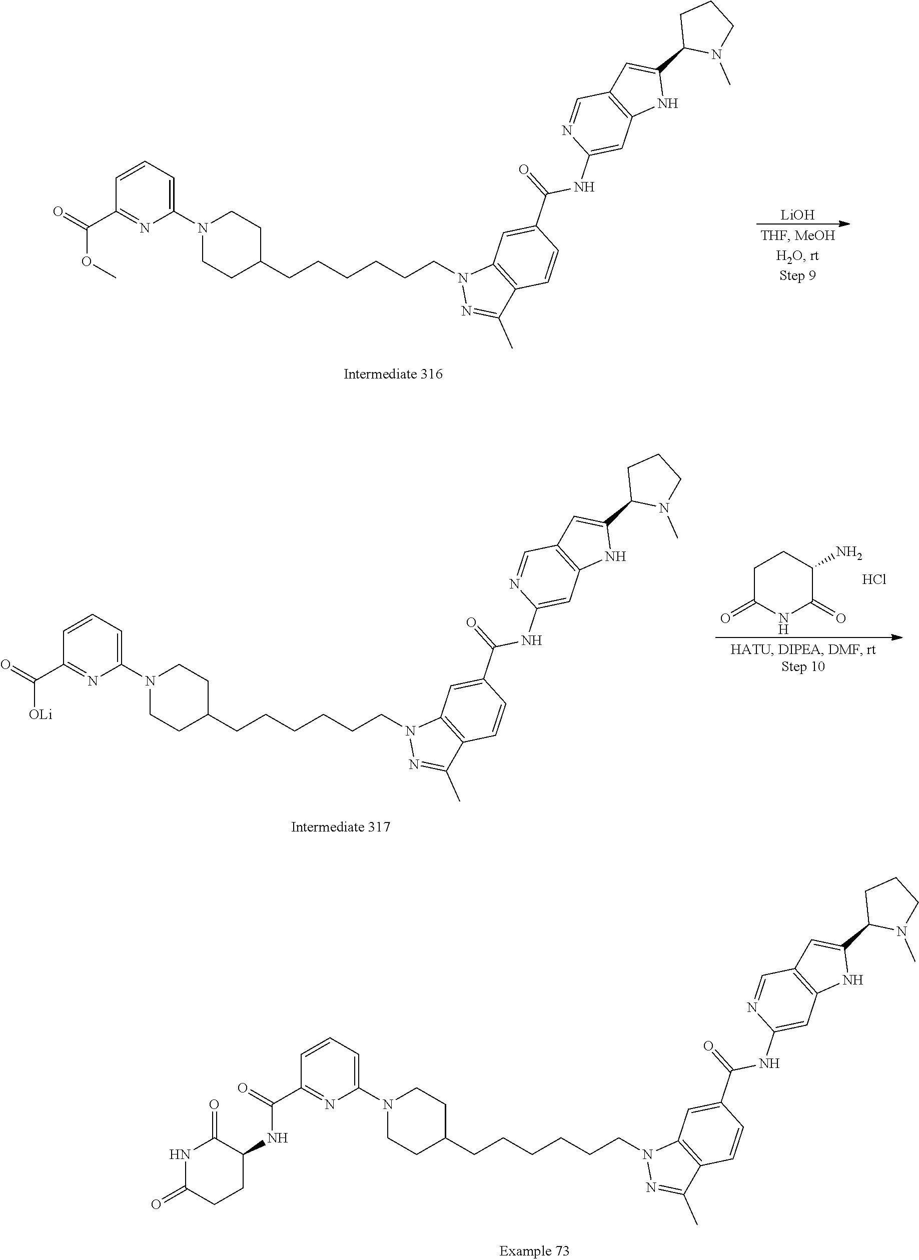

### Step 1

### Intermediate 309: 6-(pyridin-4-yl)hex-5-yn-1-ol

To a solution of 4-bromopyridine hydrochloride (CAS No: 19524-06-2, 13.0 g, 82.3 mmol) in dimethylformamide (6 mL) was added hex-5-yn-1-ol (CAS No: 928-90-5, 9.69 g, 98.7 mmol) followed by triethylamine (20 mL, 143.0 mmol) and the reaction mixture was purged with argon for 15 min. Copper(J) iodide (1.60 g, 8.23 mmol) and PdCl₂(PPh₃)₂ (5.83 g, 8.23 mmol) were added and the reaction mixture was further purged with argon for 10 min and then heated at 80° C. for 3 h. The reaction mixture was concentrated in vacuo. The obtained residue was diluted with aqueous saturated sodium thiosulfate solution (200 mL) and extracted with ethyl acetate (3×500 mL). The combined organic phases were dried over anhydrous Na₂SO₄, filtered, and concentrated in vacuo. The crude material was purified by combi-flash chromatography by eluting with 100% ethyl acetate in heptane to afford 6-(pyridin-4-yl) hex-5-yn-1-ol (Intermediate 309, 8.00 g, 55%) as a yellow liquid. ¹H NMR (400 MHz, DMSO-d₆) δ ppm 1.48-1.61 (m, 4H), 2.48 (t, J=7.20 Hz, 2H), 3.34-3.45 (m, 2H), 4.39-4.46 (m, 1H), 7.32-7.38 (m, 2H), 7.54-7.64 (m, 2H). Mass spec: m/z: 176.0 [M+H]⁺.

### Step 2

### Intermediate 310: 6-(piperidin-4-yl)hexan-1-ol hydrochloride

To a solution of 6-(pyridin-4-yl)hex-5-yn-1-ol (Intermediate 309, 10.0 g, 57.1 mmol) in 1,4-dioxane (5 mL) was added 4M hydrochloric acid in 1,4-dioxane (5 mL) at room temperature and the reaction stirred for 30 min. The reaction mixture was concentrated in vacuo and the obtained residue was dissolved in ethanol (5.0 mL, 86.0 mmol) platinum(IV)oxide (2.59 g, 11.4 mmol) was added and the reaction mixture was stirred at room temperature for 16 h under H₂ atmosphere at 110 psi. The reaction mixture was filtered through celite, washed with methanol (100 mL) and the filtrate was concentrated in vacuo to afford 6-(piperidin-4-yl)hexan-1-ol hydrochloride (Intermediate 310, 8.60 g, 81%) as a yellow liquid, which was used for the next step without further purification. ¹H NMR (400 MHz, DMSO-d₆) δ ppm 1.14-1.54 (m, 13H), 1.72-1.76 (m, 2H), 2.72-2.80 (m, 2H), 3.12-3.20 (m, 2H), 3.34-3.38 (m, 2H), 3.89 (br s, 1H), 8.89 (br s, 1H), 9.12 (br s, 1H). Mass spec: m/z: 186.3 [M+H]⁺.

### Step 3

### Intermediate 311: tert-butyl 4-(6-hydroxyhexyl) piperidine-1-carboxylate

To a solution of 6-(piperidin-4-yl)hexan-1-ol hydrochloride (Intermediate 310, 8.60 g, 46 mmol) in dichloromethane (5 mL) was added triethylamine (33 mL, 230 mmol) followed by di-tert-butyl dicarbonate (16 mL, 70 mmol). The reaction mixture was stirred at room temperature for 7 h under a nitrogen atmosphere. The reaction mixture was quenched with water (30 mL) and extracted with dichloromethane (2×30 mL). The combined organic phases were dried over anhydrous Na₂SO₄, filtered, and concentrated in vacuo to afford tert-butyl 4-(6-hydroxyhexyl)piperidine-1-carboxylate (Intermediate 311, 8.00 g, 60%)) as a yellow liquid, which was used for the next step without further purification. ¹H NMR (400 MHz, DMSO-d₆) δ ppm 0.84-0.94 (m, 2H), 1.06-1.24 (m, 8H), 1.26-1.31 (m, 1H), 1.38 (s, 9H), 1.55-1.65 (m, 4H), 2.58-2.74 (m, 2H), 3.34-3.40 (m, 2H), 3.86-3.94 (m, 2H), 4.29-4.32 (m, 1H). Mass spec: m/z: 186.23 [M+H]⁺.

### Step 4

### Intermediate 312: tert-butyl 4-(6-(tosyloxy)hexyl) piperidine-1-carboxylate

To a solution of tert-butyl 4-(6-hydroxyhexyl)piperidine-1-carboxylate (Intermediate 311, 8.00 g, 28.0 mmol) in dichloromethane (100 mL) was added triethylamine (19.5 mL, 140.0 mmol) and 4-toluenesulfonyl chloride (8.02 g, 42.0 mmol) followed by 4-dimethylaminopyridine (0.36 g, 2.80 mmol) at 0° C. The reaction mixture was stirred at room temperature for 16 h under nitrogen atmosphere. The reaction mixture was quenched with water (300 mL) and extracted with dichloromethane (2×300 mL). The combined organic phases were dried over anhydrous Na₂SO₄ filtered, and concentrated in vacuo. The crude material was purified by combi-flash chromatography by eluting with 50% ethyl acetate in heptane to afford tert-butyl 4-(6-(tosyloxy)hexyl)piperidine-1-carboxylate (Intermediate 312, 9.00 g, 73%) as a yellow liquid. ¹H NMR (400 MHz, DMSO-d₆) δ ppm 0.84-0.94 (m, 2H), 1.06-1.24 (m, 8H), 1.26-1.31 (m, 1H), 1.38 (s, 9H), 1.49-1.63 (m, 4H), 2.42 (s, 3H), 2.58-2.72 (m, 2H), 3.85-3.95 (m, 2H), 3.96-4.05 (m, 2H), 7.43-7.52 (m, 2H), 7.74-7.82 (m, 2H). Mass spec: m/z: 439.9 [M+H]⁺.

### Step 5

### Intermediate 313: tert-butyl 4-(6-(6-carbamoyl-3-methyl-1H-indazol-1-yl)hexyl)piperidine-1-carboxylate

To a solution of tert-butyl 4-(6-(tosyloxy)hexyl)piperidine-1-carboxylate (Intermediate 312, 7.50 g, 17.0 mmol) in dimethylformamide (50 mL) was added 3-methyl-1H-indazole-6-carboxamide (Intermediate 144, 3.0 g, 17.0 mmol) followed by cesium carbonate (17 g, 51.0 mmol) at room temperature and the reaction mixture was heated at 80° C. for 5 h. The reaction mixture was quenched with water (300 mL) and extracted with ethyl acetate (2×300 mL). The combined organic phases were dried over anhydrous Na₂SO₄, filtered and concentrated in vacuo to get the crude mixture which was separated by combi-flash chromatography by eluting with 90-95% ethyl acetate in heptane to afford tert-butyl 4-(6-(6-carbamoyl-3-methyl-1H-indazol-1-yl)hexyl)piperidine-1-carboxylate (Intermediate 313, 3.50 g, 46%) as a yellow liquid. ¹H NMR (400 MHz, DMSO-d₆) δ ppm 0.86-0.94 (m, 2H), 1.08-1.34 (m, 6H), 1.38 (s, 9H), 1.54-1.60 (m, 2H), 1.76-1.86 (m, 2H), 2.51 (s, 3H), 2.60-2.68 (m, 2H), 3.16-3.29 (m, 3H), 3.86-3.92 (m, 2H), 4.33 (t, J=6.40 Hz, 2H), 7.32 (br s, 1H), 7.60 (d, J=8.29 Hz, 1H), 7.72 (d, J=8.29 Hz, 1H), 7.96 (br s, 1H), 8.11 (br s, 1H). Mass spec: m/z: 443.0 [M+H]⁺.

### Step 6

### Intermediate 314: tert-butyl (R)-6-(1-(6-(1-(tert-butoxycarbonyl)piperidin-4-yl)hexyl)-3-methyl-1H-indazole-6-carboxamido)-2-(1-methylpyrrolidin-2-yl)-1H-pyrrolo[3,2-c]pyridine-1-carboxylate

To a solution of tert-butyl 4-(6-(6-carbamoyl-3-methyl-1H-indazol-1-yl)hexyl)piperidine-1-carboxylate (Comp-Intermediate 313, 2.33 g, 5.26 mmol) in 1,4-dioxane (50 mL) was added tert-butyl (R)-6-bromo-2-(1-methylpyrrolidin-2-yl)-1H-pyrrolo[3,2-c]pyridine-1-carboxylate (Intermediate 1, 2.50 g, 6.57 mmol) followed by cesium carbonate (6.43 g, 19.7 mmol) at room temperature. The reaction mixture was purged with argon for 15 min then Pd₂(dba)₃ (0.93 g, 0.98 mmol) and Xantphos (1.18 g, 1.97 mmol) were added. The reaction mixture was further purged with argon for 10 min and heated at 100° C. for 2h. Progress of the reaction was monitored by TLC. After completion, the reaction mixture was filtered through a celite bed, washed with ethyl acetate (50 mL) and the filtrate was concentrated in vacuo. The crude material was purified by combi-flash chromatography by eluting with 95% ethyl acetate in heptane to afford tert-butyl (R)-6-(1-(6-(1-(tert-butoxycarbonyl)piperidin-4-yl)hexyl)-3-methyl-1H-indazole-6-carboxamido)-2-(1-methylpyrrolidin-2-yl)-1H-pyrrolo[3,2-c]pyridine-1-carboxylate (Intermediate 314, 1.10 g, 23%) as a yellow solid. ¹H NMR (400 MHz, DMSO-d₆) δ ppm 0.82-0.92 (m, 2H), 1.08-1.33 (m, 9H), 1.35 (s, 9H), 1.50-1.57 (m, 3H), 1.59-1.66 (m, 1H), 1.71 (s, 9H), 1.74-1.89 (m, 5H), 2.29-2.34 (m, 1H), 2.36 (s, 3H), 2.38-2.43 (m, 1H), 2.52 (s, 3H), 3.11-3.17 (m, 1H), 3.80-3.95 (m, 3H), 4.36-4.44 (m, 2H), 6.77 (s, 1H), 7.71-7.75 (m, 2H), 7.77-7.81 (m, 1H), 8.62 (s, 1H), 8.99 (s, 1H), 10.76 (s, 1H). Mass spec: m/z: 742.3 [M+H]⁺.

### Step 7

### Intermediate 315: (R)-3-methyl-N-(2-(1-methylpyrrolidin-2-yl)-1H-pyrrolo[3,2-c]pyridin-6-yl)-1-(6-(piperidin-4-yl) hexyl)-1H-indazole-6-carboxamide dihydrochloride

To a solution of tert-butyl (R)-6-(1-(6-(1-(tert-butoxycarbonyl) piperidin-4-yl) hexyl)-3-methyl-1H-indazole-6-carboxamido),-2-(1-methylpyrrolidin-2-yl)-1H-pyrrolo[3,2-c]pyridine-1-carboxylate (Intermediate 314, 1.10 g, 1.6 mmol) in 1,4-dioxane (10 mL) was added 4M hydrochloric acid in 1,4-dioxane (20 mL, 80.0 mmol) at room temperature and the reaction mixture was stirred at room temperature under nitrogen atmosphere for 16 h. The reaction mixture was concentrated in vacuo to afford (R)-3-methyl-N-(2-(1-methylpyrrolidin-2-yl)-1H-pyrrolo[3,2-c]pyridin-6-yl)-1-(6-(piperidin-4-yl),hexyl)-1H-indazole-6-carboxamide dihydrochloride (Intermediate 315, 1.00 g, 91%) as an off white solid, which was used for the next step without further purification. ¹H NMR (400 MHz, DMSO-d₆) δ ppm 1.10-1.35 (m, 10H), 1.42-1.50 (m, 1H), 1.68-1.76 (m, 2H), 1.80-1.92 (m, 2H), 2.13-2.25 (m, 2H), 2.38-2.42 (m, 1H), 2.49 (s, 3H), 2.55 (s, 3H), 2.56-2.58 (m, 1H), 2.66-2.80 (m, 2H), 3.12-3.20 (m, 2H), 3.80-3.95 (m, 2H), 4.42-4.46 (m, 2H), 4.74-4.80 (m, 1H), 7.29 (s, 1H), 7.81-7.93 (m, 2H), 8.44 (s, 1H), 8.59 (s, 1H), 8.76-8.91 (m, 2H), 9.13 (s, 1H), 11.48 (br s, 1H), 12.30 (br s, 1H), 13.29 (br s, 1H). Mass spec: m/z: 542 [M+H]⁺.

### Step 8

### Intermediate 316: methyl (R)-6-(4-(6-(3-methyl-6-((2-(1-methylpyrrolidin-2-yl)-1H-pyrrolo[3,2-c]pyridin-6-yl)carbamoyl)-1H-indazol-1-yl)hexyl)piperidin-1-yl)picolinate

To a solution of (R)-3-methyl-N-(2-(1-methylpyrrolidin-2-yl)-1H-pyrrolo[3,2-c]pyridin-6-yl)-1-(6-(piperidin-4-yl)hexyl)-1H-indazole-6-carboxamide dihydrochloride (Intermediate 315, 1.00 g, 1.8 mmol) in dimethylformamide (15 mL) were added methyl 6-fluoropicolinate (CAS No: 455-71-0, 0.29 g, 1.8 mmol) and potassium carbonate (0.77 g, 5.5 mmol) and the reaction mixture was heated at 110° C. for 16h. Water (100 mL) was added and a solid precipitated, which was filtered and dried under vacuum to afford methyl (R)-6-(4-(6-(3-methyl-6-((2-(1-methylpyrrolidin-2-yl)-1H-pyrrolo[3,2-c]pyridin-6-yl)carbamoyl)-1H-indazol-1-yl)hexyl)piperidin-1-yl)picolinate (Intermediate 316, 0.70 g, 60%) as a brown solid, which was used for the next step without further purification. ¹H NMR (400 MHz, DMSO-d₆) δ ppm 0.98-1.08 (m, 2H), 1.14-1.35 (m, 9H), 1.38-1.48 (m, 1H), 1.64-1.72 (m, 2H), 1.76-1.97 (m, 5H), 2.12-2.14 (m, 1H), 2.17 (s, 3H), 2.23-2.31 (m, 1H), 2.51 (s, 3H), 2.67-2.79 (m, 2H), 3.10-3.19 (m, 1H), 3.81 (s, 3H), 4.24-4.34 (m, 2H), 4.36-4.44 (m, 2H), 6.40 (s, 1H), 6.97-6.99 (m, 1H), 7.21-7.23 (m, 1H), 7.58-7.62 (m, 1H), 7.70-7.81 (m, 2H), 8.25 (s, 1H), 8.44 (s, 1H), 8.52 (s, 1H), 10.52 (br s, 1H), 11.37 (br s, 1H). Mass spec: m/z: 677.0 [M+H]⁺.

### Step 9

### Intermediate 317: Lithium salt of (R)-6-(4-(6-(3-methyl-6-((2-(1-methylpyrrolidin-2-yl)-1H-pyrrolo[3,2-c]pyridin-6-yl)carbamoyl)-1H-indazol-1-yl)hexyl)piperidin-1-yl)picolinic acid

To a solution of methyl (R)-6-(4-(6-(3-methyl-6-((2-(1-methylpyrrolidin-2-yl)-1H-pyrrolo[3,2-c]pyridin-6-yl)carbamoyl)-1H-indazol-1-yl)hexyl)piperidin-1-yl)picolinate (Intermediate 316, 0.70 g, 1.0 mmol) in tetrahydrofuran (10 mL), methanol (10 mL) and water (5 mL) was added lithium hydroxide (0.05 g, 2.0 mmol) and the reaction mixture was stirred at room temperature for 5 h. The reaction mixture was directly concentrated in vacuo to afford as lithium salt of (R)-6-(4-(6-(3-methyl-6-((2-(1-methylpyrrolidin-2-yl)-1H-pyrrolo[3,2-c]pyridin-6-yl)carbamoyl)-1H-indazol-1-yl)hexyl)piperidin-1-yl)picolinic acid (Intermediate 317, 0.50 g, 70%) as a yellow solid, which was used for the next step without further purification. ¹H NMR (400 MHz, DMSO-d₆) δ ppm 0.93-1.06 (m, 2H), 1.07-1.34 (m, 9H), 1.52-1.60 (m, 2H), 1.74-1.94 (m, 6H), 2.12-2.14 (m, 1H), 2.16 (s, 3H), 2.21-2.29 (m, 1H), 2.52 (s, 3H), 2.59-2.68 (m, 2H), 3.11-3.18 (m, 1H), 4.02-4.08 (m, 2H), 4.38-4.44 (m, 2H), 6.35 (s, 1H), 6.74-6.80 (m, 1H), 7.21-7.27 (m, 1H), 7.47-7.54 (m, 1H), 7.72-7.78 (m, 2H), 8.14 (s, 1H), 8.41 (s, 1H), 8.44 (s, 1H), 8.54 (br s, 1H), 10.61 (br s, 1H). Mass spec: m/z: 663.0 [M+H]⁺.

### Step 10

### Example 73: 1-(6-(1-(6-(((S)-2,6-dioxopiperidin-3-yl)carbamoyl)pyridin-2-yl)piperidin-4-yl)hexyl)-3-methyl-N-(2-((R)-1-methylpyrrolidin-2-yl)-1H-pyrrolo[3,2-c]pyridin-6-yl)-1H-indazole-6-carboxamide

To a solution of the lithium salt of (R)-6-(4-(6-(3-methyl-6-((2-(1-methylpyrrolidin-2-yl)-1H-pyrrolo[3,2-c]pyridin-6-yl)carbamoyl)-1H-indazol-1-yl)hexyl)piperidin-1-yl)picolinic acid (Intermediate 317, 0.50 g, 0.7 mmol) in dimethylformamide (15 mL) was added HATU (0.4 g, 0.9 mmol) and the reaction stirred at room temperature for 15 min. (S)-3-aminopiperidine-2,6-dione hydrochloride (CAS No: 25181-50-4, 0.1 g, 0.7 mmol) and N,N-diisopropylethylamine (0.6 mL, 4 mmol) were added and the reaction mixture was stirred at room temperature for 16h. The reaction mixture was poured into ice cold water (100 mL) to get a precipitate which was filtered and dried in vacuo to obtain the crude compound. The crude material was purified through preparative HPLC (Method A) to afford 1-(6-(1-(6-(((S)-2,6-dioxopiperidin-3-yl)carbamoyl)pyridin-2-yl)piperidin-4-yl)hexyl)-3-methyl-N-(2-((R)-1-methylpyrrolidin-2-yl)-1H-pyrrolo[3,2-c]pyridin-6-yl)-1H-indazole-6-carboxamide (Example 73, 0.21 g, 40%) as an off white solid. ¹H NMR (400 MHz, DMSO-d₆) δ ppm 1.00-1.11 (m, 2H), 1.14-1.35 (m, 9H), 1.38-1.48 (m, 1H), 1.65-1.74 (m, 2H), 1.80-2.04 (m, 6H), 2.12-2.14 (m, 1H), 2.17 (s, 3H), 2.22-2.31 (m, 2H), 2.52 (s, 3H), 2.53-2.55 (m, 1H), 2.70-2.81 (m, 3H), 3.11-3.18 (m, 1H), 4.32-4.43 (m, 4H), 4.67-4.76 (m, 1H), 6.40 (s, 1H), 6.96 (d, J=8.80 Hz, 1H), 7.24 (d, J=7.20 Hz, 1H), 7.62 (t, J=7.20 Hz, 1H), 7.71-7.81 (m, 2H), 8.25 (s, 1H), 8.44 (s, 1H), 8.52 (s, 1H), 8.71 (d, J=8.80 Hz, 1H), 10.53 (s, 1H), 10.87 (s, 1H), 11.38 (s, 1H). Mass spec: m/z: 773.2 [M+H]⁺.

### Example 74 was Synthesised Following Scheme 69

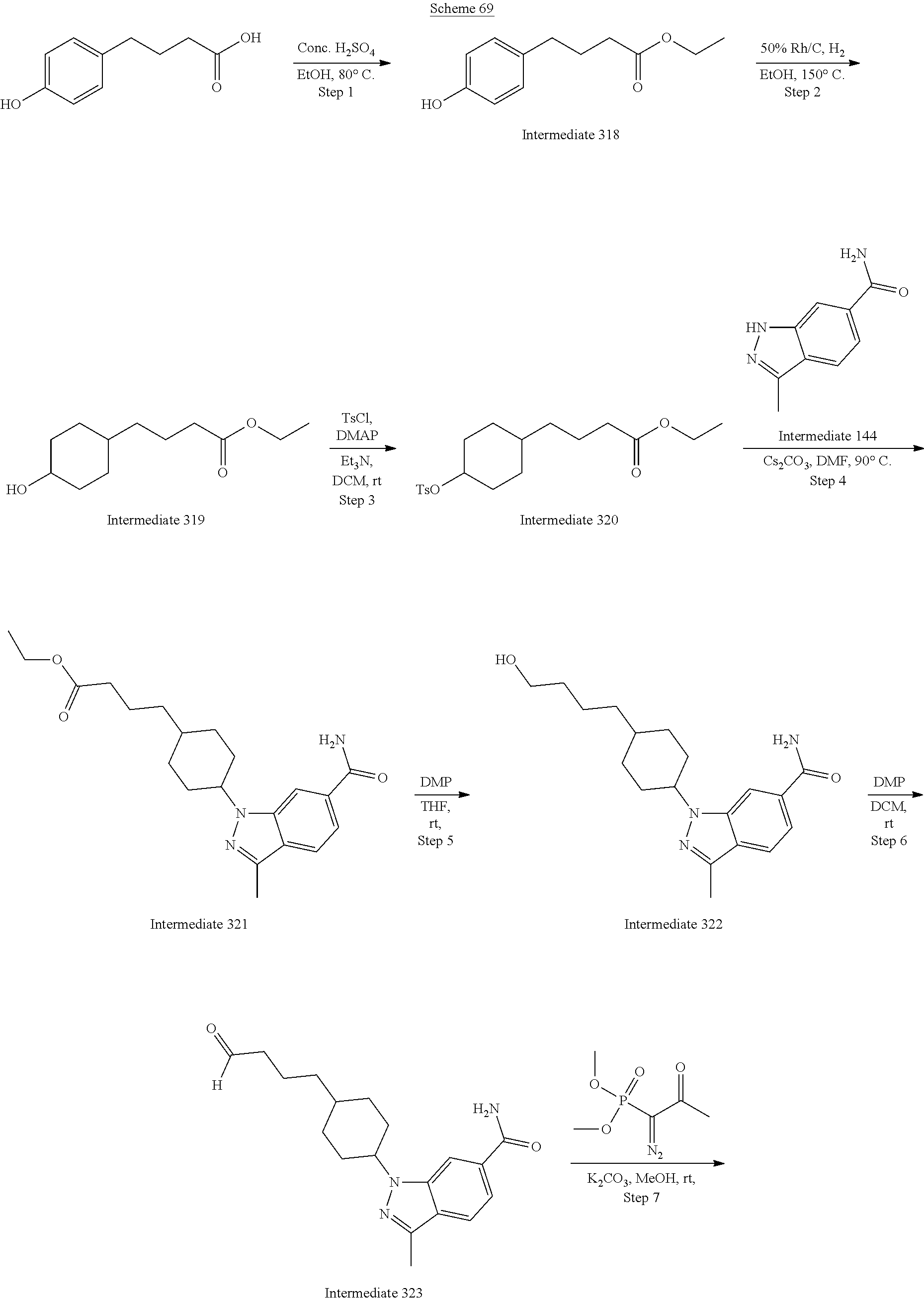

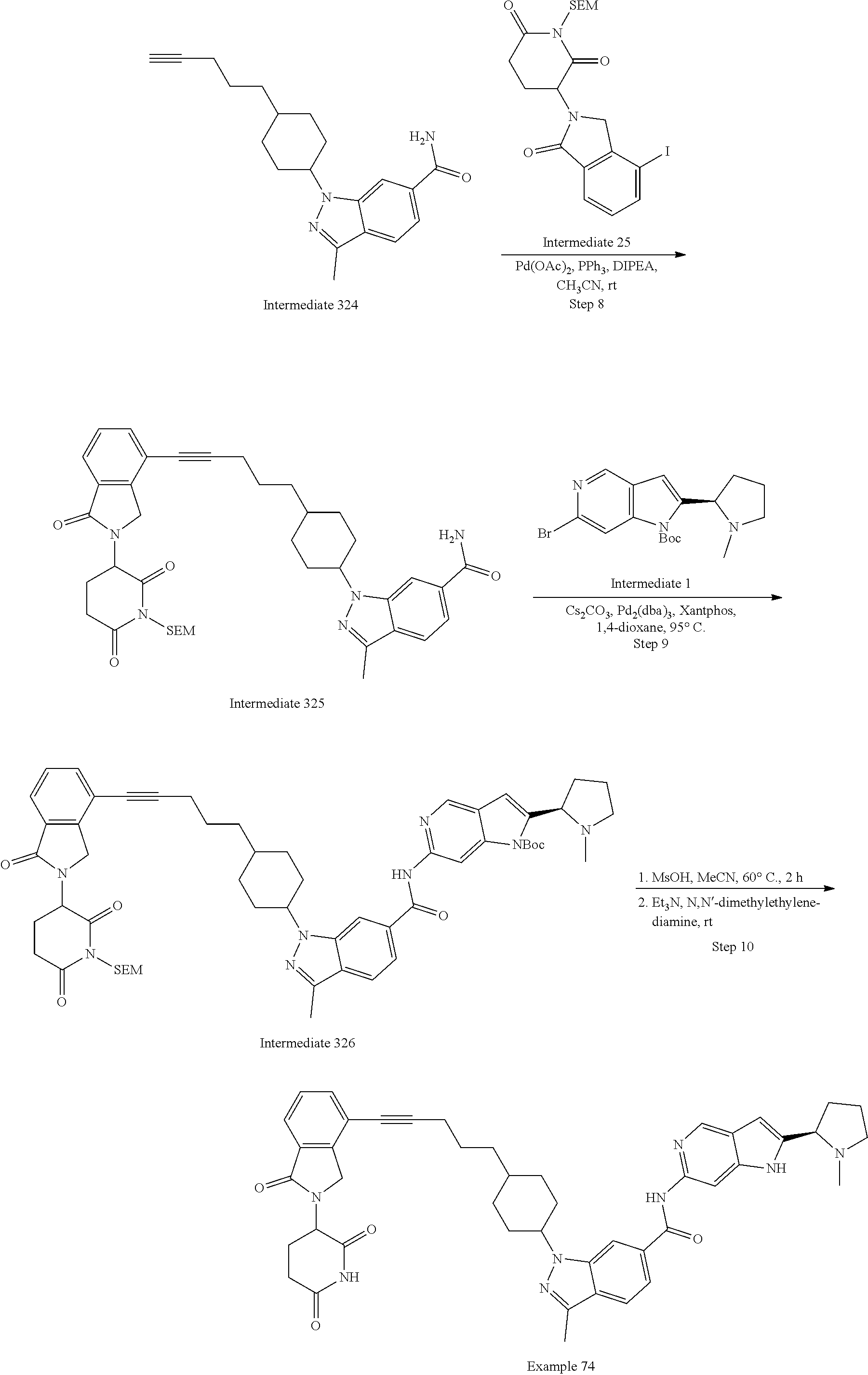 [SMILES: C#CCCCC1CCC(n2nc(C)c3ccc(C(N)=O)cc32)CC1 ; C[Si](C)(C)CCOCN1C(=O)CCC(N2Cc3c(I)cccc3C2=O)C1=O ; Cc1nn(C2CCC(CCCC#Cc3cccc4c3CN(C3CCC(=O)N(COCC[Si](C)(C)C)C3=O)C4=O)CC2)c2cc(C(N)=O)ccc12 ; CN1CCC[C@@H]1c1cc2cnc(Br)cc2n1C(=O)OC(C)(C)C ; Cc1nn(C2CCC(CCCC#Cc3cccc4c3CN(C3CCC(=O)N(COCC[Si](C)(C)C)C3=O)C4=O)CC2)c2cc(C(=O)Nc3cc4c(cn3)cc([C@H]3CCCN3C)n4C(=O)OC(C)(C)C)ccc12 ; Cc1nn(C2CCC(CCCC#Cc3cccc4c3CN(C3CCC(=O)NC3=O)C4=O)CC2)c2cc(C(=O)Nc3cc4[nH]c([C@H]5CCCN5C)cc4cn3)ccc12]

### Step 1

### Intermediate 318: ethyl 4-(4-hydroxyphenyl)butanoate

To a solution of 4-(4-hydroxyphenyl)butanoic acid (CAS No: 7021-11-6, 25.0 g, 138.73 mmol) in ethanol (250 mL) was added sulfuric acid (6.80 g, 69.36 mmol) at 0° C. and the reaction mixture was heated at 80° C. for 2 h. The reaction mixture was concentrated in vacuo to obtain crude compound. The crude material was purified by combi-flash chromatography by eluting with 35% ethyl acetate in heptane to afford ethyl 4-(4-hydroxyphenyl) butanoate (Intermediate 318, 23.0 g, 80%) as a liquid. ¹H NMR (400 MHz, DMSO-d₆) δ ppm 1.19 (t, J=7.20 Hz, 3H), 1.73-1.80 (m, 2H), 2.26 (t, J=7.60 Hz, 2H), 2.46 (t, J=7.60 Hz, 2H), 4.06 (q, J=7.20 Hz, 2H), 6.67 (d, J=8.40 Hz, 2H), 6.97 (d, J=8.40 Hz, 2H), 9.14 (s, 1H). Mass spec: m/z: 209.10 [M+H]⁺.

### Step 2

### Intermediate 319: ethyl 4-(4-hydroxycyclohexyl)butanoate

To a solution of ethyl 4-(4-hydroxyphenyl) butanoate (Intermediate 318, 15.0 g, 72.03 mmol) in ethanol (150 mL) was added 50% Rh/C (0.5 mL, 30.0 mmol) at room temperature under a nitrogen atmosphere. The atmosphere was then changed to hydrogen and the reaction mixture was heated at 150° C. for 72 h under a hydrogen atmosphere at 180 psi. The reaction mixture was filtered through celite, washed with methanol (100 mL) and concentrated in vacuo. The crude material was purified by combi-flash column chromatography by eluting with 30% ethyl acetate in heptane to afford ethyl 4-(4-hydroxycyclohexyl) butanoate (Intermediate 319, 10.0 g, 65%) as a yellow liquid.

¹H NMR (400 MHz, DMSO-d₆) δ ppm 0.79-0.93 (m, 1H), 1.03-1.07 (m, 2H), 1.11-1.23 (m, 2H), 1.17 (t, J=7.20 Hz, 3H), 1.27-1.43 (m, 3H), 1.45-1.59 (m, 3H), 1.62-1.68 (m, 1H), 1.76-1.80 (m, 1H), 2.19-2.29 (m, 2H), 3.26-3.32 (m, 1H), 3.71 (br s, 1H), 4.05 (q, J=7.20 Hz, 2H).

### Step 3

### Intermediate 320: ethyl 4-(4-(tosyloxy)cyclohexyl)butanoate

To a solution of ethyl 4-(4-hydroxycyclohexyl)butanoate (Intermediate 319, 9.00 g, 42.0 mmol) in dichloromethane (100 mL) was added triethylamine (20.0 mL, 130.0 mmol) and 4-dimethylaminopyridine (2.7 g, 21.0 mmol) at 0° C. After stirring for 5 min, p-toluenesulfonyl chloride (9.4 mL, 63.0 mmol) was added and the reaction stirred at room temperature for 16h. The reaction mixture was poured in ice cold water (120 mL) and extracted with dichloromethane (2×150 mL). The combined organic phases were dried over anhydrous Na₂SO₄, filtered, and concentrated in vacuo. The crude material was purified by combi-flash chromatography by eluting with 50% ethyl acetate in heptane to afford ethyl 4-(4-(tosyloxy)cyclohexyl)butanoate (Intermediate 320, 10.0 g, 65%) as a yellow liquid. ¹H NMR (400 MHz, DMSO-d₆) δ ppm 0.82-0.96 (m, 1H), 1.05-1.12 (m, 4H), 1.13 (t, J=7.20 Hz, 3H), 1.34-1.55 (m, 5H), 1.62-1.66 (m, 2H), 1.74-1.78 (m, 1H), 2.20-2.25 (m, 2H), 2.41 (s, 3H), 4.02 (q, J=7.20 Hz, 2H), 4.31-4.39 (m, 1H), 7.46 (d, J=7.80 Hz, 2H), 7.78 (d, J=7.80 Hz, 2H).

### Step 4

### Intermediate 321: ethyl 4-(4-(6-carbamoyl-3-methyl-1H-indazol-1-yl) cyclohexyl)butanoate

To a solution of ethyl 4-(4-(tosyloxy) cyclohexyl)butanoate (Intermediate 320, 9.6 g, 26.0 mmol) in dimethylformamide (70 mL) was added 3-methyl-1H-indazole-6-carboxamide (Intermediate 144, 3.50 g, 20.0 mmol) followed by cesium carbonate (17.0 g, 50.0 mmol) and the reaction mixture was heated at 90° C. for 5 h. The reaction mixture was then poured into ice cold water (100 mL) and extracted with ethyl acetate (3×120 mL). combined organic phases were dried over anhydrous Na₂SO₄, filtered, and concentrated in vacuo to get the crude which was purified by combi-flash chromatography by eluting with 70% ethyl acetate in heptane to afford ethyl 4-(4-(6-carbamoyl-3-methyl-1H-indazol-1-yl) cyclohexyl) butanoate (Intermediate 321, 2.50 g, 34%) as a yellow liquid. ¹H NMR (400 MHz, DMSO-d₆) δ ppm 1.19 (t, J=7.60 Hz, 3H), 1.24-1.31 (m, 1H), 1.40-1.48 (m, 1H), 1.54-1.64 (m, 2H), 1.67-1.79 (m, 4H), 1.84-1.99 (m, 2H), 1.99-2.12 (m, 1H), 2.29-2.35 (m, 2H), 2.36 (s, 3H), 2.74-2.76 (m, 1H), 2.89-2.92 (m, 1H), 4.12 (q, J=7.60 Hz, 2H), 4.51-4.64 (m, 1H), 7.42 (br s, 1H), 7.59-7.62 (m, 1H), 7.73-7.75 (m, 1H), 8.04 (br s, 1H), 8.15-8.17 (m, 1H). Mass spec: m/z: 372.64 [M+H]⁺.

### Step 5

### Intermediate 322: 1-(4-(4-hydroxybutyl) cyclohexyl)-3-methyl-1H-indazole-6-carboxamide

To a solution of ethyl 4-(4-(6-carbamoyl-3-methyl-1H-indazol-1-yl)cyclohexyl)butanoate (Intermediate 321, 2.50 g, 6.7 mmol) in tetrahydrofuran (25 mL) was added 2M Lithium borohydride solution in tetrahydrofuran (17 mL, 34.0 mmol) at 0° C. and the reaction mixture was stirred at room temperature for 32 h. The reaction mixture was poured into ice cold water (80 mL) and extracted with ethyl acetate (2×150 mL). The combined organic phases were dried over anhydrous Na₂SO₄, filtered, and concentrated in vacuo. The crude material was purified by combi-flash chromatography by eluting with ethyl acetate to afford 1-(4-(4-hydroxybutyl) cyclohexyl)-3-methyl-1H-indazole-6-carboxamide (Intermediate 322, 1.80 g, 81%) as an off white solid. ¹H NMR (400 MHz, DMSO-d₆) δ ppm 1.18-1.28 (m, 2H), 1.30-1.48 (m, 5H), 1.64-1.77 (m, 4H), 1.86-1.96 (m, 2H), 1.98-2.12 (m, 1H), 2.52 (s, 3H), 3.36-3.44 (m, 2H), 4.32-4.36 (m, 1H), 4.51-4.62 (m, 1H), 7.40 (br s, 1H), 7.59-7.62 (m, 1H), 7.70-7.74 (m, 1H), 8.03 (br s, 1H), 8.10-8.17 (m, 1H). Mass spec: m/z: 330.2 [M+H]⁺.

### Step 6

### Intermediate 323: 3-methyl-1-(4-(4-oxobutyl) cyclohexyl)-1H-indazole-6-carboxamide

To a solution of 1-(4-(4-hydroxybutyl)cyclohexyl)-3-methyl-1H-indazole-6-carboxamide (Intermediate 322, 1.1 g, 3.3 mmol) in dichloromethane (10 mL) was added Dess-Martin Periodinane (2.2 g, 5.0 mmol) at 0° C. and the reaction mixture was stirred at room temperature for 1 h. The reaction mixture was poured into saturated NaHCO₃ solution (100 mL) and extracted with dichloromethane (2×90 mL). The combined organic phases were dried over anhydrous Na₂SO₄, filtered, and concentrated in vacuo to afford 3-methyl-1-(4-(4-oxobutyl) cyclohexyl)-1H-indazole-6-carboxamide (Intermediate 323, 0.90 g, 82%) as a solid, which was used for the next step without further purification. Mass spec: m/z: 328.1 [M+H]⁺.

### Step 7

### Intermediate 324: 3-methyl-1-(4-(pent-4-yn-1-yl)cyclohexyl)-1H-indazole-6-carboxamide

To a solution of 3-methyl-1-(4-(4-oxobutyl) cyclohexyl)-1H-indazole-6-carboxamide (Intermediate 323, 0.90 g, 2.74 mmol) in methanol (10 mL) was added potassium carbonate (0.79 g, 5.49 mmol) at 0° C., followed by dimethyl (1-diazo-2-oxopropyl)phosphonate (0.79 g, 4.12 mmol), and the reaction mixture was stirred at room temperature for 2 h. The reaction mixture was poured into water (90 mL) and extracted with ethyl acetate (2×100 mL). The combined organic phases were dried over anhydrous Na₂SO₄, filtered, and concentrated in vacuo. The crude material was purified by combi-flash chromatography by eluting with 5% of MeOH in DCM to afford 3-methyl-1-(4-(pent-4-yn-1-yl)cyclohexyl)-1H-indazole-6-carboxamide (Intermediate 324, 0.31 g, 35%) as a yellow solid. ¹H NMR (400 MHz, DMSO-d₆) δ ppm 1.13-1.25 (m, 1H), 1.29-1.41 (m, 1H), 1.43-1.55 (m, 3H), 1.65-1.78 (m, 3H), 1.83-1.97 (m, 2H), 1.98-2.11 (m, 1H), 2.15-2.24 (m, 2H), 2.52 (s, 3H), 2.76-2.78 (m, 1H), 3.63-3.69 (m, 2H), 4.43-4.62 (m, 1H), 7.42 (br s, 1H), 7.57-7.60 (m, 1H), 7.71-7.73 (m, 1H), 8.04 (br s, 1H), 8.14-8.16 (m, 1H). Mass spec: m/z: 324.2 [M+H]⁺.

### Step 8

### Intermediate 325: 1-(4-(5-(2-(2,6-dioxo-1-((2-(trimethylsilyl)ethoxy)methyl)piperidin-3-yl)-1-oxoisoindolin-4-yl)pent-4-yn-1-yl)cyclohexyl)-3-methyl-1H-indazole-6-carboxamide

To a solution of 3-methyl-1-(4-(pent-4-yn-1-yl) cyclohexyl)-1H-indazole-6-carboxamide (Intermediate 324, 0.30 g, 0.93 mmol) and 3-(4-iodo-1-oxoisoindolin-2-yl)-1-((2-(trimethylsilyl)ethoxy)methyl)piperidine-2,6-dione (Intermediate 25, 0.49 g, 0.97 mmol) in acetonitrile (10 mL) was added N,N-diisopropylethylamine (0.9 mL, 4.89 mmol). The reaction mixture was purged with argon gas for 15 min. Triphenylphosphine (0.07 g, 0.29 mmol) and palladium acetate (0.02 g, 0.14 mmol) were then added and the reaction mixture was further purged with argon gas for 10 min and stirred at room temperature for 1 h. The reaction mixture was concentrated in vacuo and the resulting crude material was purified by combi-flash chromatography by eluting with 7% of MeOH in DCM to afford 1-(4-(5-(2-(2,6-dioxo-1-((2-(trimethylsilyl)ethoxy)methyl)piperidin-3-yl)-1-oxoisoindolin-4-yl)pent-4-yn-1-yl)cyclohexyl)-3-methyl-1H-indazole-6-carboxamide (Intermediate 326, 0.30 g, 44%) as a solid. ¹H NMR (400 MHz, DMSO-d₆) δ ppm −0.03 (s, 9H), 0.79-0.86 (m, 2H), 1.24-1.28 (m, 4H), 1.40-1.42 (m, 1H), 1.57-1.79 (m, 5H), 1.85-2.13 (m, 4H), 2.46 (s, 3H), 2.65-2.78 (m, 1H), 2.98-3.09 (m, 1H), 3.10-3.20 (m, 2H), 3.46-3.54 (m, 2H), 3.58-3.64 (m, 1H), 4.25-4.30 (m, 1H), 4.44-4.64 (m, 2H), 4.97-5.10 (m, 2H), 5.25-5.30 (m, 1H), 7.41 (br s, 1H), 7.53-7.60 (m, 2H), 7.66-7.68 (m, 1H), 7.70-7.74 (m, 2H), 8.03 (br s, 1H), 8.12-8.16 (m, 1H). Mass spec: m/z: 696.2 [M+H]⁺.

### Step 9

### Intermediate 326: 6-(1-(4-(5-(2-(2,6-dioxo-1-((2-(trimethylsilyl)ethoxy)methyl)piperidin-3-yl)-1-oxoisoindolin-4-yl)pent-4-yn-1-yl)cyclohexyl)-3-methyl-1H-indazole-6-carboxamido)-2-((R)-1-methylpyrrolidin-2-yl)-1H-pyrrolo[3,2-c]pyridine-1-carboxylate

To a solution of 1-(4-(5-(2-(2,6-dioxo-1-((2-(trimethylsilyl)ethoxy)methyl)piperidin-3-yl)-1-oxoisoindolin-4-yl)pent-4-yn-1-yl)cyclohexyl)-3-methyl-1H-indazole-6-carboxamide (Intermediate 325, 0.20 g, 0.29 mmol) in 1,4-dioxane (2 mL) was added tert-butyl (R)-6-bromo-2-(1-methylpyrrolidin-2-yl)-1H-pyrrolo[3,2-c]pyridine-1-carboxylate (Intermediate 1, 0.14 g, 0.36 mmol) followed by cesium carbonate (0.18 g, 0.55 mmol) at room temperature and the reaction mixture was purged with argon for 15 min followed by Pd₂(dba)₃ (0.04 g, 0.05 mmol) and Xantphos (0.06 g, 0.11 mmol). The reaction mixture was further purged with argon for 10 min and then heated at 95° C. for 1 h. The reaction mixture was concentrated in vacuo to obtain crude compound. The crude material was purified by combi-flash chromatography by eluting with 7% of MeOH in DCM to afford tert-butyl 6-(1-(4-(5-(2-(2,6-dioxo-1-((2-(trimethylsilyl)ethoxy)methyl)piperidin-3-yl)-1-oxoisoindolin-4-yl)pent-4-yn-1-yl)cyclohexyl)-3-methyl-1H-indazole-6-carboxamido)-2-((R)-1-methylpyrrolidin-2-yl)-1H-pyrrolo[3,2-c]pyridine-1-carboxylate (Intermediate 326, 0.20 g, 55%) as a solid. ¹H NMR (400 MHz, DMSO-d₆) δ ppm −0.04 (s, 9H), 0.75-0.88 (m, 2H), 1.22-1.26 (m, 1H), 1.40-1.50 (m, 2H), 1.57-1.68 (m, 8H), 1.71 (s, 9H), 1.72-1.78 (m, 4H), 2.02 (s, 3H), 2.05-2.12 (m, 2H), 2.30-2.33 (m, 2H), 2.39 (s, 3H), 2.40-2.42 (m, 1H), 2.67-2.76 (m, 1H), 2.96-3.17 (m, 2H), 3.44-3.63 (m, 3H), 3.88-3.97 (m, 1H), 3.98-4.07 (m, 1H), 4.26-4.30 (m, 1H), 4.46-4.52 (m, 1H), 4.63-4.70 (m, 1H), 5.01-5.07 (m, 2H), 5.22-5.32 (m, 1H), 6.77 (s, 1H), 7.53-7.58 (m, 1H), 7.66-7.80 (m, 4H), 8.42 (s, 1H), 8.61 (s, 1H), 8.98 (s, 1H), 10.77 (br s, 1H). Mass spec: m/z: 995.57 [M+H]⁺.

### Step 10

### Example 74: 1-(4-(5-(2-(2,6-dioxopiperidin-3-yl)-1-oxoisoindolin-4-yl) pent-4-yn-1-yl) cyclohexyl)-3-methyl-N-(2-((R)-1-methylpyrrolidin-2-yl)-1H-pyrrolo[3,2-c]pyridin-6-yl)-1H-indazole-6-carboxamide

To a solution of tert-butyl 6-(1-(4-(5-(2-(2,6-dioxo-1-((2-(trimethylsilyl)ethoxy)methyl)piperidin-3-yl)-1-oxoisoindolin-4-yl)pent-4-yn-1-yl)cyclohexyl)-3-methyl-1H-indazole-6-carboxamido)-2-((R)-1-methylpyrrolidin-2-yl)-1H-pyrrolo[3,2-c]pyridine-1-carboxylate (Intermediate 326, 0.24 g, 0.24 mmol) in acetonitrile (3 mL) was added methanesulfonic acid (0.24 g, 2.41 mmol) at room temperature and heated at 50° C. for 2h. N,N′-dimethylethylenediamine (0.13 g, 1.44 mmol) and triethylamine (0.51 g, 4.82 mmol) were added and the reaction stirred at room temperature for 2h. Progress of the reaction was monitored by TLC. After completion, the reaction mixture was concentrated in vacuo to obtain crude compound which was poured into water (50 mL), the solid precipitated out, which was filtered and dried under vacuo to get the crude compound which was purified through Prep-HPLC in acidic buffer to afford 1-(4-(5-(2-(2,6-dioxopiperidin-3-yl)-1-oxoisoindolin-4-yl)pent-4-yn-1-yl)cyclohexyl)-3-methyl-N-(2-((R)-1-methylpyrrolidin-2-yl)-1H-pyrrolo[3,2-c]pyridin-6-yl)-1H-indazole-6-carboxamide (Example 74, 0.1 g, 38%) as a white solid. ¹H NMR (400 MHz, DMSO-d₆) δ ppm 1.58-1.68 (m, 4H), 1.72-1.85 (m, 7H), 1.86-2.02 (m, 4H), 2.09-2.16 (m, 2H), 2.18 (s, 3H), 2.24-2.30 (m, 1H), 2.44-2.47 (m, 1H), 2.48 (s, 3H), 2.53-2.60 (m, 3H), 2.82-2.93 (m, 1H), 3.12-3.19 (m, 1H), 3.34-3.36 (m, 2H), 4.27-4.37 (m, 1H), 4.44-4.52 (m, 1H), 4.67-4.75 (m, 1H), 5.10-5.15 (m, 1H), 6.40 (s, 1H), 7.49-7.56 (m, 1H), 7.63-7.68 (m, 1H), 7.69-7.71 (m, 2H), 7.73-7.78 (m, 1H), 8.25 (s, 1H), 8.42 (s, 1H), 8.52 (s, 1H), 10.54 (br s, 1H), 10.98 (br s, 1H), 11.38 (br s, 1H). Mass spec: m/z: 765.0 [M+H]⁺.

### Examples 75, 76 and 77 were Synthesized Following Scheme 70

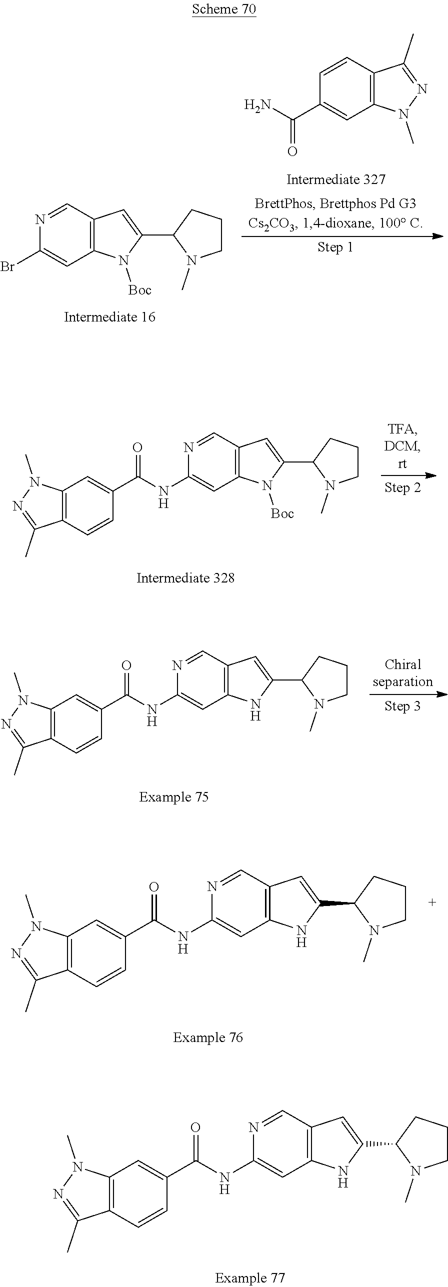 [SMILES: Cc1nn(C)c2cc(C(N)=O)ccc12 ; CN1CCCC1c1cc2cnc(Br)cc2n1C(=O)OC(C)(C)C ; Cc1nn(C)c2cc(C(=O)Nc3cc4c(cn3)cc(C3CCCN3C)n4C(=O)OC(C)(C)C)ccc12 ; Cc1nn(C)c2cc(C(=O)Nc3cc4[nH]c(C5CCCN5C)cc4cn3)ccc12 ; Cc1nn(C)c2cc(C(=O)Nc3cc4[nH]c([C@H]5CCCN5C)cc4cn3)ccc12 ; Cc1nn(C)c2cc(C(=O)Nc3cc4[nH]c([C@@H]5CCCN5C)cc4cn3)ccc12]

### Step 1

### Intermediate 328: rac-tert-butyl 6-(1,3-dimethyl-1H-indazole-6-carboxamido)-2-(1-methylpyrrolidin-2-yl)-1H-pyrrolo[3,2-c]pyridine-1-carboxylate

A solution of rac-tert-butyl 6-bromo-2-(1-methylpyrrolidin-2-yl)-1H-pyrrolo[3,2-c]pyridine-1-carboxylate (Intermediate 16, 10 g, 25.1 mmol), 1,3-dimethyl-1H-indazole-6-carboxamide (Intermediate 327: 5.98 g, 30.0 mmol), Brettphos (6.74 g, 12.6 mmol), Brettphos Pd G3 (11.4 g, 12.6 mmol) and Cs₂CO₃ (40.9 g, 125.5 mmol) in 1,4-dioxane (300 mL) was stirred for 15 h at 100° C. under nitrogen atmosphere. The reaction mixture was quenched with water and the resulting solution was extracted three times with EtOAc. The organic layers were combined, washed with brine, dried over anhydrous Na₂SO₄, filtered, and concentrated under reduced pressure. The crude material was purified by flash chromatography on silica gel using DCM/MeOH (95/5) as eluent to afford rac-tert-butyl 6-(1,3-dimethyl-1H-indazole-6-carboxamido)-2-(1-methylpyrrolidin-2-yl)-1H-pyrrolo[3,2-c]pyridine-1-carboxylate (Intermediate 328: 6 g, 42%) as a yellow oil. Mass spec: m/z 489 [M+H]⁺.

### Step 2

### Example 75: rac-1,3-dimethyl-N-(2-(1-methylpyrrolidin-2-yl)-1H-pyrrolo[3,2-c]pyridin-6-yl)-1H-indazole-6-carboxamide

To a solution of rac-tert-butyl 6-(1,3-dimethyl-1H-indazole-6-carboxamido)-2-(1-methylpyrrolidin-2-yl)-1H-pyrrolo[3,2-c]pyridine-1-carboxylate (Intermediate 328: 6 g, 11.1 mmol) in DCM (150 mL) was added TFA (30 mL). The reaction mixture was stirred for 1 h at rt. The reaction was concentrated under reduced pressure and the crude material was chromatographed by reverse-phase flash chromatography (C18 aq) using MeCN/H₂O (37/63) to provide rac-1,3-dimethyl-N-(2-(1-methylpyrrolidin-2-yl)-1H-pyrrolo[3,2-c]pyridin-6-yl)-1H-indazole-6-carboxamide (Example 75, 4.6 g, 60%) as a light yellow solid. Mass spec: m/z 389 [M+H]⁺; ¹H NMR (300 MHz, MeOH-d₄) δ ppm 8.93 (s, 1H), 8.28 (s, 1H), 7.89 (t, J=8.7 Hz, 2H), 7.79 (d, J=8.7 Hz, 1H), 7.24 (s, 1H), 4.81-4.83 (m, 1H), 4.10 (s, 3H), 3.80 (br, 1H), 3.31-3.44 (m, 1H), 2.95 (br, 3H), 2.62-2.76 (m, 1H), 2.49-2.58 (m, 4H), 2.30-2.41 (m, 2H).

### Step 3

### Example 76: (R)-1,3-dimethyl-N-(2-(1-methylpyrrolidin-2-yl)-1H-pyrrolo[3,2-c]pyridin-6-yl)-1H-indazole-6-carboxamide and Example 77: (S)-1,3-dimethyl-N-(2-(1-methylpyrrolidin-2-yl)-1H-pyrrolo[3,2-c]pyridin-6-yl)-1H-indazole-6-carboxamide

Rac-1,3-dimethyl-N-(2-(1-methylpyrrolidin-2-yl)-1H-pyrrolo[3,2-c]pyridin-6-yl)-1H-indazole-6-carboxamide (Intermediate 328, 5.46 g, 13.4 mmol) was separated into the enantiomeric Example 76 and Example 77 using the following conditions: column: Chiral Art Amylose-C 30×250 mm, 5.0 μm; Eluent: CO₂/MeOH (1% of 2M solution NH₃ in MeOH) 1/1; Flow rate: 85 mL/min to provide:

### Example 76: (R)-1,3-dimethyl-N-(2-(1-methylpyrrolidin-2-yl)-1H-pyrrolo[3,2-c]pyridin-6-yl)-1H-indazole-6-carboxamide

First eluting compound (retention time=7.39 min)

¹H NMR (400 MHz, MeOH-d₄) δ ppm 8.51 (s, 1H), 8.21 (s, 1H), 8.13 (s, 1H), 7.80 (d, J=8.4 Hz, 1H), 7.71 (d, J=7.2 Hz, 1H), 6.50 (s, 1H), 4.06 (s, 3H), 3.42 (t, J=8.4 Hz, 1H), 3.19-3.24 (m, 1H), 2.56 (s, 3H), 2.31-2.41 (m, 1H), 2.24-2.29 (m, 4H), 1.94-2.05 (m, 2H), 1.86-1.93 (m, 1H). Mass spec: m/z: 389 [M+H]⁺.

### Example 77: (S)-1,3-dimethyl-N-(2-(1-methylpyrrolidin-2-yl)-1H-pyrrolo[3,2-c]pyridin-6-yl)-1H-indazole-6-carboxamide

Second eluting compound (retention time=12.1 min)

¹H NMR (400 MHz, MeOH-d₄) δ ppm 8.53 (s, 1H), 8.17-8.21 (m, 2H), 7.71-7.85 (m, 2H), 6.53 (s, 1H), 4.08 (s, 3H), 3.45 (t, J=8.00 Hz, 1H), 3.22 (t, J=7.2 Hz, 1H), 2.58 (s, 3H), 2.26-2.44 (m, 5H), 2.01-2.09 (m, 3H). Mass spec: m/z: 389 [M+H]⁺.

### Example 76 was Also Prepared Following Scheme 71

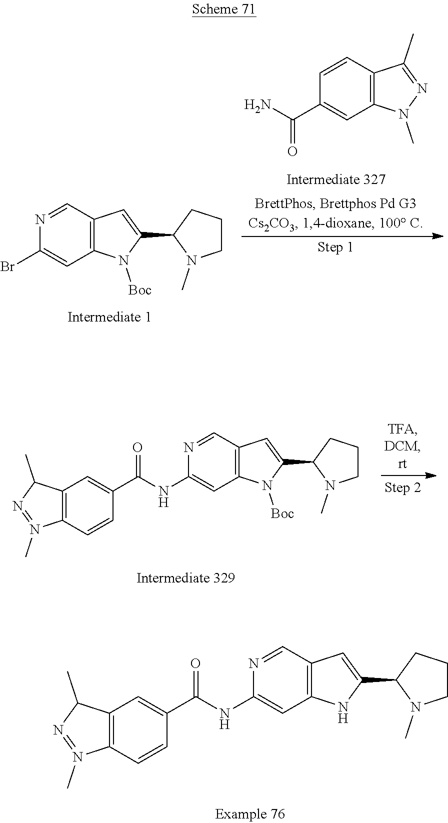

### Step 1

### Intermediate 329: tert-butyl (R)-6-(1,3-dimethyl-1H-indazole-6-carboxamido)-2-(1-methylpyrrolidin-2-yl)-1H-pyrrolo[3,2-c]pyridine-1-carboxylate

A 50 mL round-bottom flask was charged with tert-butyl (R)-6-bromo-2-(1-methylpyrrolidin-2-yl)-1H-pyrrolo[3,2-c]pyridine-1-carboxylate (Intermediate 1, 400 mg, 0.999 mmol), 1,3-dimethyl-1H-indazole-6-carboxamide (Intermediate 327, 227 mg, 1.20 mmol), Brettphos Pd G3 (453 mg, 0.499 mmol), Brettphos (322 mg, 0.599 mmol), Cs₂CO₃ (1.63 g, 5.00 mmol), 1,4-dioxane (10 mL) under nitrogen. The reaction was stirred for 15 h at 100° C. under nitrogen and concentrated under reduced pressure. The residue was chromatographed on a silica gel column with MeOH/DCM (4/96) to provide tert-butyl (R)-6-(1,3-dimethyl-1H-indazole-6-carboxamido)-2-(1-methylpyrrolidin-2-yl)-1H-pyrrolo[3,2-c]pyridine-1-carboxylate (Intermediate 329, 350 mg, 68.1%) as a yellow solid. Mass spec: m/z: 489.1 [M+H]⁺.

### Step 2

### Example 76: (R)-1,3-dimethyl-N-(2-(1-methylpyrrolidin-2-yl)-1H-pyrrolo[3,2-c]pyridin-6-yl)-1H-indazole-6-carboxamide

A 100 mL round-bottom flask was charged with tert-butyl (R)-6-(1,3-dimethyl-1H-indazole-6-carboxamido)-2-(1-methylpyrrolidin-2-yl)-1H-pyrrolo[3,2-c]pyridine-1-carboxylate (Intermediate 329, 350 mg, 0.645 mmol), DCM (10 mL) and TFA (3 mL) and the reaction was stirred for 2 h at room temperature and then concentrated under reduced pressure to provide crude. The crude was chromatographed on a silica gel column with MeCN/water (50/50) to provide (R)-1,3-dimethyl-N-(2-(1-methylpyrrolidin-2-yl)-1H-pyrrolo[3,2-c]pyridin-6-yl)-1H-indazole-6-carboxamide (148.9 mg, 59.33%) as a off-white solid. ¹H NMR (400 MHz, MeOH-d₄) δ ppm 8.51 (s, 1H), 8.21 (s, 1H), 8.13 (s, 1H), 7.80 (d, J=8.4 Hz, 1H), 7.71 (d, J=7.2 Hz, 1H), 6.50 (s, 1H), 4.06 (s, 3H), 3.42 (t, J=8.4 Hz, 1H), 3.19-3.24 (m, 1H), 2.56 (s, 3H), 2.31-2.41 (m, 1H), 2.24-2.29 (m, 4H), 1.94-2.05 (m, 2H), 1.86-1.93 (m, 1H). Mass spec: m/z: 389 [M+H]⁺.

### Intermediate 327 was Synthesised Following Scheme 72

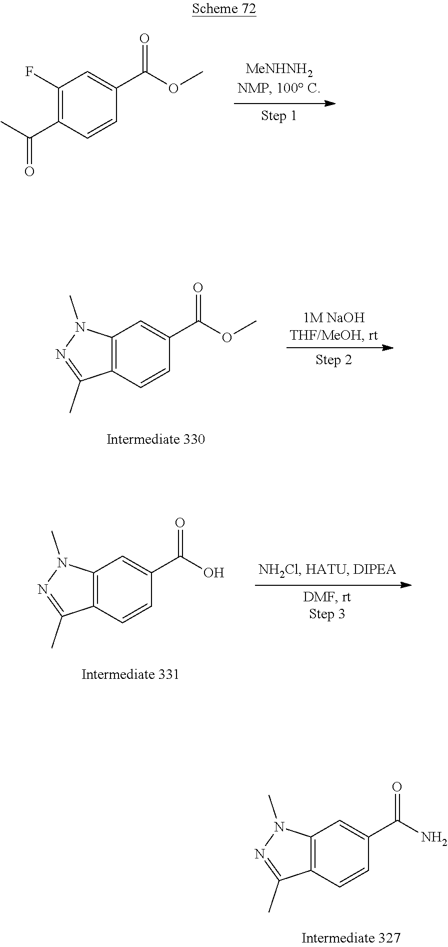

### Step 1

### Intermediate 330: Methyl 1,3-dimethyl-1H-indazole-6-carboxylate

To a solution of methyl 4-acetyl-3-fluorobenzoate (CAS: 1059549-72-2, 5.00 g, 24.2 mmol) in NMP (35 mL) was added methylhydrazine (1.5 mL, 29.1 mmol) and the mixture was stirred overnight at 100° C. in a sealed tube. The solution was cooled to rt, diluted with H₂O (35 mL) and the aqueous layer was extracted with EtOAc (40 mL). The organic layer was washed three times with H₂O, dried over anhydrous MgSO₄, filtered, and concentrated to dryness to afford methyl 1,3-dimethyl-1H-indazole-6-carboxylate (Intermediate 330: 4.3 g, 87%) as an orange oil which was used in Step 2 without further purification. Mass spec: m/z: 205 [M+H]⁺; H NMR (400 MHz, DMSO-d₆) δ ppm 8.21 (s, 1H), 7.81 (d, J=8.5 Hz, 1H), 7.65 (dd, J=8.5, 1.3 Hz, 1H), 4.03 (s, 3H), 3.90 (s, 3H), 2.50 (s, 3H).

### Step 2

### Intermediate 331: 1,3-dimethyl-1H-indazole-6-carboxylic acid

To a solution of methyl 1,3-dimethylindazole-6-carboxylate (Intermediate 330: 4.3 g, 20.0 mmol) in a mixture of THF (50 mL)/MeOH (50 mL) was added a NaOH solution (60 mL, 60.0 mmol, 1M in water) dropwise over 14 min. The resulting mixture was stirred at rt for 45 min. The solvents were removed under reduced pressure and the residue was acidified with a solution of HCl (60 mL, 1N in H₂O). The precipitate was filtered, rinsed with H₂O (20 mL) and dried under reduced pressure at 40° C. overnight to afford 1,3-dimethyl-1H-indazole-6-carboxylic acid (Intermediate 331: 3.85 g, quant.) as a yellow solid. Mass spec: m/z: 191 [M+H]⁺; ¹H NMR (400 MHz, DMSO-d₆) δ ppm 13.04 (s, 1H), 8.19 (t, J=1.1 Hz, 1H), 7.79 (dd, J=8.3, 0.8 Hz, 1H), 7.65 (dd, J=8.4, 1.3 Hz, 1H), 4.03 (s, 3H), 2.51 (s, 3H).

### Step 3

### Intermediate 327: 1,3-dimethyl-1H-indazole-6-carboxamide

A 50 mL round-bottom flask was charged with 1,3-dimethylindazole-6-carboxylic acid (Intermediate 331, 200 mg, 1.05 mmol), NH₄Cl (281 mg, 5.26 mmol), HATU (600 mg, 1.58 mmol), DIPEA (408 mg, 3.16 mmol) in DMF (5 mL) and the reaction was stirred overnight at room temperature. The volatiles were removed under reduced pressure and the mixture was chromatographed on a C18 silica column, eluting with MeCN/water (with 0.5% TFA) (87/13) to provide 1,3-dimethyl-1H-indazole-6-carboxamide (Intermediate 327, 133 mg, 60%) as a white solid. ¹H NMR (400 MHz, DMSO-d₆) δ ppm 8.11 (t, J=1.1 Hz, 1H), 8.02 (s, 1H), 7.74 (dd, J=8.4, 0.8 Hz, 1H), 7.60 (dd, J=8.4, 1.4 Hz, 1H), 7.41 (s, 1H), 4.00 (s, 3H), 2.49 (s, 3H). Mass spec: m/z: 190.1[M+H]⁺.

### Examples 78 and 79 were Synthesized Following Scheme 73

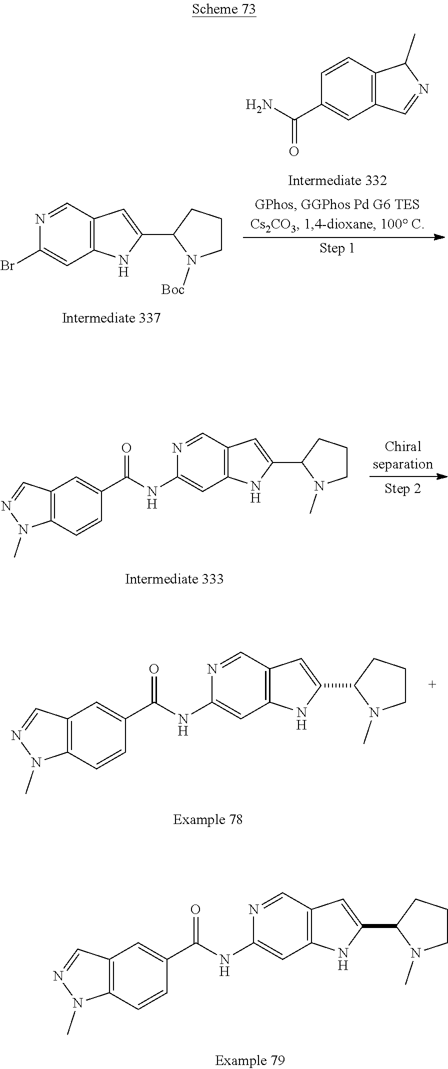

### Step 1

### Intermediate 333: rac-1-methyl-N-(2-(1-methylpyrrolidin-2-yl)-1H-pyrrolo[3,2-c]pyridin-6-yl)-1H-indazole-5-carboxamide

To a solution of rac-2-{6-bromo-1H-pyrrolo[3,2-c]pyridin-2-yl}-1-methylpyrrolidine (Intermediate 15, 100 mg, 0.321 mmol) in 1,4-dioxane (2 mL) were added 1-methylindazole-5-carboxamide (Intermediate 332, 75.0 mg, 0.385 mmol), Cs₂CO₃ (314 mg, 0.963 mmol), [3-(tert-butoxy)-6-methoxy-2′,6′-bis(propan-2-yl)-[1,1′-biphenyl]-2-yl]dicyclohexylphosphane Gphos (17.0 mg, 0.032 mmol) and Pd GPhos G6 TES (30.0 mg, 0.032 mmol). The reaction mixture was stirred at 100° C. for 3 h under nitrogen atmosphere and quenched with water. The resulting mixture was concentrated under reduced pressure and the crude material was chromatographed by reverse-phase flash chromatography (C18 aq) using MeCN/H₂O (75/25) to provide rac-1-methyl-N-(2-(1-methylpyrrolidin-2-yl)-1H-pyrrolo[3,2-c]pyridin-6-yl)-1H-indazole-5-carboxamide (Intermediate 333, 50 mg 37%) as a brown oil. Mass spec: m/z 375 [M+H]⁺.

### Step 2

### Example 78: 1-methyl-N-{2-[(rel-2S)-1-methylpyrrolidin-2-yl]-1H-pyrrolo[3,2-c]pyridin-6-yl}indazole-5-carboxamide and Example 79: 1-methyl-N-{2-[(rel-2R)-1-methylpyrrolidin-2-yl]-1H-pyrrolo[3,2-c]pyridin-6-yl}indazole-5-carboxamide

Rac-1-methyl-N-(2-(1-methylpyrrolidin-2-yl)-1H-pyrrolo[3,2-c]pyridin-6-yl)-1H-indazole-5-carboxamide (Intermediate 333, 50 mg) was separated into the enantiomeric Examples 78 and 79 using the following conditions: column: Chiral Art Cellulose-SB 30×250 mm, 5.0 um; Eluent: CO2/IPA (1% of 2M solution NH₃ in MeOH) 6/4; Flow rate: 85 mL/min to provide:

### Example 79: 1-methyl-N-{2-[(rel-2S)-1-methylpyrrolidin-2-yl]-1H-pyrrolo[3,2-c]pyridin-6-yl}indazole-5-carboxamide

First eluting compound (retention time=5.33 min)

Mass spec: m/z: 375 [M+H]f; ¹H NMR (400 MHz, DMSO-d₆) δ ppm 11.36 (s, 1H), 10.45 (s, 1H), 8.51-8.56 (m, 2H), 8.21 (s, 2H), 8.08-8.10 (m, 1H), 7.72-7.74 (m, 1H), 6.40 (s, 1H), 4.10 (s, 3H), 3.33 (s, 1H), 3.10-3.19 (m, 1H), 2.26-2.28 (m, 1H), 2.15-2.17 (m, 4H), 1.88-1.90 (m, 3H).

### Example 79: 1-methyl-N-{2-[(rel-2R)-1-methylpyrrolidin-2-yl]-1H-pyrrolo[3,2-c]pyridin-6-yl}indazole-5-carboxamide

Second eluting compound (retention time=6.42 min)

Mass spec: m/z: 375 [M+H]f; ¹H NMR (400 MHz, DMSO-d₆) δ ppm 11.36 (s, 1H), 10.45 (s, 1H), 8.51-8.56 (m, 2H), 8.21 (s, 2H), 8.08-8.11 (m, 1H), 7.72-7.74 (m, 1H), 6.40 (s, 1H), 4.10 (s, 3H), 3.33 (s, 1H), 3.10-3.19 (m, 1H), 2.26-2.30 (m, 1H), 2.09-2.20 (m, 4H), 1.75-2.01 (m, 3H).

### Intermediate 332: 1-methyl-1H-indazole-5-carboxamide

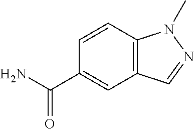

A stirred solution of 1-methyl-1H-indazole-5-carboxylic acid (CAS No: 1176754-31-6, 2.50 g, 14.2 mmol) in anhydrous DCM (125 mL), with a few drops of DMF, was cooled to 0° C. before addition of thionyl chloride (1.1 mL, 15.6 mmol). The reaction mixture was stirred 4 h at rt until consumption of starting material observed. The mixture was concentrated to dryness and re-dissolved in DCM (110 mL). A solution of NH₄OH (11 mL, 71.0 mmol, 25% in water) was added and the mixture was stirred vigorously overnight at rt. Water was added and the mixture was extracted three times with EtOAc. The combined organic layers were dried over a phase separator and concentrated to dryness. The residue was taken up in DCM and the precipitate was filtered, washed with DCM and dried under vacuum at 40° C. to afford 1-methyl-1H-indazole-5-carboxamide (Intermediate 332: 1.6 g, 64%) as a beige solid. ¹H NMR (400 MHz, DMSO-d₆) δ ppm 8.35 (dd, J=1.5, 0.7 Hz, 1H), 8.17 (d, J=0.9 Hz, 1H), 7.98 (s, 1H), 7.93 (dd, J=8.8, 1.6 Hz, 1H), 7.67 (d, J=8.8 Hz, 1H), 7.27 (s, 1H), 4.07 (s, 3H). Mass spec: m/z: 176 [M+H]⁺.

### Example 80: N-{3-chloro-2-[(2R)-1-methylpyrrolidin-2-yl]-1H-pyrrolo[3,2-c]pyridin-6-yl}-1,3-dimethylindazole-6-carboxamide

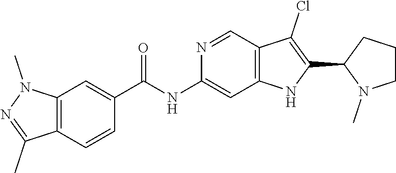 [SMILES: Cc1nn(C)c2cc(C(=O)Nc3cc4[nH]c([C@H]5CCCN5C)c(Cl)c4cn3)ccc12]

To a stirred solution of 1,3-dimethyl-N-{2-[(2R)-1-methylpyrrolidin-2-yl]-1H-pyrrolo[3,2-c]pyridin-6-yl}indazole-6-carboxamide (Example 76, 90 mg, 0.232 mmol) in DMF (2 mL) was added NCS (46 mg, 0.348 mmol) dropwise at 0° C. The reaction mixture was stirred overnight at rt, then concentrated under reduced pressure. The residue was purified by preparative HPLC (Column: XBridge OBD, 30×150 mm, 5 μm; Eluent: MeCN in Water (with 10 mmol/L NH₄HCO₃) from 34 to 64%; flow rate: 60 mL/min) to afford N-{3-chloro-2-[(2R)-1-methylpyrrolidin-2-yl]-1H-pyrrolo[3,2-c]pyridin-6-yl}-1,3-dimethylindazole-6-carboxamide (Example 80, 52.8 mg, 55%) as an off-white solid. ¹H NMR (400 MHz, MeOH-d₄) δ ppm 8.52 (s, 1H), 8.28 (s, 1H), 8.17 (s, 1H), 7.83-7.85 (m, 1H), 7.72-7.74 (m, 1H), 4.09 (s, 3H), 3.67-3.73 (m, 1H), 3.22-3.26 (m, 1H), 2.64 (s, 3H), 2.42-2.47 (m, 1H), 2.27 (s, 4H), 1.92-2.12 (m, 3H). Mass spec: m/z: 423 [M+H]⁺.

### Example 81: N-{3-chloro-2-[(2S)-1-methylpyrrolidin-2-yl]-1H-pyrrolo[3,2-c]pyridin-6-yl}-1,3-dimethylindazole-6-carboxamide

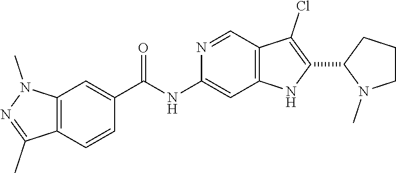 [SMILES: Cc1nn(C)c2cc(C(=O)Nc3cc4[nH]c([C@@H]5CCCN5C)c(Cl)c4cn3)ccc12]

Following an analogous procedure to Example 80, using 1,3-dimethyl-N-{2-[(2S)-1-methylpyrrolidin-2-yl]-1H-pyrrolo[3,2-c]pyridin-6-yl}indazole-6-carboxamide (Example 77, 200 mg, 0.515 mmol), afforded N-{3-chloro-2-[(2S)-1-methylpyrrolidin-2-yl]-1H-pyrrolo[3,2-c]pyridin-6-yl}-1,3-dimethylindazole-6-carboxamide (Example 81: 64.8 mg, 31%) as an off-white solid. ¹H NMR (400 MHz, MeOH-d₄) δ ppm 8.50 (s, 1H), 8.30 (s, 1H), 8.16 (s, 1H), 7.82-7.84 (m, 1H), 7.71-7.77 (m, 1H), 4.08 (s, 3H), 3.68-3.80 (m, 1H), 3.22-3.26 (m, 1H), 2.58 (s, 3H), 2.38-2.47 (m, 1H), 2.27 (s, 4H), 1.92-2.11 (m, 3H). Mass spec: m/z: 423 [M+H]⁺.

### Example 82: (R)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide

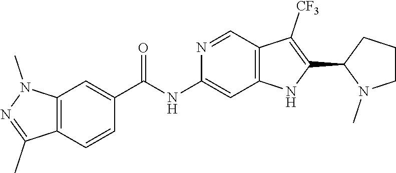 [SMILES: Cc1nn(C)c2cc(C(=O)Nc3cc4[nH]c([C@H]5CCCN5C)c(C(F)(F)F)c4cn3)ccc12]

To a solution of(R)-1,3-dimethyl-N-(2-(1-methylpyrrolidin-2-yl)-1H-pyrrolo[3,2-c]pyridin-6-yl)-1H-indazole-6-carboxamide (Example 76, 400 mg, 0.978 mmol) in MeCN (10 mL) was added K₂CO₃ (270 mg, 1.96 mmol) and 2,8-difluoro-5-(trifluoromethyl)-5H-dibenzo[b,d]thiophen-5-ium trifluoromethanesulfonate (643 mg, 1.47 mmol) and the mixture was stirred at 80° C. overnight. Water (1 mL) was added and the mixture chromatographed on a C18 column, eluting with water/MeCN (25/75) to provide crude product. The crude product was purified by Chiral-HPLC (Column: YMC-Actus Triart C18 ExRS, 30*150 mm, 5 μm; Mobile Phase A: Water (+10 mmol/L NH₄HCO₃+0.05% NH₃·H₂O), Mobile Phase B: MeCN; Flow rate: 60 mL/min Gradient: 5% B to 20% B in 8 min) to provide (R)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide (Example 82, 3.1 mg, 0.7%) as a white solid. 1H NMR (400 MHz, DMSO-d6) δ ppm 12.01 (br, 1H), 10.88 (br, 1H), 8.43-8.52 (m, 2H), 7.73-7.82 (m, 2H), 6.53-6.66 (m, 1H), 4.07 (s, 3H), 3.45-3.53 (m, 1H), 3.15-3.25 (m, 1H), 2.55 (s, 3H), 2.35-2.40 (m, 2H), 2.12-2.26 (m, 3H), 1.70-2.05 (m, 3H). Mass spec: m/z: 457.1 [M+H]⁺.

### Example 83: (S)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide

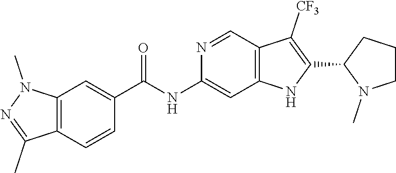 [SMILES: Cc1nn(C)c2cc(C(=O)Nc3cc4[nH]c([C@@H]5CCCN5C)c(C(F)(F)F)c4cn3)ccc12]

Example 83 was synthesised in an analogous manner to Example 82, using (S)-1,3-dimethyl-N-(2-(1-methylpyrrolidin-2-yl)-1H-pyrrolo[3,2-c]pyridin-6-yl)-1H-indazole-6-carboxamide (Example 77) as the amide and 2,8-difluoro-5-(trifluoromethyl)-5H-dibenzo[b,d]thiophen-5-ium trifluoromethanesulfonate (CAS No: 1961266-44-3), to afford (S)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide (Example 83, 15 mg, 3%) as a white solid. ¹H NMR (400 MHz, DMSO-d₆) δ ppm 12.15 (s, 1H), 10.76 (s, 1H), 8.62 (s, 1H), 8.43 (d, J=14 Hz, 2H), 7.74-7.82 (m, 2H), 4.06 (s, 3H), 3.62-3.65 (m, 1H), 3.20-3.24 (m, 1H), 2.50 (s, 3H), 2.30-2.35 (m, 1H), 2.20-2.24 (m, 4H), 1.90-2.00 (m, 1H), 1.70-1.90 (m, 2H). Mass spec: m/z: 457 [M+H]⁺.

### Examples 84 and 85 were Synthesized Following Scheme 74

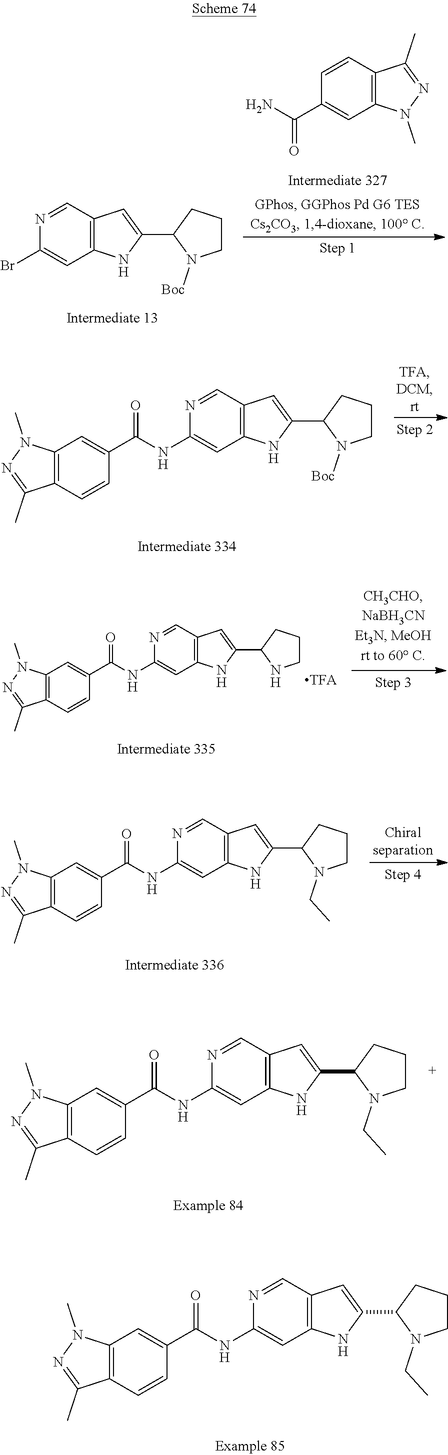

### Step 1

### Intermediate 334: rac-tert-butyl 2-[6-(1,3-dimethylindazole-6-amido)-1H-pyrrolo[3,2-c]pyridin-2-yl]pyrrolidine-1-carboxylate

To a solution of rac-tert-butyl 2-{6-bromo-1H-pyrrolo[3,2-c]pyridin-2-yl}pyrrolidine-1-carboxylate (Intermediate 13: 700 mg, 1.91 mmol), 1,3-dimethylindazole-6-carboxamide (Intermediate 327, 434 mg, 2.29 mmol), Cs₂CO₃ (1.25 g, 3.82 mmol) in 1,4-dioxane (20 mL) were added Gphos (103 mg, 0.191 mmol) and Gphos Pd G6 TES (180 mg, 0.191 mmol). The reaction mixture was stirred overnight at 100° C. under nitrogen atmosphere and quenched with water (80 mL). The resulting solution was extracted three times with EtOAc and the combined organic layers were washed with brine, dried over anhydrous Na₂SO₄, filtered, and concentrated under reduced pressure. The crude material was purified by flash chromatography on silica gel using DCM/MeOH (95/5) as eluent to afford rac-tert-butyl 2-[6-(1,3-dimethylindazole-6-amido)-1H-pyrrolo[3,2-c]pyridin-2-yl]pyrrolidine-1-carboxylate (Intermediate 334: 700 mg, 80%) as a yellow oil. Mass spec: m/z: 475 [M+H]⁺.

### Step 2

### Intermediate 335: 1,3-dimethyl-N-[2-(pyrrolidin-2-yl)-1H-pyrrolo[3,2-c]pyridin-6-yl]indazole-6-carboxamide trifluoroacetate

To a solution of rac-tert-butyl 2-[6-(1,3-dimethylindazole-6-amido)-1H-pyrrolo[3,2-c]pyridin-2-yl]pyrrolidine-1-carboxylate (Intermediate 334: 700 mg, 1.48 mmol) in DCM (30 mL) was added TFA (10 mL). The reaction mixture was stirred for 2 h at rt and concentrated under reduced pressure to provide rac-1,3-dimethyl-N-[2-(pyrrolidin-2-yl)-1H-pyrrolo[3,2-c]pyridin-6-yl]indazole-6-carboxamide trifluoroacetate (Intermediate 335: 600 mg) as a brown oil which was used in the next step without further purification. Mass spec: m/z: 375 [M+H]⁺.

### Step 3

### Intermediate 336: rac-N-[2-(1-ethylpyrrolidin-2-yl)-1H-pyrrolo[3,2-c]pyridin-6-yl]-1,3-dimethylindazole-6-carboxamide

To a solution of rac-1,3-dimethyl-N-[2-(pyrrolidin-2-yl)-1H-pyrrolo[3,2-c]pyridin-6-yl]indazole-6-carboxamide (Intermediate 335: 120 mg, 0.320 mmol) in MeOH (4 mL) was added acetaldehyde (0.32 mL, 1.60 mmol, 5M in THF) and Et₃N (97.3 mg, 0.960 mmol). The reaction mixture was stirred for 1 h at rt. Then, NaBH₃CN (48.5 mg, 0.771 mmol) was added and the reaction mixture was stirred for 1 h at 60° C. The mixture was concentrated under reduced pressure and the crude material was chromatographed by reverse-phase flash chromatography (C18 aq) using MeCN/H₂O (35/65) to provide rac-N-[2-(1-ethylpyrrolidin-2-yl)-1H-pyrrolo[3,2-c]pyridin-6-yl]-1,3-dimethylindazole-6-carboxamide (Intermediate 336, 60 mg, 36%) as a brown oil. Mass spec: m/z: 403 [M+H]⁺.

### Step 4

### Example 84: N-{2-[(rel-2R)-1-ethylpyrrolidin-2-yl]-1H-pyrrolo[3,2-c]pyridin-6-yl}-1,3-dimethylindazole-6-carboxamide and Example 85: N-{2-[(rel-2S)-1-ethylpyrrolidin-2-yl]-1H-pyrrolo[3,2-c]pyridin-6-yl}-1,3-dimethylindazole-6-carboxamide

Rac-N-[2-(1-ethylpyrrolidin-2-yl)-1H-pyrrolo[3,2-c]pyridin-6-yl]-1,3-dimethylindazole-6-carboxamide (Intermediate 336, 60 mg) was separated into the enantiomeric Example 84 and Example 85 using the following conditions: column: ChiralPak IF 2×25 cm, 5.0 um; Eluent: MTBE (0.5% of 2M solution NH₃ in MeOH)/EtOH 95/5; Flow rate: 20 mL/min to provide:

### Example 84: N-{2-[(rel-2R)-1-ethylpyrrolidin-2-yl]-1H-pyrrolo[3,2-c]pyridin-6-yl}-1,3-dimethylindazole-6-carboxamide

First eluting compound (retention time=11.8 min)

Mass spec: m/z: 403 [M+H]; ¹H NMR (400 MHz, MeOH-d₄) δ ppm 8.57 (s, 1H), 8.22-8.25 (m, 2H), 7.87-7.88 (m, 1H), 7.79-7.80 (m, 1H), 6.54 (s, 1H), 4.13 (s, 3H), 3.60-3.62 (m, 1H), 3.39 (s, 1H), 2.77-2.79 (m, 1H), 2.63 (s, 3H), 2.33-2.38 (m, 3H), 1.99-2.11 (m, 3H), 1.05-1.19 (m, 3H).

### Example 85: N-{2-[(rel-2S)-1-ethylpyrrolidin-2-yl]-1H-pyrrolo[3,2-c]pyridin-6-yl}-1,3-dimethylindazole-6-carboxamide

Second eluting compound (retention time=16.7 min)

Mass spec: m/z: 403 [M+H]; ¹H NMR (400 MHz, MeOH-d₄) δ ppm 8.52 (s, 1H), 8.17-8.20 (m, 2H), 7.83-7.85 (m, 1H), 7.72-7.74 (m, 1H), 6.50 (s, 1H), 4.09 (s, 3H), 3.60-3.62 (m, 1H), 3.39 (s, 1H), 2.75-2.77 (m, 1H), 2.58 (s, 3H), 2.33-2.38 (m, 3H), 1.99-2.11 (m, 3H), 1.05-1.19 (m, 3H).

### Examples 86 and 87 were Synthesized Following Scheme 75

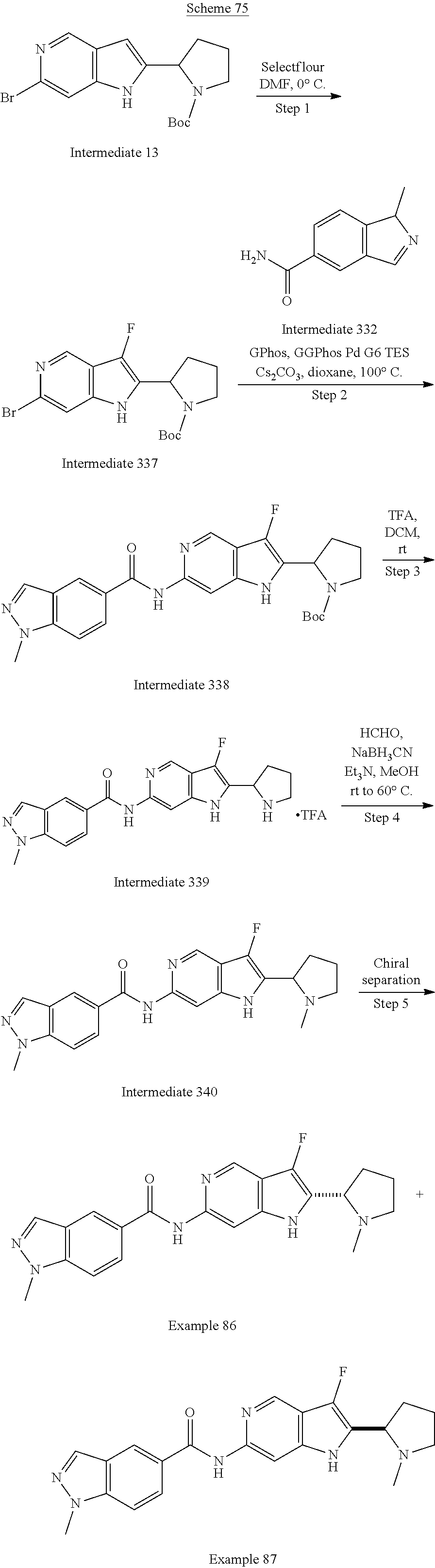

### Step 1

### Intermediate 337: rac-tert-butyl 2-{6-bromo-3-fluoro-1H-pyrrolo[3,2-c]pyridin-2-yl}pyrrolidine-1-carboxylate

To a solution of rac-tert-butyl 2-{6-bromo-1H-pyrrolo[3,2-c]pyridin-2-yl}pyrrolidine-1-carboxylate (Intermediate 13: 6.40 g, 0.015 mol) in DMF (4 mL) was added selectfluor (5.55 g, 0.015 mol). The reaction mixture was stirred at 0° C. for 3 h and quenched with water. The mixture was concentrated under reduced pressure and the residue was chromatographed by reverse-phase flash chromatography (C18 silica) using MeCN/H₂O (55/45) to provide rac-tert-butyl 2-{6-bromo-3-fluoro-1H-pyrrolo[3,2-c]pyridin-2-yl}pyrrolidine-1-carboxylate (Intermediate 337: 800 mg, 12%) as a yellow oil. Mass spec: m/z 384 [M+H]⁺.

### Step 2

### Intermediate 338: rac-tert-butyl 2-[3-fluoro-6-(1-methylindazole-5-amido)-1H-pyrrolo[3,2-c]pyridin-2-yl]pyrrolidine-1-carboxylate

To a solution of rac-tert-butyl 2-{6-bromo-3-fluoro-1H-pyrrolo[3,2-c]pyridin-2-yl}pyrrolidine-1-carboxylate (Intermediate 337: 750 mg, 1.75 mmol) in 1,4-dioxane (8 mL) were added 1-methylindazole-5-carboxamide (Intermediate 332: 410 mg, 2.11 mmol), Cs₂CO₃ (1.71 g, 5.25 mmol), GPhos (94.0 mg, 0.175 mmol) and GPhos Pd G6 TES (166 mg, 0.175 mmol) under nitrogen atmosphere. The reaction mixture was stirred at 100° C. for 3 h and then quenched with water. The resulting solution was extracted three times with EtOAc and the combined organic layers were washed with brine, dried over anhydrous Na₂SO₄, filtered, and concentrated under reduced pressure. The crude material was purified by flash chromatography on silica gel using DCM/MeOH (96/4) as eluent to afford rac-tert-butyl 2-[3-fluoro-6-(1-methylindazole-5-amido)-1H-pyrrolo[3,2-c]pyridin-2-yl]pyrrolidine-1-carboxylate (Intermediate 338: 350 mg, 37%) as a yellow solid. Mass spec: m/z 479 [M+H]⁺.

### Step 3

### Intermediate 339: rac-N-[3-fluoro-2-(pyrrolidin-2-yl)-1H-pyrrolo[3,2-c]pyridin-6-yl]-1-methylindazole-5-carboxamide trifluoroacetate

To a solution of rac-tert-butyl 2-[3-fluoro-6-(1-methylindazole-5-amido)-1H-pyrrolo[3,2-c]pyridin-2-yl]pyrrolidine-1-carboxylate (Intermediate 338: 350 mg, 0.811 mmol) in DCM (3 mL) was added TFA (1 mL). The reaction mixture was stirred at rt for 2 h and concentrated under reduced pressure to provide rac-N-[3-fluoro-2-(pyrrolidin-2-yl)-1H-pyrrolo[3,2-c]pyridin-6-yl]-1-methylindazole-5-carboxamide trifluoroacetate (Intermediate 339: 250 mg, 90%) as a yellow oil which was used in Step 4 without further purification. Mass spec: m/z 379 [M+H]⁺.

### Step 4

### Intermediate 340: rac-N-(3-fluoro-2-(1-methylpyrrolidin-2-yl)-1H-pyrrolo[3,2-c]pyridin-6-yl)-1-methyl-1H-indazole-5-carboxamide

To a solution of rac-N-[3-fluoro-2-(pyrrolidin-2-yl)-1H-pyrrolo[3,2-c]pyridin-6-yl]-1-methylindazole-5-carboxamide trifluoroacetate (Intermediate 339: 25 mg, 0.059 mmol) in MeOH (1 mL) were added paraformaldehyde (8.86 mg, 0.295 mmol) and Et₃N (29.9 mg, 0.295 mmol). The reaction mixture was stirred at rt for 30 min prior addition of NaBH₃CN (11.1 mg, 0.177 mmol). The reaction mixture was stirred at 60° C. for 1 h and quenched with water. The mixture was concentrated under reduced pressure and the residue was chromatographed by reverse-phase flash chromatography (C18) using MeCN/H₂O (86/14) to provide rac-N-(3-fluoro-2-(1-methylpyrrolidin-2-yl)-1H-pyrrolo[3,2-c]pyridin-6-yl)-1-methyl-1H-indazole-5-carboxamide (Intermediate 340: 20 mg, 77%) as a white solid. Mass spec: m/z 393 [M+H]⁺.

### Example 86: N-{3-fluoro-2-[(rel-2S)-1-methylpyrrolidin-2-yl]-1H-pyrrolo[3,2-c]pyridin-6-yl}-1-methylindazole-5-carboxamide and Example 87: N-{3-fluoro-2-[(rel-2R)-1-methylpyrrolidin-2-yl]-1H-pyrrolo[3,2-c]pyridin-6-yl}-1-methylindazole-5-carboxamide

Rac-N-(3-fluoro-2-(1-methylpyrrolidin-2-yl)-1H-pyrrolo[3,2-c]pyridin-6-yl)-1-methyl-1H-indazole-5-carboxamide (Intermediate 340, 20 mg) was separated into the enantiomeric Example 86 and Example 87 using the following conditions: column: Chiral Art Cellulose-SC 2×25 cm, 5.0 um; Eluent: (Hexane/DCM 3/1 (0.5% 2M NH₃ in MeOH))/IPA 90/10; Flow rate: 20 mL/min to provide:

### Example 86: N-{3-fluoro-2-[(rel-2S)-1-methylpyrrolidin-2-yl]-1H-pyrrolo[3,2-c]pyridin-6-yl}-1-methylindazole-5-carboxamide

First eluting compound (retention time=9.1 min)

Mass spec: m/z: 393 [M+H]f; ¹H NMR (400 MHz, MeOH-d₄) δ ppm 8.48-8.55 (m, 1H), 8.48 (s, 1H), 8.20-8.21 (m, 1H), 8.18-8.20 (m, 1H), 8.04-8.07 (m, 1H), 7.67-7.70 (m, 1H), 4.12 (s, 3H), 3.58-3.62 (m, 1H), 3.18-3.20 (m, 1H), 2.38-2.43 (m, 1H), 2.23-2.28 (m, 4H), 2.05-2.10 (m, 2H), 1.90-2.00 (m, 1H).

### Example 87: N-{3-fluoro-2-[(rel-2R)-1-methylpyrrolidin-2-yl]-1H-pyrrolo[3,2-c]pyridin-6-yl}-1-methylindazole-5-carboxamide

Second eluting compound (retention time=13.3 min)

Mass spec: m/z: 393 [M+H]f; ¹H NMR (400 MHz, MeOH-d₄) δ ppm 8.56 (s, 1H), 8.48 (s, 1H), 8.20-8.21 (m, 2H), 8.04-8.07 (m, 1H), 7.68-7.70 (m, 1H), 4.12 (s, 3H), 3.59-3.63 (m, 1H), 3.19-3.23 (m, 1H), 2.41-2.43 (m, 1H), 2.22-2.28 (m, 4H), 1.92-2.11 (m, 3H).

### Examples 88 and 89 were Made Following Scheme 76

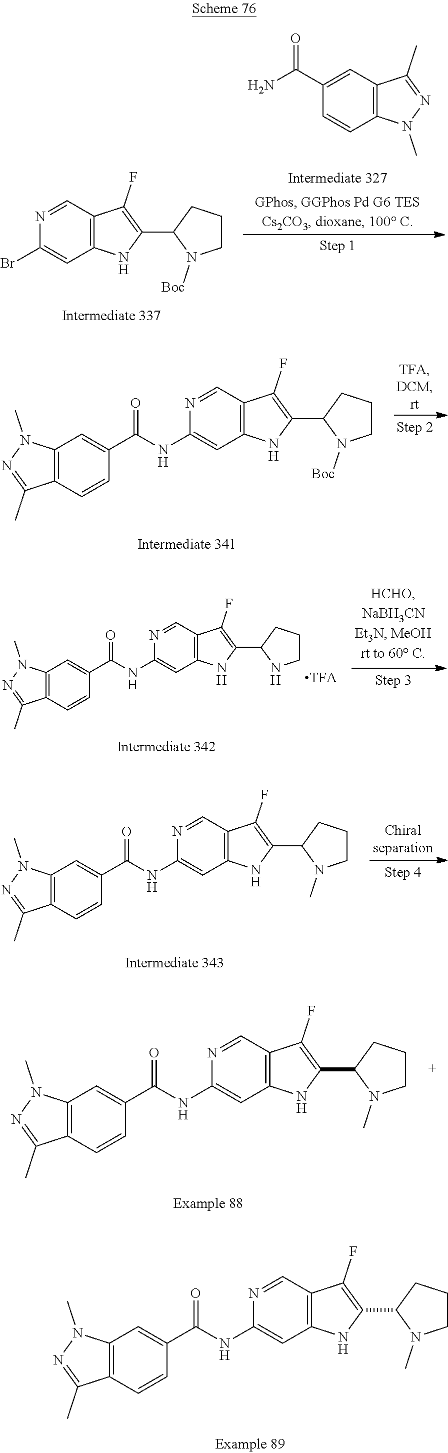

### Step 1

### Intermediate 341: rac-tert-butyl 2-[6-(1,3-dimethylindazole-6-amido)-3-fluoro-1H-pyrrolo[3,2-c]pyridin-2-yl]pyrrolidine-1-carboxylate

Following an analogous procedure to Scheme 75, Step 2, using rac-tert-butyl 2-{6-bromo-3-fluoro-1H-pyrrolo[3,2-c]pyridin-2-yl}pyrrolidine-1-carboxylate as the bromine intermediate (Intermediate 337: 140 mg, 0.328 mmol) and 1,3-dimethylindazole-6-carboxamide as the amine (Intermediate 327: 83 mg, 0.393 mmol), afforded after purification by reverse-phase flash chromatography (C18 aq) using MeCN/H₂O (72/28) rac-tert-butyl 2-[6-(1,3-dimethylindazole-6-amido)-3-fluoro-1H-pyrrolo[3,2-c]pyridin-2-yl]pyrrolidine-1-carboxylate (Intermediate 341: 100 mg, 56%) as a yellow oil. Mass spec: m/z: 493 [M+H]⁺.

### Step 2

### Intermediate 342: rac-N-[3-fluoro-2-(pyrrolidin-2-yl)-1H-pyrrolo[3,2-c]pyridin-6-yl]-1,3-dimethylindazole-6-carboxamide trifluoroacetate

Following an analogous procedure to Scheme 75, Step 3, using rac-tert-butyl 2-[6-(1,3-dimethylindazole-6-amido)-3-fluoro-1H-pyrrolo[3,2-c]pyridin-2-yl]pyrrolidine-1-carboxylate (Intermediate 341: 100 mg, 0.182 mmol), afforded rac-N-[3-fluoro-2-(pyrrolidin-2-yl)-1H-pyrrolo[3,2-c]pyridin-6-yl]-1,3-dimethylindazole-6-carboxamide trifluoroacetate (Intermediate 342: 75 mg, 96%) as a yellow oil. Mass spec: m/z: 393 [M+H]⁺.

### Step 3

### Intermediate 343: rac-N-(3-fluoro-2-(1-methylpyrrolidin-2-yl)-1H-pyrrolo[3,2-c]pyridin-6-yl)-1,3-dimethyl-1H-indazole-6-carboxamide

Following an analogous procedure to Scheme 75, Step 4, using rac-tert-butyl 2-[6-(1,3-dimethylindazole-6-amido)-3-fluoro-1H-pyrrolo[3,2-c]pyridin-2-yl]pyrrolidine-1-carboxylate trifluoroacetate (Intermediate 342: 70 mg, 0.176 mmol), afforded rac-N-(3-fluoro-2-(1-methylpyrrolidin-2-yl)-1H-pyrrolo[3,2-c]pyridin-6-yl)-1,3-dimethyl-1H-indazole-6-carboxamide (Intermediate 343: 50 mg, 65%). Mass spec: m/z: 407 [M+H]⁺.

### Step 4

### Example 88: N-{3-fluoro-2-[(rel-2R)-1-methylpyrrolidin-2-yl]-1H-pyrrolo[3,2-c]pyridin-6-yl}-1,3-dimethylindazole-6-carboxamide and Example 89: N-{3-fluoro-2-[(rel-2S)-1-methylpyrrolidin-2-yl]-1H-pyrrolo[3,2-c]pyridin-6-yl}-1,3-dimethylindazole-6-carboxamide

Rac-N-(3-fluoro-2-(1-methylpyrrolidin-2-yl)-1H-pyrrolo[3,2-c]pyridin-6-yl)-1,3-dimethyl-1H-indazole-6-carboxamide (Intermediate 343, 50 mg) was separated into the enantiomeric Example 88 and Example 89 using the following conditions: column: Chiral Art Amylose-SA 2×25 cm, 5.0 um; Eluent: (Hexane (0.5% 2M NH₃ in MeOH))/EtOH 85/15; Flow rate: 20 mL/min to provide:

### Example 88: N-{3-fluoro-2-[(rel-2R)-1-methylpyrrolidin-2-yl]-1H-pyrrolo[3,2-c]pyridin-6-yl}-1,3-dimethylindazole-6-carboxamide

First eluting compound (retention time=11.3 min)

Mass spec: m/z: 407 [M+H]⁺; ¹H NMR (400 MHz, DMSO-d₆) δ ppm 11.29 (s, 1H), 10.64 (s, 1H), 8.59 (s, 1H), 8.42 (s, 1H), 8.25-8.26 (m, 1H), 7.72-7.81 (m, 2H), 4.05 (s, 3H), 3.33-3.47 (m, 1H), 3.14-3.16 (m, 1H), 2.50-2.51 (m, 3H), 2.24-2.31 (m, 1H), 2.13-2.22 (m, 4H), 1.91-1.95 (m, 3H).

### Example 89: N-{3-fluoro-2-[(rel-2S)-1-methylpyrrolidin-2-yl]-1H-pyrrolo[3,2-c]pyridin-6-yl}-1,3-dimethylindazole-6-carboxamide

Second eluting compound (retention time=14.2 min)

Mass spec: m/z: 407 [M+H]⁺; ¹H NMR (400 MHz, DMSO-d₆) δ ppm 11.29 (s, 1H), 10.64 (s, 1H), 8.59 (s, 1H), 8.42 (s, 1H), 8.25 (s, 1H), 7.72-7.81 (m, 2H), 4.05 (s, 3H), 3.47-3.49 (m, 1H), 3.13-3.16 (m, 1H), 2.50-2.67 (m, 3H), 2.29-2.33 (m, 1H), 2.13-2.27 (m, 4H), 1.82-1.97 (m, 3H).

### Examples 90 and 91 were Synthesized Following Scheme 77

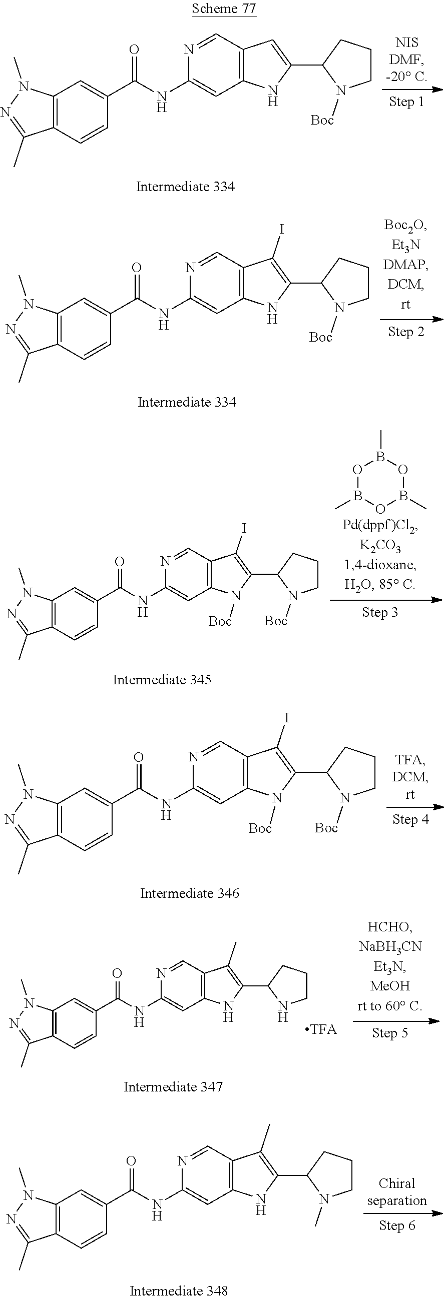

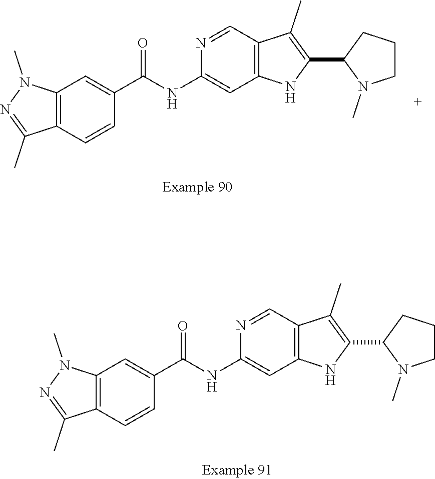

### Step 1

### Intermediate 344: rac-tert-butyl 2-[6-(1,3-dimethylindazole-6-amido)-3-iodo-1H-pyrrolo[3,2-c]pyridin-2-yl]pyrrolidine-1-carboxylate

To a solution of rac-tert-butyl 2-[6-(1,3-dimethylindazole-6-amido)-1H-pyrrolo[3,2-c]pyridin-2-yl]pyrrolidine-1-carboxylate (Intermediate 334, 250 mg, 0.473 mol) in DMF (5 mL) cooled to −20° C. was added NIS (128 mg, 0.567 mmol). The reaction mixture was stirred at −20° C. for 2 h and then quenched with water. The resulting solution was extracted three times with EtOAc and the combined organic layers were washed with brine, dried over anhydrous Na₂SO₄, filtered, and concentrated under reduced pressure. The crude material was purified by flash chromatography on silica gel using petroleum ether/EtOAc (52/48) as eluent to afford rac-tert-butyl 2-[6-(1,3-dimethylindazole-6-amido)-3-iodo-1H-pyrrolo[3,2-c]pyridin-2-yl]pyrrolidine-1-carboxylate (Intermediate 344, 200 mg, 63%) as a yellow oil. Mass spec: m/z 601 [M+H]⁺.

### Step 2

### Intermediate 345: rac-tert-butyl 2-[1-(tert-butoxycarbonyl)-6-[N-(tert-butoxycarbonyl)1,3-dimethylindazole-6-amido]-3-iodopyrrolo[3,2-c]pyridin-2-yl]pyrrolidine-1-carboxylate

To a solution of rac-tert-butyl 2-[6-(1,3-dimethylindazole-6-amido)-3-iodo-1H-pyrrolo[3,2-c]pyridin-2-yl]pyrrolidine-1-carboxylate (Intermediate 344, 200 mg, 0.299 mmol) in DCM (10 mL) were added (Boc)₂O (130 mg, 0.599 mmol), DMAP (10.9 mg, 0.090 mmol) and Et₃N (121 mg, 1.20 mmol). The reaction mixture was stirred at rt for 2 h and then quenched with water. The resulting solution was extracted three times with EtOAc and the combined organic layers were washed with brine, dried over anhydrous Na₂SO₄, filtered, and concentrated under reduced pressure. The crude material was purified by flash chromatography on silica gel using petroleum ether/EtOAc (6/4) as eluent to afford rac-tert-butyl 2-[1-(tert-butoxycarbonyl)-6-[N-(tert-butoxycarbonyl)1,3-dimethylindazole-6-amido]-3-iodopyrrolo[3,2-c]pyridin-2-yl]pyrrolidine-1-carboxylate (Intermediate 345, 200 mg, 77%) as a yellow oil. Mass spec: m/z 801 [M+H]⁺.

### Step 3

### Intermediate 346: rac-tert-butyl 2-(1-(tert-butoxycarbonyl)pyrrolidin-2-yl)-6-(1,3-dimethyl-1H-indazole-6-carboxamido)-3-methyl-1H-pyrrolo[3,2-c]pyridine-1-carboxylate

To a solution of rac-tert-butyl 2-[1-(tert-butoxycarbonyl)-6-[N-(tert-butoxycarbonyl)1,3-dimethylindazole-6-amido]-3-iodopyrrolo[3,2-c]pyridin-2-yl]pyrrolidine-1-carboxylate (Intermediate 345, 200 mg, 0.230 mmol) in 1,4-dioxane (4 mL)/H₂O (1 mL) were added trimethyl-1,3,5,2,4,6-trioxatriborinane (34.5 mg, 0.275 mmol), Pd(dppf)Cl₂ (33.7 mg, 0.046 mmol) and K₂CO₃ (63.5 mg, 0.460 mmol). The reaction mixture was stirred at 80° C. overnight under nitrogen atmosphere and quenched with water. The resulting solution was extracted three times with EtOAc and the combined organic layers were washed with brine, dried over anhydrous Na₂SO₄, filtered, and concentrated under reduced pressure. The crude material was purified by flash chromatography on silica gel using petroleum ether/EtOAc (75/25) as eluent to afford rac-tert-butyl 2-[1-(tert-butoxycarbonyl)-6-[N-(tert-butoxycarbonyl)1,3-dimethylindazole-6-amido]-3-methylpyrrolo[3,2-c]pyridin-2-yl]pyrrolidine-1-carboxylate (Intermediate 346, 100 mg, 51%) as a yellow oil. Mass spec: m/z 689 [M+H]⁺.

### Step 4

### Intermediate 347: rac-1,3-dimethyl-N-[3-methyl-2-(pyrrolidin-2-yl)-1H-pyrrolo[3,2-c]pyridin-6-yl]indazole-6-carboxamide trifluoroacetate

To a solution of rac-tert-butyl 2-[1-(tert-butoxycarbonyl)-6-[N-(tert-butoxycarbonyl)1,3-dimethylindazole-6-amido]-3-methylpyrrolo[3,2-c]pyridin-2-yl]pyrrolidine-1-carboxylate (Intermediate 346, 100 mg, 0.116 mmol) in DCM (3 mL) was added TFA (1 mL). The reaction mixture was stirred at rt for 2 h and concentrated under reduced pressure to afford rac-1,3-dimethyl-N-[3-methyl-2-(pyrrolidin-2-yl)-1H-pyrrolo[3,2-c]pyridin-6-yl]indazole-6-carboxamide trifluoroacetate (Intermediate 347, 55 mg, 89%) as a yellow solid, which was used in Step 5 without further purification. Mass spec: m/z 389 [M+H]⁺.

### Step 5

### Intermediate 348: rac-1,3-dimethyl-N-(3-methyl-2-(1-methylpyrrolidin-2-yl)-1H-pyrrolo[3,2-c]pyridin-6-yl)-1H-indazole-6-carboxamide

To a solution of rac-1,3-dimethyl-N-[3-methyl-2-(pyrrolidin-2-yl)-1H-pyrrolo[3,2-c]pyridin-6-yl]indazole-6-carboxamide trifluoroacetate (Intermediate 347, 40.0 mg, 0.082 mmol) in MeOH (1 mL) were added paraformaldehyde (12.4 mg, 0.411 mmol) and Et₃N (24.9 mg, 0.246 mmol). The reaction mixture was stirred at rt for 30 min prior addition of NaBH₃CN (15.4 mg, 0.246 mmol). The reaction mixture was stirred at 60° C. for 2 h and quenched with water. The mixture was concentrated under reduced pressure and the residue was chromatographed by reverse-phase flash chromatography (C18) using MeCN/H₂O (75/25) to provide rac-1,3-dimethyl-N-(3-methyl-2-(1-methylpyrrolidin-2-yl)-1H-pyrrolo[3,2-c]pyridin-6-yl)-1H-indazole-6-carboxamide (Intermediate 348, 25 mg). Mass spec: m/z 403 [M+H]⁺.

### Step 6

### Example 90: 1,3-dimethyl-N-{3-methyl-2-[(rel-2R)-1-methylpyrrolidin-2-yl]-1H-pyrrolo[3,2-c]pyridin-6-yl}indazole-6-carboxamide and Example 91: 1,3-dimethyl-N-{3-methyl-2-[(rel-2S)-1-methylpyrrolidin-2-yl]-1H-pyrrolo[3,2-c]pyridin-6-yl}indazole-6-carboxamide

Rac-1,3-dimethyl-N-(3-methyl-2-(1-methylpyrrolidin-2-yl)-1H-pyrrolo[3,2-c]pyridin-6-yl)-1H-indazole-6-carboxamide (Intermediate 348, 25 mg) was separated into the enantiomeric Example 90 and Example 91 using the following conditions: column: Chiral Art Amylose-SA 2×25 cm, 5.0 um; Eluent: Hexane (0.5% 2M NH₃ in MeOH)/EtOH 75/25; Flow rate: 20 mL/min to provide:

### Example 90: 1,3-dimethyl-N-{3-methyl-2-[(rel-2R)-1-methylpyrrolidin-2-yl]-1H-pyrrolo[3,2-c]pyridin-6-yl}indazole-6-carboxamide

First eluting compound (retention time=11.3 min)

Mass spec: m/z: 403 [M+H]⁺; ¹H NMR (400 MHz, MeOH-d₄) δ ppm 8.58 (s, 1H), 8.17-8.20 (m, 2H), 7.83-7.85 (m, 1H), 7.71-7.74 (m, 1H), 4.08-4.27 (m, 4H), 3.41-3.55 (m, 1H), 2.78-2.91 (m, 1H), 2.51-2.62 (m, 3H), 2.48-2.50 (m, 3H), 2.35-2.47 (m, 4H), 2.06-2.34 (m, 3H).

### Example 91: 1,3-dimethyl-N-{3-methyl-2-[(rel-2S)-1-methylpyrrolidin-2-yl]-1H-pyrrolo[3,2-c]pyridin-6-yl}indazole-6-carboxamide

Second eluting compound (retention time=14.4 min)

Mass spec: m/z: 403 [M+H]⁺; ¹H NMR (400 MHz, MeOH-d₄) δ ppm 8.64 (s, 1H), 8.18-8.24 (m, 2H), 7.83-7.86 (m, 1H), 7.72-7.74 (m, 1H), 4.53-4.58 (m, 1H), 4.08-4.10 (m, 3H), 3.47-3.71 (m, 1H), 3.02-3.18 (m, 1H), 2.61-2.71 (m, 3H), 2.52-2.60 (m, 3H), 2.31-2.51 (m, 5H), 2.15-2.30 (m, 2H).

### Examples 92 and 93 were Synthesized Following Scheme 78

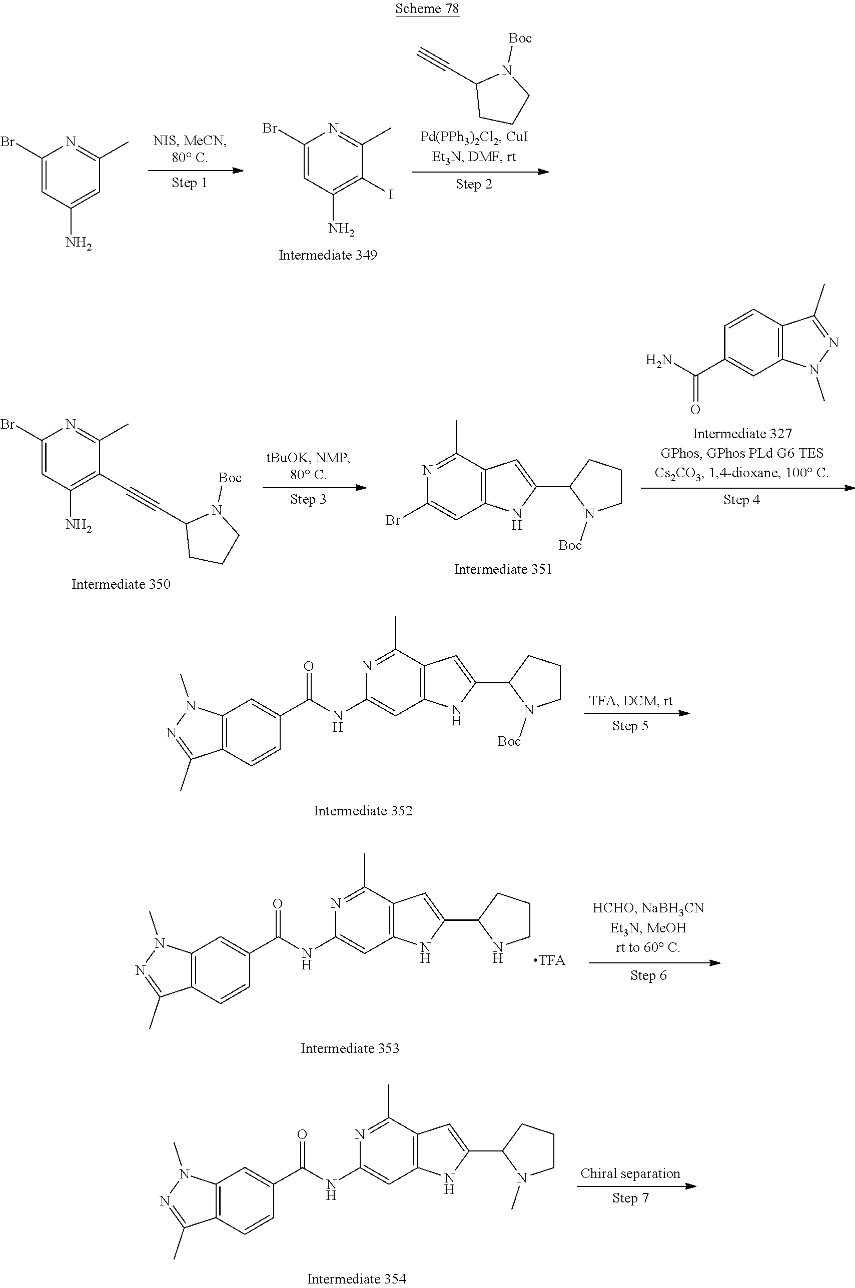

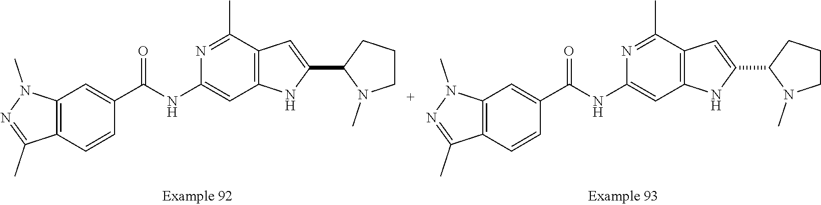

### Step 1

### Intermediate 349: 2-bromo-5-iodopyridin-4-amine

To a solution of 2-bromo-6-methylpyridin-4-amine (CAS: 79055-59-7, 4 g, 21.4 mmol) in MeCN (15 mL) was added NIS (5.77 g, 25.7 mmol). The reaction mixture was stirred overnight at 80° C. and then quenched with water (200 mL). The resulting solution was extracted three times with EtOAc and the combined organic layers were washed with brine, dried over anhydrous Na₂SO₄, filtered, and concentrated under reduced pressure. The crude material was purified by flash chromatography on silica gel using petroleum ether/EtOAc (9/91) as eluent to provide 6-bromo-3-iodo-2-methylpyridin-4-amine (Intermediate 349: 1.1 g, 16%) as a white solid. Mass spec: m/z: 313 [M+H]⁺.

### Step 2

### Intermediate 350: rac-tert-butyl 2-[2-(4-amino-6-bromo-2-methylpyridin-3-yl)ethynyl]pyrrolidine-1-carboxylate

A solution of 6-bromo-3-iodo-2-methylpyridin-4-amine (Intermediate 349, 1.1 g, 3.52 mmol), rac-tert-butyl 2-ethynylpyrrolidine-1-carboxylate (CAS: 316141-37-4, 824 mg, 4.22 mmol), Pd(PPh₃)₂Cl₂ (247 mg, 0.352 mmol), CuI (66.9 mg, 0.352 mmol) and Et₃N (1.07 g, 10.5 mmol) in DMF (15 mL) was stirred overnight at rt under nitrogen atmosphere. The mixture was concentrated under reduced pressure and the residue was chromatographed by reverse-phase flash chromatography (C18) using MeCN/H₂O (38/62) to provide rac-tert-butyl 2-[2-(4-amino-6-bromo-2-methylpyridin-3-yl)ethynyl]pyrrolidine-1-carboxylate (Intermediate 350, 250 mg, 16%) as a brown oil.

Mass spec: m/z: 380 [M+H]⁺.

### Step 3

### Intermediate 351: rac-tert-butyl 2-{6-bromo-4-methyl-1H-pyrrolo[3,2-c]pyridin-2-yl}pyrrolidine-1-carboxylate)

To a solution of rac-tert-butyl 2-[2-(4-amino-6-bromo-2-methylpyridin-3-yl)ethynyl]pyrrolidine-1-carboxylate (Intermediate 350: 250 mg, 0.657 mmol) in NMP (5 mL) was added a solution of tBuOK (1.97 mL, 1.97 mmol, 1M in THF). The reaction mixture was stirred for 4 h at 80° C., then quenched with water. The resulting solution was extracted three times with EtOAc and the combined organic layers washed with brine, dried over anhydrous Na₂SO₄, filtered, and concentrated under reduced pressure. The crude material was purified by flash chromatography on silica gel using petroleum ether/EtOAc (1/1) as eluent to provide rac-tert-butyl 2-{6-bromo-4-methyl-1H-pyrrolo[3,2-c]pyridin-2-yl}pyrrolidine-1-carboxylate) (Intermediate 351, 130 mg, 55%) as a colorless oil. Mass spec: m/z: 380 [M+H]⁺.

### Step 4

### Intermediate 352: rac-tert-butyl 2-[6-(1,3-dimethylindazole-6-amido)-4-methyl-1H-pyrrolo[3,2-c]pyridin-2-yl]pyrrolidine-1-carboxylate

A solution of rac-tert-butyl 2-{6-bromo-4-methyl-1H-pyrrolo[3,2-c]pyridin-2-yl}pyrrolidine-1-carboxylate (Intermediate 351, 130 mg, 0.342 mmol), 1,3-dimethylindazole-6-carboxamide (Intermediate 327, 77.6 mg, 0.410 mmol), GPhos (18.35 mg, 0.034 mmol), GPhos Pd G6 TES (32.3 mg, 0.034 mmol) and Cs₂CO₃ (334 mg, 1.03 mmol) in 1,4-dioxane (3 mL) was stirred for 3 h at 100° C. under nitrogen atmosphere. The reaction mixture was concentrated under reduced pressure and the crude material was purified by flash chromatography on silica gel using DCM/MeOH (97/3) as eluent to provide rac-tert-butyl 2-[6-(1,3-dimethylindazole-6-amido)-4-methyl-1H-pyrrolo[3,2-c]pyridin-2-yl]pyrrolidine-1-carboxylate (Intermediate 352, 130 mg, 80%) as a brown oil. Mass spec: m/z: 489 [M+H]⁺.

### Step 5

### Intermediate 353: rac-1,3-dimethyl-N-[4-methyl-2-(pyrrolidin-2-yl)-1H-pyrrolo[3,2-c]pyridin-6-yl]indazole-6-carboxamide trifluoroacetate

To a solution of rac-tert-butyl 2-[6-(1,3-dimethylindazole-6-amido)-4-methyl-1H-pyrrolo[3,2-c]pyridin-2-yl]pyrrolidine-1-carboxylate (Intermediate 352, 130 mg, 0.266 mmol) in DCM (4 mL) was added TFA (0.8 mL). The reaction mixture was stirred for 2 h at rt, then concentrated under reduced pressure to provide rac-1,3-dimethyl-N-[4-methyl-2-(pyrrolidin-2-yl)-1H-pyrrolo[3,2-c]pyridin-6-yl]indazole-6-carboxamide trifluoroacetate (Intermediate 353, 100 mg) as a colorless oil which was used in Step 6 without further purification. Mass spec: m/z: 389 [M+H]⁺.

### Step 6

### Intermediate 354: rac-1,3-dimethyl-N-[4-methyl-2-(1-methylpyrrolidin-2-yl)-1H-pyrrolo[3,2-c]pyridin-6-yl]indazole-6-carboxamide

To a solution of rac-1,3-dimethyl-N-[4-methyl-2-(1-methylpyrrolidin-2-yl)-1H-pyrrolo[3,2-c]pyridin-6-yl]indazole-6-carboxamide (Intermediate 353: 100 mg, 0.257 mmol) in MeOH (4 mL) were added paraformaldehyde (38.7 mg, 1.29 mmol) and Et₃N (78.2 mg, 0.771 mmol). The reaction mixture was stirred for 1 h at rt prior addition of NaBH₃CN (48.5 mg, 0.771 mmol). The reaction mixture was stirred for 1 h at 60° C., then concentrated under reduced pressure. The residue was chromatographed by reverse-phase flash chromatography (C18) using MeCN/H₂O (35/65) to afford rac-1,3-dimethyl-N-[4-methyl-2-(1-methylpyrrolidin-2-yl)-1H-pyrrolo[3,2-c]pyridin-6-yl]indazole-6-carboxamide (Intermediate 354: 60 mg, 68%) as a brown oil. Mass spec: m/z: 403 [M+H]⁺.

### Step 7

### Example 92: 1,3-dimethyl-N-{4-methyl-2-[(rel-2R)-1-methylpyrrolidin-2-yl]-1H-pyrrolo[3,2-c]pyridin-6-yl}indazole-6-carboxamide and Example 93: 1,3-dimethyl-N-{4-methyl-2-[(rel-2S)-1-methylpyrrolidin-2-yl]-1H-pyrrolo[3,2-c]pyridin-6-yl}indazole-6-carboxamide

Rac-1,3-dimethyl-N-[4-methyl-2-(1-methylpyrrolidin-2-yl)-1H-pyrrolo[3,2-c]pyridin-6-yl]indazole-6-carboxamide (Intermediate 354, 60 mg) was separated into the enantiomeric Example 92 and Example 93 using the following conditions: column: ChiralPak IF 2×25 cm, 5.0 um; Eluent: MTBE (0.5% 2M NH₃ in MeOH)/EtOH 95/5; Flow rate: 17 mL/min to provide:

### Example 92: 1,3-dimethyl-N-{4-methyl-2-[(rel-2R)-1-methylpyrrolidin-2-yl]-1H-pyrrolo[3,2-c]pyridin-6-yl}indazole-6-carboxamide

First eluting compound (retention time=5.4 min)

Mass spec: m/z: 403 [M+H]⁺; ¹H NMR (400 MHz, DMSO-d₆) δ ppm 11.32 (s, 1H), 10.41 (s, 1H), 8.42 (s, 1H), 8.10 (s, 1H), 7.74-7.80 (m, 2H), 6.41 (s, 1H), 4.06 (s, 3H), 3.39-3.41 (m, 4H), 3.14-3.19 (m, 1H), 2.60 (s, 3H), 2.25-2.27 (m, 1H), 2.17 (s, 4H), 1.80-1.91 (m, 3H).

### Example 93: 1,3-dimethyl-N-{4-methyl-2-[(rel-2S)-1-methylpyrrolidin-2-yl]-1H-pyrrolo[3,2-c]pyridin-6-yl}indazole-6-carboxamide

Second eluting compound (retention time=10.6 min)

Mass spec: m/z: 403 [M+H]⁺; ¹H NMR (400 MHz, DMSO-d₆) δ ppm 11.32 (s, 1H), 10.41 (s, 1H), 8.42 (s, 1H), 8.10 (s, 1H), 7.74-7.78 (m, 2H), 6.41 (s, 1H), 4.06 (s, 3H), 3.39-3.41 (m, 4H), 3.14-3.16 (m, 1H), 2.60 (s, 3H), 2.25-2.27 (m, 1H), 2.17 (s, 4H), 1.80-1.91 (m, 3H).

### Example 94 was Synthesised Following Scheme 79

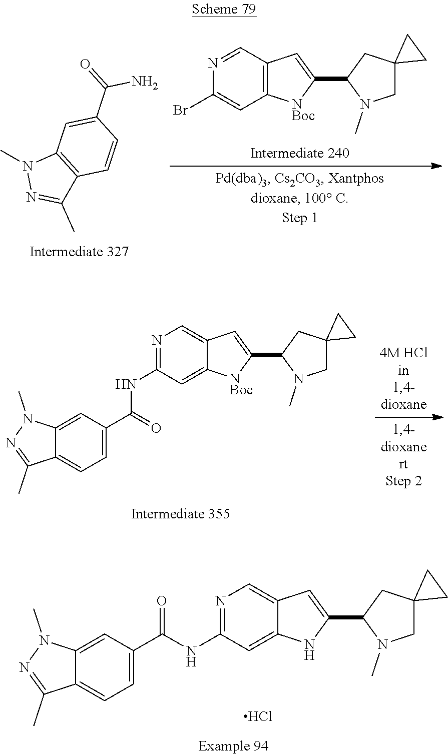

### Step 1

### Intermediate 356: tert-butyl (rel-R)-6-(1,3-dimethyl-1H-indazole-6-carboxamido)-2-(5-methyl-5-azaspiro [2.4]heptan-6-yl)-1H-pyrrolo[3,2-c]pyridine-1-carboxylate

To a solution of 1,3-dimethyl-1H-indazole-6-carboxamide (Intermediate 327, 0.19 g, 1.04 mmol) and tert-butyl (rel-R)-6-bromo-2-(5-methyl-5-azaspiro[2.4]heptan-6-yl)-1H-pyrrolo[3,2-c]pyridine-1-carboxylate (Intermediate 240, 0.40 g, 1.30 mmol) in 1,4-dioxane (8 mL) was added cesium carbonate (1.27 g, 3.91 mmol) at room temperature and the reaction mixture was purged with argon for 15 min. Pd₂(dba)₃ (0.18 g, 0.19 mmol) and Xantphos (0.23 g, 0.39 mmol) were then added and the reaction mixture was further purged with argon for 10 min and heated at 100° C. for 2 h. The reaction mixture was filtered through celite and washed with ethyl acetate (100 mL) and filtrate was concentrated in vacuo. The crude material was purified by combi-flash column chromatography by eluting with 2% MeOH in DCM to afford tert-butyl (rel-R)-6-(1,3-dimethyl-1H-indazole-6-carboxamido)-2-(5-methyl-5-azaspiro [2.4]heptan-6-yl)-1H-pyrrolo [3,2-c]pyridine-1-carboxylate (Intermediate 355, 0.30 g, 47%) as an off white solid. ¹H NMR (400 MHz, DMSO-d₆) δ ppm 0.35-0.65 (m, 4H), 1.09-1.19 (m, 1H), 1.58-1.67 (m, 1H), 1.71 (s, 9H), 2.35 (s, 3H), 2.45 (s, 3H), 2.61-2.65 (m, 1H), 2.77-2.82 (m, 1H), 4.05 (s, 3H), 4.16-4.21 (m, 1H), 6.86 (s, 1H), 7.71-7.75 (m, 1H), 7.78-7.83 (m, 1H), 8.42 (s, 1H), 8.63 (s, 1H), 8.98 (s, 1H), 10.74 (s, 1H). Mass spec: m/z: 415.3 [M-Boc]+.

### Step 2

### Example 94: (rel-R)-1,3-dimethyl-N-(2-(5-methyl-5-azaspiro [2.4]heptan-6-yl)-1H-pyrrolo[3,2-c]pyridin-6-yl)-1H-indazole-6-carboxamide hydrochloride

To a solution of tert-butyl (R)-6-(1,3-dimethyl-1H-indazole-6-carboxamido)-2-(5-methyl-5-azaspiro [2.4]heptan-6-yl)-1H-pyrrolo[3,2-c]pyridine-1-carboxylate (Intermediate 355, 0.30 g, 0.58 mmol) in 1,4-dioxane (6 mL) was added 4M HCl in 1,4-dioxane (5 mL) and the reaction mixture was stirred at room temperature for 3 h. The reaction mixture was concentrated in vacuo to afford (R)-1,3-dimethyl-N-(2-(5-methyl-5-azaspiro [2.4]heptan-6-yl)-1H-pyrrolo [3,2-c]pyridin-6-yl)-1H-indazole-6-carboxamide hydrochloride (Example 94, 0.05 g, 21%) as an off white solid. ¹H NMR (400 MHz, DMSO-d₆) δ ppm 0.51-0.75 (m, 4H), 2.01-2.14 (m, 2H), 2.18 (s, 3H), 2.54 (s, 3H), 2.56-2.58 (m, 1H), 2.89-2.93 (m, 1H), 3.61-3.65 (m, 1H), 4.07 (s, 3H), 6.46 (s, 1H), 7.74-7.78 (m, 1H), 7.79-7.83 (m, 1H), 8.27 (s, 1H), 8.44 (s, 1H), 8.55 (s, 1H), 10.53 (s, 1H), 11.49 (s, 1H). Mass spec: m/z: 413.2 [M−H]—.

### Example 95 was Synthesised Following Scheme 80

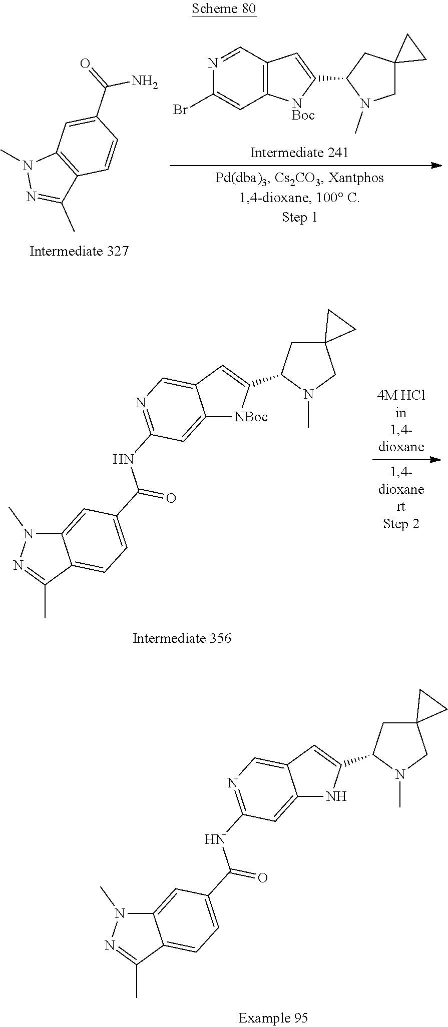 [SMILES: Cc1nn(C)c2cc(C(N)=O)ccc12 ; CN1CC2(CC2)C[C@H]1c1cc2cnc(Br)cc2n1C(=O)OC(C)(C)C ; Cc1nn(C)c2cc(C(=O)Nc3cc4c(cn3)cc([C@@H]3CC5(CC5)CN3C)n4C(=O)OC(C)(C)C)ccc12 ; Cc1nn(C)c2cc(C(=O)Nc3cc4[nH]c([C@@H]5CC6(CC6)CN5C)cc4cn3)ccc12]

### Step 1

### Intermediate 356: tert-butyl (rel-S)-6-(1,3-dimethyl-1H-indazole-6-carboxamido)-2-(5-methyl-5-azaspiro [2.4]heptan-6-yl)-1H-pyrrolo[3,2-c]pyridine-1-carboxylate

Intermediate 356 was synthesised in analogous manner to Intermediate 355, following Step 1 of Scheme 79, using 1,3-dimethyl-1H-indazole-6-carboxamide (Intermediate 327) as the amide and tert-butyl (rel-S)-6-bromo-2-(5-methyl-5-azaspiro[2.4]heptan-6-yl)-1H-pyrrolo[3,2-c]pyridine-1-carboxylate (Intermediate 241) as the aryl halide, to afford tert-butyl (rel-S)-6-(1,3-dimethyl-1H-indazole-6-carboxamido)-2-(5-methyl-5-azaspiro [2.4]heptan-6-yl)-1H-pyrrolo[3,2-c]pyridine-1-carboxylate (Intermediate 356). 1H NMR (400 MHz, DMSO-d6) δ ppm 0.40-0.65 (m, 4H), 1.09-1.19 (m, 1H), 1.58-1.67 (m, 1H), 1.71 (s, 9H), 2.35 (s, 3H), 2.45 (s, 3H), 2.61-2.65 (m, 1H), 2.77-2.82 (m, 1H), 4.05 (s, 3H), 4.15-4.21 (m, 1H), 6.86 (s, 1H), 7.71-7.75 (m, 1H), 7.78-7.83 (m, 1H), 8.42 (s, 1H), 8.64 (s, 1H), 8.98 (s, 1H), 10.74 (s, 1H). Mass spec: m/z: 515.2 [M+H]⁺.

### Step 2

### Example 95: (rel-S)-1,3-dimethyl-N-(2-(5-methyl-5-azaspiro[2.4]heptan-6-yl)-1H-pyrrolo[3,2-c]pyridin-6-yl)-1H-indazole-6-carboxamide

To a solution of tert-butyl (rel-S)-6-(1,3-dimethyl-1H-indazole-6-carboxamido)-2-(5-methyl-5-azaspiro[2.4]heptan-6-yl)-1H-pyrrolo[3,2-c]pyridine-1-carboxylate (Intermediate 356, 0.26 g, 0.63 mmol) in 1,4-dioxane (5.0 mL) was added 4M hydrochloric acid in 1,4-dioxane (5.0 mL) and the reaction mixture was stirred at room temperature for 5 h. The reaction mixture was concentrated in vacuo to obtain crude compound. The crude material was purified by preparative HPLC (Method A) to afford (rel-S)-1,3-dimethyl-N-(2-(5-methyl-5-azaspiro[2.4]heptan-6-yl)-1H-pyrrolo[3,2-c]pyridin-6-yl)-1H-indazole-6-carboxamide (Example 95, 0.055 g, 21%) as an off-white solid. 1H NMR (400 MHz, DMSO-d6) δ ppm 0.50-0.59 (m, 2H), 0.60-0.72 (m, 2H), 2.00-2.09 (m, 2H), 2.17 (s, 3H), 2.51 (s, 3H), 2.52-2.54 (m, 1H), 2.88-2.94 (m, 1H), 3.58-3.64 (m, 1H), 4.05 (s, 3H), 6.44 (s, 1H), 7.72-7.76 (m, 1H), 7.78-7.81 (m, 1H), 8.25 (s, 1H), 8.42 (s, 1H), 8.53 (s, 1H), 10.51 (s, 1H), 11.47 (br s, 1H). Mass spec: m/z: 415.2 [M+H]⁺.

### Example 96 was Synthesised Following Scheme 81

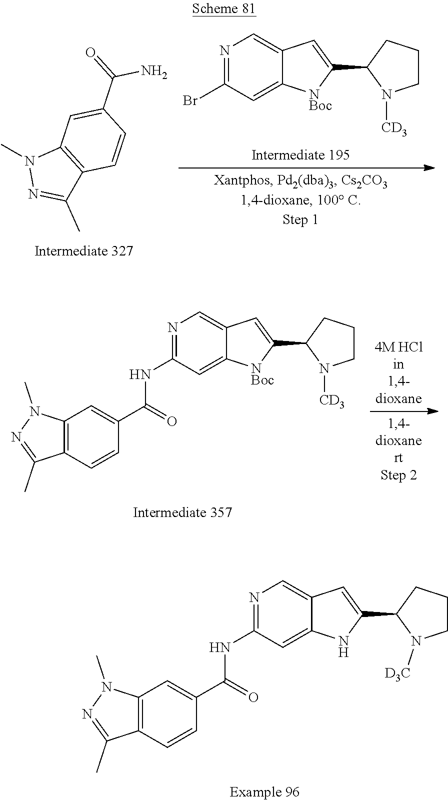 [SMILES: Cc1nn(C)c2cc(C(N)=O)ccc12 ; [2H]C([2H])([2H])N1CCC[C@@H]1c1cc2cnc(Br)cc2n1C(=O)OC(C)(C)C ; [2H]C([2H])([2H])N1CCC[C@@H]1c1cc2cnc(NC(=O)c3ccc4c(C)nn(C)c4c3)cc2n1C(=O)OC(C)(C)C ; [2H]C([2H])([2H])N1CCC[C@@H]1c1cc2cnc(NC(=O)c3ccc4c(C)nn(C)c4c3)cc2[nH]1]

### Step 1

### Intermediate 357: tert-butyl (R)-6-(1,3-dimethyl-1H-indazole-6-carboxamido)-2-(1-(methyl-d3) pyrrolidin-2-yl)-1H-pyrrolo [3,2-c]pyridine-1-carboxylate

To a solution of 1,3-dimethyl-1H-indazole-6-carboxamide (Intermediate 327, 0.098 g, 0.52 mmol) in 1,4-dioxane (5 mL) was added tert-butyl (R)-6-bromo-2-(1-(methyl-d3)pyrrolidin-2-yl)-1H-pyrrolo[3,2-c]pyridine-1-carboxylate (Intermediate 195, 0.25 g, 0.65 mmol) followed by cesium carbonate (0.64 g, 1.96 mmol) at room temperature. The reaction mixture was purged with argon for 15 min then Pd₂(dba)₃ (0.061 g, 0.065 mmol) and Xantphos (0.12 g, 0.19 mmol) were added. The reaction mixture was further purged with argon for 10 min and then stirred at 100° C. for 2 h. The reaction mixture was filtered through celite and washed with ethyl acetate (50 mL) and concentrated in vacuo. The crude material was purified by combi-flash chromatography by eluting with 90% ethyl acetate in heptane to afford tert-butyl (R)-6-(1,3-dimethyl-1H-indazole-6-carboxamido)-2-(1-(methyl-d3)pyrrolidin-2-yl)-1H-pyrrolo[3,2-c]pyridine-1-carboxylate (Intermediate 357, 0.20 g, 62%) as a yellow solid. ¹H NMR (400 MHz, DMSO-d₆) δ ppm 1.57-1.65 (m, 1H), 1.71 (s, 9H), 1.74-1.81 (m, 2H), 2.31-2.43 (m, 2H), 2.51 (s, 3H), 3.09-3.17 (m, 1H), 3.90-3.96 (m, 1H), 4.05 (s, 3H), 6.76 (s, 1H), 7.73 (d, J=8.40 Hz, 1H), 7.80 (d, J=8.40 Hz, 1H), 8.41 (s, 1H), 8.61 (s, 1H), 8.98 (s, 1H), 10.74 (s, 1H). Mass spec: m/z: 492.0 [M+H]⁺.

### Step 2

### Example 96: (R)-1,3-dimethyl-N-(2-(1-(methyl-d₃)pyrrolidin-2-yl)-1H-pyrrolo[3,2-c]pyridin-6-yl)-1H-indazole-6-carboxamide

To a solution of tert-butyl (R)-6-(1,3-dimethyl-1H-indazole-6-carboxamido)-2-(1-(methyl-d₃) pyrrolidin-2-yl)-1H-pyrrolo[3,2-c]pyridine-1-carboxylate (Intermediate 357, 0.20 g, 0.40 mmol) in 1,4-dioxane (4 mL) was added 4M hydrochloric acid in 1,4-dioxane (4 mL) at 0° C. and the reaction mixture was stirred at room temperature for 3 h. The reaction mixture was concentrated in vacuo to obtain the crude compound. The crude material was purified by preparative HPLC (Method A) to afford (R)-1,3-dimethyl-N-(2-(1-(methyl-d₃) pyrrolidin-2-yl)-1H-pyrrolo [3,2-c]pyridin-6-yl)-1H-indazole-6-carboxamide (Example 96, 0.045 g, 28%) as an off-white solid. ¹H NMR (400 MHz, DMSO-d₆) δ ppm 1.77-1.96 (m, 3H), 2.11-2.20 (m, 1H), 2.22-2.31 (m, 1H), 2.52 (s, 3H), 3.11-3.17 (m, 1H), 3.32-3.35 (m, 1H), 4.05 (s, 3H), 6.40 (s, 1H), 7.74 (d, J=8.40 Hz, 1H), 7.81 (d, J=8.40 Hz, 1H), 8.25 (s, 1H), 8.42 (s, 1H), 8.52 (s, 1H), 10.50 (s, 1H), 11.38 (s, 1H). Mass spec: m/z: 392.0 [M+H]⁺.

### Example 97 was Synthesised Following Scheme 82

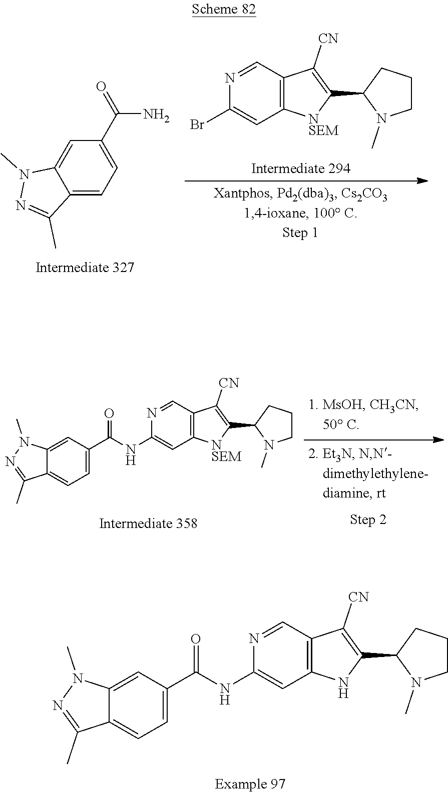 [SMILES: Cc1nn(C)c2cc(C(N)=O)ccc12 ; CN1CCC[C@@H]1c1c(C#N)c2cnc(Br)cc2n1COCC[Si](C)(C)C ; Cc1nn(C)c2cc(C(=O)Nc3cc4c(cn3)c(C#N)c([C@H]3CCCN3C)n4COCC[Si](C)(C)C)ccc12 ; Cc1nn(C)c2cc(C(=O)Nc3cc4[nH]c([C@H]5CCCN5C)c(C#N)c4cn3)ccc12]

### Step 1

### Intermediate 358: (R)—N-(3-cyano-2-(1-methylpyrrolidin-2-yl)-1-((2-(trimethylsilyl) ethoxy) methyl)-1H-pyrrolo [3,2-c]pyridin-6-yl)-1,3-dimethyl-1H-indazole-6-carboxamide

To a solution of (R)-6-bromo-2-(1-methylpyrrolidin-2-yl)-1-((2-(trimethylsilyl)ethoxy)methyl)-1H-pyrrolo[3,2-c]pyridine-3-carbonitrile (Intermediate 294, 0.16 g, 0.37 mmol) in 1,4-dioxane (2.0 mL) was added 1,3-dimethyl-1H-indazole-6-carboxamide (Intermediate 327, 0.05 g, 0.29 mmol) followed by cesium carbonate (0.36 g, 1.1 mmol) at room temperature. The reaction mixture was purged with argon for 15 min then Pd₂(dba)₃ (0.05 g, 0.05 mmol) and Xantphos (0.06 g, 0.11 mmol) were added. The reaction mixture was further purged with argon for 10 min and then stirred at 100° C. for 2 h. The reaction mixture was filtered through celite, washed with ethyl acetate (100 mL) and filtrate was concentrated in vacuo. The crude material was purified by combi-flash chromatography by eluting with 90% ethyl acetate in heptane to afford (R)—N-(3-cyano-2-(1-methylpyrrolidin-2-yl)-1-((2-(trimethylsilyl) ethoxy) methyl)-1H-pyrrolo[3,2-c]pyridin-6-yl)-1,3-dimethyl-1H-indazole-6-carboxamide (Intermediate 358, 0.03 g, 20%) as a yellow solid. ¹H NMR (400 MHz, DMSO-d₆) δ ppm −0.06 (s, 9H), 0.87-0.93 (m, 2H), 1.82-1.98 (m, 4H), 2.28 (s, 3H), 2.32-2.41 (m, 2H), 3.16-3.22 (m, 1H), 3.57 (s, 3H), 3.59-3.65 (m, 2H), 4.06 (s, 3H), 5.69-5.83 (m, 2H), 7.74 (d, J=8.40, 1H), 7.81 (d, J=8.40 Hz, 1H), 8.46 (s, 1H), 8.61 (s, 1H), 8.72 (s, 1H), 10.93 (br s, 1H). Mass spec: m/z: 544.3 [M+H]⁺.

### Step 2

### Example 97: (R)—N-(3-cyano-2-(1-methylpyrrolidin-2-yl)-1H-pyrrolo [3,2-c]pyridin-6-yl)-1,3-dimethyl-1H-indazole-6-carboxamide

To a solution of (R)—N-(3-cyano-2-(1-methylpyrrolidin-2-yl)-1-((2-(trimethylsilyl) ethoxy) methyl)-1H-pyrrolo [3,2-c]pyridin-6-yl)-1,3-dimethyl-1H-indazole-6-carboxamide (Intermediate 358, 0.03 g, 0.06 mmol) in acetonitrile (2.0 mL) was added methanesulfonic acid (0.05 mL, 0.8 mmol) at room temperature and the reaction mixture was stirred at 50° C. for 2h. After completion of the reaction, the mixture was cooled to room temperature then N,N′-dimethylethylenediamine (0.05 mL, 0.4 mmol) followed by triethylamine (0.2 mL, 1 mmol) were added and stirred at room temperature for 3 h. The reaction mixture was concentrated in vacuo, the obtained residue was poured into ice cold water (40 mL) and extracted with ethyl acetate (3×50 mL). The organic layer was separated, dried over anhydrous Na₂SO₄, and concentrated in vacuo. The crude material was purified by preparative HPLC (Method A) to afford (R)—N-(3-cyano-2-(1-methylpyrrolidin-2-yl)-1H-pyrrolo [3,2-c]pyridin-6-yl)-1,3-dimethyl-1H-indazole-6-carboxamide (Example 97, 0.006 g, 30%) as an off-white solid. ¹H NMR (400 MHz, DMSO-d₆) δ ppm 1.84-2.01 (m, 3H), 2.24 (s, 3H), 2.28-2.34 (m, 1H), 2.37-2.44 (m, 1H), 2.52 (s, 3H), 3.17-3.24 (m, 1H), 3.56-3.65 (m, 1H), 4.06 (s, 3H), 7.75 (d, J=8.40 Hz, 1H), 7.81 (d, J=8.40 Hz, 1H), 8.41 (s, 1H), 8.44 (s, 1H), 8.68 (s, 1H), 10.80 (br s, 1H). Mass spec: m/z: 414.2 [M+H]⁺.

### Examples 98 and 99 were Prepared Following Scheme 83

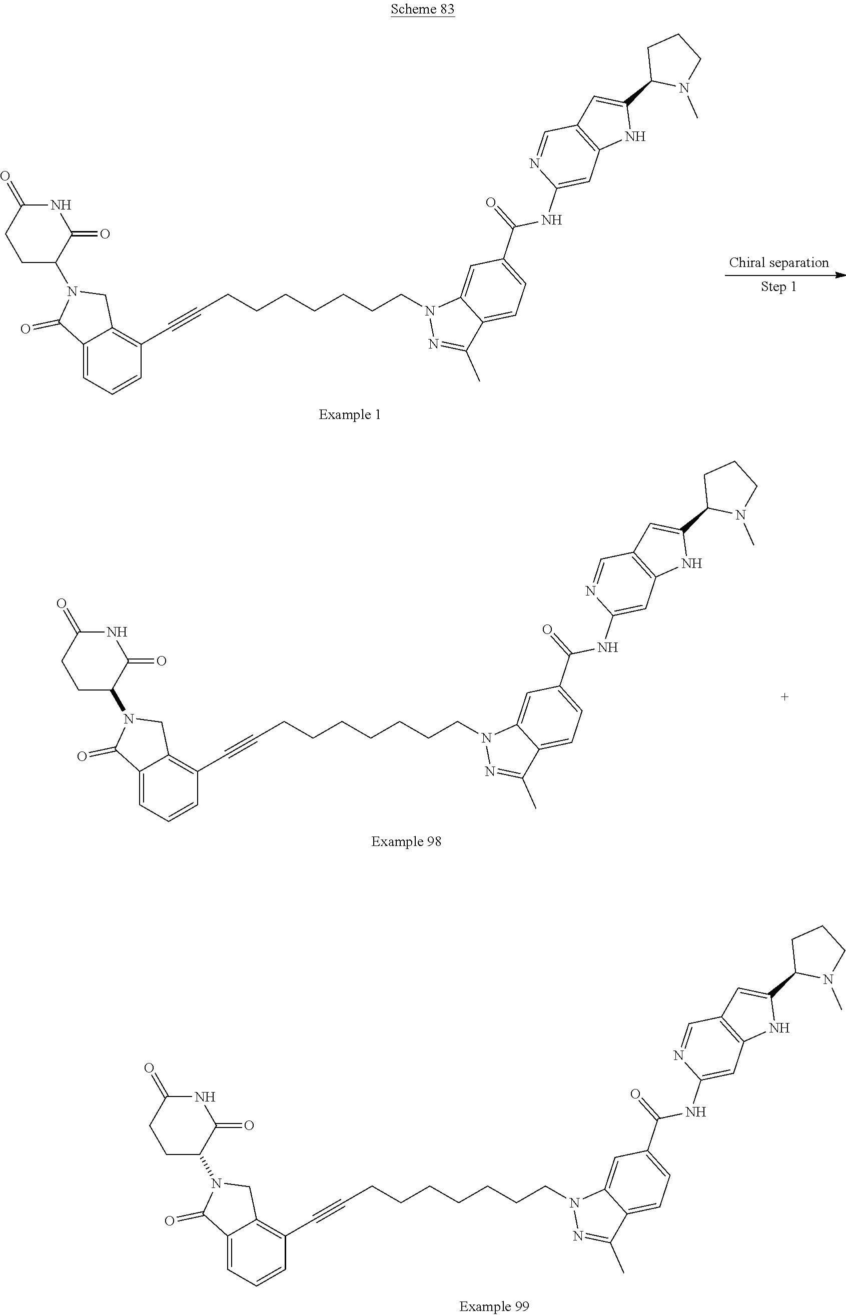 [SMILES: Cc1nn(CCCCCCCC#Cc2cccc3c2CN(C2CCC(=O)NC2=O)C3=O)c2cc(C(=O)Nc3cc4[nH]c([C@H]5CCCN5C)cc4cn3)ccc12 ; Cc1nn(CCCCCCCC#Cc2cccc3c2CN([C@H]2CCC(=O)NC2=O)C3=O)c2cc(C(=O)Nc3cc4[nH]c([C@H]5CCCN5C)cc4cn3)ccc12 ; Cc1nn(CCCCCCCC#Cc2cccc3c2CN([C@@H]2CCC(=O)NC2=O)C3=O)c2cc(C(=O)Nc3cc4[nH]c([C@H]5CCCN5C)cc4cn3)ccc12]

### Step 1

### Example 98: 1-(9-(2-((rel-S)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

### and Example 99: 1-(9-(2-((rel-R)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

1-(9-(2-(2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 1, 0.30 g, 0.40 mmol) was separated by chiral preparative HPLC (Column: RR WHELK (30λ250*mm), 5 M; Mobile Phase A: DCM, Mobile Phase B: iPrOH; Flow rate: 40 ml/min; isocratic 60% B) to afford 1-(9-(2-((rel-S)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 98, 1st eluting peak, 0.04 g, 16%) as an off-white solid and 1-(9-(2-((rel-R)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide (Example 99, 2nd eluting peak, 0.03 g, 13%) as an off-white solid.

### Example 98: 1-(9-(2-((rel-S)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

1st eluting peak from Chiral Preparative HPLC. Retention time: 4.32 mins.

¹H NMR (401 MHz, DMSO-d₆) δ ppm 1.23-1.38 (m, 7H), 1.50-1.57 (m, 2H), 1.79-2.00 (m, 7H), 2.17 (s, 3H), 2.23-2.30 (m, 1H), 2.41-2.45 (m, 1H), 2.46 (s, 3H), 2.54-2.58 (m, 2H), 2.85-2.96 (m, 1H), 3.12-3.19 (m, 1H), 3.29-3.33 (m, 1H), 4.27-4.32 (m, 1H), 4.40-4.45 (m, 3H), 5.11-5.16 (m, 1H), 6.41 (s, 1H), 7.46-7.53 (m, 1H), 7.60 (d, J=7.70 Hz, 1H), 7.67-7.76 (m, 2H), 7.76-7.81 (m, 1H), 8.25 (s, 1H), 8.44 (s, 1H), 8.52 (s, 1H), 10.56 (br s, 1H), 11.03 (br s, 1H), 11.41 (br s, 1H). Mass spec m/z 739.4 [M+H]⁺.

### Example 99: 1-(9-(2-((rel-R)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide

2nd eluting peak from Chiral Preparative HPLC. Retention time: 5.16 mins.

¹H NMR (401 MHz, DMSO-d₆) δ ppm 1.23-1.39 (m, 7H), 1.50-1.59 (m, 2H), 1.79-2.02 (m, 7H), 2.17 (s, 3H), 2.24-2.30 (m, 1H), 2.41-2.45 (m, 1H), 2.46 (s, 3H), 2.54-2.58 (m, 2H), 2.85-2.96 (m, 1H), 3.12-3.19 (m, 1H), 3.29-3.33 (m, 1H), 4.27-4.32 (m, 1H), 4.40-4.45 (m, 3H), 5.11-5.16 (m, 1H), 6.41 (s, 1H), 7.47-7.52 (m, 1H), 7.57-7.63 (m, 1H), 7.68-7.79 (m, 3H), 8.25 (s, 1H), 8.44 (s, 1H), 8.52 (s, 1H), 10.56 (br s, 1H), 11.03 (br s, 1H), 11.41 (br s, 1H). Mass spec m/z 739.4 [M+H]⁺.

### Examples 100 and 101 were Prepared Following Scheme 84

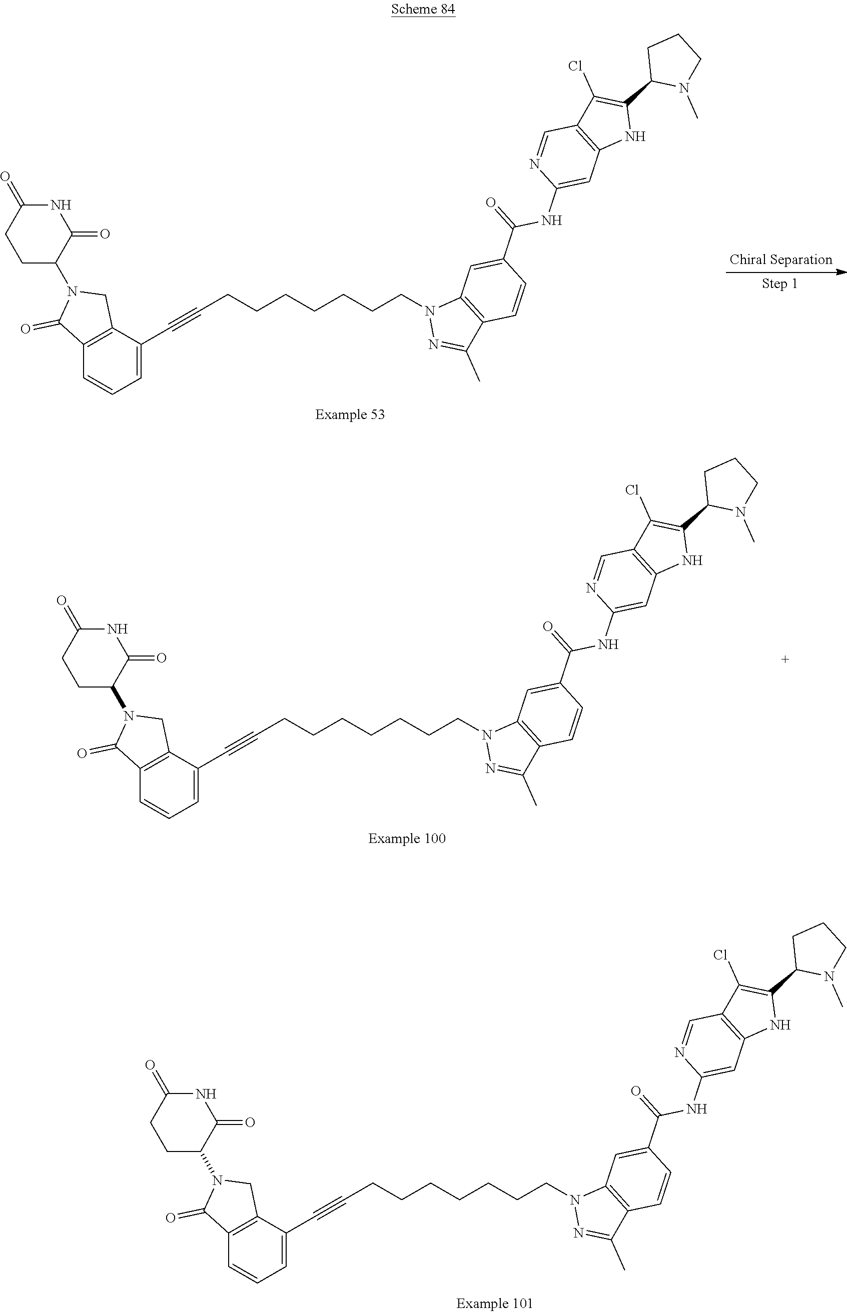 [SMILES: Cc1nn(CCCCCCCC#Cc2cccc3c2CN(C2CCC(=O)NC2=O)C3=O)c2cc(C(=O)Nc3cc4[nH]c([C@H]5CCCN5C)c(Cl)c4cn3)ccc12 ; Cc1nn(CCCCCCCC#Cc2cccc3c2CN([C@H]2CCC(=O)NC2=O)C3=O)c2cc(C(=O)Nc3cc4[nH]c([C@H]5CCCN5C)c(Cl)c4cn3)ccc12 ; Cc1nn(CCCCCCCC#Cc2cccc3c2CN([C@@H]2CCC(=O)NC2=O)C3=O)c2cc(C(=O)Nc3cc4[nH]c([C@H]5CCCN5C)c(Cl)c4cn3)ccc12]

### Step 1

### Example 100: N-(3-chloro-2-((R)-1-methylpyrrolidin-2-yl)-1H-pyrrolo[3,2-c]pyridin-6-yl)-1-(9-(2-((rel-S)-2,6-dioxopiperidin-3-yl)-1-oxoisoindolin-4-yl)non-8-yn-1-yl)-3-methyl-1H-indazole-6-carboxamide

### and Example 101: N-(3-chloro-2-((R)-1-methylpyrrolidin-2-yl)-1H-pyrrolo[3,2-c]pyridin-6-yl)-1-(9-(2-((rel-R)-2,6-dioxopiperidin-3-yl)-1-oxoisoindolin-4-yl)non-8-yn-1-yl)-3-methyl-1H-indazole-6-carboxamide

N-(3-chloro-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide (Example 53, 0.25 g, 0.32 mmol) was separated by chiral preparative HPLC (CHIRALPAK IK (50×250*mm), 5 m; Mobile Phase A: DCM, Mobile Phase B: DCM: iProH (1:1); Flow rate: 90 ml/min; isocratic 50% B) to afford N-(3-chloro-2-((R)-1-methylpyrrolidin-2-yl)-1H-pyrrolo[3,2-c]pyridin-6-yl)-1-(9-(2-((rel-S)-2,6-dioxopiperidin-3-yl)-1-oxoisoindolin-4-yl)non-8-yn-1-yl)-3-methyl-1H-indazole-6-carboxamide (Example 100, 1st eluting peak, 0.081 g, 32%) as an off-white solid and N-(3-chloro-2-((R)-1-methylpyrrolidin-2-yl)-1H-pyrrolo[3,2-c]pyridin-6-yl)-1-(9-(2-((rel-R)-2,6-dioxopiperidin-3-yl)-1-oxoisoindolin-4-yl)non-8-yn-1-yl)-3-methyl-1H-indazole-6-carboxamide (Example 101, 2nd eluting peak, 0.073 g, 29%) as an off-white solid.

### Example 100: N-(3-chloro-2-((R)-1-methylpyrrolidin-2-yl)-1H-pyrrolo[3,2-c]pyridin-6-yl)-1-(9-(2-((rel-S)-2,6-dioxopiperidin-3-yl)-1-oxoisoindolin-4-yl)non-8-yn-1-yl)-3-methyl-1H-indazole-6-carboxamide

1^{st} eluting peak from Chiral Preparative HPLC. Retention time: 3.64 min.

¹H NMR (400 MHz, DMSO-d₆) δ ppm 1.25-1.45 (m, 6H), 1.50-1.60 (m, 2H), 1.81-1.91 (m, 4H), 1.91-2.01 (m, 2H), 2.15-2.21 (m, 4H), 2.24-2.34 (m, 1H), 2.39-2.51 (m, 5H), 2.51-2.61 (m, 2H), 2.82-2.95 (m, 1H), 3.17 (t, J=7.6 Hz, 1H), 3.52 (t, J=8 Hz, 1H), 4.25-4.32 (m, 1H), 4.35-4.44 (m, 3H), 5.13 (dd, J=13.2, 5.2 Hz, 1H), 7.45-7.51 (m, 1H), 7.55-7.61 (m, 1H), 7.65-7.8 (m, 3H), 8.30 (s, 1H), 8.44 (s, 1H), 8.51 (s, 1H), 10.68 (br s, 1H), 10.99 (br s, 1H), 11.71 (br s, 1H). Mass spec m/z 773.3 [M+H]⁺.

### Example 101: N-(3-chloro-2-((R)-1-methylpyrrolidin-2-yl)-1H-pyrrolo[3,2-c]pyridin-6-yl)-1-(9-(2-((rel-R)-2,6-dioxopiperidin-3-yl)-1-oxoisoindolin-4-yl)non-8-yn-1-yl)-3-methyl-1H-indazole-6-carboxamide

2nd eluting peak from Chiral Preparative HPLC. Retention time: 4.69 min.

¹H NMR (400 MHz, DMSO-d₆) δ ppm 1.25-1.45 (m, 6H), 1.51-1.60 (m, 2H), 1.81-2.05 (m, 6H), 2.13-2.21 (m, 4H), 2.24-2.35 (m, 1H), 2.38-2.51 (m, 5H), 2.51-2.61 (m, 2H), 2.82-2.95 (m, 1H), 3.17 (t, J=7.6 Hz, 1H), 3.52 (t, J=8 Hz, 1H), 4.25-4.32 (m, 1H), 4.35-4.45 (m, 3H), 5.13 (dd, J=13.2, 5.2 Hz, 1H), 7.45-7.51 (m, 1H), 7.56-7.60 (m, 1H), 7.66-7.81 (m, 3H), 8.31 (s, 1H), 8.44 (s, 1H), 8.51 (s, 1H), 10.68 (br s, 1H), 10.99 (br s, 1H), 11.71 (br s, 1H). Mass spec m/z 773.3 [M+H]⁺.

### Examples 102 and 103 were Prepared Following Scheme 85

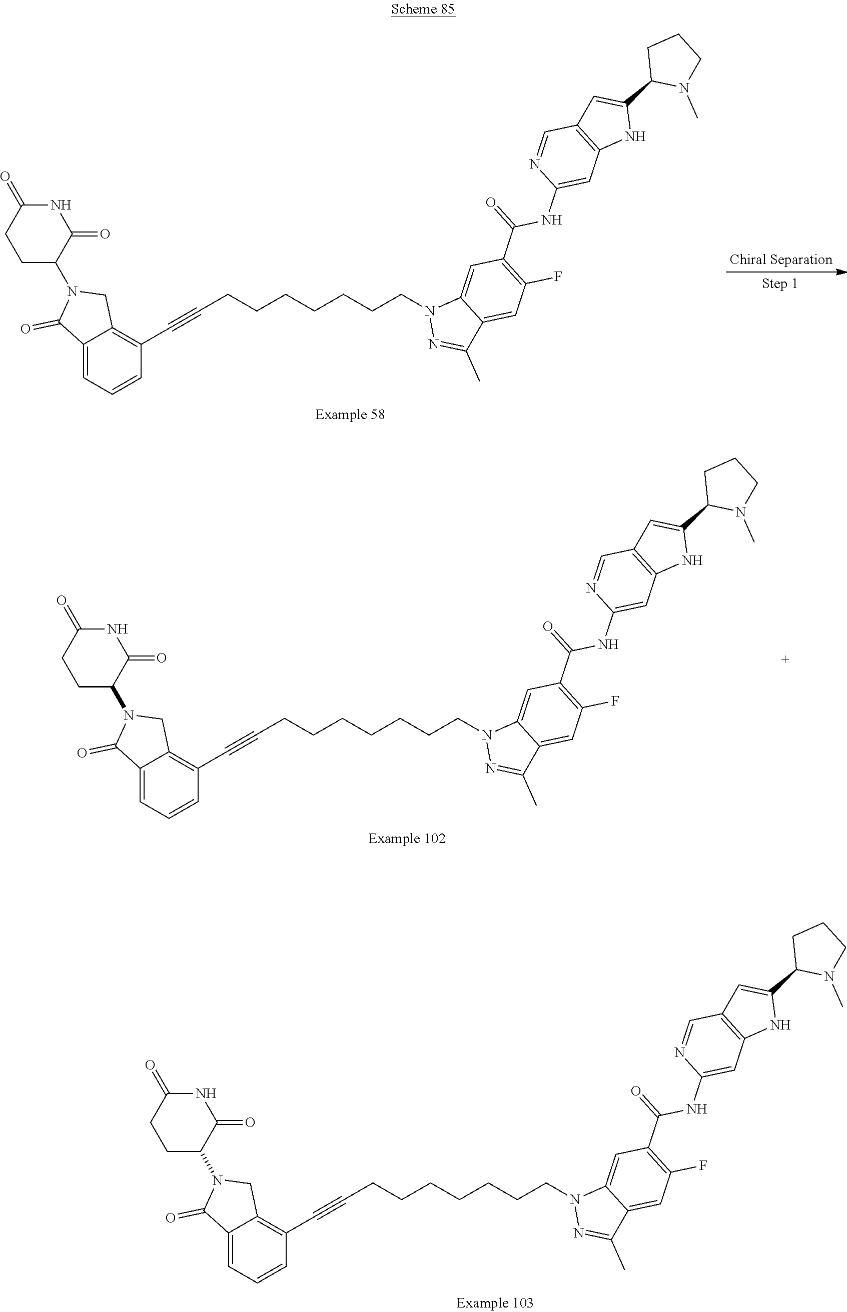 [SMILES: Cc1nn(CCCCCCCC#Cc2cccc3c2CN(C2CCC(=O)NC2=O)C3=O)c2cc(C(=O)Nc3cc4[nH]c([C@H]5CCCN5C)cc4cn3)c(F)cc12 ; Cc1nn(CCCCCCCC#Cc2cccc3c2CN([C@H]2CCC(=O)NC2=O)C3=O)c2cc(C(=O)Nc3cc4[nH]c([C@H]5CCCN5C)cc4cn3)c(F)cc12 ; Cc1nn(CCCCCCCC#Cc2cccc3c2CN([C@@H]2CCC(=O)NC2=O)C3=O)c2cc(C(=O)Nc3cc4[nH]c([C@H]5CCCN5C)cc4cn3)c(F)cc12]

### Step 1

### Example 102: 1-(9-(2-((rel-S)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide

### and Example 103: 1-(9-(2-((rel-R)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide

1-(9-(2-(2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide (Example 58, 4.0 g, 5.28 mmol) was separated by chiral preparative HPLC (Column: CHIRALPAK IK (50×250*mm), 5 m; Mobile Phase A: DCM, Mobile Phase B: DCM: iPrOH (1:1); Flow rate: 90 ml/min; isocratic 50% B) to afford 1-(9-(2-((rel-S)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide (Example 102, 1st eluting peak, 0.96 g, 24%) as an off-white solid and 1-(9-(2-((rel-R)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide (Example 103, 2nd eluting peak, 1.00 g, 25%) as an off-white solid.

### Example 102: 1-(9-(2-((rel-S)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide

1^{st} eluting peak from chiral preparative HPLC. Retention time: 4.02 mins.

¹H NMR (400 MHz, DMSO-d₆) δ ppm 1.27-1.39 (m, 6H), 1.51-.56 (m, 2H), 1.78-2.03 (m, 6H), 2.11-2.16 (m, 1H), 2.17 (s, 3H), 2.22-2.31 (m, 1H), 2.42-2.47 (m, 2H), 2.48 (s, 3H), 2.54-2.62 (m, 1H), 2.84-2.96 (m, 1H), 3.12-3.16 (m, 1H), 3.29-3.31 (m, 1H), 3.34-3.36 (m, 1H), 4.27-4.45 (m, 4H), 5.11-5.15 (m, 1H), 6.39 (s, 1H), 7.45-7.51 (m, 1H), 7.59-7.65 (m, 2H), 7.69 (dd, J=7.50, 0.75 Hz, 1H), 8.00 (s, 1H), 8.24 (s, 1H), 8.48 (s, 1H), 10.50 (br s, 1H), 11.00 (br s, 1H), 11.40 (br s, 1H). Mass spec m/z 757.5 [M+H]⁺.

### Example 103: 1-(9-(2-((rel-R)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide

2nd eluting peak from chiral preparative HPLC. Retention time: 5.42 mins.

¹H NMR (400 MHz, DMSO-d₆) δ ppm 1.27-1.39 (m, 6H), 1.50-1.56 (m, 2H), 1.78-2.00 (m, 6H), 2.11-2.16 (m, 1H), 2.17 (s, 3H), 2.22-2.31 (m, 1H), 2.37-2.44 (m, 2H), 2.45 (s, 3H), 2.54-2.62 (m, 1H), 2.84-2.96 (m, 1H), 3.12-3.16 (m, 1H), 3.29-3.31 (m, 1H), 3.34-3.36 (m, 1H), 4.27-4.45 (m, 4H), 5.10-5.15 (m, 1H), 6.39 (s, 1H), 7.47-7.51 (m, 1H), 7.59-7.63 (m, 2H), 7.68-7.70 (m, 1H), 8.00 (s, 1H), 8.24 (s, 1H), 8.48 (s, 1H), 10.50 (br s, 1H), 11.00 (br s, 1H), 11.40 (br s, 1H). Mass spec m/z 757.4 [M+H]⁺.

### Example 104 was Synthesised Following Scheme 86

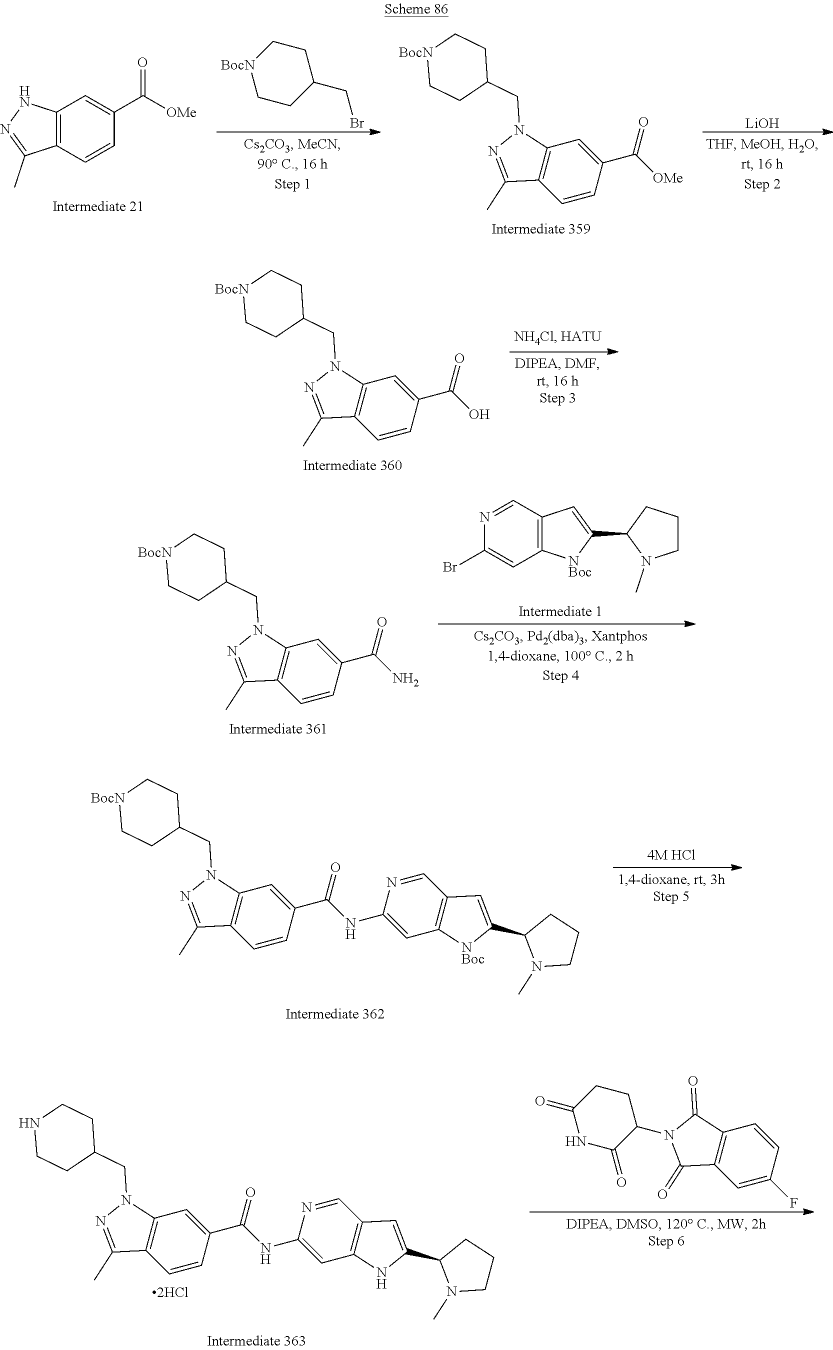

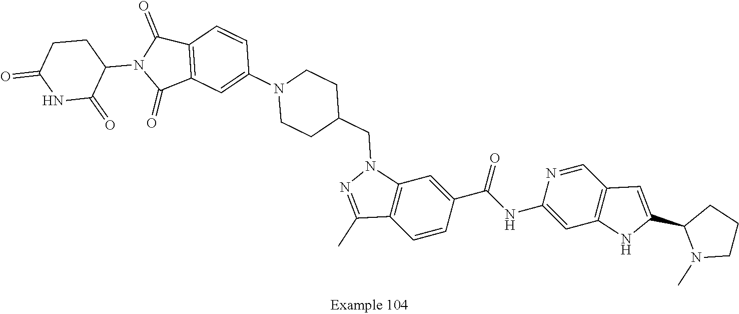 [SMILES: Cc1nn(CC2CCN(c3ccc4c(c3)C(=O)N(C3CCC(=O)NC3=O)C4=O)CC2)c2cc(C(=O)Nc3cc4[nH]c([C@H]5CCCN5C)cc4cn3)ccc12]

### Step 1

### Intermediate 359: methyl 1-((1-(tert-butoxycarbonyl)piperidin-4-yl)methyl)-3-methyl-1H-indazole-6-carboxylate

To a solution of methyl 3-methyl-1H-indazole-6-carboxylate (Intermediate 21, 4.0 g, 21.03 mmol) in acetonitrile (50 mL) was added tert-butyl 4-(bromomethyl)piperidine-1-carboxylate (CAS No: 158407-04-6, 6.43 g, 23.13 mmol) and cesium carbonate (20.6 g, 63.09 mmol) and the reaction mixture was heated at 90° C. for 16 h. After completion, the reaction mixture was diluted with water (100 mL) and extracted with ethyl acetate (3×150 mL). The combined organic layers were dried over anhydrous Na₂SO₄, filtered and concentrated in vacuo to obtain crude compound as a mixture of regioisomers. The regioisomers were separated by combi-flash chromatography, by eluting with 35% ethyl acetate in heptane, to afford desired isomer methyl 1-((1-(tert-butoxycarbonyl)piperidin-4-yl)methyl)-3-methyl-1H-indazole-6-carboxylate (Intermediate 359, 6.4 g, 79%) as a yellow sticky liquid. ¹H NMR (400 MHz, DMSO-d) 6 ppm 1.10-1.19 (m, 2H), 1.37 (s, 9H), 1.40-1.45 (m, 2H), 2.03-2.11 (m, 1H), 2.51 (s, 3H), 2.57-2.68 (m, 2H), 3.86-3.88 (m, 1H), 3.90 (s, 3H), 3.92-3.96 (m, 1H), 4.32 (d, J=7.0 Hz, 2H), 7.65 (d, J=8.29 Hz, 1H), 7.81 (d, J=8.29 Hz, 1H), 8.28 (s, 1H). Mass spec m/z 388.1 [M+H]⁺.

### Step 2

### Intermediate 360: 1-((1-(tert-butoxycarbonyl)piperidin-4-yl)methyl)-3-methyl-1H-indazole-6-carboxylic acid

To a solution of methyl 1-((1-(tert-butoxycarbonyl)piperidin-4-yl)methyl)-3-methyl-1H-indazole-6-carboxylate (Intermediate 359, 6.4 g, 17.0 mmol) in tetrahydrofuran (40 mL), methanol (40 mL) and water (20 mL) was added lithium hydroxide (1.6 g, 66.0 mmol) and the reaction mixture was stirred at room temperature for 16 h. After completion, the reaction mixture was concentrated in vacuo. The obtained residue was acidified to pH~5 with 1N aqueous HCl solution. The resultant precipitate was collected by filtration, washed with water (200 mL), heptane (200 mL) and dried in vacuo to afford 1-((1-(tert-butoxycarbonyl)piperidin-4-yl)methyl)-3-methyl-1H-indazole-6-carboxylic acid (Intermediate 360, 5.80 g, 94%) as an off-white solid, which was used for the next step without further purification. ¹H NMR (400 MHz, DMSO-d₆) δ ppm 1.09-1.17 (m, 2H), 1.36 (s, 9H), 1.39-1.45 (m, 2H), 2.02-2.11 (m, 1H), 2.51 (s, 3H), 2.58-2.68 (m, 2H), 3.88-3.92 (m, 2H), 4.31 (d, J=7.04 Hz, 2H), 7.64 (d, J=8.22 Hz, 1H), 7.77 (d, J=8.61 Hz, 1H), 8.24 (s, 1H), 12.98 (br s, 1H). Mass spec m/z 374.1 [M+H]⁺.

### Step 3

### Intermediate 361: tert-butyl 4-((6-carbamoyl-3-methyl-1H-indazol-1-yl)methyl)piperidine-1-carboxylate

To a cooled (0° C.) solution of 1-((1-(tert-butoxycarbonyl)piperidin-4-yl)methyl)-3-methyl-1H-indazole-6-carboxylic acid (Intermediate 360, 5.8 g, 16.0 mmol) in N,N-dimethylformamide (60 mL) was added HATU (9.1 g, 23.0 mmol) and N,N-diisopropylethylamine (8.1 mL, 47.0 mmol). Ammonium chloride (4.2 g, 78.0 mmol) was then added and the reaction stirred at room temperature for 16 h. After completion, the reaction mixture was diluted with ice cold water (250 mL), the resultant precipitate was collected by filtration and dried in vacuo to afford tert-butyl 4-((6-carbamoyl-3-methyl-1H-indazol-1-yl)methyl)piperidine-1-carboxylate (Intermediate 361, 5.0 g, 86%) as an off-white solid, which was used in the step without further purification. Mass spec m/z 371.1 [M+H]⁺.

### Step 4

### Intermediate 362: tert-butyl (R)-6-(1-((1-(tert-butoxycarbonyl)piperidin-4-yl)methyl)-3-methyl-1H-indazole-6-carboxamido)-2-(1-methylpyrrolidin-2-yl)-1H-pyrrolo[3,2-c]pyridine-1-carboxylate

To a solution of tert-butyl 4-((6-carbamoyl-3-methyl-1H-indazol-1-yl) methyl)piperidine-1-carboxylate (Intermediate 361, 0.70 g, 1.87 mmol) in 1,4-dioxane (15 mL) was added tert-butyl (R)-6-bromo-2-(1-methylpyrrolidin-2-yl)-1H-pyrrolo[3,2-c]pyridine-1-carboxylate (Intermediate 1, 0.85 g, 2.25 mmol) followed by cesium carbonate (1.84 g, 5.63 mmol). The reaction mixture was purged with argon for 15 min, followed by the addition of Pd₂(dba)₃ (0.26 g, 0.28 mmol) and Xantphos (0.33 g, 0.56 mmol). The reaction mixture was further purged with argon for 10 min and heated at 100° C. for 2 h. After completion, the reaction mixture was filtered through a celite bed, the filter pad washed with ethyl acetate (500 mL) and the filtrate concentrated in vacuo. The crude material was purified by combi-flash chromatography, by eluting with 90% ethyl acetate in heptane, to afford tert-butyl (R)-6-(1-((1-(tert-butoxycarbonyl)piperidin-4-yl)methyl)-3-methyl-1H-indazole-6-carboxamido)-2-(1-methylpyrrolidin-2-yl)-1H-pyrrolo[3,2-c]pyridine-1-carboxylate (Intermediate 362, 0.80 g, 63%) as a yellow solid. ¹H NMR (400 MHz, DMSO-d₆) δ ppm 1.37 (s, 9H), 1.42-1.47 (m, 2H), 1.70 (s, 9H), 1.72-1.86 (m, 6H), 2.31-2.35 (m, 2H), 2.36 (s, 3H), 2.37-2.40 (m, 1H), 2.52 (s, 3H), 2.64-2.70 (m, 2H), 3.89-3.97 (m, 3H), 4.31 (d, J=6.80 Hz, 2H), 6.76 (s, 1H), 7.73 (d, J=8.0 Hz, 1H), 7.80 (d, J=8.40 Hz 1H), 8.46 (s, 1H), 8.61 (s, 1H), 8.98 (s, 1H), 10.72 (br s, 1H). Mass spec m/z 672.5 [M+H]⁺.

### Step 5

### Intermediate 363: (R)-3-methyl-N-(2-(1-methylpyrrolidin-2-yl)-1H-pyrrolo[3,2-c]pyridin-6-yl)-1-(piperidin-4-ylmethyl)-1H-indazole-6-carboxamide dihydrochloride

To a cooled (0° C.) solution of tert-butyl (R)-6-(1-((1-(tert-butoxycarbonyl)piperidin-4-yl) methyl)-3-methyl-1H-indazole-6-carboxamido)-2-(1-methylpyrrolidin-2-yl)-1H-pyrrolo[3,2-c]pyridine-1-carboxylate (Intermediate 362, 0.75 g, 1.11 mmol) in 1,4-dioxane (10 mL) was added 4M hydrochloric acid in 1,4-dioxane (15 mL) and the reaction mixture was stirred at room temperature for 3 h. After completion, the reaction mixture was concentrated in vacuo to afford (R)-3-methyl-N-(2-(1-methylpyrrolidin-2-yl)-1H-pyrrolo[3,2-c]pyridin-6-yl)-1-(piperidin-4-ylmethyl)-1H-indazole-6-carboxamide dihydrochloride (Intermediate 363, 0.70 g) as an off-white solid, which was used for the next step without further purification. Mass spec m/z 472.3 [M+H]⁺.

### Step 6

### Example 104: 1-((1-(2-(2,6-dioxopiperidin-3-yl)-1,3-dioxoisoindolin-5-yl)piperidin-4-yl) methyl)-3-methyl-N-(2-((R)-1-methylpyrrolidin-2-yl)-1H-pyrrolo[3,2-c]pyridin-6-yl)-1H-indazole-6-carboxamide

To a solution of (R)-3-methyl-N-(2-(1-methylpyrrolidin-2-yl)-1H-pyrrolo[3,2-c]pyridin-6-yl)-1-(piperidin-4-ylmethyl)-1H-indazole-6-carboxamide dihydrochloride (Intermediate 363, 0.35 g, 0.74 mmol) in dimethylsulfoxide (5 mL) was added 2-(2,6-dioxopiperidin-3-yl)-5-fluoroisoindoline-1,3-dione (CAS No: 835616-61-0, 0.41 g, 1.48 mmol) and N,N-diisopropylethylamine (0.77 mL, 4.45 mmol). The reaction mixture was heated at 120° C. for 2 h under microwave conditions. After completion, the reaction mixture was quenched with ice cold water. The resultant precipitate was collected by filtration and dried in vacuo to afford the crude compound. The crude material was purified by combi-flash chromatography, by eluting with 10% ethyl acetate in heptane, to afford 1-((1-(2-(2,6-dioxopiperidin-3-yl)-1,3-dioxoisoindolin-5-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide (Example 104, 0.12 g, 23%) as a yellow solid. ¹H NMR (400 MHz, DMSO-d₆) δ ppm 1.41-1.45 (m, 2H), 1.53-1.56 (m, 2H), 1.78-1.91 (m, 3H), 1.97-2.03 (m, 1H), 2.12-2.14 (m, 1H), 2.16 (s, 3H), 2.22-2.30 (m, 2H), 2.51-2.52 (m, 1H), 2.53 (s, 3H), 2.54-2.59 (m, 1H), 2.82-2.91 (m, 3H), 3.11-3.18 (m, 1H), 3.30-3.33 (s, 1H), 4.06-4.10 (m, 2H), 4.31-4.35 (m, 2H), 5.03-5.07 (m, 1H), 6.40 (s, 1H), 7.23 (dd, J=8.80, 2.20 Hz, 1H), 7.30 (d, J=2.0 Hz, 1H), 7.64 (d, J=8.56 Hz, 1H), 7.73-7.76 (m, 1H), 7.80 (d, J=8.40 Hz, 1H), 8.24 (s, 1H), 8.50 (s, 2H), 10.50 (br s, 1H), 11.10 (br s, 1H), 11.41 (br s, 1H). Mass spec m/z 728.0 [M+H]⁺.

### Example 105 was Synthesised Following Scheme 87

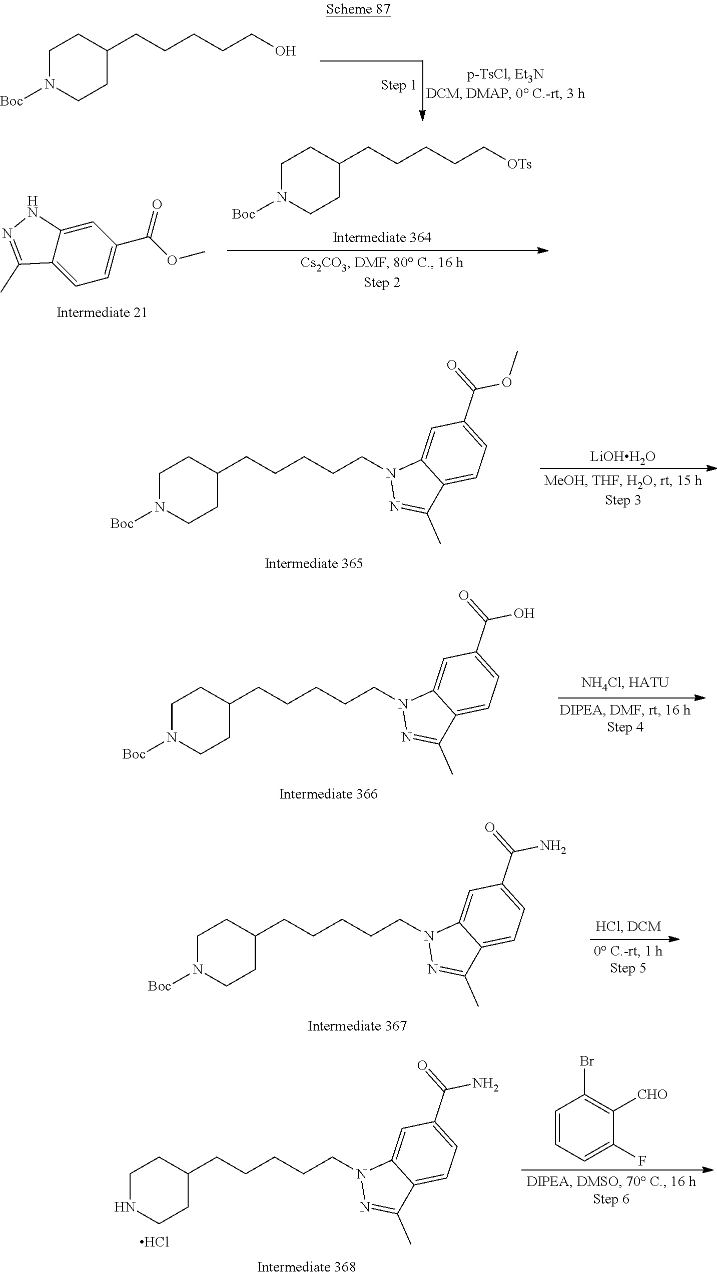

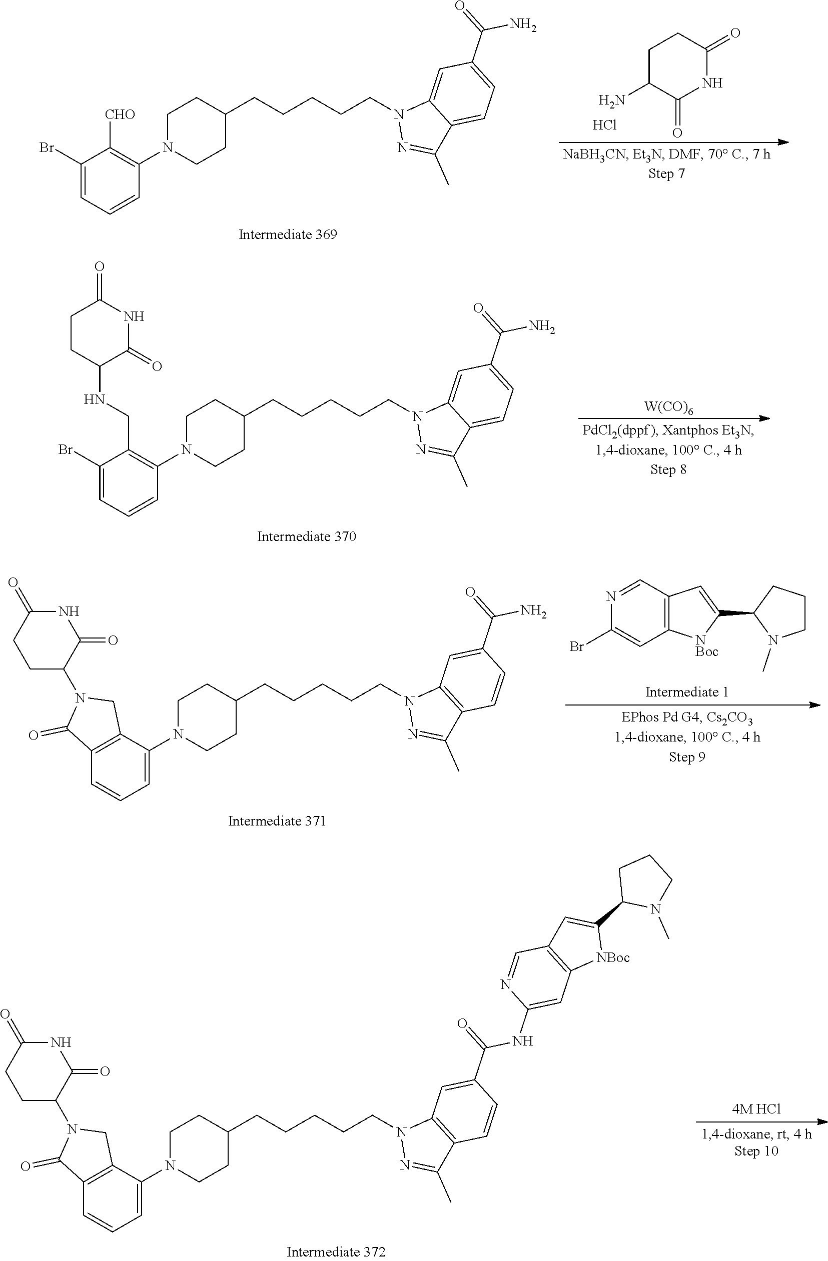

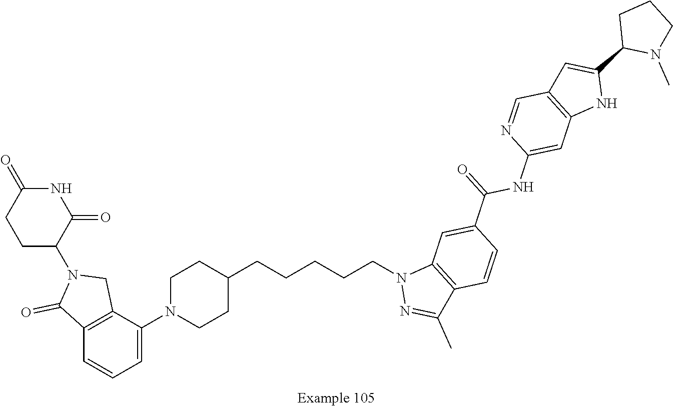 [SMILES: Cc1nn(CCCCCC2CCN(c3cccc4c3CN(C3CCC(=O)NC3=O)C4=O)CC2)c2cc(C(=O)Nc3cc4[nH]c([C@H]5CCCN5C)cc4cn3)ccc12]

### Step 1

### Intermediate 364: tert-butyl 4-(5-(tosyloxy)pentyl)piperidine-1-carboxylate

To a cooled (0° C.) solution of tert-butyl 4-(5-hydroxypentyl)piperidine-1-carboxylate (CAS No. 191725-50-5, 21.0 g, 77.4 mmol) in dichloromethane (200 mL) was added triethylamine (57 mL, 387 mmol), 4-toluenesulfonyl chloride (23.3 g, 116 mmol) and 4-dimethylaminopyridine (0.99 g, 7.73 mmol) and the reaction mixture was stirred at room temperature for 3 h. The reaction mixture was diluted with cold water (200 mL) and extracted with ethyl acetate (2×200 mL). The combined organic layers were washed with brine solution (100 mL), dried over anhydrous Na₂SO₄, filtered, and concentrated in vacuo. The crude material was purified by combi-flash chromatography, by eluting with 20-50% ethyl acetate in heptane, to afford tert-butyl 4-(5-(tosyloxy)pentyl)piperidine-1-carboxylate (Intermediate 364, 24 g, 73%) as off-white solid. ¹H NMR (400 MHz, CDCl₃) δ ppm 0.95-1.08 (m, 2H), 1.12-1.21 (m, 2H), 1.22-1.35 (m, 5H), 1.44 (s, 9H), 1.55-1.69 (m, 5H), 2.44 (s, 3H), 2.55-2.71 (m, 2H), 3.95-4.15 (m, 3H), 7.34 (d, J=8 Hz, 2H), 7.78 (d, J=8 Hz, 2H). Mass spec m/z 426.2 [M+H]⁺.

### Step 2

### Intermediate 365: methyl 1-(5-(1-(tert-butoxycarbonyl)piperidin-4-yl)pentyl)-3-methyl-1H-indazole-6-carboxylate

To a solution of methyl 3-methyl-1H-indazole-6-carboxylate (Intermediate 21, 1.0 g, 5.25 mmol) in N,N-dimethylformamide (10 mL) was added cesium carbonate (5.14 g, 15.8 mmol) followed by tert-butyl 4-(5-(tosyloxy)pentyl)piperidine-1-carboxylate (Intermediate 364, 2.68 g, 6.30 mmol) and the reaction mixture was heated at 80° C. for 16 h. The reaction mixture was diluted with cold water (100 mL) and extracted with ethyl acetate (3×100 mL). The combined organic layers were washed with brine solution (100 mL), dried over anhydrous Na₂SO₄, filtered, and concentrated in vacuo. The crude material was purified by combi-flash chromatography, by eluting with 30% ethyl acetate in heptane, to afford methyl 1-(5-(1-(tert-butoxycarbonyl)piperidin-4-yl)pentyl)-3-methyl-1H-indazole-6-carboxylate (Intermediate 365, 1.0 g, 43%) as a yellow solid. Mass spec m/z 444.3 [M+H]⁺.

### Step 3

### Intermediate 366: 1-(5-(1-(tert-butoxycarbonyl)piperidin-4-yl)pentyl)-3-methyl-1H-indazole-6-carboxylic acid

To a solution of methyl 1-(5-(1-(tert-butoxycarbonyl)piperidin-4-yl)pentyl)-3-methyl-1H-indazole-6-carboxylate (Intermediate 365, 16.0 g, 36.1 mmol) in tetrahydrofuran (50 mL), methanol (50 mL) and water (25 mL) was added lithium hydroxide (4.40 g, 180.3 mmol) and the reaction mixture was stirred at room temperature for 15 h. The reaction mixture was concentrated in vacuo, and the residue was acidified to a pH 4 by the addition of saturated aqueous citric acid solution. The resultant precipitate was collected by filtration, washed with water (20 mL) and dried in vacuo to afford 1-(5-(1-(tert-butoxycarbonyl)piperidin-4-yl)pentyl)-3-methyl-1H-indazole-6-carboxylic acid (Intermediate 366, 13 g, 84%) as a white solid which was used for the next step without further purification. ¹H NMR (400 MHz, DMSO-d₆) δ ppm 1.13-1.28 (m, 7H), 1.35 (s, 9H), 1.36-1.40 (m, 2H), 1.51-1.54 (m, 2H), 1.74-1.81 (m, 2H), 2.46 (s, 3H), 3.83-3.86 (m, 3H), 4.00-4.01 (m, 1H), 4.31-4.35 (m, 2H), 7.61-7.65 (m, 1H), 7.66-7.70 (m, 1H), 8.12 (s, 1H). Mass spec m/z 430.3 [M+H]⁺.

### Step 4

### Intermediate 367: tert-butyl 4-(5-(6-carbamoyl-3-methyl-1H-indazol-1-yl)pentyl)piperidine-1-carboxylate

To a solution of 1-(5-(1-(tert-butoxycarbonyl)piperidin-4-yl)pentyl)-3-methyl-1H-indazole-6-carboxylic acid (Intermediate 366, 12.0 g, 27.9 mmol) in N,N-dimethylformamide (50 mL) was added HATU (21.2 g, 55.9 mmol) and the reaction mixture was stirred at room temperature for 20 min. Ammonium chloride (14.9 g, 279 mmol) and N,N-diisopropylethylamine (24.4 mL, 140 mmol) were then added and the reaction mixture stirred at room temperature for 16 h. The reaction mixture was diluted with water (500 mL) and extracted with ethyl acetate (3×300 mL). The combined organic layers were dried over anhydrous Na₂SO₄, filtered, and concentrated in vacuo to afford tert-butyl 4-(5-(6-carbamoyl-3-methyl-1H-indazol-1-yl)pentyl)piperidine-1-carboxylate (Intermediate 367, 11.5 g, 96%) as a white solid, which was used for the next step without further purification. ¹H NMR (400 MHz, DMSO-d₆) δ ppm 0.82-0.98 (m, 2H), 1.12-1.26 (m, 7H), 1.37 (s, 9H), 1.52-1.57 (m, 2H), 1.76-1.86 (m, 2H), 2.49 (s, 3H), 2.59-2.62 (m, 2H), 3.83-3.87 (m, 2H), 4.31-4.33 (m, 2H), 7.43 (br s, 1H), 7.59 (d, J=8.31 Hz, 1H), 7.73 (d, J=8.31 Hz, 1H), 8.04 (s, 1H), 8.15 (br s, 1H). Mass spec m/z 429.2 [M+H]⁺.

### Step 5

### Intermediate 368: 3-methyl-1-(5-(piperidin-4-yl)pentyl)-1H-indazole-6-carboxamide hydrochloride

To a cooled (0° C.) solution of tert-butyl 4-(5-(6-carbamoyl-3-methyl-1H-indazol-1-yl)pentyl)piperidine-1-carboxylate (Intermediate 367, 7.0 g, 16.3 mmol) in 1,4 dioxane (70 mL) was added 4M hydrochloric acid in dioxane (70 mL) and the reaction mixture was stirred at room temperature for 1 h. The reaction mixture was concentrated in vacuo to afford 3-methyl-1-(5-(piperidin-4-yl)pentyl)-1H-indazole-6-carboxamide hydrochloride (Intermediate 368, 7.0 g) as an off-white solid, which was used for the next step without further purification. ¹H NMR (400 MHz, DMSO-d₆) δ ppm 1.10-1.19 (m, 3H), 1.22-1.31 (m, 8H), 1.69-1.72 (m, 2H), 1.75-1.89 (m, 2H), 3.05-3.22 (m, 2H), 2.70-2.78 (m, 2H), 4.32-4.35 (m, 2H), 7.43 (br s, 1H), 7.60 (d, J=8.31 Hz, 1H), 7.73 (d, J=8.41 Hz, 1H), 8.09 (br s, 1H), 8.16 (s, 1H), 8.73 (br s, 1H), 9.01 (br s, 1H). Mass spec m/z 329.1 [M+H]⁺.

### Step 6

### Intermediate 369: 1-(5-(1-(3-bromo-2-formylphenyl)piperidin-4-yl)pentyl)-3-methyl-1H-indazole-6-carboxamide

To a solution of 3-methyl-1-(5-(piperidin-4-yl)pentyl)-1H-indazole-6-carboxamide hydrochloride (Intermediate 368, 6.47 g, 17.7 mmol) in dimethylsulfoxide (30 mL) was added N,N-diisopropylethylamine (12.9 mL, 73.9 mmol) followed by 2-bromo-6-fluorobenzaldehyde (CAS No. 360575-28-6, 3 g, 14.8 mmol) and the reaction mixture was heated at 70° C. for 16h. The reaction mixture was diluted with water (250 mL) and extracted with ethyl acetate (2×200 mL). The combined organic layers were dried over anhydrous Na₂SO₄, filtered and concentrated in vacuo. The crude material was purified by combi-flash chromatography, by eluting with 100% ethyl acetate, to afford 1-(5-(1-(3-bromo-2-formylphenyl)piperidin-4-yl)pentyl)-3-methyl-1H-indazole-6-carboxamide (Intermediate 369, 2.5 g, 33%) as a pale yellow, which was used for the next step without further purification. ¹H NMR (400 MHz, DMSO-d₆) δ ppm 1.19-1.30 (m, 12H), 1.65-1.68 (m, 2H), 1.61-1.71 (m, 2H), 1.80-1.87 (m, 2H), 3.09-3.12 (m, 2H), 4.30-4.35 (m, 2H), 7.18 (d, J=8.07 Hz, 1H), 7.28 (d, J=8.07 Hz, 1H), 7.36-7.41 (m, 2H), 7.58 (d, J=8.44 Hz, 1H), 7.72 (d, J=8.44 Hz, 1H), 8.01 (br s, 1H), 8.11 (s, 1H), 9.98 (br s, 1H). Mass spec m/z 513.2 [M+H+2]+

### Step 7

### Intermediate 370: 1-(5-(1-(3-bromo-2-(((2,6-dioxopiperidin-3-yl)amino)methyl)phenyl)piperidin-4-yl)pentyl)-3-methyl-1H-indazole-6-carboxamide

To a solution of 1-(5-(1-(3-bromo-2-formylphenyl)piperidin-4-yl)pentyl)-3-methyl-1H-indazole-6-carboxamide (Intermediate 369, 3.0 g, 5.86 mmol) in N,N-dimethylformamide (30 mL) was added 3-aminopiperidine-2,6-dione hydrochloride (CAS No. 24666-56-6, 0.96 g, 5.86 mmol) and the reaction mixture was heated at 70° C. for 3h. Sodium cyanoborohydride (1.16 g, 17.6 mmol) was added and the reaction mixture was heated at 80° C. for 4 h. The reaction mixture was diluted with water (100 mL) and extracted with ethyl acetate (3×100 mL). The combined organic layers were dried over anhydrous Na₂SO₄, filtered and concentrated in vacuo. The crude material was purified by combi-flash chromatography, by eluting with 20% ethyl acetate in heptane, to afford 1-(5-(1-(3-bromo-2-(((2,6-dioxopiperidin-3-yl)amino)methyl)phenyl)piperidin-4-yl)pentyl)-3-methyl-1H-indazole-6-carboxamide (Intermediate 370, 2.2 g, 60%) as a yellow semi-solid. Mass spec m/z 625.1 [M+H+2]+

### Step 8

### Intermediate 371: 1-(5-(1-(2-(2,6-dioxopiperidin-3-yl)-1-oxoisoindolin-4-yl)piperidin-4-yl)pentyl)-3-methyl-1H-indazole-6-carboxamide

To a solution of 1-(5-(1-(3-bromo-2-(((2,6-dioxopiperidin-3-yl)amino)methyl)phenyl)piperidin-4-yl)pentyl)-3-methyl-1H-indazole-6-carboxamide (Intermediate 370, 1.80 g, 2.90 mmol) in 1,4-dioxane (20 mL) was added triethylamine (1.0 mL, 8.70 mmol). The reaction mixture was purged with argon for 15 min then Xantphos (0.34 g, 0.58 mmol) followed by PdCl₂(dppf) (0.44 g, 0.58 mmol) and W(CO)₆ (0.51 g, 1.40 mmol) were added. The reaction mixture was further purged with argon for 10 min and heated at 100° C. for 4 h. The reaction mixture was filtered through a celite bed, the filter pad was washed with ethyl acetate (200 mL) and the filtrate was concentrated in vacuo. The crude material was purified by combi-flash chromatography, by eluting with 80% ethyl acetate in heptane, to afford 1-(5-(1-(2-(2,6-dioxopiperidin-3-yl)-1-oxoisoindolin-4-yl)piperidin-4-yl)pentyl)-3-methyl-1H-indazole-6-carboxamide (Intermediate 371, 1.5 g, 91%) as a yellow liquid. ¹H NMR (400 MHz, DMSO-d₆) δ ppm 1.15-1.31 (m, 12H), 1.61-1.69 (m, 2H), 1.76-1.89 (m, 2H), 1.98 (s, 3H), 2.63-2.66 (m, 2H), 2.87-2.94 (m, 1H), 3.08-3.12 (m, 2H), 3.99-4.05 (m, 1H), 4.24-4.42 (m, 3H), 5.15-5.19 (m, 1H), 7.14 (d, J=8.02 Hz, 1H), 7.28 (d, J=7.24 Hz, 1H), 7.36-7.46 (m, 2H), 7.60 (d, J=7.63 Hz, 1H), 7.73 (d, J=8.61 Hz, 1H), 8.04 (br s, 1H), 8.13 (br s, 1H), 10.97 (br s, 1H). Mass spec m/z 571.4 [M+H]⁺.

### Step 9

### Intermediate 372: tert-butyl 6-(1-(5-(1-(2-(2,6-dioxopiperidin-3-yl)-1-oxoisoindolin-4-yl)piperidin-4-yl)pentyl)-3-methyl-1H-indazole-6-carboxamido)-2-((R)-1-methylpyrrolidin-2-yl)-1H-pyrrolo[3,2-c]pyridine-1-carboxylate

To a solution of 1-(5-(1-(2-(2,6-dioxopiperidin-3-yl)-1-oxoisoindolin-4-yl)piperidin-4-yl)pentyl)-3-methyl-1H-indazole-6-carboxamide (Intermediate 371, 0.7 g, 1.22 mmol) in 1,4-dioxane (7 mL) was added tert-butyl (R)-6-bromo-2-(1-methylpyrrolidin-2-yl)-1H-pyrrolo[3,2-c]pyridine-1-carboxylate (Intermediate 1, 0.46 g, 1.22 mmol) followed by cesium carbonate (1.19 g, 3.68 mmol). The reaction mixture was purged with argon for 15 min and then EPhos Pd G4 (0.17 g, 0.18 mmol) was added. The reaction mixture was further purged with argon for 10 min and heated at 100° C. for 4 h. The reaction mixture was filtered through a celite bed, the filter pad was washed with ethyl acetate (100 mL) and the filtrate concentrated in vacuo. The crude material was purified by combi-flash chromatography, by eluting with 20% ethyl acetate in heptane, to afford tert-butyl 6-(1-(5-(1-(2-(2,6-dioxopiperidin-3-yl)-1-oxoisoindolin-4-yl)piperidin-4-yl)pentyl)-3-methyl-1H-indazole-6-carboxamido)-2-((R)-1-methylpyrrolidin-2-yl)-1H-pyrrolo[3,2-c]pyridine-1-carboxylate (Intermediate 372, 0.5 g, 47%) as an off-white solid. Mass spec m/z 870.7 [M+H]⁺.

### Step 10

### Example 105: 1-(5-(1-(2-(2,6-dioxopiperidin-3-yl)-1-oxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide

To a cooled (0° C.) solution of tert-butyl 6-(1-(5-(1-(2-(2,6-dioxopiperidin-3-yl)-1-oxoisoindolin-4-yl)piperidin-4-yl)pentyl)-3-methyl-1H-indazole-6-carboxamido)-2-((R)-1-methylpyrrolidin-2-yl)-1H-pyrrolo[3,2-c]pyridine-1-carboxylate (Intermediate 372, 0.30 g, 0.34 mmol) in 1,4-dioxane (15 mL) was added 4M hydrochloric acid in 1,4-dioxane (15 mL) and the reaction mixture was stirred at room temperature for 4 h. The reaction mixture was concentrated in vacuo and the crude material was purified by preparative HPLC (Method B) to afford 1-(5-(1-(2-(2,6-dioxopiperidin-3-yl)-1-oxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide (Example 105, 0.075 g, 27%) as an off-white solid. ¹H NMR (401 MHz, DMSO-d₆) δ ppm 1.18-1.22 (m, 4H), 1.23-1.25 (s, 3H), 1.26-1.35 (m, 7H), 1.70-1.74 (m, 2H), 1.86-1.93 (m, 2H), 1.94-2.01 (m, 1H), 2.16-2.23 (m, 2H), 2.42-2.46 (m, 2H), 2.55 (s, 3H), 2.59-2.64 (m, 2H), 2.86-2.95 (m, 1H), 3.26-3.35 (m, 3H), 3.39-3.44 (m, 1H), 4.23-4.31 (m, 1H), 4.35-4.41 (m, 1H), 4.44-4.47 (m, 2H), 4.68-4.75 (m, 1H), 5.07-5.12 (m, 1H), 7.10 (d, J=7.75 Hz, 1H), 7.19 (s, 1H), 7.29 (d, J=7.13 Hz, 1H), 7.37-7.41 (m, 1H), 7.79 (d, J=8.50 Hz, 1H), 7.90 (d, J=8.51 Hz, 1H), 8.30 (s, 1H), 8.63 (s, 1H), 9.03 (s, 1H), 10.96 (br s, 1H), 11.85 (br s, 1H), 12.91 (br s, 1H). Mass spec m/z 770.5 [M+H]⁺.

### Example 106 was Synthesised Following Scheme 88

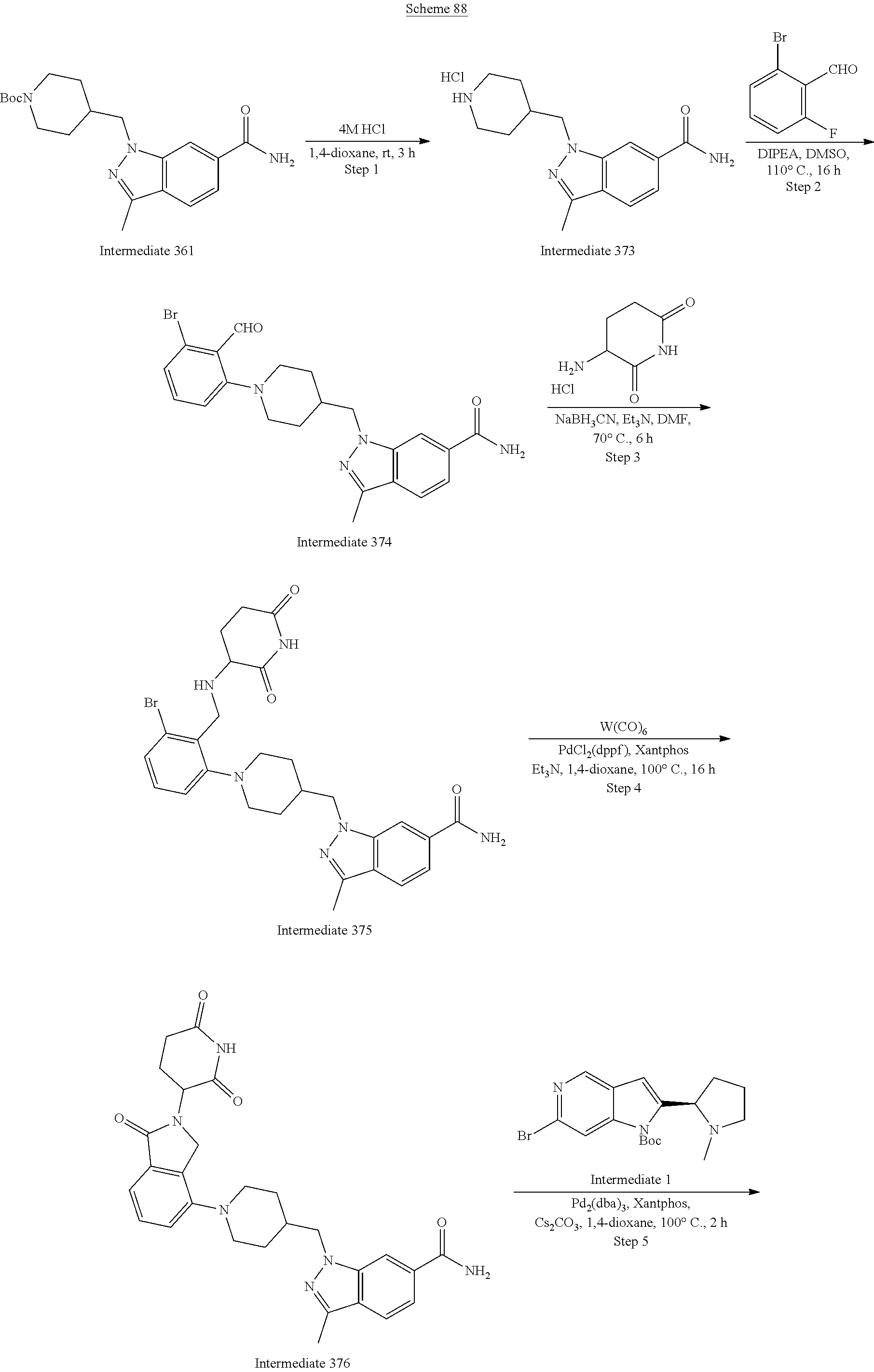

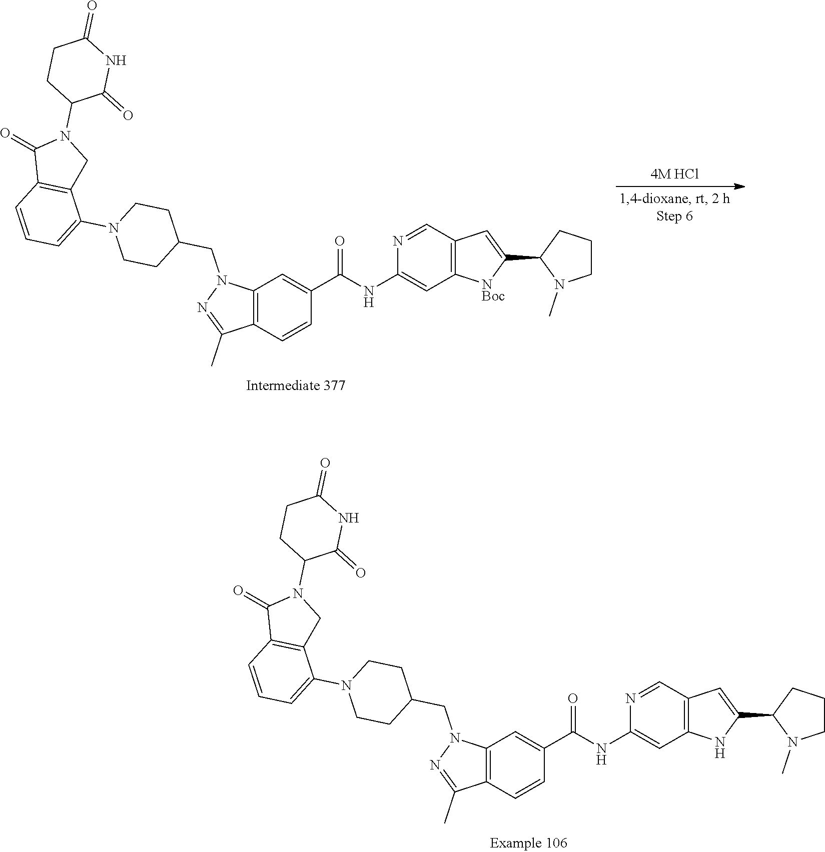 [SMILES: Cc1nn(CC2CCN(c3cccc4c3CN(C3CCC(=O)NC3=O)C4=O)CC2)c2cc(C(=O)Nc3cc4c(cn3)cc([C@H]3CCCN3C)n4C(=O)OC(C)(C)C)ccc12 ; Cc1nn(CC2CCN(c3cccc4c3CN(C3CCC(=O)NC3=O)C4=O)CC2)c2cc(C(=O)Nc3cc4[nH]c([C@H]5CCCN5C)cc4cn3)ccc12]

### Step 1

### Intermediate 373: 3-methyl-1-(piperidin-4-ylmethyl)-1H-indazole-6-carboxamide hydrochloride

To a cooled (0° C.) solution of tert-butyl 4-((6-carbamoyl-3-methyl-1H-indazol-1-yl)methyl)piperidine-1-carboxylate (Intermediate 361, 3.0 g, 8.05 mmol) in 1,4-dioxane (30 mL) was added 4M hydrochloric acid in 1,4-dioxane (30 mL). The reaction mixture was stirred at room temperature for 3 h then concentrated in vacuo to afford 3-methyl-1-(piperidin-4-ylmethyl)-1H-indazole-6-carboxamide hydrochloride (Intermediate 373, 3.0 g) as an off-white solid, which was used for the next step without further purification. ¹H NMR (400 MHz, DMSO-d₆) δ ppm 1.41-1.55 (m, 2H), 1.58-1.67 (m, 2H), 2.14-2.26 (m, 1H), 2.48 (s, 3H), 2.75-2.80 (m, 1H), 3.04-3.13 (m, 1H), 4.25-4.30 (m, 2H), 7.37-7.52 (m, 1H), 7.58-7.64 (m, 1H), 7.73 (d, J=8.61 Hz, 1H), 8.15 (br s, 1H), 8.28 (s, 1H), 8.95 (d, J=7.83 Hz, 1H), 10.55 (br s, 3H). Mass spec m/z 273.1 [M+H]⁺.

### Step 2

### Intermediate 374: 1-((1-(3-bromo-2-formylphenyl)piperidin-4-yl)methyl)-3-methyl-1H-indazole-6-carboxamide

To a solution of 3-methyl-1-(piperidin-4-ylmethyl)-1H-indazole-6-carboxamide hydrochloride (Intermediate 373, 2.90 g, 11.0 mmol) in dimethylsulfoxide (30 ml) was added N,N-diisopropylethylamine (9.30 mL, 53 mmol) followed by 2-bromo-6-fluorobenzaldehyde (CAS No. 360575-28-6, 2.20 g, 11.0 mmol) and the reaction mixture was heated at 110° C. for 16h. The reaction mixture was diluted with water (500 mL) and extracted with ethyl acetate (3×200 mL). The combined organic layers were dried over anhydrous Na₂SO₄, filtered and concentrated in vacuo. The crude material was purified by combi-flash chromatography, by eluting with 9% MeOH in DCM, to afford 1-((1-(3-bromo-2-formylphenyl)piperidin-4-yl)methyl)-3-methyl-1H-indazole-6-carboxamide (Intermediate 374, 1.7 g, 35%) as a yellow solid. ¹H NMR (400 MHz, DMSO-d₆) δ ppm 1.09-1.17 (m, 1H), 1.49-1.52 (m, 4H), 1.10-1.18 (m, 1H), 2.03 (s, 3H), 2.68-2.87 (m, 1H), 3.14-3.21 (m, 2H), 4.30-4.34 (m, 2H), 7.19 (d, J=8.12 Hz, 1H), 7.31 (d, J=7.73 Hz, 1H), 7.38-7.42 (m, 1H), 7.45 (br s, 1H), 7.61 (d, J=8.31 Hz, 1H), 7.74 (d, J=8.41 Hz, 1H), 8.04 (br s, 1H), 8.19 (s, 1H), 10.04 (br s, 1H). Mass spec m/z 455.1 [M+H]⁺.

### Step 3

### Intermediate 375: 1-((1-(3-bromo-2-(((2,6-dioxopiperidin-3-yl)amino)methyl)phenyl)piperidin-4-yl)methyl)-3-methyl-1H-indazole-6-carboxamide

To a solution of 1-((1-(3-bromo-2-formylphenyl)piperidin-4-yl)methyl)-3-methyl-1H-indazole-6-carboxamide (Intermediate 374, 1.60 g, 3.51 mmol) in N,N-dimethylformamide (20 mL) was added 3-aminopiperidine-2,6-dione hydrochloride (CAS No. 24666-56-6, 0.57 g, 3.51 mmol) and triethylamine (1.47 mL, 10.5 mmol) and the reaction mixture was heated at 70° C. for 2h. Sodium cyanoborohydride (0.93 g, 14.1 mmol) was added and the reaction mixture was heated at 70° C. for 4 h. The reaction mixture was filtered through a celite bed and the filter pad was washed with ethyl acetate (300 mL). The filtrate was concentrated in vacuo and the crude material was purified by combi-flash chromatography, by eluting with 8% MeOH in DCM, to afford 1-((1-(3-bromo-2-(((2,6-dioxopiperidin-3-yl)amino)methyl)phenyl)piperidin-4-yl)methyl)-3-methyl-1H-indazole-6-carboxamide (Intermediate 375, 1.4 g, 70%) as a yellow solid. ¹H NMR (400 MHz, DMSO-d₆) δ ppm 1.10-1.13 (m, 1H), 1.40-1.68 (m, 5H), 2.0-2.07 (m, 1H), 2.23-2.28 (m, 1H), 2.39-2.48 (m, 2H), 2.66-2.75 (m, 2H), 2.88-2.92 (m, 1H), 2.95-2.99 (m, 1H), 3.03-3.07 (m, 1H), 3.17-3.23 (m, 1H), 3.24-3.31 (m, 2H), 3.82-3.97 (m, 2H), 4.31-4.36 (m, 2H), 7.09-7.19 (m, 2H), 7.32 (dd, J=7.69, 0.94 Hz, 1H), 7.45 (br s, 1H), 7.75 (d, J=8.50 Hz, 1H), 8.05 (s, 1H), 8.20 (br s, 1H), 10.81 (br s, 1H). Mass spec m/z 569.2 [M+H+2]⁺.

### Step 4

### Intermediate 376: 1-((1-(2-(2,6-dioxopiperidin-3-yl)-1-oxoisoindolin-4-yl)piperidin-4-yl)methyl)-3-methyl-1H-indazole-6-carboxamide

To a solution of 1-((1-(3-bromo-2-(((2,6-dioxopiperidin-3-yl)amino)methyl)phenyl)piperidin-4-yl)methyl)-3-methyl-1H-indazole-6-carboxamide (Intermediate 375, 1.30 g, 2.29 mmol) in 1,4-dioxane (20 mL) was added triethylamine (0.96 mL, 6.87 mmol). The reaction mixture was purged with argon for 15 min then Xantphos (0.27 g, 0.45 mmol) followed by PdCl₂(dppf) (0.35 g, 0.45 mmol) and W(CO)₆ (0.41 g, 1.53 mmol) were added. The reaction mixture was further purged with argon for 10 min and heated at 100° C. for 16 h. The reaction mixture was filtered through a celite bed and the filter pad was washed with ethyl acetate (100 mL). The filtrate was concentrated in vacuo and the crude material was purified by combi-flash chromatography, by eluting with 8% MeOH in DCM, to afford 1-((1-(2-(2,6-dioxopiperidin-3-yl)-1-oxoisoindolin-4-yl)piperidin-4-yl)methyl)-3-methyl-1H-indazole-6-carboxamide (Intermediate 376, 0.70 g, 59%) as a yellow solid. ¹H NMR (400 MHz, DMSO-d₆), δ ppm 1.15-1.19 (m, 2H), 1.55 (s, 3H), 1.97-2.07 (m, 2H), 2.64-2.72 (m, 2H), 2.90-2.97 (m, 2H), 3.05-3.06 (m, 1H), 3.06-3.15 (m, 2H), 3.35-3.44 (m, 2H), 4.25-4.44 (m, 4H), 5.07-5.10 (m, 1H), 7.14 (d, J=7.83 Hz, 1H), 7.29 (d, J=7.43 Hz, 1H), 7.38-7.43 (m, 1H), 7.45 (br s, 1H), 7.60 (d, J=8.31 Hz, 1H), 7.75 (d, J=8.31 Hz, 1H), 8.04 (s, 1H), 8.19 (br s, 1H), 10.96 (br s, 1H). Mass spec m/z 515.3 [M+H]⁺.

### Step 5

### Intermediate 377: tert-butyl 6-(1-((1-(2-(2,6-dioxopiperidin-3-yl)-1-oxoisoindolin-4-yl)piperidin-4-yl)methyl)-3-methyl-1H-indazole-6-carboxamido)-2-((R)-1-methylpyrrolidin-2-yl)-1H-pyrrolo[3,2-c]pyridine-1-carboxylate

To a solution of 1-((1-(2-(2,6-dioxopiperidin-3-yl)-1-oxoisoindolin-4-yl)pipridin-4-yl)methyl)-3-methyl-1H-indazole-6-carboxamide (Intermediate 376, 0.50 g, 0.97 mmol) in 1,4-dioxane (30 mL) was added tert-butyl (R)-6-bromo-2-(1-methylpyrrolidin-2-yl)-1H-pyrrolo[3,2-c]pyridine-1-carboxylate (Intermediate 1, 0.44 g, 1.16 mmol) followed by cesium carbonate (0.94 g, 2.91 mmol). The reaction mixture was purged with argon for 15 min, then Pd₂(dba)₃ (0.13 g, 0.14 mmol) and Xantphos (0.16 g, 0.29 mmol) were added. The reaction mixture was further purged with argon for another 10 min and heated at 100° C. for 2 h. The reaction mixture was filtered through a celite bed and the filter pad was washed with ethyl acetate (300 mL). The filtrate was concentrated in vacuo and the crude material was purified by combi-flash chromatography, by eluting with 9% MeOH in DCM, to afford tert-butyl 6-(1-((1-(2-(2,6-dioxopiperidin-3-yl)-1-oxoisoindolin-4-yl)piperidin-4-yl)methyl)-3-methyl-1H-indazole-6-carboxamido)-2-((R)-1-methylpyrrolidin-2-yl)-1H-pyrrolo[3,2-c]pyridine-1-carboxylate (Intermediate 377, 0.40 g, 51%) as a yellow solid. ¹H NMR (400 MHz, DMSO-d₆) δ ppm 1.39-1.53 (m, 5H), 1.55-1.66 (m, 6H), 1.71 (s, 9H), 1.91-1.98 (m, 2H), 2.36 (s, 3H), 2.54 (s, 3H), 2.63-2.75 (m, 3H), 3.07-3.18 (m, 2H), 3.36-3.44 (m, 2H), 3.89-3.93 (m, 1H), 4.01-4.04 (m, 1H), 4.25-4.43 (m, 3H), 6.77 (s, 2H), 7.15 (d, J=7.75 Hz, 1H), 7.28 (d, J=7.00 Hz, 1H), 7.39-7.44 (m, 1H), 7.72-7.76 (m, 1H), 7.78-7.83 (m, 1H), 8.51 (s, 1H), 8.62 (s, 1H), 10.76 (br s, 1H), 10.95 (br s, 1H). Mass spec m/z 814.5 [M+H]⁺.

### Step 6

### Example 106: 1-((1-(2-(2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide

To a cooled (0° C.) solution of tert-butyl 6-(1-((1-(2-(2,6-dioxopiperidin-3-yl)-1-oxoisoindolin-4-yl)piperidin-4-yl)methyl)-3-methyl-1H-indazole-6-carboxamido)-2-((R)-1-methylpyrrolidin-2-yl)-1H-pyrrolo[3,2-c]pyridine-1-carboxylate (Intermediate 377, 0.40 g, 0.49 mmol) in 1,4-dioxane (10 mL) was added 4M hydrochloric acid in 1,4-dioxane (10 mL). The reaction mixture was stirred at room temperature for 2 h then concentrated in vacuo. The crude material was purified by preparative HPLC (Method A) to afford 1-((1-(2-(2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide (Example 106, 0.12 g, 35%) as an off-white solid. ¹H NMR (400 MHz, DMSO-d₆) δ ppm 1.45-1.64 (m, 4H), 1.76-1.99 (m, 5H), 2.10-2.17 (m, 2H), 2.17 (s, 3H), 2.22-2.30 (m, 1H), 2.40-2.46 (m, 2H), 2.54 (s, 3H), 2.56-2.60 (m, 1H), 2.70-2.76 (m, 1H), 2.83-2.94 (m, 1H), 3.12-3.18 (m, 1H), 3.34-3.44 (m, 2H), 4.24-4.32 (m, 1H), 4.35-4.43 (m, 3H), 5.06-5.11 (m, 1H), 6.40 (s, 1H), 7.15 (d, J=7.75 Hz, 1H), 7.28 (d, J=7.00 Hz, 1H), 7.39-7.44 (m, 1H), 7.72-7.76 (m, 1H), 7.78-7.83 (m, 1H), 8.25 (s, 1H), 8.51 (s, 1H), 8.52 (s, 1H), 10.52 (br s, 1H), 10.94 (br s, 1H), 11.40 (br s, 1H). Mass spec m/z 714.2 [M+H]⁺.

### Examples 107 and 108 were Prepared Following Scheme 89

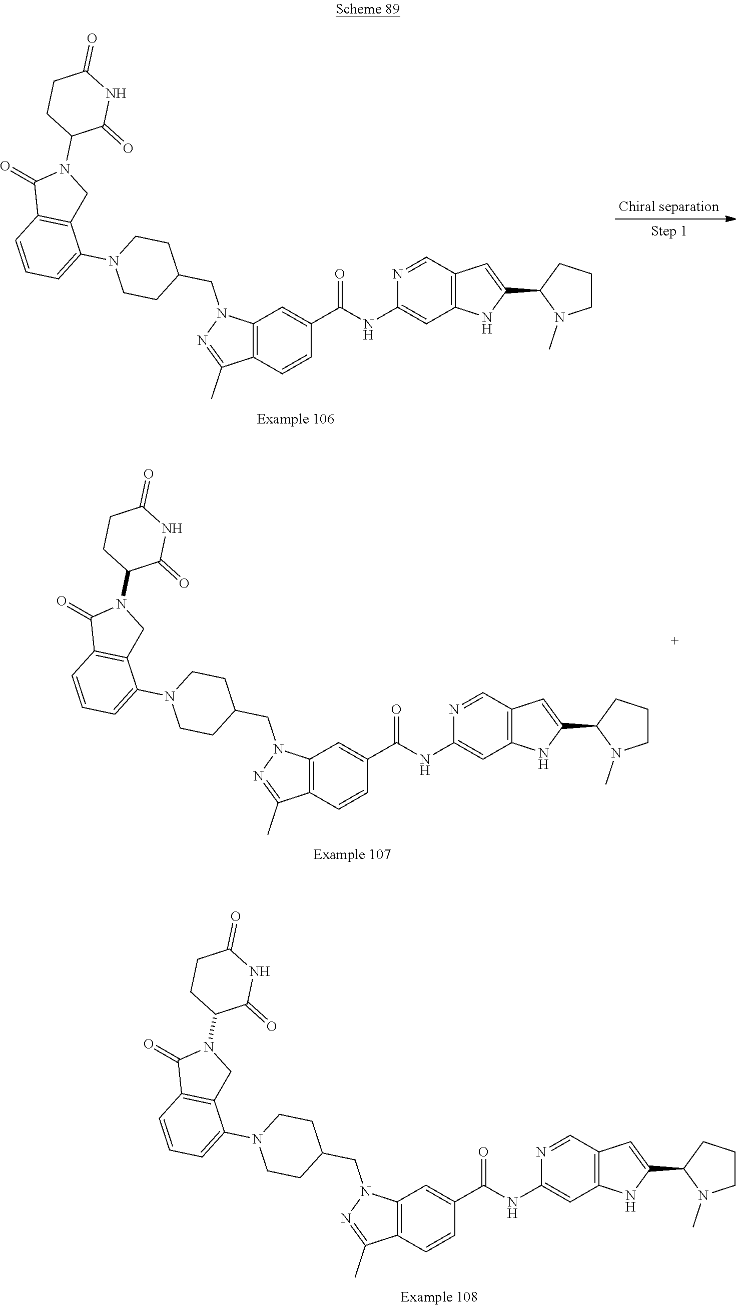

### Step 1

### Example 107: 1-((1-(2-((rel-S)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide

### and Example 108: 1-((1-(2-((rel-R)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide

1-((1-(2-(2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide (Example 106, 0.06 g, 0.08 mmol) was separated by chiral preparative HPLC (Column: RR WHELK (30×250*mm), 5 m; Mobile Phase A: DCM, Mobile Phase B: iPrOH (8:2); Flow rate: 56 ml/min; isocratic 60% B) to afford 1-((1-(2-((rel-S)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide (Example 107, 1st eluting peak, 0.018 g, 30%) as an off-white solid and 1-((1-(2-((rel-R)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide (Example 108, 2nd eluting peak, 0.017 g, 28%) as an off-white solid.

### Example 107: 1-((1-(2-((rel-S)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide

1^{st} eluting peak from Chiral Preparative HPLC. Retention time: 7.50 min.

¹H NMR (400 MHz, DMSO-d₆) δ ppm 1.45-1.54 (m, 2H), 1.56-1.64 (m, 2H), 1.80-2.01 (m, 4H), 2.10-2.25 (m, 4H), 2.43-2.47 (m, 2H), 2.54 (s, 3H), 2.56-2.59 (m, 1H), 2.66-2.68 (m, 1H), 2.70-2.73 (m, 1H), 2.84-2.94 (m, 1H), 3.29-3.41 (m, 5H), 4.26-4.45 (m, 4H), 5.06-5.11 (m, 1H), 6.43 (br s, 1H), 7.15 (d, J=7.75 Hz, 1H), 7.29 (d, J=7.00 Hz, 1H), 7.38-7.44 (m, 1H), 7.73-7.78 (m, 1H), 7.78-7.83 (m, 1H), 8.27 (s, 1H), 8.52 (s, 1H), 8.55 (s, 1H), 10.53 (br s, 1H), 10.94 (br s, 1H), 11.41 (br s, 1H). Mass spec m/z 714.5 [M+H]⁺.

### Example 108: 1-((1-(2-((rel-R)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide

2nd eluting peak from Chiral Preparative HPLC. Retention time: 9.77 min.

¹H NMR (400 MHz, DMSO-d₆) δ ppm 1.43-1.54 (m, 2H), 1.56-1.64 (m, 2H), 1.90-2.06 (m, 3H), 2.16 (s, 3H), 2.18-2.25 (m, 1H), 2.43-2.45 (m, 1H), 2.54 (s, 3H), 2.56-2.61 (m, 2H), 2.65-2.68 (m, 2H), 2.84-2.95 (m, 2H), 3.34-3.44 (m, 5H), 4.25-4.44 (m, 4H), 5.07-5.11 (m, 1H), 6.43 (br s, 1H), 7.15 (d, J=7.88 Hz, 1H), 7.29 (d, J=7.13 Hz, 1H), 7.38-7.45 (m, 1H), 7.72-7.77 (m, 1H), 7.79-7.84 (m, 1H), 8.29 (s, 1H), 8.51 (s, 1H), 8.58 (s, 1H), 10.56 (br s, 1H), 10.94 (br s, 1H), 11.44 (br s, 1H). Mass spec m/z 714.3 [M+H]⁺.

### Example 109 was Synthesised Following Scheme 90

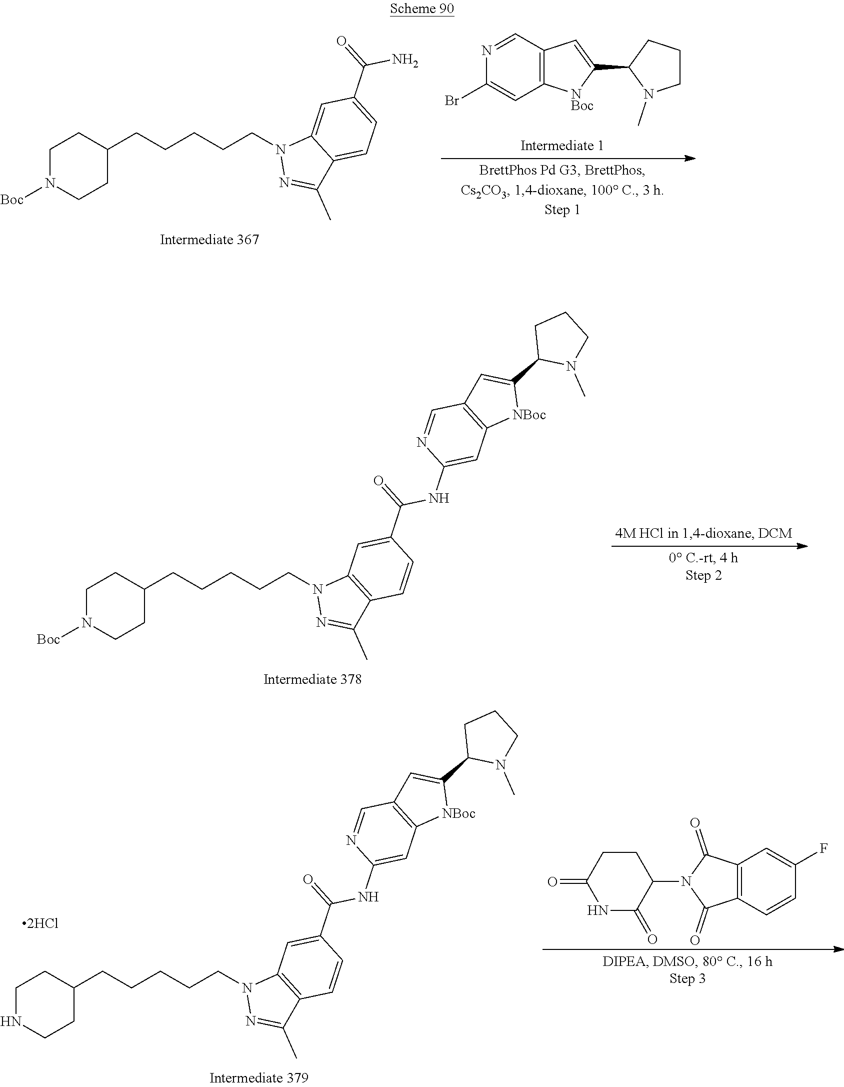 [SMILES: Cc1nn(CCCCCC2CCN(C(=O)OC(C)(C)C)CC2)c2cc(C(N)=O)ccc12 ; CN1CCC[C@@H]1c1cc2cnc(Br)cc2n1C(=O)OC(C)(C)C ; Cc1nn(CCCCCC2CCN(C(=O)OC(C)(C)C)CC2)c2cc(C(=O)Nc3cc4c(cn3)cc([C@H]3CCCN3C)n4C(=O)OC(C)(C)C)ccc12 ; Cc1nn(CCCCCC2CCNCC2)c2cc(C(=O)Nc3cc4c(cn3)cc([C@H]3CCCN3C)n4C(=O)OC(C)(C)C)ccc12 ; O=C1CCC(N2C(=O)c3ccc(F)cc3C2=O)C(=O)N1]

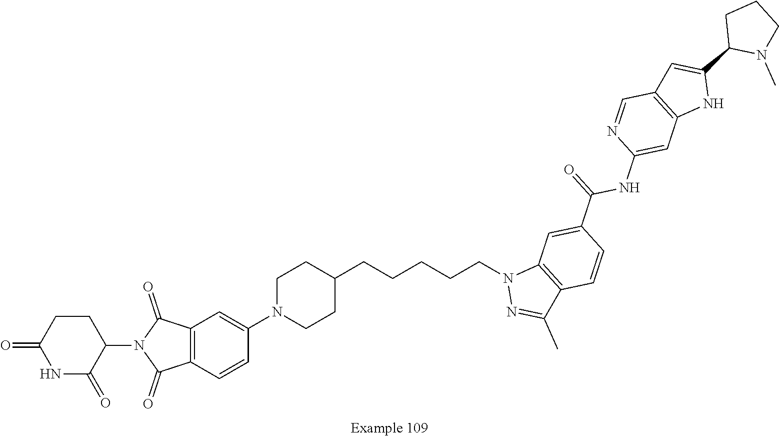 [SMILES: Cc1nn(CCCCCC2CCN(c3ccc4c(c3)C(=O)N(C3CCC(=O)NC3=O)C4=O)CC2)c2cc(C(=O)Nc3cc4[nH]c([C@H]5CCCN5C)cc4cn3)ccc12]

### Step 1

### Intermediate 378: tert-butyl (R)-6-(1-(5-(1-(tert-butoxycarbonyl)piperidin-4-yl)pentyl)-3-methyl-1H-indazole-6-carboxamido)-2-(1-methylpyrrolidin-2-yl)-1H-pyrrolo[3,2-c]pyridine-1-carboxylate

To a solution of tert-butyl 4-(5-(6-carbamoyl-3-methyl-1H-indazol-1-yl)pentyl)piperidine-1-carboxylate (Intermediate 367, 1.18 g, 2.76 mmol) in 1,4-dioxane (10 mL) was added tert-butyl (R)-6-bromo-2-(1-methylpyrrolidin-2-yl)-1H-pyrrolo[3,2-c]pyridine-1-carboxylate (Intermediate 1, 1.0 g, 2.63 mmol) followed by cesium carbonate (2.57 g, 7.88 mmol). The reaction mixture was purged with argon for 15 min then BrettPhos Pd G3 (0.97 g, 1.05 mmol) and BrettPhos (0.43 g, 0.78 mmol) were added. The reaction mixture was further purged with argon for 10 min and heated at 100° C. for 3 h. The reaction mixture was filtered through a celite bed, and the filter pad was washed with ethyl acetate (100 mL). The filtrate was concentrated in vacuo and the crude material was purified by combi-flash chromatography, by eluting with 10% MeOH in DCM, to afford tert-butyl (R)-6-(1-(5-(1-(tert-butoxycarbonyl)piperidin-4-yl)pentyl)-3-methyl-1H-indazole-6-carboxamido)-2-(1-methylpyrrolidin-2-yl)-1H-pyrrolo[3,2-c]pyridine-1-carboxylate (Intermediate 378, 1.1 g, 25%) as an off-white solid. Mass spec m/z 628.5 [M-100+H]⁺.

### Step 2

### Intermediate 379: (R)-3-methyl-N-(2-(1-methylpyrrolidin-2-yl)-1H-pyrrolo[3,2-c]pyridin-6-yl)-1-(5-(piperidin-4-yl)pentyl)-1H-indazole-6-carboxamide dihydrochloride

To a cooled (0° C.) solution of tert-butyl (R)-6-(1-(5-(1-(tert-butoxycarbonyl)piperidin-4-yl)pentyl)-3-methyl-1H-indazole-6-carboxamido)-2-(1-methylpyrrolidin-2-yl)-1H-pyrrolo[3,2-c]pyridine-1-carboxylate (Intermediate 378, 2.50 g, 3.40 mmol) in 1,4-dioxane (25 mL) was added 4M hydrochloric acid in dioxane (25 mL) and the reaction mixture was stirred at room temperature for 4 h. The reaction mixture was concentrated in vacuo to afford (R)-3-methyl-N-(2-(1-methylpyrrolidin-2-yl)-1H-pyrrolo[3,2-c]pyridin-6-yl)-1-(5-(piperidin-4-yl)pentyl)-1H-indazole-6-carboxamide dihydrochloride (Intermediate 379, 1.0 g, 55%) as an off-white solid, which was used for the next step without further purification. ¹H NMR (400 MHz, DMSO-d₆) δ ppm 1.15-1.28 (m, 10H), 1.38-1.50 (m, 3H), 1.68-1.77 (m, 2H), 1.86-1.91 (m, 2H), 2.14-2.25 (m, 2H), 2.55 (s, 3H), 2.75-2.79 (m, 2H), 2.82 (s, 3H), 3.12-3.16 (m, 1H), 3.69-3.79 (m, 2H), 4.44-4.47 (m, 2H), 7.29 (s, 1H), 7.82-7.91 (m, 2H), 8.43 (s, 1H), 8.81 (s, 2H), 9.13 (s, 1H). Mass spec m/z 528.7 [M+H]⁺.

### Step 3

### Example 109: 1-(5-(1-(2-(2,6-dioxopiperidin-3-yl)-1,3-dioxoisoindolin-5-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide

To a solution of (R)-3-methyl-N-(2-(1-methylpyrrolidin-2-yl)-1H-pyrrolo[3,2-c]pyridin-6-yl)-1-(5-(piperidin-4-yl)pentyl)-1H-indazole-6-carboxamide dihydrochloride (Intermediate 379, 0.60 g, 1.13 mmol) in dimethylsulfoxide (6 mL) was added N,N-diisopropylethylamine (0.99 mL, 5.68 mmol) followed by 2-(2,6-dioxopiperidin-3-yl)-5-fluoroisoindoline-1,3-dione (CAS No. 835616-61-0, 0.31 g, 1.13 mmol) and the reaction mixture was heated at 80° C. for 16 h. The reaction mixture was poured into ice cold water (30 mL), the resultant precipitate was collected by filtration and dried in vacuo. The crude material was purified by preparative HPLC (Method A) to afford 1-(5-(1-(2-(2,6-dioxopiperidin-3-yl)-1,3-dioxoisoindolin-5-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide (Example 109, 0.068 g, 8%) as an off-white solid. ¹H NMR (400 MHz, DMSO-d₆) δ ppm 1.10-1.18 (m, 5H), 1.20-1.29 (m, 2H), 1.31-1.35 (m, 2H), 1.40-1.49 (m, 1H), 1.68-1.71 (m, 2H), 1.76-1.93 (m, 5H), 1.95-2.05 (m, 1H), 2.10-2.16 (m, 1H), 2.16 (s, 3H), 2.21-2.29 (m, 1H), 2.53 (s, 3H), 2.55-2.62 (m, 2H), 2.81-2.90 (m, 3H), 3.12-3.16 (m, 1H), 3.94-3.96 (m, 2H), 4.39-4.43 (m, 2H), 5.02-5.07 (m, 1H), 6.40 (s, 1H), 7.10 (dd, J=8.75, 2.13 Hz, 1H), 7.24 (s, 1H), 7.58 (d, J=8.63 Hz, 1H), 7.72-7.79 (m, 2H), 8.26 (s, 1H), 8.46 (s, 1H), 8.52 (s, 1H), 10.55 (br s, 1H), 11.06 (br s, 1H), 11.39 (br s, 1H). Mass spec m/z 784.4 [M+H]⁺.

### Example 110 was Synthesised Following Scheme 90

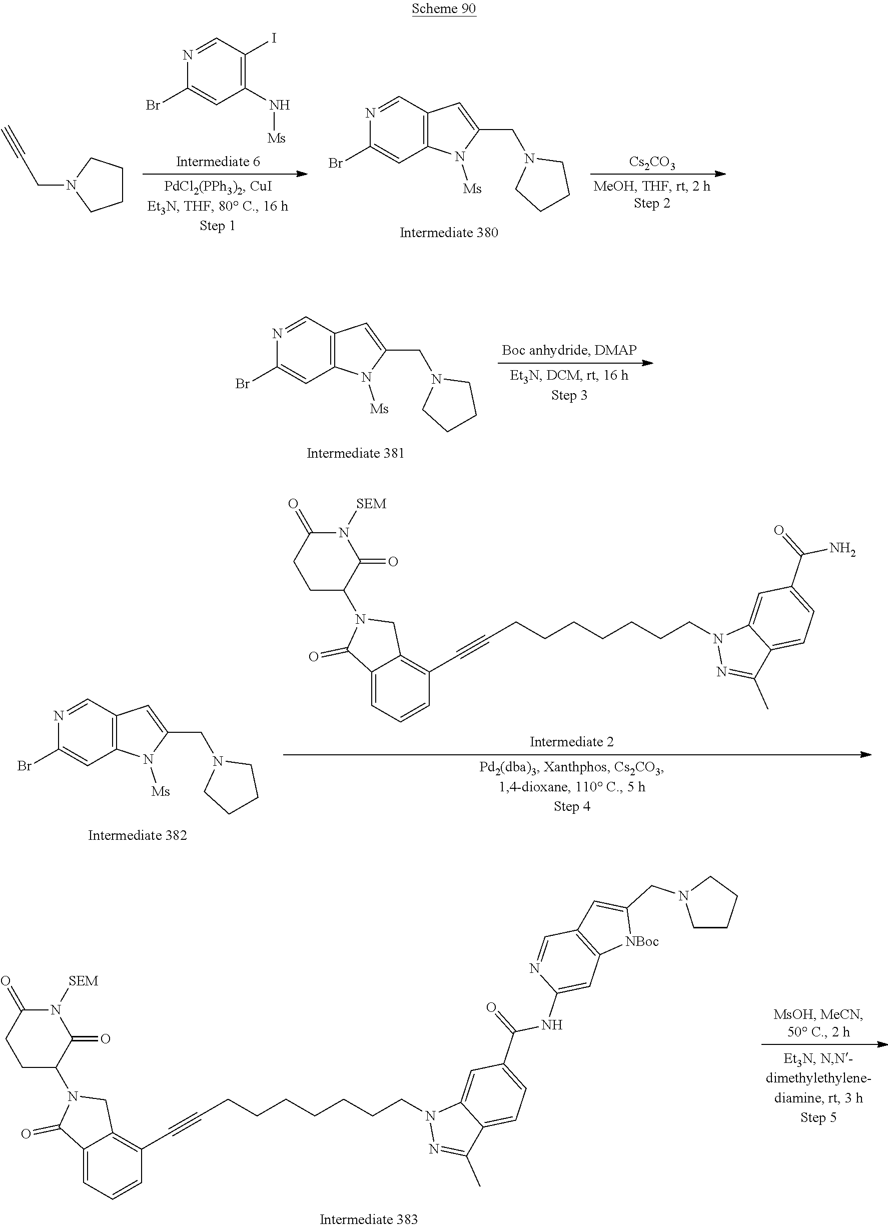

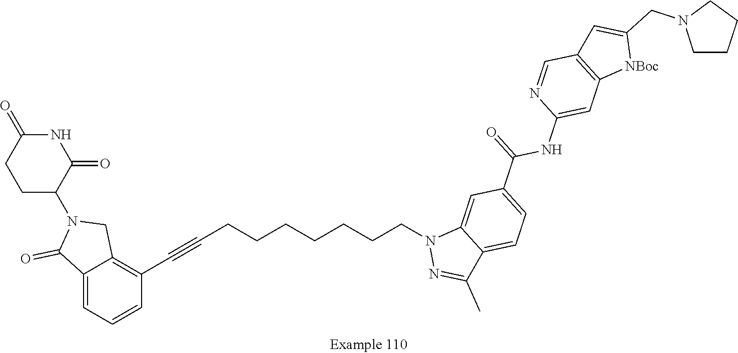 [SMILES: Cc1nn(CCCCCCCC#Cc2cccc3c2CN(C2CCC(=O)NC2=O)C3=O)c2cc(C(=O)Nc3cc4c(cn3)cc(CN3CCCC3)n4C(=O)OC(C)(C)C)ccc12]

### Step 1

### Intermediate 380: 6-bromo-1-(methylsulfonyl)-2-(pyrrolidin-1-ylmethyl)-1H-pyrrolo[3,2-c]pyridine

To a solution of 1-(prop-2-yn-1-yl)pyrrolidine (CAS No. 7223-42-9, 10.0 g, 26.52 mmol) in tetrahydrofuran (100 mL) was added N-(2-bromo-5-iodopyridin-4-yl)methanesulfonamide (Intermediate 6, 4.34 g, 39.8 mmol) followed by N,N-diisopropylethylamine (37.1 mL, 212 mmol). The reaction mixture was purged with argon for 15 min then copper(I) iodide (1.01 g, 5.30 mmol) and PdCl₂(PPh₃)₂ (3.84 g, 5.30 mmol) were added. The reaction mixture was purged with argon for another 10 min and stirred at 80° C. for 16 h. The reaction mixture was concentrated in vacuo and the crude material was purified by combi-flash column chromatography, by eluting with 100% ethyl acetate, to afford 6-bromo-1-(methylsulfonyl)-2-(pyrrolidin-1-ylmethyl)-1H-pyrrolo[3,2-c]pyridine (Intermediate 380, 5.8 g, 61%) as a brown solid. ¹H NMR (400 MHz, DMSO-d₆) δ ppm 1.67-1.73 (m, 4H), 2.49-2.51 (m, 4H), 3.77 (s, 3H), 3.90 (s, 2H), 6.88 (s, 1H), 7.99 (s, 1H), 8.69 (s, 1H). Mass spec m/z 359.5 [M+H]⁺.

### Step 2

### Intermediate 381: 6-bromo-2-(pyrrolidin-1-ylmethyl)-1H-pyrrolo[3,2-c]pyridine

To a cooled (0° C.) solution of 6-bromo-1-(methylsulfonyl)-2-(pyrrolidin-1-ylmethyl)-1H-pyrrolo[3,2-c]pyridine (Intermediate 380, 7.8 g, 22 mmol) in methanol (80 mL) and tetrahydrofuran (80 mL) was added cesium carbonate (11 g, 33 mmol) and the reaction mixture was stirred at room temperature for 2 h. The reaction mixture was quenched with water (2000 mL) and extracted with ethyl acetate (2×1000 mL). The combined organic layers were dried over anhydrous Na₂SO₄ and concentrated in vacuo to afford 6-bromo-2-(pyrrolidin-1-ylmethyl)-1H-pyrrolo[3,2-c]pyridine (Intermediate 381, 4.1 g) as a yellow solid, which was used for the next step without further purification. ¹H NMR (400 MHz, DMSO-d₆) δ ppm 1.68-1.74 (m, 4H), 2.43-2.48 (m, 4H), 3.72 (s, 2H), 6.43 (s, 1H), 7.44 (s, 1H), 8.50 (s, 1H), 11.63 (br s, 1H). Mass spec m/z 279.8 [M+H]⁺.

### Step 3

### Intermediate 382: tert-butyl 6-bromo-2-(pyrrolidin-1-ylmethyl)-1H-pyrrolo[3,2-c]pyridine-1-carboxylate

To a solution of 6-bromo-2-(pyrrolidin-1-ylmethyl)-1H-pyrrolo[3,2-c]pyridine (Intermediate 381, 4.1 g, 15 mmol) in dichloromethane (40 mL) was added triethylamine (6 mL, 44.0 mmol) followed by di-tert-butyl dicarbonate (6.8 mL, 29.0 mmol) and 4-(dimethylamino)pyridine (0.18 g, 1.5 mmol) and the reaction mixture was stirred at room temperature for 16 h. The reaction mixture was quenched with water (500 mL) and extracted with ethyl acetate (3×500 mL). The combined organic layers were dried over anhydrous Na₂SO₄ and concentrated in vacuo. The crude material was purified by combi-flash chromatography, eluting with 100% ethyl acetate, to afford tert-butyl 6-bromo-2-(pyrrolidin-1-ylmethyl)-1H-pyrrolo[3,2-c]pyridine-1-carboxylate (Intermediate 382, 2.8 g, 50%) as an off-white solid. ¹H NMR (400 MHz, DMSO-d₆) δ ppm 1.63 (s, 9H), 1.68-1.74 (m, 4H), 2.51-2.56 (m, 4H), 3.92 (s, 2H), 6.77 (s, 1H), 8.02 (s, 1H), 8.62 (s, 1H). Mass spec m/z 379.9 [M+H]⁺.

### Step 4

### Intermediate 383: tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-(pyrrolidin-1-ylmethyl)-1H-pyrrolo[3,2-c]pyridine-1-carboxylate

To a solution of tert-butyl 6-bromo-2-(pyrrolidin-1-ylmethyl)-1H-pyrrolo[3,2-c]pyridine-1-carboxylate (Intermediate 382, 0.34 g, 0.89 mmol) in 1,4-dioxane (5.0 mL) was added 1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamide (Intermediate 2, 0.5 g, 0.74 mmol) followed by cesium carbonate (0.73 g, 2.23 mmol). The reaction mixture was purged with argon for 15 min then Xantphos (0.13 g, 0.22 mmol) and Pd₂(dba)₃ (0.10 g, 0.11 mmol) were added. The reaction mixture was purged with argon for another 10 min and heated at 110° C. for 5 h. The reaction mixture was concentrated in vacuo and the crude material was purified by combi-flash chromatography, by eluting with 100% ethyl acetate, to afford tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-(pyrrolidin-1-ylmethyl)-1H-pyrrolo[3,2-c]pyridine-1-carboxylate (Intermediate 383, 0.60 g, 83%) as an off-white solid. ¹H NMR (400 MHz, DMSO-d₆) δ ppm −0.06 (s, 9H), 0.76-0.88 (m, 4H), 1.24-1.36 (m, 8H), 1.49-1.57 (m, 2H), 1.67 (s, 9H), 1.72 (s, 3H), 1.81-1.90 (m, 2H), 2.01-2.05 (m, 1H), 2.37-2.44 (m, 2H), 2.51-2.57 (m, 3H), 2.71-2.83 (m, 2H), 2.99-3.10 (m, 2H), 3.47-3.52 (m, 2H), 3.94 (s, 2H), 4.21-4.25 (m, 1H), 4.39-4.45 (m, 3H), 4.96-5.07 (m, 2H), 5.24-5.30 (m, 1H), 6.72 (s, 1H), 7.44-7.52 (m, 1H), 7.60 (d, J=7.46 Hz, 1H), 7.67-7.74 (m, 2H), 7.76-7.83 (m, 1H), 8.43 (s, 1H), 8.61 (s, 1H), 8.94 (s, 1H), 10.77 (s, 1H). Mass spec m/z 969.4 [M+H]⁺.

### Step 5

### Example 110: 1-(9-(2-(2,6-dioxopiperidin-3-yl)-1-oxoisoindolin-4-yl)non-8-yn-1-yl)-3-methyl-N-(2-(pyrrolidin-1-ylmethyl)-1H-pyrrolo[3,2-c]pyridin-6-yl)-1H-indazole-6-carboxamide

To a solution of tert-butyl 6-(1-(9-(2-(2,6-dioxo-1-((2-(trimethylsilyl)ethoxy)methyl)piperidin-3-yl)-1-oxoisoindolin-4-yl)non-8-yn-1-yl)-3-methyl-1H-indazole-6-carboxamido)-2-(pyrrolidin-1-ylmethyl)-1H-pyrrolo[3,2-c]pyridine-1-carboxylate (Intermediate 383, 0.6 g, 0.67 mmol) in acetonitrile (7 mL) was added methanesulfonic acid (0.44 mL, 6.79 mmol) and the reaction mixture was heated at 50° C. for 2 h. The reaction mixture was cooled to room temperature, then N,N′-dimethylethylenediamine (0.40 mL, 0.33 mmol) and triethylamine (1.90 mL, 13.6 mmol) were added, and the reaction mixture was stirred at room temperature for 3 h. The reaction mixture was concentrated in vacuo and the resultant residue was diluted with water (100 mL). The resultant precipitate was collected by filtration and dried in vacuo. The crude material was purified by preparative HPLC (Method A) to afford 1-(9-(2-(2,6-dioxopiperidin-3-yl)-1-oxoisoindolin-4-yl)non-8-yn-1-yl)-3-methyl-N-(2-(pyrrolidin-1-ylmethyl)-1H-pyrrolo[3,2-c]pyridin-6-yl)-1H-indazole-6-carboxamide (Example 110, 0.17 g, 34%) as an off-white solid. ¹H NMR (401 MHz, DMSO-d₆) δ ppm 1.26-1.38 (m, 7H), 1.45-1.59 (m, 2H), 1.73 (s, 3H), 1.73-1.76 (m, 1H), 1.85-1.88 (m, 2H), 1.98-2.03 (m, 1H), 2.37-2.46 (m, 5H), 2.52-2.52 (m, 3H), 2.55-2.57 (m, 2H), 2.83-2.94 (m, 1H), 3.71 (s, 2H), 4.26-4.44 (m, 4H), 5.10-5.14 (m, 1H), 6.37 (s, 1H), 7.44-7.50 (m, 1H), 7.59 (d, J=7.13 Hz, 1H), 7.67-7.75 (m, 2H), 7.75-7.80 (m, 1H), 8.24 (s, 1H), 8.43 (s, 1H), 8.52 (s, 1H), 10.53 (br s, 1H), 11.00 (br s, 1H), 11.46 (br s, 1H). Mass spec m/z 739.4 [M+H]⁺.

### Example 111: Efficacy in Vitro

### MLLT1 Degradation Assay:

MLLT1 degradation efficacy of compounds was tested in an automated chemiluminescent detection system (JESS, Cat #004-650). MV4-11 (#CRL-9591, ATCC) cells were seeded at a density of 1.2 million cells/well in a non-treated 6-well cell culture plate in 2.4 mL of complete medium (IMDM+10% FBS+1× Penicillin-Streptomycin). 10 mM DMSO stocks were prepared, and cells were treated with three concentrations of test compounds viz. 1000, 100 and 10 nM at a final DMSO concentration of 0.1% and incubated for 24 h at 37° C., 5% CO₂. Post incubation, cells were centrifuged, washed with HBSS and lysed in 70 μL of RIPA buffer supplemented with Protease inhibitors (sc-24948; Santa Cruz). Subsequently, protein estimation was done using BCA protein assay kit (23225; Pierce). 4 ug protein lysate (4 uL of 1 ug/uL lysate) was loaded into each well of the JESS cartridge (SM-W001; Bio-techne) and the JESS run was performed using 1:900 dilution of anti-human MLLT1 antibody (14893; Cell signaling technology) and 1:1200 dilution of anti-human GAPDH antibody (2118; Cell signaling technology) with anti-rabbit secondary detection module (DM-001; Bio-Techne). Post run, data were analysed using “Compass for SW” software for chemiluminescent detection. The peak area for each band was used for the calculation of MLLT1 degradation with respect to DMSO control. Data is shown as “MLLT1% degradation”.
Category A: A≥75% degradation
Category B: 50%≤B<75% degradation
Category C: 25%≤C<50% degradation
Category D: D<25% degradation

TABLE 1

| | MLLT1% | MLLT1% | MLLT1% |
|---|---|---|---|
| Example | Degradation | Degradation | Degradation |
| Number | @ 1 μM | @ 0.1 μM | @ 0.01 μM |
| 1 | A | A | A |
| 2 | A | A | B |
| 3 | A | A | B |
| 4 | B | D | D |
| 5 | C | B | B |
| 6 | A | B | C |
| 9 | A | C | D |
| 10 | C | C | C |
| 11 | B | B | B |
| 15 | A | A | D |
| 16 | A | A | A |
| 18 | A | A | A |
| 20 | A | C | C |
| 21 | A | A | A |
| 22 | A | A | A |
| 23 | A | A | A |
| 24 | A | B | ND |
| 25 | A | A | ND |
| 26 | A | A | ND |
| 27 | A | A | A |
| 28 | C | C | ND |
| 29 | A | A | B |
| 30 | A | A | A |
| 31 | A | B | C |
| 32 | B | A | B |
| 33 | A | B | B |
| 34 | A | B | D |
| 35 | B | D | C |
| 36 | A | A | A |
| 38 | B | A | B |
| 39 | A | A | A |
| 40 | A | A | A |
| 41 | B | C | D |
| 42 | A | A | B |
| 43 | A | B | C |
| 44 | A | A | B |
| 45 | A | A | A |
| 46 | B | A | A |
| 47 | B | D | D |
| 48 | A | B | C |
| 49 | A | A | A |
| 50 | A | A | A |
| 51 | A | B | C |
| 52 | A | A | A |
| 53 | A | A | A |
| 54 | A | A | A |
| 55 | A | A | B |
| 56 | A | A | A |
| 57 | A | A | A |
| 58 | A | A | A |
| 59 | A | A | A |
| 60 | A | B | C |
| 61 | A | A | C |
| 62 | A | B | B |
| 63 | A | B | D |
| 65 | A | B | C |
| 66 | A | A | A |
| 67 | A | A | A |
| 68 | B | A | A |
| 69 | A | A | A |
| 70 | A | A | A |
| 71 | A | A | B |
| 72 | B | A | A |
| 73 | A | A | B |
| 74 | A | B | B |
| 98 | A | A | A |
| 99 | A | A | A |
| 102 | A | A | A |
| 103 | A | A | A |
| 104 | C | A | B |
| 105 | A | A | B |
| 106 | A | A | A |
| 107 | A | A | A |
| 108 | A | A | B |
| 109 | A | A | A |

ND = not determined

### WV4-11 Cellular Proliferative Assays

### Assay Format A

An MV4-11 proliferation assay was used to test the ability of compounds to reduce cell viability. MV4-11 cells (ATCC #CRL9591) were grown in IMDM medium (11510596, Gibco) supplemented with 10% fetal bovine serum (F7524, Gibco) and 1% Penicillin/Streptomycin (15140-122, Gibco) at 37° C. with 5% CO₂. Cells were seeded in 384-well Elplasia plates (4447, Corning) at a density of 50 cells/well and treated with vehicle (DMSO) or compounds (10 serial semi-log dilutions, 30 μM as top concentration).

After 120 hours of treatment, cells were incubated for 10 min at RT with the CellTiter-Glo (CTG) reagent (G7571, Promega). Measurement of the luminescence signal was performed on a microplate reader (Ensight, PerkinElmer). CTG reagent measures the amount of ATP, which is directly proportional to the number of cells, to determine the number of viable cells in culture.

The data were analysed as follows:

$$Data normalization , N ( x ) = \frac{x - 〈 cr 〉}{〈 sr 〉 - 〈 cr 〉} \times 100$$

where x is the measured raw signal value of a well, (cr) is the median of the measured signal values for the Central Reference (DMSO) wells on a plate, (sr) is the median of the measured signal values for the Scale Reference (reference compound) wells on a plate.

$$Curve fitting , {S}_{0} + \frac{{S}_{inf} - {S}_{0}}{1 + {( \frac{A {C}_{50}}{{10}^{c}} )}^{n}}$$

where the experimental set-up assumes the Hill parameters in certain ranges: S₀~Central Reference (CR) from normalization (=Bottom), S_{inf}~Scale Reference (SR) from normalization (=Top), nHill ~1, AC₅₀~within the measured concentration range.

### Assay Format B

MV4-11 cells at a confluency of ~50-80% was used for cell growth assay. 600 cells/well in a 384-well opaque cell culture plate (6007680; Perkin Elmer) were seeded in 40 μL of complete medium (IMDM+10% FBS+1× Penicillin-Streptomycin). Cells were incubated overnight at 37° C., 5% CO₂. 10 mM DMSO stocks were prepared, cells were treated with 10 concentrations of test compounds starting from 1 uM or 100 nM, 3-fold dilution in 10 uL (5× concentration) of complete medium. Final DMSO concentration was 0.3%. Culture plates were centrifuged at 40×g, 30s. Cells were incubated with compounds for 168 h (7 days). After 7 days, 20 uL CTG 2.0 reagent (G9242; Promega) was added per well and luminescence reading was taken in Envision Xcite 2105 multimode plate reader. Data was analyzed in GraphPad Prism 10.0 and data was shown as % viability with respect to DMSO control and dose-response curves were generated using four parameter logistic regression. Assay quality was determined by z-factor calculation using positive and negative controls.

IC₅₀ was <100 nM for each of the Examples set out in the Table below:

TABLE 2

| Example Number | Assay format |
|---|---|
| 1 | A |
| 2 | A |
| 3 | A |
| 7 | A |
| 8 | A |
| 11 | A |
| 12 | A |
| 13 | A |
| 14 | A |
| 15 | A |
| 16 | A |
| 17 | A |
| 18 | A |
| 19 | A |
| 21 | A |
| 22 | A |
| 23 | A |
| 25 | A |
| 26 | A |
| 27 | A |
| 29 | A |
| 30 | A |
| 31 | A |
| 32 | A |
| 33 | A |
| 36 | A |
| 38 | A |
| 39 | B |
| 40 | B |
| 42 | A |
| 44 | A |
| 45 | A |
| 46 | A |
| 47 | A |
| 48 | A |
| 52 | B |
| 53 | B |
| 54 | B |
| 55 | B |
| 56 | B |
| 57 | B |
| 58 | B |
| 59 | B |
| 61 | B |
| 66 | B |
| 67 | B |
| 68 | B |
| 69 | B |
| 70 | B |
| 71 | B |
| 72 | B |
| 74 | B |
| 98 | B |
| 99 | B |
| 100 | B |
| 101 | B |
| 102 | B |
| 103 | B |
| 104 | B |
| 105 | B |
| 106 | B |
| 107 | B |
| 108 | B |
| 109 | B |

### Comparison of PROTAC and Small Molecule Activity

Comparative Example compound 76 was prepared as described above and tested following the protocol of Assay Format A. The result is provided in Table 3 together with the results of a number of representative compounds of the invention.

TABLE 3

| Experimental | MV4-11 | Assay |
|---|---|---|
| Example No | antiproliferative IC₅₀ (nM) | method |
| 1 | 4 | A |
| 15 | 12 | A |
| 16 | 9 | A |
| 21 | 11 | A |
| 22 | 8 | A |
| 25 | 11 | A |
| 26 | 14 | A |
| 30 | 6 | A |
| 32 | 15 | A |
| 44 | 5 | A |
| 45 | 10 | A |
| 46 | 7 | A |
| 76 | 40 | A |

### MLLT1 and MLLT3 Inhibition Data

This procedure is intended to measure the interaction between YEATS proteins and histone peptides modified by an appropriate epigenetic mark and can be used to assay compounds which bind to the YEATS proteins and inhibit this interaction. The assay uses 6His tagged MLLT1 or MLLT3 with an anti-6His antibody conjugated with Eu³⁺ chelate (donor) and a biotinylated peptide with Streptavidin conjugated XL-665 (acceptor). When the donor and acceptor labels are in close proximity, by binding of the peptide by MLLT1 or MLLT3, the excitation of the donor (337 nm) triggers a Fluorescence Resonance Energy Transfer (FRET) to the acceptor which then fluoresces at specific wavelength (665 nm).

### HTRF Protein-Peptide Titration

This step optimises the protein/peptide ratio that will be used in HTRF screening assay in order to allow the best signal-to-noise ratio. Each new purified protein should be titrate this way as the signal-to-noise ratio may differ from different protein batches. Displacement assay for MLLT1 and MLLT3 was performed by using H3K9 cro peptide (ARTKQTAR(Kcrot)STGGKAPRKQLYK(biotin)).

A serial dilution of 4× solution of biotinylated peptide was prepared in assay buffer (25 mM HEPES, 20 mM NaCl, 0.05% BSA, 0.05% Tween-20, pH 7.0) covering concentrations from 3.2 μM to 0.2 nM. and 5 μL of each dilution were transferred horizontally to fill 15 columns of a white ProxiPlate-384 Plus shallow well Microplate (Perkin Elmer, USA). A sixteenth column of buffer was also included for a background reference. In parallel, a serial dilution of the 6His-tag protein of interest was prepared in the same way and 5 μL of each dilution were vertically added to the wells containing the peptide dilutions. Buffer has been transferred to the last line of the plate in order to make a no-protein reference. The microplate has been covered and incubated 1 hour at room temperature. A 2× detection mix was prepared in assay buffer by diluting streptavidin conjugated with XL-665 (610SAXLB, Cisbio Bioassays, France) to 1/2000 and anti-6His(Eu) chelate (LANCE Eu-W1024 Anti-6×His, ADO111, Perkinelmer LAS (UK)LTD) to 1/20000, 10 μL were added to each well and the plate has been incubated for 1 to 24 hours at room temperature in the dark. The plate was read in the Pherastar using the HTRF module with dual emission protocol (A=ex. 320 nm em. 665 nm, B=ex. 320 nm em. 620 nm).

The HTRF ratio was calculated as channel A/B*10000 and the specific signal as follow: specific signal=HTRF Ratio Positive signal—HTRF Ratio Negative signal (without protein). The concentrations of protein and peptide that allow the maximum specific signal without saturation were selected to perform HRTF screening assay described below.

### HTRF IC₅₀ Determination Assay

The compounds were dissolved in DMSO at a stock concentration of 10 mM and dispensed into microplate using the Echo acoustic liquid handler (Labcyte, Beckman Coulter, USA). The assay covers a window of compound concentrations from 20 μM to 20 nM spread in 11 data points in duplicate. Solvent and compound controls were added to each plate in order to calculate the background signal.

Ten microliters of 2× mix of 6His-protein and biotinylated peptide, the concentrations were determined above, were added to the dispensed compounds and incubated for one hour at room temperature in the dark. Ten microliters of the 2× detection mix was added to each well and the plate was incubated for another hour at room temperature in the dark and the plate was read in order to calculate the HTRF specific signal. The % inhibition values as follows:

$$\% I = \frac{positive control - signal}{positive control} \times 100$$

The IC₅₀ values were determined by plotting % inhibition vs compound concentration and fitting the data to a non-linear sigmoidal dose response equation (4 parameter fit):

$${I}_{i} = {I}_{o} + \frac{{I}_{complete} - {I}_{o}}{1 + {( \frac{{IC}_{50}}{{c}_{i}} )}^{S}}$$

Iᵢ: Inhibition at given concentration cᵢ
I_{complete}: Complete absence of binding, bottom of curve
I₀: No inhibition of binding, top of curve
IC₅₀: Concentration at which inhibition is 50%
cᵢ: concentration i in range tested
s: Hill coefficient (slope atIC₅₀)
   Calculations were carried out in Excel and GraphPad Prism templates.

### Results

Table 4 exhibits the ranges of IC₅₀ (nM) of the compounds when tested in the MLLT1 and MLLT3 assays.

Category A: IC₅₀≤100 nM; Category B: 100 nM<IC₅₀≤1000 nM; Category C: 1000 nM<IC₅₀≤10000 nM;

TABLE 4

| Example Number | MLLT1 HTRF IC₅₀ nM | MLLT3 HTRF IC₅₀ nM |
|---|---|---|
| 75 | A | B |
| 76 | A | A |
| 77 | B | C |
| 78 | C | C |
| 79 | A | A |
| 80 | A | A |
| 81 | B | C |
| 84 | B | B |
| 85 | C | ND |
| 86 | C | C |
| 87 | A | A |
| 88 | A | A |
| 89 | B | C |
| 90 | A | A |
| 91 | C | C |
| 92 | A | A |
| 93 | C | C |

Comparative Example compound 76 was prepared as described above and tested following the protocol of the HTRF IC₅₀ Assay. The result is provided in Table 5 together with the results of a number of representative compounds.

TABLE 5

| Example Number | MLLT1 HTRF IC₅₀ nM | MLLT3 HTRF IC₅₀ nM |
|---|---|---|
| 76 | 43 | 81 |
| 80 | 6 | 22 |
| 88 | 17 | 37 |
| 90 | 12 | 45 |
| 92 | 37 | 63 |

### Degradation of MLLT1 and MLLT3 by Example 1 in NIH/3T3 Cells

### MLLT1 Degradation Assay:

MLLT1 degradation efficacy of compounds was tested in an automated chemiluminescent detection system (JESS, Cat #004-650). NIH/3T3 (#CRL-1658, ATCC) were seeded at a density of 0.5 million cells/well in a cell culture treated 6-well cell culture plate in 2 mL of complete medium (DMEM+10% FBS+1× Penicillin-Streptomycin) and incubated at 37° C., 5% CO₂. The next day, 10 mM DMSO stocks were prepared, and cells were treated with eight concentrations of test compound, starting from 100 nM, 5-fold dilution, at a final DMSO concentration of 0.1% and incubated for 5 h at 37° C., 5% CO₂. Post incubation, cells were washed with HBSS and lysed in 70 μL of RIPA buffer supplemented with Protease inhibitors (sc-24948; Santa Cruz). Protein estimation was done using BCA protein assay kit (23228; Pierce). 4 gg protein lysate (4 μL of 1 μg/μL lysate) was loaded into each well of the JESS cartridge (SM-W004; Bio-techne) and the JESS run was performed using 1:800 dilution of anti-mouse MLLT1 antibody (H00004298-M01; Novus Bio) and 1:1200 dilution of anti-human GAPDH antibody (2118; Cell signalling technology) with anti-rabbit (DM-001; Bio-Techne) and anti-mouse secondary detection module (DM-002; Bio-Techne). Post run, data was analysed using “Compass for SW” software for chemiluminescent detection. Peak fit was done using the “dropped lines” method. Peak area for MLLT1 and GAPDH was used for the calculation of MLLT1 degradation with respect to DMSO control.”

### MLLT3 Degradation Assay:

MLLT3 degradation efficacy of compounds was tested in an automated chemiluminescent detection system (JESS, Cat #004-650). NIH/3T3 (#CRL-1658, ATCC) were seeded at a density of 0.5 million cells/well in a cell culture treated 6-well cell culture plate in 2 mL of complete medium (DMEM+10% FBS+1× Penicillin-Streptomycin) and incubated at 37° C., 5% CO₂. The next day, 10 mM DMSO stocks were prepared, and cells were treated with eight concentrations of test compounds, starting from 100 nM, 5-fold dilution, at a final DMSO concentration of 0.1% and incubated for 5 h at 37° C., 5% CO₂. Post incubation, cells were washed with HBSS and lysed in 70 μL of RIPA buffer supplemented with Protease inhibitors (sc-24948; Santa Cruz). Later, protein estimation was done using BCA protein assay kit (23228; Pierce). 4 gg protein lysate (4 μL of 1 μg/μL lysate) was loaded into each well of the JESS cartridge (SM-W004; Bio-techne) and the JESS run was performed using 1:200 dilution of anti-mouse MLLT3 antibody (#47577; CST) and 1:800 dilution of anti-human GAPDH antibody (2118; Cell signaling technology) with anti-rabbit secondary detection module (DM-001; Bio-Techne). Post run, data was analysed using “Compass for SW” software for chemiluminescent detection. Peak fit was done using “dropped lines” method. Peak area for MLLT3 and GAPDH was used for the calculation of MLLT3 degradation with respect to DMSO control. % Degradation for each protein was plotted against the respective doses (test compound concentration) and dose-response curve (DRC) was generated using four-parameter logistic equation (non-linear regression). The following parameters for each DRC was captured: DC₅₀ (concentration in nM at which degradation is 50%, interpolated from standard curve and transformed to linear scale), Dₘₐₓ (maximum % degradation observed). DC₅₀ and Dₘₐₓ are reported in Table 6 for Example 1.

TABLE 6

| | MLLT1 | MLLT1 | MLLT3 | MLLT3 |
|---|---|---|---|---|
| | DC₅₀ in | Dₘₐₓ in | DC₅₀ in | Dₘₐₓ in |
| | NIH/3T3 | NIH/3T3 | NIH/3T3 | NIH/3T3 |
| Example | cells (nM) | cells | cells (nM) | cells |
| 1 | 1.4 | 90% | 1.6 | 85% |

### MLLT1 DC₅₀ and Dₘₐₓ Determination and Antiproliferation Cell Activity for Examples 1 and 110 in MV4-11 Cells

### MLLT1 Degradation Assay:

MLLT1 degradation efficacy of compounds was tested in an automated chemiluminescent detection system (JESS, Cat #004-650). MV4-11 (#CRL-9591, ATCC) were seeded at a density of 1.2 million cells/well in a non-treated 6-well cell culture plate in 2.4 mL of complete medium (IMDM+10% FBS+1× Penicillin-Streptomycin). 10 mM DMSO stocks were prepared, and cells were treated with eight concentrations of test compounds, starting from 100 nM, 5-fold dilution, at a final DMSO concentration of 0.1% and incubated for 4 h at 37° C., 5% CO₂. Post incubation, cells were centrifuged, washed with HBSS and lysed in 60 μL of RIPA buffer supplemented with Protease inhibitors (sc-24948; Santa Cruz). Protein estimation was done using BCA protein assay kit (23228; Pierce). 4 gg protein lysate (4 μL of 1 μg/μL lysate) was loaded into each well of the JESS cartridge (SM-W004; Bio-techne) and the JESS run was performed using 1:900 dilution of anti-human MLLT1 antibody (ABE2596; Merck) and 1:1200 dilution of anti-human GAPDH antibody (2118; Cell signaling technology) with anti-rabbit secondary detection module (DM-001; Bio-Techne). Post run, data was analysed using “Compass for SW” software for chemiluminescent detection. Peak fit was done using “dropped lines” method. Peak area for MLLT1 and GAPDH was used for the calculation of MLLT1 degradation with respect to DMSO control. Data was shown as “% degradation”. % Degradation was plotted against the respective doses (test compound concentration) and dose-response curve (DRC) was generated using four-parameter logistic equation (non-linear regression). Following parameters for each DRC was captured: DC₅₀ (concentration in nM at which degradation is 50%, interpolated from standard curve and transformed to linear scale), Dₘₐₓ (maximum % degradation observed). DC₅₀ and Dₘₐₓ data are captured in Table 7 for Example 1 and Example 110, which is a comparative example.

TABLE 7

| | DC₅₀ in | Dₘₐₓ in | MV4-11 | MV4-11 |
|---|---|---|---|---|
| | MV4-11 | MV4-11 | Antiproliferation | Antiproliferation |
| Example | cells | cells | IC₅₀ (nM) | Assay Method |
| 1 | 0.5 | 87% | 6.5 | B |
| 110 | 41 | 58% | >1000 | B |

### Example 112: Efficacy In Vivo

### In Vivo Efficacy of Example 1 from an Oral Dose

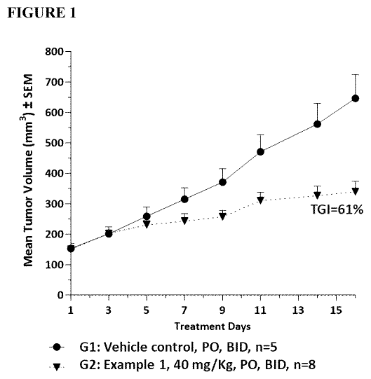

The MV4-11 subcutaneous model is an in vivo model of human AML (acute myeloid leukemia). Female NOD.SCID mice were injected subcutaneously (SC) in the right flank region with 0.2 mL (Serum free media: Matrigel; 1:1 ratio) of 5×10⁶ MV4-11 cell suspension. Tumor growth was measured by digital vernier caliper. Tumor size (volume) was measured on the day of randomization and thrice weekly until the end of the study. The tumor volume was determined by measurements of the longest axis (L) and the shortest axis (W) of tumor. Further, tumor volume was calculated by the formula: Tumor volume (mm³)=(Length×Width²)/2. Mice were randomized based on tumor volume 100-200 mm³ for efficacy evaluation. Group (G1): Vehicle Control (10 mL/kg), oral (PO), twice daily (BID); Group 2 (G2): Example 1, 40 mg/Kg, PO, BID for the indicated duration of the study. Tumor growth inhibition (TGI) calculated from tumor volume data using the following formula. TGI=(1−(Tt−T0)/(Ct−C0))×100% Tt and Ct were the mean tumor volumes of the treated and control animals on the day of treatment (measurement), and T0 and C0 were the mean tumor volumes of the treated and control animals at the start of the experiment. The TGI graph for Example 1 is shown in FIG. 1.

In one embodiment, the invention provides compounds according to the following aspects.

1. A compound which is a Proteolysis Targeting Chimera (PROTAC) or a pharmaceutically acceptable salt thereof, wherein the PROTAC has the structure:

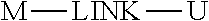

wherein U is an E3 ubiquitin ligase binding moiety, LINK is a moiety that covalently links M and U, and M is an MLLT1 and/or MLLT3 binder of formula (I):

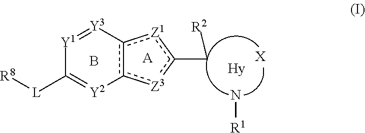

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
each R¹¹ is independently selected from H, C₁₋₄ alkyl, and C₁₋₄ cycloalkyl; wherein M is bonded to LINK via a C or N atom within group R⁸ such that a hydrogen atom on the C or N atom within group R⁸ is replaced with a bond to LINK.

2. A compound according to aspect 1, wherein LINK is (a) a single bond, or (b) a chemical linker group represented by formula (L):

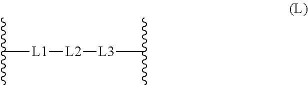

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynyl, —(C₂₋₆ alkenylene)-, a divalent 3- to 6-membered cycloalkyl ring and a divalent 4- to 7-membered heterocyclyl ring;
L2 is represented by the formula -(L4)ₘ-;
each L4 is independently selected from a divalent 3- to 6-membered cycloalkyl ring, a divalent 4- to 7-membered heterocyclyl ring and a unit of formula:

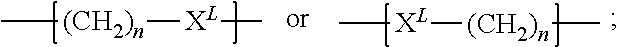

each X^{L} is independently selected from a single bond, —N(R′)—, —O—, —C(O)—, —S—, —SO—, —SO₂—, —C(R″)═C(R″)— and —C(H)—C(H)—;
n is selected from 1 to 4;
m is selected from 1 to 30;
each R′ is independently selected from H and C₁₋₄ alkyl; and
each R″ is independently selected from H and halo.

3. A compound according to aspect 2, wherein each L4 is independently selected from a divalent 4- to 7-membered heterocyclyl ring and a unit of formula

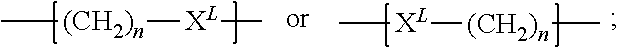

wherein each X^{L} is independently selected from a single bond, —O—, or —S—; and
n is selected from 1 or 2.

4. A compound according to aspect 2 or 3, wherein each divalent 4- to 7-membered heterocyclyl ring of L1, L2 and L3 when present contains at least one N atom.

5. A compound according to aspect 4, wherein each divalent 4- to 7-membered heterocyclyl ring of L1, L2 and L3 when present contains one or two N atoms.

6. A compound according to aspect 5, wherein each divalent 4- to 7-membered heterocyclyl ring of L1, L2 and L3 when present is independently selected from piperidinyl and diazinanyl.

7. A compound according to any one of aspects 2 to 6, wherein L2 is selected from —(CH₂)ₘ—, —(CH₂CH₂O)ₘ—, —(OCH₂CH₂)ₘ—, —(CH₂CH₂S)ₘ—, —(SCH₂CH₂)ₘ—, and a divalent 4- to 7-membered heterocyclyl ring.

8. A compound according to any one of aspects 2 to 7, wherein m is selected from 1 to 10.

9. A compound according to any one of aspects 2 to 8, wherein L1 and L3 are each independently selected from a single bond, —(C₁₋₆ alkylene)-, —N(R′)—, —C(O)N(R′)—, —O—, —N(R′)C(O)—, —C(O)—, —S(O₂)N(R′)—, —N(R′)S(O₂)—, ethynyl, and a divalent 4- to 7-membered heterocyclyl ring.

10. A compound according to any one of aspects 2 to 9, wherein L1 and L3 are each independently selected from a single bond, —(C₁₋₆ alkylene)-, —N(R′)—, —C(O)N(R′)—, —N(R′)C(O)—, —O—, ethynyl, and a divalent 4- to 7-membered heterocyclyl ring.

11. A compound according to any one of aspects 2 to 10, wherein L1 and L3 are each independently selected from a single bond, —(C₁₋₆ alkylene)-, —N(R′)—, —C(O)N(R′)—, —N(R′)C(O)—, —O—, ethynyl, piperidinyl and diazinanyl.

12. A compound according to any one of aspects 1 to 11, wherein U is a CRBN or VHL E3 ubiquitin ligase binding moiety.

13. A compound according to aspect 12, wherein U is a CRBN E3 ubiquitin ligase binding moiety.

14. A compound according to aspect 12, wherein U is a VHL E3 ubiquitin ligase binding moiety.

15. A compound according to any one of aspects 1 to 12, wherein U is:
(a) a CRBN E3 ubiquitin ligase binding moiety of formula (U1):

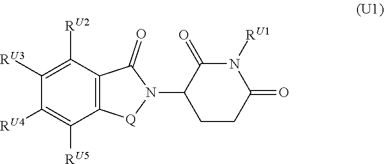

wherein:
R^{U1} is H;
Q is selected from —CH(R^{U6})—, —N(R^{U6})—, —O—, —C(O)—, —NH—CH(R^{U6})—,
N═C(R^{U6})—, or —N═N—;
R^{U6} is H or C₁₋₄alkyl;
R^{U2} and R^{U1} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

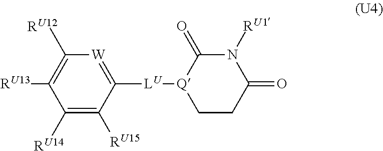

wherein:
R^{U1′} is H;
W is N or CR^{U16};
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U12}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

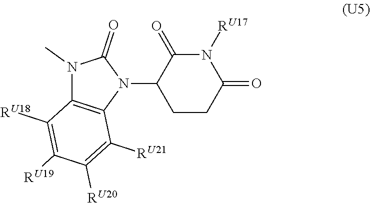

wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

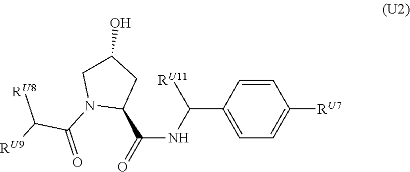

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

16. A compound according to aspect 15, wherein U is a VHL E3 ubiquitin ligase binding moiety of formula (U3):

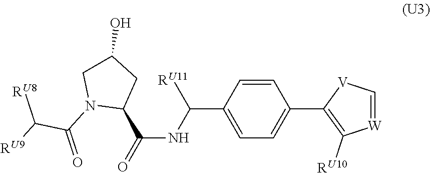

wherein:
V is S or O;
W is N or CH;
R^{U10} is H or C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

17. A compound according to aspect 16, wherein R^{U10} is H or methyl.

18. A compound according to any one of aspects 15 to 17, wherein R^{U8} is t-butyl.

19. A compound according to aspect 15, wherein U is a CRBN ubiquitin ligase binding moiety of formula (U1), wherein R^{U6} is H or methyl.

20. A compound according to aspect 15 or 19, wherein U is a CRBN ubiquitin ligase binding moiety of formula (U1), wherein Q is selected from —C(O)— and —CH₂—.

21. A compound according to any one of aspects 15, 19 and 20, wherein U is a CRBN ubiquitin ligase binding moiety of formula (U1), wherein three of R^{U2}, R^{U3}, R^{U4} and R^{U5} are H.

22. A compound according to aspect 15, wherein U is a CRBN ubiquitin ligase binding moiety of formula (U4), wherein Q′ is N.

23. A compound according to aspect 15 or 22, wherein U is a CRBN ubiquitin ligase binding moiety of formula (U4), wherein Q′ is CH.

24. A compound according to aspect 23, wherein L^{U} is a single bond.

25. A compound according to aspect 23, wherein L^{U} is —C(O)N(H)—, wherein the C atom of L^{U} is bonded to phenyl, and the N atom of L^{U} is bonded to Q′.

26. A compound according to any one of aspects 15 and 22 to 25, wherein U is a CRBN ubiquitin ligase binding moiety of formula (U4), wherein R^{U15} and R^{U16} are H, and two of R^{U12}, R^{U13} and R^{U14} are H.

27. A compound according to any one of aspects 15 and 22 to 25, wherein U is a CRBN ubiquitin ligase binding moiety of formula (U4), wherein R^{U13}, R^{U14} and R^{U15} are H, R^{U12} is a single bond to LINK, and R^{U16} is selected from H, F and Me.

28. A compound according to aspect 15, wherein U is a CRBN ubiquitin ligase binding moiety of formula (U5), wherein three of R^{U18}, R^{U19}, R^{U20} and R^{U21} are H.

29. A compound according to any one of the preceding aspects, wherein the N atom of ring Hy which is substituted by R¹ is adjacent to the C atom of ring Hy that is bonded to ring A.

30. A compound according to any one of the preceding aspects, wherein no more than two of Y¹, Y² and Y³ are N.

31. A compound according to any one of the preceding aspects, wherein Z¹ is —C(R⁴)— and Z³ is —N(H)—.

32. A compound according to any one of the preceding aspects, wherein Z¹ is —C(R⁴)—, Z³ is —N(H)—, Y¹ is N, Y² is —C(R⁶)— and Y³ is —C(R⁵)—.

33. A compound according to any one of the preceding aspects, wherein X is a bond, —N(Me)-, O or —CH₂—.

34. A compound according to any one of the preceding aspects, wherein X is a bond.

35. A compound according to any one of the preceding aspects, wherein M is of formula (II):

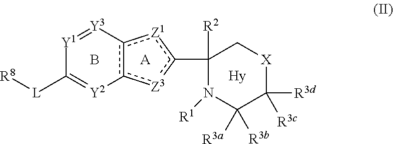

wherein:
Z¹, Z³, Y¹, Y², Y³, R¹, R², R⁸, X and L are as defined in any one of the preceding aspects;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)-then neither R^{3c} nor R^{3d} are halo;
wherein at least two of R^{3a}, R^{3b}, R^{3c} and R^{3d} are H; and
wherein M is bonded to LINK via a C or N atom within group R⁸ such that a hydrogen atom on
the C or N atom within group R⁸ is replaced with a bond to LINK.

36. A compound according to any one of the preceding aspects, wherein R⁴, R⁵ and R⁶ are H.

37. A compound according to any one of aspects 1 to 35, wherein one of R⁴, R⁵ and R⁶ is not H.

38. A compound according to aspect 37, wherein one of R⁴, R⁵ and R⁶ is selected from CN, fluoro, chloro, methoxy, methyl and trifluoromethyl, and the rest are H, with the proviso that R⁵ and R⁶ are not CN.

39. A compound according to any one of aspects 1 to 28, wherein M is of formula (III):

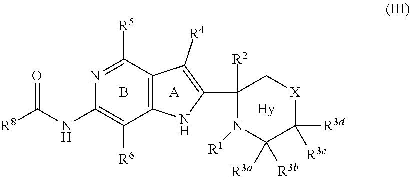

wherein:
R¹ is H or C₁₋₄ alkyl which is itself unsubstituted or substituted with one C₁₋₄ alkoxy or one, two or three halo, preferably with one C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3a} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring,
with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo, C₁₋₄ alkoxy, and C₁₋₄ alkyl which is itself unsubstituted or substituted by one, two or three halo;
R⁵ and R⁶ are independently selected from H, halo, C₁₋₄ alkoxy, and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
wherein M is bonded to LINK via a C or N atom within group R⁸ such that a hydrogen atom on the C or N atom within group R⁸ is replaced with a bond to LINK.

40. A compound according to aspect 39, wherein the C or N atom within group R⁸ that is bonded to LINK is a C or N atom of the heteroaryl ring of group R⁸, or a C or N atom of the heteroaryl, cycloalkyl, heterocyclyl or phenyl ring of R¹⁰.

41. A compound according to any one of aspects 1 to 40, wherein R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl and cyclopropyl, most preferably cyclopropyl.

42. A compound according to aspect 41, wherein R⁸ is an 8- to 10-membered heteroaryl ring which is unsubstituted or substituted by one or two C₁₋₄ alkyl.

43. A compound according to any one of the preceding aspects, wherein Hy is an unsubstituted pyrrolidine ring, R¹ is methyl, and R² is H.

44. A compound according to aspect 1, wherein the PROTAC has a structure selected from:
1-(9-(2-(2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
rac-1-(8-{[2-(2,6-dioxopiperidin-3-yl)-1,3-dioxoisoindol-4-yl]amino}octyl)-3-methyl-N-[2-(1-methylpyrrolidin-2-yl)-1H-pyrrolo[3,2-c]pyridin-6-yl]indazole-6-carboxamide;
1-(8-{[2-(2,6-dioxopiperidin-3-yl)-1,3-dioxoisoindol-4-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-4-yl]amino}octyl)-3-methyl-N-{2-[(2S)-1-methylpyrrolidin-2-yl]-1H-pyrrolo[3,2-c]pyridin-6-yl}indazole-6-carboxamide;
2-(8-((2-(2,6-dioxopiperidin-3-yl)-1,3-dioxoisoindolin-4-yl)amino)octyl)-3-methyl-N-(2-((R)-1-methylpyrrolidin-2-yl)-1H-pyrrolo[3,2-c]pyridin-6-yl)-2H-indazole-6-carboxamide;
3-[4-({4-[2-(2,6-dioxopiperidin-3-yl)-6-fluoro-1,3-dioxoisoindol-5-yl]piperazin-1-yl}methyl)piperidin-1-yl]-1-methyl-N-{2-[(2R)-1-methylpyrrolidin-2-yl]-1H-pyrrolo[3,2-c]pyridin-6-yl}indazole-5-carboxamide;
1-[1-(3-{[2-(2,6-dioxopiperidin-3-yl)-1,3-dioxoisoindol-4-yl]amino}propyl)piperidin-4-yl]-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-5-yl]oxy}octyl)-3-methyl-N-{2-[(2R)-1-methylpyrrolidin-2-yl]-1H-pyrrolo[3,2-c]pyridin-6-yl}indazole-6-carboxamide;
1-{8-[3-(2,4-dioxo-1,3-diazinan-1-yl)-2-methylphenoxy]octyl}-3-methyl-N-{2-[(2R)-1-methylpyrrolidin-2-yl]-1H-pyrrolo[3,2-c]pyridin-6-yl}indazole-6-carboxamide;
1-{6-[3-(2,4-dioxo-1,3-diazinan-1-yl)-2-methylphenoxy]hexyl}-3-methyl-N-{2-[(2R)-1-methylpyrrolidin-2-yl]-1H-pyrrolo[3,2-c]pyridin-6-yl}indazole-6-carboxamide;
1-{7-[1-(2,6-dioxopiperidin-3-yl)-3-methyl-2-oxo-1,3-benzodiazol-4-yl]hept-6-yn-1-yl}-3-methyl-N-{2-[(2R)-1-methylpyrrolidin-2-yl]-1H-pyrrolo[3,2-c]pyridin-6-yl}indazole-6-carboxamide;
1-(8-((2-(2,6-dioxopiperidin-3-yl)-1,3-dioxoisoindolin-4-yl)oxy)octyl)-3-methyl-N-(2-((R)-1-methylpyrrolidin-2-yl)-1H-pyrrolo[3,2-c]pyridin-6-yl)-1H-indazole-6-carboxamide;
1-(3-((2-(2,6-dioxopiperidin-3-yl)-1,3-dioxoisoindolin-4-yl)amino)propyl)-3-methyl-N-(2-((R)-1-methylpyrrolidin-2-yl)-1H-pyrrolo[3,2-c]pyridin-6-yl)-1H-indazole-6-carboxamide;
1-(8-{[2-(2,6-dioxopiperidin-3-yl)-1,3-dioxoisoindol-5-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-5-yl]amino}octyl)-3-methyl-N-{2-[(2S)-1-methylpyrrolidin-2-yl]-1H-pyrrolo[3,2-c]pyridin-6-yl}indazole-6-carboxamide;
1-(8-{[2-(2,6-dioxopiperidin-3-yl)-1-oxo-3H-isoindol-4-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1-oxo-3H-isoindol-4-yl]amino}octyl)-3-methyl-N-{2-[(2S)-1-methylpyrrolidin-2-yl]-1H-pyrrolo[3,2-c]pyridin-6-yl}indazole-6-carboxamide;
N-(3-chloro-2-((R)-1-methylpyrrolidin-2-yl)-1H-pyrrolo[3,2-c]pyridin-6-yl)-1-(8-((2-(2,6-dioxopiperidin-3-yl)-1,3-dioxoisoindolin-4-yl)amino)octyl)-3-methyl-1H-indazole-6-carboxamide;
1-(4-{4-[2-(2,6-dioxopiperidin-3-yl)-1,3-dioxoisoindol-5-yl]piperazin-1-yl}butyl)-3-methyl-N-{2-[(2R)-1-methylpyrrolidin-2-yl]-1H-pyrrolo[3,2-c]pyridin-6-yl}indazole-6-carboxamide;
1-(4-{4-[2-(2,6-dioxopiperidin-3-yl)-1,3-dioxoisoindol-5-yl]piperazin-1-yl}butyl)-3-methyl-N-{2-[(2S)-1-methylpyrrolidin-2-yl]-1H-pyrrolo[3,2-c]pyridin-6-yl}indazole-6-carboxamide;
1-(4-((2-(2,6-dioxopiperidin-3-yl)-1,3-dioxoisoindolin-4-yl)amino)butyl)-3-methyl-N-(2-((R)-1-methylpyrrolidin-2-yl)-1H-pyrrolo[3,2-c]pyridin-6-yl)-1H-indazole-6-carboxamide;
1-(6-{[2-(2,6-dioxopiperidin-3-yl)-1,3-dioxoisoindol-4-yl]amino}hexyl)-3-methyl-N-{2-[(2R)-1-methylpyrrolidin-2-yl]-1H-pyrrolo[3,2-c]pyridin-6-yl}indazole-6-carboxamide;
1-(5-(4-(2-(2,6-dioxopiperidin-3-yl)-6-fluoro-1,3-dioxoisoindolin-5-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide;
1-(2-(2-(4-(2-(2,6-dioxopiperidin-3-yl)-6-fluoro-1,3-dioxoisoindolin-5-yl)piperazin-1-yl)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(2-(2-(2-((2-(2,6-dioxopiperidin-3-yl)-1,3-dioxoisoindolin-4-yl)amino)ethoxy)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(2-(2-((2-(2,6-dioxopiperidin-3-yl)-1,3-dioxoisoindolin-4yl)amino)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(3-(1-(2-(2,6-dioxopiperidin-3-yl)-1,3-dioxoisoindolin-4-yl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide;
1-(3-(1-(4-((2,6-dioxopiperidin-3-yl)carbamoyl)-2-fluorophenyl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide;
1-(5-(1-(2-(2,6-dioxopiperidin-3-yl)-1,3-dioxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide;
1-(7-(2-(2,6-dioxopiperidin-3-yl)-1-oxoisoindolin-4-yl) hept-6-yn-1-yl)-3-methyl-N-(2-((R)-1-methylpyrrolidin-2-yl)-1H-pyrrolo [3,2-c]pyridin-6-yl)-1H-indazole-6-carboxamide;
1-(3-((3-(2-(2,6-dioxopiperidin-3-yl)-1-oxoisoindolin-4-yl)prop-2-yn-1-yl)oxy)propyl)-3-methyl-N-(2-((R)-1-methylpyrrolidin-2-yl)-1H-pyrrolo[3,2-c]pyridin-6-yl)-1H-indazole-6-carboxamide;
1-(5-(2-(2,6-dioxopiperidin-3-yl)-1-oxoisoindolin-4-yl)pent-4-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(2-(2,6-dioxopiperidin-3-yl)-1,3-dioxoisoindolin-4-yl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(3-((2,6-dioxopiperidin-3-yl)carbamoyl)-2-fluorophenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(3-((2,6-dioxopiperidin-3-yl)carbamoyl)-2-methylphenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(8-((2-(2,6-dioxopiperidin-3-yl)-1,3-dioxoisoindolin-4-yl)amino)octyl)-N-(2-((R)-1-methylpyrrolidin-2-yl)-1H-pyrrolo[3,2-c]pyridin-6-yl)-1H-indazole-5-carboxamide;
1-(8-((2-(2,6-dioxopiperidin-3-yl)-1,3-dioxoisoindolin-4-yl)amino)octyl)-N-(2-((S)-1-methylpyrrolidin-2-yl)-1H-pyrrolo[3,2-c]pyridin-6-yl)-1H-indazole-5-carboxamide;
1-(9-(2-(2,6-dioxopiperidin-3-yl)-1-oxoisoindolin-4-yl)non-8-yn-1-yl)-N-(2-((R)-1-methylpyrrolidin-2-yl)-1H-pyrrolo[3,2-c]pyridin-6-yl)-1H-indazole-5-carboxamide;
1-(9-(1-(2,6-dioxopiperidin-3-yl)-3-methyl-2-oxo-2,3-dihydro-1H-benzo[d]imidazol-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-((E)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-((Z)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-(9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-(9-(6-(((S)-2,6-dioxopiperidin-3-yl)carbamoyl)pyridin-3-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(6-(((S)-2,6-dioxopiperidin-3-yl)carbamoyl)pyridin-2-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(((S)-2,6-dioxopiperidin-3-yl)carbamoyl)pyridin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(2-((R)-1-(methyl-d3)pyrrolidin-2-yl)-1H-pyrrolo[3,2-c]pyridin-6-yl)-1H-indazole-6-carboxamide;
1-(8-(2-(2,6-dioxopiperidin-3-yl)-1-oxoisoindolin-4-yl)oct-7-yn-1-yl)-3-methyl-N-(2-((R)-1-methylpyrrolidin-2-yl)-1H-pyrrolo[3,2-c]pyridin-6-yl)-1H-indazole-6-carboxamide;
1-(6-(2-(2,6-dioxopiperidin-3-yl)-1-oxoisoindolin-4-yl)hex-5-yn-1-yl)-3-methyl-N-(2-((R)-1-methylpyrrolidin-2-yl)-1H-pyrrolo[3,2-c]pyridin-6-yl)-1H-indazole-6-carboxamide;
1-(5-(4-(2-(2,6-dioxopiperidin-3-yl)-1,3-dioxoisoindolin-4-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-N-(2-((R)-1-ethylpyrrolidin-2-yl)-1H-pyrrolo[3,2-c]pyridin-6-yl)-3-methyl-1H-indazole-6-carboxamide;
1-(5-((3-(2-(2,6-dioxopiperidin-3-yl)-1-oxoisoindolin-4-yl)prop-2-yn-1-yl)oxy)pentyl)-3-methyl-N-(2-((R)-1-methylpyrrolidin-2-yl)-1H-pyrrolo[3,2-c]pyridin-6-yl)-1H-indazole-6-carboxamide;
1-(2-((6-(2-(2,6-dioxopiperidin-3-yl)-1-oxoisoindolin-4-yl)hex-5-yn-1-yl)oxy)ethyl)-3-methyl-N-(2-((R)-1-methylpyrrolidin-2-yl)-1H-pyrrolo[3,2-c]pyridin-6-yl)-1H-indazole-6-carboxamide;
1-(9-(3-(((S)-2,6-dioxopiperidin-3-yl)carbamoyl)-2-fluorophenyl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-7-fluoro-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
N-(3-chloro-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide;
1-(10-(2-(2,6-dioxopiperidin-3-yl)-1-oxoisoindolin-4-yl) dec-9-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrol[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(2-((R)-5-methyl-5-azaspiro[2.4]heptan-6-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(3-methyl-2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
3-cyclopropyl-1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrolo [3,2-c]pyridin-6-yl)-1H-indazole-6-carboxamide;
1-(9-(2-(2,6-dioxopiperidin-3-yl)-6-fluoro-1-oxoisoindolin-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(3-(2,6-dioxopiperidin-3-yl)-2-fluorophenyl) non-8-yn-1-yl)-3-methyl-N-(2-((R)-1-methylpyrrolidin-2-yl)-1H-pyrrolo [3,2-c]pyridin-6-yl)-1H-indazole-6-carboxamide;
1-(9-(6-(2,6-dioxopiperidin-3-yl) pyridin-2-yl) non-8-yn-1-yl)-3-methyl-N-(2-((R)-1-methylpyrrolidin-2-yl)-1H-pyrrolo [3,2-c]pyridin-6-yl)-1H-indazole-6-carboxamide;
(R)-1-(9-(3-(2,4-dioxotetrahydropyrimidin-1 (2H)-yl)-2-fluorophenyl) non-8-yn-1-yl)-3-methyl-N-(2-(1-methylpyrrolidin-2-yl)-1H-pyrrolo [3,2-c]pyridin-6-yl)-1H-indazole-6-carboxamide;
(R)-1-(9-(6-(2,4-dioxotetrahydropyrimidin-1 (2H)-yl)pyridin-2-yl)non-8-yn-1-yl)-3-methyl-N-(2-(1-methylpyrrolidin-2-yl)-1H-pyrrolo[3,2-c]pyridin-6-yl)-1H-indazole-6-carboxamide;
(R)-1-(9-(3-(2,4-dioxotetrahydropyrimidin-1 (2H)-yl)-5-fluoro-1-methyl-1H-indazol-6-yl)non-8-yn-1-yl)-3-methyl-N-(2-(1-methylpyrrolidin-2-yl)-1H-pyrrolo[3,2-c]pyridin-6-yl)-1H-indazole-6-carboxamide;
N-(3-cyano-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide;
N-(3-chloro-2-((R)-1-methylpyrrolidin-2-yl)-1H-pyrrolo[3,2-c]pyridin-6-yl)-1-(9-(2-(2,6-dioxopiperidin-3-yl)-7-fluoro-1-oxoisoindolin-4-yl)non-8-yn-1-yl)-3-methyl-1H-indazole-6-carboxamide;
1-(1-(3-(2-(2,6-dioxopiperidin-3-yl)-1-oxoisoindolin-4-yl)prop-2-yn-1-yl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-6-fluoro-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-1,3-dioxoisoindolin-4-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(3-(2-(2,6-dioxopiperidin-3-yl)-1-oxoisoindolin-4-yl)prop-2-yn-1-yl)-3-methyl-N-(2-((R)-1-methylpyrrolidin-2-yl)-1H-pyrrolo[3,2-c]pyridin-6-yl)-1H-indazole-6-carboxamide; 1-(6-(1-(6-(((S)-2,6-dioxopiperidin-3-yl)carbamoyl)pyridin-2-yl)piperidin-4-yl)hexyl)-3-methyl-N-(2-((R)-1-methylpyrrolidin-2-yl)-1H-pyrrolo[3,2-c]pyridin-6-yl)-1H-indazole-6-carboxamide; and
1-(4-(5-(2-(2,6-dioxopiperidin-3-yl)-1-oxoisoindolin-4-yl) pent-4-yn-1-yl) cyclohexyl)-3-methyl-N-(2-((R)-1-methylpyrrolidin-2-yl)-1H-pyrrolo[3,2-c]pyridin-6-yl)-1H-indazole-6-carboxamide.

45. A compound according to any one of aspects 1 to 28, wherein M is of formula (IV):

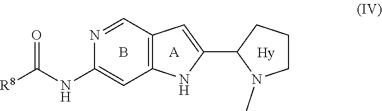

wherein R⁸ is an 8- to 10-membered heteroaryl ring, the group R⁸ being unsubstituted or substituted by one, two or three substituents independently selected from halo, C₁₋₄ alkyl and R¹⁰;
R¹⁰ is a 3-membered cycloalkyl ring;
wherein M is bonded to LINK via a C or N atom within group R⁸ such that a hydrogen atom on the C or N atom within group R⁸ is replaced with a bond to LINK.

46. A compound according to any one of aspects 1 to 45, wherein R⁸ is indazolyl.

47. A compound according to any one of the preceding aspects, wherein the group R⁸ is unsubstituted or substituted by one cyclopropyl group or one or two methyl groups.

48. A compound according to any one of the preceding aspects, wherein the group R⁸ is an indazolyl group which is unsubstituted or substituted by one or two methyl groups.

49. A compound according to any one of aspects 48 to 50, wherein R⁸ is selected from one of the following structures:

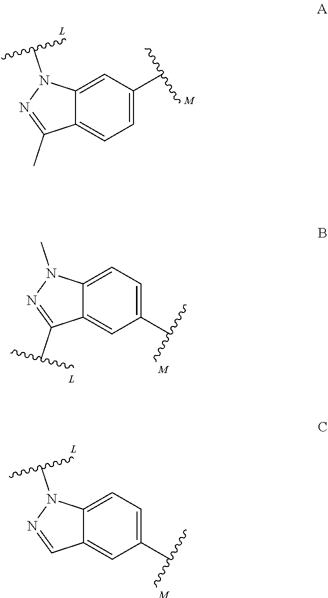

wherein:

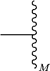

represents the point of attachment of R⁸ to the rest of moiety M;

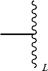

represents the point of attachment of R⁸ to LINK.

50. A compound according to any one of aspects 11 to 35 and 39 to 43, wherein at least one of R⁴, R⁵ and R⁶ is not H.

51. A compound which is a bicyclic compound of formula (I′) or a pharmaceutically acceptable salt thereof:

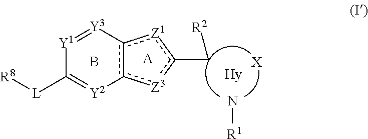

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)-, O, S, —S(O)₂—, —S(O)(NR¹¹)—, or —C(R¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R is selected from H, halo, C₁₋₄ alkoxy, C₃₋₅ cycloalkyl and C₁₋₄ alkyl which is itself unsubstituted or substituted by one, two or three halo;
at least one of R⁴, R⁵ and R is not H;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
each R¹¹ is independently selected from H, C₁₋₄ alkyl, and C₁₋₄ cycloalkyl.

52. A compound according to any one of the preceding aspects, wherein the bond from ring Hy to ring A is in the “up” position and the bond from ring Hy to R² is in the “down” position relative to ring Hy, as depicted below:

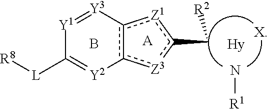

53. A compound according to aspect 51 which is selected from:
N-{3-chloro-2-[(2R)-1-methylpyrrolidin-2-yl]-1H-pyrrolo[3,2-c]pyridin-6-yl}-1,3-dimethylindazole-6-carboxamide;
N-{3-chloro-2-[(2S)-1-methylpyrrolidin-2-yl]-1H-pyrrolo[3,2-c]pyridin-6-yl}-1,3-dimethylindazole-6-carboxamide;
(R)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide;
(S)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide;
N-{3-fluoro-2-[(rel-2S)-1-methylpyrrolidin-2-yl]-1H-pyrrolo[3,2-c]pyridin-6-yl}-1-methylindazole-5-carboxamide;
N-{3-fluoro-2-[(rel-2R)-1-methylpyrrolidin-2-yl]-1H-pyrrolo[3,2-c]pyridin-6-yl}-1-methylindazole-5-carboxamide;
N-{3-fluoro-2-[(rel-2R)-1-methylpyrrolidin-2-yl]-1H-pyrrolo[3,2-c]pyridin-6-yl}-1,3-dimethylindazole-6-carboxamide;
N-{3-fluoro-2-[(rel-2S)-1-methylpyrrolidin-2-yl]-1H-pyrrolo[3,2-c]pyridin-6-yl}-1,3-dimethylindazole-6-carboxamide;
1,3-dimethyl-N-{3-methyl-2-[(rel-2R)-1-methylpyrrolidin-2-yl]-1H-pyrrolo[3,2-c]pyridin-6-yl}indazole-6-carboxamide;
1,3-dimethyl-N-{3-methyl-2-[(rel-2S)-1-methylpyrrolidin-2-yl]-1H-pyrrolo[3,2-c]pyridin-6-yl}indazole-6-carboxamide;
1,3-dimethyl-N-{4-methyl-2-[(rel-2R)-1-methylpyrrolidin-2-yl]-1H-pyrrolo[3,2-c]pyridin-6-yl}indazole-6-carboxamide;
1,3-dimethyl-N-{4-methyl-2-[(rel-2S)-1-methylpyrrolidin-2-yl]-1H-pyrrolo[3,2-c]pyridin-6-yl}indazole-6-carboxamide; and
(R)—N-(3-cyano-2-(1-methylpyrrolidin-2-yl)-1H-pyrrolo[3,2-c]pyridin-6-yl)-1,3-dimethyl-1H-indazole-6-carboxamide;
   and the pharmaceutically acceptable salts thereof.

54. A pharmaceutical composition which comprises a compound as defined in any one of aspects 1 to 53 and a pharmaceutically acceptable carrier or diluent.

55. A compound as defined in any one of aspects 1 to 53 for use in the treatment of the human or animal body by therapy.

56. A compound as defined in any one of aspects 1 to 53 for use in the treatment of cancer.

57. The compound for use according to aspect 56, wherein the cancer is a transcriptionally addicted cancer, such as breast cancer, acute leukemia, prostate cancer, bladder cancer, cholangiocarcinoma, colon adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, pheochromocytoma and paraganglioma, rectum adenocarcinoma, stomach adenocarcinoma, uterine corpus endometrial carcinoma, cervical squamous cell carcinoma and endocervical adenocarcinoma, lung squamous cell carcinoma, neuroblastoma, midline glioma or sarcoma, preferably neuroblastoma or midline glioma.

58. The compound for use according to aspect 56, wherein the cancer is acute myeloid leukaemia (AML), myelodysplastic syndrome (MDS), myeloproliferative neoplasm (MPN) or acute lymphoblastic leukaemia (ALL).

59. The compound for use according to aspect 58, wherein the cancer is mixed-lineage leukaemia (MLL) or rearranged acute leukemia (AML and ALL).

60. The compound for use according to aspect 58, wherein the cancer is NPM1 mutant acute leukemia.

101. A compound which is a Proteolysis Targeting Chimera (PROTAC) or a pharmaceutically acceptable salt thereof, wherein the PROTAC has the structure:

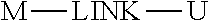

wherein U is an E3 ubiquitin ligase binding moiety, LINK is a moiety that covalently links M and U, and M is an MLLT1 and/or MLLT3 binder of formula (I):

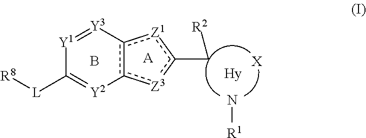

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
each R¹¹ is independently selected from H, C₁₋₄ alkyl, and C₁₋₄ cycloalkyl; wherein M is bonded to LINK via a C or N atom within group R⁸ such that a hydrogen atom on the C or N atom within group R⁸ is replaced with a bond to LINK.

102. A compound according to aspect 101, wherein LINK is (a) a single bond, or (b) a chemical linker group represented by formula (L):

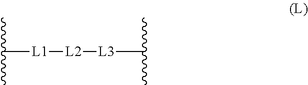

wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one or two C₁₋₄ alkyl;
L2 is represented by the formula -(L4)ₘ-;
each L4 is independently a unit of formula:

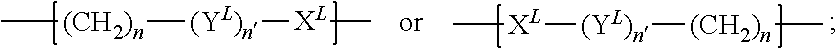

each X^{L} is independently selected from a single bond, —N(R′)—, —O—, —C(O)—, —S—, —SO—, —SO₂—, —C(R″)═C(R″)— and —C≡C—;
each Y^{L} is independently selected from a divalent 3- to 6-membered cycloalkyl ring, a divalent 4- to 7-membered heterocyclyl ring, or one of the following structures:

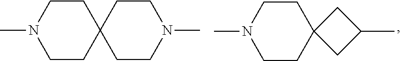

the cycloalkyl and heterocyclyl rings being unsubstituted or substituted by one or two C₁₋₄ alkyl; n is selected from 0 to 4;
n′ is selected from 0 or 1;
n+n′>1;
m is selected from 1 to 30;
each R′ is independently selected from H and C₁₋₄ alkyl; and
each R¹¹ is independently selected from H and halo.

103. A compound according to aspect 102, wherein each Y^{L} is a divalent 4- to 7-membered heterocyclyl ring, each X^{L} is independently selected from a single bond, —O—, or —S—, and n is selected from 1 or 2.

104. A compound according to aspect 102 or 103, wherein each divalent 4- to 7-membered heterocyclyl ring of L1, L2 and L3 when present contains at least one N atom.

105. A compound according to aspect 104, wherein each divalent 4- to 7-membered heterocyclyl ring of L1, L2 and L3 when present contains one or two N atoms.

106. A compound according to aspect 105, wherein each divalent 4- to 7-membered heterocyclyl ring of L1, L2 and L3 when present is independently selected from piperidinyl and diazinanyl.

107. A compound according to any one of aspects 102 to 106, wherein L2 is selected from —(CH₂)ₘ—, —(CH₂CH₂O)ₘ—, —(OCH₂CH₂)ₘ—, —(CH₂CH₂S)ₘ—, —(SCH₂CH₂)ₘ—, and a divalent 4- to 7-membered heterocyclyl ring.

108. A compound according to any one of aspects 102 to 107, wherein m is selected from 1 to 10.

109. A compound according to any one of aspects 102 to 108, wherein L1 and L3 are each independently selected from a single bond, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂0.6 alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one methyl group.

110. A compound according to any one of aspects 102 to 109, wherein L1 and L3 are each independently selected from a single bond, —(C₁₋₆ alkylene)-, —C(O)N(R′)—, —N(R′)C(O)—, —O—, ethynylene, and a divalent 4- to 7-membered heterocyclyl ring.

111. A compound according to any one of aspects 102 to 110, wherein L1 and L3 are each independently selected from a single bond, —(C₁₋₆ alkylene)-, —C(O)N(R′)—, —N(R′)C(O)—, —O—, ethynylene, piperidinyl and diazinanyl.

112. A compound according to any one of aspects 101 to 111, wherein U is a CRBN or VHL E3 ubiquitin ligase binding moiety.

113. A compound according to aspect 112, wherein U is a CRBN E3 ubiquitin ligase binding moiety.

114. A compound according to aspect 112, wherein U is a VHL E3 ubiquitin ligase binding moiety.

115. A compound according to any one of aspects 101 to 112, wherein U is:
(a) a CRBN E3 ubiquitin ligase binding moiety of formula (U1):

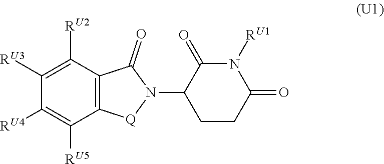

wherein:
R^{U1} is H;
Q is selected from —CH(R^{U6})—, —N(R^{U6})—, —O—, —C(O)—, —NH—CH(R^{U6})—
N═C(R^{U6})—, or —N═N—;
R^{U6} is H or C₁₋₄ alkyl;
R^{U2} and R^{U5} are each independently selected from H, halogen, C₁₋₄alkyl, NH₂, NO₂, OH, COOH, CN, CF₃, and a single bond to LINK;
R^{U3} and R^{U4} are each independently selected from H, halogen, C₁₋₄alkyl, NH₂, NO₂, OH, COOH, CN, CF₃, and a single bond to LINK;
wherein one and only one of R^{U2}, R^{U3}, R^{U4} and R^{U5} is a single bond to LINK;
(b) a CRBN E3 ubiquitin ligase binding moiety of formula (U4):

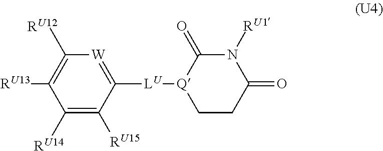

wherein:
R^{U1′} is H;
W is N or CR^{U16};
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U2}, R^{U13} and R^{U14} is a single bond to LINK; R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5):

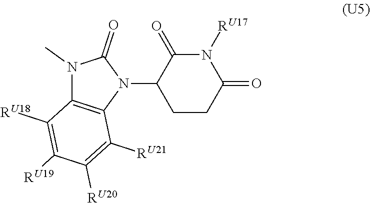

wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK;
or (d) a VHL E3 ubiquitin ligase binding moiety of formula (U2):

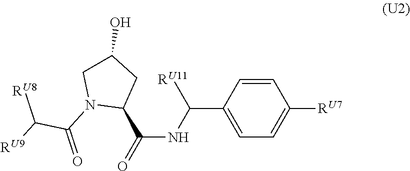

wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

116. A compound according to aspect 115, wherein U is a VHL E3 ubiquitin ligase binding moiety of formula (U3):

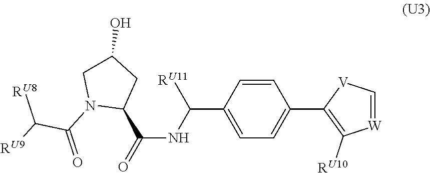

wherein:
V is S or O;
W is N or CH;
R^{U10} is H or C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

117. A compound according to aspect 116, wherein R^{U10} is H or methyl.

118. A compound according to any one of aspects 115 to 117, wherein R^{U8} is t-butyl.

119. A compound according to aspect 115, wherein U is a CRBN ubiquitin ligase binding moiety of formula (U1), wherein R^{U6} is H or methyl.

120. A compound according to aspect 115 or 119, wherein U is a CRBN ubiquitin ligase binding moiety of formula (U1), wherein Q is selected from —C(O)— and —CH₂—.

121. A compound according to any one of aspects 115, 119 and 120, wherein U is a CRBN ubiquitin ligase binding moiety of formula (U1), wherein three of R^{U2}, R^{U3}, R^{U4} and R^{U5} are H.

122. A compound according to aspect 115, wherein U is a CRBN ubiquitin ligase binding moiety of formula (U4), wherein Q′ is N.

123. A compound according to aspect 115 or 122, wherein U is a CRBN ubiquitin ligase binding moiety of formula (U4), wherein Q′ is CH.

124. A compound according to aspect 123, wherein L^{U} is a single bond.

125. A compound according to aspect 123, wherein L^{U} is —C(O)N(H)—, wherein the C atom of L^{U} is bonded to phenyl, and the N atom of L^{U} is bonded to Q′.

126. A compound according to any one of aspects 115 and 122 to 125, wherein U is a CRBN ubiquitin ligase binding moiety of formula (U4), wherein R^{U15} and R^{U16} are H, and two of R^{U12}, R^{U13} and R^{U14} are H.

127. A compound according to any one of aspects 115 and 122 to 125, wherein U is a CRBN ubiquitin ligase binding moiety of formula (U4), wherein R^{U13}, R^{U14} and R^{U15} are H, R^{U12} is a single bond to LINK, and R^{U16} is selected from H, F and Me.

128. A compound according to aspect 115, wherein U is a CRBN ubiquitin ligase binding moiety of formula (U5), wherein three of R^{U18}, R^{U19}, R^{U20} and R^{U21} are H.

129. A compound according to any one of the preceding aspects, wherein the N atom of ring Hy which is substituted by R¹ is adjacent to the C atom of ring Hy that is bonded to ring A.

130. A compound according to any one of the preceding aspects, wherein no more than two of Y¹, Y² and Y³ are N.

131. A compound according to any one of the preceding aspects, wherein Z¹ is —C(R⁴)— and Z³ is —N(H)—.

132. A compound according to any one of the preceding aspects, wherein Z¹ is —C(R⁴)—, Z³ is —N(H)—, Y¹ is N, Y² is —C(R⁶)— and Y³ is —C(R⁵)—.

133. A compound according to any one of the preceding aspects, wherein X is a bond, —N(Me)-, O or —CH₂—.

134. A compound according to any one of the preceding aspects, wherein X is a bond.

135. A compound according to any one of the preceding aspects, wherein M is of formula (II):

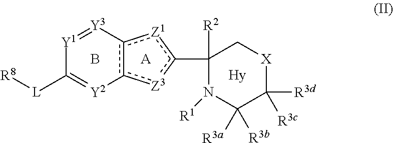

wherein:
Z¹, Z³, Y¹, Y², Y³, R¹, R², R⁸, X and L are as defined in any one of the preceding aspects;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)—,
then neither R^{3c} nor R^{3d} are halo;
wherein at least two of R^{3a}, R^{3b}, R^{3c} and R^{3d} are H; and
wherein M is bonded to LINK via a C or N atom within group R⁸ such that a hydrogen atom on
the C or N atom within group R⁸ is replaced with a bond to LINK.

136. A compound according to any one of the preceding aspects, wherein R⁴, R⁵ and R⁶ are H.

137. A compound according to any one of aspects 101 to 135, wherein one of R⁴, R⁵ and R⁶ is not H.

138. A compound according to aspect 137, wherein one of R⁴, R⁵ and R⁶ is selected from CN, fluoro, chloro, methoxy, methyl and trifluoromethyl, and the rest are H, with the proviso that R⁵ and R⁶ are not CN.

139. A compound according to any one of aspects 101 to 128, wherein M is of formula (III):

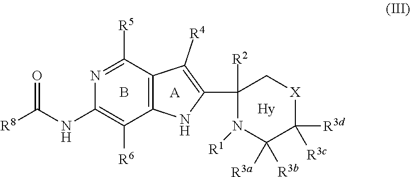

wherein:
R¹ is H or C₁₋₄ alkyl which is itself unsubstituted or substituted with one C₁₋₄ alkoxy or one, two or three halo, preferably with one C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3a} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring,
with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo, C₁₋₄ alkoxy, and C₁₋₄ alkyl which is itself unsubstituted or substituted by one, two or three halo;
R⁵ and R⁶ are independently selected from H, halo, C₁₋₄ alkoxy, and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
wherein M is bonded to LINK via a C or N atom within group R⁸ such that a hydrogen atom on the C or N atom within group R⁸ is replaced with a bond to LINK.

140. A compound according to aspect 139, wherein the C or N atom within group R⁸ that is bonded to LINK is a C or N atom of the heteroaryl ring of group R⁸, or a C or N atom of the heteroaryl, cycloalkyl, heterocyclyl or phenyl ring of R¹⁰.

141. A compound according to any one of aspects 101 to 140, wherein R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl and cyclopropyl, most preferably cyclopropyl.

142. A compound according to aspect 141, wherein R⁸ is an 8- to 10-membered heteroaryl ring which is unsubstituted or substituted by one or two C₁₋₄ alkyl.

143. A compound according to any one of the preceding aspects, wherein Hy is an unsubstituted pyrrolidine ring, R¹ is methyl, and R² is H.

144. A compound according to aspect 101, wherein the PROTAC has a structure selected from:
1-(9-(2-(2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
rac-1-(8-{[2-(2,6-dioxopiperidin-3-yl)-1,3-dioxoisoindol-4-yl]amino}octyl)-3-methyl-N-[2-(1-methylpyrrolidin-2-yl)-1H-pyrrolo[3,2-c]pyridin-6-yl]indazole-6-carboxamide;
1-(8-{[2-(2,6-dioxopiperidin-3-yl)-1,3-dioxoisoindol-4-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-4-yl]amino}octyl)-3-methyl-N-{2-[(2S)-1-methylpyrrolidin-2-yl]-1H-pyrrolo[3,2-c]pyridin-6-yl}indazole-6-carboxamide;
2-(8-((2-(2,6-dioxopiperidin-3-yl)-1,3-dioxoisoindolin-4-yl)amino)octyl)-3-methyl-N-(2-((R)-1-methylpyrrolidin-2-yl)-1H-pyrrolo[3,2-c]pyridin-6-yl)-2H-indazole-6-carboxamide;
3-[4-({4-[2-(2,6-dioxopiperidin-3-yl)-6-fluoro-1,3-dioxoisoindol-5-yl]piperazin-1-yl}methyl)piperidin-1-yl]-1-methyl-N-{2-[(2R)-1-methylpyrrolidin-2-yl]-1H-pyrrolo[3,2-c]pyridin-6-yl}indazole-5-carboxamide;
1-[1-(3-{[2-(2,6-dioxopiperidin-3-yl)-1,3-dioxoisoindol-4-yl]amino}propyl)piperidin-4-yl]-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-5-yl]oxy}octyl)-3-methyl-N-{2-[(2R)-1-methylpyrrolidin-2-yl]-1H-pyrrolo[3,2-c]pyridin-6-yl}indazole-6-carboxamide;
1-{8-[3-(2,4-dioxo-1,3-diazinan-1-yl)-2-methylphenoxy]octyl}-3-methyl-N-{2-[(2R)-1-methylpyrrolidin-2-yl]-1H-pyrrolo[3,2-c]pyridin-6-yl}indazole-6-carboxamide;
1-{6-[3-(2,4-dioxo-1,3-diazinan-1-yl)-2-methylphenoxy]hexyl}-3-methyl-N-{2-[(2R)-1-methylpyrrolidin-2-yl]-1H-pyrrolo[3,2-c]pyridin-6-yl}indazole-6-carboxamide;
1-{7-[1-(2,6-dioxopiperidin-3-yl)-3-methyl-2-oxo-1,3-benzodiazol-4-yl]hept-6-yn-1-yl}-3-methyl-N-{2-[(2R)-1-methylpyrrolidin-2-yl]-1H-pyrrolo[3,2-c]pyridin-6-yl}indazole-6-carboxamide;
1-(8-((2-(2,6-dioxopiperidin-3-yl)-1,3-dioxoisoindolin-4-yl)oxy)octyl)-3-methyl-N-(2-((R)-1-methylpyrrolidin-2-yl)-1H-pyrrolo[3,2-c]pyridin-6-yl)-1H-indazole-6-carboxamide;
1-(3-((2-(2,6-dioxopiperidin-3-yl)-1,3-dioxoisoindolin-4-yl)amino)propyl)-3-methyl-N-(2-((R)-1-methylpyrrolidin-2-yl)-1H-pyrrolo[3,2-c]pyridin-6-yl)-1H-indazole-6-carboxamide;
1-(8-{[2-(2,6-dioxopiperidin-3-yl)-1,3-dioxoisoindol-5-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-5-yl]amino}octyl)-3-methyl-N-{2-[(2S)-1-methylpyrrolidin-2-yl]-1H-pyrrolo[3,2-c]pyridin-6-yl}indazole-6-carboxamide;
1-(8-{[2-(2,6-dioxopiperidin-3-yl)-1-oxo-3H-isoindol-4-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1-oxo-3H-isoindol-4-yl]amino}octyl)-3-methyl-N-{2-[(2S)-1-methylpyrrolidin-2-yl]-1H-pyrrolo[3,2-c]pyridin-6-yl}indazole-6-carboxamide;
N-(3-chloro-2-((R)-1-methylpyrrolidin-2-yl)-1H-pyrrolo[3,2-c]pyridin-6-yl)-1-(8-((2-(2,6-dioxopiperidin-3-yl)-1,3-dioxoisoindolin-4-yl)amino)octyl)-3-methyl-1H-indazole-6-carboxamide;
1-(4-{4-[2-(2,6-dioxopiperidin-3-yl)-1,3-dioxoisoindol-5-yl]piperazin-1-yl}butyl)-3-methyl-N-{2-[(2R)-1-methylpyrrolidin-2-yl]-1H-pyrrolo[3,2-c]pyridin-6-yl}indazole-6-carboxamide;
1-(4-{4-[2-(2,6-dioxopiperidin-3-yl)-1,3-dioxoisoindol-5-yl]piperazin-1-yl}butyl)-3-methyl-N-{2-[(2S)-1-methylpyrrolidin-2-yl]-1H-pyrrolo[3,2-c]pyridin-6-yl}indazole-6-carboxamide;
1-(4-((2-(2,6-dioxopiperidin-3-yl)-1,3-dioxoisoindolin-4-yl)amino)butyl)-3-methyl-N-(2-((R)-1-methylpyrrolidin-2-yl)-1H-pyrrolo[3,2-c]pyridin-6-yl)-1H-indazole-6-carboxamide;
1-(6-{[2-(2,6-dioxopiperidin-3-yl)-1,3-dioxoisoindol-4-yl]amino}hexyl)-3-methyl-N-{2-[(2R)-1-methylpyrrolidin-2-yl]-1H-pyrrolo[3,2-c]pyridin-6-yl}indazole-6-carboxamide;
1-(5-(4-(2-(2,6-dioxopiperidin-3-yl)-6-fluoro-1,3-dioxoisoindolin-5-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide;
1-(2-(2-(4-(2-(2,6-dioxopiperidin-3-yl)-6-fluoro-1,3-dioxoisoindolin-5-yl)piperazin-1-yl)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(2-(2-(2-((2-(2,6-dioxopiperidin-3-yl)-1,3-dioxoisoindolin-4-yl)amino)ethoxy)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(2-(2-((2-(2,6-dioxopiperidin-3-yl)-1,3-dioxoisoindolin-4yl)amino)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(3-(1-(2-(2,6-dioxopiperidin-3-yl)-1,3-dioxoisoindolin-4-yl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide;
1-(3-(1-(4-((2,6-dioxopiperidin-3-yl)carbamoyl)-2-fluorophenyl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide;
1-(5-(1-(2-(2,6-dioxopiperidin-3-yl)-1,3-dioxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide;
1-(7-(2-(2,6-dioxopiperidin-3-yl)-1-oxoisoindolin-4-yl) hept-6-yn-1-yl)-3-methyl-N-(2-((R)-1-methylpyrrolidin-2-yl)-1H-pyrrolo [3,2-c]pyridin-6-yl)-1H-indazole-6-carboxamide;
1-(3-((3-(2-(2,6-dioxopiperidin-3-yl)-1-oxoisoindolin-4-yl)prop-2-yn-1-yl)oxy)propyl)-3-methyl-N-(2-((R)-1-methylpyrrolidin-2-yl)-1H-pyrrolo[3,2-c]pyridin-6-yl)-1H-indazole-6-carboxamide;
1-(5-(2-(2,6-dioxopiperidin-3-yl)-1-oxoisoindolin-4-yl)pent-4-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(2-(2,6-dioxopiperidin-3-yl)-1,3-dioxoisoindolin-4-yl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(3-((2,6-dioxopiperidin-3-yl)carbamoyl)-2-fluorophenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(3-((2,6-dioxopiperidin-3-yl)carbamoyl)-2-methylphenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(8-((2-(2,6-dioxopiperidin-3-yl)-1,3-dioxoisoindolin-4-yl)amino)octyl)-N-(2-((R)-1-methylpyrrolidin-2-yl)-1H-pyrrolo[3,2-c]pyridin-6-yl)-1H-indazole-5-carboxamide;
1-(8-((2-(2,6-dioxopiperidin-3-yl)-1,3-dioxoisoindolin-4-yl)amino)octyl)-N-(2-((S)-1-methylpyrrolidin-2-yl)-1H-pyrrolo[3,2-c]pyridin-6-yl)-1H-indazole-5-carboxamide;
1-(9-(2-(2,6-dioxopiperidin-3-yl)-1-oxoisoindolin-4-yl)non-8-yn-1-yl)-N-(2-((R)-1-methylpyrrolidin-2-yl)-1H-pyrrolo[3,2-c]pyridin-6-yl)-1H-indazole-5-carboxamide;
1-(9-(1-(2,6-dioxopiperidin-3-yl)-3-methyl-2-oxo-2,3-dihydro-1H-benzo[d]imidazol-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-((E)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-((Z)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-(9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-(9-(6-(((S)-2,6-dioxopiperidin-3-yl)carbamoyl)pyridin-3-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(6-(((S)-2,6-dioxopiperidin-3-yl)carbamoyl)pyridin-2-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(((S)-2,6-dioxopiperidin-3-yl)carbamoyl)pyridin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(2-((R)-1-(methyl-d3)pyrrolidin-2-yl)-1H-pyrrolo[3,2-c]pyridin-6-yl)-1H-indazole-6-carboxamide;
1-(8-(2-(2,6-dioxopiperidin-3-yl)-1-oxoisoindolin-4-yl)oct-7-yn-1-yl)-3-methyl-N-(2-((R)-1-methylpyrrolidin-2-yl)-1H-pyrrolo[3,2-c]pyridin-6-yl)-1H-indazole-6-carboxamide;
1-(6-(2-(2,6-dioxopiperidin-3-yl)-1-oxoisoindolin-4-yl)hex-5-yn-1-yl)-3-methyl-N-(2-((R)-1-methylpyrrolidin-2-yl)-1H-pyrrolo[3,2-c]pyridin-6-yl)-1H-indazole-6-carboxamide;
1-(5-(4-(2-(2,6-dioxopiperidin-3-yl)-1,3-dioxoisoindolin-4-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-N-(2-((R)-1-ethylpyrrolidin-2-yl)-1H-pyrrolo[3,2-c]pyridin-6-yl)-3-methyl-1H-indazole-6-carboxamide;
1-(5-((3-(2-(2,6-dioxopiperidin-3-yl)-1-oxoisoindolin-4-yl)prop-2-yn-1-yl)oxy)pentyl)-3-methyl-N-(2-((R)-1-methylpyrrolidin-2-yl)-1H-pyrrolo[3,2-c]pyridin-6-yl)-1H-indazole-6-carboxamide;
1-(2-((6-(2-(2,6-dioxopiperidin-3-yl)-1-oxoisoindolin-4-yl)hex-5-yn-1-yl)oxy)ethyl)-3-methyl-N-(2-((R)-1-methylpyrrolidin-2-yl)-1H-pyrrolo[3,2-c]pyridin-6-yl)-1H-indazole-6-carboxamide;
1-(9-(3-(((S)-2,6-dioxopiperidin-3-yl)carbamoyl)-2-fluorophenyl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-7-fluoro-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
N-(3-chloro-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide;
1-(10-(2-(2,6-dioxopiperidin-3-yl)-1-oxoisoindolin-4-yl) dec-9-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrol[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(2-((R)-5-methyl-5-azaspiro[2.4]heptan-6-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(3-methyl-2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
3-cyclopropyl-1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrolo [3,2-c]pyridin-6-yl)-1H-indazole-6-carboxamide;
1-(9-(2-(2,6-dioxopiperidin-3-yl)-6-fluoro-1-oxoisoindolin-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(3-(2,6-dioxopiperidin-3-yl)-2-fluorophenyl) non-8-yn-1-yl)-3-methyl-N-(2-((R)-1-methylpyrrolidin-2-yl)-1H-pyrrolo [3,2-c]pyridin-6-yl)-1H-indazole-6-carboxamide;
1-(9-(6-(2,6-dioxopiperidin-3-yl) pyridin-2-yl) non-8-yn-1-yl)-3-methyl-N-(2-((R)-1-methylpyrrolidin-2-yl)-1H-pyrrolo [3,2-c]pyridin-6-yl)-1H-indazole-6-carboxamide;
(R)-1-(9-(3-(2,4-dioxotetrahydropyrimidin-1 (2H)-yl)-2-fluorophenyl) non-8-yn-1-yl)-3-methyl-N-(2-(1-methylpyrrolidin-2-yl)-1H-pyrrolo [3,2-c]pyridin-6-yl)-1H-indazole-6-carboxamide;
(R)-1-(9-(6-(2,4-dioxotetrahydropyrimidin-1 (2H)-yl)pyridin-2-yl)non-8-yn-1-yl)-3-methyl-N-(2-(1-methylpyrrolidin-2-yl)-1H-pyrrolo[3,2-c]pyridin-6-yl)-1H-indazole-6-carboxamide;
(R)-1-(9-(3-(2,4-dioxotetrahydropyrimidin-1 (2H)-yl)-5-fluoro-1-methyl-1H-indazol-6-yl)non-8-yn-1-yl)-3-methyl-N-(2-(1-methylpyrrolidin-2-yl)-1H-pyrrolo[3,2-c]pyridin-6-yl)-1H-indazole-6-carboxamide;
N-(3-cyano-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide;
N-(3-chloro-2-((R)-1-methylpyrrolidin-2-yl)-1H-pyrrolo[3,2-c]pyridin-6-yl)-1-(9-(2-(2,6-dioxopiperidin-3-yl)-7-fluoro-1-oxoisoindolin-4-yl)non-8-yn-1-yl)-3-methyl-1H-indazole-6-carboxamide;
1-(1-(3-(2-(2,6-dioxopiperidin-3-yl)-1-oxoisoindolin-4-yl)prop-2-yn-1-yl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-6-fluoro-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-1,3-dioxoisoindolin-4-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(3-(2-(2,6-dioxopiperidin-3-yl)-1-oxoisoindolin-4-yl)prop-2-yn-1-yl)-3-methyl-N-(2-((R)-1-methylpyrrolidin-2-yl)-1H-pyrrolo[3,2-c]pyridin-6-yl)-1H-indazole-6-carboxamide;
1-(6-(1-(6-(((S)-2,6-dioxopiperidin-3-yl)carbamoyl)pyridin-2-yl)piperidin-4-yl)hexyl)-3-methyl-N-(2-((R)-1-methylpyrrolidin-2-yl)-1H-pyrrolo[3,2-c]pyridin-6-yl)-1H-indazole-6-carboxamide;
1-(4-(5-(2-(2,6-dioxopiperidin-3-yl)-1-oxoisoindolin-4-yl) pent-4-yn-1-yl) cyclohexyl)-3-methyl-N-(2-((R)-1-methylpyrrolidin-2-yl)-1H-pyrrolo[3,2-c]pyridin-6-yl)-1H-indazole-6-carboxamide;
1-(9-(2-((rel-S)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-((rel-R)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
N-(3-chloro-2-((R)-1-methylpyrrolidin-2-yl)-1H-pyrrolo[3,2-c]pyridin-6-yl)-1-(9-(2-((rel-S)-2,6-dioxopiperidin-3-yl)-1-oxoisoindolin-4-yl)non-8-yn-1-yl)-3-methyl-1H-indazole-6-carboxamide;
N-(3-chloro-2-((R)-1-methylpyrrolidin-2-yl)-1H-pyrrolo[3,2-c]pyridin-6-yl)-1-(9-(2-((rel-R)-2,6-dioxopiperidin-3-yl)-1-oxoisoindolin-4-yl)non-8-yn-1-yl)-3-methyl-1H-indazole-6-carboxamide;
1-(9-(2-((rel-S)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
1-(9-(2-((rel-R)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
1-((1-(2-(2,6-dioxopiperidin-3-yl)-1,3-dioxoisoindolin-5-yl)piperidin-4-yl) methyl)-3-methyl-N-(2-((R)-1-methylpyrrolidin-2-yl)-1H-pyrrolo[3,2-c]pyridin-6-yl)-1H-indazole-6-carboxamide;
1-(5-(1-(2-(2,6-dioxopiperidin-3-yl)-1-oxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide;
1-((1-(2-(2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide;
1-((1-(2-((rel-S)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide;
1-((1-(2-((rel-R)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide; and
1-(5-(1-(2-(2,6-dioxopiperidin-3-yl)-1,3-dioxoisoindolin-5-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide.

145. A compound according to any one of aspects 101 to 128, wherein M is of formula (IV):

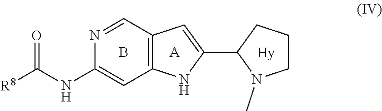

wherein R⁸ is an 8- to 10-membered heteroaryl ring, the group R⁸ being unsubstituted or substituted by one, two or three substituents independently selected from halo, C₁₋₄ alkyl and R¹⁰;
R¹⁰ is a 3-membered cycloalkyl ring;
wherein M is bonded to LINK via a C or N atom within group R⁸ such that a hydrogen atom on the C or N atom within group R⁸ is replaced with a bond to LINK.

146. A compound according to any one of aspects 101 to 145, wherein R⁸ is indazolyl.

147. A compound according to any one of the preceding aspects, wherein the group R⁸ is unsubstituted or substituted by one cyclopropyl group or one or two methyl groups.

148. A compound according to any one of the preceding aspects, wherein the group R⁸ is an indazolyl group which is unsubstituted or substituted by one or two methyl groups.

149. A compound according to any one of aspects 148 to 150, wherein R⁸ is selected from one of the following structures:

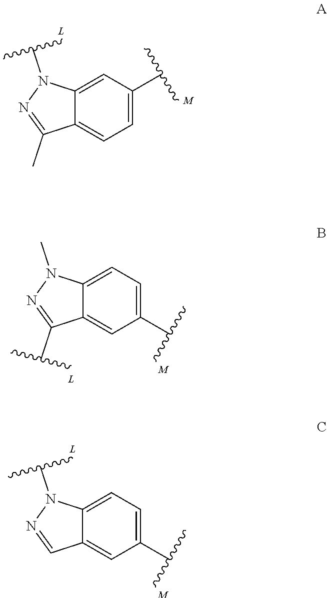

wherein:

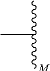

represents the point of attachment of R⁸ to the rest of moiety M;

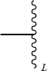

represents the point of attachment of R⁸ to LINK.

150. A compound according to any one of aspects 148 to 149, wherein R⁸ is selected from one of the following structures:

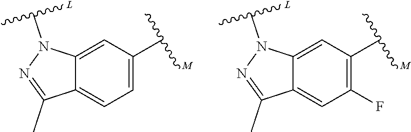

wherein:

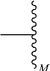

represents the point of attachment of R⁸ to the rest of moiety M;

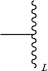

represents the point of attachment of R⁸ to LINK.

151. A compound according to any one of aspects 101 to 135 and 139 to 143, wherein at least one of R⁴, R⁵ and R⁶ is not H.

152. A compound according to any one of aspects 101 to 151, wherein the bond from ring Hy to ring A is in the “up” position and the bond from ring Hy to R² is in the “down” position relative to ring Hy, as depicted below:

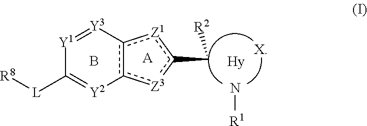

153. A compound according to any one of aspects 101 to 152, wherein the E3 ubiquitin ligase binding moiety (U) has the following structure:

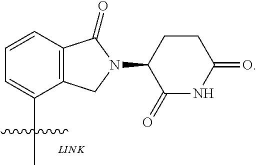 [SMILES: *c1cccc2c1CN([C@H]1CCC(=O)NC1=O)C2=O]

154. A pharmaceutical composition which comprises a compound as defined in any one of aspects 101 to 153 and a pharmaceutically acceptable carrier or diluent.

155. A compound as defined in any one of aspects 101 to 153 for use in the treatment of the human or animal body by therapy.

156. A compound as defined in any one of aspects 101 to 153 for use in the treatment of cancer.

157. The compound for use according to aspect 156, wherein the cancer is a transcriptionally addicted cancer, such as breast cancer, acute leukemia, chronic leukemia, prostate cancer, bladder cancer, cholangiocarcinoma, colon adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, pheochromocytoma and paraganglioma, rectum adenocarcinoma, stomach adenocarcinoma, uterine corpus endometrial carcinoma, cervical squamous cell carcinoma and endocervical adenocarcinoma, lung squamous cell carcinoma, neuroblastoma, midline glioma or sarcoma, preferably neuroblastoma or midline glioma.

158. The compound for use according to aspect 156, wherein the cancer is acute myeloid leukaemia (AML), myelodysplastic syndrome (MDS), myeloproliferative neoplasm (MPN) or acute lymphoblastic leukaemia (ALL).

159. The compound for use according to aspect 158, wherein the cancer is mixed-lineage leukaemia (MLL) rearranged (also known as KMT2Ar) acute leukemia (AML and ALL).

160. The compound for use according to aspect 158, wherein the cancer is NPM1 mutant acute leukemia.

161. The compound for use according to aspect 158, wherein the cancer is RUNX1-fusion acute leukemia.

162. The compound for use according to aspect 158, wherein the cancer is E2A-fusion acute leukemia.

163. The compound for use according to aspect 158, wherein the cancer is PML-fusion acute leukemia.

164. The compound for use according to aspect 158, wherein the cancer is NUP98-fusion acute leukemia.

Alternatively, the invention provides compounds according to the following embodiments.

1. A compound which is a bicyclic compound of formula (I′) or a pharmaceutically acceptable salt thereof:

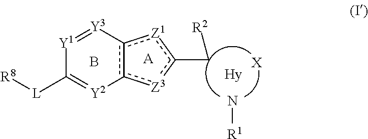

wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)-, O, S, —S(O)₂—, —S(O)(NR¹¹)—, or —C(R¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
at least one of R⁴, R⁵ and R⁶ is not H;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
each R¹¹ is independently selected from H, C₁₋₄ alkyl, and C₁₋₄ cycloalkyl.

2. A compound according to embodiment 1, wherein one of R⁴, R⁵ and R⁶ is selected from halo, C₁₋₄ alkoxy, and C₁₋₄ alkyl which is itself unsubstituted or substituted by one, two or three halo, and the rest are H.

3. A compound according to embodiment 1 or 2 wherein R⁴ is selected from halo, C₁₋₄ alkoxy, and C₁₋₄ alkyl which is itself unsubstituted or substituted by one, two or three halo.

4. A compound according to embodiment 3, wherein R⁴ is selected from halo, and C₁₋₄ alkyl which is itself substituted by one, two or three halo.

5. A compound according to embodiment 4, wherein R⁴ is selected from F, Cl and CF₃.

6. A compound according to any one of embodiments 1 to 5, wherein R⁵ is C₁₋₄ alkyl.

7. A compound according to embodiment 6, wherein R⁵ is methyl.

8. A compound according to any one of embodiments 1 to 7, wherein R¹ is H, C₁₋₄ cycloalkyl, unsubstituted C₂0.4 alkyl, or C₁₋₄ alkyl which is itself substituted with one C₁₋₄ alkoxy or one, two or three halo.

9. A compound according to any one of embodiments 1 to 7, wherein R¹ is C₁₋₄ alkyl.

10. A compound according to embodiment 9, wherein R¹ is methyl.

11. A compound according to embodiment 8, wherein R¹ is H or C₂₋₄ alkyl.

12. A compound according to any one of embodiments 1 to 11, wherein R² is H.

13. A compound according to any one of embodiments 1 to 12, wherein the N atom of ring Hy which is substituted by R¹ is adjacent to the C atom of ring Hy that is bonded to ring A.

14. A compound according to any one of embodiments 1 to 13, wherein no more than two of Y¹, Y² and Y³ are N.

15. A compound according to any one of embodiments 1 to 14, wherein Z¹ is —C(R⁴)— and Z³ is —N(H)—.

16. A compound according to any one of embodiments 1 to 15, wherein Y² is —C(R⁶)— and Y³ is —C(R⁵)—.

17. A compound according to any one of embodiments 1 to 16, wherein X is a bond, —N(Me)-, O or —CH₂—.

18. A compound according to embodiment 17, wherein X is a bond.

19. A compound according to any one of embodiments 1 to 18, which is a bicyclic compound of formula (II′) or a pharmaceutically acceptable salt thereof:

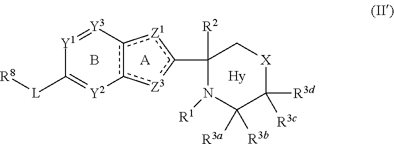

wherein:
Z¹, Z³, Y¹, Y², Y³, R¹, R², R⁸, X and L are as defined in any one of embodiments 1 to 18; R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, with the proviso that when X is —N(R¹¹)—, O, S, —S(O)₂— or —S(O)(NR¹¹)—, then neither R^{3c} nor R^{3d} are halo;
or
R^{3a} and R^{3c} are H, and R^{3b} and R^{3d} form, together with the C atoms to which they are attached, a C₅₋₆ cycloalkyl ring;
wherein at least two of R^{3a}, R^{3b}, R^{3c} and R^{3d} are H.

20. A compound according to any one of embodiments 1 to 19, which is a bicyclic compound of formula (III′) or a pharmaceutically acceptable salt thereof:

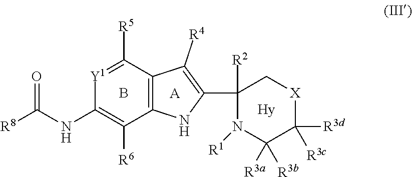

wherein:
X, Y¹, R¹, R², R⁴, R⁵, R⁶, R^{3a}, R^{3b}, R^{3c}, R^{3a} and R⁸ are as defined in any one of embodiments 1 to 19.

21. A compound according to any one of embodiments 1 to 20, wherein R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3a} form, together with the C atom to which they are attached, a C₃₋₆ cycloalkyl ring, with the proviso that when X is —N(R¹¹)—, O, S, —S(O)₂— or —S(O)(NR¹¹)—, then neither R^{3c} nor R^{3d} are halo; or
R^{3a} and R^{3c} are H, and R^{3b} and R^{3d} form, together with the C atoms to which they are attached, a C₅₋₆ cycloalkyl ring;
wherein at least two of R^{3a}, R^{3b}, R^{3c} and R^{3d} are H;

22. A compound according to embodiment 21, wherein R^{3a}, R^{3b}, R^{3c} and R^{3d} are H.

23. A compound according to any one of embodiments 1 to 22, wherein Hy is a pyrrolidine ring, R¹ is methyl, and R² is H.

24. A compound according to any one of embodiments 1 to 23, wherein the group R⁸ is unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo.

25. A compound according to any one of embodiments 1 to 24, wherein the group R⁸ is unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one R⁹, preferably wherein R¹⁰ is selected from cyclopropyl, cyclobutyl, cyclopentyl, cyclohexyl, imidazolyl, oxadiazolyl, oxetanyl, phenyl, pyrazinyl, pyrazolyl, pyridazinyl, pyridinyl, pyrimidinyl, pyrrolidinyl, thiazinyl, thiazolyl and triazolyl, more preferably pyrazolyl.

26. A compound according to any one of embodiments 1 to 25, wherein R⁸ is an 8- to 10-membered heteroaryl ring containing one to three nitrogen atoms, the group R⁸ being unsubstituted or substituted by one, two or three substituents as defined in claim 25.

27. A compound according to any one of embodiments 1 to 26, wherein R⁸ is indazolyl which is unsubstituted or substituted by one, two or three substituents as defined in claim 25.

28. A compound according to embodiment 27, wherein R⁸ is indazolyl which is unsubstituted or substituted by one or two C₁₋₄ alkyl.

29. A compound according to embodiment 28, wherein R⁸ is indazolyl which is unsubstituted or substituted by one or two methyl.

29. A compound according to embodiment 30, wherein R⁸ is selected from one of the following structures:

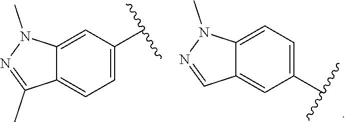 [SMILES: *c1ccc2c(C)nn(C)c2c1 ; *c1ccc2c(cnn2C)c1]

30. A compound according to embodiment 1 which is selected from:
N-{3-chloro-2-[(2R)-1-methylpyrrolidin-2-yl]-1H-pyrrolo[3,2-c]pyridin-6-yl}-1,3-dimethylindazole-6-carboxamide;
N-{3-chloro-2-[(2S)-1-methylpyrrolidin-2-yl]-1H-pyrrolo[3,2-c]pyridin-6-yl}-1,3-dimethylindazole-6-carboxamide;
(R)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide;
(S)-1,3-dimethyl-N-(2-(1-methylpyrrolidin-2-yl)-3-(trifluoromethyl)-1H-pyrrolo[3,2-c]pyridin-6-yl)-1H-indazole-6-carboxamide;
N-{3-fluoro-2-[(rel-2S)-1-methylpyrrolidin-2-yl]-1H-pyrrolo[3,2-c]pyridin-6-yl}-1-methylindazole-5-carboxamide;
N-{3-fluoro-2-[(rel-2R)-1-methylpyrrolidin-2-yl]-1H-pyrrolo[3,2-c]pyridin-6-yl}-1-methylindazole-5-carboxamide;
N-{3-fluoro-2-[(rel-2R)-1-methylpyrrolidin-2-yl]-1H-pyrrolo[3,2-c]pyridin-6-yl}-1,3-dimethylindazole-6-carboxamide;
N-{3-fluoro-2-[(rel-2S)-1-methylpyrrolidin-2-yl]-1H-pyrrolo[3,2-c]pyridin-6-yl}-1,3-dimethylindazole-6-carboxamide;
1,3-dimethyl-N-{3-methyl-2-[(rel-2R)-1-methylpyrrolidin-2-yl]-1H-pyrrolo[3,2-c]pyridin-6-yl}indazole-6-carboxamide;
1,3-dimethyl-N-{3-methyl-2-[(rel-2S)-1-methylpyrrolidin-2-yl]-1H-pyrrolo[3,2-c]pyridin-6-yl}indazole-6-carboxamide;
1,3-dimethyl-N-{4-methyl-2-[(rel-2R)-1-methylpyrrolidin-2-yl]-1H-pyrrolo[3,2-c]pyridin-6-yl}indazole-6-carboxamide;
1,3-dimethyl-N-{4-methyl-2-[(rel-2S)-1-methylpyrrolidin-2-yl]-1H-pyrrolo[3,2-c]pyridin-6-yl}indazole-6-carboxamide; and
(R)—N-(3-cyano-2-(1-methylpyrrolidin-2-yl)-1H-pyrrolo[3,2-c]pyridin-6-yl)-1,3-dimethyl-1H-indazole-6-carboxamide;
   and the pharmaceutically acceptable salts thereof.

31. A pharmaceutical composition which comprises a compound as defined in any one of embodiments 1 to 30 and a pharmaceutically acceptable carrier or diluent.

32. A compound as defined in any one of embodiments 1 to 30 for use in the treatment of the human or animal body by therapy.

33. A compound as defined in any one of embodiments 1 to 30 for use in the treatment of cancer.

34. The compound for use according to embodiment 33, wherein the cancer is a transcriptionally addicted cancer, such as breast cancer, acute leukemia, prostate cancer, bladder cancer, cholangiocarcinoma, colon adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, pheochromocytoma and paraganglioma, rectum adenocarcinoma, stomach adenocarcinoma, uterine corpus endometrial carcinoma, cervical squamous cell carcinoma and endocervical adenocarcinoma, lung squamous cell carcinoma, neuroblastoma, midline glioma or sarcoma, preferably neuroblastoma or midline glioma.

35. The compound for use according to embodiment 33, wherein the cancer is acute myeloid leukaemia (AML), myelodysplastic syndrome (MDS), myeloproliferative neoplasm (MPN) or acute lymphoblastic leukaemia (ALL).

36. The compound for use according to embodiment 35, wherein the cancer is mixed-lineage leukaemia (MLL) or rearranged acute leukemia (AML and ALL).

37. The compound for use according to embodiment 35, wherein the cancer is NPM1 mutant acute leukemia.

1. A compound which is a Proteolysis Targeting Chimera (PROTAC) or a pharmaceutically acceptable salt thereof, wherein the PROTAC has the structure: 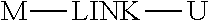
wherein U is an E3 ubiquitin ligase binding moiety, LINK is a moiety that covalently links M and U, and M is an MLLT1 and/or MLLT3 binder of formula (I): 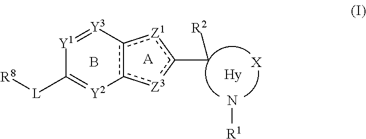
wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C3-6 cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
each R¹¹ is independently selected from H, C₁₋₄ alkyl, and C₁₋₄ cycloalkyl;
wherein M is bonded to LINK via a C or N atom within group R⁸ such that a hydrogen atom on the C or N atom within group R⁸ is replaced with a bond to LINK.

2. A compound according to claim 1, wherein LINK is (a) a single bond, or (b) a chemical linker group represented by formula (L): 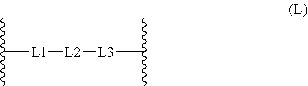
wherein:
LINK is attached to M via L1;
LINK is attached to U via L3;
L1 and L3 are each independently selected from a single bond, —N(R′)—, —C(O)N(R′)—, —O—, —N(R′)C(O)—, —C(O)—, —S(O₂)N(R′)—, —N(R′)S(O₂)—, —(C₁₋₆ alkylene)-, ethynylene, —(C₂₋₆ alkenylene)-, —C═C═C—, phenylene, a divalent 3- to 6-membered cycloalkyl ring and a divalent 4- to 7-membered heterocyclyl ring, the phenylene, cycloalkyl and heterocyclyl rings being unsubstituted or substituted by one or two C₁₋₄ alkyl;
L2 is represented by the formula -(L4)ₘ-;
each L4 is independently a unit of formula: 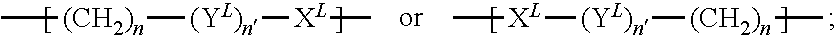
each X^{L} is independently selected from a single bond, —N(R′)—, —O—, —C(O)—, —S—, —SO—, —SO₂—, —C(R″)═C(R″)— and —C≡C—;
each Y^{L} is independently selected from a divalent 3- to 6-membered cycloalkyl ring, a divalent 4- to 7-membered heterocyclyl ring, or one of the following structures: 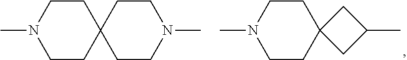
the cycloalkyl and heterocyclyl rings being unsubstituted or substituted by one or two C₁₋₄ alkyl;
n is selected from 0 to 4;
n′ is selected from 0 or 1;
n+n′>1;
m is selected from 1 to 30;
each R′ is independently selected from H and C₁₋₄ alkyl; and
each R¹¹ is independently selected from H and halo.

3. A compound according to claim 1 or 2, wherein U is:
(a) a CRBN E3 ubiquitin ligase binding moiety of formula (U1): 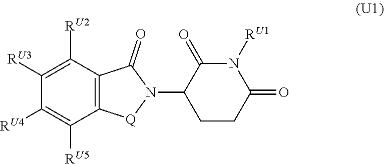
wherein:
R^{U1} is H;
Q is selected from —CH(R^{U6})—, —N(R^{U6})—, —O—, —C(O)—, —NH—CH(R^{U6}),
N═C(R^{U6})—, or —N═N—;
R^{U6} is H or C₁₋₄ alkyl;
R^{U2} and R^{U5} are each independently selected from H, halogen, C₁₋₄ alkyl, NH₂, NO₂, OH, COOH, CN, CF₃, and a single bond to LINK;
R^{U3} and R^{U4} are each independently selected from H, halogen, C₁₋₄ alkyl, NH₂, NO₂, OH, COOH, CN, CF₃, and a single bond to LINK;
wherein one and only one of R^{U2}, R^{U3}, R^{U4} and RUS is a single bond to LINK;
(b) a CRBN E3 ubiquitin ligase binding moiety of formula (U4): 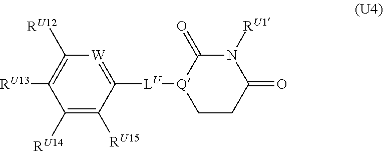
wherein:
R^{U1′} is H;
W is N or CR^{U16};
Q′ is N and L^{U} is a single bond, or Q′ is CH and L^{U} is selected from a single bond or —C(O)N(H)—, wherein either (i) the C atom of L^{U} is bonded to phenyl, and the N atom of L^{U} is bonded to Q′; or (ii) the C atom of L^{U} is bonded to Q′, and the N atom of L^{U} is bonded to phenyl;
R^{U12}, R^{U13} and R^{U14} are each independently selected from H, halogen, C₁₋₄ alkyl, NH₂, NO₂, OH, COOH, CN, CF₃, and a single bond to LINK;
wherein one and only one of R^{U12}, R^{U13} and R^{U14} is a single bond to LINK;
R^{U15} and R^{U16} are each independently selected from H, halogen, C₁₋₄ alkyl, NH₂, NO₂, OH, COOH, CN and CF₃;
(c) a CRBN E3 ubiquitin ligase binding moiety of formula (U5): 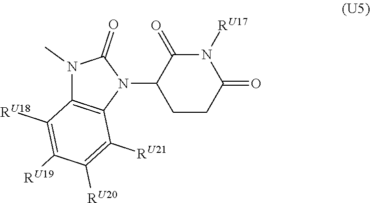
wherein:
R^{U17} is H;
R^{U18}, R^{U19}, R^{U20} and R^{U21} are each independently selected from H, halogen, C₁₋₄ alkyl, NH₂, NO₂, OH, COOH, CN, CF₃, and a single bond to LINK;
wherein one and only one of R^{U18}, R^{U19}, R^{U20} and R^{U21} is a single bond to LINK; or
(d) a VHL E3 ubiquitin ligase binding moiety of formula (U2): 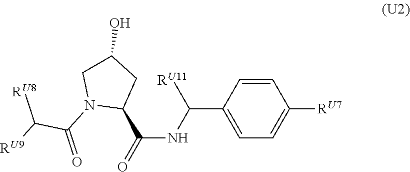
wherein:
R^{U7} is a group selected from phenyl, a 5- to 6-membered heteroaryl ring, a 5- to 6-membered heterocyclyl ring, and a 5- to 6-membered cycloalkyl ring, the group R^{U7} being unsubstituted or substituted by C₁₋₄ alkyl;
R^{U11} is H or C₁₋₄ alkyl;
R^{U8} is selected from C₁₋₄ alkyl, phenyl and a 3- to 8-membered cycloalkyl ring; and
R^{U9} is a single bond to LINK.

4. A compound according to any one of the preceding claims, wherein the N atom of ring Hy which is substituted by R¹ is adjacent to the C atom of ring Hy that is bonded to ring A.

5. A compound according to any one of the preceding claims, wherein Z¹ is —C(R⁴)—, Z³ is —N(H)—, Y¹ is N, Y² is —C(R⁶)— and Y³ is —C(R⁵)—.

6. A compound according to any one of the preceding claims, wherein M is of formula (II): 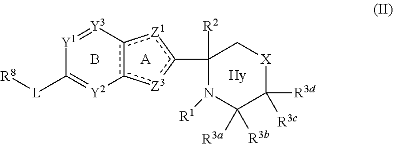
wherein:
Z¹, Z³, Y¹, Y², Y³, R¹, R², R⁸, X and L are as defined in any one of the preceding claims;
R^{3a} and R^{3b} are independently selected from H, C₁₋₄ alkyl, phenyl, a 5- to 6-membered heteroaryl ring and C₁₋₄ alkoxy, or R^{3a} and R^{3b} form, together with the C atom to which they are attached, a C₃₋₆ cycloalkyl ring or a C₃₋₆ heterocycloalkyl ring,
R^{3c} and R^{3d} are independently selected from H, C₁₋₄ alkyl, halo, phenyl, a 5- to 6-membered heteroaryl ring and C₁₋₄ alkoxy, or R^{3c} and R^{3d} form, together with the C atom to which they are attached, a C₃₋₆ cycloalkyl ring or a C₃₋₆ heterocycloalkyl ring, or
R^{3a} and R^{3c} are H, and R^{3b} and R^{3d} form, together with the C atoms to which they are attached, a C₅₋₆ cycloalkyl ring, with the proviso that when X is —N(R¹¹)—, O, S, —S(O)₂— or —S(O)(NR¹¹)—, then neither R^{3c} nor R^{3d} are halo;
wherein at least two of R^{3a}, R^{3b}, R^{3c} and R^{3d} are H; and
wherein M is bonded to LINK via a C or N atom within group R⁸ such that a hydrogen atom on the C or N atom within group R⁸ is replaced with a bond to LINK.

7. A compound according to any one of the preceding claims, wherein M is of formula (III): 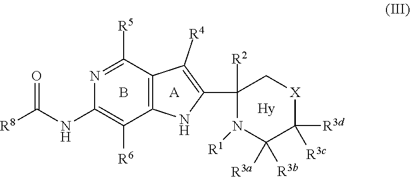
wherein:
R¹ is H or C₁₋₄ alkyl which is itself unsubstituted or substituted with one C₁₋₄ alkoxy or one, two or three halo, preferably with one C₁₋₄ alkoxy;
R² is H or methyl;
R^{3a} and R^{3b} are independently selected from H, C₁₋₄ alkyl, and C₁₋₄ alkoxy, or R^{3a} and R^{3b} form, together with the C atom to which they are attached, a C₃₋₆ cycloalkyl ring,
R^{3c} and R^{3d} are independently selected from H, C₁₋₄ alkyl, halo and C₁₋₄ alkoxy, or R^{3c} and R^{3d} form, together with the C atom to which they are attached, a C₃₋₆ cycloalkyl ring, or
R^{3a} and R^{3c} are H, and R^{3b} and R^{3d} form, together with the C atoms to which they are attached, a C₅₋₆ cycloalkyl ring,
with the proviso that when X is —N(Me)- or O, then neither R^{3c} nor R^{3d} are halo;
wherein at least two of R^{3a}, R^{3b}, R^{3c} and R^{3d} are H;
X is a bond, —N(Me)-, O or —CH₂—;
R⁴ is selected from H, CN, halo, C₁₋₄ alkoxy, and C₁₋₄ alkyl which is itself unsubstituted or substituted by one, two or three halo;
R⁵ and R⁶ are independently selected from H, halo, C₁₋₄ alkoxy, and C₁₋₄ alkyl which is itself unsubstituted or substituted by one, two or three halo;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo; and
wherein M is bonded to LINK via a C or N atom within group R⁸ such that a hydrogen atom on the C or N atom within group R⁸ is replaced with a bond to LINK.

8. A compound according to claim 1, wherein the PROTAC has a structure selected from:
1-(9-(2-(2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
rac-1-(8-{[2-(2,6-dioxopiperidin-3-yl)-1,3-dioxoisoindol-4-yl]amino}octyl)-3-methyl-N-[2-(1-methylpyrrolidin-2-yl)-1H-pyrrolo[3,2-c]pyridin-6-yl]indazole-6-carboxamide;
1-(8-{[2-(2,6-dioxopiperidin-3-yl)-1,3-dioxoisoindol-4-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-4-yl]amino}octyl)-3-methyl-N-{2-[(2S)-1-methylpyrrolidin-2-yl]-1H-pyrrolo[3,2-c]pyridin-6-yl}indazole-6-carboxamide;
2-(8-((2-(2,6-dioxopiperidin-3-yl)-1,3-dioxoisoindolin-4-yl)amino)octyl)-3-methyl-N-(2-((R)-1-methylpyrrolidin-2-yl)-1H-pyrrolo[3,2-c]pyridin-6-yl)-2H-indazole-6-carboxamide;
3-[4-({4-[2-(2,6-dioxopiperidin-3-yl)-6-fluoro-1,3-dioxoisoindol-5-yl]piperazin-1-yl}methyl)piperidin-1-yl]-1-methyl-N-{2-[(2R)-1-methylpyrrolidin-2-yl]-1H-pyrrolo[3,2-c]pyridin-6-yl}indazole-5-carboxamide;
1-[1-(3-{[2-(2,6-dioxopiperidin-3-yl)-1,3-dioxoisoindol-4-yl]amino}propyl)piperidin-4-yl]-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-5-yl]oxy}octyl)-3-methyl-N-{2-[(2R)-1-methylpyrrolidin-2-yl]-1H-pyrrolo[3,2-c]pyridin-6-yl}indazole-6-carboxamide;
1-{8-[3-(2,4-dioxo-1,3-diazinan-1-yl)-2-methylphenoxy]octyl}-3-methyl-N-{2-[(2R)-1-methylpyrrolidin-2-yl]-1H-pyrrolo[3,2-c]pyridin-6-yl}indazole-6-carboxamide;
1-{6-[3-(2,4-dioxo-1,3-diazinan-1-yl)-2-methylphenoxy]hexyl}-3-methyl-N-{2-[(2R)-1-methylpyrrolidin-2-yl]-1H-pyrrolo[3,2-c]pyridin-6-yl}indazole-6-carboxamide;
1-{7-[1-(2,6-dioxopiperidin-3-yl)-3-methyl-2-oxo-1,3-benzodiazol-4-yl]hept-6-yn-1-yl}-3-methyl-N-{2-[(2R)-1-methylpyrrolidin-2-yl]-1H-pyrrolo[3,2-c]pyridin-6-yl}indazole-6-carboxamide;
1-(8-((2-(2,6-dioxopiperidin-3-yl)-1,3-dioxoisoindolin-4-yl)oxy)octyl)-3-methyl-N-(2-((R)-1-methylpyrrolidin-2-yl)-1H-pyrrolo[3,2-c]pyridin-6-yl)-1H-indazole-6-carboxamide;
1-(3-((2-(2,6-dioxopiperidin-3-yl)-1,3-dioxoisoindolin-4-yl)amino)propyl)-3-methyl-N-(2-((R)-1-methylpyrrolidin-2-yl)-1H-pyrrolo[3,2-c]pyridin-6-yl)-1H-indazole-6-carboxamide;
1-(8-{[2-(2,6-dioxopiperidin-3-yl)-1,3-dioxoisoindol-5-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1,3-dioxoisoindol-5-yl]amino}octyl)-3-methyl-N-{2-[(2S)-1-methylpyrrolidin-2-yl]-1H-pyrrolo[3,2-c]pyridin-6-yl}indazole-6-carboxamide;
1-(8-{[2-(2,6-dioxopiperidin-3-yl)-1-oxo-3H-isoindol-4-yl]amino}octyl)-3-methyl-N-{2-[(2R)-1-methylpyrrolidin-2-yl]-1H-pyrrolo[3,2-c]pyridin-6-yl}indazole-6-carboxamide;
1-(8-{[2-(2,6-dioxopiperidin-3-yl)-1-oxo-3H-isoindol-4-yl]amino}octyl)-3-methyl-N-{2-[(2S)-1-methylpyrrolidin-2-yl]-1H-pyrrolo[3,2-c]pyridin-6-yl}indazole-6-carboxamide;
N-(3-chloro-2-((R)-1-methylpyrrolidin-2-yl)-1H-pyrrolo[3,2-c]pyridin-6-yl)-1-(8-((2-(2,6-dioxopiperidin-3-yl)-1,3-dioxoisoindolin-4-yl)amino)octyl)-3-methyl-1H-indazole-6-carboxamide;
1-(4-{4-[2-(2,6-dioxopiperidin-3-yl)-1,3-dioxoisoindol-5-yl]piperazin-1-yl}butyl)-3-methyl-N-{2-[(2R)-1-methylpyrrolidin-2-yl]-1H-pyrrolo[3,2-c]pyridin-6-yl}indazole-6-carboxamide;
1-(4-{4-[2-(2,6-dioxopiperidin-3-yl)-1,3-dioxoisoindol-5-yl]piperazin-1-yl}butyl)-3-methyl-N-{2-[(2S)-1-methylpyrrolidin-2-yl]-1H-pyrrolo[3,2-c]pyridin-6-yl}indazole-6-carboxamide;
1-(4-((2-(2,6-dioxopiperidin-3-yl)-1,3-dioxoisoindolin-4-yl)amino)butyl)-3-methyl-N-(2-((R)-1-methylpyrrolidin-2-yl)-1H-pyrrolo[3,2-c]pyridin-6-yl)-1H-indazole-6-carboxamide;
1-(6-{[2-(2,6-dioxopiperidin-3-yl)-1,3-dioxoisoindol-4-yl]amino}hexyl)-3-methyl-N-{2-[(2R)-1-methylpyrrolidin-2-yl]-1H-pyrrolo[3,2-c]pyridin-6-yl}indazole-6-carboxamide;
1-(5-(4-(2-(2,6-dioxopiperidin-3-yl)-6-fluoro-1,3-dioxoisoindolin-5-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide;
1-(2-(2-(4-(2-(2,6-dioxopiperidin-3-yl)-6-fluoro-1,3-dioxoisoindolin-5-yl)piperazin-1-yl)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(2-(2-(2-((2-(2,6-dioxopiperidin-3-yl)-1,3-dioxoisoindolin-4-yl)amino)ethoxy)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(2-(2-((2-(2,6-dioxopiperidin-3-yl)-1,3-dioxoisoindolin-4yl)amino)ethoxy)ethyl)-3-methyl-N-(2-((R)-1-methylpyrrolidin-2-yl)-1H-pyrrolo[3,2-c]pyridin-6-yl)-1H-indazole-6-carboxamide;
1-(3-(1-(2-(2,6-dioxopiperidin-3-yl)-1,3-dioxoisoindolin-4-yl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide;
1-(3-(1-(4-((2,6-dioxopiperidin-3-yl)carbamoyl)-2-fluorophenyl)piperidin-4-yl)propyl)-3-methyl-N-(2-((R)-1-methylpyrrolidin-2-yl)-1H-pyrrolo[3,2-c]pyridin-6-yl)-1H-indazole-6-carboxamide;
1-(5-(1-(2-(2,6-dioxopiperidin-3-yl)-1,3-dioxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide;
1-(7-(2-(2,6-dioxopiperidin-3-yl)-1-oxoisoindolin-4-yl) hept-6-yn-1-yl)-3-methyl-N-(2-((R)-1-methylpyrrolidin-2-yl)-1H-pyrrolo [3,2-c]pyridin-6-yl)-1H-indazole-6-carboxamide;
1-(3-((3-(2-(2,6-dioxopiperidin-3-yl)-1-oxoisoindolin-4-yl)prop-2-yn-1-yl)oxy)propyl)-3-methyl-N-(2-((R)-1-methylpyrrolidin-2-yl)-1H-pyrrolo[3,2-c]pyridin-6-yl)-1H-indazole-6-carboxamide;
1-(5-(2-(2,6-dioxopiperidin-3-yl)-1-oxoisoindolin-4-yl)pent-4-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(2-(2,6-dioxopiperidin-3-yl)-1,3-dioxoisoindolin-4-yl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(3-((2,6-dioxopiperidin-3-yl)carbamoyl)-2-fluorophenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(7-(3-((2,6-dioxopiperidin-3-yl)carbamoyl)-2-methylphenyl)hept-6-yn-1-yl)-3-methyl-N-(2-((R)-1-methylpyrrolidin-2-yl)-1H-pyrrolo[3,2-c]pyridin-6-yl)-1H-indazole-6-carboxamide;
1-(8-((2-(2,6-dioxopiperidin-3-yl)-1,3-dioxoisoindolin-4-yl)amino)octyl)-N-(2-((R)-1-methylpyrrolidin-2-yl)-1H-pyrrolo[3,2-c]pyridin-6-yl)-1H-indazole-5-carboxamide;
1-(8-((2-(2,6-dioxopiperidin-3-yl)-1,3-dioxoisoindolin-4-yl)amino)octyl)-N-(2-((S)-1-methylpyrrolidin-2-yl)-1H-pyrrolo[3,2-c]pyridin-6-yl)-1H-indazole-5-carboxamide;
1-(9-(2-(2,6-dioxopiperidin-3-yl)-1-oxoisoindolin-4-yl)non-8-yn-1-yl)-N-(2-((R)-1-methylpyrrolidin-2-yl)-1H-pyrrolo[3,2-c]pyridin-6-yl)-1H-indazole-5-carboxamide;
1-(9-(1-(2,6-dioxopiperidin-3-yl)-3-methyl-2-oxo-2,3-dihydro-1H-benzo[d]imidazol-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-((E)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-((Z)-9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-(9-chloro-9-(1-(2,6-dioxopiperidin-3-yl)-3-methyl-2-oxo-2,3-dihydro-1H-benzo[d]imidazol-5-yl)non-8-en-1-yl)-3-methyl-N-(2-((R)-1-methylpyrrolidin-2-yl)-1H-pyrrolo[3,2-c]pyridin-6-yl)-1H-indazole-6-carboxamide;
1-(9-(6-(((S)-2,6-dioxopiperidin-3-yl)carbamoyl)pyridin-3-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(6-(((S)-2,6-dioxopiperidin-3-yl)carbamoyl)pyridin-2-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(((S)-2,6-dioxopiperidin-3-yl)carbamoyl)pyridin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(2-((R)-1-(methyl-d3)pyrrolidin-2-yl)-1H-pyrrolo[3,2-c]pyridin-6-yl)-1H-indazole-6-carboxamide;
1-(8-(2-(2,6-dioxopiperidin-3-yl)-1-oxoisoindolin-4-yl)oct-7-yn-1-yl)-3-methyl-N-(2-((R)-1-methylpyrrolidin-2-yl)-1H-pyrrolo[3,2-c]pyridin-6-yl)-1H-indazole-6-carboxamide;
1-(6-(2-(2,6-dioxopiperidin-3-yl)-1-oxoisoindolin-4-yl)hex-5-yn-1-yl)-3-methyl-N-(2-((R)-1-methylpyrrolidin-2-yl)-1H-pyrrolo[3,2-c]pyridin-6-yl)-1H-indazole-6-carboxamide;
1-(5-(4-(2-(2,6-dioxopiperidin-3-yl)-1,3-dioxoisoindolin-4-yl)piperazin-1-yl)pentyl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-N-(2-((R)-1-ethylpyrrolidin-2-yl)-1H-pyrrolo[3,2-c]pyridin-6-yl)-3-methyl-1H-indazole-6-carboxamide;
1-(5-((3-(2-(2,6-dioxopiperidin-3-yl)-1-oxoisoindolin-4-yl)prop-2-yn-1-yl)oxy)pentyl)-3-methyl-N-(2-((R)-1-methylpyrrolidin-2-yl)-1H-pyrrolo[3,2-c]pyridin-6-yl)-1H-indazole-6-carboxamide;
1-(2-((6-(2-(2,6-dioxopiperidin-3-yl)-1-oxoisoindolin-4-yl)hex-5-yn-1-yl)oxy)ethyl)-3-methyl-N-(2-((R)-1-methylpyrrolidin-2-yl)-1H-pyrrolo[3,2-c]pyridin-6-yl)-1H-indazole-6-carboxamide;
1-(9-(3-(((S)-2,6-dioxopiperidin-3-yl)carbamoyl)-2-fluorophenyl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-7-fluoro-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
N-(3-chloro-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide;
1-(10-(2-(2,6-dioxopiperidin-3-yl)-1-oxoisoindolin-4-yl) dec-9-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrol[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(2-((R)-5-methyl-5-azaspiro[2.4]heptan-6-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-3-methyl-N-(3-methyl-2-((R)-1-methylpyrrolidin-2-yl)-1H-pyrrolo[3,2-c]pyridin-6-yl)-1H-indazole-6-carboxamide;
1-(9-(2-(2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
3-cyclopropyl-1-(9-(2-(2,6-dioxopiperidin-3-yl)-1-oxoisoindolin-4-yl) non-8-yn-1-yl)-N-(2-((R)-1-methylpyrrolidin-2-yl)-1H-pyrrolo [3,2-c]pyridin-6-yl)-1H-indazole-6-carboxamide;
1-(9-(2-(2,6-dioxopiperidin-3-yl)-6-fluoro-1-oxoisoindolin-5-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(3-(2,6-dioxopiperidin-3-yl)-2-fluorophenyl) non-8-yn-1-yl)-3-methyl-N-(2-((R)-1-methylpyrrolidin-2-yl)-1H-pyrrolo [3,2-c]pyridin-6-yl)-1H-indazole-6-carboxamide;
1-(9-(6-(2,6-dioxopiperidin-3-yl) pyridin-2-yl) non-8-yn-1-yl)-3-methyl-N-(2-((R)-1-methylpyrrolidin-2-yl)-1H-pyrrolo [3,2-c]pyridin-6-yl)-1H-indazole-6-carboxamide;
(R)-1-(9-(3-(2,4-dioxotetrahydropyrimidin-1 (2H)-yl)-2-fluorophenyl) non-8-yn-1-yl)-3-methyl-N-(2-(1-methylpyrrolidin-2-yl)-1H-pyrrolo [3,2-c]pyridin-6-yl)-1H-indazole-6-carboxamide;
(R)-1-(9-(6-(2,4-dioxotetrahydropyrimidin-1 (2H)-yl)pyridin-2-yl)non-8-yn-1-yl)-3-methyl-N-(2-(1-methylpyrrolidin-2-yl)-1H-pyrrolo[3,2-c]pyridin-6-yl)-1H-indazole-6-carboxamide;
(R)-1-(9-(3-(2,4-dioxotetrahydropyrimidin-1 (2H)-yl)-5-fluoro-1-methyl-1H-indazol-6-yl)non-8-yn-1-yl)-3-methyl-N-(2-(1-methylpyrrolidin-2-yl)-1H-pyrrolo[3,2-c]pyridin-6-yl)-1H-indazole-6-carboxamide;
N-(3-cyano-2-((R)-1-methylpyrrolidin-2-yl)-1H-pyrrolo[3,2-c]pyridin-6-yl)-1-(9-(2-(2,6-dioxopiperidin-3-yl)-1-oxoisoindolin-4-yl)non-8-yn-1-yl)-3-methyl-1H-indazole-6-carboxamide;
N-(3-chloro-2-((R)-1-methylpyrrolidin-2-yl)-1H-pyrrolo[3,2-c]pyridin-6-yl)-1-(9-(2-(2,6-dioxopiperidin-3-yl)-7-fluoro-1-oxoisoindolin-4-yl)non-8-yn-1-yl)-3-methyl-1H-indazole-6-carboxamide;
1-(1-(3-(2-(2,6-dioxopiperidin-3-yl)-1-oxoisoindolin-4-yl)prop-2-yn-1-yl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-6-fluoro-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-1,3-dioxoisoindolin-5-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(1-((1-(2-(2,6-dioxopiperidin-3-yl)-1,3-dioxoisoindolin-4-yl)piperidin-4-yl)methyl)piperidin-4-yl)-3-methyl-N-(2-((R)-1-methylpyrrolidin-2-yl)-1H-pyrrolo[3,2-c]pyridin-6-yl)-1H-indazole-6-carboxamide;
1-(3-(2-(2,6-dioxopiperidin-3-yl)-1-oxoisoindolin-4-yl)prop-2-yn-1-yl)-3-methyl-N-(2-((R)-1-methylpyrrolidin-2-yl)-1H-pyrrolo[3,2-c]pyridin-6-yl)-1H-indazole-6-carboxamide;
1-(6-(1-(6-(((S)-2,6-dioxopiperidin-3-yl)carbamoyl)pyridin-2-yl)piperidin-4-yl)hexyl)-3-methyl-N-(2-((R)-1-methylpyrrolidin-2-yl)-1H-pyrrolo[3,2-c]pyridin-6-yl)-1H-indazole-6-carboxamide;
1-(4-(5-(2-(2,6-dioxopiperidin-3-yl)-1-oxoisoindolin-4-yl) pent-4-yn-1-yl) cyclohexyl)-3-methyl-N-(2-((R)-1-methylpyrrolidin-2-yl)-1H-pyrrolo[3,2-c]pyridin-6-yl)-1H-indazole-6-carboxamide;
1-(9-(2-((rel-S)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
1-(9-(2-((rel-R)-2,6-dioxopiperidin-3-yl)-1-oxoisoindolin-4-yl)non-8-yn-1-yl)-3-methyl-N-(2-((R)-1-methylpyrrolidin-2-yl)-1H-pyrrolo[3,2-c]pyridin-6-yl)-1H-indazole-6-carboxamide;
N-(3-chloro-2-((R)-1-methylpyrrolidin-2-yl)-1H-pyrrolo[3,2-c]pyridin-6-yl)-1-(9-(2-((rel-S)-2,6-dioxopiperidin-3-yl)-1-oxoisoindolin-4-yl)non-8-yn-1-yl)-3-methyl-1H-indazole-6-carboxamide;
N-(3-chloro-2-((R)-1-methylpyrrolidin-2-yl)-1H-pyrrolo[3,2-c]pyridin-6-yl)-1-(9-(2-((rel-R)-2,6-dioxopiperidin-3-yl)-1-oxoisoindolin-4-yl)non-8-yn-1-yl)-3-methyl-1H-indazole-6-carboxamide;
1-(9-(2-((rel-S)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
1-(9-(2-((rel-R)-2,6-dioxopiperidin-3-yl)-1-oxoisoindolin-4-yl)non-8-yn-1-yl)-5-fluoro-3-methyl-N-(2-((R)-1-methylpyrrolidin-2-yl)-1H-pyrrolo[3,2-c]pyridin-6-yl)-1H-indazole-6-carboxamide;
1-((1-(2-(2,6-dioxopiperidin-3-yl)-1,3-dioxoisoindolin-5-yl)piperidin-4-yl) methyl)-3-methyl-N-(2-((R)-1-methylpyrrolidin-2-yl)-1H-pyrrolo[3,2-c]pyridin-6-yl)-1H-indazole-6-carboxamide;
1-(5-(1-(2-(2,6-dioxopiperidin-3-yl)-1-oxoisoindolin-4-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide;
1-((1-(2-(2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide;
1-((1-(2-((rel-S)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide;
1-((1-(2-((rel-R)-2,6-dioxopiperidin-3-yl)-1-oxoisoindolin-4-yl)piperidin-4-yl)methyl)-3-methyl-N-(2-((R)-1-methylpyrrolidin-2-yl)-1H-pyrrolo[3,2-c]pyridin-6-yl)-1H-indazole-6-carboxamide; and
1-(5-(1-(2-(2,6-dioxopiperidin-3-yl)-1,3-dioxoisoindolin-5-yl)piperidin-4-yl)pentyl)-3-methyl-N-(2-((R)-1-methylpyrrolidin-2-yl)-1H-pyrrolo[3,2-c]pyridin-6-yl)-1H-indazole-6-carboxamide.

9. A compound according to any one of claims 1 to 7, wherein M is of formula (IV): 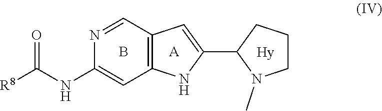
wherein R⁸ is an 8- to 10-membered heteroaryl ring, the group R⁸ being unsubstituted or substituted by one, two or three substituents independently selected from halo, C₁₋₄ alkyl and R¹⁰;
R¹⁰ is a 3-membered cycloalkyl ring;
wherein M is bonded to LINK via a C or N atom within group R⁸ such that a hydrogen atom on the C or N atom within group R⁸ is replaced with a bond to LINK.

10. A compound which is a bicyclic compound of formula (I′) or a pharmaceutically acceptable salt thereof: 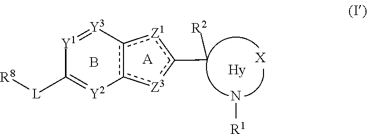
wherein:
one of Z¹ and Z³ is —N(H)— and the other is N or —C(R⁴)—, Y¹ is N, Y² is N or —C(R⁶)—, and Y³ is N or —C(R⁵)—;
Hy is a 4- to 7-membered heterocyclic ring containing X and at least one N atom, wherein: ring Hy is linked to ring A via a C atom within ring Hy, said C atom also being linked to R²; a N atom within ring Hy is substituted by R¹; X is a bond, —N(R¹¹)—, O, S, —S(O)₂—, —S(O)(NR¹¹)—, or —C(R¹¹)₂—; and the rest of ring Hy is unsubstituted or substituted by one or two R³;
L is —C(O)N(H)—, wherein the C atom of L is bonded to R⁸, and the N atom of L is bonded to ring B;
R¹ is H, C₁₋₄ cycloalkyl, or C₁₋₄ alkyl which is itself unsubstituted or substituted with one C₁₋₄ alkoxy or one, two or three halo;
R² is H or methyl;
each R³ is independently selected from C₁₋₄ alkyl, C₁₋₄ alkoxy, phenyl, a 5- to 6-membered heteroaryl ring and halo, or (i) two R³ linked to adjacent C atoms in ring Hy form, together with the C atoms to which they are attached, a C₅₋₆ cycloalkyl ring, or (ii) two R³ linked to the same C atom in ring Hy form, together with the C atom to which they are attached, a C₃₋₆ cycloalkyl ring or a C₃₋₆ heterocycloalkyl ring;
R⁴ and R⁶ are independently selected from H, halo, CN, C₁₋₄ alkoxy, and C₁₋₄ alkyl which is itself unsubstituted or substituted by one, two or three halo;
R⁵ is selected from H, halo, C₁₋₄ alkoxy, C₃₋₅ cycloalkyl and C₁₋₄ alkyl which is itself unsubstituted or substituted by one, two or three halo;
at least one of R⁴, R⁵ and R⁶ is not H;
R⁸ is an 8- to 10-membered heteroaryl ring, the group R⁸ being unsubstituted or substituted by one, two or three substituents independently selected from halo, C₁₋₄ alkoxy, R¹⁰, —(C₁₋₄ alkylene)-R¹⁰, ═O, —CN, and C₁₋₄ alkyl which is itself unsubstituted or substituted by one or two R⁹;
each R⁹ is independently selected from halo and C₁₋₄ alkoxy;
R¹⁰ is a group selected from a 5- to 6-membered heteroaryl ring, a 3- to 6-membered cycloalkyl ring, a 4- to 6-membered heterocyclyl ring, and a phenyl ring, the group R¹⁰ being unsubstituted or substituted by one or two substituents independently selected from C₁₋₄ alkyl, C₁₋₄ alkoxy, and halo;
each R¹¹ is independently selected from H, C₁₋₄ alkyl, and C₁₋₄ cycloalkyl.

11. A pharmaceutical composition which comprises a compound as defined in any one of claims 1 to 10 and a pharmaceutically acceptable carrier or diluent.

12. A compound as defined in any one of claims 1 to 10 for use in the treatment of the human or animal body by therapy.

13. A compound as defined in any one of claims 1 to 10 for use in the treatment of cancer.

14. The compound for use according to claim 13, wherein the cancer is a transcriptionally addicted cancer, such as breast cancer, acute leukemia, chronic leukemia, prostate cancer, bladder cancer, cholangiocarcinoma, colon adenocarcinoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, pheochromocytoma and paraganglioma, rectum adenocarcinoma, stomach adenocarcinoma, uterine corpus endometrial carcinoma, cervical squamous cell carcinoma and endocervical adenocarcinoma, lung squamous cell carcinoma, neuroblastoma, midline glioma or sarcoma, preferably neuroblastoma or midline glioma.

15. The compound for use according to claim 13, wherein the cancer is acute myeloid leukaemia (AML), myelodysplastic syndrome (MDS), myeloproliferative neoplasm (MPN) or acute lymphoblastic leukaemia (ALL), for instance wherein the cancer is mixed-lineage leukaemia (MLL) rearranged (also known as KMT2Ar) acute leukemia (AML and ALL), NPM1 mutant acute leukemia, RUNX1-fusion acute leukemia, E2A-fusion acute leukemia, PML-fusion acute leukemia or NUP98-fusion acute leukemia.
